SEQUENCE LISTING (111 sequences)

SEQ ID NO: 1 - protein, 225 aa, Homo sapiens
MDWGTLQTILGGVNKHSTSIGKIWLTVLFIFRIMILVVAAKEVWGDEQADFVCNTLQPGCKNVCYDHYFPISHIRLWALQLIFVSTPALLVAMHVAYRRHEKRKFIKGEIKSEFKDIEEIKTQKVRIEGSLWWTYTSSIFFRVIFEAAFMYVFYVMYDGFSMQRLVKCNAWPCPNTVDCFVSRPTEKTVFTVFMIAVSGICILLNVTELCYLLIRYCSGKSKKPV

SEQ ID NO: 2 - DNA, 678 bp, Homo sapiens
atggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtt

SEQ ID NO: 3 - protein, 226 aa, Mus musculus
MDWGTLQSILGGVNKHSTSIGKIWLTVLFIFRIMILVVAAKEVWGDEQADFVCNTLQPGCKNVCYDHHFPISHIRLWALQLIMVSTPALLVAMHVAYRRHEKKRKFMKGEIKNEFKDIEEIKTQKVRIEGSLWWTYTTSIFFRVIFEAVFMYVFYIMYNGFFMQRLVKCNAWPCPNTVDCFISRPTEKTVFTVFMISVSGICILLNITELCYLFVRYCSGKSKRPV

SEQ ID NO: 4 - DNA, 678 bp, Mus musculus
atggattggggcacactccagagcatcctcgggggtgtcaacaaacactccaccagcattggaaagatctggctcacggtcctcttcatcttccgcatcatgatcctcgtggtggctgcaaaggaggtgtggggagatgagcaagccgattttgtctgcaacacgctccagcctggctgcaagaatgtatgctacgaccaccacttccccatctctcacatccggctctgggctctgcagctgatcatggtgtccacgccagccctcctggtagctatgcatgtggcctaccggagacatgaaaagaaacggaagttcatgaagggagagataaagaacgagtttaaggacatcgaagagatcaaaacccagaaggtccgtatcgaagggtccctgtggtggacctacaccaccagcatcttcttccgggtcatctttgaagccgtcttcatgtacgtcttttacatcatgtacaatggcttcttcatgcaacgtctggtgaaatgcaacgcttggccctgccccaatacagtggactgcttcatttccaggcccacagaaaagactgtcttcaccgtgtttatgatttctgtgtctggaatttgcattctgctaaatatcacagagctgtgctatttgttcgttaggtattgctcaggaaagtccaaaagaccagtc

SEQ ID NO: 5 - DNA, 500 bp, Homo sapiens
acctgtctcccgccgtggcgccttttaaccgcaccccacaccccgcctcttccctcggagactgggaaagttacggagggggcggcgccgcgggcggagcgcgcccggcctctgggtcctcagagcttcccgggtccgcgaacccccgaccgcccccgaaagccccgaaccccccaagtccccttcgaggtcccgatctcctagttcctttgagcccccatgagttccccaagtgcccccagcgccctgagtctcccccggttaccccgagcgccgcctcccccagccccttggcggcccgggtgaagcgggggcggctgagagtcgggaccccccaggaagcggcgccccagaccccggctccggcgctgtgccgtgggcggggttcagggatggctgtggtcgttgtcctctgtactccgcatagtgcgagaggacttggcatttatgagcgcttctttaattttttattgttagagaaacaggcattcctccaagga

SEQ ID NO: 6 - DNA, 4843 bp, Homo sapiens
ctttgtggatggcttggtggcctcactgtcaggctggcactgatggctcagttagcatatctgttttgataagtgctgcaacagtgcattataattgtgggctgtggttttaatttcaaagtgtttcttaaaagacacattattttaaaatgacagaaaattcaactccctcggttactggcccagctaagcgacgtcactgcattgcagttcagcgctgaagcttgggagagtcccacactccttactgcaagcggatgtggagaggccagtggataatctcctgtgagcccatggccttcttttcatcccaggatgtgaattgtcttcactgattcatagttacaccctgcctgccacaaccaacgctctcctaaacaagattccaccctctccacaatccggatgaatcatctcttttccacccttcagagctggtagtgaatcctccttcttctttttcttaaaagcatcctcctctcctcattttaggcaagttgcatcccgttttctgatggactccagaagcaggctcgtagtgaatgtctttcatgacccacagtcgctgccacggggcaccaaggtcaggcagaaaccatccagtgccaccttggtcagaggctaacaggagagaggtggccacgaaagttacatcagattgacataggcctgtgaaacatttagcttcactgagcttgggaaagacaacatcattggaaaaaacaatattttagcccaggttcagcactgacccattgataatccagactgggaggcccttaggtgagctggttgtcctgctacagcacccacagctcaggccagtcccgtcccaacagcagaaccaccgaggacagcaacattccgattttaacaaaagcatcttatggaattagacattcttcattggccctcactgagtggaaaacaggatactccccgaagtaaactctctcctggtttacaacaatacacctggccaagaatatggggctgcaggaggaggggtttatcctttgccctcttccacctgccaaacccaggtcatacacccttctacagacctgtccagttaccatcagctgagaaaaatacagttccgagaaaccctatattgttattttataaagcttgagttgaagctacctgttttaaagatcctttttcaggaagaggagtaaattaagatttactccccaatgggctagggggtcatgggttaagaggggctcagaagcaggacgaagttgttttcaatattcaagtcagaggaggagctgccctcctggcctcccgaccctgggcggttacatgcagcttcctaccgggcccacgccatcctgcaccgcctggagggctgccagaggccagcggaggagttggttcagttccttagggaagacactaggtgaatcaccaggatccagaaaaggcaaaagggactcttcaccccttaaatttctccacccttaggtgatgggtggtcgaccttgcctggctgtccccagagggttcctccacccttctcaccagtgtctgaaattgtgaccgactgtgcacagcagtttcgaaagggactctaaggtcacatggggacacggccgtaccacgcttctcaaggcagtcccaggtgcatggccacggaacccagctctcagcagctgttagttaggtgagcgctgttcgggctgccttcctcctccagtggggcaggatcgaggcactgatggaaccgtcctgaggacgcgggtctcagccgcacaccacctcttcgcgaacaagggtcctaaaaattttccttctaggcggggagcacagcccggaaacagaccctcgtgaagtgtttaggaaaaagggaagccactgaaatcttggccccggggtaggccgggatcggctggctccgcgttagttctaggcaaactccgcccaaatctctgcccggggatttttctgcagaagccgctccaagaggtaaaggtcagttcctgcagcgaaggcttcctgcttcaccggcgaaacggagctttgcttcgaagctaagctttcggtgaatttaaaacgtttggtggcagtgggtcaagtagccaggcggctgcgctagagtaccccgaagggacatcggcgacaccacaaacctcgcgctggcggctcgcccgcgcctttttcccctcccgcgcgcgcccggccccactcgcaccccgggcggtgccatcgcgtccacttccccggccgccccattccagctccggagctcggccgcagaaacgcccgctccagaaggcggcccccgccccccggcccaaggacgtgtgttggtccagccccccggttccccgagacccacgcggccgggcaaccgctctgggtctcgcggtccctccccgcgccaggttcctggccgggcagtccggggccggcgggctcacctgcgtcgggaggaagcgcggcggggccggggcgggggtctcggcgttggggtctctgcgctggggctcctgcgctcctaggcgggtcctgggccgggcgccgccgaggggctccgagtcggggagaggagcgcgcgggcgctgcggggccgcaacacctgtctcccgccgtggcgccttttaaccgcaccccacaccccgcctcttccctcggagactgggaaagttacggagggggcggcgccgcgggcggagcgcgcccggcctctgggtcctcagagcttcccgggtccgcgaacccccgaccgcccccgaaagccccgaaccccccaagtccccttcgaggtcccgatctcctagttcctttgagcccccatgagttccccaagtgcccccagcgccctgagtctcccccggttaccccgagcgccgcctcccccagccccttggcggcccgggtgaagcgggggcggctgagagtcgggaccccccaggaagcggcgccccagaccccggctccggcgctgtgccgtgggcggggttcagggatggctgtggtcgttgtcctctgtactccgcatagtgcgagaggacttggcatttatgagcgcttctttaattttttattgttagagaaacaggcattcctccaaggactgaagatctgttcgagtcgcggaggctgcgcgggcccgcgaggctctcgcagggggacctaggctgggtggcggggcagtgccctctggaatgggggttaacggtggccgaggagggggcgccgctggtgccggcgaagtccccgcttctttctcccctcaaaatctcaccaatccgaacgaacgccttctcgaatttccgattttattcaattactttcaacaatgtgccaaggactaaggttgggggcggtgggagagacaagcctcgtttttgccatggccggcaggggggtcccgccatctgcggagggtgccccccgcggcccccggcccagccaacttcctcctcttttcgcaactggggaactgcaaggaggtgactcctttcggggtgaggaggcccagacttttcagaaaggaaagagggcaggtaaaacctgccaagccccttcctgctcgatgcacacagcacgaaagggggaaactgataggattctgcggaagaccgctggggggctggctctgcactgcacacctgctgggggctttctggataccgtgaaactttgtctcagattatgaggtctcagtatttgcatttggttggggattttgatgtcttgcgatacaaatgacagaagacagatttgcacagcgcaagcggatgagggactaagatgtgcagagcaggctgggtggggactcccggggaggtctcccccaacccccgccccacctcgggcacccacttcgcgatttttgcagaggggagccaggtcagaggtgcagcctggtcccctcgcgctcacgtttttacccaggtcagttcgaagttaagtggaaatgatgattaatcctgacaagtcagatctggcctcagaatggatttcccgtgattgccaccattattagcattgacttttccttgaaaaattggcgccccgtggccatgggccgacctaggcagtttctgcagggacgagcgtgagttttgtaccgcggttaccacctactttccagctccaggtcttagtctaagagggagtgtctgctcatgaagaggcaaagccccaggagctgcgaaaagccttgcatggcccatctgagagatgtgctgagtcggcttgttaaaaatgacaggcaaagcctgtggggtggggcagctttcttggcctgagcgcatcttggttgagccagaggtgacttggggtggggagtggggcgccggttggtgggttctccctttaatttctcaaaggctgtggtgtttatgagtctgttggaatcctggttgggttggaatgaaggaaggttctagaaccattgtgggaagctcgctagtaaagatggtttggagatcggaagttgactgactttcccccattgaaaaatgtcacctgagattttagtgcctgtatcacgattataggctcaactttcttttccttgttttctttgatttagttctccttatgtgcaaaattactgtgtgatgttggctagtcgtattatcacagccactccgtgttttcaggatttgtagctggaagtcctatagcacttaagtcttcacttacagatcagcgcttgcttttattctgttttgtgtgatttctgctgttttcctgtgagttggtgttttcttcccaagtaggctcaggactcctctagggcaggacattatatgcatgtacatagtgtcctccagtgtaggggaggagaaggaggagaggtgaggtgggaaaagggtgaggg

SEQ ID NO: 7 - DNA, 5178 bp, Mus musculus
ccaaaaagggacaaaaacagacaaacaaacaacaccaacacaaacaacaacagcactaaaacgagtctctgcacctaggtcttcgcacgcaggctggtagtcccaccctcaggtagggcctgtttggttaacgatccgtgtctgttttgatatgtgttgcaagtgagtgttgcactgtggactatggttttaaccttgaagtgattctaaaataaatatatgatgaaaaatgacggaaaattagctcagcggttcaccagttgctggtccaaggagccacctgatgggggttttgccttgggtggcatcacagtgtatcctgtctgagtgacacagtgtctatatatggcctgtgccctagatgagcctccataagccaatgaccttctatttcatcccagggcaggaaccttccatggctacacctggtctgtcacaatcaacccctcttttgattaatcccatcttcccggctgtcctgactcacttgcttccaccccttccttccaagctgtaaagaatcctctgactctttcttaaaagcaccctaccctcctgcttagcaagttacatcctgtttcgcagtggactcacagcaggcgcagagagaagtccctccttgtccctagtggcggtggcagagcaccagggaacccacttgctggaacccactcagctctgccttggacagaggagatagggccaggggcatgggaattaaggaatactgacatacaccggtaaaacatcaagtcctatccaacttggaaagcagaaacagacaggctcggcaggttcagccctgacccatttatacctagactgtcagaggccctttgggaagctggttgtcctctgaacagtctctcagctccatgtggtctgcccccaacagcagaaggattgaaaagcaacagtgttccaagtttaacaaaacaatctgattggaattagaccttctgttcttccttccccttctcccgagtggagatcaggacattgaaataaacatctacacacctgacccaaaatacagagctggaggatccctttgcctgcctatagcatccacagactagcccaattattatcaacacagaaaaaaaaaaaaaccctcaatttctgcgtaaactgtgcacttgtttataaaagtacttaagtgtttgttgaatttgagtttaccgtgttacccaggatggcttctaaatccatgcagttggagttagcacaacatgggggtgggggtagggggttaatacatctataatagcagaactctggaggctgaggtaggaggagtgtgctaacttgaggaaaacttttctgcagagcaagaccctggctcaagaaaacaaacaccaaaagagacaagaaaagaaaagaacagaaccaaaacaaaaacaaacaaacaaacaaacaaaaaaccaaaaaatgggaaggccggattgaacaaacaaggtcaagaagagagagagagagagagagagagagagagagagagagagagagagaaaactccaaaagaaaaccaaatagctgggacatagctgtgggtcccggcatatctgattgcagctgcttgtcttaaatggcctttctaagtggaaggagaggttaaaatttgacctcacaaaggggttaggagtactaagccagcaggtgaaatcgtcaatattcaactgtggtgtaggaggtgatttccaggctggccttaggactaggtcacacgcaggtccctacctggcatgggacacctggagattgccttgaaccggtgaatcattcgctcctgagtagaagggagcttctccatgtttatagtatatactgcatatgacccttatttgccttaaaggatacttcggggagctggtggactgcctctagatgctgaccccaccgcaccctccacccttctcataattcactggctttgcccatagttcccaaaggactccggggtaagtgtagccatgactgagccaggcttctcaggacaatcccgtggacctgagcaatgggtcccatttaggcctacgctcccttcccttccattgaggcagcaccaaggggctgatgcaattgtcctaagggacaagtttctcagcagcacgccatctgtgaacctgtgccttcccttccagctgtaacgtcccgcctggacgcaaatccttaaaaagcatttaaggaaagaaaaaaaaaaaaagcaatcaaaatctccacccgagtgcaggttggggttccccagctcgcgggagcggctacggccgcgcgttttgggcggtcgcccacgtcaccccagtgctttaggtggtaaaggtcagtgtcttcccacggaggcttcctgcttaacaaatgaaactgagttttcctgctcagctttcggttagctaaaaacttttcaatggcggcagacaacgcagccaggaggcctcgggaaaattctagcgaaggaatactggcgacacgtcgcagtcgtgcgcggaacagcctggcccccgcgtccctccccaccccgcgctgtgcgggacctcccggctcaggctgtgcgcggcggtgagagcagccggctccaaccccgagccgggccagacgcctgcagccgaagaaacgcgttcacagctcgggtccctatgcacgggtggcggtggcccgtagggaccgcgcagcgcgttccggcctcggtttcccaggaccgtggcggcccgcacccctcctcgcacctcacgcgtccctactggctgagtctcgcgccccagccaccgtggggcgttgcggtcgggggcgggttacaccagtgtgactcggtggcgcggattggcggtcgcacctgtgtccggaggagcgtgcagcgttgggtggcgggaagcggcgaggcgctgtccccggtaaggagcaggtctgaagcgggtcccggggccgctcctgggttggtccgaaatgggtcgccggctgatcctgtgctggtcgccgcgggtcccggtggaggctgcgctcagtggactggagcgccgccgactggctgcgagttgggagagcggagcgcgccgcgcgctgcgatcctggacacctgttggccgcggcgccttttaaccaaagccctcaccccgcctctctcaccctggagcgattgagaaagttgcggaggaggcggctcccagtagcccgccacccccagcgccacgggcggggctctccgggcacccagagccgtcagggcccgccgagtcgcgagctctcctggagcctaggtcactccccaccccactccgccccaccccacccccagctctctttgagctcaaggctcttccagtgtcctgtcccgagcgcagcctgaacagagctggtagacctgtgtcttcacccaggacgcaggtcgcaaagctccaagtcccagctactcgcttttgggggattgggtgatgttgaaagagagttgatgttgctcttactactctcactagtggaaagtgtgctgttatattcgaagcttcgctgtagtaatattatatatacttgtgtgtgtgtgtgtgtgtgtgtgtgtgtgtgtgtgtgcgtgttagataaacggacggtacagttttgtgttggcctgcagcttccagtagcgcacaggagactcctctcccgtagtgcagtgagctgaggcatctagaattcgggttcaaggcagactaacagagggcgccgccagggctggccaaattctggcttctatttctttgaattcccgatttaattcgatcactttgaacagggtgccagtggctaggacagaagaagatgtagaggtgcgtctccagggctggcctggaagtggacttgtcacagtctctggagggttctctgcctgtgcccccgctctctgtgtcctcttttccacaactgaaagcattgcaaggaaggggcacccagattctgccggtgcaggggatgcggaaggggggggggagcagaagaggttaggcaagcccatccctcttggagtccaggatgctgggaagacctgggcagcctgcatctacctctctccgccaagctgttcgtgggttttgagggctcggtgttccacattgcttggctgtctggatagttttgagaggagttacggtggacattcacaagagctagctacgctttgggatacctaggccagctagcttcaccttactacttgcaacccgagtcctacagctgccaggtttggaatgaaaacggcacatccccacaaagttccttcagattagctttacacgcagtgaagagactgattcattctgacaaggcccgtctggtcgaaggattggctttcaatgaaaggaccatggctgaaggtacatgctttccctgtaaagctggcacattgccgcgggcagacctgactgctcttgcttgggcagaggaaggttgcacgctcgcttgctactacccccacctcctttctaactgtaagtcttagtctaagagggagtgtctctaaggaagagagcctcggatctgtgtccagcccttcagagagagagagatgtgctgaatcagcttgtgtggaataactggccaagcaagatggggtggtacaactcccttggcctgagcacatctaaagatgaatcaaagaggagatgaggtagtggcagcaggcaggggtggaaggatgttggcacctttagcttctcatgggtcgtacagtttccagtcaattggagcccctgttcagtgaggatgacagaagcttctagaatcattgtaggaagctggccagtaaaagataggttggagatcagaactgcttcactttctccattgaacaatttctcctgagggttagtgcccacgttatgattacagcttcagcgtctagctccctaacttgcttctacagattcgcctaatggctgtgtgttggctgatggtcacaggtgctgggaatattaggatgtatcgctagctcatctcctcctctgttccagccatccctccttgtttcttgttttctcaccaactagaccagaggctcctctagggtaagaaatgctaaatttatttgtgtatgtgtattctccagagggggagaggggagagggaaggagaagggaggggaagagaggcaaggagaagggagaagggaggagaagggaggacagggggacagaggaagctagaaaagagctagga

SEQ ID NO: 8 - DNA, 4964 bp, Homo sapiens
taatccagatgttaacactgaaacttccaagcaggggagtgaaatgagactttcacttttgacttcgtatactcctgtattatttaagtgaaaatgtatttatatattctataattacaaaaatcacattggttgccttttcattttgaaatgagcaaaagtgacagggctgttaaaaagctaagtcacttgagcaataacgtgatgtccagaacagtggttccatggctcagccatgtcgggggctgcactgaggacagggggccatctgccttctaggaggacactgtggactggaatattgttcctgccttgaggaggagtctcccagcacagttactgctgcttgactgtcagagcatgcgttttcttagggaagttgaaggcagcctgtatctagtaaggtggtatgcagtagttgcttaatgctgaatgtgtgaaggaatgtggggctgtggagcaggaggataaagtctgaacttggacctgttgttctcagctattcgaagctttctcaagtggaaaatagactgactttgggtccatcagagggcagaacaaatgctggagagcagatgctagaattccgtcttaaaaccatgaatccttacagcggcctgcgtggcctgcgccatctgtcccagccacgccctccttggccccatctccccctttctcgccctgactctttggcatcctggcctttccgtctcactgggatgcttccctaagagactcgtgtggtttgctgccctgtatcctccggatctcctgaccaccctatgttagttacattgcaatttcccgtttccctcatgacgtcttattttcctccatttaaattacctgcagcaggtaccacctacagggatctgttgagagtcggcctccttcaatgtgaagcctgatgttttgttctgttcacagctatgcccccagcccctaacagttggtggcagtcagtaaatattgcctgggaaaacgaatcattagccatgtgcagaaatggaacagcgtctcaccaagttggggttgcccctggaccctgtgaacactggggcagctggggtgttcctactgtgcttgttaccggcttcaggaatcaaatgcactagagaattgtagaagtgcggtccacatcctctgtgtggtaggaccagctgctgttggcctctgagcaggatctcttacctctctgagcagtgccttcctgttgccctcagcaagaataacactaacagcctaggacttcagagcactgctgcgaggtgcaaatgaggtgatatgggaaaagcatttggtgagatgtatggaaagtgtagagaccctgaccagatgagtcaatggccttcttcgttactctgttgacctttctttaattacagagtcgcatagctgtcaccaccttatccttttttgctgctatatttgcccccagccattcctctcccggcttatgtggctagactcacctgcctgtgctgcagttactccaggctttgtgtaaatgtgcatttttttccagcccccagtttatcaagctttgcttgagtcacttgtatctgaaataccatctgtcactcttccaggttgggatctgtctagtggaaaacagatgacagtcatatgttacttagtgctttactatgtggagaacgtttacataaattatcttatttcattgccactaagccggggaaagattcaggaaacccattttaagatgaggacactgaggtcagggtaagtgagtgagcttttacccacctctcagctgctctctagttgtcaaagaccaacccgtgggggtggctcaggcccgacccctgcagcatattccttggggcctcccaagtgggcccgatctgctcaccccagctgtgactgtcttttgacaggaggagggagcagcgaggctgcacccactgctcataaaaagcagagcttgtccacgccgagggctcggctgggtgggaggccgcttccacaaggctttttcttgctccatacaaagtgcagactgatgctttgagatatagtcaggattatcattttcagagctcaagctctaatttccaggcatgtgaccagacctctctatccattcctacaagtggtcgagagtagcccataattattttggcttggtcttttaatagcttgagagtaataatctacatagcttgtagaagtgaatgtacttattttaaaagttctgtgttttttgatgttgttgttgtttgggacaggatcttgctgtcgcctaggctggagtgcagtggcacaatctcagctcactgcagcatggacctcccaggttcaagcaatcttcccacctcagcctcctgagtagctgagactacaggcacatgttaccacgcctgcctggctaacatttttattttttatagaaacaatgtctccctatattgcccaggctggttttgaactcctgggctcaagtgatcctctcgtctcagcctcccaaagtgttgggattataggtataagcctctgcacccagcttaaaaaatcctattttcacagtctatgtgcagagcattttggaagtcaggtagaaaccatttcccattttctattacctgggtgatagttgactggtttttgttctttgaaatccattttaaaagtgtatggtcctctatgaaaatacttctaattattgatgtgtgaaatgctttgaaatccttggatggaaatcttgtaccatgaaagaacagaactgttggtggtgtctctgggagaggctcacgagggccgggcaagcctgtgggggtagcaggcagtcactcccatggggacaggctgacctggcaggcttatttcccatggaagtgggcactgaggaataaaaagcagtttcaggccaggtgcggtggcccatgcctgtaatccttgcactttaggagactgaggcagggggatcccttcagcccaggagttcgagaccagactgggcaatatagtgggacctcgtttctacaaaaaatgaaaaaattagtggagtgtggtggcacactccagtggtcccagctacttgggacgctgaggtgggaggatcgcttgagcctgggaggcagaggttgcagtgagccaaggtcatgctatgagtaacattttgaaggtccacttctgggattcatccaggagctaaacgggtcatgtccagccaactcagcattcaccaaggtacgtttccagaccaaacaccacattgtccatagactgatatgcctcaaaaacctggtagaggtgggcacggggttaggtagaaatcatcttcctcccttccttccccaccaaactttctggtgacagaagcttttctgtaactggggcagaatggggtcagacactctggcaacttacccattggtgttatgaaatataaaacattaatgtatttatataaaaagtgatagatgaaattaaaatttgctgttctattaaaaccatattagattttaaattattatagagattatattttaatgttttaaatgtatttgatacattacaaaattattttagttacaagcatatcattaaagctattctttattattacaaaatgcttttacaatgctattcttgacaacaggaaaatacttaccctcactgaaatatgtggagtaccattttttggaaaccatgtcaagcataatggcaatattcaggttcaatcttcctatagatctgctcaatatttatctaaaccttagcttctattcttttcacatgttattagctatattttcacttaaaaaattggaggctgaaggggtaagcaaacaaacttttgaagtagacaaagctcatctttaatcaacagactttagagtccagtctttccaaatctgtttttaacgacagaaacttctccctcccctgccccattttgtcctccccattaaatggtactgtgtcaataaaattcccaagcgacctctttaaatcagcgttctttccgatgctggctaccacagtcatggaaaagggagatgtgttggacaggcctgtcattacaggtagtagttggtggtacatccagtctgtatttcttacacaaaattacatctaaatatttgacatgaggccatttgctatcataagccatcactaggaacttctagtctgtctcactcgattgaggctacaatgttgttaggtgctatgaccacaatgaatacaacagacagcctctcagctgtgctgcaaagtattcataaccaaaagaccatatttcaaattaaatcatagtagcgaatgacataccatttacatattacaatctgagcctctgaaacagggggaacatataatggtatccagaacatctttacatcaaaataacctatcatactacaaagttttcacttccaaaaagtgtaacagagtttaaggcactggtaactttgtccactgttagagattaaaacttccaaagcaaatgaaagaaccaatgttcacctttaacgtggggaaagttggcaaaaagaaccccaggaggacacccaaaccttctctgtgtcctctgtggaacctggcttttttctcttgtcctcagagaaagaaacaaatgccgatatcctctgtttaaaatatgaaagtaccttacaccaataacccctaacagcctggggtctcagtggaactaacttaagtgaaagaaaattaagacaggcatagaattaggcctttgttttgaggctttaggggagcagagctccattgtggcatctggagtttcacctgaggcctacaggggtttcaaatggttgcatttaaggtcagaatctttgtgttgggaaatgctagcgactgagccttgacagctgagcacgggttgcctcatccctctcatgctgtctatttcttaatctaacaactgggcaatgcgttaaactggcttttttgacttcccagaacaatatctaattagc

SEQ ID NO: 9 - DNA, 5166 bp, Mus musculus
catggagagagatggataactgagatttctgggcaagagatgaaatgggctgaatcccactcctgactgcacacacctctcagtgatttaattagaaataaaaacaagtctctacattaacatttacataagtaacatcagccgtcttttccattcaaagtgactgaaggagatggtgttgttaaaagattgaaattagacagcagcaacacgtctagaagagcatccctggggcagggttctgcctcaacaccacacagcactacacagcaccacacttagcacaaggctcctcgtggctcctcatgtcccttcagcaagtcaccagtgcaccaggaggcgttggggagggaactcctgaccacaatcacagcctgagggttggagttgtgtttcagtcatcctggggggcagggggagcttaaactcgttggcatttactagggcagtacacagcagccgctccacgttgaacgagtggatgatcagcctgagaatcaaggctgggctgagcttggctctatcctcaattatctgcagagcgccctggtagagaacagatctgcctttgagtttccaagtgagagcggagcaaggctgggcacagagcagggtggcaaggtggctgctgtgggcacagcacagaagatactcaggggcatagatcttcctggtggctgcttggtctcatgttggtcaggtcacctccatttttggcctcatcatcttctgacatgcacctgcttcatgcgtctgcttcctggaacccattcctggctttttgtcttaattctctgaggcaggtggctccattgcttgtctcctttaggtttcatctaagagggaccgtcacacacagcctgtgtgggcatcatgctggtgcctgacagtcctctctctctctctctctctctctctctctctctctctctcccccccccctctgctgtggctttggcctctgcagaaacaatctatgggatttgttgatatgctgcctccttcaacacaaaggcttaagttgtatttatcagctccagtcccagggaataatcatgtctggtgcttagctggtgctcagtagatagcagctgatgaaaaaaaatcaggagggatacgtaggaactgaccacaaaatcttgtgggggtgcagttacaccacggactccagcagtgttgcaacagatgtaggttgtgggcctgtggagttagtcttcattgtgggaggggcaactccacaaggcctatcaacataacctccgaggggttggactactcttgctggccttcgatcttgacaattaccagtgccttcttcacaacccctcccccacccctgcacaggtgatgacttgatggttcttaagttgcaataagaatgacaggaagcaagcaggaagcaagagatgtgatatacacattaggtcgtatggagaccctgacagagcaaacctgtaacattcattcttactgtattagcccctttcttagtcacttattaatattcatttagtcatttagtttttgctgtttgcttgatgcagagtctcatgaagttcaggctggctttgaactaagtatgcagctgaggatagccttgaacttcaaattctcctaccttcatttctgagccattgggaatgcaggcatccaccttggagcgccatttctatttatttactttctctaaggctggggatggagcctatggctgtgtgtggtaggcacaggctggggatggagcctatggctgtgtgtggtaggtagcattttggcattgactcacttactctccagcccttgattcttttgagttacagagtgataccattgcctgtcactcatctttactgtgcttttgtgtatgcacccagccccccttcctctgttgacctggctggtctctgaggtcactgtgttatgtttatttcagtgtcaacctgcacactctcaagcttccggttaattgagctttgcaggagacattcctacttactctgtcattcaccatgtcactcagggtctactgagtgggagagagatgacatattaatgctaatatcattctactgccctaggtggaggagagggtctgtgtgaatcaccccattgcttttcctaggggtggggagtatttaggaagcccactgtaaggtggagagcctaggccagggtaagcacggagctcccttccacccgtggccacccattcagcatttgcaagctgctccctggtgcatcacctagttagaacagtggcacctgagacagcttaggcctggggaaaccaatagaacactctgttgttccacttggactagcagtggcctgtctctccacagggagcaccacccatgttggggagcatcacctgtaacctccagagttcactcacaccaaggcttcttctcttcacaaactgccatctgctagtatcaggatgatcatattccagaggccaagcttatggccagccctctccgtcagtcctatgaagtggttgttggcagtttgtaattattttggccctgttctttaataccttaagagtaataatcttcataatgtgtaggagtggaactagccatttaaaaagctgtgcattcttttaacagggtacgtccaggacaccctggcaggtgggagagactattcactttttctactgtccaagtggacgtgggctaagttgtatccctttcgagctaggttgtatggtcctccataaaaacatagtatcactgatgtttaaaatgccttgacagcctcagtgtgaagcttataatttaaaggatgatagtgtaggtaccacccaggagagagacgtatagcctgtcccttacctgggacacgcttgcctggcaaggtctgtcccgtgggaatagacatggaggaaacaaagaacatgggccacatgcttctacacacacacacacacacacacacacacacacagagagagagagagagagagagagagagagagagagagagagagagagagagagagagtcttgcaaagttctgcagaggacggttctcaaagtgtagtcttcacagtggaagatgttttaatttttaaatataaagaggtttgttgttgttgttttctgtgatactggtgttccaatatgggggcccacacacggagacaggtgttttagcgctgattacacactgagcctaaggaccatgtaaactgtgagttcctctgcttcttctagaaacggaacggaactgatcccgtcaccaggacttagcatcctcctgctgcactctgactctcagaccttgcagcccttaggttggggctcacggaacctcttagagtgcgtggatttgggcagcagtggtctgtctgttccctctctctttatcaagttttctagccacagggtattttttgtaactggagcagaatcccagaacatgttgtaacatgtgagcatacttctgggatgctttaagatataaactatgaaatatatgtatatacaaattagtatagctgggcatggtggtgtgcacgtttaatctcagtccttgggaggcagagacaggcagatttatgagagttctaggccagtctggtgacagagtgaggccctgtttcaaagacaaaaacaaatcaaagccagaaaaacttaccattggtcacgttagagtttggtattctattaaaaaccttatttaattttaaagtatacaaaataatcatattttaataaagggcatttaggggtttacaaaattatatcagtgacaagcatgaaaccacaactcttatttattgttacaaaatggctttccaatgacattcttggcaggaagaagtgtcccctgttggatttgttgactgtcatcttgtaggatacacataaggcatagtggtaatggttcaacttgccctagaaaggttacatactgacctaaactagtttcttctatttcttccaaatatccacatttctgtttccagttaagaaggcaatgctgaagagggaggcaaacacactttcaaaagtagaaaaacttagttttaatcaacaggattgggagtctagaagtttcattggttctctgaaaaccaccccatttggtttctgcaccattgaattgtcccatggcagtgaaattcccaagcaaacccatgaagtccctatcttctgatgctgactgcaacatcccacagctacagagtagacaaactggtggggggtgggggtggggtggggctgagttaggctcatggcaggtggcagttgtcggcatatcctatctgtctcttacacaaaattacagttgactattttaattgaggcctcttcttgtcagaagccagcacgagacgcttccagtttgtctcacttatgacaggcagtagggttatagccctgagcccagcacgccagtgatgaatacaataggtgggccctcagccacactgcaggtttcccataacccaaaggccaacatcttaaagaccctgtgagatctggttacacaccatgctcacttcacacactgaacctctggactaggaggaatgtataatactttccagatcattttaggaaaaaaaagagcctatcttattttaaggttttcattaaaaaaaaaaagtacacagcacttgaagtattaatagctttttgtccattgttgcacacgtaaactatcaaagcaaataacagtatggcatttctttacctttagctaggggtaacttggggggggggactttctcagtggcaccttcctcaggaccgggttcctctctcctgtcctcagaggaagagaaacaatgtgagatccctttgtttaaactgtgaatgtatcctccaagcttggtcgctaccagcacggggtctcagtggaactaactttagaacccattaatacaggcatagaattgggcctttgtttgggagctttgggggaagggaggcccacggaggcttctggagtttcataggaggcctccagggacttcaaatggtggcattttagatgggaatgtttgtcttgggaactgctggtggctgagctctgccgactaagcgactaagcatgggttgcctcatcctctccctccatctttgctctagcagccaggcaatgcattagactggtcttttggactttcctgagcaatacctaacgaac

SEQ ID NO: 10 - DNA, 2504 bp, Homo sapiens
aaggggacaggacatctctttccaaaacttaggtttggtgactcctggatttcacactctctgactgcttgggtgagggtggaatggagggctgtcccccaccctcgcacctgcacggtggcatgctttcctcctactccagggaattcctcgtggcctcatggcctgggctgtttctggcttcaagctccacgtggcctggccccagcggtctggtccaccttgtactcggtgcccccgctgccccctggcctcagctggagtgacgcacctcatccatgcgggcctggcgtctggaaggtggctgggtctctcgggcttgagcaccatcatcttagctccaacatgtcattattccttcctcactgaggacttttctgcttcctaattggttgttgaagatgaggcccccatgctcttttaagaaaacctgttgtgccccaggcttggctgtgatgggcactgactcatacagaagtagaaaggcctgctgagtcatcaacactcgtgcgacgccctcgcattttcattaatgatggcctccctgccacacgtgaatcactccagcccgagatctgaaaccaggacacaccccaggggcgaggtgacgctgagtgagcccagctgtgtccctttcatgagaactcagagcacagggctctgtgtgcatggccgtcccctccagagaggaggaagtaaatgccgggattagtggaagatcatttccttctatttgccttggcttacgtctttcagaattcaaacacgtgcactgttgaccctgcaatggtggagtttttggattttccttcagtccgattgctaaaatacttccctctcatgtgagctgttgtgaaagtcatcagccagataccattctaaaaacaaagaatgtgcttctcgtatgttgcatgctggttactgaaatattagggaattacataaaggttttctggggcacatattcaagctgaatgataaaattgaaggtcacacaaagctaaggtctttcaaatcctgacccaattagctctctgttagctctctgactttggacaagctgtctggtcctctgaagcatactttgttcgccctgggtaggggccctctgttttaacagcgtttggcagatgaaaacatttgcaaagccaaaggacaatgaaatctacggaagcctaccatatgccaatgactccaccaaatgttttctcttcttgggatcttctaaaattcatctgaatacttataagttatgcaaattttggttattaatctaggttgtattaccttgggggaagtcagttaatctctttgaactcagtttctttatctgtgaacctgaaagaacaccttcaaactccaagggtggctgtcagaattaactatagaggtgcaggtatcagatgaaagctataaaacagtttacagatcttagatattatgatggatggctatgatacgtttctcgaatcactgcttgccaatgagctgtacaatcttcctgaaggggtctgcctttccaatctgggcagcaacagttaatgacggtgtgccaggatatctgtgtctccttttatctgctccagactttaaacacaccctctgattacatcacactatcaatttgaaaaagggctcagagccaaaatcaccactgttagcgagttctccagggctgcctcctatcctctggaggtggggctctcgtctgcagaaataggcataagggttttctatggtttttgtttgttttaaagacgaaacatgttttgggatcttttaagaatcctaatcgttgtgaaagaaactgaagtaagttactgttcaagtgactctcattctgctgtgaatagtttctcccacgtgaagtcagctcaagagactgtgaattgcttcagcctacctgagacctggtacacagggaggcttcctagccacggaagaggagagcgtttgcaggaggagaaggaggagagagggcccacgcaggtgacattctggaaagggaatgctggtgcgaaactgcctcacctactttgctccttggatgttcaggaaaagccagccccatccgccccagtccgagggcctcactcatggaacaaatgaagctgagaagaggagcttcctgttttccagctgctggggtcatcattatcttcaggaaggaccccgaaaagcatcgtgtgttgttgcaaaggcctgccttatcctggcccccaggtccctctccgctggccctgtctactggataagctgaggttgcacgaagtaggtccaggcctaatgtgacagtgaataatatggtgtttggccacacagagatgtgtgtaggtacaaaaaccaccatgcttttggcggcaaagtaaaaaatgaagatgtcgtcaaacgatctgaactctgatggagactgagcgagagaccctggcccaaaacaatcactccatggcggatgcgctctggggtagacagctactgctctcagagcagctgttttcaggcca

SEQ ID NO: 11 - DNA, 3870 bp, Mus musculus
gtaagagccaattaggaagttccagggttagtaaaggccaatcagtaagcaccagggtaagagccaatcagtaagctccaaggttagtaagagccaatcagtaagctccaggttagtaagaaccaatcggtaagcaccagggttagtaaaggccaatcagtaaactccagggttagcaaagaccaatcaggaagttccagggttagtaatggccaatcagtaagctcctgggttagtaagagcttctggttttggtccttcaatcactggcctgagcactcatgtgattggctaggctggctaatcaaccagctgtgggaatactatccagtgatgggcttgcagacagatgccacagcatgtggcacctttaatgtgggtgctgaggatacaaagtcaggtctctccacgcttgcataggaaacactttaccaaatgagccatttttctcagtttcgattttattttattttttgagacagggtcccactgtatagctcaggttggacacagacttgtgatactcctatcttggcctccttgactactggaattgcaagtgtgtggcaccatgccagctggaaaggtaactttctaaggtacctctttctaaaatagatgttgaccttttgtaaggacagactaaacgccccctgggcttgaggctggcgccatccagaacagggtagagcgtattgagcctggcaggttgaatccatctcccaaatgaagagggcaggtgggttttgggggttgatgacgagggaggggcagaaagagggagacaagacagagagtgttactcagtccaggtactctcttgaactaagagcacacagggaagaagggcctcatctgaggccaaggtgtcattgtatccggtataaggggacaggatcacctcctttcatgttggagctcgtggatcttacattctctaatgcttgactagatgtgagtggagctagaacacgtatcttctcctggtcaccgcccagggttcgtgcgcttttcttactcggtacatcatcctcatcgcagtgggctggtctctggctgcctcatccagtttgtcgtctcagttcatacggacaccccctggcttgtcagtgctggcccagtaccctcgggcctgagcacctgtgatgcccctgcctccagctcttcctccccagagtctgcaatgctatcattccttcccggcccagagacttacgcttcctcattagatgtgggagatgaggttctcaagctccaacaaaccagtcctgacctcgttttggcaggaactcaaagagaagtcagaagcttgctgaatcacccacaccggccggccggccgagcatcctggcaaggcctgtaattagagcctctctttcacaccttgaatcttgagggccccacgtctgaaatgaggggtgtcccagtgcctgctgcaagtttatgagcagcacacagactcctttcctttggaactcaggggtgctgcctgcgtctggcttctgtggaggaggaagtaatgtgtgtggattagtaaaagatcattttcctgctgtttgtcttggcctccgtgcttcagaattcaagcacttgtactcttgaccctgcagtggtggctggttttgagtccacttcctgtctgatcgctaaactgctccttctctgaggaccttcagctgaagccacttacctgctaacacttaattaattaataattaatattgtaattaattttttgttgcaggattggcagtgaaacccaaaacgtcacacatgctaagcaggcacggggccatcaaatcattttcttaattttttacttttttattttttgtgtgtgacagggtctcaagtaacccaggttgaccttaaacttcctgtgtggccagaatggctttgaatctctggcccttcttctccctcccatggtactgagattacaggtatgtaccaccatgcctgacaccctgatgctgtggtggactcaaggaatgcacatacctaagcttgaatgctcgctgttgaaatactagagacatttaaaataatttgccagttaggaaaagctttctatggcacacagtccaattgaatcttaacacacacacacacacacacacacacacacacacacacacacacacaagacttaggtctttcaaattccagcttggtggcttgttccatgtcttctttggacaagccctccagctctcctctcctctgctctcctccttggtaactaaggggaggccacgcctactttattggcatcctagagatgccaacattggcaaagagaagggacaattaaattcattgaggcctgtgtggtgtgtcagcaactctgccaaccactttcttatcttggtatcatttaaattagtttgaacacttaaaaggttgtgtaaatgtggctgtctagtattagaagctgttttgtattattgttagttgtgttccctcaggggaagtgagctgccctgagctcagttctttatctggaaactgggcctaatacctccagactcaaatgactgtcacaggacttagctatgaaggaaagggttgaggcagaagtcagagcactttacaaatattaggcgcacttactaatgctcatgataaattcttcaaattgttgtgcgataaagatcttgtcagggtttctcaggcggctatctttcccatcagagctgtctgtccaagttaaagacagcttactggaatatttctgtatccttttgtccaatacaggatttaaatataccctgcgattagattgtaatgccaataaaaagaaaagaggggatgtcagagcataagcccagggtgacaaccctgggactggcattctagattctggggaggagactctttctgggaagagaggctcatggcgttttgcagtttttgttttctgttttaagacaggagttgctttggggagctttatcttaagaatccgaacggttgtgtaggcaagcaagcaagcaaggcagctactgttcggttgacctcgttctgctgtgaagaatttgcactgtgtgaagtgtgttcaggaaaccctgaatagccttggcacacctccgacgtgctgcttcgtggtaaagtttcctgtcctcaaaagagaagacatttaaaggaagaggagggaccaaagaacgggtcacctagacaacagggatctgggcacctggtaggaaggaaaccttagcttatttactccttgaatgttgggagagaacagccaggaccctgccctagagcctcactcatgaaagctgaatctgggacagtgagtcctcccctctaactgctcccagttccactgtctccagggtggatcccaagtggatgctgtgtacatggccttcattctggtgcctaagctccactctgtggaccctgtcaccaagttggtgtgaggaaatgtaacatttaatattatgggtctgggccacaccaataaactacgaggcattgtagtcaaagctgctgccgcctttcagtcacctgacctcggtggccattgaataagtgaccttggtctaaaacaattgctccaatgttctgttctgatgctctgggtggatcgctgcttgtgtcagagcagatgtttccaggctgttgctggggccaatgtcaccattcctgttagtttcagattgtctattagttctagatagggtctcattatatgagacaccccaccctcctgcatggctcaaaagtttactgatttttattctttgtgtgtaagtgtcttgtgtgcacgcacatatatgtgcaccatatgcattcctggtggtaggaagctagaagaggggctcagattctctggaactggagttacagatagtcgtgagt

SEQ ID NO: 12 - DNA, 1768 bp, Homo sapiens
atcacgcagcccataccctgcggttctccggggacttatgcatcggcccaagttgagggtttgtctgaactgaaacccgcatcctagacctggctttcttctccccaaatccaaggggacaccccggtgacccacaaaagcttagaaaatccaacacgcagcaaatgaaacgggggaaaggggcaccggccctcactctggcctcttagacacacgatatgaaaccttcataaaacctgttgtacaagtcaaaggggaccacgctggggtaaaagtcaaaccagtccatcctcgttcctctgcgtacagagagagggtccagcgcgggcggcgcccactgccatcgggccggggccggggcgcgtggacaggagggtgcggatagaggcagatcgggggcccggtcgccccacgtgcggccagacacccatcccggccgcgctctgccggctctgatccggtgccagacaggagcgacaggggcgaggtggggaccagccgccgacctcacctgttttgttttcttggaggaaattcctccgctggggggccgaggtggcaccgcccgctcgccccccgcaagacccagccggtccgcgcccgcttacctgctctgcggccggcggccctggcgcgggctctgcgcggggcggcgcccttcgctccggctgggcaggcaggtcgggctcgggcgccgccggctgtcgggctctcgtcgggtttcgggtgaaggccccggctcccacctgctgcgccttttaaccgcgccccaccccgcctctgccctgacgcggctcgggcgggctgcgggaggcgagcgctgtcactcgacgagccccccgcccccacctacccggggcgcactagccgctgggcgcggaccgtccccctgaggagcaaggagtgcaggaccggggctgtccctccggggccggatgcgcagagcggggacctttttcccgtggcgggggcgcagggtgggggacccctaagaagtgcacagtgcgcggggccctctttccggcccttggagggaacggggtaccggggatgcagggggtagggctctccctcgggagcgcagagggcgggcccagccccctctgcacgggtgcaggtgtggggcgcctgctcaggccctcgagggaactcttcctccctagtgcacccgtggggagcagtgtgaggggcaggctgtgtttttgccaggacacatcctcagtctttctgggtgatccagccttctcatagcccgcggggtgcacagacctctcctataggagcctggaggttctttattaattaatgaccacttagaggaggtacaggggttgtttttattaattacctccatcctttgaagactcctccggggaagcggagcaggccttcctcgggacagtgcaccaggagagaccacattgcctccccgcttttcagtcaagactagaaagctcagggccagtacagggagtggtgcaagggctggtggggtggaaacgttggaagctatttaggcacctggctttacaggttcaaacctgtcacgcatcggacaaaagatgtgtgacttgcttattctacaaaactgttcggtaattaaacgtccccacctaaaccatatgccacttgttgggtcatattctcccacgaaacaattaagatgtctgttaaaggtcatggaatttgagccaagacttcataaaaatccgctttccaaaatattttatttgaggagaacaaggttcttaaagaatttgcccaagtc

SEQ ID NO: 13 - DNA, 1751 bp, Mus musculus
aatcatgcagcctgaatgggcatttctctccaagtcgcagggtttgactgaccataaacatcattccttgctgtgcttttctgcccgctccccaaatcgatgacagccccaaaccagcaaaggaaatgagaaaagggacttaatccggactctagtcactttaaacagcctggtgtgtttataaaacctgtcgtgcaagtcagaggggcatggtgcatgcagaagtcaaactagtccatcccagttcctactgcagggcacgagggagggggcggcgcgggtgacaaccaccctgccgcggttccagttcccggtgggctcgcaaaggcgggatgccgatgggaggcagataaggatgctggcaaacccccgcctcccccccccccaccccccgcatggtcaagactgtctgtaaccgccgggccgcctggagatacttgccaccccctcgtcccacaaatctggcgagaaagggaacagaccacttcctttacctgcccgggtttctcggaggaaatgctcccactcgcgcttacctgctcggtgggagccggctccaggctcgcagcggcactcagagctcctaccctgagcgtaggttggatcaggcgccggcggttcacagcgggaatggaatcggggacagtgcgggtggagccccggtttccacctgtggcttcttttaaccgcgcccccaccccgcctctgcctgacgccgcacgggagggctgcgggagaggagcgcgggcactcgacgcgccttctgtggtgcgcaccgccctctctccgggacagaggagcggggcgggtccccttctgtggagcaaggggcaggggaccttccctgttagggccaggtcttagtggtactatattagggcactcgttgggatccttcttctgaagccagggaccactgcgagtgtcccctaggagagactccaggtgtaggctggtcttcccttgggttggggacagaaggcttgtcccttcttgtggatgtgggtggagcgtggaccgcgatgggcaagctcagccagatcccatcaaggacagggaaaagttgcccgctggggccttgctggggctggacactggagggcccttaatgaagtgagggctatccagagtacggggaacaggcttgtggacccagctagtagtgagtctctcctgttggtcatcctggtaggaagacaactggtttgttttcatcctttctagaccctttgggcaccctctcctctagagcagcctggaggttctttattccttaatgaccacttaggagtctcaaaggtttgtttttattagtcatctgaatcccttcctgcattgtccagggaaggggagtggacttccatcttgagagatcccactgtgtctgctgtcacatcaagggcagggtaaggtcaaggcaagcatagagggtggtacagggggtcctgggctggaaatgttggaagccatgtaaggacctagttttacagggcctgccctgtgctacttcagacaagacttgtaacatgtgtaacttggttattttacaaaattggctggcaggtatgttcttacctgttgggtcatattctcactttagctacattctacctgttggttcacgttctctcacaaaacgagagtaatagtgcttcctaaaatgtctctcccaggtcatggaggttgagtcaacgctttataaaaacccaccttaataaaatacttgaaccagagttctcggaattggaccc

SEQ ID NO: 14 - DNA, 3358 bp, Homo sapiens
taaaagtgagcaaacagcttgaaccaatctaaacagcttatttatttgaggtaataaacttttccttcttcctgagttttcctaaattcttctctatcatgaaaatagcattaatagctaaaattttaagtgtttagaggttttgcctttcaaatccagtaagtctccagagtcaacaggtgctacaagatgctactggcagtaacagtgcttctccaggattgtggtaggtggtgtctaagggtcttttcagcttgaaggttctgtttcccagttctgtctcacttaagatcagatcttggtgagtatattggcaaaccatttcattatttaaatttgtaaaatacaggctttaggccgggcgcggtggctcacacctgtaatcccagcactttgggaggcccaggcgggcagatcacctgaggttgggagtttgagaccagcctgaccaacatggtgaaactacgtctctactgaaaatacaaacttagccaggcttggtggcacatgcctgtaatcccagctactcgagaggctgaggcaggagaatcgcttgaacccgagaggcggaggttgctgtgagctaagattgtgccattgcactccagcttgggcaacaagaatgaaactccatctcaaaaaaaaaaaaacaacaacaacaacaaaaacaggctttaattgtatttcatactctttaactaactagatattaactataaaatattaacaatttcaaatttttgttaaaggaatacatttacacagcttaaaaattcaagtggaactaaaaggtttacaaggcaatatttcagtcctctgccccattctctgctcctcccaccctgtatgctgtcccagaggcaaccaacgcctttcattttttagagctcttctgacgtttacctttatgtttccaaataatgtgcttattatgccatttactgattgctggactttagacctgttgactttttctgctatggtagtggaggctttagctctgacctgagccccactgctcctgctccacccacacctcttccctcaccctcatgacatgatcatggctcatactctggtcaaatacatattgttatttatattattttgactgcgagcataatgacgtctggaccaagttgtattctatgttacattttcttttggttgcaattgcctcccttccctgagagtgaaccatgactggggttttcatttgcttggctttctatgtgtctattgttcggcttttcctactcttccaacaaatctgtcatatgcccggaaacaattttttcaagttcccagacatggttccgcacagtccatctattccatctgtttctttcccttttcccgggggctgtggtctgggcagggtgctctggccctctgcccagtggtcccctgggctccccttgcctttcccctgggccagagcttgtgctttctggagtccgtgtcttcctgtcttggtctctaccttcattttgctgaagcacacaccttccaggaacttcctcaggaggggaatgtggaactaaacttctatgcacataaagtcttcatatcaccctcaaacccgatctgtctccccgcctccaatgtactttcctttcctctcttattttctctgtttttatgaacttacaccttttttcttcactattgtgtaattggcatttaagatgggagtagagataaatgcacctgtgtaggctcatactaaccacacgcctcagtgcatgggtgtttatcagacttctctcaatcaagagctgcgctgagtacttgtgaaggccctgcagggctggtgctgagtaagttcaggattgggcacctctgaggggtgaggaaatggaggttcagagacgagaaggaacttccccaaggccacatggttaatgattggaagatctgagattctaaaccaaacctgagtcgatcacttccctttctgtccactgcactgataactgaagcccaagggctgaggccacacctcagcgtgtgaggatcagcagaggagaccctgctggctgcgggatgtggataggctttgaggaagaggaaaagcacaggcaaaatgtcaaagataagtgggaatgaggttccctggagcatgagtcgcaggtgctcaggaaggtgctggcagctctagagaaggccagagagaagcacccagtggtgggagccacagccccaagacacaggctaaagccccagcccagggtgggtgagctccaccctgtcacctatggggttgcatgcaagtggttcctctaagcattggcttcatctgggaggcgggggtgacatcgcttctttgagccttatttggaggactaaacaacacatgcattttgtcattaggctggtgcaaaagtaattgtggtttttttctattacttttaatggtaaaaaccgcaattagttttgcagcaacatactaactttaaagttcttaatacatatgagatattatttctatcagcttagaaggatccattatgattgtagaagacctgggatgccagtctgaggaactcttcttttcttaagcaaaggagaaacaaaataattctgatgggggagtgactgaccccagtctggctcaccggcggctgtgaagtcctgagtgtcctctggcagctgcctttgaaagcgcagtggtgtccggggctcgccactgaatagcgtttgttctcagaagggagcccggtggaaaatttgaagctgcagttaggaactgtgtgtatggccttggaaactgaagatgttcctttaaaagaaaaatcacagtgtttttaaaactcagatgacagctttgaccattatctgctttcctctcctgccagctctagagttttcttgggatgttatcaaggatgatatcacaacaatgcccacttctgttttgtttttaacctgaatgacaaattaccaatcagcagatgtaggccatccagggaagtttcttttaaatgctggacttttgcaaaaatgtagagccttggtggcaattgtgattcttttttttttcttttcttttccccaatgaaggtacttttttttatgtccagttttggaaggctcctgaagattgtttgagaacttgactgctgtgtcagggcagtgctgacactctctgttgccaactgttattcattattccaaaaaatcagagaagcaaaaacgacccctccaaacaactccaagacaaactccaagcaaaacaacaacacacacacaaacccacaattttcctttggttgcttctgagaaggagttttaatggtatagtaaatacagcatttatcggatgatttttgctgccattgatatgtttctcttcttg

SEQ ID NO: 15 - DNA, 5018 bp, Mus musculus
aggaggtgtgtcttcctggaggaaatatgtcacaagggtgggctttgagcatttaaaaatttaccccctttccaggtttttctctctgcttcctgcttatggttcaagatacaaactctcagcttccagcttcagcccctctgctctcagagatgctcatctctctggaaccatgggtccaaataaactctttgttctataagttaccatggtcacggtgctttaccacagcaacagcaaagtagctaatataatcttttcaaggccacgaaaaagagaaaggcaaaccaagagtttggctgaccaaatcagctgagaacacaaaccttcccatcctaaattccccaatgttcttttatttttcatcatgcaaatagccactgatatttaaattatattaatgtgctcattatggcagtttcatatatttatatattgtactttgaacatattcacacacctccaaataccctcttctgtcccccacattttaagactggaagtctcgttttttcaaatccattattaggtccttagggtcaatggggtcatatgatggtgtctgtggttctaattagtggccagctggatacctgcagaatcaatgactagtgggtaaaaagtgagcagtcagggtcagcagctcacaaagcgtcagtgagaggcggacaaagagagctttcagcaacccctaactgggtgggcagcatgtgagccaagtgtgagtccctcctttttggacctgggagaccagcagagtgtgcaggccctccgttggcttggcccaggtgataagctgacctcagcaggaattacctcagtcttagtccagctcctgatgtaagtctcactcaaaacaaaacaaacaagcctagacaaaaccagcttgttgtcttttttctgttgtgggaactgctcccactcaggaatttctcagtggccccctcaaggaagtttgcttcttctctgcttccttccacacatctgtgtctttctggttggagaccatggacttgagagttcaagttgagcttccactaccctaagtgcctgggtcaagcacacctgcgctgagaagggtcctgccagtctcaaaactgcatcactagatcagcagtatactctctcacttaagcatggagtggggaggtgcctttgtatgtcttagcaatagtcatctacgtgattttgaggtcattttacttttaaagtatataatcttcaaaccaaattcaaagactaggcaaaatttttaaattagcttttaaaaaatgagctggtttgcttacttccctgatcttaattcctataggcagtattgtgaggtaacttatttaggtttagggatgatagagaaataatgtcttagggttttactcctgtgaacagacactatgaccaaggcaacacttataaagacaatgtttaattggggctggcttacaggttcagttgttcagtccattatcaaggcaggaacatggcagtgtctaggcaggtatggtgcaggaggagctgagagttctacagcttcatctgaaggaagctacgagaatcctggcttctaggaagctaggatgaggatcttaaagcccacgctcacagtgacacacttcttccaacaaggccacacctccaaatagtgccactccttgggccaagcatattcaaatcactatgggtactcttaaaagaatgcatgttttagctttaaacattgttcatttatccgtgtaacagactggtttgagatctctcagcaaagggagttatccttatacagggactcttttcattctttttcttagtgcatattcattgtagatagtgctgagttgtataaaggctttatctatctatctatctatctatctatctacatcccaaatgttgcccccctccccgtaccccctcaaagagttctttctcccacccccattctctttgcctttaagaggcaacctcctcttatatctccccaacctgatgcatcaaatctctgcaggattaggcctcaggccagcccatgtatgctctttggttggtgactcagtctctggaagctcccaggggtccaggttagttgacactgttggttttcttgtggggttgccatctgcttgagggccttcaatccttcccctaactctcccacaggggttcccaacctccagtcagtccagtgtttatctatgggtatctggatatccccctctgtctcatcagctgctgggtacagcctctcagaggcctgctatgctaggctcctgtctgcaagcacaacatagtatcatcaatggtgtgagtgatgggtgcctgcccatgggatgggtctcaaaacgatctgatcactggtcagccattccttcagtctttgctccatctttgtccctgcctttcttttagacaagatcaatttggggtcaaattataaaggcattttcatgttaagtgtataatgtattttgaccatgtttccccatatcctcctaccctcccatttgccctccccctttctcattagtattctttgttctagacaaatttactctacttttatggcatatgacacatacatgatttaatgaaacataaaatggagaatctacagacaaaagaaagcatgaaatatttggctgaagctgactcaactcatttaatatgacaacctccatttccctacaaataagagaatctcattctttattgcagactaaaattccacaggtgtatataccacatttctttccctatccctctgtctttggacacctaggcaggttccaccgtgtagctattgtgagtaatgctgtagtcaacattgacatgcaagtgtctctgtgacatgttgacacagagttctctggataaacacataggagtgtcgtagctgaatggcagtcgattgagaaaacaaataataaaagggttggtgagcaggtgggaaaaggaaactttgaacgcattgctggtgagaaggaaagtcagtctagctgctatggaaatcagggcgagggttcctcaggccctaaaaccagaactgccttatgacccaggcagtcttgacagctgttgttgtctgtgcttaagttcttgactctgtcagacatagagaaaccagatctcaggctagaagttccttctttctccatgttcccttaaccaccctcttctctcctgcctcagccttgtagaagtgtgccttccattaggcacctaagaagaggaacttgacagtcagctgccaccttctagtgactggaagaaccaaatattctggatctgaataaaagattttacattctgctttgtggctcacaggagactcagtgacaggcccacctaagcacacacagaacagtagagcgacaggttgaaacagcttccaggaggagtggggggaggacgggctgaggaagtgggatgtgtaattccagtagagaaagtcattggaggtacggaaggtgctggcaaccctgagaaacagcagctgatccaccagctgcagggccaggcctctggatgcaacagccaagtcagagcccagctgggcctggctgtgttccacctgctccctgggtggccccaggcaagtgactcccctgagaactggcttcagtagtgagaagaggggtggggtgacaatagcctctttacagggttacctagaggactaaataatgcacatacgcatacacacacacagacatgcacacatagacgcacacatagacacatagacacagacacacacacagaaacagacactgacacacacatacacatacacaaagacacacagaaacagacacatacatatatgtatacacacagagatatacaaatatacatacacacatggacacaaacacacacatacagaaacagacacacagacacacacaccaacatataatacacacccatataacacacacatataacacacacacacaggcaaacacatgggtttatgggctctgcagtacaataaggctttattttcatcagcttagtcagcagtagcctacaaatattagtgttcaaaagtattttctaggcaagggagagacagaaagtggttgtggtggggagtgaggctggtgactgtgagtgggcagtgtctagtgtctggggacagctgagattggcagcccactggccactgactagagttgcttcccacaagtgagtccagtggaaatttttagtttgctcttagaaactgtgccttcagccttggaaactgaagatgtttctttaaaagaaaaatcgtgctttttgaaactcaaatgagagcattgcctgcggtctgcttttctctctctctctctcaccagttttcctgggatgttatcagggccaatcatcagaacaatgctcacttctatcttgtgtctaacctggatgacaaatggccagtcagccgatgtaggtcacgcaaggaagtctgtctttcgggttggactgaggtagccgcagtgcgatggctgctttgttgtttctttcccttttcttgtcccaactaaaagcgcttctggtctgggagtaggggcgactgaaggctgtttgagaacttgactgctgggcccctctaacattttctgttgccaacagcttactccttttgctaaaaaaaaaaaaaaaaaaaaaaaaagcaaacaagcccaaactacttcttcaaacaattctaagacaccacacaaacagaacagactgaagccccagtaacccagctttcccagggatgtttgtgagaaccagggtagtttttgatcactactaaattctacttaaacatttttaaaggatttctttttcttctcgtttttaaatttgttcttcgaatacaatgtatttttgatcatatgtgcacccctcccccaacccctccttctatcaagccaacctggtgttccctcccctcccctctccctcctcctctccctcccctccctctctccttccctttccctcatctccccctccccttcccctcatttccccctccccttccc

SEQ ID NO: 16 - DNA, 5079 bp, Homo sapiens
gttttaatggtatagtaaatacagcatttatcggatgatttttgctgccattgatatgtttctcttcttgaaagaggaattcaaatgacaatgaacatttttggggtcctcttttatggagtttgattttcaggggattgtcaggcatgtcgtctccgggttcccatgctgcacagtcccagcactctctgtggctcagccttcccgtcccttgccctctgaataccttgccgttgactgaatggtcatcgttagcacaggtcatcacaatacatgactcctgggcaggaggaacagaggagcggaggttgtgccatgcatttaaaacccagttagcatcccagtgggtcttccaaggccgaagatggcaaaacgtttttattttactttgttgaaatcatctgtttccctccaaatggtgggctgtttgggcacaaggtcatgttgtcttcaatttcatagccccggtacccagcaaggatggctgcccataggctctattaagatgccgagtgcatccgtggcacggccaggaggagtgtgctgtggtcagccttccagaaggaatcaatctcctgggagaagtggagaagttggcctgcagcaggggcctcgagaatggcgggtctcatccaccaccagcaggctcgtctgttgcccagcagtgtgatcctagctgaggtttattctctttccctcattagactgcagtctcctgaaaggcagggtgtgcacctgacttgtctttttgtcccttcatcctgcgccctgcacggtttgatcagtaaatggtggctgagagacaagggagtgggaaggaaggaggtcaggaggggagagaggtctgagtgcttgaaagagtccctcctctgcttcaggggcttgttctggggttttctggatcttcagtacttgcgggtaggatctgagctctcccggcccctggtggttgttggccaggcctggccagcttccagcagcacaggtcatcataatatatgactcctggacaggaggaacagaggagcggaggtcgtgccatgcatttaaaacccagttagcatcccactgggtcttccaaggcggaagatggcaaaacgtttttattttactttgttgaaatgcaggttgttcctttttttttaaccaacttttatgttccaaggctaaaacatagcataaaacaatttgaaaaagtcggtttcaatgtttcccattgttcactgagagagggtcacacagggtgcaaggcaacagaggacaccattgcttacgtagtacctcgtgagctgcactgcgagaggcctttcaaaggaaggttttatttaggaagcaaggaatgattaaaaactgatggctctaatcaaatgagatttaaaattttccattaaaccttcatagttaggctgcatgcagtggctcatgcttgtaactccagcactttgggaggctgagatgggaggatcacttgaggccaggaggttgaggctgcagtgagctgtgactggggcactgcacttcagtctgagtgacagagggagactgtatctcaaaaaataaaaaaaattaaaaattaaaagaaataaacctttaacattgggtgtaattttactttccatctactccttcttcctcacctgcaacgttcaagagcaggagggaagatgtgaacacacatttgtgtgtgtgtgtaaacatgctcatgtgtttctaaattatcaagtcaggataagaacttctactgtgaaatacagatatacaacaatatgtcccaagctatgtttaatgcacttttattatcctgctagttcttctaaatatgatcattatacaatagttctttttttttttttttttgagatggagtcttgctctgtcacctaggctggagtgcagtagcgcaatctcggctcactgcaacctccgccccccagattcaagcaattatcctgactcagcctcccgagtagctgggactacaggcgcgtgccaccacacccagctaatttttgtatttttagtagagacgggggtcttgcctcgtgggccagtttggtctcgaactcctgacctcaggtgatccacccaccttggcctcccaaagtgctaggattacaggtgtgagccactgtgcccggcccattatacaatagttctacaaagaaaatttaagagcaagctctggcttagtctttgaaaaacaagtttggaatttcctatacgagtggataaaatgtcagctcttggtattgtccttaagacacagtacatggtatttactctctttttatagggtaaagatagataaatccccaaaggccttggcatttaggaaacaatcatgctttatctattaacttactctttaagctctgtcattttttgcgtctgagtgagacactctatttactgagccacagaccacctgctagataagcagagactcttccagggcacacagcctggagaaaaaacgcctgaatgcacaactagaagtattagcaagtctggtttaactgtccccaaatgtctaactaagaatattagtgggccaggcgcagtggctcacgcctgtaatcccagcactttgggaggccgaggcgggcggatcatgaggtcaggagatcgagaccatcctggctaacacagtgaaaccccatctctactgaaaatacaaaaaaattagctggacatggtggcagccacctgctctagtcccagctactcgggaggctgaggcaggagaatggcatgaacccgggaggcggagcttgcagtgagccgagcccgcgccactgcactccagcctgggcgatagagcgagactctgcctcaaaaaaaaaaaaagaatattagtgaatgattagtatatgggaaacacctccggaccaccctacattattattagtcttcactttgtggtgggtaaagataaaataaaagtagctaccgtttattgaatgtttaccatgtgtggatgaaaaccatgttaatcattgtcttctttaatcctcacagcaacctaatgaagtaggtactataattttgcagatagccacattgagggtgagtgaggttaaacaacttgctcatatgactcaaaagtttggaagccattttcaaatcagatgtggacaaagtgtgcctttttaaccattgtattattcagtcttcctatgaagacacgcctctatttggggcatttacttcctatataacttgatgaaaaaaaacccagcattttcattgcttgcctataaaaactctaaaggtgtttctgtgggagggtgtgttattccactcagctattgataaatatagtcctgtcttaatgtttaatgtggatcttttttctgtttcatgcttttctgaatttttgagtgaccatgtcactcagaaaagctttgaatcagcaacatttccagtggactgtagggaaagcctgttgttttggtggaaagtagagagtcacagatccccaaccttcatctgagccgtggttctgcatcagtacagacaggaaaccaactattaggagccactacatgaaatagtatttcctcaggtgagcaaaaaattcttttgcttttgtagattggccctgtctatacgtggtagccactagtcacatgtggcttttgacgtttgcattttaattaattaaagtgaaacacaatttaaagttcagtcacccctgccacactataagtgcccagtattcaatacaactgcccagtggctgccatgctgggcggcgcaaacgtagagcacttctgtcctggctgaaaattctactagacagagccatccaggaatttggactagcaagcaccaagttcacagttagagaacacagttgcaggccaggcgcggtggctcacgcctgtaatcccagcactttgggaggccaaggcggatggatcacgaaatcaggagtttgagaccagcctggccagcacggtgaaaccccatctctactaaaaatacaaaaaattagccaggcatggtggtgctcacctgtaatcccagctactcgggaggctgaggcagaagaatcacttgaacccaggaggcggaggttgcagtgagctgagattgcgtcactgcactccagcctgggcaatagagcaagactctgtctcaaaaaaaaaaaaaaaaaaaaaaaaaaggaaagaaaaagaaaaaagagaagacagctgctttacaaagcaagagggcttcaagaatctggaaaccaaaggagcaatgtcctttgagtttctacaaatttgggccacactgattgggcctttccacagccaattccatttgccttcattatggaaagtaaacagtttaacttcctactgacatgctctgcagtgcagacagtaaacagtagctcaccgctgcttctgccagctgctctcgggtgttctacttgggtggggaacagcagcactggcactggcactggccccggtggccccacagagcatggctccatcaggctgggtgctacagagggatgccaagaacatttgggcattgaatgcctctctctctctctctctctgaaatgaaaaccctcatcaattcaacaatagtttctctaatagaacatatagtgatttgtttcatctcaactgttcccatacaataatagaaaggagggagtctgtgcctgagagtgcctgcaaaccccagggcacaccagccccgtggagccataacagttgctcacagagacagcccctcacagcagcccccggcacagtgactcgtgtaatgaaagctggaaaattgcccaggaaaacctgaagatgcattcctgaagctcccacactccaacgcacgcacacacagacttctctcctggctttaggaacatgaatttaccttgaatctttaaacttaattgaaaatcttgcaaaataacgagctttcctttgaatcttcatggcactttgtaataaaatgtctaaaagggggccattccatgaaatcatttaattggcattaatagtacactattacttcatataaaatcataatcatataaatgtacttatataactccatgtaaattaatttatataa

SEQ ID NO: 17 - DNA, 4077 bp, Mus musculus
gggtagtttttgatcactactaaattctacttaaacatttttaaaggatttctttttcttctcgtttttaaatttgttcttcgaatacaatgtatttttgatcatatgtgcacccctcccccaacccctccttctatcaagccaacctggtgttccctcccctcccctctccctcctcctctccctcccctccctctctccttccctttccctcatctccccctccccttcccctcatttccccctccccttcccctccctcctccttcccctccctttctctcccctcctttacctcccctctcttccccttccccctccctccctcccttcctccttcttctggaggttatggtagcactaggagtcaaatccagagcctgacactcaactgctgattgaacccctgacccttcttattttttctgtccatgtttattttcttgaaggaggaattacataaaaaatgagcctttcggaggtcttccttccttgagtctgctgttagggatgagtcccgtttgaatttctgtccatggcagggtctagcgccgatttctctctgatccccagaacctcaccctgatgaggtttgtgcgatgggtgacactaaacagtgttttctactaaacagtgggctttgtggggacagggtgacactgtcttccacttgctctgagttccccgcaggcatcacccccttcctccccactggtgccccactctctctatctgggtaggttgcaggccccctcacagttctacctggaacgtgctgtggtcagcgcaggcaggagctggctggcctttgtaagactggccaactagagcgatgcaaagccggcctggcaccaacccgggctgctctgcagaaagctagctgatttccagcctgagcaggtgcctgtgactccaggggcagggtctctgtcagacgcacctctatccatccttcatcttatccctatgttctgactgttaaatggcaactgagtgaggaggggaaggaaggcagaggaggggtctgagagggatttgagtgttcccaggcccttgcagaggctgtcccgggtctggagggcttcagccagggtgtcctatgtaacacaggatcctcagatagcaggtactgttaaagaggaggccatcacacctgtgcatttgagaccatgccaaagcaaaaggtgtcaacacccgcattttactgcatggaaatgtagttcgttccttttcaaccttttgtatcgtggggctgaagagatgatgtgaaaggactttaaaaactccactaggcttctctgctttgttcactgtagaaggtcacagggagttcaagaaaacaggctagggataggaggatgctcatgtgcttctcttgtgagcggtggcagggccagctccgtctcaaagcaggctttatctagaaactggtgaggtggcaggagcttaggaggagggagaaattgatttaaatattttcattaaacactccctcactgatggtaatttcacttgctctctccctcttagccccccacacttcagaacaggagagagaggatactcgcatacacacacatttaagtgcaggcacacacatagatatgtatttctaaaccatttttcctgtgaatacaatgatgtgctccgatatatacttaagccagtcttactattaaaccatctcttctaaaaaatatgatcaaaacacagttgttctaaaagcaaactctaaaagactgacctagtctctgacaatgagtttgaaaaagtgcagctcttggtgttgtctgcaaacccaacactatttgttgacttgacaggcaagacagacaaaccctcaaagttaatggtttctctattcgtttactctgtaagtgctctctgcattcaagcgagatactgcattggctgacacattaaatatgctgagactcttccagaacgcagcaggcagacaacccacggtcaacagtgggggaatggtatttgtctggcttagttatctccaaatgtctagagagagaataatagtatataatggtgcatggaaaacacccatgagccttggtgtgttattagtagtagttactttatagtgggtaatgacaaaataaaggtagcttccagtttctgaaggtttactatgtgtggatgtaacccttgctaatcaccaccttagttaatccaaacaacagtcccatgaagtatgactattattatccccattttacagacaaacaaaatgaggactacagaggttaataacttgccccaagtcatggtaccaaagggtttgggagccattatttcagtcaaattctaaccaagtgtgcttagccatcgtgccagaggttccaaggaaggagtttgcttgtttgttttatttatatcacttgatgaaataaaactaccattcccattacatataaaacctcctatagatgcctccttagcatgctgtgtgattccactaagctgttgatagacacagtcctcggggctgggggtgtgggtcatttgttagcatgcatgaggtcttgggtttgatccccagcactgataaagctggcatggtgatgtatgcctgtcaccccaggacttcagagatggaggaagccattcagtgccatcaccagctacataatgagtaagaaagagaccagcctggaacacatggcattttatcttaaaaaaaaaaaaagacattcgttttgacatgtatattttttgcttttgtaaattttcaagggaatgtttcacccagaagctttgcactgctgatggtacacgtctgaaatgtcagcaatccagaggctgaggcaggaggattattgagttccaggtcagctgggtctaaacacaggaggaaagtagagctttgagtggacaccatgttcagatgctcaatgatcttcagagttatgcttttggcagacaccacaccaacagaaaaacaagaacaacaattgccttcaaagggagggcagccttgtgaagctctgattcaaaggagaattgtcctttggagtctgaatgaatttggaccgctctttctgagcctttccaattctactggcatccacaactgaaaacaaacagcggtgccctgattgccacagacactctctgctgggcagacagcacaccgcagttcccaggctgttctgccagcatctctcaggtgttcagcctgggtggggaattgcaacatgtgtagcaagccaggtggccctgcagagcctgtctccaacttcgatgctgctggggacacaaagaacattagggcatggagtggctctgtcagtctctgtgagggaagcccttgctcaccacataacatcattccctaggtgtgttcctgcacatatcctaatttgttttaactctgtatttatagtgagaattgttaagagaatcttaggactgagcaggactgaaccagacagagacagcagttccatgttgccagacagatcttacacaggcttagcctggtcgcagccaccagaccaggtccctgttcagtgagaggtggaaagaaatacacatggattttttttttcattttttgctttgtaaatcatgtgggagatggaaaagtttacacatagattttttttttcttttcgttatttgttttataagtcattactcactagcctaggctagcttggagcactctctgtagctcaggctggccttgaactcttagcatctcagcttcagcctcctgagaactgggattacatagctatgatactatacctggcgcccagatgtgtttaaaagcctcaacttcccaatagacctagacgctcctttctcagtctgaaggacacaaatgtacctcaatctacaaacttaatcacaaatctctcaagggtgtttctgaaacttcagagcactttggaacaaactttcctagtggggaggtttgtttcttcactcatttaactggcaaagtcacaactatacaacttcatttatttatataattctatctaactaatggaaataagaggtgaggttagagaagaggaataacttttaatattctgtagtaaagtagtgaag

SEQ ID NO: 18 - DNA, 1501 bp, Mus musculus
gacttgcagtcttcaagaacggatgatgccccaggcaaaaggggtatcctaccctgccacttagtgggccccaaaggagaggcttctgctctagggcaaagcttcatttccctcttcctttgagctcacttatttggaatgagtatgtctgccccttgcctgccctatcatggtcttttgggaacacacaacaaacctggttttgccggttcacagccagaggacggattcccttctacatgggtctgcctataccagatgatgtgatactgtgttgacttgggacttggagtggtttgggcatgggttaagactttgggccagttgggatggggtaagtgcgtttagcatgtgaggatgctaaatatgaacttgggggacatagagaatatggagttatagacccagtggtatccttccagatttgtaattaaatctgtacagttcaatacctcaaaatgtgactatatttggagacagggcttccatggggagatgacattgaaatggggccgtcaggatggactctaacctgaatgatgtctttgtaagagaatcattagctacaaagagagcccaggggcacacacttagaaaggatcccacaaggacacaggaagggagtggacatgtgcaaggcaggcagaggcctcctgagaaatcggttctgtctgcaccttgatcttggatatccagcctctagaattatgaatgcattgccttctttgacaaatctgtatctaaaagaaaggagggtgttatttgttttagctcaagttctagtacaaggtcacttggccccttgtgcttgggtggagcatcataacatttggcagaagacagccattcgtgtcataggagataggatgcagaggacaagtggaaggggaggggactggacacataggcacaacacccgtggtgacctgcttaccccagctgggccgatacctcctgagattccagcaccatccaaaacagcaccatgagcaggagaacagatttgagagccattatgcatgcaagccataacagtgagggaatacatttctgctaagtcataagtaatactgacttcaatcttaaaatcccagggaagctgatgaagctcagcggtaaggcacttgctggcgtgctagaggctctgggttcccatccctcccagacaatttaccagagtcttcccttggtgttagcagttttgggtcctcttgtcttcacattaaaactgacattcacatggaatgatttttgctaatggtgagaaagggttcattttattctcattaagagggtcaactaagtaccacacacacacacacacacacacacacacacacaccccacagattatttgcagcccctcggtcttaagtgatgcaattgctgtgcactcctgtcttgcaggctgtgctctgttctattggtggttcaccagcctgtgccaacactgactggaagaacaagctctctctggttcatcttcacagtcttggttatt

SEQ ID NO: 19 - DNA, 1909 bp, Homo sapiens
gaatgtttacatgtacatttcaaacccagttttctaattgtgcagtcttaatttcctagttaatttcactttacagataagaagctctggagacatggcctttccggttaaagacacagagcccaggcactgcccacggcttcctccacactcatgctgctttcccttaggtaagacaaacctcaccaaagctgagactggctcaagaaacggggaagcctaatgcttgtaaacattcccttaattggaagcattaggcaccaaaattcttcctaaaaaatatgtaagccccaagaatgaaagggccatggttagcacaaaccgcacctcctgagcccagcaaaacccaacaggcacagtgcagcacagcctgggcggtctctcaggtgagtctctgcctcgctcttgccctgtctgtcacctcatctctgccaagtctgaaaatcctgagctccagggactgtgggaacttcactagacatgtgtgaacaactctacattctgatccgtagcgtctccctaatgatgcacatctaggaaggagagggagggagagggagcgtgtgcattccttggagcaacgaggacagcctagtgatttgcaaactctttgcggcctcctggtgggcttcagaatcaatttgtgagtcccaaccagaattttctacataattagaataaaacagagttaagatatgagtgcatcgtatgttgcaagatactgttttgtaaacgttgtttcagatatttgtgagtgcacatgtgtgtgtgcagtaatgggtcacaaaatatatttactctgggtcatgttttaagagggctagaaggcaacactaacataggatggttggaagatggtcaggctcagaacatcagattttgcctccttccagggtaccacttttatcaagtcacacattccttcccgctctgcttttgtgtttctcaatcgctatccaaatttgcgcagaagtcaggaatcacgtgggtaaagatttaagctgtacttctgtgttaattaagcacgttgaagaagaggtgctctgggggaacgtggagaaggtgggtagcgagggctccaggggctcagaaggtggcctcgaggggctctcatctgccatccttgtgagggagaaagtcctaaaccagtcgtaacattgccagaacaaggggtcccaatccagacctccaaagagggtgcttggatctctcatgggaaggaattcaaggtgagtcacaaagtgctgtgagaagagagagttttttggaagttacgcagatacagagtagggtgtcctcagaaagcaagaggaggaactgcctcgtctttaagtttttcttacataggagtcctctctatgtaaagacagagctaagctgtgtctctatgtgggtgggctgacagcgtgacaaaatttattattctgttgatttaaagaaaactatactcaatattttaatgtgtaagtacatcaagtcataattataattatcttgaaagcatatattgttatgggtattgggacctctggacttttcgttgtcatatgattgtatccttgcaggtatctttaggctgtttcttcaactgtaaatatcttatgactgtgggtcgtgaccggcaaggaatggagttggtttttaaaatggtgtcaccctggctcttctatgctcctgtttccctaacagtaatagcccagccattctctcccatgttctcctctgccctcaacttcagaatgaagtcaatttttatttcagccaaaataggaggattctattctgtctgttgaggtctgctgtggtctaatgatgttaataaccagtggctgggcatgattacacgacgaggattctaaatcctgtttcatgtttccctctgggcccactggctatatgaccccttaaa

SEQ ID NO: 20 - DNA, 1201 bp, Mus musculus
gagtatatatgtttctaagccaggttcctaactatgtagtattaatttcctaatgaaacaccctttacaggtagtgaggcctttggagaccagggctttaaaggccaagtagctgaagcccagggtctttccatggcttcttcctatgactgtttatctaatagatgagacaaaccttttcaaaactgattatcagttaagttccaagaaagcaccactgtaaatgttaatgttcctttgaaatggaagtatttagcgctctgtgtgtgtgtgtgagtgtgtgtgtgttgtgcagttgggtacatatatgcagatatgcacaattgtttgtgtttgtgggtctttgtgtgtgtgtgcaggtctaaagtttttcttttcattagttatggtctaaagtggttttaaaaaaagaaaaagaagagcagagaaggctatgatagcatgaggttcctttgggattgtctggcttagaacgctaggttttcccatgttttaacagcttcccatgtccttcccactctgcctttgtctttctcattgtgatccagatttgccccagagggggagaacccagtaggtaagagttcacgctgtacttccatgttaattaagtgatgtggaagtcttggaaaggctgggcagtttttcctgtcttcccaggagctgggggaggttcatccttaatggaaccagttccatgccatccccaggaggcaagaagtctggaaacatcaataattattcagtcacaacaacccactttcctctctccccctaatcctcaactgctgacttcaggacaaagtccatctgatttcaatcagataggaagactagttagaggcctgccccagtttactggctgcagcaacaggaagcacaggttacaataccaagtgattccacgctgaaagcttcactctgatcatcctaccaggctgctacatgagcccttgaaagcgaattatccccggagacttactttctatataacacatatatacttacatatacatgtcgactttgttttttcttgtatgctgtaaagatgcctaggatacatttaaggatgcaacataaaagtcactttcttcatggagtaattattataatagtacttgtttctgggggagcaaattgaaatgtttcccagtgtgaactgccaagttaaaacaacaaaaagctagttggagctccccct

SEQ ID NO: 21 - DNA, 3995 bp, Homo sapiens
ctaacatagggtcgttagtgtcagaactgaattaaattgtaggacatgcaggtggtgactgcagagaattggagcattgcttggagtgaaaaccaagcccacatatttggtgtcaaaagtgttatacaagtagaaaaacaggttctctttaatggaatattattcagccgtattaaggaatgaggttcagacccatactacagcacatatgaatctccaaaatattgtgtttagtgaaataatatagacacaaaggacaaatactgtataattgcacttacatgaggtgcctggaataggcaaatccatagagacaggcagtagaatcatggttgccaggggctgggcgggagggagaatggagagttagtgcttaatgggtacagagtttctgtttagaggtgatgaaaacagtttggaaatagtggtgatgattgtactatattgtgaatgtatgtaatgccactcaccgaacactctaaagtgtttgaaatagcaaatttctattatacgtattttaccatagtttttaagttaattaccatagtttttaaaagttaataggataatattccctgaaccactatacactttagattggtacactgtgtggcatgtgcattatatctcaatgaagttgttaaaaacaagatttaaaagcagagattgggtaaagtaaaggtttgctctgtgctgagctgtgtggcatgtggacctgttttcccaggagggagcactcctggggttttggccgcagctgcacatcagccccctgtgcagaggaggtatggtgtgtgatctggagattagctgtttctagtgcagtatttacatttaaagacattgctgagttaggcagaattttctatatccatttgtattttgcttggcattcactttcttacaaaaatggacaatcaagacaaagaaaacaaaaggtccaattactactcttcatttcaccccaaagcaaaacaatattagttttcaatttttttttcccatagaaagcaataacagtcccatactacctcctcttccatgaaagtagtgcttgagatgccccaaggaaaaaccattctttccaaagatgaaagactttgtacctgtcaggtgaagagatggaataaatgccactcctagtgggtgtgggacttgtgcagcccctggtccccagttatctgcttatcagaatgtggtttgcatatcacctttagcggaattccttgggatgcttgtaattctgggggagatgtctggagtctgcatttttagccagtactcctatgacttaggcacagtagggaaccactggtgccattccttccttcctttcttccttccttccttccttccttctttccttccttccttccttcctccctccctccgtccttccctccctccttctttctctctttctttctttcttcggagtctcactctgtcacccaagctggattgcaatggtgtgatcttggctcactgcaacctctgtcttctgggttcaagtgattctcctgcctcagcctgctcagtagctggtattataggtgtgcaccaccacacccagctaatttttttggattttagtggaggggtttcaccacgttgagcaggctgatcttgaactcctggcttcaaatgatccacccgcctcagcctcccaaagtacttggattacaggcgtgaaccactgcgccctgctgcaatgcttttgctttccgtatacaaggaggggttgcaggcttgactctaaaatgattgactttatggaggaccgtctcatgtctggatggtaagtgataggggagggggcaaccctaaatgggatcccaatgacttgatgaaagactggaagatgagacactttcaggtgtgcataatggaagacttacgtaggactaggaccaagcctctcaattatactaagttgtccatgattgaccagggatttgatgaaaatcccactgccttcctagaaaggttaagagaggccttggtaaagcacacctctctatctcctgattcagtcaagggacagctaatcctaaaggatgaatttggctgggcatggtggctcatgcgtgtaatcccagcactttgggaggctgaggtgggaggatcacctgaggtcaagagtttgagaccagccttgtcaacgtggtgaaaccctgtctctactaaaaatacaaaaaaaattagctgggtgtggtggcaggtgcctgtaatctcagctactcgggaggtggaggcaggagaattgtttgaatctgggaggcagaggtttgcagggaacctagatcgcaccattgcactccaacctgggtgacaagcaaaactccatctcaaaaaaataaaagggataaatttattactcaagctgcccgatatcaggaggaagttgcagaaaggggccctgggtccagaaagtacattagaggacctcctgaaaatggccaccttggtcttttatgattgagacagggaggcctgggaaagagagaggagatacaggtattccagggtgcacctgttaacttctaaagatatggcaagaacagttctctctcttctaaagtttatctgcccccgtacaaggtttaatttctttcaccagggtgaaacagcttggagtacaatgttgttgttagtatatttcacttatctctgttggcactaaattctttccttgtataatacacatgtttaacttatgcatacttgaccttataaaacttgtttttttctctcatgcctagaagccatcaaactccaaatggtcaggcaactggagcctcagatgatagctcccctttgctaggaacccttaaatagacctctgggaggactctgactgccattttctccaaaacaacaccccttgtcagcaggaagcagcaagactggtcatcaaccatattctaacggcagtattcctatgatttagccagtgggccgtgaccggcaaggaatgtgccttgttagtttcaagatggagttgatttttaaaatcatgtcaccctggctcttctatgctcctgttcccctaacagtaatagcccagccattctctgccatgttttcctctgcccccagcttccgaatgaagtcaatttttatttcttcaacgtacctcttcagaggggaaattatacaggaggggggcagggaagtgctgggtagagaaaggtggatccccagctagggttccacccccacagacctaggtgaggaaaggcacttctggcttcacacccaaatgttgcattttcgaagaccaacctggcctgccatgcccccattctgggcctataaaaacccaccaccctagcggacagacacacaggtggccagacgtcaagaacagcacatcagcagttgaagacacaaaagggtggacgacaagaaggcatcacaagagaacgtcaagggagcacgccgatggaagaacctgctggcaggctatccactgttggcatgagggggagtttggctggggcagtcagagaagagcccggctgcatagcggcccaattccaggggaaaaccatctctcttttggctcccccggcagagagctacttctgctcaataaaacttggcttttattcaccaagcccaggtgtgatccgattcttccggtacaccaaagcaagaatccctctgtccttgtgacaaggtagagggtctaattgagctggttaatacaagccacctatagagagcaaactaagaaagcaccctgtaacacaggcccactggggcttcaggagctgtaaacattcacccctagacactgccgtggggtcggagccccccagcctgcctatctgtatgctcccctagaggtttgtgcagtgaggcactgaggaagtgagccatactcccatccacgccctacaaaggggataagggaatctttcctgtttcataagtagcaatctctgtggtaacagcccctgtggtgatgccgtctctctcggttctgccct

SEQ ID NO: 22 - DNA, 1651 bp, Mus musculus
tccttggctactttctctagctcctccattgggagccctatgatccatccattagctgactgatgacactgcattctttaatatatggggtttgcactaacttggggtagttattgtcatgtttgaactaaattataggacctccagttgctggagaattgctctgtgtggactgtccacacatatttggtttctaaaatgtcatataagcagacactgcagtttctccacagtggaatcttacccgggcataataagggaagacattcggcacaagcttcaacacaggtgaaccttagaaaacatgctagtgaaataatccacaccccaaaggacaaacaggaaatgattcttatacaagacacctggcagaggccagcttaaagagacaggcagaagatgtgagtcccaaggactgcggaggggaaatgacagccagtgttttgtgggtgctgagggcaacagtttggagtagacaatggtgatgcagggctgtgaacgggctcagtgccgctcactgaaccaaacagcctaagtgtttataataacaaaagtaatactgacatacaccttccgttgtttgaaagagttaataaggtaacattccccaaatcactttaaacaggcaaactatgtgaaatataaatctgtttctgtgaagctgcttttttaaatgcttctcctatcagaggtcagaagaaagaaggcttgctgggagtggagttggctgtgtatctcagacctgtttttgcaggaggagtgtgcgctccgggatttggcagcggctcgagtcatccctgtgagaggcaggcatggtgcgtgatcctggggcttttctgtttctagtgttctatttattttaaagacattgctgagttcagcagaaatgtttcacatccatttgtattttccttggtactcatttccttacaaaaatgacgatcaaagcaaagaaaacagagaatcttcattttaccccaaagcaaagtgagtgcacttctaataccataacagaaaaaacgcttcgggcccttaggaagtgctgaagaagctgggcaaggtggtgggtgcctttagacccaaaggaaagtgattttctccaaatgtgagaggcctgcgatgatggggtgagtggcccccagaggatgtggggactgactagcgctgtctccgtctgtatgcccagtgaagctgtgggtgggacacaattaacagcacaagtctgagtggtgagaccctctgctgtgacgaaccctgcactgatgttactgttgaaggtatctctcaagtgctcatgctggaaactaagcccccagtttctagttgatgttgtttggaggtgggatcttatgggaggggattaggattagatgatgtcataggggtggggcctccacaatggcattaattgctttagaggaagcagacaagaccaaactagcacatttacgctgtcttaccgtgagagtaatctgccatcttctgaggcaggtgagttgatatcaccagatgcccacaccatgcatttgggctccacagtctccagaatcataggttttgaacctttattctttataagttttctagactggggcattctgttacagcagcaagaactagactaatatacatccctccttccatctgccca

SEQ ID NO: 23 - DNA, 751 bp, Mus musculus
tgtgtgcaccagctttgactgctgctggaggctgcccatttcctgtgatctcaaccagcttttctgataggccagtttatctctggactctggcctatgcctgatacagatgtaatcaggcatccaggaagctatctatatggaggcaaaggtccttttattcaggccactggaagcctcttccataaagttcagtagtacgagtacagtgtcctttcctgtgtacagcccctcgctttctcttctggactcccagctgagccagtgtttgagccacccatcactctgaaaacagcatcttcatctccttaggctcagcttctcaagtcacacaggctacattgctgccctcagggtgagcctcccttcattcatctcggtgataattctaaacaatggcctgtgtgttatagaaaggccctgcaagcatacatgttatcaacttactagctgtgcccaaggttgcatagctagtaagtggtaagactgaaatttgagcctaggggaccataactctaaacaatgttctatccactaggcggtactgtgtagaccatgggctcacacacacacacacacacacacacacaaaatgtattgaataaaataattgtgggttttgcatattttcctgttttatgtcagcttgacacaagctagaatcatttgtgaagagggactctcaattgagaaaatgcttccactttttgttgttttgtttgttgtttttgcctgtcggaaagtctgcact

SEQ ID NO: 24 - DNA, 490 bp, Homo sapiens
ctgtggagtgcctatagcactgtgtgtaggcagaatgcaaaggggacagtgtgggtggggacagtgttggtgtagaaatggcggggaggttagattgcaggcacagagggcctcagccatctcgagagcccagacttcctccctgaggtgatggcacttggggaagtcagtcatggaaggattttaagaaagatgtgaaaggggcaggtttctattttcagaaaaccattctgggccagtggaagatggagtacacaggaccacaccttggtgaagggagattgtaggagcctgggcttggtggcgggggacagtggagagaacagcctgggatgtatgaacatggcaagtctcccttcctggacagtggggtttgcctatggtggacagaaggtgagatcatcctttgaaaaatgccacttcatagtgtttccccagctgtgggccttcactcattggagggtcaaataatcaatgtattaggttgcaa

SEQ ID NO: 25 - DNA, 1505 bp, Mus musculus
tcccagagaacctaagcctgattcccagcacccaaaggactgcttacaaccaactgaaactccagttcagggatccaacaccctcttctggcctctgtaggcaccaggcttgcatgtggtacccagacattcgtgcaagcaaaacactcatacatataaaaatagataaataaatgcctatttaaaacccttgcctcatctgaaattatctgaatgttgatttctttggattccctttccttttgcccttgggaaaaataggtcacccctgtgtcagttactgtatgttttggtcactgttcatagttttagagaggatgtctaggagggcagggtcacctgtggtgtggcaattgggagctccatgtgcagaaggaatgcagacacagcagcagagagtgcaggaggcccggaaggttccaccatccccacagccccacttcctccctctgccgaaggggttgggggtcaggcagaggctttaagaggggcgtggacagggtagatttctgttttgggaaaaccatctatcagagggcagaggacagggtggaacccaacacagctgagagcttgcaaggggctgggctgggcagcagtgaagaggaacctcacagggaggagcccctggggtgcaggggctctgaaactgccctgtgaaaaacactgcctcattgtcttggcagtttgggccctgacccagtagcagcaggtcagacaattgttatataaagttccgaaaattcaaacctcccccttcctccttcatccttcttagctacacgtgtgtccatgagtggcagagcaggcactcacatagaggtgtgcccactgcagcggctacagcactaaagaaaatccctctctccccttcctctccccctttcttttacttcaaagcagagtcttactatagggcccggcccctgtgggctgctcacttttaatcctctgccttggcctatctagcactgagatcacacacctgcctgtgtcactatgcctggcttccagcacttctttgagtgctgacagacacctcaagtggaaaattcttgtccttgcttcatttgacagatcacagtgaaaatgggagcccactaaaaatactttataggattaccctcgggctgtgtctgaggcgggtaggtaacataaggaatttcagggttagactttagtcctgtcaccaagacatctatctctttatacatataaaagtattccacagtctgaaaaaagctctgaaatagagaatgcttcttgtccatagcatcatagatagagacccttcagacttgtatataaaacagaattgaaaagtcaattcaggtgtgcacacacacatgcatgcacgcaccagcacgcctgacatctctcagggctgccgggcatcactcaggtgactgcttgacgtgttgatgtttgtgtctttggcttcttctttgagtcttttgtttttcttcttttattttatttatgagacagggttgagttcattgcat

SEQ ID NO: 26 - DNA, 1840 bp, Homo sapiens
cacaccattgcatgcttcagccgttgcccgtgctatttcctcccttggaaagccctctactgtgaggccctcacctctcaaccctctccctggcccccatgttgtctatgtgatttcttgccatttaaaaatctacccaggtgtcagcgcttgggcagtttcctcacacctctcacccagttcatcctcccttgcttggtgctatttctgcccttgtccatatccccaccacagcatgcactttggattccaggcacgctccttgagtgtgaccccgaggccctctgtgggctcttggagcagggcaaagctgggtgtgctggggcgcagcacgggcctgatgccctgaggttgtttgttgtgctgggctggaggcgttcgaagaaacgtccaaggaggctgctagactcagttctttctttctgttttccctccacctcctctgctagtggaagctccatgtctcccaggctcgtgagctggcaaacaccccgcttgcatggttcagtgttgtcgttggcggcaggcgtacgtggaaggccagttacagagggtctctagggctaatgcatttcacaacacaccgccctctgacactccacgctctgcttttcctccagaaccactccctttgcaaaactctgtttcaaacaaaaagagcacaaagaggctgaccgtgccttcctccaaccaagctcccctctccacaggtgcacagcaagagccctttgtctgtgatgggacaggcctgggctccagtgagcaagacaggcactgtgggcccatccaaatattaactgtggacactttcctactttgaaaacatgagactttgtactcagagccctgccctccagagaacacaattacttctgtttttcttttcctagtggaaggaggcttgacactggtgatggccttgcctttacaatgctcagggtttgggaaagtcagggcctagggctgctgatctccaggcactgtctgctttccatctatcctctctgcttggtccctgaaaagcaggagggagacaggaggaatgggagcatgaatgccctcagggtccacgggggatcccggaaggcctagaacaccaggggtctgggctccacccatgatggatcatgcctttgggggaagattggcctacactcatgtcaagtaataagttttacttcctgcacctggtgttaggttggttctaagatgcagctgtaacctgtgactaagatcaatatttttcatgtcactatctgatcatacaatggtcaatttatcgatttagaaaattgttgcacaacgaggcaacaccgagtcatgacttaaaaaaaaaaaaagtggatctaaccgaagctagattgtggcttatcacctttgattgtcagtttcttgggtcaaatcttaatgccacattgaccactgtgtcaagagaggccaggttccaactcagctccgtgtatagtgttcatggaatctcaatgctcatcaggcgctgctggggctgggcctcggggaggggcaggctcctgtcagcacaagtcaccagcacaggttttaaccagccagtctgggctacttttaccactgaagcagtggggcgagaaactctattttacagtgtttctaaaacctctgtgagctaaaagtagaagcaactcaaatgcccctcacctgatgaataaacaaacacagtgtggcatcctcgtacaatggagtattattcagccatagaaagggaggaaatagttgtgctcgatacagtatggatgaggcttggagacatgatgataagtgaaaagaagccaatcacaaaaggacaaataatgtatgattccat

SEQ ID NO: 27 - DNA, 1451 bp, Mus musculus
taagccatcacatgcttcaaccatgggctacttccacctgctccccccccccccacacacacacactgctacccctcacccccagcttggtgcctcacttctcaggctataatgctgctttcatggacattccttgttctttggaaacaagggcccttccctctgcagagttctcctgcctgaggctgtgtgttcttggtttgtgggcctttgcccagctggtgcccagtgcaaggtgccctgctaactgaacaaatgaccttgctcatcgtcatcttcttggtctccatctttgtggtggagccttctggaccaccggcaggtaccctttgcaggacagcctatcctgccctgtctccctacagagccactccctgaagctgcagaaaacaagagagcatagaggtgaccctctccacaggtgtgtggccagagccactcatccacagtggccaggcccatccaaatattaatgatgggtgttttctgctttgaagttgagaatgtcggtcctcaagagtccaccctgaagagaacacaaccacatctgtttccttccagggaacaggggctgcactgcccttcttctctgtccgtgcccagagcatgtatctgagcatgcccagagccaaacacagcatctatttcctactgatcttcacagctggacaggctcccacacagccagatgctccctggggagcctcaaaagcaaggttcaccaggtggagctctggggaaattgctttcaactctgtcttggcagggcttgccttctgcacctggctttaggagggctccaagatgcagcataacatgggacggatatcaacgcttctgtctgatcttataacaaaggtcaatttgtaaagttgataccaccaagtcctttcttccttcctttcttccacaccccgtcctctctgagaaaatggatccaatagaagctagagtgtgacttgtaggttctgactgtcacttctttggggtgaattttaatgccaaatcagccaggggcgaagctgaggagagccaagttcacacacagttcagcacgaagttttaattcagtcccatccgtccgaatctgcactgctgtgggtgggttaaagggagagcaggctcctgacagcatgtgctccagcacaggtgagtctgtcacactttttcctacagctgccaggcaagacgtcaagtctacttaaggtttcttatgcctggaatcgcctaaaacgtaaagcaatcaaaatgtctatcacccaaagagtagccagacaaaacacagcaggtccttttatgaagagtcctgtgtcacaagacacaggaatatcaattctcagccattaaaaggcacgctgtaatgacactggccacgatatgccacatcttagaaatattacaataagtcaaagaagccagcagcaaaaggctaactaatgtattatttccat

SEQ ID NO: 28 - DNA, 6212 bp, Homo sapiens
ctctaggtggtgaaaatgaccagatttggttgtggggtcatagtggacactaaagatcagcaagggaaaaaagatgtgactataaactttccattctcacagttgttttgagacccgagtgtacgtttaatgttttcaacagaagaggctgcatgaagaagagtaagttaaccgcggggaggctgtgagaatttttctgcgcggacaatggagctcagtgtctgtttcagtgtttgtgctctctatagatacctggatgattcttgggcctcagtgtgttctcgctccctccctgccgagactcaaagggatgatgcacgctgcccagccaaaaccaggacagaacgtctttttccccgtgggaatgcgctcccggcgccaattccaaggcctgcctgggtcctattcaggcagtgctggggtgagcagcaggctcgggcccagctgacacggccagagatccccagtgactactttcctgacatggcagagatggcagatggagaatccataagccccagttacacccgggagctcacactgtggcttcagtctccaaggagagtggggagagccctggccctccgtgaaggattgcttccgcccaaggggggccagtgaacccgaatcactctgctggatggtgctggggggctgatgcaatctgcattccttcccctcgcaccccttacccctcgctacctcccccttctcatcctccccactcgcacctctccttctcccacacctggctgacacccactcttgagtcactgtcagctccaagacagaaccggcatcctgggtgcttggcaggagccaaaggagcatgttacaggatctctggcttcacagatggggagagagcagttcagagaattgcgggttccacatttgcttgaagtcactcatcagcctttatgttacattacaacaaagcagcccaggggacatggactcatagggtacctggtgtttccccaactgtaggggggattccgggacaaataaagtttgccactgggaccctcccccgaactgtgccctgtcccactcctgtgacacactctctgcccacaagagagtggccaacagtggaggctgagagtgaccacctgcctgccctcagttattaaaggctactggagaacaagccttgagtgcgtgctgagaacacatgcccctagctgccatcaaagagaatcacttcatatgattttgaccataagcaaactcttccaccttcattttttaaaataacggctttattgagatatgcatcacttaccatgaaactcactcttttaaagtgtacaacccagggttttcagtgtattcacggaattgtgcaaccatcacccatcacccctaatttcaggacatttttatcactccaaaaagaaactttgcacacatcattcttctctccccacagcctctgacaactgctgatctattttgtctctatggatttagcagtcatggacatttcatatacatggaatcatacactatatgtcctttcatgactgacatctgtcacttagcatgattttatgagattcatcatgttggagcatgcacccatgcttccatcctttcttttttttttttcacagtcttgctctgtcgtgcaggctgaagtgcaatggcacgattttggctcactgcaacctctgcctcccaggttcaagccattctcctgcctcagcctcccaggtagctgggactacaggtatgtgccactatgcctggctaatttttttgtatttttagtagagatggagtttcaccatgctggccaggctggtctcaaactcctgacctcaagtgatctgcccgcttcggcctcccaaagtgctgggattacagacgtgagccaccacatcctttctaaggctgaatagtattgcactgtatggatagaccacatttagtttatctgcctgctggcttatggacaatgagtcactccactttttggctactatgaatcatgctgttgtgagcacttgtgtacatgtctttatatggatgtctgttttcccttccattgggtttgcttgggggtggaattgctgggccaccttctttctccatgagtggagcatgcctatgcgcccatccccgcatctcccatgtgtggaggcactgcccaagctcgtctgtactctgagtcacagggctgtgcaccattaccgatcaccatctatgggtcagggacttatcaatgagcaagacatagcccctgccatcactaactcacattctgcatcgtcctgtgccatccccaccaccccaccttggtcaggcccagtgtccaggtgtcttcaactgctcaccttccccctattttgttgccctgaagttcatccagacatcagggtgccctattgaaaatgctagttaatatgacctctctgctctaaccccaatgttggagtcttgtcatcagtgggatagagctggtgtgactgcaccagaccagtcaggttcaacttttatgaaaggaagttgtgagttgctttcagttgccatggaccccaagtcgtaggtcatgtaagctgagcatgcccaaacggaccaagcatgcaaccatgggcagaacctgagtgctcagactgaggagcaggggctgaattaagaagcagagcatacatggcaggatccaggatccaggagccaatcagactgagtttggcatcactccatggcaggatccaatcagatcacacctccctgcagcacctcattgcaagatccaatcagaccacacctcattaccctaggcttataaaatccaggccagccgctagcttggggaggcagatttgagtgtttttttttttctgtctccttgccagactaccagcaaaaaaggttttcttttctcaaaagccggtgtcatggtattggcctctgtgcacattgggcagtgagcccactgattgctcagtaacatgggcacactctggggcccacacaagccaggaatgatgtggcctttacctgctgctccagctgcatctgagcccagtatcccctgaacacaaacccccacctgcatggagctgcatgcggttctcgggtacctcctggctatgttcagctcctgtagattccttcagatccactccttcccatttcctcatccaactgcccagcagagtgcctactatgcgccacacactgggattcagcagtaaacgacacaaacatgatccccacccttatccttctcccaggactcttattaatctaaggctcacctcccttcttgtaacttccatgaactcatatgctccctctcagctcagggacgttgctggaggaagcaagagagcagcagatgaacccttatgttcaggaggcagatggagctcattcaaagcccaccttggcctcttcttaacccgaagattttagcaagtcatataacctttgaactgcaactccctggattgtggaatgcccaaagtgtgctgagcgtgaagtaaataatgcaagtgtaaagtgtgcggcatggtcctggttcatctcaggaggccgttaggaaactagcacttatttttgccagggcttgagcatagaacatactaatttccccaatggcattatcacattgtattactttttatttacatgttctttctcccctaccaatctcagagaatctcaagggcagcaatgattaattattaattttggaatccttggttcctggcacattccttgaaaataaatcattggcttactttccactgattctcttaattacccctgagaggcagagattggaattatactatgctgagcagctcaatgttttcccagtaacagcaggaaaatcccaatgcacagagaaggaacctgaatgacttaggtgggacacaccaggacagacacccgtggtgatgacattctgtgcccttcatcccacagagtggtctgtcttcacagtggtctcccctcaccacactgagccctcaaacttcctctttccgctgaccaaagtgcacccaggcctgcttgtccattcagacagatgccagggccctctgcactccatctgacctctgcaatatgccggttcctaataagggagcaggatccaggtccagttgttcacacttctaatttcataccggcagcctcagtaaagttctgccatcaggctaaggccccactgatcgtcgaccttttctgcataaagattcacctccagggctcttagaaaatactgctgcctggctaccaccccatccttagtgtgacatagggtttttttttcttcttcttctgttttttgttttttttagaataattaggcagctctgttgcccaggctggagtgcagtggcatgatctcagctcactgcaacctctgcctcctggttcaagcaattctcctacctcagcctcttgagtacctaggactataggcacacgccaccatgcccggctaattttttgtatttttagtagagacggggtttcaccaggttagccaggatggtctcaatctcctgaccttgtgatccgcccacctcagcctcccaaagtgctgggattacagacgtgaggcaccacacctggcctgccccgggttgtttttttttttaaagctccccagggatttgtaagtgcataccaaagactgggaacccctggcttagctcacagagcaaagagccttttgagggttcccctcgacagttgctccctcacctccagctgtggggccacacagagcgctgggccattgtggtgttagagaccagagttaaagggactccatctgtaatatccaggacaaatgggctggcaggtgctgctcaaacccttacacacagatagtatttggggaggtgaggtcaattcccccattatggaacgctgcggttttaaaagcaagcaaacaaacaaaaacaggaaaaaagtgagctttttaaaactaaggtaaaatttgtcctcaacttcctggccttgattgggctctgctactagagcggcagaagcaactcacttccctgcttccacggacctgtttcatgtaatgcattttgcagagatttgaagacagggtccttgacttgggcagctaacagcctgaggctagaggcagccacccctgaacagtgaacaattctgcaaggcgcctggcaatagtactatgcggggagggggtaggaacaaggtgctgcagggcggggtggaggaggaaatgaattctgcctgggagaagcgggagtgcgtatttgagtggggtctggagcaggtgcatgcaaagaagcacctcaaaggcacgggcaggtgtgtgcaggcgtgggcaggcgtgggcaggcgtgggaaggcgtgggcaggcgtgggcaggtgtgggcaggcgtgggcaggcgtgggcaggcgtgggcaggtgtgggcaggtgtgggcaggcatgtgggcacggcacagggcttgtccaggccagatgccattaagcacaggtatctgtggtgggcaggggacacagtggaagcagatagagaaggtttgctggggtcccatggaggggcgccttgtaggccatggtcactctaggctgatgcaaggtgctcaaggttgaaggcagaggtgactgacctgtgcttgagagagggtagggaagagaagctgccggacttgaggggctgaaattgtcctgtaatagtccaggtcaggagtgttaatgatgccccagctcgggcagtgactacggcaaggagagtttaacatgtggttcagttcagcagacatggggaactcactatgtgtgaagcaggacacatcacggaggcagccctcaaatgcttgaagacagtaatcctgcccctgtgctgtggcgggttctttaaggggtgtgacttcctcatcagacccattgctctcacacctaatgatgctgccatgtggcagggctgtgggcagagccatgccctagcaggggaagtggaggacagcggcggggagggagtgtgggcagggctttcctgccctctgggtcctctcctctctttcgtggcagggccttgaggtccattcgctgggctgcacagaaggaggactccagagccccccttgggttcaggattttatacacgcagcattccagacagatggacccgtgtattgacaatgaaagcatgggagaactgtatttctttggtgattaaagtaaatgcaaaagttatgatgc

SEQ ID NO: 29 - DNA, 2501 bp, Mus musculus
cctcagctggaattaaccctacacagttcctcagagcctagggcttagtaaaaaggccaagcctgacctatgacctctctgacatctgtccttagcacgtgttcttttctttccaagtacattgtaccaccatgatggcctgtgccctcctccccatcacctccatacaacgaatgagctctcatgagagcagagtggaggctggtgctgtggcctccactcaggaattgtgaaccactccaaccttcttttgttaaacattacctagcctcaaatatcttgtgatagcaacagaagagactaagatacttaaaaatatctatggatgaagaaaatgaccaatgtgaggacgtcgtggatattggccatcagcaaagaagagagcataaagttcccattctcacagatattctgaaacctgtgtatttcatttttgatggaaaagagctgcacacagaatagtaagttagctggagggaacttatgagcctttttttttccccctcacataaacaacaatggagcttagtgtccatttcattctctttgtgcttgactgggacccagatggctcactgtccctcagtatgtccctgctccctccctgctgagatctcattggctgtgacgcactgccctgctccagccaggacactactgtctttcttccccgtgggaatgtgttctcaaagccaactccaacaacgctgacctgggcatcacttgggtggtgctggagtgagctgtaggctctggtcctgctgttgtagcctggggtcctagttgtcattcccctgacacagcagagagagcaaacaacagaaccaatggctgtagccacatggtgaacagctagacctccagaacaataggagtaaatgcttctgccacgaagtgtatggagaacctaaaccaatcttcaggcagaactggggccaggtaccacacacagccctgcccctttctcagctggctgttgcccatgccagagtcatgatcacccataggattctcagacccagggcattgtgtagctggagctcaatgagtcttacgggccggaagcagccaattcagggaactctgggttctgcgtttgctttgcatctatttggtgagagacagtgtgagttcttccattacaaaattccaatgtttaaagagcaaacagtcaagaaacaagaaaaaaaaacccaagggtgtgtctgtgtgtgtgtgtgtgtgcatgtgtttatgtatgtgcaggtacatgttggggacatgtgcatgtgcatgtttacatgtgcatagagaggtcagaagacaacaccagctgttgttccccaagtacaatccatagttcaaccccctgtgtgtgtgtgtgtgtgtgtttatgtgtgcatatgctatggaagtcaaagattgagtctggtgtcttcaactgccctctaccctattttctgaaacagagtctctcactaaatctagacctcactggttgggcatccttgttagccaatgagctcaactatctgcccgtttgttctctctctctctctctctctctctctctctctctctctctctctctctctctctctctctccataaatgaatgaatgtgtgtttttaaaaagagagtttaaaaaaaactaaggtggcatgtatcccagcttctctccacaatccaactggaacggctcaggccagcctcatttcacgcagctcactctatcaacacatctgctgcacagagcatgctttgtgagtgactcaaagatcagaaccctgacttccaatggcttatagcctaagggtagagaagttacctgtattctggcaagataccagggattgtaggaggggtagcaacctggggaggagggaatgcactctgtgtaggagatgcagaaaggattggaagagctggtgagtatttgagttggatgttggactgataaatgcagggagcatctcacaggttgggatcaggcacaccggtaggatgtttcatccatccgagtcaaatggagggcaggtgtagggatttcaggttagagggcagggaaagaaagtagagaggagagcctggggttgtgctggagtgtgcacagagcactcagctggcactttgaagaacaaagtggactgtccctggacgtgagactgagcaggtaaggtgggttaagagacggtaagatcactactgcaataatccaaaataagaacctttatgatctctaggtgggataacaaccagggggagggacttttaacacacaattcagttcaacaggaactcgcacatcctggaggcaacacgtgaactgcgcaggctcagcagtcattgtctgttctgcgtggtgctcttccaagtggcacagtgtcttcatcagacctggtgctcacatgactgatctagtcacagaacaggccatgtatcaagttttgggaaacaggaagcaatgggagaaatgtattttattggtgattaagtgaagtgcaaaagataggacgtgcta

SEQ ID NO: 30 - DNA, 347 bp, Homo sapiens
gggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtag

SEQ ID NO: 31 - DNA, 1131 bp, Artificial Sequence
  Synthetic
gggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagcaggtgagtttgtggtgtcgccgatgtcccttcggggtactctagcgcagccgcctggctacttgacccactgccaccaaacgttttaaattcaccgaaagcttagcttcgaagcaaagctccgtttcgccggtgaagcaggaagccttcgctgcaggaactgacctttacctcttggagcggcttctgcagaaaaatccccgggcagagatttgggcggagtttgcctagaactaacgcggagccagccgatcccggcctaccccggggccaagattttaaggggtgaagagtcccttttgccttttctggatcctggtgattcacctagtgtcttccctaaggaactgaaccaactcctccgctggcctctggcagccctccaggcggtgcaggatggcgtgggcccggtaggaagctgcatgtaaccgcccagggtcgggaggccaggagggcagctcctcctctgacttgaatattgaaaacaagaggatgcttttaagaaaaagaagaaggaggattcactaccagctctgaagggtggaaaagagatgattcatccggattgtggagagggtggaatcttgtttaggagagcgttggttgtggcaggcagggtgtaactatgaatcagtgaagacaattcacatcctgggatgaaaagaaggccatgggctcacaggagattatccactggcctctccacatccgcttgcagtaaggagtgtgggactctcccaagcttcagcgctgaactgcaatgcagtgacgtcgcttaaga

SEQ ID NO: 32 - DNA, 1431 bp, Artificial Sequence
  Synthetic
cgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataa

SEQ ID NO: 33 - protein, 743 aa, Artificial Sequence
  Synthetic
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQSTTLYSPAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 34 - DNA, 149 bp, Artificial Sequence
  Synthetic
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagat

SEQ ID NO: 35 - DNA, 139 bp, Artificial Sequence
  Synthetic
cccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggcca

SEQ ID NO: 36 - DNA, 6374 bp, Artificial Sequence
  Synthetic
ttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagcggatccgccaccatggattggggcacactccagagcatcctcgggggtgtcaacaaacactccaccagcattggaaagatctggctcacggtcctcttcatcttccgcatcatgatcctcgtggtggctgcaaaggaggtgtggggagatgagcaagccgattttgtctgcaacacgctccagcctggctgcaagaatgtatgctacgaccaccacttccccatctctcacatccggctctgggctctgcagctgatcatggtgtccacgccagccctcctggtagctatgcatgtggcctaccggagacatgaaaagaaacggaagttcatgaagggagagataaagaacgagtttaaggacatcgaagagatcaaaacccagaaggtccgtatcgaagggtccctgtggtggacctacaccaccagcatcttcttccgggtcatctttgaagccgtcttcatgtacgtcttttacatcatgtacaatggcttcttcatgcaacgtctggtgaaatgcaacgcttggccctgccccaatacagtggactgcttcatttccaggcccacagaaaagactgtcttcaccgtgtttatgatttctgtgtctggaatttgcattctgctaaatatcacagagctgtgctatttgttcgttaggtattgctcaggaaagtccaaaagaccagtctacccatacgatgttccagattacgcttaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcctt

SEQ ID NO: 37 - DNA, 700 bp, Homo sapiens
ccatgatatgttaagaaaagcaaagtgtggaatagtaggtaaaatattctatcttatgtgcaaaaggggaaataaaagtcatcaatattcatgtagattcaattcacatatagattcatatcacattcctatatatatagaaattctggaaagacacaaaataaattaataaaagttgttacttcattgtagtttttaaagttttttgagtcttaagacttactttccacttctgtagaaaggaattacaaatcctttctttatagagctatgtgatgaaataaacataaagcatttggcacacttcaggatagcaacttgtggattaatgattaacacagtcacctttgcaccagattacacccagagattccttcatttatatttatgtggttttgtgtgtcagttatgcagtctaactcagtcattcaactatgttacagctgcaacactctatttttttctttggtacaggagtcgccctcttatccactgtttcatttttgtggttccagttacctgtagtcaaccacagttggaaaatatgatagcattttgagagagagactgcatccaaaaacttatattacaatatattgttatacattgttataagtgttgttttattattctttattgttaatctcttaccattaagccttatggtaggtttgtatgtataggaaaaaacagattatat

SEQ ID NO: 38 - DNA, 700 bp, Homo sapiens
atataatctgttttttcctatacatacaaacctaccataaggcttaatggtaagagattaacaataaagaataataaaacaacacttataacaatgtataacaatatattgtaatataagtttttggatgcagtctctctctcaaaatgctatcatattttccaactgtggttgactacaggtaactggaaccacaaaaatgaaacagtggataagagggcgactcctgtaccaaagaaaaaaatagagtgttgcagctgtaacatagttgaatgactgagttagactgcataactgacacacaaaaccacataaatataaatgaaggaatctctgggtgtaatctggtgcaaaggtgactgtgttaatcattaatccacaagttgctatcctgaagtgtgccaaatgctttatgtttatttcatcacatagctctataaagaaaggatttgtaattcctttctacagaagtggaaagtaagtcttaagactcaaaaaactttaaaaactacaatgaagtaacaacttttattaatttattttgtgtctttccagaatttctatatatataggaatgtgatatgaatctatatgtgaattgaatctacatgaatattgatgacttttatttccccttttgcacataagatagaatattttacctactattccacactttgcttttcttaacatatcatgg

SEQ ID NO: 39 - DNA, 700 bp, Homo sapiens
gcagagacctacagacagaagtacattttacactggatccaggacacacatcagtctgaaaacacacacatgaaccaaacgtttcctaaagcattacttatccttgctaatagcaacacattctcatattcttttatacttcatttaatttcatataaaaaagaaaaggaaaggaaagaaatctatttctcagcccattaataaggtcaggagcagcaacaccagactagaagaaaagcttacctatagatttttctgccacctcttgagtgcgtccagctttccgacaagtctcagtgccatctactgtgcgctctgggtattgcaattgctttttttttttttttttttttttttttagaatgagactaagtcagagaacacaaagaacttctttccccacagtggagatggctctgaaagcgtttaaggaatagcttagatgagtggctaacacattctcccggttctgaattctaagaccacagactccatgtccagtccccaaagagaggctttgcaagctacagaatacccctctgactgggacctcaggagctaaactgaccacgtaattggttctagaaagtgaaacgttttaatttgaaacatccaaatgagcattttgtgaaaagctactgccgtccatcaaatacaacacagccagggagtcatcgctctattgcccttgtcaatccta

SEQ ID NO: 40 - DNA, 700 bp, Homo sapiens
taggattgacaagggcaatagagcgatgactccctggctgtgttgtatttgatggacggcagtagcttttcacaaaatgctcatttggatgtttcaaattaaaacgtttcactttctagaaccaattacgtggtcagtttagctcctgaggtcccagtcagaggggtattctgtagcttgcaaagcctctctttggggactggacatggagtctgtggtcttagaattcagaaccgggagaatgtgttagccactcatctaagctattccttaaacgctttcagagccatctccactgtggggaaagaagttctttgtgttctctgacttagtctcattctaaaaaaaaaaaaaaaaaaaaaaaaaaagcaattgcaatacccagagcgcacagtagatggcactgagacttgtcggaaagctggacgcactcaagaggtggcagaaaaatctataggtaagcttttcttctagtctggtgttgctgctcctgaccttattaatgggctgagaaatagatttctttcctttccttttcttttttatatgaaattaaatgaagtataaaagaatatgagaatgtgttgctattagcaaggataagtaatgctttaggaaacgtttggttcatgtgtgtgttttcagactgatgtgtgtcctggatccagtgtaaaatgtacttctgtctgtaggtctctgc

SEQ ID NO: 41 - DNA, 700 bp, Homo sapiens
atccattatttgattagccatttcaaaaacacatttacggagatcttcatctgggcagagcattattccaggcctctgaagaaccaaagatgattttgaaaggaggtcacagtgcagacagcaggtgtgtatataaggtggctactttacaaaacaggatatggcaagctggacatgacaggcacagcaaagtctctgaacagagttcggggcatgaaattgtttcttttgggggtcttcaggaacaatttcatgaaagctaaatcatgaaagatagcaggcttttgccaggaaaaaaaaaaacaagactagtgattagtttggcgttttcggtttctttgagaagcgaaataacttatcaaggactctttttgccacttgatgttataattggttgataggtctctcagaagccctttgtgcaaactagaacctgcagggatgtgcaaagcctctctctgctgccatctgctgtcttacaagaggtaactgcaagaggttgaatcctccaatgccctggggattcccattgcagggcaggggcagcagcctgtgttaataaccacccgaacagccacatgtacccctccacaaaagtgtcactgtctccattgctctggagtttgtattcccaatttgtaatctttgttagggcactcataaaaaattaaaaacaaaaattcacacaaacatacactac

SEQ ID NO: 42 - DNA, 700 bp, Homo sapiens
gtagtgtatgtttgtgtgaatttttgtttttaattttttatgagtgccctaacaaagattacaaattgggaatacaaactccagagcaatggagacagtgacacttttgtggaggggtacatgtggctgttcgggtggttattaacacaggctgctgcccctgccctgcaatgggaatccccagggcattggaggattcaacctcttgcagttacctcttgtaagacagcagatggcagcagagagaggctttgcacatccctgcaggttctagtttgcacaaagggcttctgagagacctatcaaccaattataacatcaagtggcaaaaagagtccttgataagttatttcgcttctcaaagaaaccgaaaacgccaaactaatcactagtcttgtttttttttttcctggcaaaagcctgctatctttcatgatttagctttcatgaaattgttcctgaagacccccaaaagaaacaatttcatgccccgaactctgttcagagactttgctgtgcctgtcatgtccagcttgccatatcctgttttgtaaagtagccaccttatatacacacctgctgtctgcactgtgacctcctttcaaaatcatctttggttcttcagaggcctggaataatgctctgcccagatgaagatctccgtaaatgtgtttttgaaatggctaatcaaataatggat

SEQ ID NO: 43 - DNA, 700 bp, Homo sapiens
gctaattgggtcaggatttgaaagaccttagctttgtgtgaccttcaattttatcattcagcttgaatatgtgccccagaaaacctttatgtaattccctaatatttcagtaaccagcatgcaacatacgagaagcacattctttgtttttagaatggtatctggctgatgactttcacaacagctcacatgagagggaagtattttagcaatcggactgaaggaaaatccaaaaactccaccattgcagggtcaacagtgcacgtgtttgaattctgaaagacgtaagccaaggcaaatagaaggaaatgatcttccactaatcccggcatttacttcctcctctctggaggggacggccatgcacacagagccctgtgctctgagttctcatgaaagggacacagctgggctcactcagcgtcacctcgcccctggggtgtgtcctggtttcagatctcgggctggagtgattcacgtgtggcagggaggccatcattaatgaaaatgcgagggcgtcgcacgagtgttgatgactcagcaggcctttctacttctgtatgagtcagtgcccatcacagccaagcctggggcacaacaggttttcttaaaagagcatgggggcctcatcttcaacaaccaattaggaagcagaaaagtcctcagtgaggaaggaataatgacatgttggagctaagat

SEQ ID NO: 44 - DNA, 700 bp, Homo sapiens
atcttagctccaacatgtcattattccttcctcactgaggacttttctgcttcctaattggttgttgaagatgaggcccccatgctcttttaagaaaacctgttgtgccccaggcttggctgtgatgggcactgactcatacagaagtagaaaggcctgctgagtcatcaacactcgtgcgacgccctcgcattttcattaatgatggcctccctgccacacgtgaatcactccagcccgagatctgaaaccaggacacaccccaggggcgaggtgacgctgagtgagcccagctgtgtccctttcatgagaactcagagcacagggctctgtgtgcatggccgtcccctccagagaggaggaagtaaatgccgggattagtggaagatcatttccttctatttgccttggcttacgtctttcagaattcaaacacgtgcactgttgaccctgcaatggtggagtttttggattttccttcagtccgattgctaaaatacttccctctcatgtgagctgttgtgaaagtcatcagccagataccattctaaaaacaaagaatgtgcttctcgtatgttgcatgctggttactgaaatattagggaattacataaaggttttctggggcacatattcaagctgaatgataaaattgaaggtcacacaaagctaaggtctttcaaatcctgacccaattagc

SEQ ID NO: 45 - DNA, 658 bp, Homo sapiens
cgcctcggcctcccaaagtgctgggattacaggcgtgagccaccaccgtgcctggcttatacaagtaattgtaaacgaaaaggaaaaaatggagatacagttttctcgtgcatcttaaactttggtgcttaaaagcaccattaaattctgctttcacatgaacacacacaagattaccacgtttgctctgggctgctgcgtattggaaggacatacacattcaacaaatatttgttgaacttccattctgtacacaaagcacaaagaaagattcgttcacagtccgtgtgggtactggaaagcagttccagccctgcctgccagggggcaccccaggcaagcacatctcagtggctgctagaaagtgaattgaggctgagtctctccacacccaagtgttaggcgttctaggctcagaaagagacaatgacaatgcgggcaattctctcttcactgtgtcctcttctttgctagaaatgttattagaatatggaaatgtgacattcagcactaatcagtttgacatatgaatatatctatacacatatttctccctgaaattggcctaaatactctttcttggaaccaaatgagaagcaaacaacctttacaactaaacattaaaccataagatgaacatcttagttgtctacctaga

SEQ ID NO: 46 - DNA, 682 bp, Homo sapiens
ttctaggtagacaactaagatgttcatcttatggtttaatgtttagttgtaaaggttgtttgcttctcatttggttccaagaaagagtatttaggccaatttcagggagaaatatgtgtatagatatattcatatgtcaaactgattagtgctgaatgtcacatttccatattctaataacatttctagcaaagaagaggacacagtgaagagagaattgcccgcattgtcattgtctctttctgagcctagaacgcctaacacttgggtgtggagagactcagcctcaattcactttctagcagccactgagatgtgcttgcctggggtgccccctggcaggcagggctggaactgctttccagtacccacacggactgtgaacgaatctttctttgtgctttgtgtacagaatggaagttcaacaaatatttgttgaatgtgtatgtccttccaatacgcagcagcccagagcaaacgtggtaatcttgtgtgtgttcatgtgaaagcagaatttaatggtgcttttaagcaccaaagtttaagatgcacgagaaaactgtatctccattttttccttttcgtttacaattacttgtataagccaggcacggtggtggctcacgcctgtaatcccagcactttgggaggccgaggcgggcggatcacatgaggtcgggag

SEQ ID NO: 47 - DNA, 135 bp, Homo sapiens
gggttcggggctttcgggggcggtcgggggttcgcggacccgggaagctctgaggacccagaggccgggcgcgctccgcccgcggcgccgccccctccgtaactttcccagtctccgagggaagaggcggggtgt

SEQ ID NO: 48 - DNA, 7163 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcagtgatgcctgaaacctcagatggtactgaaccctctatataatctgttttttcctatacatacaaacctaccataaggcttaatggtaagagattaacaataaagaataataaaacaacacttataacaatgtataacaatatattgtaatataagtttttggatgcagtctctctctcaaaatgctatcatattttccaactgtggttgactacaggtaactggaaccacaaaaatgaaacagtggataagagggcgactcctgtaccaaagaaaaaaatagagtgttgcagctgtaacatagttgaatgactgagttagactgcataactgacacacaaaaccacataaatataaatgaaggaatctctgggtgtaatctggtgcaaaggtgactgtgttaatcattaatccacaagttgctatcctgaagtgtgccaaatgctttatgtttatttcatcacatagctctataaagaaaggatttgtaattcctttctacagaagtggaaagtaagtcttaagactcaaaaaactttaaaaactacaatgaagtaacaacttttattaatttattttgtgtctttccagaatttctatatatataggaatgtgatatgaatctatatgtgaattgaatctacatgaatattgatgacttttatttccccttttgcacataagatagaatattttacctactattccacactttgcttttcttaacatatcatgggatctttttatataagtgaacaaagagtttcttcattctttcacacagtttaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 49 - DNA, 7247 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgctatctatcatcttgaagggcttctggaacaagttagaatagagtcaacactcatgaactgctgtagcaaaaaaaactatagatgtaggattgacaagggcaatagagcgatgactccctggctgtgttgtatttgatggacggcagtagcttttcacaaaatgctcatttggatgtttcaaattaaaacgtttcactttctagaaccaattacgtggtcagtttagctcctgaggtcccagtcagaggggtattctgtagcttgcaaagcctctctttggggactggacatggagtctgtggtcttagaattcagaaccgggagaatgtgttagccactcatctaagctattccttaaacgctttcagagccatctccactgtggggaaagaagttctttgtgttctctgacttagtctcattctaaaaaaaaaaaaaaaaaaaaaaaaaaagcaattgcaatacccagagcgcacagtagatggcactgagacttgtcggaaagctggacgcactcaagaggtggcagaaaaatctataggtaagcttttcttctagtctggtgttgctgctcctgaccttattaatgggctgagaaatagatttctttcctttccttttcttttttatatgaaattaaatgaagtataaaagaatatgagaatgtgttgctattagcaaggataagtaatgctttaggaaacgtttggttcatgtgtgtgttttcagactgatgtgtgtcctggatccagtgtaaaatgtacttctgtctgtaggtctctgccacagaaaagttggaaagccattgttgtattccatttccagggcaacaaaagataccactgtcacttcatgtgaaatggtgttgtttaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 50 - DNA, 7243 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaagctactaactacaaccacgagattatagatgtttgctgatattgttctcagtttggttattgtgttgtttatgaatgaaagtagtgtatgtttgtgtgaatttttgtttttaattttttatgagtgccctaacaaagattacaaattgggaatacaaactccagagcaatggagacagtgacacttttgtggaggggtacatgtggctgttcgggtggttattaacacaggctgctgcccctgccctgcaatgggaatccccagggcattggaggattcaacctcttgcagttacctcttgtaagacagcagatggcagcagagagaggctttgcacatccctgcaggttctagtttgcacaaagggcttctgagagacctatcaaccaattataacatcaagtggcaaaaagagtccttgataagttatttcgcttctcaaagaaaccgaaaacgccaaactaatcactagtcttgtttttttttttcctggcaaaagcctgctatctttcatgatttagctttcatgaaattgttcctgaagacccccaaaagaaacaatttcatgccccgaactctgttcagagactttgctgtgcctgtcatgtccagcttgccatatcctgttttgtaaagtagccaccttatatacacacctgctgtctgcactgtgacctcctttcaaaatcatctttggttcttcagaggcctggaataatgctctgcccagatgaagatctccgtaaatgtgtttttgaaatggctaatcaaataatggatacccttaggtatttttgcagaaacacttggcagccttccataatatccctactatgaaatggaaacttgtgaatgagatgtggctttaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 51 - DNA, 7253 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtctcagctggagtgacgcacctcatccatgcgggcctggcgtctggaaggtggctgggtctctcgggcttgagcaccatcatcttagctccaacatgtcattattccttcctcactgaggacttttctgcttcctaattggttgttgaagatgaggcccccatgctcttttaagaaaacctgttgtgccccaggcttggctgtgatgggcactgactcatacagaagtagaaaggcctgctgagtcatcaacactcgtgcgacgccctcgcattttcattaatgatggcctccctgccacacgtgaatcactccagcccgagatctgaaaccaggacacaccccaggggcgaggtgacgctgagtgagcccagctgtgtccctttcatgagaactcagagcacagggctctgtgtgcatggccgtcccctccagagaggaggaagtaaatgccgggattagtggaagatcatttccttctatttgccttggcttacgtctttcagaattcaaacacgtgcactgttgaccctgcaatggtggagtttttggattttccttcagtccgattgctaaaatacttccctctcatgtgagctgttgtgaaagtcatcagccagataccattctaaaaacaaagaatgtgcttctcgtatgttgcatgctggttactgaaatattagggaattacataaaggttttctggggcacatattcaagctgaatgataaaattgaaggtcacacaaagctaaggtctttcaaatcctgacccaattagctctctgttagctctctgactttggacaagctgtctggtcctctgaagcatactttgttcgccctgggtaggggccctctgttttaacagcgtttggcattaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 52 - DNA, 7057 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttctaggtagacaactaagatgttcatcttatggtttaatgtttagttgtaaaggttgtttgcttctcatttggttccaagaaagagtatttaggccaatttcagggagaaatatgtgtatagatatattcatatgtcaaactgattagtgctgaatgtcacatttccatattctaataacatttctagcaaagaagaggacacagtgaagagagaattgcccgcattgtcattgtctctttctgagcctagaacgcctaacacttgggtgtggagagactcagcctcaattcactttctagcagccactgagatgtgcttgcctggggtgccccctggcaggcagggctggaactgctttccagtacccacacggactgtgaacgaatctttctttgtgctttgtgtacagaatggaagttcaacaaatatttgttgaatgtgtatgtccttccaatacgcagcagcccagagcaaacgtggtaatcttgtgtgtgttcatgtgaaagcagaatttaatggtgcttttaagcaccaaagtttaagatgcacgagaaaactgtatctccattttttccttttcgtttacaattacttgtataagccaggcacggtggtggctcacgcctgtaatcccagcactttgggaggccgaggcgggcggatcacatgaggtcgggagttaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 53 - DNA, 212 bp, Homo sapiens
ggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtag

SEQ ID NO: 54 - DNA, 784 bp, Homo sapiens
aagcaggtgagtttgtggtgtcgccgatgtcccttcggggtactctagcgcagccgcctggctacttgacccactgccaccaaacgttttaaattcaccgaaagcttagcttcgaagcaaagctccgtttcgccggtgaagcaggaagccttcgctgcaggaactgacctttacctcttggagcggcttctgcagaaaaatccccgggcagagatttgggcggagtttgcctagaactaacgcggagccagccgatcccggcctaccccggggccaagattttaaggggtgaagagtcccttttgccttttctggatcctggtgattcacctagtgtcttccctaaggaactgaaccaactcctccgctggcctctggcagccctccaggcggtgcaggatggcgtgggcccggtaggaagctgcatgtaaccgcccagggtcgggaggccaggagggcagctcctcctctgacttgaatattgaaaacaagaggatgcttttaagaaaaagaagaaggaggattcactaccagctctgaagggtggaaaagagatgattcatccggattgtggagagggtggaatcttgtttaggagagcgttggttgtggcaggcagggtgtaactatgaatcagtgaagacaattcacatcctgggatgaaaagaaggccatgggctcacaggagattatccactggcctctccacatccgcttgcagtaaggagtgtgggactctcccaagcttcagcgctgaactgcaatgcagtgacgtcgcttaaga

SEQ ID NO: 55 - DNA, 771 bp, Homo sapiens
tcatccatgtccctacaaaggacatgaactcatcattttttatggctgcataagtcgttctttcaaacaccctgcagtcagcttctcctcacgagaaaccacatgaaagccctcggggaaatgcctctcgggatctacttttctttgtgtgtatcctacttagcctatcggtttctgcttcctgtggggctacagccgtctcgtctttttctgctggctcctttgctctgttctccagtggctatcttctttctcctttctttcaaatgttctcccttatcttctctgatacagacagaaggtcaggagccacgcccattacactgacagaacccgatgtcctgatgcgctctgtgcctcccagatttggatgtggatgcgaggcgagctggccagagagcaatcatttcagcgagggtcgttattcccatcttctctcttaggacggaggtagggggacttctggccccaaatgttccttcttccagctgtggctgcctccatcccgcagagtgagcctttaatttggagatcctaatgccccagtgctgtgccaggcacagtacacgttctgcatggaggacggtttacgctccccttacagaagaggaaggacactcagaaggctgaactgttctgcctaaggtcaccgagttgctaaggcaagaagcagcctccaattcctgccttactgatttctgggatgtgaaaccaaaagggtgaggcggcaagccccggctgccctcgggggctcttcccaagtgctctctt

SEQ ID NO: 56 - DNA, 771 bp, Homo sapiens
aagagagcacttgggaagagcccccgagggcagccggggcttgccgcctcacccttttggtttcacatcccagaaatcagtaaggcaggaattggaggctgcttcttgccttagcaactcggtgaccttaggcagaacagttcagccttctgagtgtccttcctcttctgtaaggggagcgtaaaccgtcctccatgcagaacgtgtactgtgcctggcacagcactggggcattaggatctccaaattaaaggctcactctgcgggatggaggcagccacagctggaagaaggaacatttggggccagaagtccccctacctccgtcctaagagagaagatgggaataacgaccctcgctgaaatgattgctctctggccagctcgcctcgcatccacatccaaatctgggaggcacagagcgcatcaggacatcgggttctgtcagtgtaatgggcgtggctcctgaccttctgtctgtatcagagaagataagggagaacatttgaaagaaaggagaaagaagatagccactggagaacagagcaaaggagccagcagaaaaagacgagacggctgtagccccacaggaagcagaaaccgataggctaagtaggatacacacaaagaaaagtagatcccgagaggcatttccccgagggctttcatgtggtttctcgtgaggagaagctgactgcagggtgtttgaaagaacgacttatgcagccataaaaaatgatgagttcatgtcctttgtagggacatggatga

SEQ ID NO: 57 - DNA, 699 bp, Homo sapiens
cttgcttacccagactcagagaagtctccctgttctgtcctagctagtgattcctgtgttgtgtgcattcgtcttttccagagcaaaccgcccagagtagaagatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtc

SEQ ID NO: 58 - DNA, 699 bp, Homo sapiens
gacactgcaatcatgaacactgtgaagacagtcttctccgtgggccgggacacaaagcagtccacagtgttgggacaaggccaggcgttgcacttcaccagccgctgcatggagaagccgtcgtacatgacatagaagacgtacatgaaggcggcttcgaagatgacccggaagaagatgctgcttgtgtaggtccaccacagggagccttcgatgcggaccttctgggttttgatctcctcgatgtccttaaattcactctttatctcccccttgatgaacttcctcttcttctcatgtctccggtaggccacgtgcatggccactaggagcgctggcgtggacacgaagatcagctgcagggcccatagccggatgtgggagatggggaagtagtgatcgtagcacacgttcttgcagcctggctgcagggtgttgcagacaaagtcggcctgctcatctccccacacctcctttgcagccacaacgaggatcataatgcgaaaaatgaagaggacggtgagccagatctttccaatgctggtggagtgtttgttcacaccccccaggatcgtctgcagcgtgccccaatccatcttctactctgggcggtttgctctggaaaagacgaatgcacacaacacaggaatcactagctaggacagaacagggagacttctctgagtctgggtaagcaag

SEQ ID NO: 59 - DNA, 700 bp, Homo sapiens
gcctgacacagtctgagcctcctcaggcggcctcaggggttgggatagagtggagaattcaggcaagaatgccaaccctagctccaggcctgggacccacaggcctggggaaaagagtggttgccccgtcttgagacagccgaaaactgtgtccccaggattgttggtttcataaaagcaagtagctagggaggccacatttacaggggatcacagaacacttgggtaggggcttgctgtaggtgtcatcagggaagtgggggacggcaggagggatgtggcccagtacgcagatgaagacaggtgatcatccgctgggccacacgtggcagggatatgggcagagtgagcttggctggccccaggctccaaagctgcccagcccccgctgaaggtgaggcctcagctggtgggaatgtcaccttccaggtgactggctggctccaaaggcctttgcatgatctccaggagtttggaggggagaggccacattccaaatccagcttgaaaagtgctctgtatcaccctcagcactgagggggccagagtctaggaggaaggaggcacagggttggggggcagccctgacctggtggccgcacctgccaggtcccgagagacaacccatctcacacacattcaaaaacacacaccagggagcacatggctaaacaaatcgcactaaacgccaggaaggcag

SEQ ID NO: 60 - DNA, 700 bp, Homo sapiens
ctgccttcctggcgtttagtgcgatttgtttagccatgtgctccctggtgtgtgtttttgaatgtgtgtgagatgggttgtctctcgggacctggcaggtgcggccaccaggtcagggctgccccccaaccctgtgcctccttcctcctagactctggccccctcagtgctgagggtgatacagagcacttttcaagctggatttggaatgtggcctctcccctccaaactcctggagatcatgcaaaggcctttggagccagccagtcacctggaaggtgacattcccaccagctgaggcctcaccttcagcgggggctgggcagctttggagcctggggccagccaagctcactctgcccatatccctgccacgtgtggcccagcggatgatcacctgtcttcatctgcgtactgggccacatccctcctgccgtcccccacttccctgatgacacctacagcaagcccctacccaagtgttctgtgatcccctgtaaatgtggcctccctagctacttgcttttatgaaaccaacaatcctggggacacagttttcggctgtctcaagacggggcaaccactcttttccccaggcctgtgggtcccaggcctggagctagggttggcattcttgcctgaattctccactctatcccaacccctgaggccgcctgaggaggctcagactgtgtcaggc

SEQ ID NO: 61 - DNA, 6374 bp, Artificial Sequence
  Synthetic
ttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagcggatccgccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttacccatacgatgttccagattacgcttaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcctt

SEQ ID NO: 62 - DNA, 6347 bp, Artificial Sequence
  Synthetic
cagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagcggatccgccaccatggattggggcacactccagagcatcctcgggggtgtcaacaaacactccaccagcattggaaagatctggctcacggtcctcttcatcttccgcatcatgatcctcgtggtggctgcaaaggaggtgtggggagatgagcaagccgattttgtctgcaacacgctccagcctggctgcaagaatgtatgctacgaccaccacttccccatctctcacatccggctctgggctctgcagctgatcatggtgtccacgccagccctcctggtagctatgcatgtggcctaccggagacatgaaaagaaacggaagttcatgaagggagagataaagaacgagtttaaggacatcgaagagatcaaaacccagaaggtccgtatcgaagggtccctgtggtggacctacaccaccagcatcttcttccgggtcatctttgaagccgtcttcatgtacgtcttttacatcatgtacaatggcttcttcatgcaacgtctggtgaaatgcaacgcttggccctgccccaatacagtggactgcttcatttccaggcccacagaaaagactgtcttcaccgtgtttatgatttctgtgtctggaatttgcattctgctaaatatcacagagctgtgctatttgttcgttaggtattgctcaggaaagtccaaaagaccagtctaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattg

SEQ ID NO: 63 - DNA, 6347 bp, Artificial Sequence
  Synthetic
cagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagcggatccgccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattg

SEQ ID NO: 64 - DNA, 7150 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaagagagcacttgggaagagcccccgagggcagccggggcttgccgcctcacccttttggtttcacatcccagaaatcagtaaggcaggaattggaggctgcttcttgccttagcaactcggtgaccttaggcagaacagttcagccttctgagtgtccttcctcttctgtaaggggagcgtaaaccgtcctccatgcagaacgtgtactgtgcctggcacagcactggggcattaggatctccaaattaaaggctcactctgcgggatggaggcagccacagctggaagaaggaacatttggggccagaagtccccctacctccgtcctaagagagaagatgggaataacgaccctcgctgaaatgattgctctctggccagctcgcctcgcatccacatccaaatctgggaggcacagagcgcatcaggacatcgggttctgtcagtgtaatgggcgtggctcctgaccttctgtctgtatcagagaagataagggagaacatttgaaagaaaggagaaagaagatagccactggagaacagagcaaaggagccagcagaaaaagacgagacggctgtagccccacaggaagcagaaaccgataggctaagtaggatacacacaaagaaaagtagatcccgagaggcatttccccgagggctttcatgtggtttctcgtgaggagaagctgactgcagggtgtttgaaagaacgacttatgcagccataaaaaatgatgagttcatgtcctttgtagggacatggatgattaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 65 - DNA, 7108 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaagagagcacttgggaagagcccccgagggcagccggggcttgccgcctcacccttttggtttcacatcccagaaatcagtaaggcaggaattggaggctgcttcttgccttagcaactcggtgaccttaggcagaacagttcagccttctgagtgtccttcctcttctgtaaggggagcgtaaaccgtcctccatgcagaacgtgtactgtgcctggcacagcactggggcattaggatctccaaattaaaggctcactctgcgggatggaggcagccacagctggaagaaggaacatttggggccagaagtccccctacctccgtcctaagagagaagatgggaataacgaccctcgctgaaatgattgctctctggccagctcgcctcgcatccacatccaaatctgggaggcacagagcgcatcaggacatcgggttctgtcagtgtaatgggcgtggctcctgaccttctgtctgtatcagagaagataagggagaacatttgaaagaaaggagaaagaagatagccactggagaacagagcaaaggagccagcagaaaaagacgagacggctgtagccccacaggaagcagaaaccgataggctaagtaggatacacacaaagaaaagtagatcccgagaggcatttccccgagggctttcatgtggtttctcgtgaggagaagctgactgcagggtgtttgaaagaacgacttatgcagccataaaaaatgatgagttcatgtcctttgtagggacatggatgattaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 66 - DNA, 7135 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaagagagcacttgggaagagcccccgagggcagccggggcttgccgcctcacccttttggtttcacatcccagaaatcagtaaggcaggaattggaggctgcttcttgccttagcaactcggtgaccttaggcagaacagttcagccttctgagtgtccttcctcttctgtaaggggagcgtaaaccgtcctccatgcagaacgtgtactgtgcctggcacagcactggggcattaggatctccaaattaaaggctcactctgcgggatggaggcagccacagctggaagaaggaacatttggggccagaagtccccctacctccgtcctaagagagaagatgggaataacgaccctcgctgaaatgattgctctctggccagctcgcctcgcatccacatccaaatctgggaggcacagagcgcatcaggacatcgggttctgtcagtgtaatgggcgtggctcctgaccttctgtctgtatcagagaagataagggagaacatttgaaagaaaggagaaagaagatagccactggagaacagagcaaaggagccagcagaaaaagacgagacggctgtagccccacaggaagcagaaaccgataggctaagtaggatacacacaaagaaaagtagatcccgagaggcatttccccgagggctttcatgtggtttctcgtgaggagaagctgactgcagggtgtttgaaagaacgacttatgcagccataaaaaatgatgagttcatgtcctttgtagggacatggatgattaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggattggggcacactccagagcatcctcgggggtgtcaacaaacactccaccagcattggaaagatctggctcacggtcctcttcatcttccgcatcatgatcctcgtggtggctgcaaaggaggtgtggggagatgagcaagccgattttgtctgcaacacgctccagcctggctgcaagaatgtatgctacgaccaccacttccccatctctcacatccggctctgggctctgcagctgatcatggtgtccacgccagccctcctggtagctatgcatgtggcctaccggagacatgaaaagaaacggaagttcatgaagggagagataaagaacgagtttaaggacatcgaagagatcaaaacccagaaggtccgtatcgaagggtccctgtggtggacctacaccaccagcatcttcttccgggtcatctttgaagccgtcttcatgtacgtcttttacatcatgtacaatggcttcttcatgcaacgtctggtgaaatgcaacgcttggccctgccccaatacagtggactgcttcatttccaggcccacagaaaagactgtcttcaccgtgtttatgatttctgtgtctggaatttgcattctgctaaatatcacagagctgtgctatttgttcgttaggtattgctcaggaaagtccaaaagaccagtctacccatacgatgttccagattacgcttaaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 67 - DNA, 7124 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcagtgatgcctgaaacctcagatggtactgaaccctctatataatctgttttttcctatacatacaaacctaccataaggcttaatggtaagagattaacaataaagaataataaaacaacacttataacaatgtataacaatatattgtaatataagtttttggatgcagtctctctctcaaaatgctatcatattttccaactgtggttgactacaggtaactggaaccacaaaaatgaaacagtggataagagggcgactcctgtaccaaagaaaaaaatagagtgttgcagctgtaacatagttgaatgactgagttagactgcataactgacacacaaaaccacataaatataaatgaaggaatctctgggtgtaatctggtgcaaaggtgactgtgttaatcattaatccacaagttgctatcctgaagtgtgccaaatgctttatgtttatttcatcacatagctctataaagaaaggatttgtaattcctttctacagaagtggaaagtaagtcttaagactcaaaaaactttaaaaactacaatgaagtaacaacttttattaatttattttgtgtctttccagaatttctatatatataggaatgtgatatgaatctatatgtgaattgaatctacatgaatattgatgacttttatttccccttttgcacataagatagaatattttacctactattccacactttgcttttcttaacatatcatgggatctttttatataagtgaacaaagagtttcttcattctttcacacagtttaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 68 - DNA, 7151 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcagtgatgcctgaaacctcagatggtactgaaccctctatataatctgttttttcctatacatacaaacctaccataaggcttaatggtaagagattaacaataaagaataataaaacaacacttataacaatgtataacaatatattgtaatataagtttttggatgcagtctctctctcaaaatgctatcatattttccaactgtggttgactacaggtaactggaaccacaaaaatgaaacagtggataagagggcgactcctgtaccaaagaaaaaaatagagtgttgcagctgtaacatagttgaatgactgagttagactgcataactgacacacaaaaccacataaatataaatgaaggaatctctgggtgtaatctggtgcaaaggtgactgtgttaatcattaatccacaagttgctatcctgaagtgtgccaaatgctttatgtttatttcatcacatagctctataaagaaaggatttgtaattcctttctacagaagtggaaagtaagtcttaagactcaaaaaactttaaaaactacaatgaagtaacaacttttattaatttattttgtgtctttccagaatttctatatatataggaatgtgatatgaatctatatgtgaattgaatctacatgaatattgatgacttttatttccccttttgcacataagatagaatattttacctactattccacactttgcttttcttaacatatcatgggatctttttatataagtgaacaaagagtttcttcattctttcacacagtttaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggattggggcacactccagagcatcctcgggggtgtcaacaaacactccaccagcattggaaagatctggctcacggtcctcttcatcttccgcatcatgatcctcgtggtggctgcaaaggaggtgtggggagatgagcaagccgattttgtctgcaacacgctccagcctggctgcaagaatgtatgctacgaccaccacttccccatctctcacatccggctctgggctctgcagctgatcatggtgtccacgccagccctcctggtagctatgcatgtggcctaccggagacatgaaaagaaacggaagttcatgaagggagagataaagaacgagtttaaggacatcgaagagatcaaaacccagaaggtccgtatcgaagggtccctgtggtggacctacaccaccagcatcttcttccgggtcatctttgaagccgtcttcatgtacgtcttttacatcatgtacaatggcttcttcatgcaacgtctggtgaaatgcaacgcttggccctgccccaatacagtggactgcttcatttccaggcccacagaaaagactgtcttcaccgtgtttatgatttctgtgtctggaatttgcattctgctaaatatcacagagctgtgctatttgttcgttaggtattgctcaggaaagtccaaaagaccagtctacccatacgatgttccagattacgcttaaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 69 - skipped

SEQ ID NO: 70 - DNA, 7208 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgctatctatcatcttgaagggcttctggaacaagttagaatagagtcaacactcatgaactgctgtagcaaaaaaaactatagatgtaggattgacaagggcaatagagcgatgactccctggctgtgttgtatttgatggacggcagtagcttttcacaaaatgctcatttggatgtttcaaattaaaacgtttcactttctagaaccaattacgtggtcagtttagctcctgaggtcccagtcagaggggtattctgtagcttgcaaagcctctctttggggactggacatggagtctgtggtcttagaattcagaaccgggagaatgtgttagccactcatctaagctattccttaaacgctttcagagccatctccactgtggggaaagaagttctttgtgttctctgacttagtctcattctaaaaaaaaaaaaaaaaaaaaaaaaaaagcaattgcaatacccagagcgcacagtagatggcactgagacttgtcggaaagctggacgcactcaagaggtggcagaaaaatctataggtaagcttttcttctagtctggtgttgctgctcctgaccttattaatgggctgagaaatagatttctttcctttccttttcttttttatatgaaattaaatgaagtataaaagaatatgagaatgtgttgctattagcaaggataagtaatgctttaggaaacgtttggttcatgtgtgtgttttcagactgatgtgtgtcctggatccagtgtaaaatgtacttctgtctgtaggtctctgccacagaaaagttggaaagccattgttgtattccatttccagggcaacaaaagataccactgtcacttcatgtgaaatggtgttgtttaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 71 - DNA, 7235 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgctatctatcatcttgaagggcttctggaacaagttagaatagagtcaacactcatgaactgctgtagcaaaaaaaactatagatgtaggattgacaagggcaatagagcgatgactccctggctgtgttgtatttgatggacggcagtagcttttcacaaaatgctcatttggatgtttcaaattaaaacgtttcactttctagaaccaattacgtggtcagtttagctcctgaggtcccagtcagaggggtattctgtagcttgcaaagcctctctttggggactggacatggagtctgtggtcttagaattcagaaccgggagaatgtgttagccactcatctaagctattccttaaacgctttcagagccatctccactgtggggaaagaagttctttgtgttctctgacttagtctcattctaaaaaaaaaaaaaaaaaaaaaaaaaaagcaattgcaatacccagagcgcacagtagatggcactgagacttgtcggaaagctggacgcactcaagaggtggcagaaaaatctataggtaagcttttcttctagtctggtgttgctgctcctgaccttattaatgggctgagaaatagatttctttcctttccttttcttttttatatgaaattaaatgaagtataaaagaatatgagaatgtgttgctattagcaaggataagtaatgctttaggaaacgtttggttcatgtgtgtgttttcagactgatgtgtgtcctggatccagtgtaaaatgtacttctgtctgtaggtctctgccacagaaaagttggaaagccattgttgtattccatttccagggcaacaaaagataccactgtcacttcatgtgaaatggtgttgtttaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggattggggcacactccagagcatcctcgggggtgtcaacaaacactccaccagcattggaaagatctggctcacggtcctcttcatcttccgcatcatgatcctcgtggtggctgcaaaggaggtgtggggagatgagcaagccgattttgtctgcaacacgctccagcctggctgcaagaatgtatgctacgaccaccacttccccatctctcacatccggctctgggctctgcagctgatcatggtgtccacgccagccctcctggtagctatgcatgtggcctaccggagacatgaaaagaaacggaagttcatgaagggagagataaagaacgagtttaaggacatcgaagagatcaaaacccagaaggtccgtatcgaagggtccctgtggtggacctacaccaccagcatcttcttccgggtcatctttgaagccgtcttcatgtacgtcttttacatcatgtacaatggcttcttcatgcaacgtctggtgaaatgcaacgcttggccctgccccaatacagtggactgcttcatttccaggcccacagaaaagactgtcttcaccgtgtttatgatttctgtgtctggaatttgcattctgctaaatatcacagagctgtgctatttgttcgttaggtattgctcaggaaagtccaaaagaccagtctacccatacgatgttccagattacgcttaaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 72 - DNA, 7262 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaactgggcaatgcgttaaactggcttttttgacttcccagaacaatatctaattagcaaataacacaattcagtgacattcagcaggatgcaaattccagacactgcaatcatgaacactgtgaagacagtcttctccgtgggccgggacacaaagcagtccacagtgttgggacaaggccaggcgttgcacttcaccagccgctgcatggagaagccgtcgtacatgacatagaagacgtacatgaaggcggcttcgaagatgacccggaagaagatgctgcttgtgtaggtccaccacagggagccttcgatgcggaccttctgggttttgatctcctcgatgtccttaaattcactctttatctcccccttgatgaacttcctcttcttctcatgtctccggtaggccacgtgcatggccactaggagcgctggcgtggacacgaagatcagctgcagggcccatagccggatgtgggagatggggaagtagtgatcgtagcacacgttcttgcagcctggctgcagggtgttgcagacaaagtcggcctgctcatctccccacacctcctttgcagccacaacgaggatcataatgcgaaaaatgaagaggacggtgagccagatctttccaatgctggtggagtgtttgttcacaccccccaggatcgtctgcagcgtgccccaatccatcttctactctgggcggtttgctctggaaaagacgaatgcacacaacacaggaatcactagctaggacagaacagggagacttctctgagtctgggtaagcaagcatgcttaaatctcttcctgagcaaacaccaactcttacacaacctcaccaaaacaggtgaagacagaaccaacttagtttgtcattaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 73 - DNA, 7220 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaactgggcaatgcgttaaactggcttttttgacttcccagaacaatatctaattagcaaataacacaattcagtgacattcagcaggatgcaaattccagacactgcaatcatgaacactgtgaagacagtcttctccgtgggccgggacacaaagcagtccacagtgttgggacaaggccaggcgttgcacttcaccagccgctgcatggagaagccgtcgtacatgacatagaagacgtacatgaaggcggcttcgaagatgacccggaagaagatgctgcttgtgtaggtccaccacagggagccttcgatgcggaccttctgggttttgatctcctcgatgtccttaaattcactctttatctcccccttgatgaacttcctcttcttctcatgtctccggtaggccacgtgcatggccactaggagcgctggcgtggacacgaagatcagctgcagggcccatagccggatgtgggagatggggaagtagtgatcgtagcacacgttcttgcagcctggctgcagggtgttgcagacaaagtcggcctgctcatctccccacacctcctttgcagccacaacgaggatcataatgcgaaaaatgaagaggacggtgagccagatctttccaatgctggtggagtgtttgttcacaccccccaggatcgtctgcagcgtgccccaatccatcttctactctgggcggtttgctctggaaaagacgaatgcacacaacacaggaatcactagctaggacagaacagggagacttctctgagtctgggtaagcaagcatgcttaaatctcttcctgagcaaacaccaactcttacacaacctcaccaaaacaggtgaagacagaaccaacttagtttgtcattaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 74 - DNA, 7247 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaactgggcaatgcgttaaactggcttttttgacttcccagaacaatatctaattagcaaataacacaattcagtgacattcagcaggatgcaaattccagacactgcaatcatgaacactgtgaagacagtcttctccgtgggccgggacacaaagcagtccacagtgttgggacaaggccaggcgttgcacttcaccagccgctgcatggagaagccgtcgtacatgacatagaagacgtacatgaaggcggcttcgaagatgacccggaagaagatgctgcttgtgtaggtccaccacagggagccttcgatgcggaccttctgggttttgatctcctcgatgtccttaaattcactctttatctcccccttgatgaacttcctcttcttctcatgtctccggtaggccacgtgcatggccactaggagcgctggcgtggacacgaagatcagctgcagggcccatagccggatgtgggagatggggaagtagtgatcgtagcacacgttcttgcagcctggctgcagggtgttgcagacaaagtcggcctgctcatctccccacacctcctttgcagccacaacgaggatcataatgcgaaaaatgaagaggacggtgagccagatctttccaatgctggtggagtgtttgttcacaccccccaggatcgtctgcagcgtgccccaatccatcttctactctgggcggtttgctctggaaaagacgaatgcacacaacacaggaatcactagctaggacagaacagggagacttctctgagtctgggtaagcaagcatgcttaaatctcttcctgagcaaacaccaactcttacacaacctcaccaaaacaggtgaagacagaaccaacttagtttgtcattaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggattggggcacactccagagcatcctcgggggtgtcaacaaacactccaccagcattggaaagatctggctcacggtcctcttcatcttccgcatcatgatcctcgtggtggctgcaaaggaggtgtggggagatgagcaagccgattttgtctgcaacacgctccagcctggctgcaagaatgtatgctacgaccaccacttccccatctctcacatccggctctgggctctgcagctgatcatggtgtccacgccagccctcctggtagctatgcatgtggcctaccggagacatgaaaagaaacggaagttcatgaagggagagataaagaacgagtttaaggacatcgaagagatcaaaacccagaaggtccgtatcgaagggtccctgtggtggacctacaccaccagcatcttcttccgggtcatctttgaagccgtcttcatgtacgtcttttacatcatgtacaatggcttcttcatgcaacgtctggtgaaatgcaacgcttggccctgccccaatacagtggactgcttcatttccaggcccacagaaaagactgtcttcaccgtgtttatgatttctgtgtctggaatttgcattctgctaaatatcacagagctgtgctatttgttcgttaggtattgctcaggaaagtccaaaagaccagtctacccatacgatgttccagattacgcttaaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 75 - DNA, 7204 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaagctactaactacaaccacgagattatagatgtttgctgatattgttctcagtttggttattgtgttgtttatgaatgaaagtagtgtatgtttgtgtgaatttttgtttttaattttttatgagtgccctaacaaagattacaaattgggaatacaaactccagagcaatggagacagtgacacttttgtggaggggtacatgtggctgttcgggtggttattaacacaggctgctgcccctgccctgcaatgggaatccccagggcattggaggattcaacctcttgcagttacctcttgtaagacagcagatggcagcagagagaggctttgcacatccctgcaggttctagtttgcacaaagggcttctgagagacctatcaaccaattataacatcaagtggcaaaaagagtccttgataagttatttcgcttctcaaagaaaccgaaaacgccaaactaatcactagtcttgtttttttttttcctggcaaaagcctgctatctttcatgatttagctttcatgaaattgttcctgaagacccccaaaagaaacaatttcatgccccgaactctgttcagagactttgctgtgcctgtcatgtccagcttgccatatcctgttttgtaaagtagccaccttatatacacacctgctgtctgcactgtgacctcctttcaaaatcatctttggttcttcagaggcctggaataatgctctgcccagatgaagatctccgtaaatgtgtttttgaaatggctaatcaaataatggatacccttaggtatttttgcagaaacacttggcagccttccataatatccctactatgaaatggaaacttgtgaatgagatgtggctttaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 76 - DNA, 7231 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaagctactaactacaaccacgagattatagatgtttgctgatattgttctcagtttggttattgtgttgtttatgaatgaaagtagtgtatgtttgtgtgaatttttgtttttaattttttatgagtgccctaacaaagattacaaattgggaatacaaactccagagcaatggagacagtgacacttttgtggaggggtacatgtggctgttcgggtggttattaacacaggctgctgcccctgccctgcaatgggaatccccagggcattggaggattcaacctcttgcagttacctcttgtaagacagcagatggcagcagagagaggctttgcacatccctgcaggttctagtttgcacaaagggcttctgagagacctatcaaccaattataacatcaagtggcaaaaagagtccttgataagttatttcgcttctcaaagaaaccgaaaacgccaaactaatcactagtcttgtttttttttttcctggcaaaagcctgctatctttcatgatttagctttcatgaaattgttcctgaagacccccaaaagaaacaatttcatgccccgaactctgttcagagactttgctgtgcctgtcatgtccagcttgccatatcctgttttgtaaagtagccaccttatatacacacctgctgtctgcactgtgacctcctttcaaaatcatctttggttcttcagaggcctggaataatgctctgcccagatgaagatctccgtaaatgtgtttttgaaatggctaatcaaataatggatacccttaggtatttttgcagaaacacttggcagccttccataatatccctactatgaaatggaaacttgtgaatgagatgtggctttaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggattggggcacactccagagcatcctcgggggtgtcaacaaacactccaccagcattggaaagatctggctcacggtcctcttcatcttccgcatcatgatcctcgtggtggctgcaaaggaggtgtggggagatgagcaagccgattttgtctgcaacacgctccagcctggctgcaagaatgtatgctacgaccaccacttccccatctctcacatccggctctgggctctgcagctgatcatggtgtccacgccagccctcctggtagctatgcatgtggcctaccggagacatgaaaagaaacggaagttcatgaagggagagataaagaacgagtttaaggacatcgaagagatcaaaacccagaaggtccgtatcgaagggtccctgtggtggacctacaccaccagcatcttcttccgggtcatctttgaagccgtcttcatgtacgtcttttacatcatgtacaatggcttcttcatgcaacgtctggtgaaatgcaacgcttggccctgccccaatacagtggactgcttcatttccaggcccacagaaaagactgtcttcaccgtgtttatgatttctgtgtctggaatttgcattctgctaaatatcacagagctgtgctatttgttcgttaggtattgctcaggaaagtccaaaagaccagtctacccatacgatgttccagattacgcttaaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 77 - DNA, 7214 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtctcagctggagtgacgcacctcatccatgcgggcctggcgtctggaaggtggctgggtctctcgggcttgagcaccatcatcttagctccaacatgtcattattccttcctcactgaggacttttctgcttcctaattggttgttgaagatgaggcccccatgctcttttaagaaaacctgttgtgccccaggcttggctgtgatgggcactgactcatacagaagtagaaaggcctgctgagtcatcaacactcgtgcgacgccctcgcattttcattaatgatggcctccctgccacacgtgaatcactccagcccgagatctgaaaccaggacacaccccaggggcgaggtgacgctgagtgagcccagctgtgtccctttcatgagaactcagagcacagggctctgtgtgcatggccgtcccctccagagaggaggaagtaaatgccgggattagtggaagatcatttccttctatttgccttggcttacgtctttcagaattcaaacacgtgcactgttgaccctgcaatggtggagtttttggattttccttcagtccgattgctaaaatacttccctctcatgtgagctgttgtgaaagtcatcagccagataccattctaaaaacaaagaatgtgcttctcgtatgttgcatgctggttactgaaatattagggaattacataaaggttttctggggcacatattcaagctgaatgataaaattgaaggtcacacaaagctaaggtctttcaaatcctgacccaattagctctctgttagctctctgactttggacaagctgtctggtcctctgaagcatactttgttcgccctgggtaggggccctctgttttaacagcgtttggcattaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 78 - DNA, 7241 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtctcagctggagtgacgcacctcatccatgcgggcctggcgtctggaaggtggctgggtctctcgggcttgagcaccatcatcttagctccaacatgtcattattccttcctcactgaggacttttctgcttcctaattggttgttgaagatgaggcccccatgctcttttaagaaaacctgttgtgccccaggcttggctgtgatgggcactgactcatacagaagtagaaaggcctgctgagtcatcaacactcgtgcgacgccctcgcattttcattaatgatggcctccctgccacacgtgaatcactccagcccgagatctgaaaccaggacacaccccaggggcgaggtgacgctgagtgagcccagctgtgtccctttcatgagaactcagagcacagggctctgtgtgcatggccgtcccctccagagaggaggaagtaaatgccgggattagtggaagatcatttccttctatttgccttggcttacgtctttcagaattcaaacacgtgcactgttgaccctgcaatggtggagtttttggattttccttcagtccgattgctaaaatacttccctctcatgtgagctgttgtgaaagtcatcagccagataccattctaaaaacaaagaatgtgcttctcgtatgttgcatgctggttactgaaatattagggaattacataaaggttttctggggcacatattcaagctgaatgataaaattgaaggtcacacaaagctaaggtctttcaaatcctgacccaattagctctctgttagctctctgactttggacaagctgtctggtcctctgaagcatactttgttcgccctgggtaggggccctctgttttaacagcgtttggcattaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggattggggcacactccagagcatcctcgggggtgtcaacaaacactccaccagcattggaaagatctggctcacggtcctcttcatcttccgcatcatgatcctcgtggtggctgcaaaggaggtgtggggagatgagcaagccgattttgtctgcaacacgctccagcctggctgcaagaatgtatgctacgaccaccacttccccatctctcacatccggctctgggctctgcagctgatcatggtgtccacgccagccctcctggtagctatgcatgtggcctaccggagacatgaaaagaaacggaagttcatgaagggagagataaagaacgagtttaaggacatcgaagagatcaaaacccagaaggtccgtatcgaagggtccctgtggtggacctacaccaccagcatcttcttccgggtcatctttgaagccgtcttcatgtacgtcttttacatcatgtacaatggcttcttcatgcaacgtctggtgaaatgcaacgcttggccctgccccaatacagtggactgcttcatttccaggcccacagaaaagactgtcttcaccgtgtttatgatttctgtgtctggaatttgcattctgctaaatatcacagagctgtgctatttgttcgttaggtattgctcaggaaagtccaaaagaccagtctacccatacgatgttccagattacgcttaaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 79 - DNA, 7251 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcgacctgaacgattaaggcaaaacttcgaaatgtgccccagcagagatttatttttcagggggtgttttgcattccagcccctctgccttcctggcgtttagtgcgatttgtttagccatgtgctccctggtgtgtgtttttgaatgtgtgtgagatgggttgtctctcgggacctggcaggtgcggccaccaggtcagggctgccccccaaccctgtgcctccttcctcctagactctggccccctcagtgctgagggtgatacagagcacttttcaagctggatttggaatgtggcctctcccctccaaactcctggagatcatgcaaaggcctttggagccagccagtcacctggaaggtgacattcccaccagctgaggcctcaccttcagcgggggctgggcagctttggagcctggggccagccaagctcactctgcccatatccctgccacgtgtggcccagcggatgatcacctgtcttcatctgcgtactgggccacatccctcctgccgtcccccacttccctgatgacacctacagcaagcccctacccaagtgttctgtgatcccctgtaaatgtggcctccctagctacttgcttttatgaaaccaacaatcctggggacacagttttcggctgtctcaagacggggcaaccactcttttccccaggcctgtgggtcccaggcctggagctagggttggcattcttgcctgaattctccactctatcccaacccctgaggccgcctgaggaggctcagactgtgtcaggctaggaggacagtcaaaccacaaaaacatgccttttaagaagtataagcacaaatccctctttgatgttatataaaagctcagtgtcactttaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 80 - DNA, 7209 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcgacctgaacgattaaggcaaaacttcgaaatgtgccccagcagagatttatttttcagggggtgttttgcattccagcccctctgccttcctggcgtttagtgcgatttgtttagccatgtgctccctggtgtgtgtttttgaatgtgtgtgagatgggttgtctctcgggacctggcaggtgcggccaccaggtcagggctgccccccaaccctgtgcctccttcctcctagactctggccccctcagtgctgagggtgatacagagcacttttcaagctggatttggaatgtggcctctcccctccaaactcctggagatcatgcaaaggcctttggagccagccagtcacctggaaggtgacattcccaccagctgaggcctcaccttcagcgggggctgggcagctttggagcctggggccagccaagctcactctgcccatatccctgccacgtgtggcccagcggatgatcacctgtcttcatctgcgtactgggccacatccctcctgccgtcccccacttccctgatgacacctacagcaagcccctacccaagtgttctgtgatcccctgtaaatgtggcctccctagctacttgcttttatgaaaccaacaatcctggggacacagttttcggctgtctcaagacggggcaaccactcttttccccaggcctgtgggtcccaggcctggagctagggttggcattcttgcctgaattctccactctatcccaacccctgaggccgcctgaggaggctcagactgtgtcaggctaggaggacagtcaaaccacaaaaacatgccttttaagaagtataagcacaaatccctctttgatgttatataaaagctcagtgtcactttaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 81 - DNA, 7236 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcgacctgaacgattaaggcaaaacttcgaaatgtgccccagcagagatttatttttcagggggtgttttgcattccagcccctctgccttcctggcgtttagtgcgatttgtttagccatgtgctccctggtgtgtgtttttgaatgtgtgtgagatgggttgtctctcgggacctggcaggtgcggccaccaggtcagggctgccccccaaccctgtgcctccttcctcctagactctggccccctcagtgctgagggtgatacagagcacttttcaagctggatttggaatgtggcctctcccctccaaactcctggagatcatgcaaaggcctttggagccagccagtcacctggaaggtgacattcccaccagctgaggcctcaccttcagcgggggctgggcagctttggagcctggggccagccaagctcactctgcccatatccctgccacgtgtggcccagcggatgatcacctgtcttcatctgcgtactgggccacatccctcctgccgtcccccacttccctgatgacacctacagcaagcccctacccaagtgttctgtgatcccctgtaaatgtggcctccctagctacttgcttttatgaaaccaacaatcctggggacacagttttcggctgtctcaagacggggcaaccactcttttccccaggcctgtgggtcccaggcctggagctagggttggcattcttgcctgaattctccactctatcccaacccctgaggccgcctgaggaggctcagactgtgtcaggctaggaggacagtcaaaccacaaaaacatgccttttaagaagtataagcacaaatccctctttgatgttatataaaagctcagtgtcactttaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggattggggcacactccagagcatcctcgggggtgtcaacaaacactccaccagcattggaaagatctggctcacggtcctcttcatcttccgcatcatgatcctcgtggtggctgcaaaggaggtgtggggagatgagcaagccgattttgtctgcaacacgctccagcctggctgcaagaatgtatgctacgaccaccacttccccatctctcacatccggctctgggctctgcagctgatcatggtgtccacgccagccctcctggtagctatgcatgtggcctaccggagacatgaaaagaaacggaagttcatgaagggagagataaagaacgagtttaaggacatcgaagagatcaaaacccagaaggtccgtatcgaagggtccctgtggtggacctacaccaccagcatcttcttccgggtcatctttgaagccgtcttcatgtacgtcttttacatcatgtacaatggcttcttcatgcaacgtctggtgaaatgcaacgcttggccctgccccaatacagtggactgcttcatttccaggcccacagaaaagactgtcttcaccgtgtttatgatttctgtgtctggaatttgcattctgctaaatatcacagagctgtgctatttgttcgttaggtattgctcaggaaagtccaaaagaccagtctacccatacgatgttccagattacgcttaaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 82 - DNA, 7018 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttctaggtagacaactaagatgttcatcttatggtttaatgtttagttgtaaaggttgtttgcttctcatttggttccaagaaagagtatttaggccaatttcagggagaaatatgtgtatagatatattcatatgtcaaactgattagtgctgaatgtcacatttccatattctaataacatttctagcaaagaagaggacacagtgaagagagaattgcccgcattgtcattgtctctttctgagcctagaacgcctaacacttgggtgtggagagactcagcctcaattcactttctagcagccactgagatgtgcttgcctggggtgccccctggcaggcagggctggaactgctttccagtacccacacggactgtgaacgaatctttctttgtgctttgtgtacagaatggaagttcaacaaatatttgttgaatgtgtatgtccttccaatacgcagcagcccagagcaaacgtggtaatcttgtgtgtgttcatgtgaaagcagaatttaatggtgcttttaagcaccaaagtttaagatgcacgagaaaactgtatctccattttttccttttcgtttacaattacttgtataagccaggcacggtggtggctcacgcctgtaatcccagcactttgggaggccgaggcgggcggatcacatgaggtcgggagttaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 83 - DNA, 7045 bp, Artificial Sequence
  Synthetic
cgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttctaggtagacaactaagatgttcatcttatggtttaatgtttagttgtaaaggttgtttgcttctcatttggttccaagaaagagtatttaggccaatttcagggagaaatatgtgtatagatatattcatatgtcaaactgattagtgctgaatgtcacatttccatattctaataacatttctagcaaagaagaggacacagtgaagagagaattgcccgcattgtcattgtctctttctgagcctagaacgcctaacacttgggtgtggagagactcagcctcaattcactttctagcagccactgagatgtgcttgcctggggtgccccctggcaggcagggctggaactgctttccagtacccacacggactgtgaacgaatctttctttgtgctttgtgtacagaatggaagttcaacaaatatttgttgaatgtgtatgtccttccaatacgcagcagcccagagcaaacgtggtaatcttgtgtgtgttcatgtgaaagcagaatttaatggtgcttttaagcaccaaagtttaagatgcacgagaaaactgtatctccattttttccttttcgtttacaattacttgtataagccaggcacggtggtggctcacgcctgtaatcccagcactttgggaggccgaggcgggcggatcacatgaggtcgggagttaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagccatggattggggcacactccagagcatcctcgggggtgtcaacaaacactccaccagcattggaaagatctggctcacggtcctcttcatcttccgcatcatgatcctcgtggtggctgcaaaggaggtgtggggagatgagcaagccgattttgtctgcaacacgctccagcctggctgcaagaatgtatgctacgaccaccacttccccatctctcacatccggctctgggctctgcagctgatcatggtgtccacgccagccctcctggtagctatgcatgtggcctaccggagacatgaaaagaaacggaagttcatgaagggagagataaagaacgagtttaaggacatcgaagagatcaaaacccagaaggtccgtatcgaagggtccctgtggtggacctacaccaccagcatcttcttccgggtcatctttgaagccgtcttcatgtacgtcttttacatcatgtacaatggcttcttcatgcaacgtctggtgaaatgcaacgcttggccctgccccaatacagtggactgcttcatttccaggcccacagaaaagactgtcttcaccgtgtttatgatttctgtgtctggaatttgcattctgctaaatatcacagagctgtgctatttgttcgttaggtattgctcaggaaagtccaaaagaccagtctacccatacgatgttccagattacgcttaaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcatcgataccgagcgctgctcgagagatctgtgatagcggccatcaagctggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacacca

SEQ ID NO: 84 - DNA, 700 bp, Cynomolgus macaque
atggcaccagcttttgaaaaaagaaaacctttttgctggtagtctggcaaggagacagaaaaaaaccactcacatctgcctccccaggctgggggctgggccggattttataaggatagggtaatgaggggtggtctgtttggatcttgcaatgaggtgctgctgggaggtgtgatctgattggatcctgccatggagtgatgccaaagctccatctgattggatcctggatcctgccgtgtgtgctctgcttcttaatgcaacccctgctcctcagtctgagcccttagattctgcccacggttgcacgcttggttcactttggcatgctcaggttacatgaccttcagcttggggtccatggcaactgaaaagcaactcacaacttcctttcataaaaattgaacctgactggtctggtgcagtcacaccagctctatcccattgatgacaggaccgcatcatggggattagagcagagaggtcatagtaactagcattttcaagagggcaccctgatgtctggatgaacttcagggcaacaaaatagcgggcaggtgagcagttgaagacacccagacactgggcctgaccaaggtggggtggtggggatggcacaggaggacacaggatgggaattaatgagggcaggggctttgtcttgctcactgataagtccatggcacatagagggtgatcg

SEQ ID NO: 85 - DNA, 700 bp, Cynomolgus macaque
cgatcaccctctatgtgccatggacttatcagtgagcaagacaaagcccctgccctcattaattcccatcctgtgtcctcctgtgccatccccaccaccccaccttggtcaggcccagtgtctgggtgtcttcaactgctcacctgcccgctattttgttgccctgaagttcatccagacatcagggtgccctcttgaaaatgctagttactatgacctctctgctctaatccccatgatgcggtcctgtcatcaatgggatagagctggtgtgactgcaccagaccagtcaggttcaatttttatgaaaggaagttgtgagttgcttttcagttgccatggaccccaagctgaaggtcatgtaacctgagcatgccaaagtgaaccaagcgtgcaaccgtgggcagaatctaagggctcagactgaggagcaggggttgcattaagaagcagagcacacacggcaggatccaggatccaatcagatggagctttggcatcactccatggcaggatccaatcagatcacacctcccagcagcacctcattgcaagatccaaacagaccacccctcattaccctatccttataaaatccggcccagcccccagcctggggaggcagatgtgagtggtttttttctgtctccttgccagactaccagcaaaaaggttttcttttttcaaaagctggtgccat

SEQ ID NO: 86 - DNA, 700 bp, Cynomolgus macaque
ccgttaggaaaagaaaaacagaaggaattgtgttctctggagggcagggctctgagtactgagtctcatgttttcaaagtcggaaagtgtccacagttaatatttggatgggcccacagtgcccgtcttgctcgccggagcccaggcctgtcccatcacagacaaagggctcttgctgtgcacctgtggagaggggagcttggctggggaaggcagggtcagcctctttgtgctctttttgtttgaagcagagttttgcaaagggagtggctctggaagaaaagcagagcgtggagtgtcagaggccggcgtgttgtgaaatgcataagccctggagaccctctgtaactggccttcacacacgcccgccgccaaggacaacactgaaccacggaagcggggtgtttgccagctcacgagacggggagacatgaagcttctaccagcagaggagctggaggggaaacagaaagaaagaactgagtctagcagcctccttggacatttcttccaacgcctccagcccagcacaacaaacaacctcagggcatccggcccgtgtcgcgccctggcacacccaactctgccctgctccaagagcccacagagggcctcggggtcacactcaaggagcatgcttggaatccaaagtgcatgctgtggtggggagatggacaaggacagaaatagcacccagcaa

SEQ ID NO: 87 - DNA, 700 bp, Cynomolgus macaque
ttgctgggtgctatttctgtccttgtccatctccccaccacagcatgcactttggattccaagcatgctccttgagtgtgaccccgaggccctctgtgggctcttggagcagggcagagttgggtgtgccagggcgcgacacgggccggatgccctgaggttgtttgttgtgctgggctggaggcgttggaagaaatgtccaaggaggctgctagactcagttctttctttctgtttcccctccagctcctctgctggtagaagcttcatgtctccccgtctcgtgagctggcaaacaccccgcttccgtggttcagtgttgtccttggcggcgggcgtgtgtgaaggccagttacagagggtctccagggcttatgcatttcacaacacgccggcctctgacactccacgctctgcttttcttccagagccactccctttgcaaaactctgcttcaaacaaaaagagcacaaagaggctgaccctgccttccccagccaagctcccctctccacaggtgcacagcaagagccctttgtctgtgatgggacaggcctgggctccggcgagcaagacgggcactgtgggcccatccaaatattaactgtggacactttccgactttgaaaacatgagactcagtactcagagccctgccctccagagaacacaattccttctgtttttcttttcctaacgg

SEQ ID NO: 88 - DNA, 510 bp, Cynomolgus macaque
aaaaaagaatcacaattgccaccaaggctctatgttttcgcaaaagtccagcatttaaaagaaacttcctgcatggcctacatctgctgattggtaatttgtcgttcaggttaaaaacaaaacaagcgggcattgttgtgatatcatccttgataacatcccaagaaaactctagagctggcaagagaggaaagcagataatggtcaaagctgtcatctgagttttaaaaacactgtgatttttcttttaaaggaacatcttcagtttccaaggccatacacacggctcctaactgcagcttaaaattttccactgggctcccttctgagaacaaacgctattcagtggcgagtgccggacaccactgcgctttcaaaggtggctgccagaggacactcaggacttcacagcagccggtaagccagactggggtcagtcactcccccatcagaattattttgtttctcctttgcttaggaaaggaaggattcctcagattggcatcccag

SEQ ID NO: 89 - DNA, 510 bp, Cynomolgus macaque
ctgggatgccaatctgaggaatccttcctttcctaagcaaaggagaaacaaaataattctgatgggggagtgactgaccccagtctggcttaccggctgctgtgaagtcctgagtgtcctctggcagccacctttgaaagcgcagtggtgtccggcactcgccactgaatagcgtttgttctcagaagggagcccagtggaaaattttaagctgcagttaggagccgtgtgtatggccttggaaactgaagatgttcctttaaaagaaaaatcacagtgtttttaaaactcagatgacagctttgaccattatctgctttcctctcttgccagctctagagttttcttgggatgttatcaaggatgatatcacaacaatgcccgcttgttttgtttttaacctgaacgacaaattaccaatcagcagatgtaggccatgcaggaagtttcttttaaatgctggacttttgcgaaaacatagagccttggtggcaattgtgattctttttt

SEQ ID NO: 90 - DNA, 643 bp, Cynomolgus macaque
ataatgagcaacataaggttaaaataacattgcaaccccatggaagcaagagaaatggaaattattaataaatggaccacatgtaagggaatgctgtggttctattgtagagattacagagagcaatttaggagagccaggcgctgggggcaagagggaaatgaaacgaaaaccgaagggatttgttcaggaagaaaaatgaaaacagataaaaggtgttcatttcaaagcttccctctttcccagcatttttctgaagtagagtttgaaaggaaagcaaaataactgcaaaccaatacagtggcacgagttcactgacgcagagctaggaacgacgtccagagatctccagccccgcctcccgttctgggtcacctggctccttgacagccctgaaaactgcctgtgcaaatctccaggcatgttatacccatgagcggggacgtgtggcaccgacaaagggacctgtacacctttgaagtatcctgggagaccagactcacattccacacacgctcacgagtcactgagcagccccattggaaatacgtggcaccgtctcattccatatttgaccaaaaccagtgtttacccagctcagccgatagtttcatttttttaaccaaacctaatgcagaatggc

SEQ ID NO: 91 - DNA, 643 bp, Cynomolgus macaque
gccattctgcattaggtttggttaaaaaaatgaaactatcggctgagctgggtaaacactggttttggtcaaatatggaatgagacggtgccacgtatttccaatggggctgctcagtgactcgtgagcgtgtgtggaatgtgagtctggtctcccaggatacttcaaaggtgtacaggtccctttgtcggtgccacacgtccccgctcatgggtataacatgcctggagatttgcacaggcagttttcagggctgtcaaggagccaggtgacccagaacgggaggcggggctggagatctctggacgtcgttcctagctctgcgtcagtgaactcgtgccactgtattggtttgcagttattttgctttcctttcaaactctacttcagaaaaatgctgggaaagagggaagctttgaaatgaacaccttttatctgttttcatttttcttcctgaacaaatcccttcggttttcgtttcatttccctcttgcccccagcgcctggctctcctaaattgctctctgtaatctctacaatagaaccacagcattcccttacatgtggtccatttattaataatttccatttctcttgcttccatggggttgcaatgttattttaaccttatgttgctcattat

SEQ ID NO: 92 - DNA, 542 bp, Cynomolgus macaque
cacgtcttgtaatttttttactgaatgttagacattgcatataaaagactatccaggagtgttttgtttttgttttttctagtgagtgcaagtcccttgctctctgccagttggctggaatgagaatctgatcagatttcatcaagagtcaggttgagctgagactgagcggtagtgttcactaaattgagtgcaccactgatatctaatggaaacaaggacattttactttgctcctcagcctaacctgaatttcctatgccaccactgtataatggctggtttctttggttctcctaatgtgtgagctggaagcaggttgagacatagatttcatatcattttggcttcccttgcatctaacatggctccacaattcaagcactatgaaattgtttaactgttttccagtcttgcctccacagccacttttgcagtaaaatcacggatgggggtgacgttgagccaaactatttttgcatttggtggacttctaaattccaatccagctccaaatcttttggcagatttttcttaaaggt

SEQ ID NO: 93 - DNA, 542 bp, Cynomolgus macaque
acctttaagaaaaatctgccaaaagatttggagctggattggaatttagaagtccaccaaatgcaaaaatagtttggctcaacgtcacccccatccgtgattttactgcaaaagtggctgtggaggcaagactggaaaacagttaaacaatttcatagtgcttgaattgtggagccatgttagatgcaagggaagccaaaatgatatgaaatctatgtctcaacctgcttccagctcacacattaggagaaccaaagaaaccagccattatacagtggtggcataggaaattcaggttaggctgaggagcaaagtaaaatgtccttgtttccattagatatcagtggtgcactcaatttagtgaacactaccgctcagtctcagctcaacctgactcttgatgaaatctgatcagattctcattccagccaactggcagagagcaagggacttgcactcactagaaaaaacaaaaacaaaacactcctggatagtcttttatatgcaatgtctaacattcagtaaaaaaattacaagacgtg

SEQ ID NO: 94 - DNA, 523 bp, Cynomolgus macaque
cggcagagacctacagaccaaagtacatttcacactggatccaggacacacatcagtctgaaagcacacacatgaaccaaacgtttcctaaagcattacttacccttgctaatagcaacacattctcatattcttttatacttcatttaatttcatttaaaaaagaaaaagataggaaagaaatctatttctccgcccattaataaggtcagacgcagcaacgctagactagaagaaaagtttacctactgatttttctcccacctcctgagtgcgcacagctttccgacaagtgtcagtgccatctactgtgcgctctgggtactgcaatagccttttttttttttttttttttttttagaatgagactaaatgagagaacacaaagaacttctttccccacagtggagatggctctgaaagcgtttaaggaatggcttagatgagtggctaacacattatcccagttctgaattctaagaccacagactccatgtccgatccccaaagagaggctttgcaa

SEQ ID NO: 95 - DNA, 523 bp, Cynomolgus macaque
ttgcaaagcctctctttggggatcggacatggagtctgtggtcttagaattcagaactgggataatgtgttagccactcatctaagccattccttaaacgctttcagagccatctccactgtggggaaagaagttctttgtgttctctcatttagtctcattctaaaaaaaaaaaaaaaaaaaaaaaaggctattgcagtacccagagcgcacagtagatggcactgacacttgtcggaaagctgtgcgcactcaggaggtgggagaaaaatcagtaggtaaacttttcttctagtctagcgttgctgcgtctgaccttattaatgggcggagaaatagatttctttcctatctttttcttttttaaatgaaattaaatgaagtataaaagaatatgagaatgtgttgctattagcaagggtaagtaatgctttaggaaacgtttggttcatgtgtgtgctttcagactgatgtgtgtcctggatccagtgtgaaatgtactttggtctgtaggtctctgccg

SEQ ID NO: 96 - DNA, 579 bp, Cynomolgus macaque
ggtgtgtatatcaggtggttactttacaaaacaggatgtggcaagctggacctgatagacacatcaaagcctctgaacagagttcagggcatgaaatggtttcttttgggggtcttcaggaacaatttcatgaaagctaaatcatgaaagatagcagacttttgccaggaaaaaaaaacaaaacaaaacgagactagtgattagtttggcgttttcggtttctttgagaagcgaaataacttatcaaggactctttgtgccgcttgatgttctaatcggttgatgggtctctcagaagccctttctgcaaactagaacctgcagggatgtgcaaagcctctctctgctgccatctgctgtcttacaagaggtcactgcgagaggctgaatcccccaatgccttggggattcccactgcagggcaggggcgccagcctgtgttacaaccacctgaacggccacgtggaccttccacaaaagtgtcactgtttccattgctctggtgtttgtattcccaatgtgtagtctttgttagggcactcacaaaaagttaaaaacaaaaattcacacaagcataca

SEQ ID NO: 97 - DNA, 579 bp, Cynomolgus macaque
tgtatgcttgtgtgaatttttgtttttaactttttgtgagtgccctaacaaagactacacattgggaatacaaacaccagagcaatggaaacagtgacacttttgtggaaggtccacgtggccgttcaggtggttgtaacacaggctggcgcccctgccctgcagtgggaatccccaaggcattgggggattcagcctctcgcagtgacctcttgtaagacagcagatggcagcagagagaggctttgcacatccctgcaggttctagtttgcagaaagggcttctgagagacccatcaaccgattagaacatcaagcggcacaaagagtccttgataagttatttcgcttctcaaagaaaccgaaaacgccaaactaatcactagtctcgttttgttttgtttttttttcctggcaaaagtctgctatctttcatgatttagctttcatgaaattgttcctgaagacccccaaaagaaaccatttcatgccctgaactctgttcagaggctttgatgtgtctatcaggtccagcttgccacatcctgttttgtaaagtaaccacctgatatacacacc

SEQ ID NO: 98 - DNA, 700 bp, Cynomolgus macaque
ggtcaggatttgaaagaccttagctttgtgtgaccttcagttttatcattcagtttgaatatgtgccccagaaaacctttatgtaatttcctaatatttcagtaacatatttcacaacatacaagcagcacattctctttttttagaatggtgtctcgctgatgactttgacgacagctcacgtgagagggaagtatttcagcaatcagaccgaaggagaatccaaaaaccccactattgcggggtcaagagtgcacgtgtttgaattctgaaagatgtaagccaaggcaaacagaaggaaatgatcttccactaatccctgcatttacttcctcctctctggaggggacggccacacacacagagccctgtgctctgacttctcctgaaggggacacagctgggctcactcagtgtcacctcgcccctggggtgtgcccgggtttcagatctcaggctggagtgattcacgtgtagcagggaggccgtcattaatgaaaatgcaggggcgtcgcgggagtgttgatgattcagcaggcctttctacttctctatgagtcagtacccgtcgcagccaagcctggggcagaacaggttttcttaaaagagcatgggggcctcgtcttcaacaaccaattaggaggcagaaaagtcctcagtgaggaaggaataatgacatgttggagctaagatgatggtg

SEQ ID NO: 99 - DNA, 700 bp, Cynomolgus macaque
caccatcatcttagctccaacatgtcattattccttcctcactgaggacttttctgcctcctaattggttgttgaagacgaggcccccatgctcttttaagaaaacctgttctgccccaggcttggctgcgacgggtactgactcatagagaagtagaaaggcctgctgaatcatcaacactcccgcgacgcccctgcattttcattaatgacggcctccctgctacacgtgaatcactccagcctgagatctgaaacccgggcacaccccaggggcgaggtgacactgagtgagcccagctgtgtccccttcaggagaagtcagagcacagggctctgtgtgtgtggccgtcccctccagagaggaggaagtaaatgcagggattagtggaagatcatttccttctgtttgccttggcttacatctttcagaattcaaacacgtgcactcttgaccccgcaatagtggggtttttggattctccttcggtctgattgctgaaatacttccctctcacgtgagctgtcgtcaaagtcatcagcgagacaccattctaaaaaaagagaatgtgctgcttgtatgttgtgaaatatgttactgaaatattaggaaattacataaaggttttctggggcacatattcaaactgaatgataaaactgaaggtcacacaaagctaaggtctttcaaatcctgacc

SEQ ID NO: 100 - DNA, 532 bp, Cynomolgus macaque
gttttttcatgcatcttaaactttggtgcttaaagaaaagcaccattaaatcctgctctcacacgaacacacacaagattaccacgtttgctctgggctgccgcgtataggaaggacatatacattcaataaatatttgttgaacttccattctgtacacaaagcacaaagaaagattcgttcacagtccgcgtgggtacaggaaagcagttccagccctgcctgccagggggcaccccaggcaagcacatctcagtggctgcaagaaagtcagcgagttgaggctgagtctctctctatacccaagtgttaggtgttctaggctcaaagagagacaatgacaatgcgggcaattctctcttcactgtgtccctttctttgctagaaatgttattagaatgtggaaatgtgacccgtcgattgagaattcagcactaatcagtttgacatatgagtatatctacatagacacatatttctccctgaaattgtcctaaacactgtcttccttgaaaccaaatgagaaggaaac

SEQ ID NO: 101 - DNA, 532 bp, Cynomolgus macaque
gtttccttctcatttggtttcaaggaagacagtgtttaggacaatttcagggagaaatatgtgtctatgtagatatactcatatgtcaaactgattagtgctgaattctcaatcgacgggtcacatttccacattctaataacatttctagcaaagaaagggacacagtgaagagagaattgcccgcattgtcattgtctctctttgagcctagaacacctaacacttgggtatagagagagactcagcctcaactcgctgactttcttgcagccactgagatgtgcttgcctggggtgccccctggcaggcagggctggaactgctttcctgtacccacgcggactgtgaacgaatctttctttgtgctttgtgtacagaatggaagttcaacaaatatttattgaatgtatatgtccttcctatacgcggcagcccagagcaaacgtggtaatcttgtgtgtgttcgtgtgagagcaggatttaatggtgcttttctttaagcaccaaagtttaagatgcatgaaaaaac

SEQ ID NO: 102 - DNA, 120 bp, Artificial Sequence
  Synthetic
gacctcgaaggggacttggggggttcggggctttcgggggcggtcgggggttcgcggacccgggaagctctgaggacccagaggccgggcgcgctccgcccgcggcgccgccccctccgt

SEQ ID NO: 103 - DNA, 228 bp, Artificial Sequence
  Synthetic
aactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcag

SEQ ID NO: 104 - DNA, 22 bp, Artificial Sequence
  Synthetic
agcaaaccgcccagagtagaag

SEQ ID NO: 105 - DNA, 370 bp, Artificial Sequence
  Synthetic
gacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaag

SEQ ID NO: 106 - DNA, 130 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 107 - DNA, 130 bp, Artificial Sequence
  Synthetic
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 108 - DNA, 602 bp, Artificial Sequence
  Synthetic
gataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggactag

SEQ ID NO: 109 - DNA, 237 bp, Artificial Sequence
  Synthetic
gtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 110 - DNA, 3493 bp, Artificial Sequence
  Synthetic
gacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagcggatccgccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttacccatacgatgttccagattacgcttaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgatgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggactaggaattcatcgataccgagcgctgctcgagagatctgtgatagcggccatcaagctgggtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 111 - DNA, 3918 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaacgcgtttaattaagacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagcggatccgccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttacccatacgatgttccagattacgcttaaggcgcgccacccctgcagggaattccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaatctaatatggttttaatagaacagcgatatcaagcttatcgatgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggactaggaattcatcgataccgagcgctgctcgagagatctgtgatagcggccatcaagctgggtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcggtaccaaacctaggtaatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

(19) United States
(12) Patent Application Publication
(10) Pub. No.: US 2023/0340038 A1
(43) Pub. Date: Oct. 26, 2023

(54) RECOMBINANT ADENO ASSOCIATED VIRUS (RAAV) ENCODING GJB2 AND USES THEREOF

(71) Applicant: President and Fellows of Harvard College, Cambridge, MA (US)

(72) Inventors: David P. Corey, Cambridge, MA (US); Kevin T. Booth, Cambridge, MA (US); Cole W. D. Peters, Cambridge, MA (US); Maryna V. Ivanchenko, Cambridge, MA (US); Michael E Greenberg, Cambridge, MA (US); Sinisa Hrvatin, Cambridge, MA (US); Mark Aurel Nagy, Cambridge, MA (US); Eric C. Griffith, Cambridge, MA (US)

(73) Assignee: President and Fellows of Harvard College, Cambridge, MA (US)

(21) Appl. No.: 18/025,749

(22) PCT Filed: Sep. 14, 2021
(86) PCT No.: PCT/US2021/050205

§ 371 Date: Mar. 10, 2023

Related U.S. Application Data
(60) Provisional application No. 63/078,233, filed on Sep. 14, 2020.

(51) Int. Cl. C07K 14/47; C12N 15/86

(52) U.S. Cl. CPC C07K 14/47; C12N 15/86; C12N 2750/14143; C12N 2830/50; C12N 2830/008

(57) ABSTRACT

The present disclosure, at least in part, relates to compositions (e.g., isolated nucleic acid and rAAVs) and methods for treating Non-syndromic hearing loss and deafness (DFNB1) by delivering gap junction beta 2 (GJB2) protein to inner ear cells that normally express GJB2 (e.g., fibrocytes and supporting cells of the organ of Corti and nearby regions). The isolated nucleic acid of the present disclosure comprises an expression cassette, wherein the expression cassette comprises a gap junction beta 2 (GJB2) gene regulatory element (GRE) (e.g., GJB2 enhancers, GJB2 promoters, GJB2 5′ UTR, and/or GJB2 3′ UTR), and a nucleotide sequence encoding a GJB2 protein.

### RELATED APPLICATIONS

This application claims priority under 35 U.S.C. § 119(e) to U.S. Provisional Application, U.S. Ser. No. 63/161,619, filed Mar. 16, 2021, and to U.S. Provisional Application, U.S. Ser. No. 63/078,233, filed Sep. 14, 2020, each of which is incorporated herein by reference.

### FEDERALLY SPONSORED RESEARCH

This invention was made with Government support under DA048787 awarded by the National Institutes of Health. The Government has certain rights in the invention.

### BACKGROUND

Loss of gap junction beta 2 (GJB2) expression in the inner ear underlies a disorder termed nonsyndromic Hearing Loss and Deafness, (DFNB1), characterized by recessive, mild-to-profound sensorineural hearing impairment. Many of these patients are born with profound hearing loss, which is probably irreversible even at birth. Two-thirds have some residual hearing at birth, and the majority of those lose hearing over the next few years. Therefore, these patients are potential candidates for treatment of DFNB1. Previous gene replacement therapy of GJB2 failed to rescue hearing even though gene addition of the GJB2 gene rescued cell survival and the gap junction network. Effective GJB2 gene replacement therapy for hearing rescuing has not been developed.

### SUMMARY

The present disclosure, at least in part, relates to an isolated nucleic acid comprising an expression cassette, wherein the expression cassette comprises a gap junction beta 2 (GJB2) gene regulatory element (GRE), and a nucleotide sequence encoding a GJB2 protein. In some embodiments, the expression cassette further comprises a promoter (e.g., GJB2 promoter). In some embodiments, the expression cassette is flanked by two adeno-associated virus (AAV) inverted terminal repeats (ITRs). The presence of native GJB2 regulatory elements (GREs) in the isolated nucleic acid prevents promiscuous GJB2 gene expression in the inner ear, which is toxic and damages hearing. Accordingly, in some embodiments, the isolated nucleic acid described herein is capable of expressing the GJB2 protein in inner ear cells that normally express the GJB2 gene (e.g., connective tissue cells of the cochlea and supporting cells of the organ of Corti and nearby regions), but not in the cell that do not normally express GJB2 (e.g., hair cells and spiral ganglion neurons).

In some aspects, the present disclosure provides an isolated nucleic acid comprising an expression cassette, wherein the expression cassette comprises a gap junction beta 2 (GJB2) gene regulatory element (GRE), and a nucleotide sequence encoding a GJB2 protein.

In some embodiments, the GJB2 protein is a human GJB2 protein. In some embodiments, the GJB2 protein comprises an amino acid sequence at least 80% identical to SEQ ID NO: 1. In some embodiments, the nucleotide sequence encoding a human GJB2 protein comprises a nucleotide sequence at least 80% identical to SEQ ID NO: 2.

In some embodiments, the expression cassette further comprises a promoter operably linked to the nucleotide sequence encoding a GJB2 protein. In some embodiments, the promoter is a human GJB2 promoter. In some embodiments, the promoter comprises 500 nucleotides of a human GJB2 promoter. In some embodiments, the promoter comprises a nucleic acid sequence at least 80% identical to SEQ ID NO: 5. In some embodiments, the promoter comprises a nucleic acid sequence at least 80% identical to SEQ ID NO: 102. In some embodiments, the promoter comprises a nucleic acid sequence 100% identical to SEQ ID NO: 102.

In some embodiments, the promoter is a human GJB2 basal promoter. In some embodiments, the human GJB2 basal promoter comprises a nucleic acid sequence at least 80% identical to SEQ ID NO: 47.

In some embodiments, the expression cassette comprises a nucleotide sequence encoding a 5′ UTR. In some embodiments, the 5′ UTR is positioned between the promoter and the nucleotide sequence encoding the GJB2 protein. In some embodiments, the 5′ UTR comprises about 300 nucleotides of a human GJB2 gene 5′ UTR. In some embodiments, the promoter and the 5′ UTR comprises a nucleotide sequence at least 80% identical to SEQ ID NO: 30.

In some embodiments, the GJB2 gene regulatory element comprises an enhancer. In some embodiments, the enhancer is positioned 5′ to the promoter. In some embodiments, the enhancer is normally present within approximately 200 kb upstream or downstream of a GJB2 gene. In some embodiments, the enhancer is normally present within approximately 95 kb of a GJB2 gene. In some embodiments, the GJB2 GRE comprises one or more enhancers. In some embodiments, the one or more enhancers are the same enhancers or different enhancers. In some embodiments, the enhancer comprises a nucleotide sequence at least 80% identical to nucleotide sequence or a fragment thereof as set forth in any one of SEQ ID NOs: 6 to 29. In some embodiments, the enhancer comprises a nucleotide sequence at least 80% identical to a GJB2 enhancer as set forth in any of SEQ ID NOs: 37-46 and 55-60. In some embodiments, the enhancer comprises a nucleotide sequence at least 80% identical to SEQ ID NO: 42.

In some aspects, the present disclosure also provides an isolated nucleic acid comprising an expression cassette, wherein the expression cassette comprises a Gap Junction beta 2 (GJB2) promoter, and a nucleotide sequence encoding a GJB2 protein.

In some embodiments, the GJB2 promoter comprises a nucleic acid sequence at least 80% identical to SEQ ID NO: 102. In some embodiments, the GJB2 promoter comprises a nucleic acid sequence 100% identical to SEQ ID NO: 102.

In some embodiments, the expression cassette further comprises a 5′ UTR. In some embodiments, the 5′ UTR comprises: a first nucleic acid sequence at least 80% identical to SEQ ID NO: 103; and/or a second nucleic acid sequence at least 80% identical to SEQ ID NO: 104. In some embodiments, the expression cassette further comprises a 5′ UTR. In some embodiments, the 5′ UTR comprises: a first nucleic acid sequence 100% identical to SEQ ID NO: 103; and/or a second nucleic acid sequence 100% identical to SEQ ID NO: 104.

In some embodiments, the isolated nucleic acid comprises a nucleic acid sequence at least 80% identical to SEQ ID NO: 105. In some embodiments, the isolated nucleic acid comprises a nucleic acid sequence 100% identical to SEQ ID NO: 105.

In some embodiments, the isolated nucleic acid is capable of expressing GJB2 in cells that normally express the GJB2 gene. In some embodiments, the isolated nucleic acid is capable of expressing GJB2 in cochlear connective tissue cells and supporting cells of the organ of Corti. In some embodiments, the supporting cell of the organ of Corti are pillar cells, Deiter cells, Hensen's cells, Claudius cells, inner phalangeal cells, and border cells. In some embodiments, the cochlear connective tissue cells are strial intermediate cells, fibrocytes of the lateral wall and suprastrial zone, basal cells of the stria vascularis, fibrocytes in the spiral ligament, fibrocytes in the spiral limbus, mesenchymal cells lining the bony otic capsule facing the scala vestibuli, and supralimbal dark cells.

In some embodiments, the expression cassette is flanked by two adeno-associated virus inverted terminal repeats (ITRs). In some embodiments, the AAV ITR is from a serotype selected from the group consisting of AAV1 ITR, AAV2 ITR, AAV3 ITR, AAV4 ITR, AAV5 ITR, and AAV6 ITR. In some embodiments, the AAV ITR is AAV2 ITR.

In some embodiments, the expression cassette comprises: a 5′ ITR having a nucleotide sequence at least 80% identical to SEQ ID NO: 106; and/or a 3′ ITR having a nucleotide sequence at least 80% identical to SEQ ID NO: 107. In some embodiments, the expression cassette comprises: a 5′ ITR having a nucleotide sequence 100% identical to SEQ ID NO: 106; and/or a 3′ ITR having a nucleotide sequence 100% identical to SEQ ID NO: 107.

In some embodiments, the expression cassette further comprises a Woodchuck Hepatitis Virus (WHP) Posttranscriptional Regulatory Element (WPRE) 3′ to the nucleotide sequence encoding the GJB2 protein.

In some embodiments, the WPRE comprises a nucleotide sequence at least 80% identical to SEQ ID NO: 108. In some embodiments, the WPRE comprises a nucleotide sequence 100% identical to SEQ ID NO: 108.

In some embodiments, the expression cassette further comprises a nucleotide sequence encoding a 3′ UTR located 5′ of the WPRE. In some embodiments, the 3′ UTR is a GJB2 exon 2 3′ UTR. In some embodiments, the GJB2 exon 2 3′ UTR comprises a nucleotide sequence at least 80% identical to SEQ ID NO: 32.

In some embodiments, the expression cassette further comprises one or more miRNA binding site positioned in the 3′ UTR. In some embodiments, the miRNA binding site is a neuron-associated miRNA binding site. In some embodiments, the neuron-associated miRNA is selected from: miR-124, miR-127, miR-129, miR-129*, miR-136, miR-136*, miR-137, miR-154, miR-300-3p, miR-323, miR-329, miR-341, miR-369-5p, miR-376a, miR-376b-3p, miR-376c, miR-379, miR-382, miR-382*, miR-410, miR-411, miR-433, miR-434, miR-495, miR-541, miR-543*, miR-551b, miR-143, miR-449a, miR-219-2-3p, miR-126, miR-126*, miR-141, miR-142-3p, miR-142-5p, miR-146a, miR-150, miR-200c, and miR-223. In some embodiments, the neuron-associated miRNA is miR-124. In some embodiments, the miRNA binding site is a cochlear hair cell-associated miRNA binding site. In some embodiments, the cochlear hair cell-associated miRNA binding site is selected from: miR-124, miR-96, miR-182, and miR-183.

In some embodiments, the expression cassette further comprises a poly A signal. In some embodiments, the poly A signal is a bovine growth hormone poly A signal.

In some embodiments, the poly A signal comprises a nucleotide sequence at least 80% identical to SEQ ID NO: 109. In some embodiments, the poly A signal comprises a nucleotide sequence 100% identical to SEQ ID NO: 109.

In some aspects, the present disclosure also provides an isolated nucleic acid comprising a nucleotide sequence 100% identical to SEQ ID NO: 110 or 111. In some aspects, the present disclosure also provides an isolated nucleic acid comprising a nucleotide sequence at least 80% identical to SEQ ID NO: 110 or 111.

In some aspects, the present disclosure also provides a vector comprising the isolated nucleic acid as described herein. In some embodiments, the vector is a plasmid or a viral vector. In some embodiments, the viral vector is an AAV vector.

In some aspects, the present disclosure also provides a vector comprising from 5′ to 3′: (a) an AAV 5′ ITR; (b) a GJB2 promoter, or a basal GJB2 promoter sequence thereof; (c) a GJB2 5′ UTR (e.g., a GJB2 exon 1 5′ UTR); (d) a nucleotide sequence encoding a GJB2 protein; (e) a GJB2 3′ UTR (e.g., a GJB2 exon 2 3′ UTR), optionally the GJB2 3′ UTR comprises one or more miR-124 binding site; (f) a bovine growth hormone poly A signal; and (g) an AAV 3′ ITR.

In some aspects, the present disclosure also provides a vector comprising from 5′ to 3′: (a) an AAV 5′ ITR; (b) a GJB2 enhancer; (c) a GJB2 promoter, or a basal GJB2 promoter sequence thereof; (d) a GJB2 5′ UTR (e.g., a GJB2 exon 1 5′ UTR); (e) a nucleotide sequence encoding a GJB2 protein; (f) a GJB2 3′ UTR (e.g., a GJB2 exon 2 3′ UTR), optionally the GJB2 3′ UTR comprises one or more miR-124 binding site; (g) a bovine growth hormone poly A signal; and (h) an AAV 3′ ITR.

In some embodiments, the vector comprises a nucleotide sequence at least 80% identical to any one of SEQ ID NOs: 36, 48-62 and 61-83. In some embodiments, the vector is an AAV vector. In some embodiments, the vector is capable of expressing a GJB2 gene in cells that normally express GJB2.

In some aspects, the present disclosure also provides a recombinant adeno-associated virus (rAAV) comprising: (i) a capsid protein; and (ii) the isolated nucleic acid described herein.

In some aspects, the present disclosure also provides a recombinant adeno-associated virus (rAAV) comprising: (i) a capsid protein; and (ii) an isolated nucleic acid comprising: (a) an AAV 5′ ITR (e.g., a GJB2 exon 1 5′ UTR); (b) a GJB2 promoter, or a basal GJB2 promoter sequence thereof; (c) a GJB2 5′ UTR (e.g., a GJB2 exon 2 3′ UTR), optionally the GJB2 exon 2 3′ UTR comprises one or more miR-124 binding site; (d) a nucleotide sequence encoding a GJB2 protein; (e) a GJB2 3′ UTR; (f) a bovine growth hormone poly A signal; and (g) an AAV 3′ ITR.

In some aspects, the present disclosure also provides a recombinant adeno-associated virus (rAAV) comprising: (i) a capsid protein; and (ii) an isolated nucleic acid comprising: (a) an AAV 5′ ITR; (b) a GJB2 enhancer; (c) a GJB2 promoter, or a basal GJB2 promoter sequence thereof; (d) a GJB2 5′ UTR (e.g., a GJB2 exon 1 5′ UTR); (e) a nucleotide sequence encoding a GJB2 protein; (f) a GJB2 3′ UTR (e.g., a GJB2 exon 2 3′ UTR), optionally the GJB2 exon 2 3′ UTR comprises one or more miR-124 binding site; (g) a bovine growth hormone poly A signal; and (h) an AAV 3′ ITR.

In some embodiments, the rAAV has tropism for a subset of cochlea cells that normally express the GJB2 gene. In some embodiments, the rAAV has tropism for cells of the inner ear.

In some embodiments, the capsid protein is an AAV1 capsid protein, an AAV2 capsid protein, an AAV5 capsid protein, an AAV7 capsid protein, an AAV8 capsid protein, an AAV9 capsid protein, an AAV-S capsid protein, or a variant thereof. In some embodiments, the AAV capsid is AAV9.PHP.B, AAV9.PHP.eB, or AAV-S. In some embodiments, the AAV capsid protein is AAV-S.

In some aspects, the present disclosure provides a host cell comprising the isolated nucleic acid, the vector, or the rAAV as described herein.

In some aspects, the present disclosure provides a pharmaceutical composition comprising the isolated nucleic acid, the vector, the rAAV, or the host cell as described herein. In some embodiments, the pharmaceutical composition further comprises a pharmaceutically acceptable carrier.

In some aspects, the present disclosure provides a method for specifically expressing GJB2 in cells that normally expresses the GJB2 gene in a subject, the method comprising administering to the subject an effective amount of the isolated nucleic acid, the vector, the rAAV, the host cell, or the pharmaceutical composition as described herein.

In some aspects, the present disclosure provides a method for treating Non-syndromic Hearing Loss and Deafness (DFNB1) in a subject, the method comprising administering to the subject an effective amount of the isolated nucleic acid, the vector, the rAAV, the host cell, or the pharmaceutical composition as described herein.

A method for treating a GJB2-associated disease in a subject in need thereof, the method comprising administering to the subject an effective amount of the isolated nucleic acid, the vector, the rAAV, the host cell, or the pharmaceutical composition as described herein.

In some embodiments, the subject is a mammal. In some embodiments, the mammal is a human. In some embodiments, the mammal is a non-human mammal. In some embodiments, the non-human mammal is mouse, rat, or non-human primate.

In some embodiments, the hearing loss is associated with a mutation in the GJB2 gene. In some embodiments, the mutation in the GJB2 gene is a point mutation, a missense mutation, a nonsense mutation, a splice-altering mutation, a synonymous mutation, a deletion, an insertion, or a combination thereof. In some embodiments, the subject is human; and the mutation is a mutation listed in Table 2 (below) or a combination thereof. In some embodiments, the mutation is NM_004004.6 c.101T>C (GRCh37/hg19 Chr13:20763620A>G) or c.35delG (GRCh37/hg19 chr13:20763685AC>A).

In some embodiments, the administration results in expression of GJB2 protein in the cochlea connective tissue cells and supporting cells of the organ of Corti and nearby regions. In some embodiments, the supporting cell of the organ of Corti are pillar cells, Deiters' cells, Hensen's cells, Claudius cells, inner phalangeal cells, and border cells. In some embodiments, the connective tissue cells are strial intermediate cells, fibrocytes of the lateral wall and suprastrial zone, basal cells of the stria vascularis, fibrocytes in the spiral ligament, fibrocytes in the spiral limbus, mesenchymal cells lining the bony otic capsule facing the scala vestibuli, and supralimbal dark cells.

In some embodiments, the administration is via injection. In some embodiments, the injection is through round window membrane of the cochlea, into the scala media of the cochlea, into the scala tympani of the cochlea, into the scala vestibuli of the cochlea, into a semicircular canal of the inner ear, or into the saccule or the utricle of the inner ear.

The details of one or more embodiments of the invention are set forth in the description below. Other features or advantages of the present invention will be apparent from the following drawing and detailed description of certain embodiments and also from the appended claims.

### BRIEF DESCRIPTION OF THE DRAWINGS

The accompanying drawings, which are incorporated in and constitute a part of this specification, illustrate certain embodiments, and together with the written description, serve to provide non-limiting examples of certain aspects of the compositions and methods disclosed herein.

FIGS. 1A-1C show the structure and expression distribution of GJB2, and how loss of GJB2 expression affects the patients. FIG. 1A shows the structure of the GJB2 hemichannel. Six subunits of GJB2 protein, each with four trans-membrane helices, assemble in the plane of the membrane to form a large central pore. GJB2 hemichannels from adjacent cells join to create a channel from the cytoplasm of one cell to the cytoplasm of the other. Gap junctions are formed by hundreds or thousands of channels packed in a junctional plaque. FIGS. 1B-1C show the network of fibrocytes and epithelial cells in which GJB2 is expressed (FIG. 1B), and the inner and outer hair cells, in which GJB2 is not expressed (FIG. 1C). FIG. 1D shows that many patients carrying GJB2 mutation(s) who have some residual hearing at birth show further hearing loss over the next 3-6 years. A window for treatment is present for 1-5 years after birth. with ˜10,000 affected children in the United State aged 0-5 possibly treatable.

FIGS. 2A-2B show the delivery of viral vector to the cochlea by direct injection through the round window membrane (RWM) and the deleterious effect of promiscuous expression of Gjb2 to the hearing of injected mice. FIG. 2A is a cartoon illustrating the round window membrane (RWM) injection. FIG. 2B shows that promiscuous expression of Gjb2 in the inner ear damaged hearing in wild-type mice.

FIGS. 3A-3N show the identification of cis-regulatory elements (e.g., enhancers) that are critical for GJB2 expression in the subset of cochlea cells that naturally express the GJB2 gene. FIGS. 3A-3B show that certain patients with GJB2-associated deafness have upstream deletions occurring in trans with GJB2 coding sequence mutations, which suggests that some patients carry mutation(s) in the cis-regulatory element, and the region next to the CRYL1 gene is of particular importance for identification of such cis-regulatory elements. FIG. 3C (top) shows the identification of gene regulatory elements (GREs), in UCSC Genome Browser views of ATAC-Seq from mouse cochlea at developmental stages P2, P5 and P8, over ˜300 kb in the region of the mouse Gjb2 gene. Shaded regions mark regions containing putative GREs. X-axis is the genomic region on chr14 in the mouse genome. Y-axis is the number of reads from the ATAC-Seq that align to a specific region in the genome. Light shading denotes regions of open chromatin, which are the hallmarks of transcriptionally active regions that are enriched for read pile up, suggesting higher activity in these regions. Regions A and B mark the transcriptionally active sequences within mouse Gjb2 itself. Regions C-M are regions that are transcriptionally active around Gjb2 that might be part of a cis-regulatory network. FIG. 3C (bottom) shows transcriptionally active regions in and around the light-shaded regions that have been tested as specific GREs (dark highlight). Note that the GREs were initially identified in mouse. Human GJB2 GREs were identified in silico by modeling the mouse GREs. Human GJB2 GREs were tested in subsequent experiments. FIGS. 3D-3E show various vector designs with or without incorporation of GJB2 promoter and/or enhancers. These vectors were tested in mouse inner ear. The C15 vector, which is the GJB2 enhancer vector, concatenates 500 bp of the human GJB2 promoter, the human GJB2 5′ UTR followed by a coding sequence for GFP and human GJB2 3′ UTR, and three human GJB2 enhancers that match mouse sequences identified by ATAC-seq. Vectors c20-23 were constructed to test the toxicity of promiscuous expression of Gjb2 in mouse. Vector c20 was lethal at doses over 2×10⁹ genomic copies. FIG. 3F shows a segment of the mouse cochlea, from the lateral wall (top) to the interdental cells (bottom). Cells transduced with the AAV9-PHP.B-C15 vector and expressing the GFP marker gene under Gjb2 enhancers are shown in the left panel. Cells normally expressing GJB2 are shown in the middle panel. In the right panel, IHCs and OHCs (indicated) are also identified by labeling actin with fluorescent phalloidin. The expression pattern of GFP, which was driven by the c15 construct, is consistent with native Gjb2 expression reported in Kikuchi et al., 1995 using the same antibody against GJB2. Notably, c15 does not drive GFP expression in hair cells. FIG. 3G shows the expression of Gjb2 in inner hair cells driven by construct c20. 3D reconstruction of the organ of Corti in an uninjected mouse cochlea, with outer hair cells and inner hair cells is shown in the top panel. GJB2-containing gap junctions in supporting cells were labeled with an antibody to GJB2 protein. Hair cells do not make gap junctions. Vector c20, with a promiscuous promoter, drives GJB2 expression in inner hair cells and other cell types (see bottom panel). FIG. 3H shows that promiscuous Gjb2 expression damages hearing in wild-type mice, but targeted expression rescues hearing in Gjb2 knockout mice. However, a C70 construct, which includes GJB2 promoter/enhancer based on preliminary results from the ATAC-Seq, was capable of rescuing hearing by 15-20 dB, and did not damage hearing in the wild-type. FIGS. 3I-3L shows the map of the c70 vector plasmid encoding mouse GJB2 or human GJB2 with or without an HA tag. FIG. 3M shows schematics of vector c.70 encoding mouse GJB2 or human GJB2 with or without the HA tag. FIG. 3N shows additional vectors that were created and tested.

FIG. 4 shows that AAV-S encoding eGFP with a CBA promoter efficiently transduces hair cells, supporting cells, and cells of the lateral wall, in both neonatal mouse and juvenile NHP cochlea.

FIGS. 5A-5V show vector maps of the AAV vectors including the identified GJB2 GREs 1, 2, 3, 4, 5, 7, 8, and 9, respectively. The vectors include, from 5′ to 3′, a 5′ ITR, a human GJB2 GRE, a GJB2 basal promoter, a GJB2 exon 1 5′ UTR, a nucleotide sequence encoding an eGFR, and GJB2 exon 2 3′ UTR. FIG. 5A shows vector c.81.1, which includes human GJB2 GRE1, and encodes human GJB2; FIG. 5B shows vector c.81.1, which includes human GJB2 GRE1, and encodes mouse GJB2; FIG. 5C shows vector c.81.2, which includes human GJB2 GRE2, and encodes eGFP; FIG. 5D shows vector c.81.2, which includes human GJB2 GRE2, and encodes human GJB2; FIG. 5E shows vector c.81.2, which includes human GJB2 GRE2, and encodes mouse GJB2; FIG. 5F shows vector c.81.3, which includes human GJB2 GRE3, and encodes eGFP; FIG. 5G shows vector c.81.3, which includes human GJB2 GRE3, and encodes human GJB2; FIG. 5H shows vector c.81.3, which includes human GJB2 GRE3, and encodes mouse GJB2; FIG. 5I shows vector c.81.4, which includes human GJB2 GRE4, and encodes human GJB2; FIG. 5J shows vector c.81.4, which includes human GJB2 GRE4, and encodes mouse GJB2; FIG. 5K shows vector c.81.5, which includes human GJB2 GRE5, and encodes eGFP; FIG. 5L shows vector c.81.5, which includes human GJB2 GRE5, and encodes human GJB2; FIG. 5M shows vector c.81.5, which includes human GJB2 GRE5, and encodes mouse GJB2; FIG. 5N shows vector c.81.7, which includes human GJB2 GRE7, and encodes eGFP; FIG. 5O shows vector c.81.7, which includes human GJB2 GRE7, and encodes human GJB2; FIG. 5P shows vector c.81.7, which includes human GJB2 GRE7, and encodes mouse GJB2; FIG. 5Q shows vector c.81.8, which includes human GJB2 GRE8, and encodes human GJB2; FIG. 5R shows vector c.81.8, which includes human GJB2 GRE8, and encodes mouse GJB2; FIG. 5S shows vector c.81.9, which includes human GJB2 GRE9, and encodes eGFP; FIG. 5T shows vector c.81.9, which includes human GJB2 GRE9, and encodes human GJB2; FIG. 5U shows vector c.81.9, which includes human GJB2 GRE9, and encodes mouse GJB2. FIG. 5V shows schematics of c81.2, c81.3, c81.5, c81.7 and c81.9 encoding eGFP, mouse GJB2 and human GJB2 as described above.

FIGS. 6A-6D show GFP expression by vector c81.5 in the cells of the organ of Corti FIG. 6A shows a fluorescent image of GFP expressing cells, including a variety of supporting cells in, and medial to, the organ of Corti. FIG. 6B shows antibody label of endogenous GJB2 in the region of the organ of Corti. Gjb2 expression largely overlapped that of exogenous GFP. FIG. 6C is an overlay of FIGS. 6A and 6B, with a third staining of actin, which revealed stereocilia of hair cells. No GFP was expressed in the hair cells. FIG. 6D shows a frozen section immunofluorescence image of GFP and a protein marker for hair cells, MYO7A. GFP was expressed in a variety of supporting cells in the organ of Corti, but did not overlap with MYO7A expression, which was expressed in hair cells.

FIGS. 7A-7E show GFP expression pattern by vector 81.5 in the lateral wall of the cochlea. FIG. 7A shows GFP expression in cells including fibrocytes of the lateral wall. FIG. 7B shows an antibody labeling of endogenous Gjb2 in the region of the lateral wall. GJB2 expression largely overlaps that of exogenous GFP. FIG. 7C is an overlay image of FIGS. 7A and 7B. Note that GFP was expressed in the cells expressing Gjb2. FIGS. 7D-7E show frozen section immunofluorescences of GFP (FIG. 7D) and GJB2 in supporting cells of the organ of Corti and fibrocytes of the lateral wall (FIG. 7E).

The accompanying drawings, which are incorporated in and constitute a part of this specification, illustrate certain embodiments, and together with the written description, serve to provide non-limiting examples of certain aspects of the compositions and methods disclosed herein.

### DETAILED DESCRIPTION

The present disclosure, at least in part, relates to an isolated nucleic acid comprising an expression cassette, wherein the expression cassette comprises a gap junction beta 2 (GJB2) gene regulatory element (GRE), and a nucleotide sequence encoding a GJB2 protein. In some embodiments, the expression cassette further comprises a promoter (e.g., GJB2 promoter). In some embodiments, the expression cassette is flanked by two adeno-associated virus (AAV) inverted terminal repeats (ITRs). The presence of native GJB2 regulatory elements (GREs) in the isolated nucleic acid prevents promiscuous GJB2 gene expression in the inner ear, which is toxic and damages hearing. Accordingly, in some embodiments, the isolated nucleic acid described herein is capable of expressing the GJB2 protein in inner ear cells that normally express the GJB2 gene (e.g., connective tissue cells of the cochlea and supporting cells of the organ of Corti and nearby regions), but not in the cell that do not normally express GJB2 gene (e.g., hair cells and spiral ganglion neurons).

### I. Isolated Nucleic Acid

In some aspects, the present disclosure relates to compositions and methods for treating certain autosomal recessive genetic diseases, for example, non-syndromic hearing loss (DFNB1). DFNB1 is caused by mutations in the GJB2 gene. The GJB2 gene encodes the GJB2 protein, also known as connexin 26. Connexin 26 is a member of the connexin protein family. GJB2 protein forms channels in clusters called gap junctions, which allow communication between neighboring cells, including cells in the inner ear. Mutations in the GJB2 gene eliminate or change the structure of gap junctions and affect the function or survival of cells that are needed for hearing. Gene replacement therapy (e.g., gene therapy by recombinant adeno-associated virus (rAAVs)) is attractive due to the small size of the GJB2 gene coding sequence (less than 700 bp). However, restoration of GJB2 expression in the inner ear using the currently available gene therapy does not lead to the restoration of hearing.

Accordingly, the present disclosure is based, in part, on the surprising discovery that successful GJB2 gene therapy requires GJB2 expression in cells that normally express the GJB2 protein (e.g., connective tissue cells of the cochlea and supporting cells of the organ of Corti and nearby regions) and not in other cells (e.g., hair cells and spiral ganglion neurons). Excluding sensory cells, most cells in the cochlea are connected via gap junctions, and these gap junctions appear to play a critical role in cochlear function. GJB2 protein occurs in gap junctions connecting most cell classes in the cochlea. There are two independent systems of cells, which are defined by interconnecting gap junctions. The first system, the epithelial cell gap junction system, is mainly composed of all organs of Corti supporting cells (e.g., epithelial cells of the inner and outer sulcus, and interdental cells), and also includes interdental cells in the spiral limbus and root cells within the spiral ligament. In the inner ear, the sensory region of the cochlea, termed the organ of Corti, includes one row of inner hair cells (IHC) and three to four rows of outer hair cells (OHC) that are surrounded by various supporting cells. The supporting cells play crucial roles in the development, function, and maintenance of inner ear sensory epithelia. Unlike hair cells, which contact only the lumenal surface of the epithelium, supporting cells span the entire depth of the epithelium, from the basal lamina to the lumen. Supporting cells are linked to each other and to hair cells by tight and adherens junctions; they communicate directly with other supporting cells by gap junctions (e.g., Wan et al., Inner ear supporting cells: Rethinking the silent majority, Semin Cell Dev Biol. 2013 May; 24(5): 448-459). Non-limiting examples of supporting cells for the organ of Corti include pillar cells, Deiters' cells, Hensen's cells, Claudius cells, inner phalangeal cells, and border cells. The second system, the connective tissue cell gap junction system, includes strial intermediate cells, fibrocytes of the lateral wall and suprastrial zone, basal cells of the stria vascularis, fibrocytes in the spiral ligament, fibrocytes in the spiral limbus, mesenchymal cells lining the bony otic capsule facing the scala vestibuli, and supralimbal dark cells. In some embodiments, in the cochlea, GJB2 is normally expressed in supporting cells of the organ of Corti and nearby regions (e.g., pillar cells, Deiters' cells, Hensen's cells, Claudius cells, inner phalangeal cells; and border cells), and the connective tissue system comprising strial intermediate cells, fibrocytes of the lateral wall and suprastrial zone, basal cells of the stria vascularis, fibrocytes in the spiral ligament, fibrocytes in the spiral limbus, mesenchymal cells lining the bony otic capsule facing the scala vestibuli, and supralimbal dark cells (See, e.g., Kikuchi et al. (1995) Gap junctions in the rat cochlea: immunohistochemical and ultrastructural analysis. Anat Embryol (Berl) 191:101-118; and Kikuchi et al., Gap junction systems in the mammalian cochlea, Brain Res Brain Res Rev. 2000 April;32(1):163-6. doi: 10.1016/s0165-0173(99)00076-4.).

GJB2 expression is critical for cochlear function. For example, the K⁺ that enters hair cells through transduction channels and leaves through basal K⁺ channels is shuttled away from the organ of Corti by the epithelial system and conveyed by the cytoplasmic system to the stria, where it is pumped back into endolymph. Further, GJB2 plays a role in the development of the cochlea, as mice lacking GJB2 protein in the inner ear have reduced endocochlear potential and profound apoptotic loss of hair cells and supporting cells by postnatal day 30 (P30), even though hair cells do not express Gjb2 (Cohen-Salmon et al., 2002; Wang et al., 2009; Sun et al., 2009; Crispino et al., 2011; Johnson et al., 2017). If Gjb2 is deleted after P6, the phenotype is much milder (Chang et al., 2015). However there remains a long-term requirement for GJB2 protein: hair cell loss occurs after months even with deletion as late as P14 (Ma et al., 2020). Not wishing to be bound by any particular theory, GJB2's function in shuttling K⁺ may be related to its role in the development of the cochlea: If K⁺ is not carried away from hair cells by a gap junction network, K⁺ accumulation could depolarize hair cells, leading to Ca²⁺ influx and eventual cell death. The gap junction network may also be required to transport glucose and nutrients from blood vessels to the sensory epithelium, and its absence could lead to cell death.

In some embodiments, the present disclosure provides an isolated nucleic acid comprising two adeno-associated virus (AAV) inverted terminal repeats (ITRs) flanking an expression cassette, wherein the expression cassette comprises a promoter (e.g., a human GJB2 promoter) operably linked to a nucleotide sequence encoding a GJB2 gene regulatory element (GRE), and a nucleotide sequence encoding a gap junction beta 2 (GJB2) protein. Incorporation of the native GJB2 gene regulatory element and/or tissue/cell-specific promoter in the isolated nucleic acid facilitates the expression of the GJB2 gene in cells that normally express it (e.g., connective tissue cells of the cochlea including fibrocytes and supporting cells of the organ of Corti and nearby regions). An expression cassette, as used herein, refers to component of vector DNA comprising a protein coding sequence to be expressed by a cell having the vector and its regulatory sequences. Once delivered to the target cell, the expression cassette directs the cell's machinery to make RNA and/or protein(s) (e.g., GJB2 protein).

A “nucleic acid” sequence refers to a DNA or RNA sequence. In some embodiments, proteins and nucleic acids of the disclosure are isolated. As used herein, the term “isolated” means artificially produced. As used herein with respect to nucleic acids, the term “isolated” means: (i) amplified in vitro by, for example, the polymerase chain reaction (PCR); (ii) recombinantly produced by cloning; (iii) purified, for example, by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

In some embodiments, the GJB2 protein is a human GJB2 protein. In some embodiments, the human GJB2 protein comprises an amino acid sequence at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1.

An exemplary human GJB2 protein sequence is set forth in SEQ ID NO: 1:

MDWGTLQTILGGVNKHSTSIGKIWLTVLFIFRIMILVVAA
KEVWGDEQADFVCNTLQPGCKNVCYDHYFPISHIRLWALQ
LIFVSTPALLVAMHVAYRRHEKRKFIKGEIKSEFKDIEEI
KTQKVRIEGSLWWTYTSSIFFRVIFEAAFMYVFYVMYDGF
SMQRLVKCNAWPCPNTVDCFVSRPTEKTVFTVFMIAVSGI
CILLNVTELCYLLIRYCSGKSKKPV

In some embodiments, the expression cassette of the isolated nucleic acid encodes a human GJB2 protein having the amino acid sequence as set forth in SEQ ID NO: 1. In some embodiments, the nucleotide sequence encoding a human GJB2 protein comprises a nucleotide sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 2.

An exemplary nucleotide sequence encoding a human GJB2 protein is set forth in SEQ ID NO: 2:

ATGGATTGGGGCACGCTGCAGACGATCCTGGGGGGTGTGA
ACAAACACTCCACCAGCATTGGAAAGATCTGGCTCACCGT
CCTCTTCATTTTTCGCATTATGATCCTCGTTGTGGCTGCA
AAGGAGGTGTGGGGAGATGAGCAGGCCGACTTTGTCTGCA
ACACCCTGCAGCCAGGCTGCAAGAACGTGTGCTACGATCA
CTACTTCCCCATCTCCCACATCCGGCTATGGGCCCTGCAG
CTGATCTTCGTGTCCACGCCAGCGCTCCTAGTGGCCATGC
ACGTGGCCTACCGGAGACATGAGAAGAAGAGGAAGTTCAT
CAAGGGGGAGATAAAGAGTGAATTTAAGGACATCGAGGAG
ATCAAAACCCAGAAGGTCCGCATCGAAGGCTCCCTGTGGT
GGACCTACACAAGCAGCATCTTCTTCCGGGTCATCTTCGA
AGCCGCCTTCATGTACGTCTTCTATGTCATGTACGACGGC
TTCTCCATGCAGCGGCTGGTGAAGTGCAACGCCTGGCCTT
GTCCCAACACTGTGGACTGCTTTGTGTCCCGGCCCACGGA
GAAGACTGTCTTCACAGTGTTCATGATTGCAGTGTCTGGA
ATTTGCATCCTGCTGAATGTCACTGAATTGTGTTATTTGC
TAATTAGATATTGTTCTGGGAAGTCAAAAAAGCCAGTT

In some embodiments, the GJB2 protein is a mouse GJB2 protein. In some embodiments, the mouse GJB2 protein comprises an amino acid sequence at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 3.

An exemplary mouse GJB2 protein sequence is set forth in SEQ ID NO: 3:

MDWGTLQSILGGVNKHSTSIGKIWLTVLFIFRIMILVVAA
KEVWGDEQADFVCNTLQPGCKNVCYDHHFPISHIRLWALQ
LIMVSTPALLVAMHVAYRRHEKKRKFMKGEIKNEFKDIEE
IKTQKVRIEGSLWWTYTTSIFFRVIFEAVFMYVFYIMYNG
FFMQRLVKCNAWPCPNTVDCFISRPTEKTVFTVFMISVSG
ICILLNITELCYLFVRYCSGKSKRPV

In some embodiments, the isolated nucleic acid comprises a nucleotide sequence encoding a mouse GJB2 protein having an amino acid sequence as set forth in SEQ ID NO: 3. In some embodiments, the nucleotide sequence encoding a mouse GJB2 protein comprises a nucleotide sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 4.

An exemplary nucleotide sequence encoding a mouse GJB2 protein is set forth in SEQ ID NO: 4:

ATGGATTGGGGCACACTCCAGAGCATCCTCGGGGGTGTCA
ACAAACACTCCACCAGCATTGGAAAGATCTGGCTCACGGT
CCTCTTCATCTTCCGCATCATGATCCTCGTGGTGGCTGCA
AAGGAGGTGTGGGGAGATGAGCAAGCCGATTTTGTCTGCA
ACACGCTCCAGCCTGGCTGCAAGAATGTATGCTACGACCA
CCACTTCCCCATCTCTCACATCCGGCTCTGGGCTCTGCAG
CTGATCATGGTGTCCACGCCAGCCCTCCTGGTAGCTATGC
ATGTGGCCTACCGGAGACATGAAAAGAAACGGAAGTTCAT
GAAGGGAGAGATAAAGAACGAGTTTAAGGACATCGAAGAG
ATCAAAACCCAGAAGGTCCGTATCGAAGGGTCCCTGTGGT
GGACCTACACCACCAGCATCTTCTTCCGGGTCATCTTTGA
AGCCGTCTTCATGTACGTCTTTTACATCATGTACAATGGC
TTCTTCATGCAACGTCTGGTGAAATGCAACGCTTGGCCCT
GCCCCAATACAGTGGACTGCTTCATTTCCAGGCCCACAGA
AAAGACTGTCTTCACCGTGTTTATGATTTCTGTGTCTGGA
ATTTGCATTCTGCTAAATATCACAGAGCTGTGCTATTTGT
TCGTTAGGTATTGCTCAGGAAAGTCCAAAAGACCAGTC

In some embodiments, the nucleotide sequence encoding the GJB2 protein is codon optimized for expression in a host (e.g., a human). “Codon optimization” as described herein, refers to the design process of altering codons to codons known to increase maximum protein expression efficiency in a desired cell. In some alternatives, codon optimization is described, wherein codon optimization can be performed by using algorithms that are known to those skilled in the art to create synthetic genetic transcripts optimized for high protein yield. Programs containing algorithms for codon optimization are known to those skilled in the art. Programs can include, for example, OptimumGene™, GeneGPS® algorithms, etc. Additionally, synthetic codon optimized sequences can be obtained commercially, for example from Integrated DNA Technologies and other commercially available DNA sequencing services.

As used herein, the term “sequence identity” refers to the percentage of amino acid (or nucleic acid) residues of a candidate sequence that are identical to the amino acid (or nucleic acid) residues of a reference sequence, e.g., GJB2 protein disclosed herein and its coding sequences, after aligning the sequences and introducing gaps, if necessary, to achieve the maximum percent identity (e.g., gaps can be introduced in one or both of the candidate and reference sequences for optimal alignment and non-homologous sequences can be disregarded for comparison purposes). Alteration of the amino acid sequence or nucleic acid coding sequences can be obtained by deletion, addition, or substitution of residues of the reference sequence. Alignment for purposes of determining percent identity can be achieved in various ways that are within the skill of one in the art, for instance, using publicly available computer software, such as BLAST, BLAST-2, BLAST-P, BLAST-N, BLAST-X, WU-BLAST-2, ALIGN, ALIGN-2, CLUSTAL, or Megalign (DNASTAR) software. Those skilled in the art can determine appropriate parameters for measuring alignment, including any algorithms needed to achieve maximal alignment over the full length of the sequences being compared. For instance, the percent amino acid (or nucleic acid) sequence identity of a given candidate sequence to, with, or against a given reference sequence (which can alternatively be phrased as a given candidate sequence that has or includes a certain percent amino acid (or nucleic acid) sequence identity to, with, or against a given reference sequence) is calculated as follows:

100×(fraction of A/B)

where A is the number of amino acid (or nucleic acid) residues scored as identical in the alignment of the candidate sequence and the reference sequence, and where B is the total number of amino acid (or nucleic acid) residues in the reference sequence. In particular, a reference sequence aligned for comparison with a candidate sequence can show that the candidate sequence exhibits from, e.g., 50% to 100% identity across the full length of the candidate sequence or a selected portion of contiguous amino acid (or nucleic acid) residues of the candidate sequence. The length of the candidate sequence aligned for comparison purpose is at least 30%, e.g., at least 40%, e.g., at least 50%, 60%, 70%, 80%, 90%, 95%, 98%, 99%, or 100% of the length of the reference sequence. When a position in the candidate sequence is occupied by the same amino acid (or nucleic acid) residue as the corresponding position in the reference sequence (e.g., GJB2 amino acid sequences, coding sequences, nucleotide sequences for GJB2 gene regulatory elements (GREs), or any other sequences described herein), then the molecules are identical at that position.

An expression cassette of an isolated nucleic acid sequence described herein (e.g., the expression cassette of the isolated nucleic acid comprising a nucleotide sequence encoding a GJB2 protein) may further comprise a promoter operably linked to the coding sequence (e.g., GJB2 protein coding sequence). A “promoter” refers to a DNA sequence recognized by the synthetic machinery of the cell, or introduced synthetic machinery, required to initiate the transcription of a gene. The phrases “operatively linked,” “under control,” or “under transcriptional control” means that the promoter is in the correct location and orientation in relation to the nucleic acid to control RNA polymerase initiation and expression of the gene. A promoter may be a constitutive promoter, inducible promoter, or a tissue-specific promoter.

In some embodiments, the promoter is a tissue/cell-specific promoter. A tissue/cell specific promoter, as used herein, refers to a promoter that has activity in only certain cell types. In some embodiments, the promoter used in the isolated nucleic acid described herein has activity in cochlear cells that normally express the GJB2 gene. Use of a tissue/cell-specific promoter in the isolated nucleic acid described herein can restrict unwanted transgene (e.g., GJB2 gene) expression as well as facilitate persistent transgene expression. In some embodiments, the expression cassette of the isolated nucleic acid comprises a tissue/cell specific promoter. In some embodiments, the expression cassette of the isolated nucleic acid comprises a GJB2 promoter (e.g., a GJB2 promoter for any species where cell specific GJB2 expression is desired). In some embodiments, the expression cassette of the isolated nucleic acid comprises a human GJB2 promoter. In some embodiments, the expression cassette of the isolated nucleic acid comprises at least 300 bp (e.g., 300 bp, 400 bp, 500 bp, 600 bp, 700 bp, or more) of any consecutive nucleotides of a human GJB2 promoter. In some embodiments, the expression cassette of the isolated nucleic acid comprises a promoter having 500 bp consecutive nucleotides of a human GJB2 promoter. In some embodiments, the expression cassette of the isolated nucleic acid comprises a promoter having a nucleotide sequence at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 5. An exemplary nucleotide sequence of 500 bp of a human GJB2 promoter is set forth in SEQ ID NO: 5:

ACCTGTCTCCCGCCGTGGCGCCTTTTAACCGCACCCCACA
CCCCGCCTCTTCCCTCGGAGACTGGGAAAGTTACGGAGGG
GGCGGCGCCGCGGGCGGAGCGCGCCCGGCCTCTGGGTCCT
CAGAGCTTCCCGGGTCCGCGAACCCCCGACCGCCCCCGAA
AGCCCCGAACCCCCCAAGTCCCCTTCGAGGTCCCGATCTC
CTAGTTCCTTTGAGCCCCCATGAGTTCCCCAAGTGCCCCC
AGCGCCCTGAGTCTCCCCCGGTTACCCCGAGCGCCGCCTC
CCCCAGCCCCTTGGCGGCCCGGGTGAAGCGGGGGCGGCTG
AGAGTCGGGACCCCCCAGGAAGCGGCGCCCCAGACCCCGG
CTCCGGCGCTGTGCCGTGGGCGGGGTTCAGGGATGGCTGT
GGTCGTTGTCCTCTGTACTCCGCATAGTGCGAGAGGACTT
GGCATTTATGAGCGCTTCTTTAATTTTTTATTGTTAGAGA
AACAGGCATTCCTCCAAGGA

In some embodiments, the expression cassette of the isolated nucleic acid comprises a GJB2 basal promoter (e.g., a human GJB2 basal promoter). A GJB2 basal promoter is a promoter region of a GJB2 gene highly conserved across different species (e.g., human and mouse). The GJB2 basal promoter has been previous described, for example, in Tu, Z. J., and Kiang, D. T. (1998). Mapping and characterization of the basal promoter of the human connexin26 gene. Biochim. Biophys. Acta 1443,169-181; Kiang, D. T., Jin, N., Tu, Z. J., and Lin, H. H. (1997). Upstream genomic sequence of the human connexin26 gene. Gene 199, 165-171; and Castillo et al., DFNB1 Non-syndromic Hearing Impairment: Diversity of Mutations and Associated Phenotypes, Front. Mol. Neurosci., 22 Dec. 2017, each of which is incorporated herein by reference. In some embodiments, the expression cassette of the isolated nucleic acid comprises a GJB2 basal promoter having a nucleotide sequence at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 47. An exemplary nucleotide sequence of a human GJB2 basal promoter is set forth in SEQ ID NO: 47:

GGGTTCGGGGCTTTCGGGGGCGGTCGGGGGTTCGCGGACC
CGGGAAGCTCTGAGGACCCAGAGGCCGGGCGCGCTCCGCC
CGCGGCGCCGCCCCCTCCGTAACTTTCCCAGTCTCCGAGG
GAAGAGGCGGGGTGT

Examples of constitutive promoters include, without limitation, the retroviral Rous sarcoma virus (RSV) long terminal repeat (LTR) promoter (optionally with the RSV enhancer), the cytomegalovirus (CMV) promoter (optionally with the CMV enhancer) (See, e.g., Boshart et al., Cell, 41:521-530 (1985)) the simian vacuolating virus 40 (SV40) promoter, the dihydrofolate reductase promoter, the β-actin promoter, the phosphoglycerol kinase (PGK) promoter, and the elongation factor 1-alpha 1 (EF1α) promoter. In some embodiments, the promoter is a chicken beta-actin (CBA) promoter. In some embodiments, the promoter is an enhanced chicken β-actin promoter. In some embodiments, the promoter is a U6 promoter. Since the CBA promoter is constitutively active in all cell types, using a CBA promoter in the isolated nucleic acid described herein leads to promiscuous expression of GJB2 protein in all cell types, including cells that do notnormally express GJB2 protein (e.g., hair cells of the cochlea). Accordingly, in some embodiments, a CBA promoter is not used in the isolated nucleic acid described herein.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Many other promoters have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)).

In some embodiments, the isolated nucleic acid comprises a gene regulatory element (GRE) (e.g., GJB2 GRE). Gene regulatory elements, as used herein, refer to a variety of DNA sequences that are involved in the regulation of gene expression. For example, a GRE may rely on the interactions involving DNA, cellular proteins (e.g., histones), and transcription factors to regulate gene expression.

In some embodiments, the isolated nucleic acid comprises gene regulatory elements which are cis-regulatory elements (e.g., cis-regulatory elements for the GJB2 gene). Cis-regulatory elements are regions of non-coding DNA which regulate the transcription of neighboring genes. Cis-regulatory elements are found in the vicinity of the genes that they regulate. Cis-regulatory elements typically regulate gene transcription by binding to transcription factors. In some embodiments, the gene regulatory elements impart cell-specific gene expression capabilities (e.g., cell specific GJB2 gene expression). In some embodiments, the gene regulatory elements are cis-regulatory elements associated with the GJB2 gene.

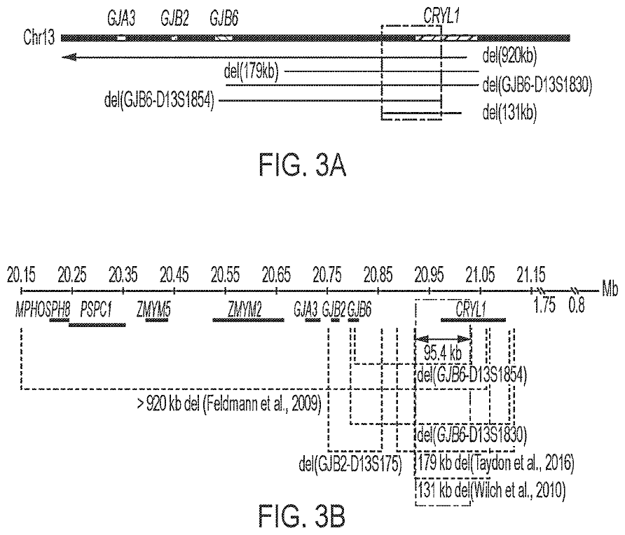

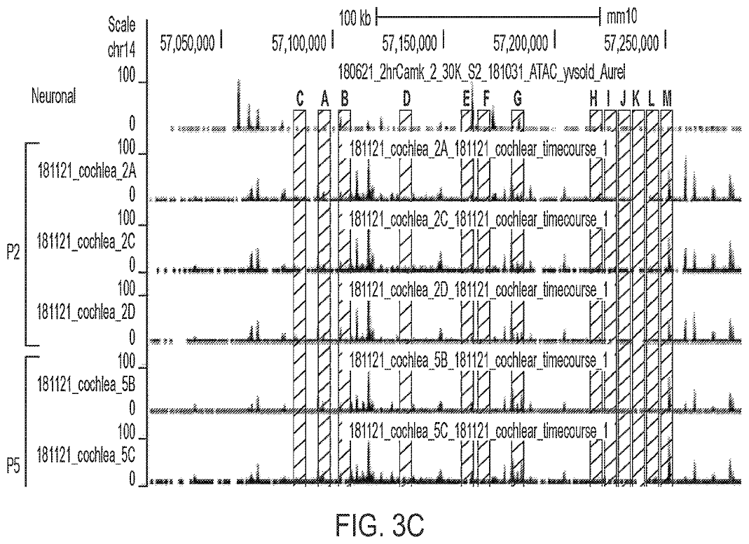

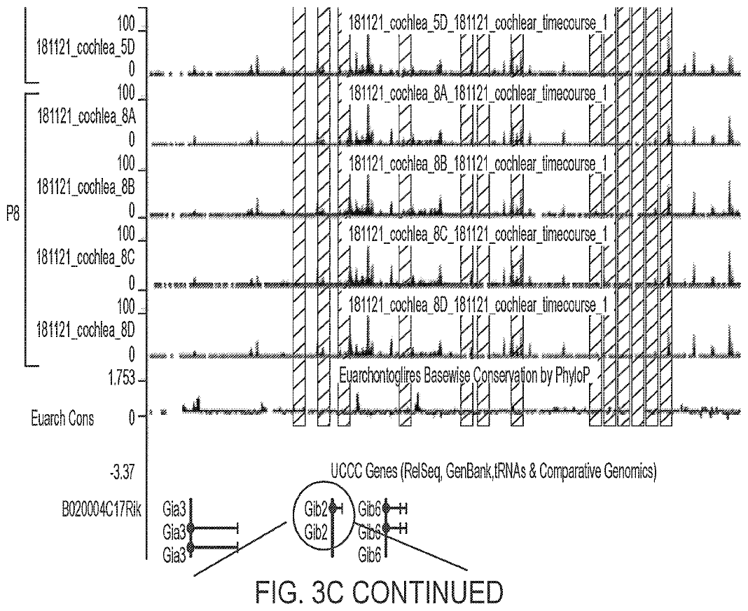

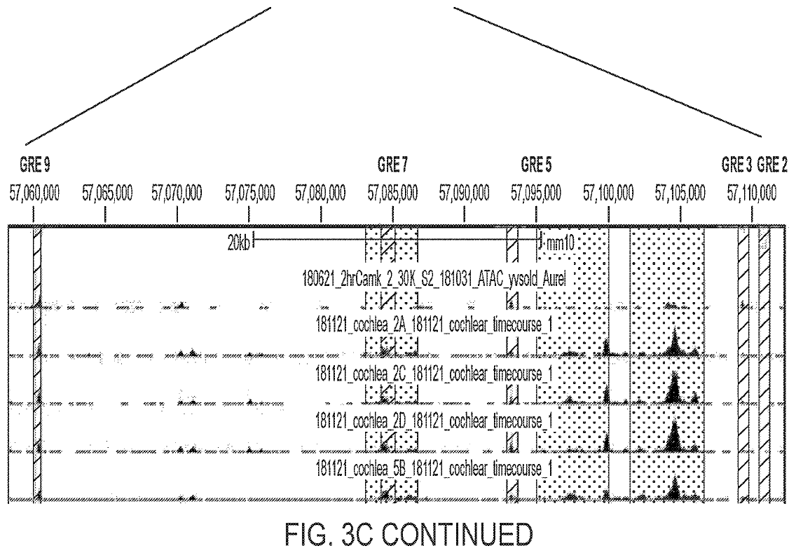

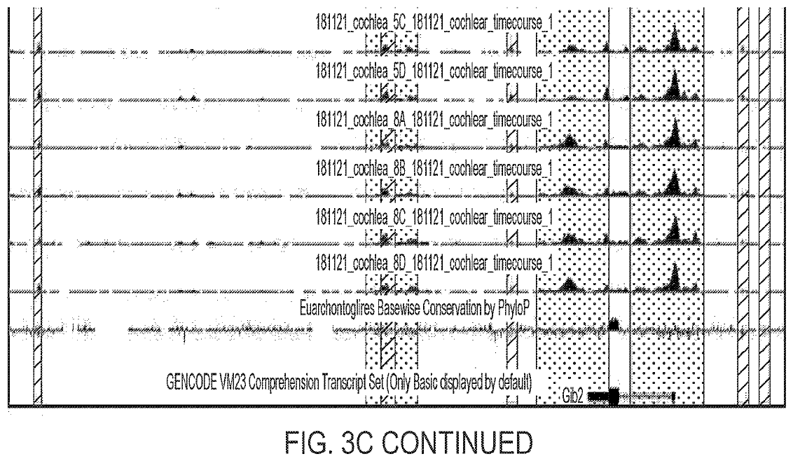

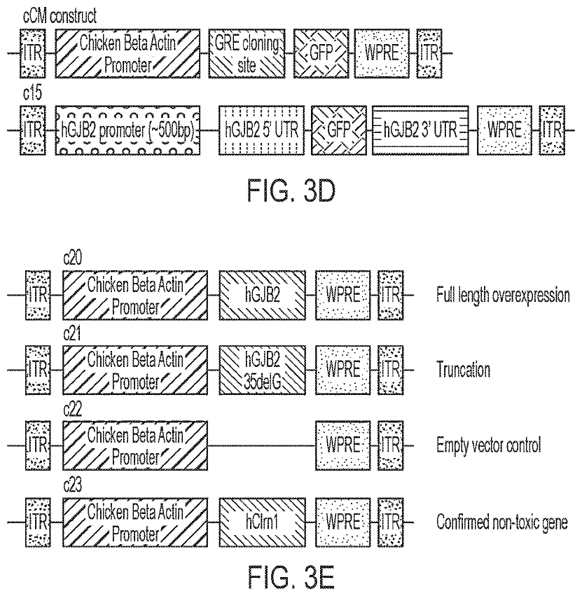

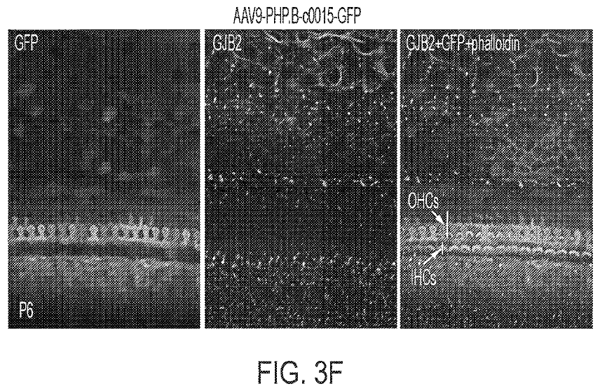

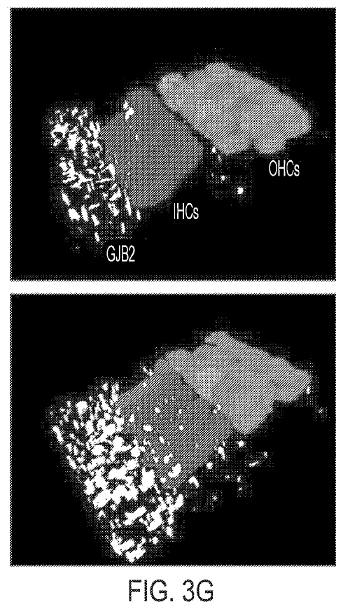

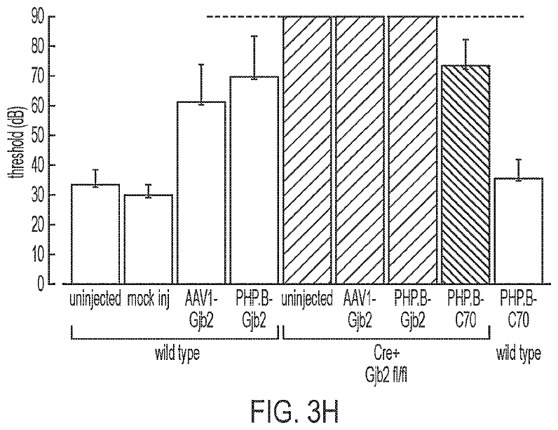

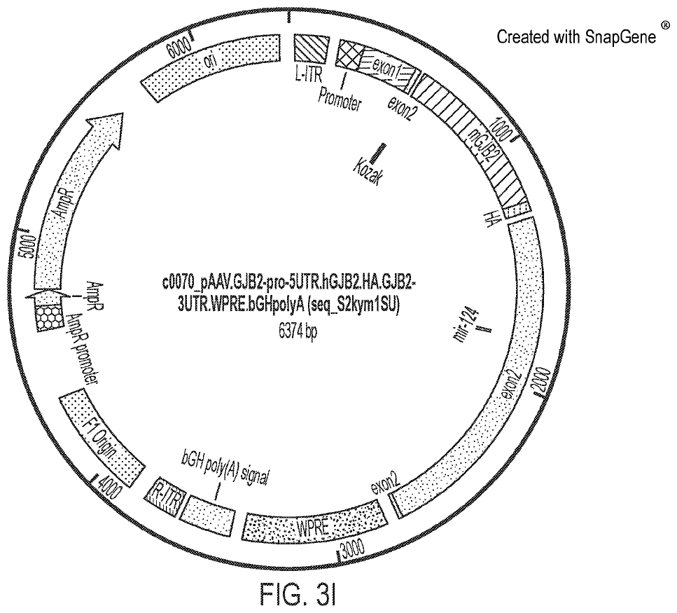

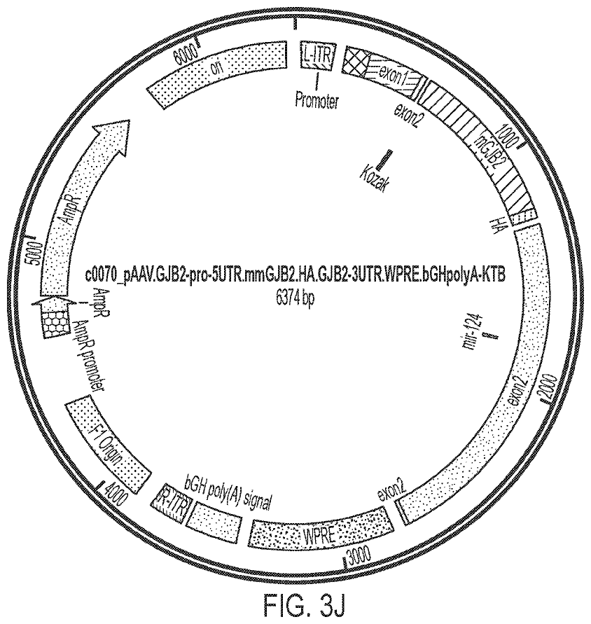

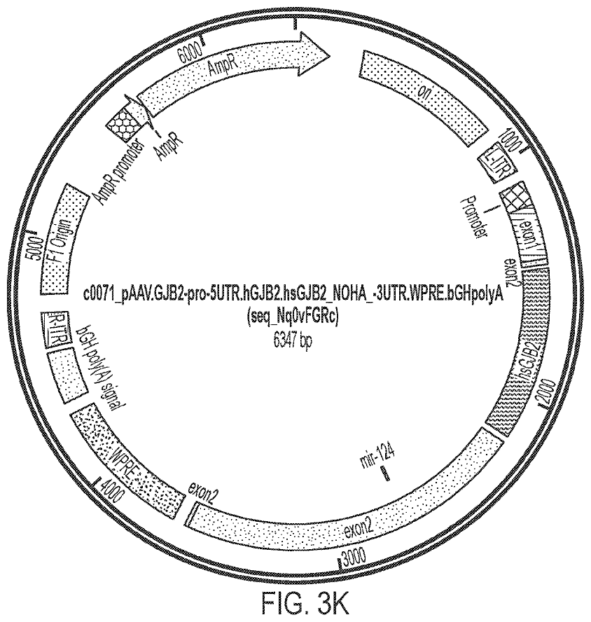

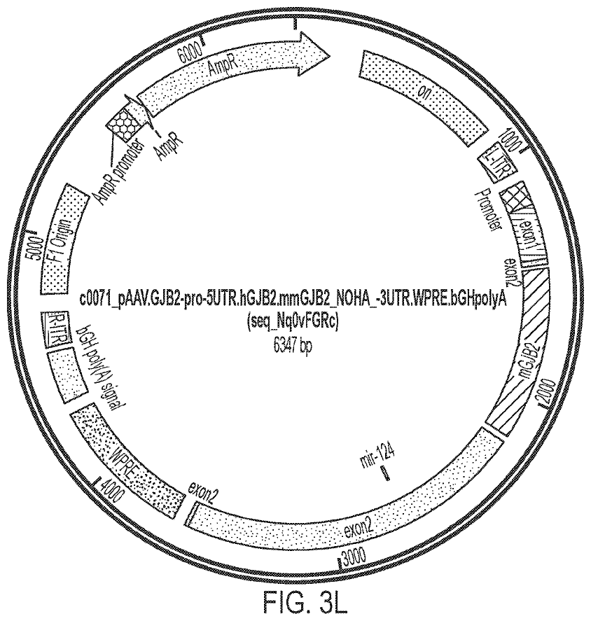

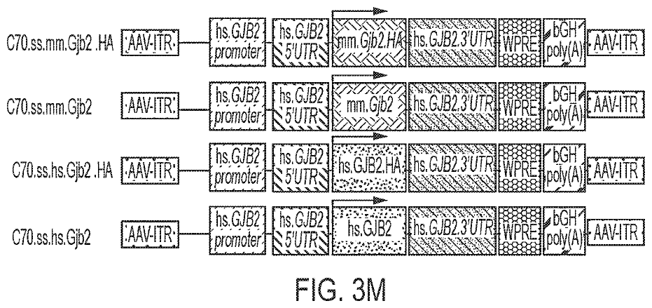

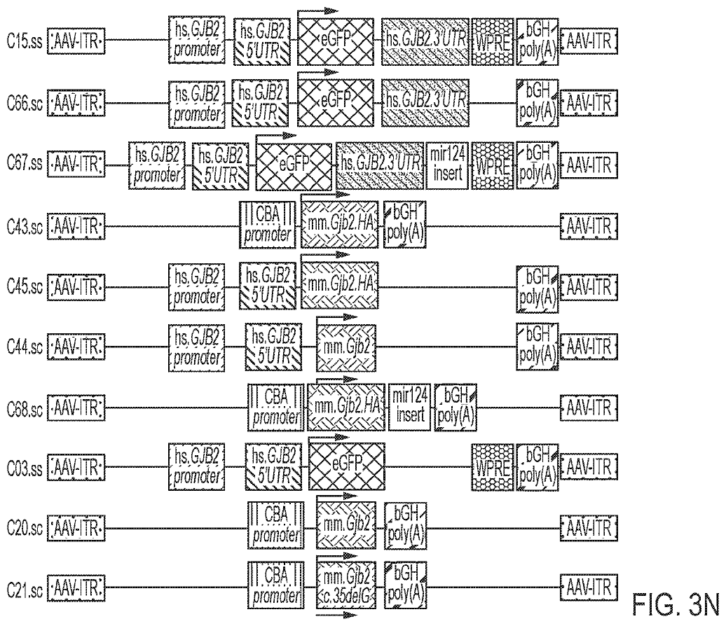

In some embodiments, the cis-regulatory elements of the GJB2 gene are enhancers. An enhancer, as used herein, refers to DNA sequences, which are located more distal to the transcription start site as compared to a promoter, capable of interacting with site-specific transcription factors to regulate gene expression in a cell-type specific manner. Enhancers confer cell-specific gene expression regulation by binding to the collection of transcription factors in a cell, which leads to transcriptional activation or inhibition through various mechanisms, e.g., recruitment of epigenetic enzymes that catalyze post-translational histone modifications, and recruitment of cofactors that promote DNA looping. Enhancers can be identified in the vicinity of the gene they regulate, or at a distance of hundreds of kilobases from their target genes. Multiple enhancers can act additively and redundantly to regulate gene expression (e.g., Doane et al, Regulatory elements in molecular networks, Wiley Interdiscip Rev Syst Biol Med. 2017 May; 9(3)). In some embodiments, the enhancers described herein are enhancers capable of regulating genomic GJB2 gene expression. In some embodiments, the GJB2 enhancers are identified in the transcriptionally active sequences of the GJB2 gene. A transcriptionally active sequence, as used herein, refers to a region of DNA in a chromosome in which the DNA is in open chromatin confirmation such that the sequence is exposed, thereby allowing binding of transcription factors and transcription to take place. In some embodiments, the GJB2 enhancers are identified within approximately 1000 kb of a genomic GJB2 gene (e.g., within 1000 kb, within 900 kb, within 800 kb, within 700 kb, within 600 kb, within 500 kb, within 450 kb, within 400 kb, within 350 kb, within 300 kb, within 250 kb, within 200 kb, within 150 kb, within 100 kb, within 95 kb, within 90 kb, within 85 kb, within 85 kb, within 80 kb, within 75 kb, within 70 kb, within 65 kb, within 60 kb, within 55 kb, within 50 kb, within 45 kb, within 40 kb, within 35 kb, within 30 kb, within 25 kb, within 20 kb, within 15 kb, within 10 kb, or less upstream or downstream of the GJB2 gene). In some embodiments, the GJB2 enhancers are identified within approximately 200 kb of the GJB2 gene. In some embodiments, the GJB2 enhancers are identified within approximately 95 kb of the GJB2 gene (e.g., regions C-M listed in FIG. 3C) In some embodiments, the GJB2 enhancers are within the regions of DNA sequences near the GJB2 gene (FIG. 3C) listed in Table 1.

TABLE 1
Human and mouse DNA regions that include GJB2 enhancers.

| Region | Human | Mouse |
|---|---|---|
| A | CTTTGTGGATGGCTTGGTGGCCTCACTGTCA | CCAAAAAGGGACAAAAACAGACAAACAAACAAC |
| | GGCTGGCACTGATGGCTCAGTTAGCATATCT | ACCAACACAAACAACAACAGCACTAAAACGAGT |
| | GTTTTGATAAGTGCTGCAACAGTGCATTATA | CTCTGCACCTAGGTCTTCGCACGCAGGCTGGTA |
| | ATTGTGGGCTGTGGTTTTAATTTCAAAGTGT | GTCCCACCCTCAGGTAGGGCCTGTTTGGTTAAC |
| | TTCTTAAAAGACACATTATTTTAAAATGACA | GATCCGTGTCTGTTTTGATATGTGTTGCAAGTG |
| | GAAAATTCAACTCCCTCGGTTACTGGCCCAG | AGTGTTGCACTGTGGACTATGGTTTTAACCTTG |
| | CTAAGCGACGTCACTGCATTGCAGTTCAGCG | AAGTGATTCTAAAATAAATATATGATGAAAAAT |
| | CTGAAGCTTGGGAGAGTCCCACACTCCTTAC | GACGGAAAATTAGCTCAGCGGTTCACCAGTTGC |
| | TGCAAGCGGATGTGGAGAGGCCAGTGGATAA | TGGTCCAAGGAGCCACCTGATGGGGGTTTTGCC |
| | TCTCCTGTGAGCCCATGGCCTTCTTTTCATC | TTGGGTGGCATCACAGTGTATCCTGTCTGAGTG |
| | CCAGGATGTGAATTGTCTTCACTGATTCATA | ACACAGTGTCTATATATGGCCTGTGCCCTAGAT |
| | GTTACACCCTGCCTGCCACAACCAACGCTCT | GAGCCTCCATAAGCCAATGACCTTCTATTTCAT |
| | CCTAAACAAGATTCCACCCTCTCCACAATCC | CCCAGGGCAGGAACCTTCCATGGCTACACCTGG |
| | GGATGAATCATCTCTTTTCCACCCTTCAGAG | TCTGTCACAATCAACCCCTCTTTTGATTAATCC |
| | CTGGTAGTGAATCCTCCTTCTTCTTTTTCTT | CATCTTCCCGGCTGTCCTGACTCACTTGCTTCC |
| | AAAAGCATCCTCCTCTCCTCATTTTAGGCAA | ACCCCTTCCTTCCAAGCTGTAAAGAATCCTCTG |
| | GTTGCATCCCGTTTTCTGATGGACTCCAGAA | ACTCTTTCTTAAAAGCACCCTACCCTCCTGCTT |
| | GCAGGCTCGTAGTGAATGTCTTTCATGACCC | AGCAAGTTACATCCTGTTTCGCAGTGGACTCAC |
| | ACAGTCGCTGCCACGGGGCACCAAGGTCAGG | AGCAGGCGCAGAGAGAAGTCCCTCCTTGTCCCT |
| | CAGAAACCATCCAGTGCCACCTTGGTCAGAG | AGTGGCGGTGGCAGAGCACCAGGGAACCCACTT |
| | GCTAACAGGAGAGAGGTGGCCACGAAAGTTA | GCTGGAACCCACTCAGCTCTGCCTTGGACAGAG |
| | CATCAGATTGACATAGGCCTGTGAAACATTT | GAGATAGGGCCAGGGGCATGGGAATTAAGGAAT |
| | AGCTTCACTGAGCTTGGGAAAGACAACATCA | ACTGACATACACCGGTAAAACATCAAGTCCTAT |
| | TTGGAAAAAACAATATTTTAGCCCAGGTTCA | CCAACTTGGAAAGCAGAAACAGACAGGCTCGGC |
| | GCACTGACCCATTGATAATCCAGACTGGGAG | AGGTTCAGCCCTGACCCATTTATACCTAGACTG |
| | GCCCTTAGGTGAGCTGGTTGTCCTGCTACAG | TCAGAGGCCCTTTGGGAAGCTGGTTGTCCTCTG |
| | CACCCACAGCTCAGGCCAGTCCCGTCCCAAC | AACAGTCTCTCAGCTCCATGTGGTCTGCCCCCA |
| | AGCAGAACCACCGAGGACAGCAACATTCCGA | ACAGCAGAAGGATTGAAAAGCAACAGTGTTCCA |
| | TTTTAACAAAAGCATCTTATGGAATTAGACA | AGTTTAACAAAACAATCTGATTGGAATTAGACC |
| | TTCTTCATTGGCCCTCACTGAGTGGAAAACA | TTCTGTTCTTCCTTCCCCTTCTCCCGAGTGGAG |
| | GGATACTCCCCGAAGTAAACTCTCTCCTGGT | ATCAGGACATTGAAATAAACATCTACACACCTG |
| | TTACAACAATACACCTGGCCAAGAATATGGG | ACCCAAAATACAGAGCTGGAGGATCCCTTTGCC |
| | GCTGCAGGAGGAGGGGTTTATCCTTTGCCCT | TGCCTATAGCATCCACAGACTAGCCCAATTATT |
| | CTTCCACCTGCCAAACCCAGGTCATACACCC | ATCAACACAGAAAAAAAAAAAAACCCTCAATTT |
| | TTCTACAGACCTGTCCAGTTACCATCAGCTG | CTGCGTAAACTGTGCACTTGTTTATAAAAGTAC |
| | AGAAAAATACAGTTCCGAGAAACCCTATATT | TTAAGTGTTTGTTGAATTTGAGTTTACCGTGTT |
| | GTTATTTTATAAAGCTTGAGTTGAAGCTACC | ACCCAGGATGGCTTCTAAATCCATGCAGTTGGA |
| | TGTTTTAAAGATCCTTTTTCAGGAAGAGGAG | GTTAGCACAACATGGGGGTGGGGGTAGGGGGTT |
| | TAAATTAAGATTTACTCCCCAATGGGCTAGG | AATACATCTATAATAGCAGAACTCTGGAGGCTG |
| | GGGTCATGGGTTAAGAGGGGCTCAGAAGCAG | AGGTAGGAGGAGTGTGCTAACTTGAGGAAAACT |
| | GACGAAGTTGTTTTCAATATTCAAGTCAGAG | TTTCTGCAGAGCAAGACCCTGGCTCAAGAAAAC |
| | GAGGAGCTGCCCTCCTGGCCTCCCGACCCTG | AAACACCAAAAGAGACAAGAAAAGAAAAGAACA |
| | GGCGGTTACATGCAGCTTCCTACCGGGCCCA | GAACCAAAACAAAAACAAACAAACAAACAAACA |
| | CGCCATCCTGCACCGCCTGGAGGGCTGCCAG | AAAAACCAAAAAATGGGAAGGCCGGATTGAACA |
| | AGGCCAGCGGAGGAGTTGGTTCAGTTCCTTA | AACAAGGTCAAGAAGAGAGAGAGAGAGAGAGAG |
| | GGGAAGACACTAGGTGAATCACCAGGATCCA | AGAGAGAGAGAGAGAGAGAGAGAGAGAAAACTC |
| | GAAAAGGCAAAAGGGACTCTTCACCCCTTAA | CAAAAGAAAACCAAATAGCTGGGACATAGCTGT |
| | ATTTCTCCACCCTTAGGTGATGGGTGGTCGA | GGGTCCCGGCATATCTGATTGCAGCTGCTTGTC |
| | CCTTGCCTGGCTGTCCCCAGAGGGTTCCTCC | TTAAATGGCCTTTCTAAGTGGAAGGAGAGGTTA |
| | ACCCTTCTCACCAGTGTCTGAAATTGTGACC | AAATTTGACCTCACAAAGGGGTTAGGAGTACTA |
| | GACTGTGCACAGCAGTTTCGAAAGGGACTCT | AGCCAGCAGGTGAAATCGTCAATATTCAACTGT |
| | AAGGTCACATGGGGACACGGCCGTACCACGC | GGTGTAGGAGGTGATTTCCAGGCTGGCCTTAGG |
| | TTCTCAAGGCAGTCCCAGGTGCATGGCCACG | ACTAGGTCACACGCAGGTCCCTACCTGGCATGG |
| | GAACCCAGCTCTCAGCAGCTGTTAGTTAGGT | GACACCTGGAGATTGCCTTGAACCGGTGAATCA |
| | GAGCGCTGTTCGGGCTGCCTTCCTCCTCCAG | TTCGCTCCTGAGTAGAAGGGAGCTTCTCCATGT |
| | TGGGGCAGGATCGAGGCACTGATGGAACCGT | TTATAGTATATACTGCATATGACCCTTATTTGC |
| | CCTGAGGACGCGGGTCTCAGCCGCACACCAC | CTTAAAGGATACTTCGGGGAGCTGGTGGACTGC |
| | CTCTTCGCGAACAAGGGTCCTAAAAATTTTC | CTCTAGATGCTGACCCCACCGCACCCTCCACCC |
| | CTTCTAGGCGGGGAGCACAGCCCGGAAACAG | TTCTCATAATTCACTGGCTTTGCCCATAGTTCC |
| | ACCCTCGTGAAGTGTTTAGGAAAAAGGGAAG | CAAAGGACTCCGGGGTAAGTGTAGCCATGACTG |
| | CCACTGAAATCTTGGCCCCGGGGTAGGCCGG | AGCCAGGCTTCTCAGGACAATCCCGTGGACCTG |
| | GATCGGCTGGCTCCGCGTTAGTTCTAGGCAA | AGCAATGGGTCCCATTTAGGCCTACGCTCCCTT |
| | ACTCCGCCCAAATCTCTGCCCGGGGATTTTT | CCCTTCCATTGAGGCAGCACCAAGGGGCTGATG |
| | CTGCAGAAGCCGCTCCAAGAGGTAAAGGTCA | CAATTGTCCTAAGGGACAAGTTTCTCAGCAGCA |
| | GTTCCTGCAGCGAAGGCTTCCTGCTTCACCG | CGCCATCTGTGAACCTGTGCCTTCCCTTCCAGC |
| | GCGAAACGGAGCTTTGCTTCGAAGCTAAGCT | TGTAACGTCCCGCCTGGACGCAAATCCTTAAAA |
| | TTCGGTGAATTTAAAACGTTTGGTGGCAGTG | AGCATTTAAGGAAAGAAAAAAAAAAAAAGCAAT |
| | GGTCAAGTAGCCAGGCGGCTGCGCTAGAGTA | CAAAATCTCCACCCGAGTGCAGGTTGGGGTTCC |
| | CCCCGAAGGGACATCGGCGACACCACAAACC | CCAGCTCGCGGGAGCGGCTACGGCCGCGCGTTT |
| | TCGCGCTGGCGGCTCGCCCGCGCCTTTTTCC | TGGGCGGTCGCCCACGTCACCCCAGTGCTTTAG |
| | CCTCCCGCGCGCGCCCGGCCCCACTCGCACC | GTGGTAAAGGTCAGTGTCTTCCCACGGAGGCTT |
| | CCGGGCGGTGCCATCGCGTCCACTTCCCCGG | CCTGCTTAACAAATGAAACTGAGTTTTCCTGCT |
| | CCGCCCCATTCCAGCTCCGGAGCTCGGCCGC | CAGCTTTCGGTTAGCTAAAAACTTTTCAATGGC |
| | AGAAACGCCCGCTCCAGAAGGCGGCCCCCGC | GGCAGACAACGCAGCCAGGAGGCCTCGGGAAAA |
| | CCCCCGGCCCAAGGACGTGTGTTGGTCCAGC | TTCTAGCGAAGGAATACTGGCGACACGTCGCAG |
| | CCCCCGGTTCCCCGAGACCCACGCGGCCGGG | TCGTGCGCGGAACAGCCTGGCCCCCGCGTCCCT |
| | CAACCGCTCTGGGTCTCGCGGTCCCTCCCCG | CCCCACCCCGCGCTGTGCGGGACCTCCCGGCTC |
| | CGCCAGGTTCCTGGCCGGGCAGTCCGGGGCC | AGGCTGTGCGCGGCGGTGAGAGCAGCCGGCTCC |
| | GGCGGGCTCACCTGCGTCGGGAGGAAGCGCG | AACCCCGAGCCGGGCCAGACGCCTGCAGCCGAA |
| | TGACAGAAGACAGATTTGCACAGCGCAAGCG | AGAGGTTAGGCAAGCCCATCCCTCTTGGAGTCC |
| | GATGAGGGACTAAGATGTGCAGAGCAGGCTG | AGGATGCTGGGAAGACCTGGGCAGCCTGCATCT |
| | GGTGGGGACTCCCGGGGAGGTCTCCCCCAAC | ACCTCTCTCCGCCAAGCTGTTCGTGGGTTTTGA |
| | CCCCGCCCCACCTCGGGCACCCACTTCGCGA | GGGCTCGGTGTTCCACATTGCTTGGCTGTCTGG |
| | TTTTTGCAGAGGGGAGCCAGGTCAGAGGTGC | ATAGTTTTGAGAGGAGTTACGGTGGACATTCAC |
| | AGCCTGGTCCCCTCGCGCTCACGTTTTTACC | AAGAGCTAGCTACGCTTTGGGATACCTAGGCCA |
| | CAGGTCAGTTCGAAGTTAAGTGGAAATGATG | GCTAGCTTCACCTTACTACTTGCAACCCGAGTC |
| | ATTAATCCTGACAAGTCAGATCTGGCCTCAG | CTACAGCTGCCAGGTTTGGAATGAAAACGGCAC |
| | AATGGATTTCCCGTGATTGCCACCATTATTA | ATCCCCACAAAGTTCCTTCAGATTAGCTTTACA |
| | GCATTGACTTTTCCTTGAAAAATTGGCGCCC | CGCAGTGAAGAGACTGATTCATTCTGACAAGGC |
| | CGTGGCCATGGGCCGACCTAGGCAGTTTCTG | CCGTCTGGTCGAAGGATTGGCTTTCAATGAAAG |
| | CAGGGACGAGCGTGAGTTTTGTACCGCGGTT | GACCATGGCTGAAGGTACATGCTTTCCCTGTAA |
| | ACCACCTACTTTCCAGCTCCAGGTCTTAGTC | AGCTGGCACATTGCCGCGGGCAGACCTGACTGC |
| | TAAGAGGGAGTGTCTGCTCATGAAGAGGCAA | TCTTGCTTGGGCAGAGGAAGGTTGCACGCTCGC |
| | AGCCCCAGGAGCTGCGAAAAGCCTTGCATGG | TTGCTACTACCCCCACCTCCTTTCTAACTGTAA |
| | CCCATCTGAGAGATGTGCTGAGTCGGCTTGT | GTCTTAGTCTAAGAGGGAGTGTCTCTAAGGAAG |
| | TAAAAATGACAGGCAAAGCCTGTGGGGTGGG | AGAGCCTCGGATCTGTGTCCAGCCCTTCAGAGA |
| | GCAGCTTTCTTGGCCTGAGCGCATCTTGGTT | GAGAGAGATGTGCTGAATCAGCTTGTGTGGAAT |
| | GAGCCAGAGGTGACTTGGGGTGGGGAGTGGG | AACTGGCCAAGCAAGATGGGGTGGTACAACTCC |
| | GCGCCGGTTGGTGGGTTCTCCCTTTAATTTC | CTTGGCCTGAGCACATCTAAAGATGAATCAAAG |
| | TCAAAGGCTGTGGTGTTTATGAGTCTGTTGG | AGGAGATGAGGTAGTGGCAGCAGGCAGGGGTGG |
| | AATCCTGGTTGGGTTGGAATGAAGGAAGGTT | AAGGATGTTGGCACCTTTAGCTTCTCATGGGTC |
| | CTAGAACCATTGTGGGAAGCTCGCTAGTAAA | GTACAGTTTCCAGTCAATTGGAGCCCCTGTTCA |
| | GATGGTTTGGAGATCGGAAGTTGACTGACTT | GTGAGGATGACAGAAGCTTCTAGAATCATTGTA |
| | TCCCCCATTGAAAAATGTCACCTGAGATTTT | GGAAGCTGGCCAGTAAAAGATAGGTTGGAGATC |
| | AGTGCCTGTATCACGATTATAGGCTCAACTT | AGAACTGCTTCACTTTCTCCATTGAACAATTTC |
| | TCTTTTCCTTGTTTTCTTTGATTTAGTTCTC | TCCTGAGGGTTAGTGCCCACGTTATGATTACAG |
| | CTTATGTGCAAAATTACTGTGTGATGTTGGC | CTTCAGCGTCTAGCTCCCTAACTTGCTTCTACA |
| | TAGTCGTATTATCACAGCCACTCCGTGTTTT | GATTCGCCTAATGGCTGTGTGTTGGCTGATGGT |
| | CAGGATTTGTAGCTGGAAGTCCTATAGCACT | CACAGGTGCTGGGAATATTAGGATGTATCGCTA |
| | TAAGTCTTCACTTACAGATCAGCGCTTGCTT | GCTCATCTCCTCCTCTGTTCCAGCCATCCCTCC |
| | TTATTCTGTTTTGTGTGATTTCTGCTGTTTT | TTGTTTCTTGTTTTCTCACCAACTAGACCAGAG |
| | CCTGTGAGTTGGTGTTTTCTTCCCAAGTAGG | GCTCCTCTAGGGTAAGAAATGCTAAATTTATTT |
| | CTCAGGACTCCTCTAGGGCAGGACATTATAT | GTGTATGTGTATTCTCCAGAGGGGGAGAGGGGA |
| | GCATGTACATAGTGTCCTCCAGTGTAGGGGA | GAGGGAAGGAGAAGGGAGGGGAAGAGAGGCAAG |
| | GGAGAAGGAGGAGAGGTGAGGTGGGAAAAGG | GAGAAGGGAGAAGGGAGGAGAAGGGAGGACAGG |
| | GTGAGGG (SEQ ID NO: 6) | GGGACAGAGGAAGCTAGAAAAGAGCTAGGA |
| | | (SEQ ID NO: 7) |
| B | TAATCCAGATGTTAACACTGAAACTTCCAAG | CATGGAGAGAGATGGATAACTGAGATTTCTGGG |
| | CAGGGGAGTGAAATGAGACTTTCACTTTTGA | CAAGAGATGAAATGGGCTGAATCCCACTCCTGA |
| | CTTCGTATACTCCTGTATTATTTAAGTGAAA | CTGCACACACCTCTCAGTGATTTAATTAGAAAT |
| | ATGTATTTATATATTCTATAATTACAAAAAT | AAAAACAAGTCTCTACATTAACATTTACATAAG |
| | CACATTGGTTGCCTTTTCATTTTGAAATGAG | TAACATCAGCCGTCTTTTCCATTCAAAGTGACT |
| | CAAAAGTGACAGGGCTGTTAAAAAGCTAAGT | GAAGGAGATGGTGTTGTTAAAAGATTGAAATTA |
| | CACTTGAGCAATAACGTGATGTCCAGAACAG | GACAGCAGCAACACGTCTAGAAGAGCATCCCTG |
| | TGGTTCCATGGCTCAGCCATGTCGGGGGCTG | GGGCAGGGTTCTGCCTCAACACCACACAGCACT |
| | CACTGAGGACAGGGGGCCATCTGCCTTCTAG | ACACAGCACCACACTTAGCACAAGGCTCCTCGT |
| | GAGGACACTGTGGACTGGAATATTGTTCCTG | GGCTCCTCATGTCCCTTCAGCAAGTCACCAGTG |
| | CCTTGAGGAGGAGTCTCCCAGCACAGTTACT | CACCAGGAGGCGTTGGGGAGGGAACTCCTGACC |
| | GCTGCTTGACTGTCAGAGCATGCGTTTTCTT | ACAATCACAGCCTGAGGGTTGGAGTTGTGTTTC |
| | AGGGAAGTTGAAGGCAGCCTGTATCTAGTAA | AGTCATCCTGGGGGGCAGGGGGAGCTTAAACTC |
| | GGTGGTATGCAGTAGTTGCTTAATGCTGAAT | GTTGGCATTTACTAGGGCAGTACACAGCAGCCG |
| | GTGTGAAGGAATGTGGGGCTGTGGAGCAGGA | CTCCACGTTGAACGAGTGGATGATCAGCCTGAG |
| | GGATAAAGTCTGAACTTGGACCTGTTGTTCT | AATCAAGGCTGGGCTGAGCTTGGCTCTATCCTC |
| | CAGCTATTCGAAGCTTTCTCAAGTGGAAAAT | AATTATCTGCAGAGCGCCCTGGTAGAGAACAGA |
| | AGACTGACTTTGGGTCCATCAGAGGGCAGAA | TCTGCCTTTGAGTTTCCAAGTGAGAGCGGAGCA |
| | CAAATGCTGGAGAGCAGATGCTAGAATTCCG | AGGCTGGGCACAGAGCAGGGTGGCAAGGTGGCT |
| | TCTTAAAACCATGAATCCTTACAGCGGCCTG | GCTGTGGGCACAGCACAGAAGATACTCAGGGGC |
| | CGTGGCCTGCGCCATCTGTCCCAGCCACGCC | ATAGATCTTCCTGGTGGCTGCTTGGTCTCATGT |
| | CTCCTTGGCCCCATCTCCCCCTTTCTCGCCC | TGGTCAGGTCACCTCCATTTTTGGCCTCATCAT |
| | TGACTCTTTGGCATCCTGGCCTTTCCGTCTC | CTTCTGACATGCACCTGCTTCATGCGTCTGCTT |
| | ACTGGGATGCTTCCCTAAGAGACTCGTGTGG | CCTGGAACCCATTCCTGGCTTTTTGTCTTAATT |
| | TTTGCTGCCCTGTATCCTCCGGATCTCCTGA | CTCTGAGGCAGGTGGCTCCATTGCTTGTCTCCT |
| | CCACCCTATGTTAGTTACATTGCAATTTCCC | TTAGGTTTCATCTAAGAGGGACCGTCACACACA |
| | GTTTCCCTCATGACGTCTTATTTTCCTCCAT | GCCTGTGTGGGCATCATGCTGGTGCCTGACAGT |
| | TTAAATTACCTGCAGCAGGTACCACCTACAG | CCTCTCTCTCTCTCTCTCTCTCTCTCTCTCTCT |
| | GGATCTGTTGAGAGTCGGCCTCCTTCAATGT | CTCTCTCTCCCCCCCCCCTCTGCTGTGGCTTTG |
| | GAAGCCTGATGTTTTGTTCTGTTCACAGCTA | GCCTCTGCAGAAACAATCTATGGGATTTGTTGA |
| | TGCCCCCAGCCCCTAACAGTTGGTGGCAGTC | TATGCTGCCTCCTTCAACACAAAGGCTTAAGTT |
| | AGTAAATATTGCCTGGGAAAACGAATCATTA | GTATTTATCAGCTCCAGTCCCAGGGAATAATCA |
| | GCCATGTGCAGAAATGGAACAGCGTCTCACC | TGTCTGGTGCTTAGCTGGTGCTCAGTAGATAGC |
| | AAGTTGGGGTTGCCCCTGGACCCTGTGAACA | AGCTGATGAAAAAAAATCAGGAGGGATACGTAG |
| | CTGGGGCAGCTGGGGTGTTCCTACTGTGCTT | GAACTGACCACAAAATCTTGTGGGGGTGCAGTT |
| | GTTACCGGCTTCAGGAATCAAATGCACTAGA | ACACCACGGACTCCAGCAGTGTTGCAACAGATG |
| | GAATTGTAGAAGTGCGGTCCACATCCTCTGT | TAGGTTGTGGGCCTGTGGAGTTAGTCTTCATTG |
| | GTGGTAGGACCAGCTGCTGTTGGCCTCTGAG | TGGGAGGGGCAACTCCACAAGGCCTATCAACAT |
| | CAGGATCTCTTACCTCTCTGAGCAGTGCCTT | AACCTCCGAGGGGTTGGACTACTCTTGCTGGCC |
| | CCTGTTGCCCTCAGCAAGAATAACACTAACA | TTCGATCTTGACAATTACCAGTGCCTTCTTCAC |
| | GCCTAGGACTTCAGAGCACTGCTGCGAGGTG | AACCCCTCCCCCACCCCTGCACAGGTGATGACT |
| | CAAATGAGGTGATATGGGAAAAGCATTTGGT | TGATGGTTCTTAAGTTGCAATAAGAATGACAGG |
| | GAGATGTATGGAAAGTGTAGAGACCCTGACC | AAGCAAGCAGGAAGCAAGAGATGTGATATACAC |
| | AGATGAGTCAATGGCCTTCTTCGTTACTCTG | ATTAGGTCGTATGGAGACCCTGACAGAGCAAAC |
| | TTGACCTTTCTTTAATTACAGAGTCGCATAG | CTGTAACATTCATTCTTACTGTATTAGCCCCTT |
| | CTGTCACCACCTTATCCTTTTTTGCTGCTAT | TCTTAGTCACTTATTAATATTCATTTAGTCATT |
| | ATTTGCCCCCAGCCATTCCTCTCCCGGCTTA | TAGTTTTTGCTGTTTGCTTGATGCAGAGTCTCA |
| | TGTGGCTAGACTCACCTGCCTGTGCTGCAGT | TGAAGTTCAGGCTGGCTTTGAACTAAGTATGCA |
| | TACTCCAGGCTTTGTGTAAATGTGCATTTTT | GCTGAGGATAGCCTTGAACTTCAAATTCTCCTA |
| | TTCCAGCCCCCAGTTTATCAAGCTTTGCTTG | CCTTCATTTCTGAGCCATTGGGAATGCAGGCAT |
| | AGTCACTTGTATCTGAAATACCATCTGTCAC | CCACCTTGGAGCGCCATTTCTATTTATTTACTT |
| | TCTTCCAGGTTGGGATCTGTCTAGTGGAAAA | TCTCTAAGGCTGGGGATGGAGCCTATGGCTGTG |
| | CAGATGACAGTCATATGTTACTTAGTGCTTT | TGTGGTAGGCACAGGCTGGGGATGGAGCCTATG |
| | ACTATGTGGAGAACGTTTACATAAATTATCT | GCTGTGTGTGGTAGGTAGCATTTTGGCATTGAC |
| | TATTTCATTGCCACTAAGCCGGGGAAAGATT | TCACTTACTCTCCAGCCCTTGATTCTTTTGAGT |
| | CAGGAAACCCATTTTAAGATGAGGACACTGA | TACAGAGTGATACCATTGCCTGTCACTCATCTT |
| | GGTCAGGGTAAGTGAGTGAGCTTTTACCCAC | TACTGTGCTTTTGTGTATGCACCCAGCCCCCCT |
| | CTCTCAGCTGCTCTCTAGTTGTCAAAGACCA | TCCTCTGTTGACCTGGCTGGTCTCTGAGGTCAC |
| | ACCCGTGGGGGTGGCTCAGGCCCGACCCCTG | TGTGTTATGTTTATTTCAGTGTCAACCTGCACA |
| | CAGCATATTCCTTGGGGCCTCCCAAGTGGGC | CTCTCAAGCTTCCGGTTAATTGAGCTTTGCAGG |
| | CCGATCTGCTCACCCCAGCTGTGACTGTCTT | AGACATTCCTACTTACTCTGTCATTCACCATGT |
| | TTGACAGGAGGAGGGAGCAGCGAGGCTGCAC | CACTCAGGGTCTACTGAGTGGGAGAGAGATGAC |
| | CCACTGCTCATAAAAAGCAGAGCTTGTCCAC | ATATTAATGCTAATATCATTCTACTGCCCTAGG |
| | GCCGAGGGCTCGGCTGGGTGGGAGGCCGCTT | TGGAGGAGAGGGTCTGTGTGAATCACCCCATTG |
| | CCACAAGGCTTTTTCTTGCTCCATACAAAGT | CTTTTCCTAGGGGTGGGGAGTATTTAGGAAGCC |
| | GCAGACTGATGCTTTGAGATATAGTCAGGAT | CACTGTAAGGTGGAGAGCCTAGGCCAGGGTAAG |
| | TATCATTTTCAGAGCTCAAGCTCTAATTTCC | CACGGAGCTCCCTTCCACCCGTGGCCACCCATT |
| | AGGCATGTGACCAGACCTCTCTATCCATTCC | CAGCATTTGCAAGCTGCTCCCTGGTGCATCACC |
| | TACAAGTGGTCGAGAGTAGCCCATAATTATT | TAGTTAGAACAGTGGCACCTGAGACAGCTTAGG |
| | TTGGCTTGGTCTTTTAATAGCTTGAGAGTAA | CCTGGGGAAACCAATAGAACACTCTGTTGTTCC |
| | TAATCTACATAGCTTGTAGAAGTGAATGTAC | ACTTGGACTAGCAGTGGCCTGTCTCTCCACAGG |
| | TTATTTTAAAAGTTCTGTGTTTTTTGATGTT | GAGCACCACCCATGTTGGGGAGCATCACCTGTA |
| | GTTGTTGTTTGGGACAGGATCTTGCTGTCGC | ACCTCCAGAGTTCACTCACACCAAGGCTTCTTC |
| | CTAGGCTGGAGTGCAGTGGCACAATCTCAGC | TCTTCACAAACTGCCATCTGCTAGTATCAGGAT |
| | TCACTGCAGCATGGACCTCCCAGGTTCAAGC | GATCATATTCCAGAGGCCAAGCTTATGGCCAGC |
| | AATCTTCCCACCTCAGCCTCCTGAGTAGCTG | CCTCTCCGTCAGTCCTATGAAGTGGTTGTTGGC |
| | AGACTACAGGCACATGTTACCACGCCTGCCT | AGTTTGTAATTATTTTGGCCCTGTTCTTTAATA |
| | GGCTAACATTTTTATTTTTTATAGAAACAAT | CCTTAAGAGTAATAATCTTCATAATGTGTAGGA |
| | GTCTCCCTATATTGCCCAGGCTGGTTTTGAA | GTGGAACTAGCCATTTAAAAAGCTGTGCATTCT |
| | CTCCTGGGCTCAAGTGATCCTCTCGTCTCAG | TTTAACAGGGTACGTCCAGGACACCCTGGCAGG |
| | CCTCCCAAAGTGTTGGGATTATAGGTATAAG | TGGGAGAGACTATTCACTTTTTCTACTGTCCAA |
| | CCTCTGCACCCAGCTTAAAAAATCCTATTTT | GTGGACGTGGGCTAAGTTGTATCCCTTTCGAGC |
| | CACAGTCTATGTGCAGAGCATTTTGGAAGTC | TAGGTTGTATGGTCCTCCATAAAAACATAGTAT |
| | AGGTAGAAACCATTTCCCATTTTCTATTACC | CACTGATGTTTAAAATGCCTTGACAGCCTCAGT |
| | TGGGTGATAGTTGACTGGTTTTTGTTCTTTG | GTGAAGCTTATAATTTAAAGGATGATAGTGTAG |
| | GAAAATACTTCTAATTATTGATGTGTGAAAT | CTTACCTGGGACACGCTTGCCTGGCAAGGTCTG |
| | GCTTTGAAATCCTTGGATGGAAATCTTGTAC | TCCCGTGGGAATAGACATGGAGGAAACAAAGAA |
| | CATGAAAGAACAGAACTGTTGGTGGTGTCTC | CATGGGCCACATGCTTCTACACACACACACACA |
| | TGGGAGAGGCTCACGAGGGCCGGGCAAGCCT | CACACACACACACACACAGAGAGAGAGAGAGAG |
| | GTGGGGGTAGCAGGCAGTCACTCCCATGGGG | AGAGAGAGAGAGAGAGAGAGAGAGAGAGAGAGA |
| | ACAGGCTGACCTGGCAGGCTTATTTCCCATG | GAGAGAGAGTCTTGCAAAGTTCTGCAGAGGACG |
| | GAAGTGGGCACTGAGGAATAAAAAGCAGTTT | GTTCTCAAAGTGTAGTCTTCACAGTGGAAGATG |
| | CAGGCCAGGTGCGGTGGCCCATGCCTGTAAT | TTTTAATTTTTAAATATAAAGAGGTTTGTTGTT |
| | CCTTGCACTTTAGGAGACTGAGGCAGGGGGA | GTTGTTTTCTGTGATACTGGTGTTCCAATATGG |
| | TCCCTTCAGCCCAGGAGTTCGAGACCAGACT | GGGCCCACACACGGAGACAGGTGTTTTAGCGCT |
| | GGGCAATATAGTGGGACCTCGTTTCTACAAA | GATTACACACTGAGCCTAAGGACCATGTAAACT |
| | AAATGAAAAAATTAGTGGAGTGTGGTGGCAC | GTGAGTTCCTCTGCTTCTTCTAGAAACGGAACG |
| | ACTCCAGTGGTCCCAGCTACTTGGGACGCTG | GAACTGATCCCGTCACCAGGACTTAGCATCCTC |
| | AGGTGGGAGGATCGCTTGAGCCTGGGAGGCA | CTGCTGCACTCTGACTCTCAGACCTTGCAGCCC |
| | GAGGTTGCAGTGAGCCAAGGTCATGCTATGA | TTAGGTTGGGGCTCACGGAACCTCTTAGAGTGC |
| | GTAACATTTTGAAGGTCCACTTCTGGGATTC | GTGGATTTGGGCAGCAGTGGTCTGTCTGTTCCC |
| | ATCCAGGAGCTAAACGGGTCATGTCCAGCCA | TCTCTCTTTATCAAGTTTTCTAGCCACAGGGTA |
| | ACTCAGCATTCACCAAGGTACGTTTCCAGAC | TTTTTTGTAACTGGAGCAGAATCCCAGAACATG |
| | CAAACACCACATTGTCCATAGACTGATATGC | TTGTAACATGTGAGCATACTTCTGGGATGCTTT |
| | CTCAAAAACCTGGTAGAGGTGGGCACGGGGT | AAGATATAAACTATGAAATATATGTATATACAA |
| | TAGGTAGAAATCATCTTCCTCCCTTCCTTCC | ATTAGTATAGCTGGGCATGGTGGTGTGCACGTT |
| | CCACCAAACTTTCTGGTGACAGAAGCTTTTC | TAATCTCAGTCCTTGGGAGGCAGAGACAGGCAG |
| | TGTAACTGGGGCAGAATGGGGTCAGACACTC | ATTTATGAGAGTTCTAGGCCAGTCTGGTGACAG |
| | TGGCAACTTACCCATTGGTGTTATGAAATAT | AGTGAGGCCCTGTTTCAAAGACAAAAACAAATC |
| | AAAACATTAATGTATTTATATAAAAAGTGAT | AAAGCCAGAAAAACTTACCATTGGTCACGTTAG |
| | AGATGAAATTAAAATTTGCTGTTCTATTAAA | AGTTTGGTATTCTATTAAAAACCTTATTTAATT |
| | ACCATATTAGATTTTAAATTATTATAGAGAT | TTAAAGTATACAAAATAATCATATTTTAATAAA |
| | TATATTTTAATGTTTTAAATGTATTTGATAC | GGGCATTTAGGGGTTTACAAAATTATATCAGTG |
| | ATTACAAAATTATTTTAGTTACAAGCATATC | ACAAGCATGAAACCACAACTCTTATTTATTGTT |
| | ATTAAAGCTATTCTTTATTATTACAAAATGC | ACAAAATGGCTTTCCAATGACATTCTTGGCAGG |
| | TTTTACAATGCTATTCTTGACAACAGGAAAA | AAGAAGTGTCCCCTGTTGGATTTGTTGACTGTC |
| | TACTTACCCTCACTGAAATATGTGGAGTACC | ATCTTGTAGGATACACATAAGGCATAGTGGTAA |
| | ATTTTTTGGAAACCATGTCAAGCATAATGGC | TGGTTCAACTTGCCCTAGAAAGGTTACATACTG |
| | AATATTCAGGTTCAATCTTCCTATAGATCTG | ACCTAAACTAGTTTCTTCTATTTCTTCCAAATA |
| | CTCAATATTTATCTAAACCTTAGCTTCTATT | TCCACATTTCTGTTTCCAGTTAAGAAGGCAATG |
| | CTTTTCACATGTTATTAGCTATATTTTCACT | CTGAAGAGGGAGGCAAACACACTTTCAAAAGTA |
| | TAAAAAATTGGAGGCTGAAGGGGTAAGCAAA | GAAAAACTTAGTTTTAATCAACAGGATTGGGAG |
| | CAAACTTTTGAAGTAGACAAAGCTCATCTTT | TCTAGAAGTTTCATTGGTTCTCTGAAAACCACC |
| | AATCAACAGACTTTAGAGTCCAGTCTTTCCA | CCATTTGGTTTCTGCACCATTGAATTGTCCCAT |
| | AAATCCATTTTAAAAGTGTATGGTCCTCTAT | GTACCACCCAGGAGAGAGACGTATAGCCTGTCC |
| | AATCTGTTTTTAACGACAGAAACTTCTCCCT | GGCAGTGAAATTCCCAAGCAAACCCATGAAGTC |
| | CCCCTGCCCCATTTTGTCCTCCCCATTAAAT | CCTATCTTCTGATGCTGACTGCAACATCCCACA |
| | GGTACTGTGTCAATAAAATTCCCAAGCGACC | GCTACAGAGTAGACAAACTGGTGGGGGGTGGGG |
| | TCTTTAAATCAGCGTTCTTTCCGATGCTGGC | GTGGGGTGGGGCTGAGTTAGGCTCATGGCAGGT |
| | TACCACAGTCATGGAAAAGGGAGATGTGTTG | GGCAGTTGTCGGCATATCCTATCTGTCTCTTAC |
| | GACAGGCCTGTCATTACAGGTAGTAGTTGGT | ACAAAATTACAGTTGACTATTTTAATTGAGGCC |
| | GGTACATCCAGTCTGTATTTCTTACACAAAA | TCTTCTTGTCAGAAGCCAGCACGAGACGCTTCC |
| | TTACATCTAAATATTTGACATGAGGCCATTT | AGTTTGTCTCACTTATGACAGGCAGTAGGGTTA |
| | GCTATCATAAGCCATCACTAGGAACTTCTAG | TAGCCCTGAGCCCAGCACGCCAGTGATGAATAC |
| | TCTGTCTCACTCGATTGAGGCTACAATGTTG | AATAGGTGGGCCCTCAGCCACACTGCAGGTTTC |
| | TTAGGTGCTATGACCACAATGAATACAACAG | CCATAACCCAAAGGCCAACATCTTAAAGACCCT |
| | ACAGCCTCTCAGCTGTGCTGCAAAGTATTCA | GTGAGATCTGGTTACACACCATGCTCACTTCAC |
| | TAACCAAAAGACCATATTTCAAATTAAATCA | ACACTGAACCTCTGGACTAGGAGGAATGTATAA |
| | TAGTAGCGAATGACATACCATTTACATATTA | TACTTTCCAGATCATTTTAGGAAAAAAAAGAGC |
| | CAATCTGAGCCTCTGAAACAGGGGGAACATA | CTATCTTATTTTAAGGTTTTCATTAAAAAAAAA |
| | TAATGGTATCCAGAACATCTTTACATCAAAA | AAGTACACAGCACTTGAAGTATTAATAGCTTTT |
| | TAACCTATCATACTACAAAGTTTTCACTTCC | TGTCCATTGTTGCACACGTAAACTATCAAAGCA |
| | AAAAAGTGTAACAGAGTTTAAGGCACTGGTA | AATAACAGTATGGCATTTCTTTACCTTTAGCTA |
| | ACTTTGTCCACTGTTAGAGATTAAAACTTCC | GGGGTAACTTGGGGGGGGGGACTTTCTCAGTGG |
| | AAAGCAAATGAAAGAACCAATGTTCACCTTT | CACCTTCCTCAGGACCGGGTTCCTCTCTCCTGT |
| | AACGTGGGGAAAGTTGGCAAAAAGAACCCCA | CCTCAGAGGAAGAGAAACAATGTGAGATCCCTT |
| | GGAGGACACCCAAACCTTCTCTGTGTCCTCT | TGTTTAAACTGTGAATGTATCCTCCAAGCTTGG |
| | GTGGAACCTGGCTTTTTTCTCTTGTCCTCAG | TCGCTACCAGCACGGGGTCTCAGTGGAACTAAC |
| | AGAAAGAAACAAATGCCGATATCCTCTGTTT | TTTAGAACCCATTAATACAGGCATAGAATTGGG |
| | AAAATATGAAAGTACCTTACACCAATAACCC | CCTTTGTTTGGGAGCTTTGGGGGAAGGGAGGCC |
| | CTAACAGCCTGGGGTCTCAGTGGAACTAACT | CACGGAGGCTTCTGGAGTTTCATAGGAGGCCTC |
| | TAAGTGAAAGAAAATTAAGACAGGCATAGAA | CAGGGACTTCAAATGGTGGCATTTTAGATGGGA |
| | TTAGGCCTTTGTTTTGAGGCTTTAGGGGAGC | ATGTTTGTCTTGGGAACTGCTGGTGGCTGAGCT |
| | AGAGCTCCATTGTGGCATCTGGAGTTTCACC | CTGCCGACTAAGCGACTAAGCATGGGTTGCCTC |
| | TGAGGCCTACAGGGGTTTCAAATGGTTGCAT | ATCCTCTCCCTCCATCTTTGCTCTAGCAGCCAG |
| | TTAAGGTCAGAATCTTTGTGTTGGGAAATGC | GCAATGCATTAGACTGGTCTTTTGGACTTTCCT |
| | TAGCGACTGAGCCTTGACAGCTGAGCACGGG | GAGCAATACCTAACGAAC (SEQ ID NO: 9) |
| | TTGCCTCATCCCTCTCATGCTGTCTATTTCT | |
| | TAATCTAACAACTGGGCAATGCGTTAAACTG | |
| | GCTTTTTTGACTTCCCAGAACAATATCTAAT | |
| | TAGC (SEQ ID NO: 8) | |
| C | AAGGGGACAGGACATCTCTTTCCAAAACTTA | GTAAGAGCCAATTAGGAAGTTCCAGGGTTAGTA |
| | GGTTTGGTGACTCCTGGATTTCACACTCTCT | AAGGCCAATCAGTAAGCACCAGGGTAAGAGCCA |
| | GACTGCTTGGGTGAGGGTGGAATGGAGGGCT | ATCAGTAAGCTCCAAGGTTAGTAAGAGCCAATC |
| | GTCCCCCACCCTCGCACCTGCACGGTGGCAT | AGTAAGCTCCAGGTTAGTAAGAACCAATCGGTA |
| | GCTTTCCTCCTACTCCAGGGAATTCCTCGTG | AGCACCAGGGTTAGTAAAGGCCAATCAGTAAAC |
| | GCCTCATGGCCTGGGCTGTTTCTGGCTTCAA | TCCAGGGTTAGCAAAGACCAATCAGGAAGTTCC |
| | GCTCCACGTGGCCTGGCCCCAGCGGTCTGGT | AGGGTTAGTAATGGCCAATCAGTAAGCTCCTGG |
| | CCACCTTGTACTCGGTGCCCCCGCTGCCCCC | GTTAGTAAGAGCTTCTGGTTTTGGTCCTTCAAT |
| | TGGCCTCAGCTGGAGTGACGCACCTCATCCA | CACTGGCCTGAGCACTCATGTGATTGGCTAGGC |
| | TGCGGGCCTGGCGTCTGGAAGGTGGCTGGGT | TGGCTAATCAACCAGCTGTGGGAATACTATCCA |
| | CTCTCGGGCTTGAGCACCATCATCTTAGCTC | GTGATGGGCTTGCAGACAGATGCCACAGCATGT |
| | CAACATGTCATTATTCCTTCCTCACTGAGGA | GGCACCTTTAATGTGGGTGCTGAGGATACAAAG |
| | CTTTTCTGCTTCCTAATTGGTTGTTGAAGAT | TCAGGTCTCTCCACGCTTGCATAGGAAACACTT |
| | GAGGCCCCCATGCTCTTTTAAGAAAACCTGT | TACCAAATGAGCCATTTTTCTCAGTTTCGATTT |
| | TGTGCCCCAGGCTTGGCTGTGATGGGCACTG | TATTTTATTTTTTGAGACAGGGTCCCACTGTAT |
| | ACTCATACAGAAGTAGAAAGGCCTGCTGAGT | AGCTCAGGTTGGACACAGACTTGTGATACTCCT |
| | CATCAACACTCGTGCGACGCCCTCGCATTTT | ATCTTGGCCTCCTTGACTACTGGAATTGCAAGT |
| | CATTAATGATGGCCTCCCTGCCACACGTGAA | GTGTGGCACCATGCCAGCTGGAAAGGTAACTTT |
| | TCACTCCAGCCCGAGATCTGAAACCAGGACA | CTAAGGTACCTCTTTCTAAAATAGATGTTGACC |
| | CACCCCAGGGGCGAGGTGACGCTGAGTGAGC | TTTTGTAAGGACAGACTAAACGCCCCCTGGGCT |
| | CCAGCTGTGTCCCTTTCATGAGAACTCAGAG | TGAGGCTGGCGCCATCCAGAACAGGGTAGAGCG |
| | CACAGGGCTCTGTGTGCATGGCCGTCCCCTC | TATTGAGCCTGGCAGGTTGAATCCATCTCCCAA |
| | CAGAGAGGAGGAAGTAAATGCCGGGATTAGT | ATGAAGAGGGCAGGTGGGTTTTGGGGGTTGATG |
| | GGAAGATCATTTCCTTCTATTTGCCTTGGCT | ACGAGGGAGGGGCAGAAAGAGGGAGACAAGACA |
| | TACGTCTTTCAGAATTCAAACACGTGCACTG | GAGAGTGTTACTCAGTCCAGGTACTCTCTTGAA |
| | TTGACCCTGCAATGGTGGAGTTTTTGGATTT | CTAAGAGCACACAGGGAAGAAGGGCCTCATCTG |
| | TCCTTCAGTCCGATTGCTAAAATACTTCCCT | AGGCCAAGGTGTCATTGTATCCGGTATAAGGGG |
| | CTCATGTGAGCTGTTGTGAAAGTCATCAGCC | ACAGGATCACCTCCTTTCATGTTGGAGCTCGTG |
| | AGATACCATTCTAAAAACAAAGAATGTGCTT | GATCTTACATTCTCTAATGCTTGACTAGATGTG |
| | CTCGTATGTTGCATGCTGGTTACTGAAATAT | AGTGGAGCTAGAACACGTATCTTCTCCTGGTCA |
| | TAGGGAATTACATAAAGGTTTTCTGGGGCAC | CCGCCCAGGGTTCGTGCGCTTTTCTTACTCGGT |
| | ATATTCAAGCTGAATGATAAAATTGAAGGTC | ACATCATCCTCATCGCAGTGGGCTGGTCTCTGG |
| | ACACAAAGCTAAGGTCTTTCAAATCCTGACC | CTGCCTCATCCAGTTTGTCGTCTCAGTTCATAC |
| | CAATTAGCTCTCTGTTAGCTCTCTGACTTTG | GGACACCCCCTGGCTTGTCAGTGCTGGCCCAGT |
| | GACAAGCTGTCTGGTCCTCTGAAGCATACTT | ACCCTCGGGCCTGAGCACCTGTGATGCCCCTGC |
| | TGTTCGCCCTGGGTAGGGGCCCTCTGTTTTA | CTCCAGCTCTTCCTCCCCAGAGTCTGCAATGCT |
| | ACAGCGTTTGGCAGATGAAAACATTTGCAAA | ATCATTCCTTCCCGGCCCAGAGACTTACGCTTC |
| | GCCAAAGGACAATGAAATCTACGGAAGCCTA | CTCATTAGATGTGGGAGATGAGGTTCTCAAGCT |
| | CCATATGCCAATGACTCCACCAAATGTTTTC | CCAACAAACCAGTCCTGACCTCGTTTTGGCAGG |
| | TCTTCTTGGGATCTTCTAAAATTCATCTGAA | AACTCAAAGAGAAGTCAGAAGCTTGCTGAATCA |
| | TACTTATAAGTTATGCAAATTTTGGTTATTA | CCCACACCGGCCGGCCGGCCGAGCATCCTGGCA |
| | ATCTAGGTTGTATTACCTTGGGGGAAGTCAG | AGGCCTGTAATTAGAGCCTCTCTTTCACACCTT |
| | TTAATCTCTTTGAACTCAGTTTCTTTATCTG | GAATCTTGAGGGCCCCACGTCTGAAATGAGGGG |
| | TGAACCTGAAAGAACACCTTCAAACTCCAAG | TGTCCCAGTGCCTGCTGCAAGTTTATGAGCAGC |
| | GGTGGCTGTCAGAATTAACTATAGAGGTGCA | ACACAGACTCCTTTCCTTTGGAACTCAGGGGTG |
| | GGTATCAGATGAAAGCTATAAAACAGTTTAC | CTGCCTGCGTCTGGCTTCTGTGGAGGAGGAAGT |
| | AGATCTTAGATATTATGATGGATGGCTATGA | AATGTGTGTGGATTAGTAAAAGATCATTTTCCT |
| | TACGTTTCTCGAATCACTGCTTGCCAATGAG | GCTGTTTGTCTTGGCCTCCGTGCTTCAGAATTC |
| | CTGTACAATCTTCCTGAAGGGGTCTGCCTTT | AAGCACTTGTACTCTTGACCCTGCAGTGGTGGC |
| | CCAATCTGGGCAGCAACAGTTAATGACGGTG | TGGTTTTGAGTCCACTTCCTGTCTGATCGCTAA |
| | TGCCAGGATATCTGTGTCTCCTTTTATCTGC | ACTGCTCCTTCTCTGAGGACCTTCAGCTGAAGC |
| | TCCAGACTTTAAACACACCCTCTGATTACAT | CACTTACCTGCTAACACTTAATTAATTAATAAT |
| | CACACTATCAATTTGAAAAAGGGCTCAGAGC | TAATATTGTAATTAATTTTTTGTTGCAGGATTG |
| | CAAAATCACCACTGTTAGCGAGTTCTCCAGG | GCAGTGAAACCCAAAACGTCACACATGCTAAGC |
| | GCTGCCTCCTATCCTCTGGAGGTGGGGCTCT | AGGCACGGGGCCATCAAATCATTTTCTTAATTT |
| | CGTCTGCAGAAATAGGCATAAGGGTTTTCTA | TTTACTTTTTTATTTTTTGTGTGTGACAGGGTC |
| | TGGTTTTTGTTTGTTTTAAAGACGAAACATG | TCAAGTAACCCAGGTTGACCTTAAACTTCCTGT |
| | TTTTGGGATCTTTTAAGAATCCTAATCGTTG | GTGGCCAGAATGGCTTTGAATCTCTGGCCCTTC |
| | TGAAAGAAACTGAAGTAAGTTACTGTTCAAG | TTCTCCCTCCCATGGTACTGAGATTACAGGTAT |
| | TGACTCTCATTCTGCTGTGAATAGTTTCTCC | GTACCACCATGCCTGACACCCTGATGCTGTGGT |
| | CACGTGAAGTCAGCTCAAGAGACTGTGAATT | GGACTCAAGGAATGCACATACCTAAGCTTGAAT |
| | GCTTCAGCCTACCTGAGACCTGGTACACAGG | GCTCGCTGTTGAAATACTAGAGACATTTAAAAT |
| | GAGGCTTCCTAGCCACGGAAGAGGAGAGCGT | AATTTGCCAGTTAGGAAAAGCTTTCTATGGCAC |
| | TTGCAGGAGGAGAAGGAGGAGAGAGGGCCCA | ACAGTCCAATTGAATCTTAACACACACACACAC |
| | CGCAGGTGACATTCTGGAAAGGGAATGCTGG | ACACACACACACACACACACACACACACACACA |
| | TGCGAAACTGCCTCACCTACTTTGCTCCTTG | AGACTTAGGTCTTTCAAATTCCAGCTTGGTGGC |
| | GATGTTCAGGAAAAGCCAGCCCCATCCGCCC | TTGTTCCATGTCTTCTTTGGACAAGCCCTCCAG |
| | CAGTCCGAGGGCCTCACTCATGGAACAAATG | CTCTCCTCTCCTCTGCTCTCCTCCTTGGTAACT |
| | AAGCTGAGAAGAGGAGCTTCCTGTTTTCCAG | AAGGGGAGGCCACGCCTACTTTATTGGCATCCT |
| | CTGCTGGGGTCATCATTATCTTCAGGAAGGA | AGAGATGCCAACATTGGCAAAGAGAAGGGACAA |
| | CCCCGAAAAGCATCGTGTGTTGTTGCAAAGG | TTAAATTCATTGAGGCCTGTGTGGTGTGTCAGC |
| | CCTGCCTTATCCTGGCCCCCAGGTCCCTCTC | AACTCTGCCAACCACTTTCTTATCTTGGTATCA |
| | CGCTGGCCCTGTCTACTGGATAAGCTGAGGT | TTTAAATTAGTTTGAACACTTAAAAGGTTGTGT |
| | TGCACGAAGTAGGTCCAGGCCTAATGTGACA | AAATGTGGCTGTCTAGTATTAGAAGCTGTTTTG |
| | GTGAATAATATGGTGTTTGGCCACACAGAGA | TATTATTGTTAGTTGTGTTCCCTCAGGGGAAGT |
| | TGTGTGTAGGTACAAAAACCACCATGCTTTT | GAGCTGCCCTGAGCTCAGTTCTTTATCTGGAAA |
| | GGCGGCAAAGTAAAAAATGAAGATGTCGTCA | CTGGGCCTAATACCTCCAGACTCAAATGACTGT |
| | AACGATCTGAACTCTGATGGAGACTGAGCGA | CACAGGACTTAGCTATGAAGGAAAGGGTTGAGG |
| | GAGACCCTGGCCCAAAACAATCACTCCATGG | CAGAAGTCAGAGCACTTTACAAATATTAGGCGC |
| | CGGATGCGCTCTGGGGTAGACAGCTACTGCT | ACTTACTAATGCTCATGATAAATTCTTCAAATT |
| | CTCAGAGCAGCTGTTTTCAGGCCA | GTTGTGCGATAAAGATCTTGTCAGGGTTTCTCA |
| | (SEQ ID NO: 10) | GGCGGCTATCTTTCCCATCAGAGCTGTCTGTCC |
| | | AAGTTAAAGACAGCTTACTGGAATATTTCTGTA |
| | | TCCTTTTGTCCAATACAGGATTTAAATATACCC |
| | | TGCGATTAGATTGTAATGCCAATAAAAAGAAAA |
| | | GAGGGGATGTCAGAGCATAAGCCCAGGGTGACA |
| | | ACCCTGGGACTGGCATTCTAGATTCTGGGGAGG |
| | | AGACTCTTTCTGGGAAGAGAGGCTCATGGCGTT |
| | | TTGCAGTTTTTGTTTTCTGTTTTAAGACAGGAG |
| | | TTGCTTTGGGGAGCTTTATCTTAAGAATCCGAA |
| | | CGGTTGTGTAGGCAAGCAAGCAAGCAAGGCAGC |
| | | TACTGTTCGGTTGACCTCGTTCTGCTGTGAAGA |
| | | ATTTGCACTGTGTGAAGTGTGTTCAGGAAACCC |
| | | TGAATAGCCTTGGCACACCTCCGACGTGCTGCT |
| | | TCGTGGTAAAGTTTCCTGTCCTCAAAAGAGAAG |
| | | ACATTTAAAGGAAGAGGAGGGACCAAAGAACGG |
| | | GTCACCTAGACAACAGGGATCTGGGCACCTGGT |
| | | AGGAAGGAAACCTTAGCTTATTTACTCCTTGAA |
| | | TGTTGGGAGAGAACAGCCAGGACCCTGCCCTAG |
| | | AGCCTCACTCATGAAAGCTGAATCTGGGACAGT |
| | | GAGTCCTCCCCTCTAACTGCTCCCAGTTCCACT |
| | | GTCTCCAGGGTGGATCCCAAGTGGATGCTGTGT |
| | | ACATGGCCTTCATTCTGGTGCCTAAGCTCCACT |
| | | CTGTGGACCCTGTCACCAAGTTGGTGTGAGGAA |
| | | ATGTAACATTTAATATTATGGGTCTGGGCCACA |
| | | CCAATAAACTACGAGGCATTGTAGTCAAAGCTG |
| | | CTGCCGCCTTTCAGTCACCTGACCTCGGTGGCC |
| | | ATTGAATAAGTGACCTTGGTCTAAAACAATTGC |
| | | TCCAATGTTCTGTTCTGATGCTCTGGGTGGATC |
| | | GCTGCTTGTGTCAGAGCAGATGTTTCCAGGCTG |
| | | TTGCTGGGGCCAATGTCACCATTCCTGTTAGTT |
| | | TCAGATTGTCTATTAGTTCTAGATAGGGTCTCA |
| | | TTATATGAGACACCCCACCCTCCTGCATGGCTC |
| | | AAAAGTTTACTGATTTTTATTCTTTGTGTGTAA |
| | | GTGTCTTGTGTGCACGCACATATATGTGCACCA |
| | | TATGCATTCCTGGTGGTAGGAAGCTAGAAGAGG |
| | | GGCTCAGATTCTCTGGAACTGGAGTTACAGATA |
| | | GTCGTGAGT (SEQ ID NO: 11) |
| D | ATCACGCAGCCCATACCCTGCGGTTCTCCGG | AATCATGCAGCCTGAATGGGCATTTCTCTCCAA |
| | GGACTTATGCATCGGCCCAAGTTGAGGGTTT | GTCGCAGGGTTTGACTGACCATAAACATCATTC |
| | GTCTGAACTGAAACCCGCATCCTAGACCTGG | CTTGCTGTGCTTTTCTGCCCGCTCCCCAAATCG |
| | CTTTCTTCTCCCCAAATCCAAGGGGACACCC | ATGACAGCCCCAAACCAGCAAAGGAAATGAGAA |
| | CGGTGACCCACAAAAGCTTAGAAAATCCAAC | AAGGGACTTAATCCGGACTCTAGTCACTTTAAA |
| | ACGCAGCAAATGAAACGGGGGAAAGGGGCAC | CAGCCTGGTGTGTTTATAAAACCTGTCGTGCAA |
| | CGGCCCTCACTCTGGCCTCTTAGACACACGA | GTCAGAGGGGCATGGTGCATGCAGAAGTCAAAC |
| | TATGAAACCTTCATAAAACCTGTTGTACAAG | TAGTCCATCCCAGTTCCTACTGCAGGGCACGAG |
| | TCAAAGGGGACCACGCTGGGGTAAAAGTCAA | GGAGGGGGCGGCGCGGGTGACAACCACCCTGCC |
| | ACCAGTCCATCCTCGTTCCTCTGCGTACAGA | GCGGTTCCAGTTCCCGGTGGGCTCGCAAAGGCG |
| | GAGAGGGTCCAGCGCGGGCGGCGCCCACTGC | GGATGCCGATGGGAGGCAGATAAGGATGCTGGC |
| | CATCGGGCCGGGGCCGGGGCGCGTGGACAGG | AAACCCCCGCCTCCCCCCCCCCCACCCCCCGCA |
| | AGGGTGCGGATAGAGGCAGATCGGGGGCCCG | TGGTCAAGACTGTCTGTAACCGCCGGGCCGCCT |
| | GTCGCCCCACGTGCGGCCAGACACCCATCCC | GGAGATACTTGCCACCCCCTCGTCCCACAAATC |
| | GGCCGCGCTCTGCCGGCTCTGATCCGGTGCC | TGGCGAGAAAGGGAACAGACCACTTCCTTTACC |
| | AGACAGGAGCGACAGGGGCGAGGTGGGGACC | TGCCCGGGTTTCTCGGAGGAAATGCTCCCACTC |
| | AGCCGCCGACCTCACCTGTTTTGTTTTCTTG | GCGCTTACCTGCTCGGTGGGAGCCGGCTCCAGG |
| | GAGGAAATTCCTCCGCTGGGGGGCCGAGGTG | CTCGCAGCGGCACTCAGAGCTCCTACCCTGAGC |
| | GCACCGCCCGCTCGCCCCCCGCAAGACCCAG | GTAGGTTGGATCAGGCGCCGGCGGTTCACAGCG |
| | CCGGTCCGCGCCCGCTTACCTGCTCTGCGGC | GGAATGGAATCGGGGACAGTGCGGGTGGAGCCC |
| | CGGCGGCCCTGGCGCGGGCTCTGCGCGGGGC | CGGTTTCCACCTGTGGCTTCTTTTAACCGCGCC |
| | GGCGCCCTTCGCTCCGGCTGGGCAGGCAGGT | CCCACCCCGCCTCTGCCTGACGCCGCACGGGAG |
| | CGGGCTCGGGCGCCGCCGGCTGTCGGGCTCT | GGCTGCGGGAGAGGAGCGCGGGCACTCGACGCG |
| | CGTCGGGTTTCGGGTGAAGGCCCCGGCTCCC | CCTTCTGTGGTGCGCACCGCCCTCTCTCCGGGA |
| | ACCTGCTGCGCCTTTTAACCGCGCCCCACCC | CAGAGGAGCGGGGCGGGTCCCCTTCTGTGGAGC |
| | CGCCTCTGCCCTGACGCGGCTCGGGCGGGCT | AAGGGGCAGGGGACCTTCCCTGTTAGGGCCAGG |
| | GCGGGAGGCGAGCGCTGTCACTCGACGAGCC | TCTTAGTGGTACTATATTAGGGCACTCGTTGGG |
| | CCCCGCCCCCACCTACCCGGGGCGCACTAGC | ATCCTTCTTCTGAAGCCAGGGACCACTGCGAGT |
| | CGCTGGGCGCGGACCGTCCCCCTGAGGAGCA | GTCCCCTAGGAGAGACTCCAGGTGTAGGCTGGT |
| | AGGAGTGCAGGACCGGGGCTGTCCCTCCGGG | CTTCCCTTGGGTTGGGGACAGAAGGCTTGTCCC |
| | GCCGGATGCGCAGAGCGGGGACCTTTTTCCC | TTCTTGTGGATGTGGGTGGAGCGTGGACCGCGA |
| | GTGGCGGGGGCGCAGGGTGGGGGACCCCTAA | TGGGCAAGCTCAGCCAGATCCCATCAAGGACAG |
| | GAAGTGCACAGTGCGCGGGGCCCTCTTTCCG | GGAAAAGTTGCCCGCTGGGGCCTTGCTGGGGCT |
| | GCCCTTGGAGGGAACGGGGTACCGGGGATGC | GGACACTGGAGGGCCCTTAATGAAGTGAGGGCT |
| | AGGGGGTAGGGCTCTCCCTCGGGAGCGCAGA | ATCCAGAGTACGGGGAACAGGCTTGTGGACCCA |
| | GGGCGGGCCCAGCCCCCTCTGCACGGGTGCA | GCTAGTAGTGAGTCTCTCCTGTTGGTCATCCTG |
| | GGTGTGGGGCGCCTGCTCAGGCCCTCGAGGG | GTAGGAAGACAACTGGTTTGTTTTCATCCTTTC |
| | AACTCTTCCTCCCTAGTGCACCCGTGGGGAG | TAGACCCTTTGGGCACCCTCTCCTCTAGAGCAG |
| | CAGTGTGAGGGGCAGGCTGTGTTTTTGCCAG | CCTGGAGGTTCTTTATTCCTTAATGACCACTTA |
| | GACACATCCTCAGTCTTTCTGGGTGATCCAG | GGAGTCTCAAAGGTTTGTTTTTATTAGTCATCT |
| | CCTTCTCATAGCCCGCGGGGTGCACAGACCT | GAATCCCTTCCTGCATTGTCCAGGGAAGGGGAG |
| | CTCCTATAGGAGCCTGGAGGTTCTTTATTAA | TGGACTTCCATCTTGAGAGATCCCACTGTGTCT |
| | TTAATGACCACTTAGAGGAGGTACAGGGGTT | GCTGTCACATCAAGGGCAGGGTAAGGTCAAGGC |
| | GTTTTTATTAATTACCTCCATCCTTTGAAGA | AAGCATAGAGGGTGGTACAGGGGGTCCTGGGCT |
| | CTCCTCCGGGGAAGCGGAGCAGGCCTTCCTC | GGAAATGTTGGAAGCCATGTAAGGACCTAGTTT |
| | GGGACAGTGCACCAGGAGAGACCACATTGCC | TACAGGGCCTGCCCTGTGCTACTTCAGACAAGA |
| | TCCCCGCTTTTCAGTCAAGACTAGAAAGCTC | CTTGTAACATGTGTAACTTGGTTATTTTACAAA |
| | AGGGCCAGTACAGGGAGTGGTGCAAGGGCTG | ATTGGCTGGCAGGTATGTTCTTACCTGTTGGGT |
| | GTGGGGTGGAAACGTTGGAAGCTATTTAGGC | CATATTCTCACTTTAGCTACATTCTACCTGTTG |
| | ACCTGGCTTTACAGGTTCAAACCTGTCACGC | GTTCACGTTCTCTCACAAAACGAGAGTAATAGT |
| | ATCGGACAAAAGATGTGTGACTTGCTTATTC | GCTTCCTAAAATGTCTCTCCCAGGTCATGGAGG |
| | TACAAAACTGTTCGGTAATTAAACGTCCCCA | TTGAGTCAACGCTTTATAAAAACCCACCTTAAT |
| | CCTAAACCATATGCCACTTGTTGGGTCATAT | AAAATACTTGAACCAGAGTTCTCGGAATTGGAC |
| | TCTCCCACGAAACAATTAAGATGTCTGTTAA | CC (SEQ ID NO: 13) |
| | AGGTCATGGAATTTGAGCCAAGACTTCATAA | |
| | AAATCCGCTTTCCAAAATATTTTATTTGAGG | |
| | AGAACAAGGTTCTTAAAGAATTTGCCCAAGT | |
| | C (SEQ ID NO: 12) | |
| E | TAAAAGTGAGCAAACAGCTTGAACCAATCTA | AGGAGGTGTGTCTTCCTGGAGGAAATATGTCAC |
| | AACAGCTTATTTATTTGAGGTAATAAACTTT | AAGGGTGGGCTTTGAGCATTTAAAAATTTACCC |
| | TCCTTCTTCCTGAGTTTTCCTAAATTCTTCT | CCTTTCCAGGTTTTTCTCTCTGCTTCCTGCTTA |
| | CTATCATGAAAATAGCATTAATAGCTAAAAT | TGGTTCAAGATACAAACTCTCAGCTTCCAGCTT |
| | TTTAAGTGTTTAGAGGTTTTGCCTTTCAAAT | CAGCCCCTCTGCTCTCAGAGATGCTCATCTCTC |
| | CCAGTAAGTCTCCAGAGTCAACAGGTGCTAC | TGGAACCATGGGTCCAAATAAACTCTTTGTTCT |
| | AAGATGCTACTGGCAGTAACAGTGCTTCTCC | ATAAGTTACCATGGTCACGGTGCTTTACCACAG |
| | AGGATTGTGGTAGGTGGTGTCTAAGGGTCTT | CAACAGCAAAGTAGCTAATATAATCTTTTCAAG |
| | TTCAGCTTGAAGGTTCTGTTTCCCAGTTCTG | GCCACGAAAAAGAGAAAGGCAAACCAAGAGTTT |
| | TCTCACTTAAGATCAGATCTTGGTGAGTATA | GGCTGACCAAATCAGCTGAGAACACAAACCTTC |
| | TTGGCAAACCATTTCATTATTTAAATTTGTA | CCATCCTAAATTCCCCAATGTTCTTTTATTTTT |
| | AAATACAGGCTTTAGGCCGGGCGCGGTGGCT | CATCATGCAAATAGCCACTGATATTTAAATTAT |
| | CACACCTGTAATCCCAGCACTTTGGGAGGCC | ATTAATGTGCTCATTATGGCAGTTTCATATATT |
| | CAGGCGGGCAGATCACCTGAGGTTGGGAGTT | TATATATTGTACTTTGAACATATTCACACACCT |
| | TGAGACCAGCCTGACCAACATGGTGAAACTA | CCAAATACCCTCTTCTGTCCCCCACATTTTAAG |
| | CGTCTCTACTGAAAATACAAACTTAGCCAGG | ACTGGAAGTCTCGTTTTTTCAAATCCATTATTA |
| | CTTGGTGGCACATGCCTGTAATCCCAGCTAC | GGTCCTTAGGGTCAATGGGGTCATATGATGGTG |
| | TCGAGAGGCTGAGGCAGGAGAATCGCTTGAA | TCTGTGGTTCTAATTAGTGGCCAGCTGGATACC |
| | CCCGAGAGGCGGAGGTTGCTGTGAGCTAAGA | TGCAGAATCAATGACTAGTGGGTAAAAAGTGAG |
| | TTGTGCCATTGCACTCCAGCTTGGGCAACAA | CAGTCAGGGTCAGCAGCTCACAAAGCGTCAGTG |
| | GAATGAAACTCCATCTCAAAAAAAAAAAAAC | AGAGGCGGACAAAGAGAGCTTTCAGCAACCCCT |
| | AACAACAACAACAAAAACAGGCTTTAATTGT | AACTGGGTGGGCAGCATGTGAGCCAAGTGTGAG |
| | ATTTCATACTCTTTAACTAACTAGATATTAA | TCCCTCCTTTTTGGACCTGGGAGACCAGCAGAG |
| | CTATAAAATATTAACAATTTCAAATTTTTGT | TGTGCAGGCCCTCCGTTGGCTTGGCCCAGGTGA |
| | TAAAGGAATACATTTACACAGCTTAAAAATT | TAAGCTGACCTCAGCAGGAATTACCTCAGTCTT |
| | CAAGTGGAACTAAAAGGTTTACAAGGCAATA | AGTCCAGCTCCTGATGTAAGTCTCACTCAAAAC |
| | TTTCAGTCCTCTGCCCCATTCTCTGCTCCTC | AAAACAAACAAGCCTAGACAAAACCAGCTTGTT |
| | CCACCCTGTATGCTGTCCCAGAGGCAACCAA | GTCTTTTTTCTGTTGTGGGAACTGCTCCCACTC |
| | CGCCTTTCATTTTTTAGAGCTCTTCTGACGT | AGGAATTTCTCAGTGGCCCCCTCAAGGAAGTTT |
| | TTACCTTTATGTTTCCAAATAATGTGCTTAT | GCTTCTTCTCTGCTTCCTTCCACACATCTGTGT |
| | TATGCCATTTACTGATTGCTGGACTTTAGAC | CTTTCTGGTTGGAGACCATGGACTTGAGAGTTC |
| | CTGTTGACTTTTTCTGCTATGGTAGTGGAGG | AAGTTGAGCTTCCACTACCCTAAGTGCCTGGGT |
| | CTTTAGCTCTGACCTGAGCCCCACTGCTCCT | CAAGCACACCTGCGCTGAGAAGGGTCCTGCCAG |
| | GCTCCACCCACACCTCTTCCCTCACCCTCAT | TCTCAAAACTGCATCACTAGATCAGCAGTATAC |
| | GACATGATCATGGCTCATACTCTGGTCAAAT | TCTCTCACTTAAGCATGGAGTGGGGAGGTGCCT |
| | ACATATTGTTATTTATATTATTTTGACTGCG | TTGTATGTCTTAGCAATAGTCATCTACGTGATT |
| | AGCATAATGACGTCTGGACCAAGTTGTATTC | TTGAGGTCATTTTACTTTTAAAGTATATAATCT |
| | TATGTTACATTTTCTTTTGGTTGCAATTGCC | TCAAACCAAATTCAAAGACTAGGCAAAATTTTT |
| | TCCCTTCCCTGAGAGTGAACCATGACTGGGG | AAATTAGCTTTTAAAAAATGAGCTGGTTTGCTT |
| | TTTTCATTTGCTTGGCTTTCTATGTGTCTAT | ACTTCCCTGATCTTAATTCCTATAGGCAGTATT |
| | TGTTCGGCTTTTCCTACTCTTCCAACAAATC | GTGAGGTAACTTATTTAGGTTTAGGGATGATAG |
| | TGTCATATGCCCGGAAACAATTTTTTCAAGT | AGAAATAATGTCTTAGGGTTTTACTCCTGTGAA |
| | TCCCAGACATGGTTCCGCACAGTCCATCTAT | CAGACACTATGACCAAGGCAACACTTATAAAGA |
| | TCCATCTGTTTCTTTCCCTTTTCCCGGGGGC | CAATGTTTAATTGGGGCTGGCTTACAGGTTCAG |
| | TGTGGTCTGGGCAGGGTGCTCTGGCCCTCTG | TTGTTCAGTCCATTATCAAGGCAGGAACATGGC |
| | CCCAGTGGTCCCCTGGGCTCCCCTTGCCTTT | AGTGTCTAGGCAGGTATGGTGCAGGAGGAGCTG |
| | CCCCTGGGCCAGAGCTTGTGCTTTCTGGAGT | AGAGTTCTACAGCTTCATCTGAAGGAAGCTACG |
| | CCGTGTCTTCCTGTCTTGGTCTCTACCTTCA | AGAATCCTGGCTTCTAGGAAGCTAGGATGAGGA |
| | TTTTGCTGAAGCACACACCTTCCAGGAACTT | TCTTAAAGCCCACGCTCACAGTGACACACTTCT |
| | CCTCAGGAGGGGAATGTGGAACTAAACTTCT | TCCAACAAGGCCACACCTCCAAATAGTGCCACT |
| | ATGCACATAAAGTCTTCATATCACCCTCAAA | CCTTGGGCCAAGCATATTCAAATCACTATGGGT |
| | CCCGATCTGTCTCCCCGCCTCCAATGTACTT | ACTCTTAAAAGAATGCATGTTTTAGCTTTAAAC |
| | TCCTTTCCTCTCTTATTTTCTCTGTTTTTAT | ATTGTTCATTTATCCGTGTAACAGACTGGTTTG |
| | GAACTTACACCTTTTTTCTTCACTATTGTGT | AGATCTCTCAGCAAAGGGAGTTATCCTTATACA |
| | AATTGGCATTTAAGATGGGAGTAGAGATAAA | GGGACTCTTTTCATTCTTTTTCTTAGTGCATAT |
| | TGCACCTGTGTAGGCTCATACTAACCACACG | TCATTGTAGATAGTGCTGAGTTGTATAAAGGCT |
| | CCTCAGTGCATGGGTGTTTATCAGACTTCTC | TTATCTATCTATCTATCTATCTATCTATCTACA |
| | TCAATCAAGAGCTGCGCTGAGTACTTGTGAA | TCCCAAATGTTGCCCCCCTCCCCGTACCCCCTC |
| | GGCCCTGCAGGGCTGGTGCTGAGTAAGTTCA | AAAGAGTTCTTTCTCCCACCCCCATTCTCTTTG |
| | GGATTGGGCACCTCTGAGGGGTGAGGAAATG | CCTTTAAGAGGCAACCTCCTCTTATATCTCCCC |
| | GAGGTTCAGAGACGAGAAGGAACTTCCCCAA | AACCTGATGCATCAAATCTCTGCAGGATTAGGC |
| | GGCCACATGGTTAATGATTGGAAGATCTGAG | CTCAGGCCAGCCCATGTATGCTCTTTGGTTGGT |
| | ATTCTAAACCAAACCTGAGTCGATCACTTCC | GACTCAGTCTCTGGAAGCTCCCAGGGGTCCAGG |
| | CTTTCTGTCCACTGCACTGATAACTGAAGCC | TTAGTTGACACTGTTGGTTTTCTTGTGGGGTTG |
| | CAAGGGCTGAGGCCACACCTCAGCGTGTGAG | CCATCTGCTTGAGGGCCTTCAATCCTTCCCCTA |
| | GATCAGCAGAGGAGACCCTGCTGGCTGCGGG | ACTCTCCCACAGGGGTTCCCAACCTCCAGTCAG |
| | ATGTGGATAGGCTTTGAGGAAGAGGAAAAGC | TCCAGTGTTTATCTATGGGTATCTGGATATCCC |
| | ACAGGCAAAATGTCAAAGATAAGTGGGAATG | CCTCTGTCTCATCAGCTGCTGGGTACAGCCTCT |
| | AGGTTCCCTGGAGCATGAGTCGCAGGTGCTC | CAGAGGCCTGCTATGCTAGGCTCCTGTCTGCAA |
| | AGGAAGGTGCTGGCAGCTCTAGAGAAGGCCA | GCACAACATAGTATCATCAATGGTGTGAGTGAT |
| | GAGAGAAGCACCCAGTGGTGGGAGCCACAGC | GGGTGCCTGCCCATGGGATGGGTCTCAAAACGA |
| | CCCAAGACACAGGCTAAAGCCCCAGCCCAGG | TCTGATCACTGGTCAGCCATTCCTTCAGTCTTT |
| | GTGGGTGAGCTCCACCCTGTCACCTATGGGG | GCTCCATCTTTGTCCCTGCCTTTCTTTTAGACA |
| | TTGCATGCAAGTGGTTCCTCTAAGCATTGGC | AGATCAATTTGGGGTCAAATTATAAAGGCATTT |
| | TTCATCTGGGAGGCGGGGGTGACATCGCTTC | TCATGTTAAGTGTATAATGTATTTTGACCATGT |
| | TTTGAGCCTTATTTGGAGGACTAAACAACAC | TTCCCCATATCCTCCTACCCTCCCATTTGCCCT |
| | ATGCATTTTGTCATTAGGCTGGTGCAAAAGT | CCCCCTTTCTCATTAGTATTCTTTGTTCTAGAC |
| | AATTGTGGTTTTTTTCTATTACTTTTAATGG | AAATTTACTCTACTTTTATGGCATATGACACAT |
| | TAAAAACCGCAATTAGTTTTGCAGCAACATA | ACATGATTTAATGAAACATAAAATGGAGAATCT |
| | CTAACTTTAAAGTTCTTAATACATATGAGAT | ACAGACAAAAGAAAGCATGAAATATTTGGCTGA |
| | ATTATTTCTATCAGCTTAGAAGGATCCATTA | AGCTGACTCAACTCATTTAATATGACAACCTCC |
| | TGATTGTAGAAGACCTGGGATGCCAGTCTGA | ATTTCCCTACAAATAAGAGAATCTCATTCTTTA |
| | GGAACTCTTCTTTTCTTAAGCAAAGGAGAAA | TTGCAGACTAAAATTCCACAGGTGTATATACCA |
| | CAAAATAATTCTGATGGGGGAGTGACTGACC | CATTTCTTTCCCTATCCCTCTGTCTTTGGACAC |
| | CCAGTCTGGCTCACCGGCGGCTGTGAAGTCC | CTAGGCAGGTTCCACCGTGTAGCTATTGTGAGT |
| | TGAGTGTCCTCTGGCAGCTGCCTTTGAAAGC | AATGCTGTAGTCAACATTGACATGCAAGTGTCT |
| | GCAGTGGTGTCCGGGGCTCGCCACTGAATAG | CTGTGACATGTTGACACAGAGTTCTCTGGATAA |
| | CGTTTGTTCTCAGAAGGGAGCCCGGTGGAAA | ACACATAGGAGTGTCGTAGCTGAATGGCAGTCG |
| | ATTTGAAGCTGCAGTTAGGAACTGTGTGTAT | ATTGAGAAAACAAATAATAAAAGGGTTGGTGAG |
| | GGCCTTGGAAACTGAAGATGTTCCTTTAAAA | CAGGTGGGAAAAGGAAACTTTGAACGCATTGCT |
| | GAAAAATCACAGTGTTTTTAAAACTCAGATG | GGTGAGAAGGAAAGTCAGTCTAGCTGCTATGGA |
| | ACAGCTTTGACCATTATCTGCTTTCCTCTCC | AATCAGGGCGAGGGTTCCTCAGGCCCTAAAACC |
| | TGCCAGCTCTAGAGTTTTCTTGGGATGTTAT | AGAACTGCCTTATGACCCAGGCAGTCTTGACAG |
| | CAAGGATGATATCACAACAATGCCCACTTCT | CTGTTGTTGTCTGTGCTTAAGTTCTTGACTCTG |
| | GTTTTGTTTTTAACCTGAATGACAAATTACC | TCAGACATAGAGAAACCAGATCTCAGGCTAGAA |
| | AATCAGCAGATGTAGGCCATCCAGGGAAGTT | GTTCCTTCTTTCTCCATGTTCCCTTAACCACCC |
| | TCTTTTAAATGCTGGACTTTTGCAAAAATGT | TCTTCTCTCCTGCCTCAGCCTTGTAGAAGTGTG |
| | AGAGCCTTGGTGGCAATTGTGATTCTTTTTT | CCTTCCATTAGGCACCTAAGAAGAGGAACTTGA |
| | TTTTCTTTTCTTTTCCCCAATGAAGGTACTT | CAGTCAGCTGCCACCTTCTAGTGACTGGAAGAA |
| | TTTTTTATGTCCAGTTTTGGAAGGCTCCTGA | CCAAATATTCTGGATCTGAATAAAAGATTTTAC |
| | AGATTGTTTGAGAACTTGACTGCTGTGTCAG | ATTCTGCTTTGTGGCTCACAGGAGACTCAGTGA |
| | GGCAGTGCTGACACTCTCTGTTGCCAACTGT | CAGGCCCACCTAAGCACACACAGAACAGTAGAG |
| | TATTCATTATTCCAAAAAATCAGAGAAGCAA | CGACAGGTTGAAACAGCTTCCAGGAGGAGTGGG |
| | AAACGACCCCTCCAAACAACTCCAAGACAAA | GGGAGGACGGGCTGAGGAAGTGGGATGTGTAAT |
| | CTCCAAGCAAAACAACAACACACACACAAAC | TCCAGTAGAGAAAGTCATTGGAGGTACGGAAGG |
| | CCACAATTTTCCTTTGGTTGCTTCTGAGAAG | TGCTGGCAACCCTGAGAAACAGCAGCTGATCCA |
| | GAGTTTTAATGGTATAGTAAATACAGCATTT | CCAGCTGCAGGGCCAGGCCTCTGGATGCAACAG |
| | ATCGGATGATTTTTGCTGCCATTGATATGTT | CCAAGTCAGAGCCCAGCTGGGCCTGGCTGTGTT |
| | TCTCTTCTTG | CCACCTGCTCCCTGGGTGGCCCCAGGCAAGTGA |
| | (SEQ ID NO: 14) | CTCCCCTGAGAACTGGCTTCAGTAGTGAGAAGA |
| | | GGGGTGGGGTGACAATAGCCTCTTTACAGGGTT |
| | | ACCTAGAGGACTAAATAATGCACATACGCATAC |
| | | ACACACACAGACATGCACACATAGACGCACACA |
| | | TAGACACATAGACACAGACACACACACAGAAAC |
| | | AGACACTGACACACACATACACATACACAAAGA |
| | | CACACAGAAACAGACACATACATATATGTATAC |
| | | ACACAGAGATATACAAATATACATACACACATG |
| | | GACACAAACACACACATACAGAAACAGACACAC |
| | | AGACACACACACCAACATATAATACACACCCAT |
| | | ATAACACACACATATAACACACACACACAGGCA |
| | | AACACATGGGTTTATGGGCTCTGCAGTACAATA |
| | | AGGCTTTATTTTCATCAGCTTAGTCAGCAGTAG |
| | | CCTACAAATATTAGTGTTCAAAAGTATTTTCTA |
| | | GGCAAGGGAGAGACAGAAAGTGGTTGTGGTGGG |
| | | GAGTGAGGCTGGTGACTGTGAGTGGGCAGTGTC |
| | | TAGTGTCTGGGGACAGCTGAGATTGGCAGCCCA |
| | | CTGGCCACTGACTAGAGTTGCTTCCCACAAGTG |
| | | AGTCCAGTGGAAATTTTTAGTTTGCTCTTAGAA |
| | | ACTGTGCCTTCAGCCTTGGAAACTGAAGATGTT |
| | | TCTTTAAAAGAAAAATCGTGCTTTTTGAAACTC |
| | | AAATGAGAGCATTGCCTGCGGTCTGCTTTTCTC |
| | | TCTCTCTCTCTCACCAGTTTTCCTGGGATGTTA |
| | | TCAGGGCCAATCATCAGAACAATGCTCACTTCT |
| | | ATCTTGTGTCTAACCTGGATGACAAATGGCCAG |
| | | TCAGCCGATGTAGGTCACGCAAGGAAGTCTGTC |
| | | TTTCGGGTTGGACTGAGGTAGCCGCAGTGCGAT |
| | | GGCTGCTTTGTTGTTTCTTTCCCTTTTCTTGTC |
| | | CCAACTAAAAGCGCTTCTGGTCTGGGAGTAGGG |
| | | GCGACTGAAGGCTGTTTGAGAACTTGACTGCTG |
| | | GGCCCCTCTAACATTTTCTGTTGCCAACAGCTT |
| | | ACTCCTTTTGCTAAAAAAAAAAAAAAAAAAAAA |
| | | AAAAGCAAACAAGCCCAAACTACTTCTTCAAAC |
| | | AATTCTAAGACACCACACAAACAGAACAGACTG |
| | | AAGCCCCAGTAACCCAGCTTTCCCAGGGATGTT |
| | | TGTGAGAACCAGGGTAGTTTTTGATCACTACTA |
| | | AATTCTACTTAAACATTTTTAAAGGATTTCTTT |
| | | TTCTTCTCGTTTTTAAATTTGTTCTTCGAATAC |
| | | AATGTATTTTTGATCATATGTGCACCCCTCCCC |
| | | CAACCCCTCCTTCTATCAAGCCAACCTGGTGTT |
| | | CCCTCCCCTCCCCTCTCCCTCCTCCTCTCCCTC |
| | | CCCTCCCTCTCTCCTTCCCTTTCCCTCATCTCC |
| | | CCCTCCCCTTCCCCTCATTTCCCCCTCCCCTTC |
| | | CC (SEQ ID NO: 15) |
| F | GTTTTAATGGTATAGTAAATACAGCATTTAT | GGGTAGTTTTTGATCACTACTAAATTCTACTTA |
| | CGGATGATTTTTGCTGCCATTGATATGTTTC | AACATTTTTAAAGGATTTCTTTTTCTTCTCGTT |
| | TCTTCTTGAAAGAGGAATTCAAATGACAATG | TTTAAATTTGTTCTTCGAATACAATGTATTTTT |
| | AACATTTTTGGGGTCCTCTTTTATGGAGTTT | GATCATATGTGCACCCCTCCCCCAACCCCTCCT |
| | GATTTTCAGGGGATTGTCAGGCATGTCGTCT | TCTATCAAGCCAACCTGGTGTTCCCTCCCCTCC |
| | CCGGGTTCCCATGCTGCACAGTCCCAGCACT | CCTCTCCCTCCTCCTCTCCCTCCCCTCCCTCTC |
| | CTCTGTGGCTCAGCCTTCCCGTCCCTTGCCC | TCCTTCCCTTTCCCTCATCTCCCCCTCCCCTTC |
| | TCTGAATACCTTGCCGTTGACTGAATGGTCA | CCCTCATTTCCCCCTCCCCTTCCCCTCCCTCCT |
| | TCGTTAGCACAGGTCATCACAATACATGACT | CCTTCCCCTCCCTTTCTCTCCCCTCCTTTACCT |
| | CCTGGGCAGGAGGAACAGAGGAGCGGAGGTT | CCCCTCTCTTCCCCTTCCCCCTCCCTCCCTCCC |
| | GTGCCATGCATTTAAAACCCAGTTAGCATCC | TTCCTCCTTCTTCTGGAGGTTATGGTAGCACTA |
| | CAGTGGGTCTTCCAAGGCCGAAGATGGCAAA | GGAGTCAAATCCAGAGCCTGACACTCAACTGCT |
| | ACGTTTTTATTTTACTTTGTTGAAATCATCT | GATTGAACCCCTGACCCTTCTTATTTTTTCTGT |
| | GTTTCCCTCCAAATGGTGGGCTGTTTGGGCA | CCATGTTTATTTTCTTGAAGGAGGAATTACATA |
| | CAAGGTCATGTTGTCTTCAATTTCATAGCCC | AAAAATGAGCCTTTCGGAGGTCTTCCTTCCTTG |
| | CGGTACCCAGCAAGGATGGCTGCCCATAGGC | AGTCTGCTGTTAGGGATGAGTCCCGTTTGAATT |
| | TCTATTAAGATGCCGAGTGCATCCGTGGCAC | TCTGTCCATGGCAGGGTCTAGCGCCGATTTCTC |
| | GGCCAGGAGGAGTGTGCTGTGGTCAGCCTTC | TCTGATCCCCAGAACCTCACCCTGATGAGGTTT |
| | CAGAAGGAATCAATCTCCTGGGAGAAGTGGA | GTGCGATGGGTGACACTAAACAGTGTTTTCTAC |
| | GAAGTTGGCCTGCAGCAGGGGCCTCGAGAAT | TAAACAGTGGGCTTTGTGGGGACAGGGTGACAC |
| | GGCGGGTCTCATCCACCACCAGCAGGCTCGT | TGTCTTCCACTTGCTCTGAGTTCCCCGCAGGCA |
| | CTGTTGCCCAGCAGTGTGATCCTAGCTGAGG | TCACCCCCTTCCTCCCCACTGGTGCCCCACTCT |
| | TTTATTCTCTTTCCCTCATTAGACTGCAGTC | CTCTATCTGGGTAGGTTGCAGGCCCCCTCACAG |
| | TCCTGAAAGGCAGGGTGTGCACCTGACTTGT | TTCTACCTGGAACGTGCTGTGGTCAGCGCAGGC |
| | CTTTTTGTCCCTTCATCCTGCGCCCTGCACG | AGGAGCTGGCTGGCCTTTGTAAGACTGGCCAAC |
| | GTTTGATCAGTAAATGGTGGCTGAGAGACAA | TAGAGCGATGCAAAGCCGGCCTGGCACCAACCC |
| | GGGAGTGGGAAGGAAGGAGGTCAGGAGGGGA | GGGCTGCTCTGCAGAAAGCTAGCTGATTTCCAG |
| | GAGAGGTCTGAGTGCTTGAAAGAGTCCCTCC | CCTGAGCAGGTGCCTGTGACTCCAGGGGCAGGG |
| | TCTGCTTCAGGGGCTTGTTCTGGGGTTTTCT | TCTCTGTCAGACGCACCTCTATCCATCCTTCAT |
| | GGATCTTCAGTACTTGCGGGTAGGATCTGAG | CTTATCCCTATGTTCTGACTGTTAAATGGCAAC |
| | CTCTCCCGGCCCCTGGTGGTTGTTGGCCAGG | TGAGTGAGGAGGGGAAGGAAGGCAGAGGAGGGG |
| | CCTGGCCAGCTTCCAGCAGCACAGGTCATCA | TCTGAGAGGGATTTGAGTGTTCCCAGGCCCTTG |
| | TAATATATGACTCCTGGACAGGAGGAACAGA | CAGAGGCTGTCCCGGGTCTGGAGGGCTTCAGCC |
| | GGAGCGGAGGTCGTGCCATGCATTTAAAACC | AGGGTGTCCTATGTAACACAGGATCCTCAGATA |
| | CAGTTAGCATCCCACTGGGTCTTCCAAGGCG | GCAGGTACTGTTAAAGAGGAGGCCATCACACCT |
| | GAAGATGGCAAAACGTTTTTATTTTACTTTG | GTGCATTTGAGACCATGCCAAAGCAAAAGGTGT |
| | TTGAAATGCAGGTTGTTCCTTTTTTTTTAAC | CAACACCCGCATTTTACTGCATGGAAATGTAGT |
| | CAACTTTTATGTTCCAAGGCTAAAACATAGC | TCGTTCCTTTTCAACCTTTTGTATCGTGGGGCT |
| | ATAAAACAATTTGAAAAAGTCGGTTTCAATG | GAAGAGATGATGTGAAAGGACTTTAAAAACTCC |
| | TTTCCCATTGTTCACTGAGAGAGGGTCACAC | ACTAGGCTTCTCTGCTTTGTTCACTGTAGAAGG |
| | AGGGTGCAAGGCAACAGAGGACACCATTGCT | TCACAGGGAGTTCAAGAAAACAGGCTAGGGATA |
| | TACGTAGTACCTCGTGAGCTGCACTGCGAGA | GGAGGATGCTCATGTGCTTCTCTTGTGAGCGGT |
| | GGCCTTTCAAAGGAAGGTTTTATTTAGGAAG | GGCAGGGCCAGCTCCGTCTCAAAGCAGGCTTTA |
| | CAAGGAATGATTAAAAACTGATGGCTCTAAT | TCTAGAAACTGGTGAGGTGGCAGGAGCTTAGGA |
| | CAAATGAGATTTAAAATTTTCCATTAAACCT | GGAGGGAGAAATTGATTTAAATATTTTCATTAA |
| | TCATAGTTAGGCTGCATGCAGTGGCTCATGC | ACACTCCCTCACTGATGGTAATTTCACTTGCTC |
| | TTGTAACTCCAGCACTTTGGGAGGCTGAGAT | TCTCCCTCTTAGCCCCCCACACTTCAGAACAGG |
| | GGGAGGATCACTTGAGGCCAGGAGGTTGAGG | AGAGAGAGGATACTCGCATACACACACATTTAA |
| | CTGCAGTGAGCTGTGACTGGGGCACTGCACT | GTGCAGGCACACACATAGATATGTATTTCTAAA |
| | TCAGTCTGAGTGACAGAGGGAGACTGTATCT | CCATTTTTCCTGTGAATACAATGATGTGCTCCG |
| | CAAAAAATAAAAAAAATTAAAAATTAAAAGA | ATATATACTTAAGCCAGTCTTACTATTAAACCA |
| | AATAAACCTTTAACATTGGGTGTAATTTTAC | TCTCTTCTAAAAAATATGATCAAAACACAGTTG |
| | TTTCCATCTACTCCTTCTTCCTCACCTGCAA | TTCTAAAAGCAAACTCTAAAAGACTGACCTAGT |
| | CGTTCAAGAGCAGGAGGGAAGATGTGAACAC | CTCTGACAATGAGTTTGAAAAAGTGCAGCTCTT |
| | ACATTTGTGTGTGTGTGTAAACATGCTCATG | GGTGTTGTCTGCAAACCCAACACTATTTGTTGA |
| | TGTTTCTAAATTATCAAGTCAGGATAAGAAC | CTTGACAGGCAAGACAGACAAACCCTCAAAGTT |
| | TTCTACTGTGAAATACAGATATACAACAATA | AATGGTTTCTCTATTCGTTTACTCTGTAAGTGC |
| | TGTCCCAAGCTATGTTTAATGCACTTTTATT | TCTCTGCATTCAAGCGAGATACTGCATTGGCTG |
| | ATCCTGCTAGTTCTTCTAAATATGATCATTA | ACACATTAAATATGCTGAGACTCTTCCAGAACG |
| | TACAATAGTTCTTTTTTTTTTTTTTTTTGAG | CAGCAGGCAGACAACCCACGGTCAACAGTGGGG |
| | ATGGAGTCTTGCTCTGTCACCTAGGCTGGAG | GAATGGTATTTGTCTGGCTTAGTTATCTCCAAA |
| | TGCAGTAGCGCAATCTCGGCTCACTGCAACC | TGTCTAGAGAGAGAATAATAGTATATAATGGTG |
| | TCCGCCCCCCAGATTCAAGCAATTATCCTGA | CATGGAAAACACCCATGAGCCTTGGTGTGTTAT |
| | CTCAGCCTCCCGAGTAGCTGGGACTACAGGC | TAGTAGTAGTTACTTTATAGTGGGTAATGACAA |
| | GCGTGCCACCACACCCAGCTAATTTTTGTAT | AATAAAGGTAGCTTCCAGTTTCTGAAGGTTTAC |
| | TTTTAGTAGAGACGGGGGTCTTGCCTCGTGG | TATGTGTGGATGTAACCCTTGCTAATCACCACC |
| | GCCAGTTTGGTCTCGAACTCCTGACCTCAGG | TTAGTTAATCCAAACAACAGTCCCATGAAGTAT |
| | TGATCCACCCACCTTGGCCTCCCAAAGTGCT | GACTATTATTATCCCCATTTTACAGACAAACAA |
| | AGGATTACAGGTGTGAGCCACTGTGCCCGGC | AATGAGGACTACAGAGGTTAATAACTTGCCCCA |
| | CCATTATACAATAGTTCTACAAAGAAAATTT | AGTCATGGTACCAAAGGGTTTGGGAGCCATTAT |
| | AAGAGCAAGCTCTGGCTTAGTCTTTGAAAAA | TTCAGTCAAATTCTAACCAAGTGTGCTTAGCCA |
| | CAAGTTTGGAATTTCCTATACGAGTGGATAA | TCGTGCCAGAGGTTCCAAGGAAGGAGTTTGCTT |
| | AATGTCAGCTCTTGGTATTGTCCTTAAGACA | GTTTGTTTTATTTATATCACTTGATGAAATAAA |
| | CAGTACATGGTATTTACTCTCTTTTTATAGG | ACTACCATTCCCATTACATATAAAACCTCCTAT |
| | GTAAAGATAGATAAATCCCCAAAGGCCTTGG | AGATGCCTCCTTAGCATGCTGTGTGATTCCACT |
| | CATTTAGGAAACAATCATGCTTTATCTATTA | AAGCTGTTGATAGACACAGTCCTCGGGGCTGGG |
| | ACTTACTCTTTAAGCTCTGTCATTTTTTGCG | GGTGTGGGTCATTTGTTAGCATGCATGAGGTCT |
| | TCTGAGTGAGACACTCTATTTACTGAGCCAC | TGGGTTTGATCCCCAGCACTGATAAAGCTGGCA |
| | AGACCACCTGCTAGATAAGCAGAGACTCTTC | TGGTGATGTATGCCTGTCACCCCAGGACTTCAG |
| | CAGGGCACACAGCCTGGAGAAAAAACGCCTG | AGATGGAGGAAGCCATTCAGTGCCATCACCAGC |
| | TTTAACTGTCCCCAAATGTCTAACTAAGAAT | TACATAATGAGTAAGAAAGAGACCAGCCTGGAA |
| | ATTAGTGGGCCAGGCGCAGTGGCTCACGCCT | CACATGGCATTTTATCTTAAAAAAAAAAAAAGA |
| | GTAATCCCAGCACTTTGGGAGGCCGAGGCGG | CATTCGTTTTGACATGTATATTTTTTGCTTTTG |
| | GCGGATCATGAGGTCAGGAGATCGAGACCAT | TAAATTTTCAAGGGAATGTTTCACCCAGAAGCT |
| | CCTGGCTAACACAGTGAAACCCCATCTCTAC | TTGCACTGCTGATGGTACACGTCTGAAATGTCA |
| | TGAAAATACAAAAAAATTAGCTGGACATGGT | GCAATCCAGAGGCTGAGGCAGGAGGATTATTGA |
| | GGCAGCCACCTGCTCTAGTCCCAGCTACTCG | GTTCCAGGTCAGCTGGGTCTAAACACAGGAGGA |
| | GGAGGCTGAGGCAGGAGAATGGCATGAACCC | AAGTAGAGCTTTGAGTGGACACCATGTTCAGAT |
| | GGGAGGCGGAGCTTGCAGTGAGCCGAGCCCG | GCTCAATGATCTTCAGAGTTATGCTTTTGGCAG |
| | CGCCACTGCACTCCAGCCTGGGCGATAGAGC | ACACCACACCAACAGAAAAACAAGAACAACAAT |
| | GAGACTCTGCCTCAAAAAAAAAAAAAGAATA | TGCCTTCAAAGGGAGGGCAGCCTTGTGAAGCTC |
| | TTAGTGAATGATTAGTATATGGGAAACACCT | TGATTCAAAGGAGAATTGTCCTTTGGAGTCTGA |
| | CCGGACCACCCTACATTATTATTAGTCTTCA | ATGAATTTGGACCGCTCTTTCTGAGCCTTTCCA |
| | CTTTGTGGTGGGTAAAGATAAAATAAAAGTA | ATTCTACTGGCATCCACAACTGAAAACAAACAG |
| | GCTACCGTTTATTGAATGTTTACCATGTGTG | CGGTGCCCTGATTGCCACAGACACTCTCTGCTG |
| | GATGAAAACCATGTTAATCATTGTCTTCTTT | GGCAGACAGCACACCGCAGTTCCCAGGCTGTTC |
| | AATCCTCACAGCAACCTAATGAAGTAGGTAC | TGCCAGCATCTCTCAGGTGTTCAGCCTGGGTGG |
| | TATAATTTTGCAGATAGCCACATTGAGGGTG | GGAATTGCAACATGTGTAGCAAGCCAGGTGGCC |
| | AGTGAGGTTAAACAACTTGCTCATATGACTC | CTGCAGAGCCTGTCTCCAACTTCGATGCTGCTG |
| | AAAAGTTTGGAAGCCATTTTCAAATCAGATG | GGGACACAAAGAACATTAGGGCATGGAGTGGCT |
| | TGGACAAAGTGTGCCTTTTTAACCATTGTAT | CTGTCAGTCTCTGTGAGGGAAGCCCTTGCTCAC |
| | TATTCAGTCTTCCTATGAAGACACGCCTCTA | CACATAACATCATTCCCTAGGTGTGTTCCTGCA |
| | TTTGGGGCATTTACTTCCTATATAACTTGAT | CATATCCTAATTTGTTTTAACTCTGTATTTATA |
| | GAAAAAAAACCCAGCATTTTCATTGCTTGCC | GTGAGAATTGTTAAGAGAATCTTAGGACTGAGC |
| | TATAAAAACTCTAAAGGTGTTTCTGTGGGAG | AGGACTGAACCAGACAGAGACAGCAGTTCCATG |
| | GGTGTGTTATTCCACTCAGCTATTGATAAAT | TTGCCAGACAGATCTTACACAGGCTTAGCCTGG |
| | ATAGTCCTGTCTTAATGTTTAATGTGGATCT | TCGCAGCCACCAGACCAGGTCCCTGTTCAGTGA |
| | TTTTTCTGTTTCATGCTTTTCTGAATTTTTG | GAGGTGGAAAGAAATACACATGGATTTTTTTTT |
| | AGTGACCATGTCACTCAGAAAAGCTTTGAAT | TCATTTTTTGCTTTGTAAATCATGTGGGAGATG |
| | CAGCAACATTTCCAGTGGACTGTAGGGAAAG | GAAAAGTTTACACATAGATTTTTTTTTTCTTTT |
| | CCTGTTGTTTTGGTGGAAAGTAGAGAGTCAC | CGTTATTTGTTTTATAAGTCATTACTCACTAGC |
| | AGATCCCCAACCTTCATCTGAGCCGTGGTTC | CTAGGCTAGCTTGGAGCACTCTCTGTAGCTCAG |
| | TGCATCAGTACAGACAGGAAACCAACTATTA | GCTGGCCTTGAACTCTTAGCATCTCAGCTTCAG |
| | GGAGCCACTACATGAAATAGTATTTCCTCAG | CCTCCTGAGAACTGGGATTACATAGCTATGATA |
| | GTGAGCAAAAAATTCTTTTGCTTTTGTAGAT | CTATACCTGGCGCCCAGATGTGTTTAAAAGCCT |
| | TGGCCCTGTCTATACGTGGTAGCCACTAGTC | CAACTTCCCAATAGACCTAGACGCTCCTTTCTC |
| | ACATGTGGCTTTTGACGTTTGCATTTTAATT | AGTCTGAAGGACACAAATGTACCTCAATCTACA |
| | AATTAAAGTGAAACACAATTTAAAGTTCAGT | AACTTAATCACAAATCTCTCAAGGGTGTTTCTG |
| | CACCCCTGCCACACTATAAGTGCCCAGTATT | AAACTTCAGAGCACTTTGGAACAAACTTTCCTA |
| | AATGCACAACTAGAAGTATTAGCAAGTCTGG | GTGGGGAGGTTTGTTTCTTCACTCATTTAACTG |
| | CAATACAACTGCCCAGTGGCTGCCATGCTGG | GCAAAGTCACAACTATACAACTTCATTTATTTA |
| | GCGGCGCAAACGTAGAGCACTTCTGTCCTGG | TATAATTCTATCTAACTAATGGAAATAAGAGGT |
| | CTGAAAATTCTACTAGACAGAGCCATCCAGG | GAGGTTAGAGAAGAGGAATAACTTTTAATATTC |
| | AATTTGGACTAGCAAGCACCAAGTTCACAGT | TGTAGTAAAGTAGTGAAG |
| | TAGAGAACACAGTTGCAGGCCAGGCGCGGTG | (SEQ ID NO: 17) |
| | GCTCACGCCTGTAATCCCAGCACTTTGGGAG | |
| | GCCAAGGCGGATGGATCACGAAATCAGGAGT | |
| | TTGAGACCAGCCTGGCCAGCACGGTGAAACC | |
| | CCATCTCTACTAAAAATACAAAAAATTAGCC | |
| | AGGCATGGTGGTGCTCACCTGTAATCCCAGC | |
| | TACTCGGGAGGCTGAGGCAGAAGAATCACTT | |
| | GAACCCAGGAGGCGGAGGTTGCAGTGAGCTG | |
| | AGATTGCGTCACTGCACTCCAGCCTGGGCAA | |
| | TAGAGCAAGACTCTGTCTCAAAAAAAAAAAA | |
| | AAAAAAAAAAAAAAGGAAAGAAAAAGAAAAA | |
| | AGAGAAGACAGCTGCTTTACAAAGCAAGAGG | |
| | GCTTCAAGAATCTGGAAACCAAAGGAGCAAT | |
| | GTCCTTTGAGTTTCTACAAATTTGGGCCACA | |
| | CTGATTGGGCCTTTCCACAGCCAATTCCATT | |
| | TGCCTTCATTATGGAAAGTAAACAGTTTAAC | |
| | TTCCTACTGACATGCTCTGCAGTGCAGACAG | |
| | TAAACAGTAGCTCACCGCTGCTTCTGCCAGC | |
| | TGCTCTCGGGTGTTCTACTTGGGTGGGGAAC | |
| | AGCAGCACTGGCACTGGCACTGGCCCCGGTG | |
| | GCCCCACAGAGCATGGCTCCATCAGGCTGGG | |
| | TGCTACAGAGGGATGCCAAGAACATTTGGGC | |
| | ATTGAATGCCTCTCTCTCTCTCTCTCTCTGA | |
| | AATGAAAACCCTCATCAATTCAACAATAGTT | |
| | TCTCTAATAGAACATATAGTGATTTGTTTCA | |
| | TCTCAACTGTTCCCATACAATAATAGAAAGG | |
| | AGGGAGTCTGTGCCTGAGAGTGCCTGCAAAC | |
| | CCCAGGGCACACCAGCCCCGTGGAGCCATAA | |
| | CAGTTGCTCACAGAGACAGCCCCTCACAGCA | |
| | GCCCCCGGCACAGTGACTCGTGTAATGAAAG | |
| | CTGGAAAATTGCCCAGGAAAACCTGAAGATG | |
| | CATTCCTGAAGCTCCCACACTCCAACGCACG | |
| | CACACACAGACTTCTCTCCTGGCTTTAGGAA | |
| | CATGAATTTACCTTGAATCTTTAAACTTAAT | |
| | TGAAAATCTTGCAAAATAACGAGCTTTCCTT | |
| | TGAATCTTCATGGCACTTTGTAATAAAATGT | |
| | CTAAAAGGGGGCCATTCCATGAAATCATTTA | |
| | ATTGGCATTAATAGTACACTATTACTTCATA | |
| | TAAAATCATAATCATATAAATGTACTTATAT | |
| | AACTCCATGTAAATTAATTTATATAA (SEQ | |
| | ID NO: 16) | |
| G | N/A | GACTTGCAGTCTTCAAGAACGGATGATGCCCCA |
| | | GGCAAAAGGGGTATCCTACCCTGCCACTTAGTG |
| | | GGCCCCAAAGGAGAGGCTTCTGCTCTAGGGCAA |
| | | AGCTTCATTTCCCTCTTCCTTTGAGCTCACTTA |
| | | TTTGGAATGAGTATGTCTGCCCCTTGCCTGCCC |
| | | TATCATGGTCTTTTGGGAACACACAACAAACCT |
| | | GGTTTTGCCGGTTCACAGCCAGAGGACGGATTC |
| | | CCTTCTACATGGGTCTGCCTATACCAGATGATG |
| | | TGATACTGTGTTGACTTGGGACTTGGAGTGGTT |
| | | TGGGCATGGGTTAAGACTTTGGGCCAGTTGGGA |
| | | TGGGGTAAGTGCGTTTAGCATGTGAGGATGCTA |
| | | AATATGAACTTGGGGGACATAGAGAATATGGAG |
| | | TTATAGACCCAGTGGTATCCTTCCAGATTTGTA |
| | | ATTAAATCTGTACAGTTCAATACCTCAAAATGT |
| | | GACTATATTTGGAGACAGGGCTTCCATGGGGAG |
| | | ATGACATTGAAATGGGGCCGTCAGGATGGACTC |
| | | TAACCTGAATGATGTCTTTGTAAGAGAATCATT |
| | | AGCTACAAAGAGAGCCCAGGGGCACACACTTAG |
| | | AAAGGATCCCACAAGGACACAGGAAGGGAGTGG |
| | | ACATGTGCAAGGCAGGCAGAGGCCTCCTGAGAA |
| | | ATCGGTTCTGTCTGCACCTTGATCTTGGATATC |
| | | CAGCCTCTAGAATTATGAATGCATTGCCTTCTT |
| | | TGACAAATCTGTATCTAAAAGAAAGGAGGGTGT |
| | | TATTTGTTTTAGCTCAAGTTCTAGTACAAGGTC |
| | | ACTTGGCCCCTTGTGCTTGGGTGGAGCATCATA |
| | | ACATTTGGCAGAAGACAGCCATTCGTGTCATAG |
| | | GAGATAGGATGCAGAGGACAAGTGGAAGGGGAG |
| | | GGGACTGGACACATAGGCACAACACCCGTGGTG |
| | | ACCTGCTTACCCCAGCTGGGCCGATACCTCCTG |
| | | AGATTCCAGCACCATCCAAAACAGCACCATGAG |
| | | CAGGAGAACAGATTTGAGAGCCATTATGCATGC |
| | | AAGCCATAACAGTGAGGGAATACATTTCTGCTA |
| | | AGTCATAAGTAATACTGACTTCAATCTTAAAAT |
| | | CCCAGGGAAGCTGATGAAGCTCAGCGGTAAGGC |
| | | ACTTGCTGGCGTGCTAGAGGCTCTGGGTTCCCA |
| | | TCCCTCCCAGACAATTTACCAGAGTCTTCCCTT |
| | | GGTGTTAGCAGTTTTGGGTCCTCTTGTCTTCAC |
| | | ATTAAAACTGACATTCACATGGAATGATTTTTG |
| | | CTAATGGTGAGAAAGGGTTCATTTTATTCTCAT |
| | | TAAGAGGGTCAACTAAGTACCACACACACACAC |
| | | ACACACACACACACACACACACCCCACAGATTA |
| | | TTTGCAGCCCCTCGGTCTTAAGTGATGCAATTG |
| | | CTGTGCACTCCTGTCTTGCAGGCTGTGCTCTGT |
| | | TCTATTGGTGGTTCACCAGCCTGTGCCAACACT |
| | | GACTGGAAGAACAAGCTCTCTCTGGTTCATCTT |
| | | CACAGTCTTGGTTATT (SEQ ID NO: 18) |
| H | GAATGTTTACATGTACATTTCAAACCCAGTT | GAGTATATATGTTTCTAAGCCAGGTTCCTAACT |
| | TTCTAATTGTGCAGTCTTAATTTCCTAGTTA | ATGTAGTATTAATTTCCTAATGAAACACCCTTT |
| | ATTTCACTTTACAGATAAGAAGCTCTGGAGA | ACAGGTAGTGAGGCCTTTGGAGACCAGGGCTTT |
| | CATGGCCTTTCCGGTTAAAGACACAGAGCCC | AAAGGCCAAGTAGCTGAAGCCCAGGGTCTTTCC |
| | AGGCACTGCCCACGGCTTCCTCCACACTCAT | ATGGCTTCTTCCTATGACTGTTTATCTAATAGA |
| | GCTGCTTTCCCTTAGGTAAGACAAACCTCAC | TGAGACAAACCTTTTCAAAACTGATTATCAGTT |
| | CAAAGCTGAGACTGGCTCAAGAAACGGGGAA | AAGTTCCAAGAAAGCACCACTGTAAATGTTAAT |
| | GCCTAATGCTTGTAAACATTCCCTTAATTGG | GTTCCTTTGAAATGGAAGTATTTAGCGCTCTGT |
| | AAGCATTAGGCACCAAAATTCTTCCTAAAAA | GTGTGTGTGTGAGTGTGTGTGTGTTGTGCAGTT |
| | ATATGTAAGCCCCAAGAATGAAAGGGCCATG | GGGTACATATATGCAGATATGCACAATTGTTTG |
| | GTTAGCACAAACCGCACCTCCTGAGCCCAGC | TGTTTGTGGGTCTTTGTGTGTGTGTGCAGGTCT |
| | AAAACCCAACAGGCACAGTGCAGCACAGCCT | AAAGTTTTTCTTTTCATTAGTTATGGTCTAAAG |
| | GGGCGGTCTCTCAGGTGAGTCTCTGCCTCGC | TGGTTTTAAAAAAAGAAAAAGAAGAGCAGAGAA |
| | TCTTGCCCTGTCTGTCACCTCATCTCTGCCA | GGCTATGATAGCATGAGGTTCCTTTGGGATTGT |
| | AGTCTGAAAATCCTGAGCTCCAGGGACTGTG | CTGGCTTAGAACGCTAGGTTTTCCCATGTTTTA |
| | GGAACTTCACTAGACATGTGTGAACAACTCT | ACAGCTTCCCATGTCCTTCCCACTCTGCCTTTG |
| | ACATTCTGATCCGTAGCGTCTCCCTAATGAT | TCTTTCTCATTGTGATCCAGATTTGCCCCAGAG |
| | GCACATCTAGGAAGGAGAGGGAGGGAGAGGG | GGGGAGAACCCAGTAGGTAAGAGTTCACGCTGT |
| | AGCGTGTGCATTCCTTGGAGCAACGAGGACA | ACTTCCATGTTAATTAAGTGATGTGGAAGTCTT |
| | GCCTAGTGATTTGCAAACTCTTTGCGGCCTC | GGAAAGGCTGGGCAGTTTTTCCTGTCTTCCCAG |
| | CTGGTGGGCTTCAGAATCAATTTGTGAGTCC | GAGCTGGGGGAGGTTCATCCTTAATGGAACCAG |
| | CAACCAGAATTTTCTACATAATTAGAATAAA | TTCCATGCCATCCCCAGGAGGCAAGAAGTCTGG |
| | ACAGAGTTAAGATATGAGTGCATCGTATGTT | AAACATCAATAATTATTCAGTCACAACAACCCA |
| | GCAAGATACTGTTTTGTAAACGTTGTTTCAG | CTTTCCTCTCTCCCCCTAATCCTCAACTGCTGA |
| | ATATTTGTGAGTGCACATGTGTGTGTGCAGT | CTTCAGGACAAAGTCCATCTGATTTCAATCAGA |
| | AATGGGTCACAAAATATATTTACTCTGGGTC | TAGGAAGACTAGTTAGAGGCCTGCCCCAGTTTA |
| | ATGTTTTAAGAGGGCTAGAAGGCAACACTAA | CTGGCTGCAGCAACAGGAAGCACAGGTTACAAT |
| | CATAGGATGGTTGGAAGATGGTCAGGCTCAG | ACCAAGTGATTCCACGCTGAAAGCTTCACTCTG |
| | AACATCAGATTTTGCCTCCTTCCAGGGTACC | ATCATCCTACCAGGCTGCTACATGAGCCCTTGA |
| | ACTTTTATCAAGTCACACATTCCTTCCCGCT | AAGCGAATTATCCCCGGAGACTTACTTTCTATA |
| | CTGCTTTTGTGTTTCTCAATCGCTATCCAAA | TAACACATATATACTTACATATACATGTCGACT |
| | TTTGCGCAGAAGTCAGGAATCACGTGGGTAA | TTGTTTTTTCTTGTATGCTGTAAAGATGCCTAG |
| | AGATTTAAGCTGTACTTCTGTGTTAATTAAG | GATACATTTAAGGATGCAACATAAAAGTCACTT |
| | CACGTTGAAGAAGAGGTGCTCTGGGGGAACG | TCTTCATGGAGTAATTATTATAATAGTACTTGT |
| | TGGAGAAGGTGGGTAGCGAGGGCTCCAGGGG | TTCTGGGGGAGCAAATTGAAATGTTTCCCAGTG |
| | CTCAGAAGGTGGCCTCGAGGGGCTCTCATCT | TGAACTGCCAAGTTAAAACAACAAAAAGCTAGT |
| | GCCATCCTTGTGAGGGAGAAAGTCCTAAACC | TGGAGCTCCCCCT (SEQ ID NO: 20) |
| | AGTCGTAACATTGCCAGAACAAGGGGTCCCA | |
| | ATCCAGACCTCCAAAGAGGGTGCTTGGATCT | |
| | CTCATGGGAAGGAATTCAAGGTGAGTCACAA | |
| | AGTGCTGTGAGAAGAGAGAGTTTTTTGGAAG | |
| | TTACGCAGATACAGAGTAGGGTGTCCTCAGA | |
| | AAGCAAGAGGAGGAACTGCCTCGTCTTTAAG | |
| | TTTTTCTTACATAGGAGTCCTCTCTATGTAA | |
| | AGACAGAGCTAAGCTGTGTCTCTATGTGGGT | |
| | GGGCTGACAGCGTGACAAAATTTATTATTCT | |
| | GTTGATTTAAAGAAAACTATACTCAATATTT | |
| | TAATGTGTAAGTACATCAAGTCATAATTATA | |
| | ATTATCTTGAAAGCATATATTGTTATGGGTA | |
| | TTGGGACCTCTGGACTTTTCGTTGTCATATG | |
| | ATTGTATCCTTGCAGGTATCTTTAGGCTGTT | |
| | TCTTCAACTGTAAATATCTTATGACTGTGGG | |
| | TCGTGACCGGCAAGGAATGGAGTTGGTTTTT | |
| | AAAATGGTGTCACCCTGGCTCTTCTATGCTC | |
| | CTGTTTCCCTAACAGTAATAGCCCAGCCATT | |
| | CTCTCCCATGTTCTCCTCTGCCCTCAACTTC | |
| | AGAATGAAGTCAATTTTTATTTCAGCCAAAA | |
| | TAGGAGGATTCTATTCTGTCTGTTGAGGTCT | |
| | GCTGTGGTCTAATGATGTTAATAACCAGTGG | |
| | CTGGGCATGATTACACGACGAGGATTCTAAA | |
| | TCCTGTTTCATGTTTCCCTCTGGGCCCACTG | |
| | GCTATATGACCCCTTAAA (SEQ ID NO: 19) | |
| I | CTAACATAGGGTCGTTAGTGTCAGAACTGAA | TCCTTGGCTACTTTCTCTAGCTCCTCCATTGGG |
| | TTAAATTGTAGGACATGCAGGTGGTGACTGC | AGCCCTATGATCCATCCATTAGCTGACTGATGA |
| | AGAGAATTGGAGCATTGCTTGGAGTGAAAAC | CACTGCATTCTTTAATATATGGGGTTTGCACTA |
| | CAAGCCCACATATTTGGTGTCAAAAGTGTTA | ACTTGGGGTAGTTATTGTCATGTTTGAACTAAA |
| | TACAAGTAGAAAAACAGGTTCTCTTTAATGG | TTATAGGACCTCCAGTTGCTGGAGAATTGCTCT |
| | AATATTATTCAGCCGTATTAAGGAATGAGGT | GTGTGGACTGTCCACACATATTTGGTTTCTAAA |
| | TCAGACCCATACTACAGCACATATGAATCTC | ATGTCATATAAGCAGACACTGCAGTTTCTCCAC |
| | CAAAATATTGTGTTTAGTGAAATAATATAGA | AGTGGAATCTTACCCGGGCATAATAAGGGAAGA |
| | CACAAAGGACAAATACTGTATAATTGCACTT | CATTCGGCACAAGCTTCAACACAGGTGAACCTT |
| | ACATGAGGTGCCTGGAATAGGCAAATCCATA | AGAAAACATGCTAGTGAAATAATCCACACCCCA |
| | GAGACAGGCAGTAGAATCATGGTTGCCAGGG | AAGGACAAACAGGAAATGATTCTTATACAAGAC |
| | GCTGGGCGGGAGGGAGAATGGAGAGTTAGTG | ACCTGGCAGAGGCCAGCTTAAAGAGACAGGCAG |
| | CTTAATGGGTACAGAGTTTCTGTTTAGAGGT | AAGATGTGAGTCCCAAGGACTGCGGAGGGGAAA |
| | GATGAAAACAGTTTGGAAATAGTGGTGATGA | TGACAGCCAGTGTTTTGTGGGTGCTGAGGGCAA |
| | TTGTACTATATTGTGAATGTATGTAATGCCA | CAGTTTGGAGTAGACAATGGTGATGCAGGGCTG |
| | CTCACCGAACACTCTAAAGTGTTTGAAATAG | TGAACGGGCTCAGTGCCGCTCACTGAACCAAAC |
| | CAAATTTCTATTATACGTATTTTACCATAGT | AGCCTAAGTGTTTATAATAACAAAAGTAATACT |
| | TTTTAAGTTAATTACCATAGTTTTTAAAAGT | GACATACACCTTCCGTTGTTTGAAAGAGTTAAT |
| | TAATAGGATAATATTCCCTGAACCACTATAC | AAGGTAACATTCCCCAAATCACTTTAAACAGGC |
| | ACTTTAGATTGGTACACTGTGTGGCATGTGC | AAACTATGTGAAATATAAATCTGTTTCTGTGAA |
| | ATTATATCTCAATGAAGTTGTTAAAAACAAG | GCTGCTTTTTTAAATGCTTCTCCTATCAGAGGT |
| | ATTTAAAAGCAGAGATTGGGTAAAGTAAAGG | CAGAAGAAAGAAGGCTTGCTGGGAGTGGAGTTG |
| | TTTGCTCTGTGCTGAGCTGTGTGGCATGTGG | GCTGTGTATCTCAGACCTGTTTTTGCAGGAGGA |
| | ACCTGTTTTCCCAGGAGGGAGCACTCCTGGG | GTGTGCGCTCCGGGATTTGGCAGCGGCTCGAGT |
| | GTTTTGGCCGCAGCTGCACATCAGCCCCCTG | CATCCCTGTGAGAGGCAGGCATGGTGCGTGATC |
| | TGCAGAGGAGGTATGGTGTGTGATCTGGAGA | CTGGGGCTTTTCTGTTTCTAGTGTTCTATTTAT |
| | TTAGCTGTTTCTAGTGCAGTATTTACATTTA | TTTAAAGACATTGCTGAGTTCAGCAGAAATGTT |
| | AAGACATTGCTGAGTTAGGCAGAATTTTCTA | TCACATCCATTTGTATTTTCCTTGGTACTCATT |
| | TATCCATTTGTATTTTGCTTGGCATTCACTT | TCCTTACAAAAATGACGATCAAAGCAAAGAAAA |
| | TCTTACAAAAATGGACAATCAAGACAAAGAA | CAGAGAATCTTCATTTTACCCCAAAGCAAAGTG |
| | AACAAAAGGTCCAATTACTACTCTTCATTTC | AGTGCACTTCTAATACCATAACAGAAAAAACGC |
| | ACCCCAAAGCAAAACAATATTAGTTTTCAAT | TTCGGGCCCTTAGGAAGTGCTGAAGAAGCTGGG |
| | TTTTTTTTCCCATAGAAAGCAATAACAGTCC | CAAGGTGGTGGGTGCCTTTAGACCCAAAGGAAA |
| | CATACTACCTCCTCTTCCATGAAAGTAGTGC | GTGATTTTCTCCAAATGTGAGAGGCCTGCGATG |
| | TTGAGATGCCCCAAGGAAAAACCATTCTTTC | ATGGGGTGAGTGGCCCCCAGAGGATGTGGGGAC |
| | CAAAGATGAAAGACTTTGTACCTGTCAGGTG | TGACTAGCGCTGTCTCCGTCTGTATGCCCAGTG |
| | AAGAGATGGAATAAATGCCACTCCTAGTGGG | AAGCTGTGGGTGGGACACAATTAACAGCACAAG |
| | TGTGGGACTTGTGCAGCCCCTGGTCCCCAGT | TCTGAGTGGTGAGACCCTCTGCTGTGACGAACC |
| | TATCTGCTTATCAGAATGTGGTTTGCATATC | CTGCACTGATGTTACTGTTGAAGGTATCTCTCA |
| | ACCTTTAGCGGAATTCCTTGGGATGCTTGTA | AGTGCTCATGCTGGAAACTAAGCCCCCAGTTTC |
| | ATTCTGGGGGAGATGTCTGGAGTCTGCATTT | TAGTTGATGTTGTTTGGAGGTGGGATCTTATGG |
| | TTAGCCAGTACTCCTATGACTTAGGCACAGT | GAGGGGATTAGGATTAGATGATGTCATAGGGGT |
| | AGGGAACCACTGGTGCCATTCCTTCCTTCCT | GGGGCCTCCACAATGGCATTAATTGCTTTAGAG |
| | TTCTTCCTTCCTTCCTTCCTTCCTTCTTTCC | GAAGCAGACAAGACCAAACTAGCACATTTACGC |
| | TTCCTTCCTTCCTTCCTCCCTCCCTCCGTCC | TGTCTTACCGTGAGAGTAATCTGCCATCTTCTG |
| | TTCCCTCCCTCCTTCTTTCTCTCTTTCTTTC | AGGCAGGTGAGTTGATATCACCAGATGCCCACA |
| | TTTCTTCGGAGTCTCACTCTGTCACCCAAGC | CCATGCATTTGGGCTCCACAGTCTCCAGAATCA |
| | TGGATTGCAATGGTGTGATCTTGGCTCACTG | TAGGTTTTGAACCTTTATTCTTTATAAGTTTTC |
| | CAACCTCTGTCTTCTGGGTTCAAGTGATTCT | TAGACTGGGGCATTCTGTTACAGCAGCAAGAAC |
| | CCTGCCTCAGCCTGCTCAGTAGCTGGTATTA | TAGACTAATATACATCCCTCCTTCCATCTGCCC |
| | TAGGTGTGCACCACCACACCCAGCTAATTTT | A (SEQ ID NO: 22) |
| | TTTGGATTTTAGTGGAGGGGTTTCACCACGT | |
| | TGAGCAGGCTGATCTTGAACTCCTGGCTTCA | |
| | AATGATCCACCCGCCTCAGCCTCCCAAAGTA | |
| | CTTGGATTACAGGCGTGAACCACTGCGCCCT | |
| | GCTGCAATGCTTTTGCTTTCCGTATACAAGG | |
| | AGGGGTTGCAGGCTTGACTCTAAAATGATTG | |
| | ACTTTATGGAGGACCGTCTCATGTCTGGATG | |
| | GTAAGTGATAGGGGAGGGGGCAACCCTAAAT | |
| | GGGATCCCAATGACTTGATGAAAGACTGGAA | |
| | GATGAGACACTTTCAGGTGTGCATAATGGAA | |
| | GACTTACGTAGGACTAGGACCAAGCCTCTCA | |
| | ATTATACTAAGTTGTCCATGATTGACCAGGG | |
| | ATTTGATGAAAATCCCACTGCCTTCCTAGAA | |
| | AGGTTAAGAGAGGCCTTGGTAAAGCACACCT | |
| | CTCTATCTCCTGATTCAGTCAAGGGACAGCT | |
| | AATCCTAAAGGATGAATTTGGCTGGGCATGG | |
| | TGGCTCATGCGTGTAATCCCAGCACTTTGGG | |
| | AGGCTGAGGTGGGAGGATCACCTGAGGTCAA | |
| | GAGTTTGAGACCAGCCTTGTCAACGTGGTGA | |
| | AACCCTGTCTCTACTAAAAATACAAAAAAAA | |
| | TTAGCTGGGTGTGGTGGCAGGTGCCTGTAAT | |
| | CTCAGCTACTCGGGAGGTGGAGGCAGGAGAA | |
| | TTGTTTGAATCTGGGAGGCAGAGGTTTGCAG | |
| | GGAACCTAGATCGCACCATTGCACTCCAACC | |
| | TGGGTGACAAGCAAAACTCCATCTCAAAAAA | |
| | ATAAAAGGGATAAATTTATTACTCAAGCTGC | |
| | CCGATATCAGGAGGAAGTTGCAGAAAGGGGC | |
| | CCTGGGTCCAGAAAGTACATTAGAGGACCTC | |
| | CTGAAAATGGCCACCTTGGTCTTTTATGATT | |
| | GAGACAGGGAGGCCTGGGAAAGAGAGAGGAG | |
| | ATACAGGTATTCCAGGGTGCACCTGTTAACT | |
| | TCTAAAGATATGGCAAGAACAGTTCTCTCTC | |
| | TTCTAAAGTTTATCTGCCCCCGTACAAGGTT | |
| | TAATTTCTTTCACCAGGGTGAAACAGCTTGG | |
| | AGTACAATGTTGTTGTTAGTATATTTCACTT | |
| | ATCTCTGTTGGCACTAAATTCTTTCCTTGTA | |
| | TAATACACATGTTTAACTTATGCATACTTGA | |
| | CCTTATAAAACTTGTTTTTTTCTCTCATGCC | |
| | TAGAAGCCATCAAACTCCAAATGGTCAGGCA | |
| | ACTGGAGCCTCAGATGATAGCTCCCCTTTGC | |
| | TAGGAACCCTTAAATAGACCTCTGGGAGGAC | |
| | TCTGACTGCCATTTTCTCCAAAACAACACCC | |
| | CTTGTCAGCAGGAAGCAGCAAGACTGGTCAT | |
| | CAACCATATTCTAACGGCAGTATTCCTATGA | |
| | TTTAGCCAGTGGGCCGTGACCGGCAAGGAAT | |
| | GTGCCTTGTTAGTTTCAAGATGGAGTTGATT | |
| | TTTAAAATCATGTCACCCTGGCTCTTCTATG | |
| | CTCCTGTTCCCCTAACAGTAATAGCCCAGCC | |
| | ATTCTCTGCCATGTTTTCCTCTGCCCCCAGC | |
| | TTCCGAATGAAGTCAATTTTTATTTCTTCAA | |
| | CGTACCTCTTCAGAGGGGAAATTATACAGGA | |
| | GGGGGGCAGGGAAGTGCTGGGTAGAGAAAGG | |
| | TGGATCCCCAGCTAGGGTTCCACCCCCACAG | |
| | ACCTAGGTGAGGAAAGGCACTTCTGGCTTCA | |
| | CACCCAAATGTTGCATTTTCGAAGACCAACC | |
| | TGGCCTGCCATGCCCCCATTCTGGGCCTATA | |
| | AAAACCCACCACCCTAGCGGACAGACACACA | |
| | GGTGGCCAGACGTCAAGAACAGCACATCAGC | |
| | AGTTGAAGACACAAAAGGGTGGACGACAAGA | |
| | AGGCATCACAAGAGAACGTCAAGGGAGCACG | |
| | CCGATGGAAGAACCTGCTGGCAGGCTATCCA | |
| | CTGTTGGCATGAGGGGGAGTTTGGCTGGGGC | |
| | AGTCAGAGAAGAGCCCGGCTGCATAGCGGCC | |
| | CAATTCCAGGGGAAAACCATCTCTCTTTTGG | |
| | CTCCCCCGGCAGAGAGCTACTTCTGCTCAAT | |
| | AAAACTTGGCTTTTATTCACCAAGCCCAGGT | |
| | GTGATCCGATTCTTCCGGTACACCAAAGCAA | |
| | GAATCCCTCTGTCCTTGTGACAAGGTAGAGG | |
| | GTCTAATTGAGCTGGTTAATACAAGCCACCT | |
| | ATAGAGAGCAAACTAAGAAAGCACCCTGTAA | |
| | CACAGGCCCACTGGGGCTTCAGGAGCTGTAA | |
| | ACATTCACCCCTAGACACTGCCGTGGGGTCG | |
| | GAGCCCCCCAGCCTGCCTATCTGTATGCTCC | |
| | CCTAGAGGTTTGTGCAGTGAGGCACTGAGGA | |
| | AGTGAGCCATACTCCCATCCACGCCCTACAA | |
| | AGGGGATAAGGGAATCTTTCCTGTTTCATAA | |
| | GTAGCAATCTCTGTGGTAACAGCCCCTGTGG | |
| | TGATGCCGTCTCTCTCGGTTCTGCCCT | |
| | (SEQ ID NO: 21) | |
| J | N/A | TGTGTGCACCAGCTTTGACTGCTGCTGGAGGCT |
| | | GCCCATTTCCTGTGATCTCAACCAGCTTTTCTG |
| | | ATAGGCCAGTTTATCTCTGGACTCTGGCCTATG |
| | | CCTGATACAGATGTAATCAGGCATCCAGGAAGC |
| | | TATCTATATGGAGGCAAAGGTCCTTTTATTCAG |
| | | GCCACTGGAAGCCTCTTCCATAAAGTTCAGTAG |
| | | TACGAGTACAGTGTCCTTTCCTGTGTACAGCCC |
| | | CTCGCTTTCTCTTCTGGACTCCCAGCTGAGCCA |
| | | GTGTTTGAGCCACCCATCACTCTGAAAACAGCA |
| | | TCTTCATCTCCTTAGGCTCAGCTTCTCAAGTCA |
| | | CACAGGCTACATTGCTGCCCTCAGGGTGAGCCT |
| | | CCCTTCATTCATCTCGGTGATAATTCTAAACAA |
| | | TGGCCTGTGTGTTATAGAAAGGCCCTGCAAGCA |
| | | TACATGTTATCAACTTACTAGCTGTGCCCAAGG |
| | | TTGCATAGCTAGTAAGTGGTAAGACTGAAATTT |
| | | GAGCCTAGGGGACCATAACTCTAAACAATGTTC |
| | | TATCCACTAGGCGGTACTGTGTAGACCATGGGC |
| | | TCACACACACACACACACACACACACACAAAAT |
| | | GTATTGAATAAAATAATTGTGGGTTTTGCATAT |
| | | TTTCCTGTTTTATGTCAGCTTGACACAAGCTAG |
| | | AATCATTTGTGAAGAGGGACTCTCAATTGAGAA |
| | | AATGCTTCCACTTTTTGTTGTTTTGTTTGTTGT |
| | | TTTTGCCTGTCGGAAAGTCTGCACT (SEQ TD |
| | | NO: 23) |
| K | CTGTGGAGTGCCTATAGCACTGTGTGTAGGC | TCCCAGAGAACCTAAGCCTGATTCCCAGCACCC |
| | AGAATGCAAAGGGGACAGTGTGGGTGGGGAC | AAAGGACTGCTTACAACCAACTGAAACTCCAGT |
| | AGTGTTGGTGTAGAAATGGCGGGGAGGTTAG | TCAGGGATCCAACACCCTCTTCTGGCCTCTGTA |
| | ATTGCAGGCACAGAGGGCCTCAGCCATCTCG | GGCACCAGGCTTGCATGTGGTACCCAGACATTC |
| | AGAGCCCAGACTTCCTCCCTGAGGTGATGGC | GTGCAAGCAAAACACTCATACATATAAAAATAG |
| | ACTTGGGGAAGTCAGTCATGGAAGGATTTTA | ATAAATAAATGCCTATTTAAAACCCTTGCCTCA |
| | AGAAAGATGTGAAAGGGGCAGGTTTCTATTT | TCTGAAATTATCTGAATGTTGATTTCTTTGGAT |
| | TCAGAAAACCATTCTGGGCCAGTGGAAGATG | TCCCTTTCCTTTTGCCCTTGGGAAAAATAGGTC |
| | GAGTACACAGGACCACACCTTGGTGAAGGGA | ACCCCTGTGTCAGTTACTGTATGTTTTGGTCAC |
| | GATTGTAGGAGCCTGGGCTTGGTGGCGGGGG | TGTTCATAGTTTTAGAGAGGATGTCTAGGAGGG |
| | ACAGTGGAGAGAACAGCCTGGGATGTATGAA | CAGGGTCACCTGTGGTGTGGCAATTGGGAGCTC |
| | CATGGCAAGTCTCCCTTCCTGGACAGTGGGG | CATGTGCAGAAGGAATGCAGACACAGCAGCAGA |
| | TTTGCCTATGGTGGACAGAAGGTGAGATCAT | GAGTGCAGGAGGCCCGGAAGGTTCCACCATCCC |
| | CCTTTGAAAAATGCCACTTCATAGTGTTTCC | CACAGCCCCACTTCCTCCCTCTGCCGAAGGGGT |
| | CCAGCTGTGGGCCTTCACTCATTGGAGGGTC | TGGGGGTCAGGCAGAGGCTTTAAGAGGGGCGTG |
| | AAATAATCAATGTATTAGGTTGCAA (SEQ | GACAGGGTAGATTTCTGTTTTGGGAAAACCATC |
| | ID NO: 24) | TATCAGAGGGCAGAGGACAGGGTGGAACCCAAC |
| | | ACAGCTGAGAGCTTGCAAGGGGCTGGGCTGGGC |
| | | AGCAGTGAAGAGGAACCTCACAGGGAGGAGCCC |
| | | CTGGGGTGCAGGGGCTCTGAAACTGCCCTGTGA |
| | | AAAACACTGCCTCATTGTCTTGGCAGTTTGGGC |
| | | CCTGACCCAGTAGCAGCAGGTCAGACAATTGTT |
| | | ATATAAAGTTCCGAAAATTCAAACCTCCCCCTT |
| | | CCTCCTTCATCCTTCTTAGCTACACGTGTGTCC |
| | | ATGAGTGGCAGAGCAGGCACTCACATAGAGGTG |
| | | TGCCCACTGCAGCGGCTACAGCACTAAAGAAAA |
| | | TCCCTCTCTCCCCTTCCTCTCCCCCTTTCTTTT |
| | | ACTTCAAAGCAGAGTCTTACTATAGGGCCCGGC |
| | | CCCTGTGGGCTGCTCACTTTTAATCCTCTGCCT |
| | | TGGCCTATCTAGCACTGAGATCACACACCTGCC |
| | | TGTGTCACTATGCCTGGCTTCCAGCACTTCTTT |
| | | GAGTGCTGACAGACACCTCAAGTGGAAAATTCT |
| | | TGTCCTTGCTTCATTTGACAGATCACAGTGAAA |
| | | ATGGGAGCCCACTAAAAATACTTTATAGGATTA |
| | | CCCTCGGGCTGTGTCTGAGGCGGGTAGGTAACA |
| | | TAAGGAATTTCAGGGTTAGACTTTAGTCCTGTC |
| | | ACCAAGACATCTATCTCTTTATACATATAAAAG |
| | | TATTCCACAGTCTGAAAAAAGCTCTGAAATAGA |
| | | GAATGCTTCTTGTCCATAGCATCATAGATAGAG |
| | | ACCCTTCAGACTTGTATATAAAACAGAATTGAA |
| | | AAGTCAATTCAGGTGTGCACACACACATGCATG |
| | | CACGCACCAGCACGCCTGACATCTCTCAGGGCT |
| | | GCCGGGCATCACTCAGGTGACTGCTTGACGTGT |
| | | TGATGTTTGTGTCTTTGGCTTCTTCTTTGAGTC |
| | | TTTTGTTTTTCTTCTTTTATTTTATTTATGAGA |
| | | CAGGGTTGAGTTCATTGCAT |
| | | (SEQ ID NO: 25) |
| L | CACACCATTGCATGCTTCAGCCGTTGCCCGT | TAAGCCATCACATGCTTCAACCATGGGCTACTT |
| | GCTATTTCCTCCCTTGGAAAGCCCTCTACTG | CCACCTGCTCCCCCCCCCCCCACACACACACAC |
| | TGAGGCCCTCACCTCTCAACCCTCTCCCTGG | TGCTACCCCTCACCCCCAGCTTGGTGCCTCACT |
| | CCCCCATGTTGTCTATGTGATTTCTTGCCAT | TCTCAGGCTATAATGCTGCTTTCATGGACATTC |
| | TTAAAAATCTACCCAGGTGTCAGCGCTTGGG | CTTGTTCTTTGGAAACAAGGGCCCTTCCCTCTG |
| | CAGTTTCCTCACACCTCTCACCCAGTTCATC | CAGAGTTCTCCTGCCTGAGGCTGTGTGTTCTTG |
| | CTCCCTTGCTTGGTGCTATTTCTGCCCTTGT | GTTTGTGGGCCTTTGCCCAGCTGGTGCCCAGTG |
| | CCATATCCCCACCACAGCATGCACTTTGGAT | CAAGGTGCCCTGCTAACTGAACAAATGACCTTG |
| | TCCAGGCACGCTCCTTGAGTGTGACCCCGAG | CTCATCGTCATCTTCTTGGTCTCCATCTTTGTG |
| | GCCCTCTGTGGGCTCTTGGAGCAGGGCAAAG | GTGGAGCCTTCTGGACCACCGGCAGGTACCCTT |
| | CTGGGTGTGCTGGGGCGCAGCACGGGCCTGA | TGCAGGACAGCCTATCCTGCCCTGTCTCCCTAC |
| | TGCCCTGAGGTTGTTTGTTGTGCTGGGCTGG | AGAGCCACTCCCTGAAGCTGCAGAAAACAAGAG |
| | AGGCGTTCGAAGAAACGTCCAAGGAGGCTGC | AGCATAGAGGTGACCCTCTCCACAGGTGTGTGG |
| | TAGACTCAGTTCTTTCTTTCTGTTTTCCCTC | CCAGAGCCACTCATCCACAGTGGCCAGGCCCAT |
| | CACCTCCTCTGCTAGTGGAAGCTCCATGTCT | CCAAATATTAATGATGGGTGTTTTCTGCTTTGA |
| | CCCAGGCTCGTGAGCTGGCAAACACCCCGCT | AGTTGAGAATGTCGGTCCTCAAGAGTCCACCCT |
| | TGCATGGTTCAGTGTTGTCGTTGGCGGCAGG | GAAGAGAACACAACCACATCTGTTTCCTTCCAG |
| | CGTACGTGGAAGGCCAGTTACAGAGGGTCTC | GGAACAGGGGCTGCACTGCCCTTCTTCTCTGTC |
| | TAGGGCTAATGCATTTCACAACACACCGCCC | CGTGCCCAGAGCATGTATCTGAGCATGCCCAGA |
| | TCTGACACTCCACGCTCTGCTTTTCCTCCAG | GCCAAACACAGCATCTATTTCCTACTGATCTTC |
| | AACCACTCCCTTTGCAAAACTCTGTTTCAAA | ACAGCTGGACAGGCTCCCACACAGCCAGATGCT |
| | CAAAAAGAGCACAAAGAGGCTGACCGTGCCT | CCCTGGGGAGCCTCAAAAGCAAGGTTCACCAGG |
| | TCCTCCAACCAAGCTCCCCTCTCCACAGGTG | TGGAGCTCTGGGGAAATTGCTTTCAACTCTGTC |
| | CACAGCAAGAGCCCTTTGTCTGTGATGGGAC | TTGGCAGGGCTTGCCTTCTGCACCTGGCTTTAG |
| | AGGCCTGGGCTCCAGTGAGCAAGACAGGCAC | GAGGGCTCCAAGATGCAGCATAACATGGGACGG |
| | TGTGGGCCCATCCAAATATTAACTGTGGACA | ATATCAACGCTTCTGTCTGATCTTATAACAAAG |
| | CTTTCCTACTTTGAAAACATGAGACTTTGTA | GTCAATTTGTAAAGTTGATACCACCAAGTCCTT |
| | CTCAGAGCCCTGCCCTCCAGAGAACACAATT | TCTTCCTTCCTTTCTTCCACACCCCGTCCTCTC |
| | ACTTCTGTTTTTCTTTTCCTAGTGGAAGGAG | TGAGAAAATGGATCCAATAGAAGCTAGAGTGTG |
| | GCTTGACACTGGTGATGGCCTTGCCTTTACA | ACTTGTAGGTTCTGACTGTCACTTCTTTGGGGT |
| | ATGCTCAGGGTTTGGGAAAGTCAGGGCCTAG | GAATTTTAATGCCAAATCAGCCAGGGGCGAAGC |
| | GGCTGCTGATCTCCAGGCACTGTCTGCTTTC | TGAGGAGAGCCAAGTTCACACACAGTTCAGCAC |
| | CATCTATCCTCTCTGCTTGGTCCCTGAAAAG | GAAGTTTTAATTCAGTCCCATCCGTCCGAATCT |
| | CAGGAGGGAGACAGGAGGAATGGGAGCATGA | GCACTGCTGTGGGTGGGTTAAAGGGAGAGCAGG |
| | ATGCCCTCAGGGTCCACGGGGGATCCCGGAA | CTCCTGACAGCATGTGCTCCAGCACAGGTGAGT |
| | GGCCTAGAACACCAGGGGTCTGGGCTCCACC | CTGTCACACTTTTTCCTACAGCTGCCAGGCAAG |
| | CATGATGGATCATGCCTTTGGGGGAAGATTG | ACGTCAAGTCTACTTAAGGTTTCTTATGCCTGG |
| | GCCTACACTCATGTCAAGTAATAAGTTTTAC | AATCGCCTAAAACGTAAAGCAATCAAAATGTCT |
| | TTCCTGCACCTGGTGTTAGGTTGGTTCTAAG | ATCACCCAAAGAGTAGCCAGACAAAACACAGCA |
| | ATGCAGCTGTAACCTGTGACTAAGATCAATA | GGTCCTTTTATGAAGAGTCCTGTGTCACAAGAC |
| | TTTTTCATGTCACTATCTGATCATACAATGG | ACAGGAATATCAATTCTCAGCCATTAAAAGGCA |
| | TCAATTTATCGATTTAGAAAATTGTTGCACA | CGCTGTAATGACACTGGCCACGATATGCCACAT |
| | ACGAGGCAACACCGAGTCATGACTTAAAAAA | CTTAGAAATATTACAATAAGTCAAAGAAGCCAG |
| | AAAAAAAGTGGATCTAACCGAAGCTAGATTG | CAGCAAAAGGCTAACTAATGTATTATTTCCAT |
| | TGGCTTATCACCTTTGATTGTCAGTTTCTTG | (SEQ ID NO: 27) |
| | GGTCAAATCTTAATGCCACATTGACCACTGT | |
| | GTCAAGAGAGGCCAGGTTCCAACTCAGCTCC | |
| | GTGTATAGTGTTCATGGAATCTCAATGCTCA | |
| | TCAGGCGCTGCTGGGGCTGGGCCTCGGGGAG | |
| | GGGCAGGCTCCTGTCAGCACAAGTCACCAGC | |
| | ACAGGTTTTAACCAGCCAGTCTGGGCTACTT | |
| | TTACCACTGAAGCAGTGGGGCGAGAAACTCT | |
| | ATTTTACAGTGTTTCTAAAACCTCTGTGAGC | |
| | TAAAAGTAGAAGCAACTCAAATGCCCCTCAC | |
| | CTGATGAATAAACAAACACAGTGTGGCATCC | |
| | TCGTACAATGGAGTATTATTCAGCCATAGAA | |
| | AGGGAGGAAATAGTTGTGCTCGATACAGTAT | |
| | GGATGAGGCTTGGAGACATGATGATAAGTGA | |
| | AAAGAAGCCAATCACAAAAGGACAAATAATG | |
| | TATGATTCCAT (SEQ ID NO: 26) | |
| M | CTCTAGGTGGTGAAAATGACCAGATTTGGTT | CCTCAGCTGGAATTAACCCTACACAGTTCCTCA |
| | GTGGGGTCATAGTGGACACTAAAGATCAGCA | GAGCCTAGGGCTTAGTAAAAAGGCCAAGCCTGA |
| | AGGGAAAAAAGATGTGACTATAAACTTTCCA | CCTATGACCTCTCTGACATCTGTCCTTAGCACG |
| | TTCTCACAGTTGTTTTGAGACCCGAGTGTAC | TGTTCTTTTCTTTCCAAGTACATTGTACCACCA |
| | GTTTAATGTTTTCAACAGAAGAGGCTGCATG | TGATGGCCTGTGCCCTCCTCCCCATCACCTCCA |
| | AAGAAGAGTAAGTTAACCGCGGGGAGGCTGT | TACAACGAATGAGCTCTCATGAGAGCAGAGTGG |
| | GAGAATTTTTCTGCGCGGACAATGGAGCTCA | AGGCTGGTGCTGTGGCCTCCACTCAGGAATTGT |
| | GTGTCTGTTTCAGTGTTTGTGCTCTCTATAG | GAACCACTCCAACCTTCTTTTGTTAAACATTAC |
| | ATACCTGGATGATTCTTGGGCCTCAGTGTGT | CTAGCCTCAAATATCTTGTGATAGCAACAGAAG |
| | TCTCGCTCCCTCCCTGCCGAGACTCAAAGGG | AGACTAAGATACTTAAAAATATCTATGGATGAA |
| | ATGATGCACGCTGCCCAGCCAAAACCAGGAC | GAAAATGACCAATGTGAGGACGTCGTGGATATT |
| | AGAACGTCTTTTTCCCCGTGGGAATGCGCTC | GGCCATCAGCAAAGAAGAGAGCATAAAGTTCCC |
| | CCGGCGCCAATTCCAAGGCCTGCCTGGGTCC | ATTCTCACAGATATTCTGAAACCTGTGTATTTC |
| | TATTCAGGCAGTGCTGGGGTGAGCAGCAGGC | ATTTTTGATGGAAAAGAGCTGCACACAGAATAG |
| | TCGGGCCCAGCTGACACGGCCAGAGATCCCC | TAAGTTAGCTGGAGGGAACTTATGAGCCTTTTT |
| | AGTGACTACTTTCCTGACATGGCAGAGATGG | TTTTCCCCCTCACATAAACAACAATGGAGCTTA |
| | CAGATGGAGAATCCATAAGCCCCAGTTACAC | GTGTCCATTTCATTCTCTTTGTGCTTGACTGGG |
| | CCGGGAGCTCACACTGTGGCTTCAGTCTCCA | ACCCAGATGGCTCACTGTCCCTCAGTATGTCCC |
| | AGGAGAGTGGGGAGAGCCCTGGCCCTCCGTG | TGCTCCCTCCCTGCTGAGATCTCATTGGCTGTG |
| | AAGGATTGCTTCCGCCCAAGGGGGGCCAGTG | ACGCACTGCCCTGCTCCAGCCAGGACACTACTG |
| | AACCCGAATCACTCTGCTGGATGGTGCTGGG | TCTTTCTTCCCCGTGGGAATGTGTTCTCAAAGC |
| | GGGCTGATGCAATCTGCATTCCTTCCCCTCG | CAACTCCAACAACGCTGACCTGGGCATCACTTG |
| | CACCCCTTACCCCTCGCTACCTCCCCCTTCT | GGTGGTGCTGGAGTGAGCTGTAGGCTCTGGTCC |
| | CATCCTCCCCACTCGCACCTCTCCTTCTCCC | TGCTGTTGTAGCCTGGGGTCCTAGTTGTCATTC |
| | ACACCTGGCTGACACCCACTCTTGAGTCACT | CCCTGACACAGCAGAGAGAGCAAACAACAGAAC |
| | GTCAGCTCCAAGACAGAACCGGCATCCTGGG | CAATGGCTGTAGCCACATGGTGAACAGCTAGAC |
| | TGCTTGGCAGGAGCCAAAGGAGCATGTTACA | CTCCAGAACAATAGGAGTAAATGCTTCTGCCAC |
| | GGATCTCTGGCTTCACAGATGGGGAGAGAGC | GAAGTGTATGGAGAACCTAAACCAATCTTCAGG |
| | AGTTCAGAGAATTGCGGGTTCCACATTTGCT | CAGAACTGGGGCCAGGTACCACACACAGCCCTG |
| | TGAAGTCACTCATCAGCCTTTATGTTACATT | CCCCTTTCTCAGCTGGCTGTTGCCCATGCCAGA |
| | ACAACAAAGCAGCCCAGGGGACATGGACTCA | GTCATGATCACCCATAGGATTCTCAGACCCAGG |
| | TAGGGTACCTGGTGTTTCCCCAACTGTAGGG | GCATTGTGTAGCTGGAGCTCAATGAGTCTTACG |
| | GGGATTCCGGGACAAATAAAGTTTGCCACTG | GGCCGGAAGCAGCCAATTCAGGGAACTCTGGGT |
| | GGACCCTCCCCCGAACTGTGCCCTGTCCCAC | TCTGCGTTTGCTTTGCATCTATTTGGTGAGAGA |
| | TCCTGTGACACACTCTCTGCCCACAAGAGAG | CAGTGTGAGTTCTTCCATTACAAAATTCCAATG |
| | TGGCCAACAGTGGAGGCTGAGAGTGACCACC | TTTAAAGAGCAAACAGTCAAGAAACAAGAAAAA |
| | TGCCTGCCCTCAGTTATTAAAGGCTACTGGA | AAAACCCAAGGGTGTGTCTGTGTGTGTGTGTGT |
| | GAACAAGCCTTGAGTGCGTGCTGAGAACACA | GTGCATGTGTTTATGTATGTGCAGGTACATGTT |
| | TGCCCCTAGCTGCCATCAAAGAGAATCACTT | GGGGACATGTGCATGTGCATGTTTACATGTGCA |
| | CATATGATTTTGACCATAAGCAAACTCTTCC | TAGAGAGGTCAGAAGACAACACCAGCTGTTGTT |
| | ACCTTCATTTTTTAAAATAACGGCTTTATTG | CCCCAAGTACAATCCATAGTTCAACCCCCTGTG |
| | AGATATGCATCACTTACCATGAAACTCACTC | TGTGTGTGTGTGTGTGTTTATGTGTGCATATGC |
| | TTTTAAAGTGTACAACCCAGGGTTTTCAGTG | TATGGAAGTCAAAGATTGAGTCTGGTGTCTTCA |
| | TATTCACGGAATTGTGCAACCATCACCCATC | ACTGCCCTCTACCCTATTTTCTGAAACAGAGTC |
| | ACCCCTAATTTCAGGACATTTTTATCACTCC | TCTCACTAAATCTAGACCTCACTGGTTGGGCAT |
| | AAAAAGAAACTTTGCACACATCATTCTTCTC | CCTTGTTAGCCAATGAGCTCAACTATCTGCCCG |
| | TCCCCACAGCCTCTGACAACTGCTGATCTAT | TTTGTTCTCTCTCTCTCTCTCTCTCTCTCTCTC |
| | TTTGTCTCTATGGATTTAGCAGTCATGGACA | TCTCTCTCTCTCTCTCTCTCTCTCTCTCTCTCT |
| | TTTCATATACATGGAATCATACACTATATGT | CCATAAATGAATGAATGTGTGTTTTTAAAAAGA |
| | CCTTTCATGACTGACATCTGTCACTTAGCAT | GAGTTTAAAAAAAACTAAGGTGGCATGTATCCC |
| | GATTTTATGAGATTCATCATGTTGGAGCATG | AGCTTCTCTCCACAATCCAACTGGAACGGCTCA |
| | CACCCATGCTTCCATCCTTTCTTTTTTTTTT | GGCCAGCCTCATTTCACGCAGCTCACTCTATCA |
| | TTCACAGTCTTGCTCTGTCGTGCAGGCTGAA | ACACATCTGCTGCACAGAGCATGCTTTGTGAGT |
| | GTGCAATGGCACGATTTTGGCTCACTGCAAC | GACTCAAAGATCAGAACCCTGACTTCCAATGGC |
| | CTCTGCCTCCCAGGTTCAAGCCATTCTCCTG | TTATAGCCTAAGGGTAGAGAAGTTACCTGTATT |
| | CCTCAGCCTCCCAGGTAGCTGGGACTACAGG | CTGGCAAGATACCAGGGATTGTAGGAGGGGTAG |
| | TATGTGCCACTATGCCTGGCTAATTTTTTTG | CAACCTGGGGAGGAGGGAATGCACTCTGTGTAG |
| | TATTTTTAGTAGAGATGGAGTTTCACCATGC | GAGATGCAGAAAGGATTGGAAGAGCTGGTGAGT |
| | TGGCCAGGCTGGTCTCAAACTCCTGACCTCA | ATTTGAGTTGGATGTTGGACTGATAAATGCAGG |
| | AGTGATCTGCCCGCTTCGGCCTCCCAAAGTG | GAGCATCTCACAGGTTGGGATCAGGCACACCGG |
| | CTGGGATTACAGACGTGAGCCACCACATCCT | TAGGATGTTTCATCCATCCGAGTCAAATGGAGG |
| | TTCTAAGGCTGAATAGTATTGCACTGTATGG | GCAGGTGTAGGGATTTCAGGTTAGAGGGCAGGG |
| | ATAGACCACATTTAGTTTATCTGCCTGCTGG | AAAGAAAGTAGAGAGGAGAGCCTGGGGTTGTGC |
| | CTTATGGACAATGAGTCACTCCACTTTTTGG | TGGAGTGTGCACAGAGCACTCAGCTGGCACTTT |
| | CTACTATGAATCATGCTGTTGTGAGCACTTG | GAAGAACAAAGTGGACTGTCCCTGGACGTGAGA |
| | TGTACATGTCTTTATATGGATGTCTGTTTTC | CTGAGCAGGTAAGGTGGGTTAAGAGACGGTAAG |
| | CCTTCCATTGGGTTTGCTTGGGGGTGGAATT | ATCACTACTGCAATAATCCAAAATAAGAACCTT |
| | GCTGGGCCACCTTCTTTCTCCATGAGTGGAG | TATGATCTCTAGGTGGGATAACAACCAGGGGGA |
| | CATGCCTATGCGCCCATCCCCGCATCTCCCA | GGGACTTTTAACACACAATTCAGTTCAACAGGA |
| | TGTGTGGAGGCACTGCCCAAGCTCGTCTGTA | ACTCGCACATCCTGGAGGCAACACGTGAACTGC |
| | CTCTGAGTCACAGGGCTGTGCACCATTACCG | GCAGGCTCAGCAGTCATTGTCTGTTCTGCGTGG |
| | ATCACCATCTATGGGTCAGGGACTTATCAAT | TGCTCTTCCAAGTGGCACAGTGTCTTCATCAGA |
| | GAGCAAGACATAGCCCCTGCCATCACTAACT | CCTGGTGCTCACATGACTGATCTAGTCACAGAA |
| | CACATTCTGCATCGTCCTGTGCCATCCCCAC | CAGGCCATGTATCAAGTTTTGGGAAACAGGAAG |
| | CACCCCACCTTGGTCAGGCCCAGTGTCCAGG | CAATGGGAGAAATGTATTTTATTGGTGATTAAG |
| | TGTCTTCAACTGCTCACCTTCCCCCTATTTT | TGAAGTGCAAAAGATAGGACGTGCTA |
| | GTTGCCCTGAAGTTCATCCAGACATCAGGGT | (SEQ ID NO: 29) |
| | GCCCTATTGAAAATGCTAGTTAATATGACCT | |
| | CTCTGCTCTAACCCCAATGTTGGAGTCTTGT | |
| | CATCAGTGGGATAGAGCTGGTGTGACTGCAC | |
| | CAGACCAGTCAGGTTCAACTTTTATGAAAGG | |
| | AAGTTGTGAGTTGCTTTCAGTTGCCATGGAC | |
| | CCCAAGTCGTAGGTCATGTAAGCTGAGCATG | |
| | CCCAAACGGACCAAGCATGCAACCATGGGCA | |
| | GAACCTGAGTGCTCAGACTGAGGAGCAGGGG | |
| | CTGAATTAAGAAGCAGAGCATACATGGCAGG | |
| | ATCCAGGATCCAGGAGCCAATCAGACTGAGT | |
| | TTGGCATCACTCCATGGCAGGATCCAATCAG | |
| | ATCACACCTCCCTGCAGCACCTCATTGCAAG | |
| | ATCCAATCAGACCACACCTCATTACCCTAGG | |
| | CTTATAAAATCCAGGCCAGCCGCTAGCTTGG | |
| | GGAGGCAGATTTGAGTGTTTTTTTTTTTCTG | |
| | TCTCCTTGCCAGACTACCAGCAAAAAAGGTT | |
| | TTCTTTTCTCAAAAGCCGGTGTCATGGTATT | |
| | GGCCTCTGTGCACATTGGGCAGTGAGCCCAC | |
| | TGATTGCTCAGTAACATGGGCACACTCTGGG | |
| | GCCCACACAAGCCAGGAATGATGTGGCCTTT | |
| | ACCTGCTGCTCCAGCTGCATCTGAGCCCAGT | |
| | ATCCCCTGAACACAAACCCCCACCTGCATGG | |
| | AGCTGCATGCGGTTCTCGGGTACCTCCTGGC | |
| | TATGTTCAGCTCCTGTAGATTCCTTCAGATC | |
| | CACTCCTTCCCATTTCCTCATCCAACTGCCC | |
| | AGCAGAGTGCCTACTATGCGCCACACACTGG | |
| | GATTCAGCAGTAAACGACACAAACATGATCC | |
| | CCACCCTTATCCTTCTCCCAGGACTCTTATT | |
| | AATCTAAGGCTCACCTCCCTTCTTGTAACTT | |
| | CCATGAACTCATATGCTCCCTCTCAGCTCAG | |
| | GGACGTTGCTGGAGGAAGCAAGAGAGCAGCA | |
| | GATGAACCCTTATGTTCAGGAGGCAGATGGA | |
| | GCTCATTCAAAGCCCACCTTGGCCTCTTCTT | |
| | AACCCGAAGATTTTAGCAAGTCATATAACCT | |
| | TTGAACTGCAACTCCCTGGATTGTGGAATGC | |
| | CCAAAGTGTGCTGAGCGTGAAGTAAATAATG | |
| | CACATTCTGCATCGTCCTGTGCCATCCCCAC | |
| | CACCCCACCTTGGTCAGGCCCAGTGTCCAGG | |
| | TGTCTTCAACTGCTCACCTTCCCCCTATTTT | |
| | GTTGCCCTGAAGTTCATCCAGACATCAGGGT | |
| | GCCCTATTGAAAATGCTAGTTAATATGACCT | |
| | CTCTGCTCTAACCCCAATGTTGGAGTCTTGT | |
| | CATCAGTGGGATAGAGCTGGTGTGACTGCAC | |
| | CAGACCAGTCAGGTTCAACTTTTATGAAAGG | |
| | AAGTTGTGAGTTGCTTTCAGTTGCCATGGAC | |
| | CCCAAGTCGTAGGTCATGTAAGCTGAGCATG | |
| | CCCAAACGGACCAAGCATGCAACCATGGGCA | |
| | GAACCTGAGTGCTCAGACTGAGGAGCAGGGG | |
| | CTGAATTAAGAAGCAGAGCATACATGGCAGG | |
| | ATCCAGGATCCAGGAGCCAATCAGACTGAGT | |
| | TTGGCATCACTCCATGGCAGGATCCAATCAG | |
| | ATCACACCTCCCTGCAGCACCTCATTGCAAG | |
| | ATCCAATCAGACCACACCTCATTACCCTAGG | |
| | CTTATAAAATCCAGGCCAGCCGCTAGCTTGG | |
| | GGAGGCAGATTTGAGTGTTTTTTTTTTTCTG | |
| | TCTCCTTGCCAGACTACCAGCAAAAAAGGTT | |
| | TTCTTTTCTCAAAAGCCGGTGTCATGGTATT | |
| | GGCCTCTGTGCACATTGGGCAGTGAGCCCAC | |
| | TGATTGCTCAGTAACATGGGCACACTCTGGG | |
| | GCCCACACAAGCCAGGAATGATGTGGCCTTT | |
| | ACCTGCTGCTCCAGCTGCATCTGAGCCCAGT | |
| | ATCCCCTGAACACAAACCCCCACCTGCATGG | |
| | AGCTGCATGCGGTTCTCGGGTACCTCCTGGC | |
| | TATGTTCAGCTCCTGTAGATTCCTTCAGATC | |
| | CACTCCTTCCCATTTCCTCATCCAACTGCCC | |
| | AGCAGAGTGCCTACTATGCGCCACACACTGG | |
| | GATTCAGCAGTAAACGACACAAACATGATCC | |
| | CCACCCTTATCCTTCTCCCAGGACTCTTATT | |
| | AATCTAAGGCTCACCTCCCTTCTTGTAACTT | |
| | CCATGAACTCATATGCTCCCTCTCAGCTCAG | |
| | GGACGTTGCTGGAGGAAGCAAGAGAGCAGCA | |
| | GATGAACCCTTATGTTCAGGAGGCAGATGGA | |
| | GCTCATTCAAAGCCCACCTTGGCCTCTTCTT | |
| | AACCCGAAGATTTTAGCAAGTCATATAACCT | |
| | TTGAACTGCAACTCCCTGGATTGTGGAATGC | |
| | CCAAAGTGTGCTGAGCGTGAAGTAAATAATG | |
| | CAAGTGTAAAGTGTGCGGCATGGTCCTGGTT | |
| | CATCTCAGGAGGCCGTTAGGAAACTAGCACT | |
| | TATTTTTGCCAGGGCTTGAGCATAGAACATA | |
| | CTAATTTCCCCAATGGCATTATCACATTGTA | |
| | TTACTTTTTATTTACATGTTCTTTCTCCCCT | |
| | ACCAATCTCAGAGAATCTCAAGGGCAGCAAT | |
| | GATTAATTATTAATTTTGGAATCCTTGGTTC | |
| | CTGGCACATTCCTTGAAAATAAATCATTGGC | |
| | TTACTTTCCACTGATTCTCTTAATTACCCCT | |
| | GAGAGGCAGAGATTGGAATTATACTATGCTG | |
| | AGCAGCTCAATGTTTTCCCAGTAACAGCAGG | |
| | AAAATCCCAATGCACAGAGAAGGAACCTGAA | |
| | TGACTTAGGTGGGACACACCAGGACAGACAC | |
| | CCGTGGTGATGACATTCTGTGCCCTTCATCC | |
| | CACAGAGTGGTCTGTCTTCACAGTGGTCTCC | |
| | CCTCACCACACTGAGCCCTCAAACTTCCTCT | |
| | TTCCGCTGACCAAAGTGCACCCAGGCCTGCT | |
| | TGTCCATTCAGACAGATGCCAGGGCCCTCTG | |
| | CACTCCATCTGACCTCTGCAATATGCCGGTT | |
| | CCTAATAAGGGAGCAGGATCCAGGTCCAGTT | |
| | GTTCACACTTCTAATTTCATACCGGCAGCCT | |
| | CAGTAAAGTTCTGCCATCAGGCTAAGGCCCC | |
| | ACTGATCGTCGACCTTTTCTGCATAAAGATT | |
| | CACCTCCAGGGCTCTTAGAAAATACTGCTGC | |
| | CTGGCTACCACCCCATCCTTAGTGTGACATA | |
| | GGGTTTTTTTTTCTTCTTCTTCTGTTTTTTG | |
| | TTTTTTTTAGAATAATTAGGCAGCTCTGTTG | |
| | CCCAGGCTGGAGTGCAGTGGCATGATCTCAG | |
| | CTCACTGCAACCTCTGCCTCCTGGTTCAAGC | |
| | AATTCTCCTACCTCAGCCTCTTGAGTACCTA | |
| | GGACTATAGGCACACGCCACCATGCCCGGCT | |
| | AATTTTTTGTATTTTTAGTAGAGACGGGGTT | |
| | TCACCAGGTTAGCCAGGATGGTCTCAATCTC | |
| | CTGACCTTGTGATCCGCCCACCTCAGCCTCC | |
| | CAAAGTGCTGGGATTACAGACGTGAGGCACC | |
| | ACACCTGGCCTGCCCCGGGTTGTTTTTTTTT | |
| | TTAAAGCTCCCCAGGGATTTGTAAGTGCATA | |
| | CCAAAGACTGGGAACCCCTGGCTTAGCTCAC | |
| | AGAGCAAAGAGCCTTTTGAGGGTTCCCCTCG | |
| | ACAGTTGCTCCCTCACCTCCAGCTGTGGGGC | |
| | CACACAGAGCGCTGGGCCATTGTGGTGTTAG | |
| | AGACCAGAGTTAAAGGGACTCCATCTGTAAT | |
| | ATCCAGGACAAATGGGCTGGCAGGTGCTGCT | |
| | CAAACCCTTACACACAGATAGTATTTGGGGA | |
| | GGTGAGGTCAATTCCCCCATTATGGAACGCT | |
| | GCGGTTTTAAAAGCAAGCAAACAAACAAAAA | |
| | CAGGAAAAAAGTGAGCTTTTTAAAACTAAGG | |
| | TAAAATTTGTCCTCAACTTCCTGGCCTTGAT | |
| | TGGGCTCTGCTACTAGAGCGGCAGAAGCAAC | |
| | TCACTTCCCTGCTTCCACGGACCTGTTTCAT | |
| | GTAATGCATTTTGCAGAGATTTGAAGACAGG | |
| | GTCCTTGACTTGGGCAGCTAACAGCCTGAGG | |
| | CTAGAGGCAGCCACCCCTGAACAGTGAACAA | |
| | TTCTGCAAGGCGCCTGGCAATAGTACTATGC | |
| | GGGGAGGGGGTAGGAACAAGGTGCTGCAGGG | |
| | CGGGGTGGAGGAGGAAATGAATTCTGCCTGG | |
| | GAGAAGCGGGAGTGCGTATTTGAGTGGGGTC | |
| | TGGAGCAGGTGCATGCAAAGAAGCACCTCAA | |
| | AGGCACGGGCAGGTGTGTGCAGGCGTGGGCA | |
| | GGCGTGGGCAGGCGTGGGAAGGCGTGGGCAG | |
| | GCGTGGGCAGGTGTGGGCAGGCGTGGGCAGG | |
| | CGTGGGCAGGCGTGGGCAGGTGTGGGCAGGT | |
| | GTGGGCAGGCATGTGGGCACGGCACAGGGCT | |
| | TGTCCAGGCCAGATGCCATTAAGCACAGGTA | |
| | TCTGTGGTGGGCAGGGGACACAGTGGAAGCA | |
| | GATAGAGAAGGTTTGCTGGGGTCCCATGGAG | |
| | GGGCGCCTTGTAGGCCATGGTCACTCTAGGC | |
| | TGATGCAAGGTGCTCAAGGTTGAAGGCAGAG | |
| | GTGACTGACCTGTGCTTGAGAGAGGGTAGGG | |
| | AAGAGAAGCTGCCGGACTTGAGGGGCTGAAA | |
| | TTGTCCTGTAATAGTCCAGGTCAGGAGTGTT | |
| | AATGATGCCCCAGCTCGGGCAGTGACTACGG | |
| | CAAGGAGAGTTTAACATGTGGTTCAGTTCAG | |
| | CAGACATGGGGAACTCACTATGTGTGAAGCA | |
| | GGACACATCACGGAGGCAGCCCTCAAATGCT | |
| | TGAAGACAGTAATCCTGCCCCTGTGCTGTGG | |
| | CGGGTTCTTTAAGGGGTGTGACTTCCTCATC | |
| | AGACCCATTGCTCTCACACCTAATGATGCTG | |
| | CCATGTGGCAGGGCTGTGGGCAGAGCCATGC | |
| | CCTAGCAGGGGAAGTGGAGGACAGCGGCGGG | |
| | GAGGGAGTGTGGGCAGGGCTTTCCTGCCCTC | |
| | TGGGTCCTCTCCTCTCTTTCGTGGCAGGGCC | |
| | TTGAGGTCCATTCGCTGGGCTGCACAGAAGG | |
| | AGGACTCCAGAGCCCCCCTTGGGTTCAGGAT | |
| | TTTATACACGCAGCATTCCAGACAGATGGAC | |
| | CCGTGTATTGACAATGAAAGCATGGGAGAAC | |
| | TGTATTTCTTTGGTGATTAAAGTAAATGCAA | |
| | AAGTTATGATGC (SEQ ID NO: 28) | |

In some embodiments, a GJB2 GRE (e.g., a GJB2 enhancer) sequences can be identified from the regional sequence listed in Table 2. In some embodiments, a GJB2 GRE (e.g., a GJB2 enhancer) comprises at least 200, at least 250, at least 300, at least 350, at least 400, at least 450, at least 500, at least 550, at least 600, at least 650, at least 700, at least 750, at least 800, at least 850, at least 900 at least 1000, at least 1100, at least 1200, at least 1300, at least 1400, at least 1500, at least 1600, at least 1700, at least 1800, at least 1900, at least 2000, at least 2100, at least 2200, at least 2300, at least 2400, at least 2500, at least 2600, at least 2700, at least 2800, at least 2800, at least 2900, at least 3000, at least 3100, at least 3200, at least 3300, at least 3400, at least 3500, at least 3600, at least 3700, at least 3800, at least 3900, at least 4000, at least 4100, at least 4200, at least 4200, at least 4400, at least 4500, at least 4600, at least 4700, at least 4800, at least 4900, at least 5000, or more consecutive nucleotides in any of the regional sequences described in Table 2 (e.g., human GJB2 regions A-M or mouse Gjb2 regions A-M). In some embodiments, a GJB2 GRE (e.g., a GJB2 enhancer) is identified with the transcriptionally active regions of the GJB2 gene (e.g., regions A and/or B). In some embodiments, the GJB2 GRE (e.g., a GJB2 enhancer) comprises at least 200, at least 250, at least 300, at least 350, at least 400, at least 450, at least 500, at least 550, at least 600, at least 650, at least 700, at least 750, at least 800, at least 850, at least 900 at least 1000, at least 1100, at least 1200, at least 1300, at least 1400, at least 1500, at least 1600, at least 1700, at least 1800, at least 1900, at least 2000, at least 2100, at least 2200, at least 2300, at least 2400, at least 2500, at least 2600, at least 2700, at least 2800, at least 2800, at least 2900, at least 3000, at least 3100, at least 3200, at least 3300, at least 3400, at least 3500, at least 3600, at least 3700, at least 3800, at least 3900, at least 4000, at least 4100, at least 4200, at least 4200, at least 4400, at least 4500, at least 4600, at least 4700, at least 4800, at least 4900, at least 5000, or more consecutive nucleotides in within regions A and/or B. In some embodiments, the GJB2 GRE (e.g., a GJB2 enhancer) comprises at least 200, at least 250, at least 300, at least 350, at least 400, at least 450, at least 500, at least 550, at least 600, at least 650, at least 700, at least 750, at least 800, at least 850, at least 900 at least 1000, at least 1100, at least 1200, at least 1300, at least 1400, at least 1500, at least 1600, at least 1700, at least 1800, at least 1900, at least 2000, at least 2100, at least 2200, at least 2300, at least 2400, at least 2500, at least 2600, at least 2700, at least 2800, at least 2800, at least 2900, at least 3000, at least 3100, at least 3200, at least 3300, at least 3400, at least 3500, at least 3600, at least 3700, at least 3800, at least 3900, at least 4000, at least 4100, at least 4200, at least 4200, at least 4400, at least 4500, at least 4600, at least 4700, at least 4800, at least 4900, at least 5000, or more consecutive nucleotides in within regions C-M. In some embodiments, the GJB2 GRE (e.g., a GJB2 enhancer) comprises nucleotide sequences out of the regions listed in Table 3.

In some embodiments, a GJB2 GRE (e.g., a GJB2 enhancer) is located on the sense strand of the GJB2 coding sequence in the genome. In some embodiments, GJB2 GRE (e.g., a GJB2 enhancer) is located on the reverse complement strand of the GJB2 coding sequence in the genome. It is within the skill of one in the art to select the appropriate sequence (e.g., GRE sequence on the sense strand, or GRE sequences on the reverse complement strand) when designing a vector using the enhancer sequences as described herein.

In some embodiments, a GJB2 GRE (e.g., a GJB2 enhancer) comprises at least 200, at least 250, at least 300, at least 350, at least 400, at least 450, at least 500, at least 550, at least 600, at least 650, at least 700, at least 750, at least 800, at least 850, at least 900 at least 1000, at least 1100, at least 1200, at least 1300, at least 1400, at least 1500, at least 1600, at least 1700, at least 1800, at least 1900, at least 2000, at least 2100, at least 2200, at least 2300, at least 2400, at least 2500, at least 2600, at least 2700, at least 2800, at least 2800, at least 2900, at least 3000, at least 3100, at least 3200, at least 3300, at least 3400, at least 3500, at least 3600, at least 3700, at least 3800, at least 3900, at least 4000, at least 4100, at least 4200, at least 4200, at least 4400, at least 4500, at least 4600, at least 4700, at least 4800, at least 4900, at least 5000, or more nucleotides. In some embodiments, a GJB2 GRE (e.g., a GJB2 enhancer) comprises 200-500 nucleotides or any number of nucleotides in between, 300-600 nucleotides or any number of nucleotides in between, 400-700 nucleotides or any number of nucleotides in between, 500-800 nucleotides or any number of nucleotides in between, 600-900 nucleotides or any number of nucleotides in between, 700-1000 nucleotides or any number of nucleotides in between, 1000-1500 nucleotides or any number of nucleotides in between, 1500-2000 nucleotides or any number of nucleotides in between. In some embodiments, a GJB2 GRE (e.g., a GJB2 enhancer) comprises 700 nucleotides.

In some embodiments, the GJB2 GRE is a human GJB2 enhancer. In some embodiments, the GJB2 GRE (e.g., a human GJB2 enhancer) comprises nucleotide sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to any one of the GRE sequences as listed in Table 3.

TABLE 3
Human GJB2 Enhancer Sequences

| | Sense strand | Reverse Complement strand |
|---|---|---|
| hGJB2 GRE1 | TCATCCATGTCCCTACAAAGGACAT | AAGAGAGCACTTGGGAAGAGCCCCCG |
| | GAACTCATCATTTTTTATGGCTGCA | AGGGCAGCCGGGGCTTGCCGCCTCAC |
| | TAAGTCGTTCTTTCAAACACCCTGC | CCTTTTGGTTTCACATCCCAGAAATC |
| | AGTCAGCTTCTCCTCACGAGAAACC | AGTAAGGCAGGAATTGGAGGCTGCTT |
| | ACATGAAAGCCCTCGGGGAAATGCC | CTTGCCTTAGCAACTCGGTGACCTTA |
| | TCTCGGGATCTACTTTTCTTTGTGT | GGCAGAACAGTTCAGCCTTCTGAGTG |
| | GTATCCTACTTAGCCTATCGGTTTC | TCCTTCCTCTTCTGTAAGGGGAGCGT |
| | TGCTTCCTGTGGGGCTACAGCCGTC | AAACCGTCCTCCATGCAGAACGTGTA |
| | TCGTCTTTTTCTGCTGGCTCCTTTG | CTGTGCCTGGCACAGCACTGGGGCAT |
| | CTCTGTTCTCCAGTGGCTATCTTCT | TAGGATCTCCAAATTAAAGGCTCACT |
| | TTCTCCTTTCTTTCAAATGTTCTCC | CTGCGGGATGGAGGCAGCCACAGCTG |
| | CTTATCTTCTCTGATACAGACAGAA | GAAGAAGGAACATTTGGGGCCAGAAG |
| | GGTCAGGAGCCACGCCCATTACACT | TCCCCCTACCTCCGTCCTAAGAGAGA |
| | GACAGAACCCGATGTCCTGATGCGC | AGATGGGAATAACGACCCTCGCTGAA |
| | TCTGTGCCTCCCAGATTTGGATGTG | ATGATTGCTCTCTGGCCAGCTCGCCT |
| | GATGCGAGGCGAGCTGGCCAGAGAG | CGCATCCACATCCAAATCTGGGAGGC |
| | CAATCATTTCAGCGAGGGTCGTTAT | ACAGAGCGCATCAGGACATCGGGTTC |
| | TCCCATCTTCTCTCTTAGGACGGAG | TGTCAGTGTAATGGGCGTGGCTCCTG |
| | GTAGGGGGACTTCTGGCCCCAAATG | ACCTTCTGTCTGTATCAGAGAAGATA |
| | TTCCTTCTTCCAGCTGTGGCTGCCT | AGGGAGAACATTTGAAAGAAAGGAGA |
| | CCATCCCGCAGAGTGAGCCTTTAAT | AAGAAGATAGCCACTGGAGAACAGAG |
| | TTGGAGATCCTAATGCCCCAGTGCT | CAAAGGAGCCAGCAGAAAAAGACGAG |
| | GTGCCAGGCACAGTACACGTTCTGC | ACGGCTGTAGCCCCACAGGAAGCAGA |
| | ATGGAGGACGGTTTACGCTCCCCTT | AACCGATAGGCTAAGTAGGATACACA |
| | ACAGAAGAGGAAGGACACTCAGAAG | CAAAGAAAAGTAGATCCCGAGAGGCA |
| | GCTGAACTGTTCTGCCTAAGGTCAC | TTTCCCCGAGGGCTTTCATGTGGTTT |
| | CGAGTTGCTAAGGCAAGAAGCAGCC | CTCGTGAGGAGAAGCTGACTGCAGGG |
| | TCCAATTCCTGCCTTACTGATTTCT | TGTTTGAAAGAACGACTTATGCAGCC |
| | GGGATGTGAAACCAAAAGGGTGAGG | ATAAAAAATGATGAGTTCATGTCCTT |
| | CGGCAAGCCCCGGCTGCCCTCGGGG | TGTAGGGACATGGATGA |
| | GCTCTTCCCAAGTGCTCTCTT | (SEQ ID NO: 56) |
| | (SEQ ID NO: 55) | |
| hGJB2 GRE2 | CCATGATATGTTAAGAAAAGCAAAG | ATATAATCTGTTTTTTCCTATACATA |
| | TGTGGAATAGTAGGTAAAATATTCT | CAAACCTACCATAAGGCTTAATGGTA |
| | ATCTTATGTGCAAAAGGGGAAATAA | AGAGATTAACAATAAAGAATAATAAA |
| | AAGTCATCAATATTCATGTAGATTC | ACAACACTTATAACAATGTATAACAA |
| | AATTCACATATAGATTCATATCACA | TATATTGTAATATAAGTTTTTGGATG |
| | TTCCTATATATATAGAAATTCTGGA | CAGTCTCTCTCTCAAAATGCTATCAT |
| | AAGACACAAAATAAATTAATAAAAG | ATTTTCCAACTGTGGTTGACTACAGG |
| | TTGTTACTTCATTGTAGTTTTTAAA | TAACTGGAACCACAAAAATGAAACAG |
| | GTTTTTTGAGTCTTAAGACTTACTT | TGGATAAGAGGGCGACTCCTGTACCA |
| | TCCACTTCTGTAGAAAGGAATTACA | AAGAAAAAAATAGAGTGTTGCAGCTG |
| | AATCCTTTCTTTATAGAGCTATGTG | TAACATAGTTGAATGACTGAGTTAGA |
| | ATGAAATAAACATAAAGCATTTGGC | CTGCATAACTGACACACAAAACCACA |
| | ACACTTCAGGATAGCAACTTGTGGA | TAAATATAAATGAAGGAATCTCTGGG |
| | TTAATGATTAACACAGTCACCTTTG | TGTAATCTGGTGCAAAGGTGACTGTG |
| | CACCAGATTACACCCAGAGATTCCT | TTAATCATTAATCCACAAGTTGCTAT |
| | TCATTTATATTTATGTGGTTTTGTG | CCTGAAGTGTGCCAAATGCTTTATGT |
| | TGTCAGTTATGCAGTCTAACTCAGT | TTATTTCATCACATAGCTCTATAAAG |
| | CATTCAACTATGTTACAGCTGCAAC | AAAGGATTTGTAATTCCTTTCTACAG |
| | ACTCTATTTTTTTCTTTGGTACAGG | AAGTGGAAAGTAAGTCTTAAGACTCA |
| | AGTCGCCCTCTTATCCACTGTTTCA | AAAAACTTTAAAAACTACAATGAAGT |
| | TTTTTGTGGTTCCAGTTACCTGTAG | AACAACTTTTATTAATTTATTTTGTG |
| | TCAACCACAGTTGGAAAATATGATA | TCTTTCCAGAATTTCTATATATATAG |
| | GCATTTTGAGAGAGAGACTGCATCC | GAATGTGATATGAATCTATATGTGAA |
| | AAAAACTTATATTACAATATATTGT | TTGAATCTACATGAATATTGATGACT |
| | TATACATTGTTATAAGTGTTGTTTT | TTTATTTCCCCTTTTGCACATAAGAT |
| | ATTATTCTTTATTGTTAATCTCTTA | AGAATATTTTACCTACTATTCCACAC |
| | CCATTAAGCCTTATGGTAGGTTTGT | TTTGCTTTTCTTAACATATCATGG |
| | ATGTATAGGAAAAAACAGATTATAT | (SEQ ID NO: 38) |
| | (SEQ ID NO: 37) | |
| hGJB2 GRE3 | GCAGAGACCTACAGACAGAAGTACA | TAGGATTGACAAGGGCAATAGAGCGA |
| | TTTTACACTGGATCCAGGACACACA | TGACTCCCTGGCTGTGTTGTATTTGA |
| | TCAGTCTGAAAACACACACATGAAC | TGGACGGCAGTAGCTTTTCACAAAAT |
| | CAAACGTTTCCTAAAGCATTACTTA | GCTCATTTGGATGTTTCAAATTAAAA |
| | TCCTTGCTAATAGCAACACATTCTC | CGTTTCACTTTCTAGAACCAATTACG |
| | ATATTCTTTTATACTTCATTTAATT | TGGTCAGTTTAGCTCCTGAGGTCCCA |
| | TCATATAAAAAAGAAAAGGAAAGGA | GTCAGAGGGGTATTCTGTAGCTTGCA |
| | AAGAAATCTATTTCTCAGCCCATTA | AAGCCTCTCTTTGGGGACTGGACATG |
| | ATAAGGTCAGGAGCAGCAACACCAG | GAGTCTGTGGTCTTAGAATTCAGAAC |
| | ACTAGAAGAAAAGCTTACCTATAGA | CGGGAGAATGTGTTAGCCACTCATCT |
| | TTTTTCTGCCACCTCTTGAGTGCGT | AAGCTATTCCTTAAACGCTTTCAGAG |
| | CCAGCTTTCCGACAAGTCTCAGTGC | CCATCTCCACTGTGGGGAAAGAAGTT |
| | CATCTACTGTGCGCTCTGGGTATTG | CTTTGTGTTCTCTGACTTAGTCTCAT |
| | CAATTGCTTTTTTTTTTTTTTTTTT | TCTAAAAAAAAAAAAAAAAAAAAAAA |
| | TTTTTTTTTAGAATGAGACTAAGTC | AAAAGCAATTGCAATACCCAGAGCGC |
| | AGAGAACACAAAGAACTTCTTTCCC | ACAGTAGATGGCACTGAGACTTGTCG |
| | CACAGTGGAGATGGCTCTGAAAGCG | GAAAGCTGGACGCACTCAAGAGGTGG |
| | TTTAAGGAATAGCTTAGATGAGTGG | CAGAAAAATCTATAGGTAAGCTTTTC |
| | CTAACACATTCTCCCGGTTCTGAAT | TTCTAGTCTGGTGTTGCTGCTCCTGA |
| | TCTAAGACCACAGACTCCATGTCCA | CCTTATTAATGGGCTGAGAAATAGAT |
| | GTCCCCAAAGAGAGGCTTTGCAAGC | TTCTTTCCTTTCCTTTTCTTTTTTAT |
| | TACAGAATACCCCTCTGACTGGGAC | ATGAAATTAAATGAAGTATAAAAGAA |
| | CTCAGGAGCTAAACTGACCACGTAA | TATGAGAATGTGTTGCTATTAGCAAG |
| | TTGGTTCTAGAAAGTGAAACGTTTT | GATAAGTAATGCTTTAGGAAACGTTT |
| | AATTTGAAACATCCAAATGAGCATT | GGTTCATGTGTGTGTTTTCAGACTGA |
| | TTGTGAAAAGCTACTGCCGTCCATC | TGTGTGTCCTGGATCCAGTGTAAAAT |
| | AAATACAACACAGCCAGGGAGTCAT | GTACTTCTGTCTGTAGGTCTCTGC |
| | CGCTCTATTGCCCTTGTCAATCCTA | (SEQ ID NO: 40) |
| | (SEQ ID NO: 39) | |
| hGJB2 GRE4 | CTTGCTTACCCAGACTCAGAGAAGT | GACACTGCAATCATGAACACTGTGAA |
| | CTCCCTGTTCTGTCCTAGCTAGTGA | GACAGTCTTCTCCGTGGGCCGGGACA |
| | TTCCTGTGTTGTGTGCATTCGTCTT | CAAAGCAGTCCACAGTGTTGGGACAA |
| | TTCCAGAGCAAACCGCCCAGAGTAG | GGCCAGGCGTTGCACTTCACCAGCCG |
| | AAGATGGATTGGGGCACGCTGCAGA | CTGCATGGAGAAGCCGTCGTACATGA |
| | CGATCCTGGGGGGTGTGAACAAACA | CATAGAAGACGTACATGAAGGCGGCT |
| | CTCCACCAGCATTGGAAAGATCTGG | TCGAAGATGACCCGGAAGAAGATGCT |
| | CTCACCGTCCTCTTCATTTTTCGCA | GCTTGTGTAGGTCCACCACAGGGAGC |
| | TTATGATCCTCGTTGTGGCTGCAAA | CTTCGATGCGGACCTTCTGGGTTTTG |
| | GGAGGTGTGGGGAGATGAGCAGGCC | ATCTCCTCGATGTCCTTAAATTCACT |
| | GACTTTGTCTGCAACACCCTGCAGC | CTTTATCTCCCCCTTGATGAACTTCC |
| | CAGGCTGCAAGAACGTGTGCTACGA | TCTTCTTCTCATGTCTCCGGTAGGCC |
| | TCACTACTTCCCCATCTCCCACATC | ACGTGCATGGCCACTAGGAGCGCTGG |
| | CGGCTATGGGCCCTGCAGCTGATCT | CGTGGACACGAAGATCAGCTGCAGGG |
| | TCGTGTCCACGCCAGCGCTCCTAGT | CCCATAGCCGGATGTGGGAGATGGGG |
| | GGCCATGCACGTGGCCTACCGGAGA | AAGTAGTGATCGTAGCACACGTTCTT |
| | CATGAGAAGAAGAGGAAGTTCATCA | GCAGCCTGGCTGCAGGGTGTTGCAGA |
| | AGGGGGAGATAAAGAGTGAATTTAA | CAAAGTCGGCCTGCTCATCTCCCCAC |
| | GGACATCGAGGAGATCAAAACCCAG | ACCTCCTTTGCAGCCACAACGAGGAT |
| | AAGGTCCGCATCGAAGGCTCCCTGT | CATAATGCGAAAAATGAAGAGGACGG |
| | GGTGGACCTACACAAGCAGCATCTT | TGAGCCAGATCTTTCCAATGCTGGTG |
| | CTTCCGGGTCATCTTCGAAGCCGCC | GAGTGTTTGTTCACACCCCCCAGGAT |
| | TTCATGTACGTCTTCTATGTCATGT | CGTCTGCAGCGTGCCCCAATCCATCT |
| | ACGACGGCTTCTCCATGCAGCGGCT | TCTACTCTGGGCGGTTTGCTCTGGAA |
| | GGTGAAGTGCAACGCCTGGCCTTGT | AAGACGAATGCACACAACACAGGAAT |
| | CCCAACACTGTGGACTGCTTTGTGT | CACTAGCTAGGACAGAACAGGGAGAC |
| | CCCGGCCCACGGAGAAGACTGTCTT | TTCTCTGAGTCTGGGTAAGCAAG |
| | CACAGTGTTCATGATTGCAGTGTC | (SEQ ID NO: 58) |
| | (SEQ ID NO: 57) | |
| hGJB2 GRE5 | ATCCATTATTTGATTAGCCATTTCA | GTAGTGTATGTTTGTGTGAATTTTTG |
| | AAAACACATTTACGGAGATCTTCAT | TTTTTAATTTTTTATGAGTGCCCTAA |
| | CTGGGCAGAGCATTATTCCAGGCCT | CAAAGATTACAAATTGGGAATACAAA |
| | CTGAAGAACCAAAGATGATTTTGAA | CTCCAGAGCAATGGAGACAGTGACAC |
| | AGGAGGTCACAGTGCAGACAGCAGG | TTTTGTGGAGGGGTACATGTGGCTGT |
| | TGTGTATATAAGGTGGCTACTTTAC | TCGGGTGGTTATTAACACAGGCTGCT |
| | AAAACAGGATATGGCAAGCTGGACA | GCCCCTGCCCTGCAATGGGAATCCCC |
| | TGACAGGCACAGCAAAGTCTCTGAA | AGGGCATTGGAGGATTCAACCTCTTG |
| | CAGAGTTCGGGGCATGAAATTGTTT | CAGTTACCTCTTGTAAGACAGCAGAT |
| | CTTTTGGGGGTCTTCAGGAACAATT | GGCAGCAGAGAGAGGCTTTGCACATC |
| | TCATGAAAGCTAAATCATGAAAGAT | CCTGCAGGTTCTAGTTTGCACAAAGG |
| | AGCAGGCTTTTGCCAGGAAAAAAAA | GCTTCTGAGAGACCTATCAACCAATT |
| | AAACAAGACTAGTGATTAGTTTGGC | ATAACATCAAGTGGCAAAAAGAGTCC |
| | GTTTTCGGTTTCTTTGAGAAGCGAA | TTGATAAGTTATTTCGCTTCTCAAAG |
| | ATAACTTATCAAGGACTCTTTTTGC | AAACCGAAAACGCCAAACTAATCACT |
| | CACTTGATGTTATAATTGGTTGATA | AGTCTTGTTTTTTTTTTTCCTGGCAA |
| | GGTCTCTCAGAAGCCCTTTGTGCAA | AAGCCTGCTATCTTTCATGATTTAGC |
| | ACTAGAACCTGCAGGGATGTGCAAA | TTTCATGAAATTGTTCCTGAAGACCC |
| | GCCTCTCTCTGCTGCCATCTGCTGT | CCAAAAGAAACAATTTCATGCCCCGA |
| | CTTACAAGAGGTAACTGCAAGAGGT | ACTCTGTTCAGAGACTTTGCTGTGCC |
| | TGAATCCTCCAATGCCCTGGGGATT | TGTCATGTCCAGCTTGCCATATCCTG |
| | CCCATTGCAGGGCAGGGGCAGCAGC | TTTTGTAAAGTAGCCACCTTATATAC |
| | CTGTGTTAATAACCACCCGAACAGC | ACACCTGCTGTCTGCACTGTGACCTC |
| | CACATGTACCCCTCCACAAAAGTGT | CTTTCAAAATCATCTTTGGTTCTTCA |
| | CACTGTCTCCATTGCTCTGGAGTTT | GAGGCCTGGAATAATGCTCTGCCCAG |
| | GTATTCCCAATTTGTAATCTTTGTT | ATGAAGATCTCCGTAAATGTGTTTTT |
| | AGGGCACTCATAAAAAATTAAAAAC | GAAATGGCTAATCAAATAATGGAT |
| | AAAAATTCACACAAACATACACTAC | (SEQ ID NO: 42) |
| | (SEQ ID NO: 41) | |
| hGJB2 GRE7 | GCTAATTGGGTCAGGATTTGAAAGA | ATCTTAGCTCCAACATGTCATTATTC |
| | CCTTAGCTTTGTGTGACCTTCAATT | CTTCCTCACTGAGGACTTTTCTGCTT |
| | TTATCATTCAGCTTGAATATGTGCC | CCTAATTGGTTGTTGAAGATGAGGCC |
| | CCAGAAAACCTTTATGTAATTCCCT | CCCATGCTCTTTTAAGAAAACCTGTT |
| | AATATTTCAGTAACCAGCATGCAAC | GTGCCCCAGGCTTGGCTGTGATGGGC |
| | ATACGAGAAGCACATTCTTTGTTTT | ACTGACTCATACAGAAGTAGAAAGGC |
| | TAGAATGGTATCTGGCTGATGACTT | CTGCTGAGTCATCAACACTCGTGCGA |
| | TCACAACAGCTCACATGAGAGGGAA | CGCCCTCGCATTTTCATTAATGATGG |
| | GTATTTTAGCAATCGGACTGAAGGA | CCTCCCTGCCACACGTGAATCACTCC |
| | AAATCCAAAAACTCCACCATTGCAG | AGCCCGAGATCTGAAACCAGGACACA |
| | GGTCAACAGTGCACGTGTTTGAATT | CCCCAGGGGCGAGGTGACGCTGAGTG |
| | CTGAAAGACGTAAGCCAAGGCAAAT | AGCCCAGCTGTGTCCCTTTCATGAGA |
| | AGAAGGAAATGATCTTCCACTAATC | ACTCAGAGCACAGGGCTCTGTGTGCA |
| | CCGGCATTTACTTCCTCCTCTCTGG | TGGCCGTCCCCTCCAGAGAGGAGGAA |
| | AGGGGACGGCCATGCACACAGAGCC | GTAAATGCCGGGATTAGTGGAAGATC |
| | CTGTGCTCTGAGTTCTCATGAAAGG | ATTTCCTTCTATTTGCCTTGGCTTAC |
| | GACACAGCTGGGCTCACTCAGCGTC | GTCTTTCAGAATTCAAACACGTGCAC |
| | ACCTCGCCCCTGGGGTGTGTCCTGG | TGTTGACCCTGCAATGGTGGAGTTTT |
| | TTTCAGATCTCGGGCTGGAGTGATT | TGGATTTTCCTTCAGTCCGATTGCTA |
| | CACGTGTGGCAGGGAGGCCATCATT | AAATACTTCCCTCTCATGTGAGCTGT |
| | AATGAAAATGCGAGGGCGTCGCACG | TGTGAAAGTCATCAGCCAGATACCAT |
| | AGTGTTGATGACTCAGCAGGCCTTT | TCTAAAAACAAAGAATGTGCTTCTCG |
| | CTACTTCTGTATGAGTCAGTGCCCA | TATGTTGCATGCTGGTTACTGAAATA |
| | TCACAGCCAAGCCTGGGGCACAACA | TTAGGGAATTACATAAAGGTTTTCTG |
| | GGTTTTCTTAAAAGAGCATGGGGGC | GGGCACATATTCAAGCTGAATGATAA |
| | CTCATCTTCAACAACCAATTAGGAA | AATTGAAGGTCACACAAAGCTAAGGT |
| | GCAGAAAAGTCCTCAGTGAGGAAGG | CTTTCAAATCCTGACCCAATTAGC |
| | AATAATGACATGTTGGAGCTAAGAT | (SEQ ID NO: 44) |
| | (SEQ ID NO: 43) | |
| hGJB2 GRE8 | GCCTGACACAGTCTGAGCCTCCTCA | CTGCCTTCCTGGCGTTTAGTGCGATT |
| | GGCGGCCTCAGGGGTTGGGATAGAG | TGTTTAGCCATGTGCTCCCTGGTGTG |
| | TGGAGAATTCAGGCAAGAATGCCAA | TGTTTTTGAATGTGTGTGAGATGGGT |
| | CCCTAGCTCCAGGCCTGGGACCCAC | TGTCTCTCGGGACCTGGCAGGTGCGG |
| | AGGCCTGGGGAAAAGAGTGGTTGCC | CCACCAGGTCAGGGCTGCCCCCCAAC |
| | CCGTCTTGAGACAGCCGAAAACTGT | CCTGTGCCTCCTTCCTCCTAGACTCT |
| | GTCCCCAGGATTGTTGGTTTCATAA | GGCCCCCTCAGTGCTGAGGGTGATAC |
| | AAGCAAGTAGCTAGGGAGGCCACAT | AGAGCACTTTTCAAGCTGGATTTGGA |
| | TTACAGGGGATCACAGAACACTTGG | ATGTGGCCTCTCCCCTCCAAACTCCT |
| | GTAGGGGCTTGCTGTAGGTGTCATC | GGAGATCATGCAAAGGCCTTTGGAGC |
| | AGGGAAGTGGGGGACGGCAGGAGGG | CAGCCAGTCACCTGGAAGGTGACATT |
| | ATGTGGCCCAGTACGCAGATGAAGA | CCCACCAGCTGAGGCCTCACCTTCAG |
| | CAGGTGATCATCCGCTGGGCCACAC | CGGGGGCTGGGCAGCTTTGGAGCCTG |
| | GTGGCAGGGATATGGGCAGAGTGAG | GGGCCAGCCAAGCTCACTCTGCCCAT |
| | CTTGGCTGGCCCCAGGCTCCAAAGC | ATCCCTGCCACGTGTGGCCCAGCGGA |
| | TGCCCAGCCCCCGCTGAAGGTGAGG | TGATCACCTGTCTTCATCTGCGTACT |
| | CCTCAGCTGGTGGGAATGTCACCTT | GGGCCACATCCCTCCTGCCGTCCCCC |
| | CCAGGTGACTGGCTGGCTCCAAAGG | ACTTCCCTGATGACACCTACAGCAAG |
| | CCTTTGCATGATCTCCAGGAGTTTG | CCCCTACCCAAGTGTTCTGTGATCCC |
| | GAGGGGAGAGGCCACATTCCAAATC | CTGTAAATGTGGCCTCCCTAGCTACT |
| | CAGCTTGAAAAGTGCTCTGTATCAC | TGCTTTTATGAAACCAACAATCCTGG |
| | CCTCAGCACTGAGGGGGCCAGAGTC | GGACACAGTTTTCGGCTGTCTCAAGA |
| | TAGGAGGAAGGAGGCACAGGGTTGG | CGGGGCAACCACTCTTTTCCCCAGGC |
| | GGGGCAGCCCTGACCTGGTGGCCGC | CTGTGGGTCCCAGGCCTGGAGCTAGG |
| | ACCTGCCAGGTCCCGAGAGACAACC | GTTGGCATTCTTGCCTGAATTCTCCA |
| | CATCTCACACACATTCAAAAACACA | CTCTATCCCAACCCCTGAGGCCGCCT |
| | CACCAGGGAGCACATGGCTAAACAA | GAGGAGGCTCAGACTGTGTCAGGC |
| | ATCGCACTAAACGCCAGGAAGGCAG | (SEQ ID NO: 60) |
| | (SEQ ID NO: 59) | |
| hGJB2 GRE9 | CGCCTCGGCCTCCCAAAGTGCTGGG | TTCTAGGTAGACAACTAAGATGTTCA |
| | ATTACAGGCGTGAGCCACCACCGTG | TCTTATGGTTTAATGTTTAGTTGTAA |
| | CCTGGCTTATACAAGTAATTGTAAA | AGGTTGTTTGCTTCTCATTTGGTTCC |
| | CGAAAAGGAAAAAATGGAGATACAG | AAGAAAGAGTATTTAGGCCAATTTCA |
| | TTTTCTCGTGCATCTTAAACTTTGG | GGGAGAAATATGTGTATAGATATATT |
| | TGCTTAAAAGCACCATTAAATTCTG | CATATGTCAAACTGATTAGTGCTGAA |
| | CTTTCACATGAACACACACAAGATT | TGTCACATTTCCATATTCTAATAACA |
| | ACCACGTTTGCTCTGGGCTGCTGCG | TTTCTAGCAAAGAAGAGGACACAGTG |
| | TATTGGAAGGACATACACATTCAAC | AAGAGAGAATTGCCCGCATTGTCATT |
| | AAATATTTGTTGAACTTCCATTCTG | GTCTCTTTCTGAGCCTAGAACGCCTA |
| | TACACAAAGCACAAAGAAAGATTCG | ACACTTGGGTGTGGAGAGACTCAGCC |
| | TTCACAGTCCGTGTGGGTACTGGAA | TCAATTCACTTTCTAGCAGCCACTGA |
| | AGCAGTTCCAGCCCTGCCTGCCAGG | GATGTGCTTGCCTGGGGTGCCCCCTG |
| | GGGCACCCCAGGCAAGCACATCTCA | GCAGGCAGGGCTGGAACTGCTTTCCA |
| | GTGGCTGCTAGAAAGTGAATTGAGG | GTACCCACACGGACTGTGAACGAATC |
| | CTGAGTCTCTCCACACCCAAGTGTT | TTTCTTTGTGCTTTGTGTACAGAATG |
| | AGGCGTTCTAGGCTCAGAAAGAGAC | GAAGTTCAACAAATATTTGTTGAATG |
| | AATGACAATGCGGGCAATTCTCTCT | TGTATGTCCTTCCAATACGCAGCAGC |
| | TCACTGTGTCCTCTTCTTTGCTAGA | CCAGAGCAAACGTGGTAATCTTGTGT |
| | AATGTTATTAGAATATGGAAATGTG | GTGTTCATGTGAAAGCAGAATTTAAT |
| | ACATTCAGCACTAATCAGTTTGACA | GGTGCTTTTAAGCACCAAAGTTTAAG |
| | TATGAATATATCTATACACATATTT | ATGCACGAGAAAACTGTATCTCCATT |
| | CTCCCTGAAATTGGCCTAAATACTC | TTTTCCTTTTCGTTTACAATTACTTG |
| | TTTCTTGGAACCAAATGAGAAGCAA | TATAAGCCAGGCACGGTGGTGGCTCA |
| | ACAACCTTTACAACTAAACATTAAA | CGCCTGTAATCCCAGCACTTTGGGAG |
| | CCATAAGATGAACATCTTAGTTGTC | GCCGAGGCGGGCGGATCACATGAGGT |
| | TACCTAGA | CGGGAG |
| | (SEQ ID NO: 45) | (SEQ ID NO: 46) |

In some embodiments, the GJB2 GRE is a non-human primate (e.g., Cynomolgus macaque) GJB2 enhancer. In some embodiments, the GJB2 GRE (e.g., a Cynomolgus macaque GJB2 enhancer) comprises nucleotide sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to any one of the GRE sequences as listed in Table 4.

TABLE 4
Cynomolgus macaque GJB2 (mfGJB2) Enhancer Sequences

| | Sense strand | Reverse Complement strand |
|---|---|---|
| mfGJB2 GRE1 | ATGGCACCAGCTTTTGAAAAAAGAA | CGATCACCCTCTATGTGCCATGGACT |
| | AACCTTTTTGCTGGTAGTCTGGCAA | TATCAGTGAGCAAGACAAAGCCCCTG |
| | GGAGACAGAAAAAAACCACTCACAT | CCCTCATTAATTCCCATCCTGTGTCC |
| | CTGCCTCCCCAGGCTGGGGGCTGGG | TCCTGTGCCATCCCCACCACCCCACC |
| | CCGGATTTTATAAGGATAGGGTAAT | TTGGTCAGGCCCAGTGTCTGGGTGTC |
| | GAGGGGTGGTCTGTTTGGATCTTGC | TTCAACTGCTCACCTGCCCGCTATTT |
| | AATGAGGTGCTGCTGGGAGGTGTGA | TGTTGCCCTGAAGTTCATCCAGACAT |
| | TCTGATTGGATCCTGCCATGGAGTG | CAGGGTGCCCTCTTGAAAATGCTAGT |
| | ATGCCAAAGCTCCATCTGATTGGAT | TACTATGACCTCTCTGCTCTAATCCC |
| | CCTGGATCCTGCCGTGTGTGCTCTG | CATGATGCGGTCCTGTCATCAATGGG |
| | CTTCTTAATGCAACCCCTGCTCCTC | ATAGAGCTGGTGTGACTGCACCAGAC |
| | AGTCTGAGCCCTTAGATTCTGCCCA | CAGTCAGGTTCAATTTTTATGAAAGG |
| | CGGTTGCACGCTTGGTTCACTTTGG | AAGTTGTGAGTTGCTTTTCAGTTGCC |
| | CATGCTCAGGTTACATGACCTTCAG | ATGGACCCCAAGCTGAAGGTCATGTA |
| | CTTGGGGTCCATGGCAACTGAAAAG | ACCTGAGCATGCCAAAGTGAACCAAG |
| | CAACTCACAACTTCCTTTCATAAAA | CGTGCAACCGTGGGCAGAATCTAAGG |
| | ATTGAACCTGACTGGTCTGGTGCAG | GCTCAGACTGAGGAGCAGGGGTTGCA |
| | TCACACCAGCTCTATCCCATTGATG | TTAAGAAGCAGAGCACACACGGCAGG |
| | ACAGGACCGCATCATGGGGATTAGA | ATCCAGGATCCAATCAGATGGAGCTT |
| | GCAGAGAGGTCATAGTAACTAGCAT | TGGCATCACTCCATGGCAGGATCCAA |
| | TTTCAAGAGGGCACCCTGATGTCTG | TCAGATCACACCTCCCAGCAGCACCT |
| | GATGAACTTCAGGGCAACAAAATAG | CATTGCAAGATCCAAACAGACCACCC |
| | CGGGCAGGTGAGCAGTTGAAGACAC | CTCATTACCCTATCCTTATAAAATCC |
| | CCAGACACTGGGCCTGACCAAGGTG | GGCCCAGCCCCCAGCCTGGGGAGGCA |
| | GGGTGGTGGGGATGGCACAGGAGGA | GATGTGAGTGGTTTTTTTCTGTCTCC |
| | CACAGGATGGGAATTAATGAGGGCA | TTGCCAGACTACCAGCAAAAAGGTTT |
| | GGGGCTTTGTCTTGCTCACTGATAA | TCTTTTTTCAAAAGCTGGTGCCAT |
| | GTCCATGGCACATAGAGGGTGATCG | (SEQ ID NO: 85) |
| | (SEQ ID NO: 84) | |
| mfGJB2 GRE2 | CCGTTAGGAAAAGAAAAACAGAAGG | TTGCTGGGTGCTATTTCTGTCCTTGT |
| | AATTGTGTTCTCTGGAGGGCAGGGC | CCATCTCCCCACCACAGCATGCACTT |
| | TCTGAGTACTGAGTCTCATGTTTTC | TGGATTCCAAGCATGCTCCTTGAGTG |
| | AAAGTCGGAAAGTGTCCACAGTTAA | TGACCCCGAGGCCCTCTGTGGGCTCT |
| | TATTTGGATGGGCCCACAGTGCCCG | TGGAGCAGGGCAGAGTTGGGTGTGCC |
| | TCTTGCTCGCCGGAGCCCAGGCCTG | AGGGCGCGACACGGGCCGGATGCCCT |
| | TCCCATCACAGACAAAGGGCTCTTG | GAGGTTGTTTGTTGTGCTGGGCTGGA |
| | CTGTGCACCTGTGGAGAGGGGAGCT | GGCGTTGGAAGAAATGTCCAAGGAGG |
| | TGGCTGGGGAAGGCAGGGTCAGCCT | CTGCTAGACTCAGTTCTTTCTTTCTG |
| | CTTTGTGCTCTTTTTGTTTGAAGCA | TTTCCCCTCCAGCTCCTCTGCTGGTA |
| | GAGTTTTGCAAAGGGAGTGGCTCTG | GAAGCTTCATGTCTCCCCGTCTCGTG |
| | GAAGAAAAGCAGAGCGTGGAGTGTC | AGCTGGCAAACACCCCGCTTCCGTGG |
| | AGAGGCCGGCGTGTTGTGAAATGCA | TTCAGTGTTGTCCTTGGCGGCGGGCG |
| | TAAGCCCTGGAGACCCTCTGTAACT | TGTGTGAAGGCCAGTTACAGAGGGTC |
| | GGCCTTCACACACGCCCGCCGCCAA | TCCAGGGCTTATGCATTTCACAACAC |
| | GGACAACACTGAACCACGGAAGCGG | GCCGGCCTCTGACACTCCACGCTCTG |
| | GGTGTTTGCCAGCTCACGAGACGGG | CTTTTCTTCCAGAGCCACTCCCTTTG |
| | GAGACATGAAGCTTCTACCAGCAGA | CAAAACTCTGCTTCAAACAAAAAGAG |
| | GGAGCTGGAGGGGAAACAGAAAGAA | CACAAAGAGGCTGACCCTGCCTTCCC |
| | AGAACTGAGTCTAGCAGCCTCCTTG | CAGCCAAGCTCCCCTCTCCACAGGTG |
| | GACATTTCTTCCAACGCCTCCAGCC | CACAGCAAGAGCCCTTTGTCTGTGAT |
| | CAGCACAACAAACAACCTCAGGGCA | GGGACAGGCCTGGGCTCCGGCGAGCA |
| | TCCGGCCCGTGTCGCGCCCTGGCAC | AGACGGGCACTGTGGGCCCATCCAAA |
| | ACCCAACTCTGCCCTGCTCCAAGAG | TATTAACTGTGGACACTTTCCGACTT |
| | CCCACAGAGGGCCTCGGGGTCACAC | TGAAAACATGAGACTCAGTACTCAGA |
| | TCAAGGAGCATGOTTGGAATCCAAA | GCCCTGCCCTCCAGAGAACACAATTC |
| | GTGCATGCTGTGGTGGGGAGATGGA | CTTCTGTTTTTCTTTTCCTAACGG |
| | CAAGGACAGAAATAGCACCCAGCAA | (SEQ ID NO: 87) |
| | (SEQ ID NO: 86) | |
| mfGJB2 GRE3 | AAAAAAGAATCACAATTGCCACCAA | CTGGGATGCCAATCTGAGGAATCCTT |
| | GGCTCTATGTTTTCGCAAAAGTCCA | CCTTTCCTAAGCAAAGGAGAAACAAA |
| | GCATTTAAAAGAAACTTCCTGCATG | ATAATTCTGATGGGGGAGTGACTGAC |
| | GCCTACATCTGCTGATTGGTAATTT | CCCAGTCTGGCTTACCGGCTGCTGTG |
| | GTCGTTCAGGTTAAAAACAAAACAA | AAGTCCTGAGTGTCCTCTGGCAGCCA |
| | GCGGGCATTGTTGTGATATCATCCT | CCTTTGAAAGCGCAGTGGTGTCCGGC |
| | TGATAACATCCCAAGAAAACTCTAG | ACTCGCCACTGAATAGCGTTTGTTCT |
| | AGCTGGCAAGAGAGGAAAGCAGATA | CAGAAGGGAGCCCAGTGGAAAATTTT |
| | ATGGTCAAAGCTGTCATCTGAGTTT | AAGCTGCAGTTAGGAGCCGTGTGTAT |
| | TAAAAACACTGTGATTTTTCTTTTA | GGCCTTGGAAACTGAAGATGTTCCTT |
| | AAGGAACATCTTCAGTTTCCAAGGC | TAAAAGAAAAATCACAGTGTTTTTAA |
| | CATACACACGGCTCCTAACTGCAGC | AACTCAGATGACAGCTTTGACCATTA |
| | TTAAAATTTTCCACTGGGCTCCCTT | TCTGCTTTCCTCTCTTGCCAGCTCTA |
| | CTGAGAACAAACGCTATTCAGTGGC | GAGTTTTCTTGGGATGTTATCAAGGA |
| | GAGTGCCGGACACCACTGCGCTTTC | TGATATCACAACAATGCCCGCTTGTT |
| | AAAGGTGGCTGCCAGAGGACACTCA | TTGTTTTTAACCTGAACGACAAATTA |
| | GGACTTCACAGCAGCCGGTAAGCCA | CCAATCAGCAGATGTAGGCCATGCAG |
| | GACTGGGGTCAGTCACTCCCCCATC | GAAGTTTCTTTTAAATGCTGGACTTT |
| | AGAATTATTTTGTTTCTCCTTTGCT | TGCGAAAACATAGAGCCTTGGTGGCA |
| | TAGGAAAGGAAGGATTCCTCAGATT | ATTGTGATTCTTTTTT |
| | GGCATCCCAG | (SEQ ID NO: 89) |
| | (SEQ ID NO: 88) | |
| mfGJB2GRE4 | ATAATGAGCAACATAAGGTTAAAAT | GCCATTCTGCATTAGGTTTGGTTAAA |
| | AACATTGCAACCCCATGGAAGCAAG | AAAATGAAACTATCGGCTGAGCTGGG |
| | AGAAATGGAAATTATTAATAAATGG | TAAACACTGGTTTTGGTCAAATATGG |
| | ACCACATGTAAGGGAATGCTGTGGT | AATGAGACGGTGCCACGTATTTCCAA |
| | TCTATTGTAGAGATTACAGAGAGCA | TGGGGCTGCTCAGTGACTCGTGAGCG |
| | ATTTAGGAGAGCCAGGCGCTGGGGG | TGTGTGGAATGTGAGTCTGGTCTCCC |
| | CAAGAGGGAAATGAAACGAAAACCG | AGGATACTTCAAAGGTGTACAGGTCC |
| | AAGGGATTTGTTCAGGAAGAAAAAT | CTTTGTCGGTGCCACACGTCCCCGCT |
| | GAAAACAGATAAAAGGTGTTCATTT | CATGGGTATAACATGCCTGGAGATTT |
| | CAAAGCTTCCCTCTTTCCCAGCATT | GCACAGGCAGTTTTCAGGGCTGTCAA |
| | TTTCTGAAGTAGAGTTTGAAAGGAA | GGAGCCAGGTGACCCAGAACGGGAGG |
| | AGCAAAATAACTGCAAACCAATACA | CGGGGCTGGAGATCTCTGGACGTCGT |
| | GTGGCACGAGTTCACTGACGCAGAG | TCCTAGCTCTGCGTCAGTGAACTCGT |
| | CTAGGAACGACGTCCAGAGATCTCC | GCCACTGTATTGGTTTGCAGTTATTT |
| | AGCCCCGCCTCCCGTTCTGGGTCAC | TGCTTTCCTTTCAAACTCTACTTCAG |
| | CTGGCTCCTTGACAGCCCTGAAAAC | AAAAATGCTGGGAAAGAGGGAAGCTT |
| | TGCCTGTGCAAATCTCCAGGCATGT | TGAAATGAACACCTTTTATCTGTTTT |
| | TATACCCATGAGCGGGGACGTGTGG | CATTTTTCTTCCTGAACAAATCCCTT |
| | CACCGACAAAGGGACCTGTACACCT | CGGTTTTCGTTTCATTTCCCTCTTGC |
| | TTGAAGTATCCTGGGAGACCAGACT | CCCCAGCGCCTGGCTCTCCTAAATTG |
| | CACATTCCACACACGCTCACGAGTC | CTCTCTGTAATCTCTACAATAGAACC |
| | ACTGAGCAGCCCCATTGGAAATACG | ACAGCATTCCCTTACATGTGGTCCAT |
| | TGGCACCGTCTCATTCCATATTTGA | TTATTAATAATTTCCATTTCTCTTGC |
| | CCAAAACCAGTGTTTACCCAGCTCA | TTCCATGGGGTTGCAATGTTATTTTA |
| | GCCGATAGTTTCATTTTTTTAACCA | ACCTTATGTTGCTCATTAT |
| | AACCTAATGCAGAATGGC | (SEQ ID NO: 91) |
| | (SEQ ID NO: 90) | |
| mfGJB2 GRE5 | CACGTCTTGTAATTTTTTTACTGAA | ACCTTTAAGAAAAATCTGCCAAAAGA |
| | TGTTAGACATTGCATATAAAAGACT | TTTGGAGCTGGATTGGAATTTAGAAG |
| | ATCCAGGAGTGTTTTGTTTTTGTTT | TCCACCAAATGCAAAAATAGTTTGGC |
| | TTTCTAGTGAGTGCAAGTCCCTTGC | TCAACGTCACCCCCATCCGTGATTTT |
| | TCTCTGCCAGTTGGCTGGAATGAGA | ACTGCAAAAGTGGCTGTGGAGGCAAG |
| | ATCTGATCAGATTTCATCAAGAGTC | ACTGGAAAACAGTTAAACAATTTCAT |
| | AGGTTGAGCTGAGACTGAGCGGTAG | AGTGCTTGAATTGTGGAGCCATGTTA |
| | TGTTCACTAAATTGAGTGCACCACT | GATGCAAGGGAAGCCAAAATGATATG |
| | GATATCTAATGGAAACAAGGACATT | AAATCTATGTCTCAACCTGCTTCCAG |
| | TTACTTTGCTCCTCAGCCTAACCTG | CTCACACATTAGGAGAACCAAAGAAA |
| | AATTTCCTATGCCACCACTGTATAA | CCAGCCATTATACAGTGGTGGCATAG |
| | TGGCTGGTTTCTTTGGTTCTCCTAA | GAAATTCAGGTTAGGCTGAGGAGCAA |
| | TGTGTGAGCTGGAAGCAGGTTGAGA | AGTAAAATGTCCTTGTTTCCATTAGA |
| | CATAGATTTCATATCATTTTGGCTT | TATCAGTGGTGCACTCAATTTAGTGA |
| | CCCTTGCATCTAACATGGCTCCACA | ACACTACCGCTCAGTCTCAGCTCAAC |
| | ATTCAAGCACTATGAAATTGTTTAA | CTGACTCTTGATGAAATCTGATCAGA |
| | CTGTTTTCCAGTCTTGCCTCCACAG | TTCTCATTCCAGCCAACTGGCAGAGA |
| | CCACTTTTGCAGTAAAATCACGGAT | GCAAGGGACTTGCACTCACTAGAAAA |
| | GGGGGTGACGTTGAGCCAAACTATT | AACAAAAACAAAACACTCCTGGATAG |
| | TTTGCATTTGGTGGACTTCTAAATT | TCTTTTATATGCAATGTCTAACATTC |
| | CCAATCCAGCTCCAAATCTTTTGGC | AGTAAAAAAATTACAAGACGTG |
| | AGATTTTTCTTAAAGGT | (SEQ ID NO: 93) |
| | (SEQ ID NO: 92) | |
| mfGJB2 GRE6 | CGGCAGAGACCTACAGACCAAAGTA | TTGCAAAGCCTCTCTTTGGGGATCGG |
| | CATTTCACACTGGATCCAGGACACA | ACATGGAGTCTGTGGTCTTAGAATTC |
| | CATCAGTCTGAAAGCACACACATGA | AGAACTGGGATAATGTGTTAGCCACT |
| | ACCAAACGTTTCCTAAAGCATTACT | CATCTAAGCCATTCCTTAAACGCTTT |
| | TACCCTTGCTAATAGCAACACATTC | CAGAGCCATCTCCACTGTGGGGAAAG |
| | TCATATTCTTTTATACTTCATTTAA | AAGTTCTTTGTGTTCTCTCATTTAGT |
| | TTTCATTTAAAAAAGAAAAAGATAG | CTCATTCTAAAAAAAAAAAAAAAAAA |
| | GAAAGAAATCTATTTCTCCGCCCAT | AAAAAAGGCTATTGCAGTACCCAGAG |
| | TAATAAGGTCAGACGCAGCAACGCT | CGCACAGTAGATGGCACTGACACTTG |
| | AGACTAGAAGAAAAGTTTACCTACT | TCGGAAAGCTGTGCGCACTCAGGAGG |
| | GATTTTTCTCCCACCTCCTGAGTGC | TGGGAGAAAAATCAGTAGGTAAACTT |
| | GCACAGCTTTCCGACAAGTGTCAGT | TTCTTCTAGTCTAGCGTTGCTGCGTC |
| | GCCATCTACTGTGCGCTCTGGGTAC | TGACCTTATTAATGGGCGGAGAAATA |
| | TGCAATAGCCTTTTTTTTTTTTTTT | GATTTCTTTCCTATCTTTTTCTTTTT |
| | TTTTTTTTTAGAATGAGACTAAATG | TAAATGAAATTAAATGAAGTATAAAA |
| | AGAGAACACAAAGAACTTCTTTCCC | GAATATGAGAATGTGTTGCTATTAGC |
| | CACAGTGGAGATGGCTCTGAAAGCG | AAGGGTAAGTAATGCTTTAGGAAACG |
| | TTTAAGGAATGGCTTAGATGAGTGG | TTTGGTTCATGTGTGTGCTTTCAGAC |
| | CTAACACATTATCCCAGTTCTGAAT | TGATGTGTGTCCTGGATCCAGTGTGA |
| | TCTAAGACCACAGACTCCATGTCCG | AATGTACTTTGGTCTGTAGGTCTCTG |
| | ATCCCCAAAGAGAGGCTTTGCAA | CCG |
| | (SEQ ID NO: 94) | (SEQ ID NO: 95) |
| mfGJB2 GRE7 | GGTGTGTATATCAGGTGGTTACTTT | TGTATGCTTGTGTGAATTTTTGTTTT |
| | ACAAAACAGGATGTGGCAAGCTGGA | TAACTTTTTGTGAGTGCCCTAACAAA |
| | CCTGATAGACACATCAAAGCCTCTG | GACTACACATTGGGAATACAAACACC |
| | AACAGAGTTCAGGGCATGAAATGGT | AGAGCAATGGAAACAGTGACACTTTT |
| | TTCTTTTGGGGGTCTTCAGGAACAA | GTGGAAGGTCCACGTGGCCGTTCAGG |
| | TTTCATGAAAGCTAAATCATGAAAG | TGGTTGTAACACAGGCTGGCGCCCCT |
| | ATAGCAGACTTTTGCCAGGAAAAAA | GCCCTGCAGTGGGAATCCCCAAGGCA |
| | AAACAAAACAAAACGAGACTAGTGA | TTGGGGGATTCAGCCTCTCGCAGTGA |
| | TTAGTTTGGCGTTTTCGGTTTCTTT | CCTCTTGTAAGACAGCAGATGGCAGC |
| | GAGAAGCGAAATAACTTATCAAGGA | AGAGAGAGGCTTTGCACATCCCTGCA |
| | CTCTTTGTGCCGCTTGATGTTCTAA | GGTTCTAGTTTGCAGAAAGGGCTTCT |
| | TCGGTTGATGGGTCTCTCAGAAGCC | GAGAGACCCATCAACCGATTAGAACA |
| | CTTTCTGCAAACTAGAACCTGCAGG | TCAAGCGGCACAAAGAGTCCTTGATA |
| | GATGTGCAAAGCCTCTCTCTGCTGC | AGTTATTTCGCTTCTCAAAGAAACCG |
| | CATCTGCTGTCTTACAAGAGGTCAC | AAAACGCCAAACTAATCACTAGTCTC |
| | TGCGAGAGGCTGAATCCCCCAATGC | GTTTTGTTTTGTTTTTTTTTCCTGGC |
| | CTTGGGGATTCCCACTGCAGGGCAG | AAAAGTCTGCTATCTTTCATGATTTA |
| | GGGCGCCAGCCTGTGTTACAACCAC | GCTTTCATGAAATTGTTCCTGAAGAC |
| | CTGAACGGCCACGTGGACCTTCCAC | CCCCAAAAGAAACCATTTCATGCCCT |
| | AAAAGTGTCACTGTTTCCATTGCTC | GAACTCTGTTCAGAGGCTTTGATGTG |
| | TGGTGTTTGTATTCCCAATGTGTAG | TCTATCAGGTCCAGCTTGCCACATCC |
| | TCTTTGTTAGGGCACTCACAAAAAG | TGTTTTGTAAAGTAACCACCTGATAT |
| | TTAAAAACAAAAATTCACACAAGCA | ACACACC |
| | TACA | (SEQ ID NO: 97) |
| | (SEQ ID NO: 96) | |
| mfGJB2 GRE8 | GGTCAGGATTTGAAAGACCTTAGCT | CACCATCATCTTAGCTCCAACATGTC |
| | TTGTGTGACCTTCAGTTTTATCATT | ATTATTCCTTCCTCACTGAGGACTTT |
| | CAGTTTGAATATGTGCCCCAGAAAA | TCTGCCTCCTAATTGGTTGTTGAAGA |
| | CCTTTATGTAATTTCCTAATATTTC | CGAGGCCCCCATGCTCTTTTAAGAAA |
| | AGTAACATATTTCACAACATACAAG | ACCTGTTCTGCCCCAGGCTTGGCTGC |
| | CAGCACATTCTCTTTTTTTAGAATG | GACGGGTACTGACTCATAGAGAAGTA |
| | GTGTCTCGCTGATGACTTTGACGAC | GAAAGGCCTGCTGAATCATCAACACT |
| | AGCTCACGTGAGAGGGAAGTATTTC | CCCGCGACGCCCCTGCATTTTCATTA |
| | AGCAATCAGACCGAAGGAGAATCCA | ATGACGGCCTCCCTGCTACACGTGAA |
| | AAAACCCCACTATTGCGGGGTCAAG | TCACTCCAGCCTGAGATCTGAAACCC |
| | AGTGCACGTGTTTGAATTCTGAAAG | GGGCACACCCCAGGGGCGAGGTGACA |
| | ATGTAAGCCAAGGCAAACAGAAGGA | CTGAGTGAGCCCAGCTGTGTCCCCTT |
| | AATGATCTTCCACTAATCCCTGCAT | CAGGAGAAGTCAGAGCACAGGGCTCT |
| | TTACTTCCTCCTCTCTGGAGGGGAC | GTGTGTGTGGCCGTCCCCTCCAGAGA |
| | GGCCACACACACAGAGCCCTGTGCT | GGAGGAAGTAAATGCAGGGATTAGTG |
| | CTGACTTCTCCTGAAGGGGACACAG | GAAGATCATTTCCTTCTGTTTGCCTT |
| | CTGGGCTCACTCAGTGTCACCTCGC | GGCTTACATCTTTCAGAATTCAAACA |
| | CCCTGGGGTGTGCCCGGGTTTCAGA | CGTGCACTCTTGACCCCGCAATAGTG |
| | TCTCAGGCTGGAGTGATTCACGTGT | GGGTTTTTGGATTCTCCTTCGGTCTG |
| | AGCAGGGAGGCCGTCATTAATGAAA | ATTGCTGAAATACTTCCCTCTCACGT |
| | ATGCAGGGGCGTCGCGGGAGTGTTG | GAGCTGTCGTCAAAGTCATCAGCGAG |
| | ATGATTCAGCAGGCCTTTCTACTTC | ACACCATTCTAAAAAAAGAGAATGTG |
| | TCTATGAGTCAGTACCCGTCGCAGC | CTGCTTGTATGTTGTGAAATATGTTA |
| | CAAGCCTGGGGCAGAACAGGTTTTC | CTGAAATATTAGGAAATTACATAAAG |
| | TTAAAAGAGCATGGGGGCCTCGTCT | GTTTTCTGGGGCACATATTCAAACTG |
| | TCAACAACCAATTAGGAGGCAGAAA | AATGATAAAACTGAAGGTCACACAAA |
| | AGTCCTCAGTGAGGAAGGAATAATG | GCTAAGGTCTTTCAAATCCTGACC |
| | ACATGTTGGAGCTAAGATGATGGTG | (SEQ ID NO: 99) |
| | (SEQ ID NO: 98) | |
| mfGJB2 GRE9 | GTTTTTTCATGCATCTTAAACTTTG | GTTTCCTTCTCATTTGGTTTCAAGGA |
| | GTGCTTAAAGAAAAGCACCATTAAA | AGACAGTGTTTAGGACAATTTCAGGG |
| | TCCTGCTCTCACACGAACACACACA | AGAAATATGTGTCTATGTAGATATAC |
| | AGATTACCACGTTTGCTCTGGGCTG | TCATATGTCAAACTGATTAGTGCTGA |
| | CCGCGTATAGGAAGGACATATACAT | ATTCTCAATCGACGGGTCACATTTCC |
| | TCAATAAATATTTGTTGAACTTCCA | ACATTCTAATAACATTTCTAGCAAAG |
| | TTCTGTACACAAAGCACAAAGAAAG | AAAGGGACACAGTGAAGAGAGAATTG |
| | ATTCGTTCACAGTCCGCGTGGGTAC | CCCGCATTGTCATTGTCTCTCTTTGA |
| | AGGAAAGCAGTTCCAGCCCTGCCTG | GCCTAGAACACCTAACACTTGGGTAT |
| | CCAGGGGGCACCCCAGGCAAGCACA | AGAGAGAGACTCAGCCTCAACTCGCT |
| | TCTCAGTGGCTGCAAGAAAGTCAGC | GACTTTCTTGCAGCCACTGAGATGTG |
| | GAGTTGAGGCTGAGTCTCTCTCTAT | CTTGCCTGGGGTGCCCCCTGGCAGGC |
| | ACCCAAGTGTTAGGTGTTCTAGGCT | AGGGCTGGAACTGCTTTCCTGTACCC |
| | CAAAGAGAGACAATGACAATGCGGG | ACGCGGACTGTGAACGAATCTTTCTT |
| | CAATTCTCTCTTCACTGTGTCCCTT | TGTGCTTTGTGTACAGAATGGAAGTT |
| | TCTTTGCTAGAAATGTTATTAGAAT | CAACAAATATTTATTGAATGTATATG |
| | GTGGAAATGTGACCCGTCGATTGAG | TCCTTCCTATACGCGGCAGCCCAGAG |
| | AATTCAGCACTAATCAGTTTGACAT | CAAACGTGGTAATCTTGTGTGTGTTC |
| | ATGAGTATATCTACATAGACACATA | GTGTGAGAGCAGGATTTAATGGTGCT |
| | TTTCTCCCTGAAATTGTCCTAAACA | TTTCTTTAAGCACCAAAGTTTAAGAT |
| | CTGTCTTCCTTGAAACCAAATGAGA | GCATGAAAAAAC |
| | AGGAAAC | (SEQ ID NO: 101) |
| | (SEQ ID NO: 100) | |

In some embodiments, the human GJB2 GREs share homology with the mfGJB2 GREs. In some embodiments, the human GJB2 GREs correspond to mfGJB2 GREs as set forth in Table 5:

TABLE 5
Homology between Human GJB2 GREs and mfGJB2 GREs

| | |
|---|---|
| hGJB2 GRE9 | mfGJB2 GRE9 |
| hGJB2 GRE7 | mfGJB2 GRE8 |
| hGJB2 GRE5 | mfGJB2 GRE7 |
| hGJB2 GRE3 | mfGJB2 GRE6 |
| hGJB2 GRE2 | mfGJB2 GRE5 |

In some embodiments, the isolated nucleic acid comprises one or more (e.g., 1, 2, 3, 4, 5, 6, 7, 9, or more) enhancers (e.g., GJB2 enhancers). In some embodiments, the isolated nucleic acid comprises more than one enhancer, and the more than one enhancer are the same enhancers or different enhancers. In some embodiments, the GJB2 GRE is positioned 5′ to the promoter. In other embodiments, the GJB2 GRE is positioned 3′ to the promoter. In some embodiments, the presence of the GJB2 enhancer(s) in the isolated nucleic acid facilitates cell-type specific expression of the GJB2 protein encoded by the isolated nucleic acid. In some embodiments, cells that normally express the GJB2 gene (e.g., fibrocytes and supporting cells of the organ of Corti and nearby regions) have the transcriptional network to activate GJB2 expression regulated by the GJB2 enhancers, but not in cells that do not normally express GJB2 (e.g., hair cells and spiral ganglion neurons).

In some embodiments, the expression cassette of the isolated nucleic acid further comprises a 5′ UTR. In some embodiments, the 5′ UTR is a native 5′ UTR of the genomic GJB2 gene. The 5′ untranslated region (5′ UTR) (also known as a leader sequence or leader RNA) is the region of an mRNA that is directly upstream of the initiation codon. The 5′ UTR plays important roles in both transcriptional and translational regulation of the downstream gene (e.g., the GJB2 gene). In some embodiments, the isolated nucleic acid comprising a nucleotide sequence encoding a GJB2 5′ UTR is also capable of expression GJB2 in a cell-specific manner (e.g., expressing GJB2 in cells that normally express it). In some embodiments, the nucleotide sequence encoding the GJB2 5′ UTR comprises a portion of a nucleotide sequence encoding a full-length human GJB2 gene 5′ UTR. In some embodiments, the 5′ UTR is a human GJB2 gene exon 1 5′ UTR. In some embodiments, the nucleotide sequence encoding a 5′ UTR comprises at least 100 consecutive nucleotides, at least 200 consecutive nucleotides, at least 300 consecutive nucleotides, at least 400 consecutive nucleotides, at least 500 consecutive nucleotides, at least 600 consecutive nucleotides, at least 700 consecutive nucleotides, at least 800 consecutive nucleotides, at least 900 consecutive nucleotides, at least 1000 consecutive nucleotides, or more of a native full-length 5′ UTR (e.g., the human GJB2 gene exon 1 5′ UTR). In some embodiments, the expression cassette comprises a nucleotide sequence encoding the 5′ UTR having at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to a nucleotide sequence encoding a human GJB2 gene 5′ UTR (e.g., human GJB2 exon 1 5′ UTR). In some embodiments, the expression cassette comprises a nucleotide sequence encoding the 5′ UTR having at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to a nucleotide sequence encoding a consecutive 300 bp of a human GJB2 gene 5′ UTR (e.g., the human GJB2 gene exon 1 5′ UTR) as set forth in SEQ ID NO: 53. In some embodiments, an exemplary nucleotide sequence encoding the 300 bp of the human GJB2 gene exon 1 5′ UTR has a nucleotide sequence as set forth in SEQ ID NO: 53:

GGGGTGCGGTTAAAAGGCGCCACGGCGGGAGACAGGTGTTGCGGCCCCGC
AGCGCCCGCGCGCTCCTCTCCCCGACTCGGAGCCCCTCGGCGGCGCCCGG
CCCAGGACCCGCCTAGGAGCGCAGGAGCCCCAGCGCAGAGACCCCAACGC
CGAGACCCCCGCCCCGGCCCCGCCGCGCTTCCTCCCGACGCAGAGCAAAC
CGCCCAGAGTAG

In some embodiments, the cell specific GJB2 expression is achieved by incorporation of a nucleotide sequence encoding a basal promoter and a GJB2 5′ UTR or a portion thereof (basal promoter/5′ UTR). In some embodiments, an expression cassette (e.g., GJB2 expression cassette) comprises a nucleotide sequence encoding a 5′ UTR. In some embodiments, the isolated nucleic acid can further comprise additional nucleotide sequence encoding one or more GJB2 GREs (e.g., GJB2 enhancers). The nucleotide sequence encoding the GJB2 GREs and the nucleotide sequence encoding the basal promoter/5′ UTR can be placed in any order. In some embodiments, the nucleotide sequence encoding the GJB2 GREs is placed 5′ to the nucleotide sequence encoding the basal promoter/5′ UTR. In some embodiments, the isolated nucleic acid comprising a nucleotide sequence encoding a GJB2 basal promoter/5′ UTR is also capable of expressing GJB2 in a cell-specific manner (e.g., expressing GJB2 in cells that normally express it). In some embodiments, the nucleotide sequence encoding the basal promoter/5′ UTR comprises a portion of a nucleotide sequence encoding a full-length human GJB2 gene 5′ UTR. In some embodiments, the 5′ UTR comprises at least 100 consecutive nucleotides, at least 200 consecutive nucleotides, at least 300 consecutive nucleotides, at least 400 consecutive nucleotides, at least 500 consecutive nucleotides, at least 600 consecutive nucleotides, at least 700 consecutive nucleotides, at least 800 consecutive nucleotides, at least 900 consecutive nucleotides, at least 1000 consecutive nucleotides, or more of a native full-length 5′ UTR (e.g., the GJB2 5′ UTR). In some embodiments, the 5′ UTR is a human GJB2 gene exon 1 5′ UTR. In some embodiments, the expression cassette comprises a nucleotide sequence encoding a basal promoter/5′ UTR having at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to a nucleotide sequence encoding the basal promoter and about 300 bp of a human GJB2 gene 5′ UTR (e.g., the human GJB2 gene exon 1 5′ UTR) (SEQ ID NO: 30). In some embodiments, an exemplary nucleotide sequence encoding the 300 bp of the human GJB2 gene basal promoter/exon 1 5′ UTR has a nucleotide sequence as set forth in SEQ ID NO: 30:

GGGTTCGGGGCTTTCGGGGGCGGTCGGGGGTTCGCGGACCCGGGAAGCTC
TGAGGACCCAGAGGCCGGGCGCGCTCCGCCCGCGGCGCCGCCCCCTCCGT
AACTTTCCCAGTCTCCGAGGGAAGAGGCGGGGTGTGGGGTGCGGTTAAAA
GGCGCCACGGCGGGAGACAGGTGTTGCGGCCCCGCAGCGCCCGCGCGCTC
CTCTCCCCGACTCGGAGCCCCTCGGCGGCGCCCGGCCCAGGACCCGCCTA
GGAGCGCAGGAGCCCCAGCGCAGAGACCCCAACGCCGAGACCCCCGCCCC
GGCCCCGCCGCGCTTCCTCCCGACGCAGAGCAAACCGCCCAGAGTAG

In some embodiments, a nucleotide sequence encoding a basal promoter/5′ UTR (e.g., a human GJB2 basal promoter/exon 1 5′ UTR) within the expression cassette (e.g., GJB2 expression cassette) further comprises an intron or a portion thereof. In some embodiments, the expression cassette of the isolated nucleic acid (e.g., GJB2 expression cassette) further comprises a conserved sequence of intron 1 of the GJB2 gene. In some embodiments, the nucleotide sequence encoding an intron (e.g., human GJB2 intron 1) has at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 54. An exemplary nucleotide sequence encoding the conserved sequence of GJB2 intron 1 is set forth in SEQ ID NO: 54:

AAGCAGGTGAGTTTGTGGTGTCGCCGATGTCCCTTCGGGGTACTCTAGCG
CAGCCGCCTGGCTACTTGACCCACTGCCACCAAACGTTTTAAATTCACCG
AAAGCTTAGCTTCGAAGCAAAGCTCCGTTTCGCCGGTGAAGCAGGAAGCC
TTCGCTGCAGGAACTGACCTTTACCTCTTGGAGCGGCTTCTGCAGAAAAA
TCCCCGGGCAGAGATTTGGGCGGAGTTTGCCTAGAACTAACGCGGAGCCA
GCCGATCCCGGCCTACCCCGGGGCCAAGATTTTAAGGGGTGAAGAGTCCC
TTTTGCCTTTTCTGGATCCTGGTGATTCACCTAGTGTCTTCCCTAAGGAA
CTGAACCAACTCCTCCGCTGGCCTCTGGCAGCCCTCCAGGCGGTGCAGGA
TGGCGTGGGCCCGGTAGGAAGCTGCATGTAACCGCCCAGGGTCGGGAGGC
CAGGAGGGCAGCTCCTCCTCTGACTTGAATATTGAAAACAAGAGGATGCT
TTTAAGAAAAAGAAGAAGGAGGATTCACTACCAGCTCTGAAGGGTGGAAA
AGAGATGATTCATCCGGATTGTGGAGAGGGTGGAATCTTGTTTAGGAGAG
CGTTGGTTGTGGCAGGCAGGGTGTAACTATGAATCAGTGAAGACAATTCA
CATCCTGGGATGAAAAGAAGGCCATGGGCTCACAGGAGATTATCCACTGG
CCTCTCCACATCCGCTTGCAGTAAGGAGTGTGGGACTCTCCCAAGCTTCA
GCGCTGAACTGCAATGCAGTGACGTCGCTTAAGA

In some embodiments, the nucleotide sequence encoding a basal promoter/5′ UTR/intron has at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 31. An exemplary nucleotide sequence encoding human GJB2 basal promoter/5′UTR/conserved sequence of intron 1 is set forth in SEQ ID NO: 31:

GGGTTCGGGGCTTTCGGGGGCGGTCGGGGGTTCGCGGACCCGGGAAGCTC
TGAGGACCCAGAGGCCGGGCGCGCTCCGCCCGCGGCGCCGCCCCCTCCGT
AACTTTCCCAGTCTCCGAGGGAAGAGGCGGGGTGTGGGGTGCGGTTAAAA
GGCGCCACGGCGGGAGACAGGTGTTGCGGCCCCGCAGCGCCCGCGCGCTC
CTCTCCCCGACTCGGAGCCCCTCGGCGGCGCCCGGCCCAGGACCCGCCTA
GGAGCGCAGGAGCCCCAGCGCAGAGACCCCAACGCCGAGACCCCCGCCCC
GGCCCCGCCGCGCTTCCTCCCGACGCAGAGCAAACCGCCCAGAGTAGAAG
CAGGTGAGTTTGTGGTGTCGCCGATGTCCCTTCGGGGTACTCTAGCGCAG
CCGCCTGGCTACTTGACCCACTGCCACCAAACGTTTTAAATTCACCGAAA
GCTTAGCTTCGAAGCAAAGCTCCGTTTCGCCGGTGAAGCAGGAAGCCTTC
GCTGCAGGAACTGACCTTTACCTCTTGGAGCGGCTTCTGCAGAAAAATCC
CCGGGCAGAGATTTGGGCGGAGTTTGCCTAGAACTAACGCGGAGCCAGCC
GATCCCGGCCTACCCCGGGGCCAAGATTTTAAGGGGTGAAGAGTCCCTTT
TGCCTTTTCTGGATCCTGGTGATTCACCTAGTGTCTTCCCTAAGGAACTG
AACCAACTCCTCCGCTGGCCTCTGGCAGCCCTCCAGGCGGTGCAGGATGG
CGTGGGCCCGGTAGGAAGCTGCATGTAACCGCCCAGGGTCGGGAGGCCAG
GAGGGCAGCTCCTCCTCTGACTTGAATATTGAAAACAAGAGGATGCTTTT
AAGAAAAAGAAGAAGGAGGATTCACTACCAGCTCTGAAGGGTGGAAAAGA
GATGATTCATCCGGATTGTGGAGAGGGTGGAATCTTGTTTAGGAGAGCGT
TGGTTGTGGCAGGCAGGGTGTAACTATGAATCAGTGAAGACAATTCACAT
CCTGGGATGAAAAGAAGGCCATGGGCTCACAGGAGATTATCCACTGGCCT
CTCCACATCCGCTTGCAGTAAGGAGTGTGGGACTCTCCCAAGCTTCAGCG
CTGAACTGCAATGCAGTGACGTCGCTTAAGA

In some embodiments, the expression cassette (e.g., GJB2 expression cassette) comprises a nucleotide sequence encoding a proximal promoter of the human GJB2 gene. In some embodiments, the proximal promoter of the human GJB2 gene has at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 102. In some embodiments, an exemplary nucleotide sequence encoding the human GJB2 gene proximal promoter has a nucleotide sequence as set forth in SEQ ID NO: 102. In some embodiments, the expression cassette (e.g., GJB2 expression cassette) comprises SEQ ID NO: 102:

GACCTCGAAGGGGACTTGGGGGGTTCGGGGCTTTCGGGGGCGGTCGGGGG
TTCGCGGACCCGGGAAGCTCTGAGGACCCAGAGGCCGGGCGCGCTCCGCC
CGCGGCGCCGCCCCCTCCGT

In some embodiments, the expression cassette (e.g., GJB2 expression cassette) comprises a nucleotide sequence encoding a 5′ UTR of the human GJB2 gene. In some embodiments, the 5′ UTR of the human GJB2 gene has at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 103 or CC. In some embodiments, an exemplary nucleotide sequence encoding the human GJB2 gene 5′ UTR has a nucleotide sequence as set forth in SEQ ID NO: 103 or CC. In some embodiments, an exemplary nucleotide sequence encoding the human GJB2 gene 5′ UTR has a nucleotide sequence comprising SEQ ID NO: 103 and SEQ ID NO: 104. In some embodiments, the expression cassette (e.g., GJB2 expression cassette) comprises SEQ ID NO: 103:

AACTTTCCCAGTCTCCGAGGGAAGAGGCGGGGTGTGGGGTGCGGTTAAAA
GGCGCCACGGCGGGAGACAGGTGTTGCGGCCCCGCAGCGCCCGCGCGCTC
CTCTCCCCGACTCGGAGCCCCTCGGCGGCGCCCGGCCCAGGACCCGCCTA
GGAGCGCAGGAGCCCCAGCGCAGAGACCCCAACGCCGAGACCCCCGCCCC
GGCCCCGCCGCGCTTCCTCCCGACGCAG

In some embodiments, the expression cassette (e.g., GJB2 expression cassette) comprises SEQ ID NO: 104:

AGCAAACCGCCCAGAGTAGAAG

In some embodiments, the expression cassette (e.g., GJB2 expression cassette) comprises a nucleotide sequence encoding a proximal promoter and a 5′ UTR of the human GJB2 gene. In some embodiments, the proximal promoter and the 5′ UTR of the human GJB2 gene has at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 105. In some embodiments, an exemplary nucleotide sequence encoding the human GJB2 gene proximal promoter and 5′ UTR has a nucleotide sequence as set forth in SEQ ID NO: 105. In some embodiments, the expression cassette (e.g., GJB2 expression cassette) comprises SEQ ID NO: 105:

GACCTCGAAGGGGACTTGGGGGGTTCGGGGCTTTCGGGGGCGGTCGGGGG
TTCGCGGACCCGGGAAGCTCTGAGGACCCAGAGGCCGGGCGCGCTCCGCC
CGCGGCGCCGCCCCCTCCGTAACTTTCCCAGTCTCCGAGGGAAGAGGCGG
GGTGTGGGGTGCGGTTAAAAGGCGCCACGGCGGGAGACAGGTGTTGCGGC
CCCGCAGCGCCCGCGCGCTCCTCTCCCCGACTCGGAGCCCCTCGGCGGCG
CCCGGCCCAGGACCCGCCTAGGAGCGCAGGAGCCCCAGCGCAGAGACCCC
AACGCCGAGACCCCCGCCCCGGCCCCGCCGCGCTTCCTCCCGACGCAGAG
CAAACCGCCCAGAGTAGAAG

An isolated nucleic acid described herein may also contain an artificial intron, desirably located between the promoter/enhancer sequence and the protein coding sequence (e.g., nucleotide sequence encoding GJB2 protein). In some embodiments, an intron is a synthetic or artificial (e.g., heterologous) intron. Examples of synthetic introns include an intron sequence derived from SV-40 (referred to as the SV-40 T intron sequence) and intron sequences derived from chicken beta-actin gene. In some embodiments, a transgene described by the disclosure comprises one or more (1, 2, 3, 4, 5, or more) artificial introns. In some embodiments, the one or more artificial introns are positioned between a promoter and a nucleotide sequence encoding the GJB2 protein.

In some embodiments, the expression cassette (e.g., the GJB2) further comprises a nucleotide sequence encoding a 3′ UTR located 3′ of the nucleotide sequence encoding the GJB2 protein. In some embodiments, the 3′ UTR is a GJB2 gene 3′ UTR. In some embodiments, the 3′UTR is a GJB2 gene exon 2 3′ UTR. In some embodiments, the nucleotide sequence encoding the 3′ UTR has at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 32. An exemplary nucleotide sequence encoding GJB2 gene exon 2 3′ UTR is set forth in SEQ ID NO: 32:

CGCATTGCCCAGTTGTTAGATTAAGAAATAGACAGCATGAGAGGGATGAG
GCAACCCGTGCTCAGCTGTCAAGGCTCAGTCGCTAGCATTTCCCAACACA
AAGATTCTGACCTTAAATGCAACCATTTGAAACCCCTGTAGGCCTCAGGT
GAAACTCCAGATGCCACAATGGAGCTCTGCTCCCCTAAAGCCTCAAAACA
AAGGCCTAATTCTATGCCTGTCTTAATTTTCTTTCACTTAAGTTAGTTCC
ACTGAGACCCCAGGCTGTTAGGGGTTATTGGTGTAAGGTACTTTCATATT
TTAAACAGAGGATATCGGCATTTGTTTCTTTCTCTGAGGACAAGAGAAAA
AAGCCAGGTTCCACAGAGGACACAGAGAAGGTTTGGGTGTCCTCCTGGGG
TTCTTTTTGCCAACTTTCCCCACGTTAAAGGTGAACATTGGTTCTTTCAT
TTGCTTTGGAAGTTTTAATCTCTAACAGTGGACAAAGTTACCAGTGCCTT
AAACTCTGTTACACTTTTTGGAAGTGAAAACTTTGTAGTATGATAGGTTA
TTTTGATGTAAAGATGTTCTGGATACCATTATATGTTCCCCCTGTTTCAG
AGGCTCAGATTGTAATATGTAAATGGTATGTCATTCGCTACTATGATTTA
ATTTGAAATATGGTCTTTTGGTTATGAATACTTTGCAGCACAGCTGAGAG
GCTGTCTGTTGTATTCATTGTGGTCATAGCACCTAACAACATTGTAGCCT
CAATCGAGTGAGACAGACTAGAAGTTCCTAGTGATGGCTTATGATAGCAA
ATGGCCTCATGTCAAATATTTAGATGTAATTTTGTGTAAGAAATACAGAC
TGGATGTACCACCAACTACTACCTGTAATGACAGGCCTGTCCAACACATC
TCCCTTTTCCATGACTGTGGTAGCCAGCATCGGAAAGAACGCTGATTTAA
AGAGGTCGCTTGGGAATTTTATTGACACAGTACCATTTAATGGGGAGGAC
AAAATGGGGCAGGGGAGGGAGAAGTTTCTGTCGTTAAAAACAGATTTGGA
AAGACTGGACTCTAAAGTCTGTTGATTAAAGATGAGCTTTGTCTACTTCA
AAAGTTTGTTTGCTTACCCCTTCAGCCTCCAATTTTTTAAGTGAAAATAT
AGCTAATAACATGTGAAAAGAATAGAAGCTAAGGTTTAGATAAATATTGA
GCAGATCTATAGGAAGATTGAACCTGAATATTGCCATTATGCTTGACATG
GTTTCCAAAAAATGGTACTCCACATATTTCAGTGAGGGTAAGTATTTTCC
TGTTGTCAAGAATAGCATTGTAAAAGCATTTTGTAATAATAAAGAATAGC
TTTAATGATATGCTTGTAACTAAAATAATTTTGTAATGTATCAAATACAT
TTAAAACATTAAAATATAATCTCTATAATAA

In some embodiments, the expression cassette of the isolated nucleic acid comprises a de-targeting agent that restricts or reduces the transgene expression (e.g., GJB2 expression) in a cell type (e.g., hair cell or spiral ganglion neurons). In some embodiments, incorporation of one or more miRNA binding sites into an expression allows for de-targeting of transgene expression in a cell-type specific manner (e.g., in hair cell or spiral ganglion neurons). In some embodiments, one or more miRNA binding sites are positioned in the 3′ UTR (e.g., GJB2 exon 2 3′ UTR of the expression cassette of the isolated nucleic acid).

In some embodiments, an expression cassette comprises one or more (e.g., 1, 2, 3, 4, 5, or more) miRNA binding sites that de-target expression of GJB2 from cells that do not normally express GJB2 (e.g., hair cell or spiral ganglion neurons). In some embodiments, the expression cassette of the isolated nucleic acid comprises one or more miR binding sites for detargeting neuron cells (e.g., spiral ganglion neurons), e.g., binding sites for neuron enriched miRs as described in Jovičić et al., Comprehensive Expression Analyses of Neural Cell-Type-Specific miRNAs Identify New Determinants of the Specification and Maintenance of Neuronal Phenotypes, J Neurosci. 2013 Mar. 20; 33(12): 5127-5137, which is incorporated herein by reference. Non-limiting examples of neuron enriched miRs include miR-124, miR-127, miR-129, miR-129*, miR-136, miR-136*, miR-137, miR-154, miR-300-3p, miR-323, miR-329, miR-341, miR-369-5p, miR-376a, miR-376b-3p, miR-376c, miR-379, miR-382, miR-382*, miR-410, miR-411, miR-433, miR-434, miR-495, miR-541, miR-543*, miR-551b, miR-143, miR-449a, miR-219-2-3p, miR-126, miR-126*, miR-141, miR-142-3p, miR-142-5p, miR-146a, miR-150, miR-200c, or miR-223. In some embodiments, the expression cassette of the isolated nucleic acid comprises one or more miR binding sites for detargeting hair cells (e.g., inner or outer hair cell), e.g., binding sites for hair cell enriched miRs as described in Li et al., MicroRNAs in hair cell development and deafness, Curr Opin Otolaryngol Head Neck Surg. 2010 October; 18(5): 459-465, which is incorporated herein by reference. Non-limiting examples of neuron enriched miRs include miR-96, miR-182, miR-183, miR-18a, or miR-99a. In some embodiments, the GJB2 exon 2 3′ UTR of the expression cassette comprises one or more miR binding sites for detargeting neuron cells and hair cells. In some embodiments, the GJB2 exon 2 3′ UTR of the expression cassette comprises one or more miR binding sites for miR-124.

Aspects of the disclosure relate to gene therapy vectors comprising an isolated nucleic acid as described herein. A gene therapy vector may be a viral vector (e.g., a lentiviral vector, an adeno-associated virus vector, an adenoviral (Ad) vector, etc.), a plasmid, a closed-ended DNA (e.g., ceDNA), a lipid/DNA nanoparticle, etc. In some embodiments, a gene therapy vector is a viral vector. In some embodiments, an expression cassette encoding a protein (e.g., GJB2 protein) is flanked by one or more viral replication sequences, for example, lentiviral long terminal repeats (LTRs) or adeno-associated virus (AAV) inverted terminal repeats (ITRs).

The isolated nucleic acids of the disclosure may be recombinant adeno-associated virus (AAV) vectors (rAAV vectors). In some embodiments, an isolated nucleic acid as described by the disclosure comprises two adeno-associated virus (AAV) inverted terminal repeat (ITR) sequences, or variants thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, an expression cassette (e.g., expression cassette for GJB2), and 5′ and 3′ AAV inverted terminal repeats (ITRs). The isolated nucleic acids may also comprise a region encoding, for example, 5′ and 3′ untranslated regions (UTRs), and/or an expression control sequence (e.g., a poly-A tail).

Generally, ITR sequences are about 145 bp in length. Preferably, substantially the entire sequence encoding the ITR is used in the isolated nucleic acid, although some degree of minor modification of these sequences is permissible. The ability to modify these ITR sequences is within the skill of one in the art. (See, e.g., texts such as Sambrook et al., Molecular Cloning. A Laboratory Manual, 2d ed., Cold Spring Harbor Laboratory, New York (1989); and K. Fisher et al., J Virol., 70:520 532 (1996)). An example of such a molecule employed in the present invention is an isolated nucleic acid comprising an expression cassette encoding a GJB2 protein, in which the expression cassette comprising the nucleotide sequences GJB2 protein and GJB2 gene regulatory elements (GREs) are flanked by the 5′ and 3′ AAV ITR sequences. The AAV ITR sequences may be obtained from any known AAV, including presently identified mammalian AAV types. In some embodiments, the isolated nucleic acid (e.g., the rAAV vector) comprises at least one ITR having a serotype selected from AAV1, AAV2, AAV5, AAV6, AAV6.2, AAV7, AAV8, AAV9, AAV10, AAV11, and variants thereof. In some embodiments, the isolated nucleic acid comprises a region (e.g., a first region) encoding an AAV2 ITR.

In some embodiments, the isolated nucleic acid further comprises a region (e.g., a second region, a third region, a fourth region, etc.) comprising a second AAV ITR. In some embodiments, the second AAV ITR has a serotype selected from AAV1, AAV2, AAV5, AAV6, AAV6.2, AAV7, AAV8, AAV9, AAV10, AAV11, and variants thereof. In some embodiments, the second AAV ITR is an AAV2 ITR. In some embodiments, the second ITR is a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ATRS ITR, or AITR). Without wishing to be bound by any particular theory, an rAAV vector comprising an ITR lacking a functional TRS produces a self-complementary rAAV vector, for example, as described by McCarthy (2008) Molecular Therapy 16(10):1648-1656. In some embodiments, the isolated nucleic acid comprises a 5′ AAV2 ITR and a 3′ AAV2 ITR.

An exemplary 5′ AAV2 ITR nucleotide sequence is set forth in SEQ ID NO: 34:

TTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGC
GAGCGCGCAGAGAGGGAGTGGCCAACTCCATCACTAGGGGTTCCTAGAT

An exemplary 5′ ITR nucleotide sequence is set forth in SEQ ID NO: 106:

CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCT

exemplary 3′ AAV2 ITR nucleotide sequence is set forth in SEQ ID NO: 35:

CCCCTAGTGATGGAGTTGGCCACTCCCTCTCTGCGCGCTCGCTCGCTCAC
TGAGGCCGCCCGGGCAAAGCCCGGGCGTCGGGCGACCTTTGGTCGCCCGG
CCTCAGTGAGCGAGCGAGCGCGCAGAGAGGGAGTGGCCA

An exemplary 3′ ITR nucleotide sequence is set forth in SEQ ID NO: 107:

AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAG

In some embodiments, the isolated nucleic acid (e.g., rAAV vector) described herein comprises a 5′ ITR sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 34 or 106.

In some embodiments, the isolated nucleic acid (e.g., rAAV vector) described herein comprises a 3′ ITR sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 35 or 107.

In some embodiments, the isolated nucleic acid (e.g., rAAV vector) described herein comprises a posttranscriptional response element. As used herein, the term “posttranscriptional response element” refers to a nucleic acid sequence that, when transcribed, adopts a tertiary structure that enhances expression of a gene. Examples of posttranscriptional regulatory elements include, but are not limited to, woodchuck hepatitis virus posttranscriptional regulatory element (WPRE), mouse RNA transport element (RTE), constitutive transport element (CTE) of the simian retrovirus type 1 (SRV-1), the CTE from the Mason-Pfizer monkey virus (MPMV), and the 5′ untranslated region of the human heat shock protein 70 (Hsp70 5′ UTR). In some embodiments, the isolated nucleic acid (e.g., rAAV vector) comprises a woodchuck hepatitis virus posttranscriptional regulatory element (WPRE).

In some embodiments, the isolated nucleic acid (e.g., rAAV vector) described herein comprises a posttranscriptional response element having at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 108. An exemplary posttranscriptional response element is set forth in SEQ ID NO: 108:

GATAATCAACCTCTGGATTACAAAATTTGTGAAAGATTGACTGGTATTCT
TAACTATGTTGCTCCTTTTACGCTATGTGGATACGCTGCTTTAATGCCTT
TGTATCATGCTATTGCTTCCCGTATGGCTTTCATTTTCTCCTCCTTGTAT
AAATCCTGGTTGCTGTCTCTTTATGAGGAGTTGTGGCCCGTTGTCAGGCA
ACGTGGCGTGGTGTGCACTGTGTTTGCTGACGCAACCCCCACTGGTTGGG
GCATTGCCACCACCTGTCAGCTCCTTTCCGGGACTTTCGCTTTCCCCCTC
CCTATTGCCACGGCGGAACTCATCGCCGCCTGCCTTGCCCGCTGCTGGAC
AGGGGCTCGGCTGTTGGGCACTGACAATTCCGTGGTGTTGTCGGGGAAAT
CATCGTCCTTTCCTTGGCTGCTCGCCTGTGTTGCCACCTGGATTCTGCGC
GGGACGTCCTTCTGCTACGTCCCTTCGGCCCTCAATCCAGCGGACCTTCC
TTCCCGCGGCCTGCTGCCGGCTCTGCGGCCTCTTCCGCGTCTTCGCCTTC
GCCCTCAGACGAGTCGGATCTCCCTTTGGGCCGCCTCCCCGCATCGGACT
AG

In some embodiments, the vector further comprises conventional control elements which are operably linked with elements of the GJB2 coding sequence in a manner that permits its transcription, translation, and/or expression in a cell transfected with the vector or infected with the virus produced by the disclosure. Expression control sequences include appropriate transcription initiation, termination; efficient RNA processing signals, such as splicing and polyadenylation (polyA) signals; sequences that stabilize cytoplasmic mRNA; sequences that enhance translation efficiency (e.g., Kozak consensus sequence); sequences that enhance protein stability. A polyadenylation sequence generally is inserted following the coding sequences and optionally before a 3′ AAV ITR sequence. A rAAV construct useful in the disclosure may also contain an intron, desirably located between the promoter/enhancer sequence and the transgene.

In some embodiments, the isolated nucleic acid (e.g., rAAV vector) described herein comprises a polyadenylation signal sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 109. An exemplary polyadenylation signal sequence is set forth in SEQ ID NO: 109:

GTCGACTAGAGCTCGCTGATCAGCCTCGACTGTGCCTTCTAGTTGCCAGC
CATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCT
GAGTAGGIGTCATTCTATTCTGGGGGGTGGGGTGGGGCAGGACAGCAAGG
GGGAGGATTGGGAAGACAATAGCAGGCATGCTGGGGA

In some embodiments, an AAV vector described herein comprises a GJB2 proximal promoter (e.g., SEQ ID NO: 102), a GJB2 5′ UTR (e.g., SEQ ID NO: 103 and CC), a nucleotide sequence encoding a GJB2 gene product (e.g., SEQ ID NO: 2), a GJB2 3′ UTR (e.g., SEQ ID NO: 32), a WPRE (e.g., SEQ ID NO: 108), and a bovine growth hormone poly A signal (e.g., SEQ ID NO: 109). In some embodiments, an AAV vector described herein comprises a nucleotide sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 110. An exemplary AAV vector sequence is set forth in SEQ ID NO: 110:

GACCTCGAAGGGGACTTGGGGGGTTCGGGGCTTTCGGGGGCGGTCGGGGGTTCGCGGACCCG
GGAAGCTCTGAGGACCCAGAGGCCGGGCGCGCTCCGCCCGCGGCGCCGCCCCCTCCGTAACT
TTCCCAGTCTCCGAGGGAAGAGGCGGGGTGTGGGGTGCGGTTAAAAGGCGCCACGGCGGGAG
ACAGGTGTTGCGGCCCCGCAGCGCCCGCGCGCTCCTCTCCCCGACTCGGAGCCCCTCGGCGG
CGCCCGGCCCAGGACCCGCCTAGGAGCGCAGGAGCCCCAGCGCAGAGACCCCAACGCCGAGA
CCCCCGCCCCGGCCCCGCCGCGCTTCCTCCCGACGCAGAGCAAACCGCCCAGAGTAGAAGCG
GATCCGCCACCATGGATTGGGGCACGCTGCAGACGATCCTGGGGGGTGTGAACAAACACTCC
ACCAGCATTGGAAAGATCTGGCTCACCGTCCTCTTCATTTTTCGCATTATGATCCTCGTTGT
GGCTGCAAAGGAGGTGTGGGGAGATGAGCAGGCCGACTTTGTCTGCAACACCCTGCAGCCAG
GCTGCAAGAACGTGTGCTACGATCACTACTTCCCCATCTCCCACATCCGGCTATGGGCCCTG
CAGCTGATCTTCGTGTCCACGCCAGCGCTCCTAGTGGCCATGCACGTGGCCTACCGGAGACA
TGAGAAGAAGAGGAAGTTCATCAAGGGGGAGATAAAGAGTGAATTTAAGGACATCGAGGAGA
TCAAAACCCAGAAGGTCCGCATCGAAGGCTCCCTGTGGTGGACCTACACAAGCAGCATCTTC
TTCCGGGTCATCTTCGAAGCCGCCTTCATGTACGTCTTCTATGTCATGTACGACGGCTTCTC
CATGCAGCGGCTGGTGAAGTGCAACGCCTGGCCTTGTCCCAACACTGTGGACTGCTTTGTGT
CCCGGCCCACGGAGAAGACTGTCTTCACAGTGTTCATGATTGCAGTGTCTGGAATTTGCATC
CTGCTGAATGTCACTGAATTGTGTTATTTGCTAATTAGATATTGTTCTGGGAAGTCAAAAAA
GCCAGTTTACCCATACGATGTTCCAGATTACGCTTAAGGCGCGCCACCCCTGCAGGGAATTC
CGCATTGCCCAGTTGTTAGATTAAGAAATAGACAGCATGAGAGGGATGAGGCAACCCGTGCT
CAGCTGTCAAGGCTCAGTCGCTAGCATTTCCCAACACAAAGATTCTGACCTTAAATGCAACC
ATTTGAAACCCCTGTAGGCCTCAGGTGAAACTCCAGATGCCACAATGGAGCTCTGCTCCCCT
AAAGCCTCAAAACAAAGGCCTAATTCTATGCCTGTCTTAATTTTCTTTCACTTAAGTTAGTT
CCACTGAGACCCCAGGCTGTTAGGGGTTATTGGTGTAAGGTACTTTCATATTTTAAACAGAG
GATATCGGCATTTGTTTCTTTCTCTGAGGACAAGAGAAAAAAGCCAGGTTCCACAGAGGACA
CAGAGAAGGTTTGGGTGTCCTCCTGGGGTTCTTTTTGCCAACTTTCCCCACGTTAAAGGTGA
ACATTGGTTCTTTCATTTGCTTTGGAAGTTTTAATCTCTAACAGTGGACAAAGTTACCAGTG
CCTTAAACTCTGTTACACTTTTTGGAAGTGAAAACTTTGTAGTATGATAGGTTATTTTGATG
TAAAGATGTTCTGGATACCATTATATGTTCCCCCTGTTTCAGAGGCTCAGATTGTAATATGT
AAATGGTATGTCATTCGCTACTATGATTTAATTTGAAATATGGTCTTTTGGTTATGAATACT
TTGCAGCACAGCTGAGAGGCTGTCTGTTGTATTCATTGTGGTCATAGCACCTAACAACATTG
TAGCCTCAATCGAGTGAGACAGACTAGAAGTTCCTAGTGATGGCTTATGATAGCAAATGGCC
TCATGTCAAATATTTAGATGTAATTTTGTGTAAGAAATACAGACTGGATGTACCACCAACTA
CTACCTGTAATGACAGGCCTGTCCAACACATCTCCCTTTTCCATGACTGTGGTAGCCAGCAT
CGGAAAGAACGCTGATTTAAAGAGGTCGCTTGGGAATTTTATTGACACAGTACCATTTAATG
GGGAGGACAAAATGGGGCAGGGGAGGGAGAAGTTTCTGTCGTTAAAAACAGATTTGGAAAGA
CTGGACTCTAAAGTCTGTTGATTAAAGATGAGCTTTGTCTACTTCAAAAGTTTGTTTGCTTA
CCCCTTCAGCCTCCAATTTTTTAAGTGAAAATATAGCTAATAACATGTGAAAAGAATAGAAG
CTAAGGTTTAGATAAATATTGAGCAGATCTATAGGAAGATTGAACCTGAATATTGCCATTAT
GCTTGACATGGTTTCCAAAAAATGGTACTCCACATATTTCAGTGAGGGTAAGTATTTTCCTG
TTGTCAAGAATAGCATTGTAAAAGCATTTTGTAATAATAAAGAATAGCTTTAATGATATGCT
TGTAACTAAAATAATTTTGTAATGTATCAAATACATTTAAAACATTAAAATATAATCTCTAT
AATAATTTAAAATCTAATATGGTTTTAATAGAACAGCGATATCAAGCTTATCGATGATAATC
AACCTCTGGATTACAAAATTTGTGAAAGATTGACTGGTATTCTTAACTATGTTGCTCCTTTT
ACGCTATGTGGATACGCTGCTTTAATGCCTTTGTATCATGCTATTGCTTCCCGTATGGCTTT
CATTTTCTCCTCCTTGTATAAATCCTGGTTGCTGTCTCTTTATGAGGAGTTGTGGCCCGTTG
TCAGGCAACGTGGCGTGGTGTGCACTGTGTTTGCTGACGCAACCCCCACTGGTTGGGGCATT
GCCACCACCTGTCAGCTCCTTTCCGGGACTTTCGCTTTCCCCCTCCCTATTGCCACGGCGGA
ACTCATCGCCGCCTGCCTTGCCCGCTGCTGGACAGGGGCTCGGCTGTTGGGCACTGACAATT
CCGTGGTGTTGTCGGGGAAATCATCGTCCTTTCCTTGGCTGCTCGCCTGTGTTGCCACCTGG
ATTCTGCGCGGGACGTCCTTCTGCTACGTCCCTTCGGCCCTCAATCCAGCGGACCTTCCTTC
CCGCGGCCTGCTGCCGGCTCTGCGGCCTCTTCCGCGTCTTCGCCTTCGCCCTCAGACGAGTC
GGATCTCCCTTTGGGCCGCCTCCCCGCATCGGACTAGGAATTCATCGATACCGAGCGCTGCT
CGAGAGATCTGTGATAGCGGCCATCAAGCTGGGTCGACTAGAGCTCGCTGATCAGCCTCGAC
TGTGCCTTCTAGTTGCCAGCCATCTGTTGTTTGCCCCTCCCCCGTGCCTTCCTTGACCCTGG
AAGGTGCCACTCCCACTGTCCTTTCCTAATAAAATGAGGAAATTGCATCGCATTGTCTGAGT
AGGTGTCATTCTATTCTGGGGGGTGGGGTGGGGCAGGACAGCAAGGGGGAGGATTGGGAAGA
CAATAGCAGGCATGCTGGGGA

In some embodiments, an AAV vector described herein comprises a 5′ ITR (e.g., SEQ ID NO: 106), a GJB2 proximal promoter (e.g., SEQ ID NO: 102), a GJB2 5′ UTR (e.g., SEQ ID NO: 103 and CC), a nucleotide sequence encoding a GJB2 gene product (e.g., SEQ ID NO: 2), a GJB2 3′ UTR (e.g., SEQ ID NO: 32), a WPRE (e.g., SEQ ID NO: 108), a bovine growth hormong poly A signal (e.g., SEQ ID NO: 109), and a 3′ ITR (e.g., SEQ ID NO: 107). In some embodiments, an AAV vector described herein comprises a nucleotide sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 111. An exemplary AAV vector sequence is set forth in SEQ ID NO: 111:

CTGCGCGCTCGCTCGCTCACTGAGGCCGCCCGGGCAAAGCCCGGGCGTCGGGCGACCTTTGG
TCGCCCGGCCTCAGTGAGCGAGCGAGCGCGCAGAGAGGGAGTGGCCAACTCCATCACTAGGG
GTTCCTTGTAGTTAATGATTAACCCGCCATGCTACTTATCTACCAGGGTAATGGGGATCCTC
TAGAACGCGTTTAATTAAGACCTCGAAGGGGACTTGGGGGGTTCGGGGCTTTCGGGGGCGGT
CGGGGGTTCGCGGACCCGGGAAGCTCTGAGGACCCAGAGGCCGGGCGCGCTCCGCCCGCGGC
GCCGCCCCCTCCGTAACTTTCCCAGTCTCCGAGGGAAGAGGCGGGGTGTGGGGTGCGGTTAA
AAGGCGCCACGGCGGGAGACAGGTGTTGCGGCCCCGCAGCGCCCGCGCGCTCCTCTCCCCGA
CTCGGAGCCCCTCGGCGGCGCCCGGCCCAGGACCCGCCTAGGAGCGCAGGAGCCCCAGCGCA
GAGACCCCAACGCCGAGACCCCCGCCCCGGCCCCGCCGCGCTTCCTCCCGACGCAGAGCAAA
CCGCCCAGAGTAGAAGCGGATCCGCCACCATGGATTGGGGCACGCTGCAGACGATCCTGGGG
GGTGTGAACAAACACTCCACCAGCATTGGAAAGATCTGGCTCACCGTCCTCTTCATTTTTCG
CATTATGATCCTCGTTGTGGCTGCAAAGGAGGTGTGGGGAGATGAGCAGGCCGACTTTGTCT
GCAACACCCTGCAGCCAGGCTGCAAGAACGTGTGCTACGATCACTACTTCCCCATCTCCCAC
ATCCGGCTATGGGCCCTGCAGCTGATCTTCGTGTCCACGCCAGCGCTCCTAGTGGCCATGCA
CGTGGCCTACCGGAGACATGAGAAGAAGAGGAAGTTCATCAAGGGGGAGATAAAGAGTGAAT
TTAAGGACATCGAGGAGATCAAAACCCAGAAGGTCCGCATCGAAGGCTCCCTGTGGTGGACC
TACACAAGCAGCATCTTCTTCCGGGTCATCTTCGAAGCCGCCTTCATGTACGTCTTCTATGT
CATGTACGACGGCTTCTCCATGCAGCGGCTGGTGAAGTGCAACGCCTGGCCTTGTCCCAACA
CTGTGGACTGCTTTGTGTCCCGGCCCACGGAGAAGACTGTCTTCACAGTGTTCATGATTGCA
GTGTCTGGAATTTGCATCCTGCTGAATGTCACTGAATTGTGTTATTTGCTAATTAGATATTG
TTCTGGGAAGTCAAAAAAGCCAGTTTACCCATACGATGTTCCAGATTACGCTTAAGGCGCGC
CACCCCTGCAGGGAATTCCGCATTGCCCAGTTGTTAGATTAAGAAATAGACAGCATGAGAGG
GATGAGGCAACCCGTGCTCAGCTGTCAAGGCTCAGTCGCTAGCATTTCCCAACACAAAGATT
CTGACCTTAAATGCAACCATTTGAAACCCCTGTAGGCCTCAGGTGAAACTCCAGATGCCACA
ATGGAGCTCTGCTCCCCTAAAGCCTCAAAACAAAGGCCTAATTCTATGCCTGTCTTAATTTT
CTTTCACTTAAGTTAGTTCCACTGAGACCCCAGGCTGTTAGGGGTTATTGGTGTAAGGTACT
TTCATATTTTAAACAGAGGATATCGGCATTTGTTTCTTTCTCTGAGGACAAGAGAAAAAAGC
CAGGTTCCACAGAGGACACAGAGAAGGTTTGGGTGTCCTCCTGGGGTTCTTTTTGCCAACTT
TCCCCACGTTAAAGGTGAACATTGGTTCTTTCATTTGCTTTGGAAGTTTTAATCTCTAACAG
TGGACAAAGTTACCAGTGCCTTAAACTCTGTTACACTTTTTGGAAGTGAAAACTTTGTAGTA
TGATAGGTTATTTTGATGTAAAGATGTTCTGGATACCATTATATGTTCCCCCTGTTTCAGAG
GCTCAGATTGTAATATGTAAATGGTATGTCATTCGCTACTATGATTTAATTTGAAATATGGT
CTTTTGGTTATGAATACTTTGCAGCACAGCTGAGAGGCTGTCTGTTGTATTCATTGTGGTCA
TAGCACCTAACAACATTGTAGCCTCAATCGAGTGAGACAGACTAGAAGTTCCTAGTGATGGC
TTATGATAGCAAATGGCCTCATGTCAAATATTTAGATGTAATTTTGTGTAAGAAATACAGAC
TGGATGTACCACCAACTACTACCTGTAATGACAGGCCTGTCCAACACATCTCCCTTTTCCAT
GACTGTGGTAGCCAGCATCGGAAAGAACGCTGATTTAAAGAGGTCGCTTGGGAATTTTATTG
ACACAGTACCATTTAATGGGGAGGACAAAATGGGGCAGGGGAGGGAGAAGTTTCTGTCGTTA
AAAACAGATTTGGAAAGACTGGACTCTAAAGTCTGTTGATTAAAGATGAGCTTTGTCTACTT
CAAAAGTTTGTTTGCTTACCCCTTCAGCCTCCAATTTTTTAAGTGAAAATATAGCTAATAAC
ATGTGAAAAGAATAGAAGCTAAGGTTTAGATAAATATTGAGCAGATCTATAGGAAGATTGAA
CCTGAATATTGCCATTATGCTTGACATGGTTTCCAAAAAATGGTACTCCACATATTTCAGTG
AGGGTAAGTATTTTCCTGTTGTCAAGAATAGCATTGTAAAAGCATTTTGTAATAATAAAGAA
TAGCTTTAATGATATGCTTGTAACTAAAATAATTTTGTAATGTATCAAATACATTTAAAACA
TTAAAATATAATCTCTATAATAATTTAAAATCTAATATGGTTTTAATAGAACAGCGATATCA
AGCTTATCGATGATAATCAACCTCTGGATTACAAAATTTGTGAAAGATTGACTGGTATTCTT
AACTATGTTGCTCCTTTTACGCTATGTGGATACGCTGCTTTAATGCCTTTGTATCATGCTAT
TGCTTCCCGTATGGCTTTCATTTTCTCCTCCTTGTATAAATCCTGGTTGCTGTCTCTTTATG
AGGAGTTGTGGCCCGTTGTCAGGCAACGTGGCGTGGTGTGCACTGTGTTTGCTGACGCAACC
CCCACTGGTTGGGGCATTGCCACCACCTGTCAGCTCCTTTCCGGGACTTTCGCTTTCCCCCT
CCCTATTGCCACGGCGGAACTCATCGCCGCCTGCCTTGCCCGCTGCTGGACAGGGGCTCGGC
TGTTGGGCACTGACAATTCCGTGGTGTTGTCGGGGAAATCATCGTCCTTTCCTTGGCTGCTC
GCCTGTGTTGCCACCTGGATTCTGCGCGGGACGTCCTTCTGCTACGTCCCTTCGGCCCTCAA
TCCAGCGGACCTTCCTTCCCGCGGCCTGCTGCCGGCTCTGCGGCCTCTTCCGCGTCTTCGCC
TTCGCCCTCAGACGAGTCGGATCTCCCTTTGGGCCGCCTCCCCGCATCGGACTAGGAATTCA
TCGATACCGAGCGCTGCTCGAGAGATCTGTGATAGCGGCCATCAAGCTGGGTCGACTAGAGC
TCGCTGATCAGCCTCGACTGTGCCTTCTAGTTGCCAGCCATCTGTTGTTTGCCCCTCCCCCG
TGCCTTCCTTGACCCTGGAAGGTGCCACTCCCACTGTCCTTTCCTAATAAAATGAGGAAATT
GCATCGCATTGTCTGAGTAGGTGTCATTCTATTCTGGGGGGTGGGGTGGGGCAGGACAGCAA
GGGGGAGGATTGGGAAGACAATAGCAGGCATGCTGGGGACACGTGCGGACCGAGCGGCCGCG
GTACCAAACCTAGGTAATACCCATTACCCTGGTAGATAAGTAGCATGGCGGGTTAATCATTA
ACTACAAGGAACCCCTAGTGATGGAGTTGGCCACTCCCTCTCTGCGCGCTCGCTCGCTCACT
GAGGCCGGGCGACCAAAGGTCGCCCGACGCCCGGGCTTTGCCCGGGCGGCCTCAGTGAGCGA
GCGAGCGCGCAG

In some embodiments, an AAV vector described herein comprises 5′ ITR, a GJB2 basal promoter, a 5′ UTR (e.g., GJB2 exon 1 5′ UTR), Kozak sequence, nucleotide sequence encoding a gene product (e.g., GJB2 or GFP), an optional HA tag, a 3′ UTR (e.g., GJB2 exon 2 3′ UTR), a WPRE, a bovine growth hormone poly A signal, and a 3′ ITR (e.g., vector c70). In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 36. An exemplary nucleotide sequence for vector c70 encoding a mouse GJB2 protein with an HA tag is set forth in SEQ ID NO: 36 (mouse GJB2 coding sequence in boldface; HA tag underlined):

TTGCTGGCCTTTTGCTCACATGTCCTGCAGGCAGCTGCGCGCTCGCTCGCTCACTGAGGCCG
CCCGGGCAAAGCCCGGGCGTCGGGCGACCTTTGGTCGCCCGGCCTCAGTGAGCGAGCGAGCG
CGCAGAGAGGGAGTGGCCAACTCCATCACTAGGGGTTCCTGCGGCCGCACGCGTTTAATTAA
GACCTCGAAGGGGACTTGGGGGGTTCGGGGCTTTCGGGGGCGGTCGGGGGTTCGCGGACCCG
GGAAGCTCTGAGGACCCAGAGGCCGGGCGCGCTCCGCCCGCGGCGCCGCCCCCTCCGTAACT
TTCCCAGTCTCCGAGGGAAGAGGCGGGGTGTGGGGTGCGGTTAAAAGGCGCCACGGCGGGAG
ACAGGTGTTGCGGCCCCGCAGCGCCCGCGCGCTCCTCTCCCCGACTCGGAGCCCCTCGGCGG
CGCCCGGCCCAGGACCCGCCTAGGAGCGCAGGAGCCCCAGCGCAGAGACCCCAACGCCGAGA
CCCCCGCCCCGGCCCCGCCGCGCTTCCTCCCGACGCAGAGCAAACCGCCCAGAGTAGAAGCG
GATCCGCCACCATGGATTGGGGCACACTCCAGAGCATCCTCGGGGGTGTCAACAAACACTCC
ACCAGCATTGGAAAGATCTGGCTCACGGTCCTCTTCATCTTCCGCATCATGATCCTCGTGGT
GGCTGCAAAGGAGGTGTGGGGAGATGAGCAAGCCGATTTTGTCTGCAACACGCTCCAGCCTG
GCTGCAAGAATGTATGCTACGACCACCACTTCCCCATCTCTCACATCCGGCTCTGGGCTCTG
CAGCTGATCATGGTGTCCACGCCAGCCCTCCTGGTAGCTATGCATGTGGCCTACCGGAGACA
TGAAAAGAAACGGAAGTTCATGAAGGGAGAGATAAAGAACGAGTTTAAGGACATCGAAGAGA
TCAAAACCCAGAAGGTCCGTATCGAAGGGTCCCTGTGGTGGACCTACACCACCAGCATCTTC
TTCCGGGTCATCTTTGAAGCCGTCTTCATGTACGTCTTTTACATCATGTACAATGGCTTCTT
CATGCAACGTCTGGTGAAATGCAACGCTTGGCCCTGCCCCAATACAGTGGACTGCTTCATTT
CCAGGCCCACAGAAAAGACTGTCTTCACCGTGTTTATGATTTCTGTGTCTGGAATTTGCATT
CTGCTAAATATCACAGAGCTGTGCTATTTGTTCGTTAGGTATTGCTCAGGAAAGTCCAAAAG
ACCAGTCTACCCATACGATGTTCCAGATTACGCTTAAGGCGCGCCACCCCTGCAGGGAATTC
CGCATTGCCCAGTTGTTAGATTAAGAAATAGACAGCATGAGAGGGATGAGGCAACCCGTGCT
CAGCTGTCAAGGCTCAGTCGCTAGCATTTCCCAACACAAAGATTCTGACCTTAAATGCAACC
ATTTGAAACCCCTGTAGGCCTCAGGTGAAACTCCAGATGCCACAATGGAGCTCTGCTCCCCT
AAAGCCTCAAAACAAAGGCCTAATTCTATGCCTGTCTTAATTTTCTTTCACTTAAGTTAGTT
CCACTGAGACCCCAGGCTGTTAGGGGTTATTGGTGTAAGGTACTTTCATATTTTAAACAGAG
GATATCGGCATTTGTTTCTTTCTCTGAGGACAAGAGAAAAAAGCCAGGTTCCACAGAGGACA
CAGAGAAGGTTTGGGTGTCCTCCTGGGGTTCTTTTTGCCAACTTTCCCCACGTTAAAGGTGA
ACATTGGTTCTTTCATTTGCTTTGGAAGTTTTAATCTCTAACAGTGGACAAAGTTACCAGTG
CCTTAAACTCTGTTACACTTTTTGGAAGTGAAAACTTTGTAGTATGATAGGTTATTTTGATG
TAAAGATGTTCTGGATACCATTATATGTTCCCCCTGTTTCAGAGGCTCAGATTGTAATATGT
AAATGGTATGTCATTCGCTACTATGATTTAATTTGAAATATGGTCTTTTGGTTATGAATACT
TTGCAGCACAGCTGAGAGGCTGTCTGTTGTATTCATTGTGGTCATAGCACCTAACAACATTG
TAGCCTCAATCGAGTGAGACAGACTAGAAGTTCCTAGTGATGGCTTATGATAGCAAATGGCC
TCATGTCAAATATTTAGATGTAATTTTGTGTAAGAAATACAGACTGGATGTACCACCAACTA
CTACCTGTAATGACAGGCCTGTCCAACACATCTCCCTTTTCCATGACTGTGGTAGCCAGCAT
CGGAAAGAACGCTGATTTAAAGAGGTCGCTTGGGAATTTTATTGACACAGTACCATTTAATG
GGGAGGACAAAATGGGGCAGGGGAGGGAGAAGTTTCTGTCGTTAAAAACAGATTTGGAAAGA
CTGGACTCTAAAGTCTGTTGATTAAAGATGAGCTTTGTCTACTTCAAAAGTTTGTTTGCTTA
CCCCTTCAGCCTCCAATTTTTTAAGTGAAAATATAGCTAATAACATGTGAAAAGAATAGAAG
CTAAGGTTTAGATAAATATTGAGCAGATCTATAGGAAGATTGAACCTGAATATTGCCATTAT
GCTTGACATGGTTTCCAAAAAATGGTACTCCACATATTTCAGTGAGGGTAAGTATTTTCCTG
TTGTCAAGAATAGCATTGTAAAAGCATTTTGTAATAATAAAGAATAGCTTTAATGATATGCT
TGTAACTAAAATAATTTTGTAATGTATCAAATACATTTAAAACATTAAAATATAATCTCTAT
AATAATTTAAAATCTAATATGGTTTTAATAGAACAGCGATATCAAGCTTATCGATAATCAAC
CTCTGGATTACAAAATTTGTGAAAGATTGACTGGTATTCTTAACTATGTTGCTCCTTTTACG
CTATGTGGATACGCTGCTTTAATGCCTTTGTATCATGCTATTGCTTCCCGTATGGCTTTCAT
TTTCTCCTCCTTGTATAAATCCTGGTTGCTGTCTCTTTATGAGGAGTTGTGGCCCGTTGTCA
GGCAACGTGGCGTGGTGTGCACTGTGTTTGCTGACGCAACCCCCACTGGTTGGGGCATTGCC
ACCACCTGTCAGCTCCTTTCCGGGACTTTCGCTTTCCCCCTCCCTATTGCCACGGCGGAACT
CATCGCCGCCTGCCTTGCCCGCTGCTGGACAGGGGCTCGGCTGTTGGGCACTGACAATTCCG
TGGTGTTGTCGGGGAAATCATCGTCCTTTCCTTGGCTGCTCGCCTATGTTGCCACCTGGATT
CTGCGCGGGACGTCCTTCTGCTACGTCCCTTCGGCCCTCAATCCAGCGGACCTTCCTTCCCG
CGGCCTGCTGCCGGCTCTGCGGCCTCTTCCGCGTCTTCGCCTTCGCCCTCAGACGAGTCGGA
TCTCCCTTTGGGCCGCCTCCCCGCGAATTCATCGATACCGAGCGCTGCTCGAGAGATCTGTG
ATAGCGGCCATCAAGCTGGCTGTGCCTTCTAGTTGCCAGCCATCTGTTGTTTGCCCCTCCCC
CGTGCCTTCCTTGACCCTGGAAGGTGCCACTCCCACTGTCCTTTCCTAATAAAATGAGGAAA
TTGCATCGCATTGTCTGAGTAGGTGTCATTCTATTCTGGGGGGTGGGGTGGGGCAGGACAGC
AAGGGGGAGGATTGGGAAGACAATAGCAGGCATGCTGGGGACACGTGCGGACCGAGCGGCCG
CAGGAACCCCTAGTGATGGAGTTGGCCACTCCCTCTCTGCGCGCTCGCTCGCTCACTGAGGC
CGGGCGACCAAAGGTCGCCCGACGCCCGGGCTTTGCCCGGGCGGCCTCAGTGAGCGAGCGAG
CGCGCAGCTGCCTGCAGGGGCGCCTGATGCGGTATTTTCTCCTTACGCATCTGTGCGGTATT
TCACACCGCATACGTCAAAGCAACCATAGTACGCGCCCTGTAGCGGCGCATTAAGCGCGGCG
GGTGTGGTGGTTACGCGCAGCGTGACCGCTACACTTGCCAGCGCCCTAGCGCCCGCTCCTTT
CGCTTTCTTCCCTTCCTTTCTCGCCACGTTCGCCGGCTTTCCCCGTCAAGCTCTAAATCGGG
GGCTCCCTTTAGGGTTCCGATTTAGTGCTTTACGGCACCTCGACCCCAAAAAACTTGATTTG
GGTGATGGTTCACGTAGTGGGCCATCGCCCTGATAGACGGTTTTTCGCCCTTTGACGTTGGA
GTCCACGTTCTTTAATAGTGGACTCTTGTTCCAAACTGGAACAACACTCAACCCTATCTCGG
GCTATTCTTTTGATTTATAAGGGATTTTGCCGATTTCGGCCTATTGGTTAAAAAATGAGCTG
ATTTAACAAAAATTTAACGCGAATTTTAACAAAATATTAACGTTTACAATTTTATGGTGCAC
TCTCAGTACAATCTGCTCTGATGCCGCATAGTTAAGCCAGCCCCGACACCCGCCAACACCCG
CTGACGCGCCCTGACGGGCTTGTCTGCTCCCGGCATCCGCTTACAGACAAGCTGTGACCGTC
TCCGGGAGCTGCATGTGTCAGAGGTTTTCACCGTCATCACCGAAACGCGCGAGACGAAAGGG
CCTCGTGATACGCCTATTTTTATAGGTTAATGTCATGATAATAATGGTTTCTTAGACGTCAG
GTGGCACTTTTCGGGGAAATGTGCGCGGAACCCCTATTTGTTTATTTTTCTAAATACATTCA
AATATGTATCCGCTCATGAGACAATAACCCTGATAAATGCTTCAATAATATTGAAAAAGGAA
GAGTATGAGTATTCAACATTTCCGTGTCGCCCTTATTCCCTTTTTTGCGGCATTTTGCCTTC
CTGTTTTTGCTCACCCAGAAACGCTGGTGAAAGTAAAAGATGCTGAAGATCAGTTGGGTGCA
CGAGTGGGTTACATCGAACTGGATCTCAACAGCGGTAAGATCCTTGAGAGTTTTCGCCCCGA
AGAACGTTTTCCAATGATGAGCACTTTTAAAGTTCTGCTATGTGGCGCGGTATTATCCCGTA
TTGACGCCGGGCAAGAGCAACTCGGTCGCCGCATACACTATTCTCAGAATGACTTGGTTGAG
TACTCACCAGTCACAGAAAAGCATCTTACGGATGGCATGACAGTAAGAGAATTATGCAGTGC
TGCCATAACCATGAGTGATAACACTGCGGCCAACTTACTTCTGACAACGATCGGAGGACCGA
AGGAGCTAACCGCTTTTTTGCACAACATGGGGGATCATGTAACTCGCCTTGATCGTTGGGAA
CCGGAGCTGAATGAAGCCATACCAAACGACGAGCGTGACACCACGATGCCTGTAGCAATGGC
AACAACGTTGCGCAAACTATTAACTGGCGAACTACTTACTCTAGCTTCCCGGCAACAATTAA
TAGACTGGATGGAGGCGGATAAAGTTGCAGGACCACTTCTGCGCTCGGCCCTTCCGGCTGGC
TGGTTTATTGCTGATAAATCTGGAGCCGGTGAGCGTGGGTCTCGCGGTATCATTGCAGCACT
GGGGCCAGATGGTAAGCCCTCCCGTATCGTAGTTATCTACACGACGGGGAGTCAGGCAACTA
TGGATGAACGAAATAGACAGATCGCTGAGATAGGTGCCTCACTGATTAAGCATTGGTAACTG
TCAGACCAAGTTTACTCATATATACTTTAGATTGATTTAAAACTTCATTTTTAATTTAAAAG
GATCTAGGTGAAGATCCTTTTTGATAATCTCATGACCAAAATCCCTTAACGTGAGTTTTCGT
TCCACTGAGCGTCAGACCCCGTAGAAAAGATCAAAGGATCTTCTTGAGATCCTTTTTTTCTG
CGCGTAATCTGCTGCTTGCAAACAAAAAAACCACCGCTACCAGCGGTGGTTTGTTTGCCGGA
TCAAGAGCTACCAACTCTTTTTCCGAAGGTAACTGGCTTCAGCAGAGCGCAGATACCAAATA
CTGTCCTTCTAGTGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAAGTCGTGTCTTAC
CGGGTTGGACTCAAGACGATAGTTACCGGATAAGGCGCAGCGGTCGGGCTGAACGGGGGGTT
CGTGCACACAGCCCAGCTTGGAGCGAACGACCTACACCGAACTGAGATACCTACAGCGTGAG
CTATGAGAAAGCGCCACGCTTCCCGAAGGGAGAAAGGCGGACAGGTATCCGGTAAGCGGCAG
GGTCGGAACAGGAGAGCGCACGAGGGAGCTTCCAGGGGGAAACGCCTGGTATCTTTATAGTC
CTGTCGGGTTTCGCCACCTCTGACTTGAGCGTCGATTTTTGTGATGCTCGTCAGGGGGGCGG
AGCCTATGGAAAAACGCCAGCAACGCGGCCTTTTTACGGTTCCTGGCCTT

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 61. An exemplary nucleotide sequence for vector c70 encoding a human GJB2 protein with an HA tag is set forth in SEQ ID NO: 61 (human GJB2 coding sequence in boldface; HA tag underlined):

TTGCTGGCCTTTTGCTCACATGTCCTGCAGGCAGCTGCGCGCTCGCTCGCTCACTGAGGCCG
CCCGGGCAAAGCCCGGGCGTCGGGCGACCTTTGGTCGCCCGGCCTCAGTGAGCGAGCGAGCG
CGCAGAGAGGGAGTGGCCAACTCCATCACTAGGGGTTCCTGCGGCCGCACGCGTTTAATTAA
GACCTCGAAGGGGACTTGGGGGGTTCGGGGCTTTCGGGGGCGGTCGGGGGTTCGCGGACCCG
GGAAGCTCTGAGGACCCAGAGGCCGGGCGCGCTCCGCCCGCGGCGCCGCCCCCTCCGTAACT
TTCCCAGTCTCCGAGGGAAGAGGCGGGGTGTGGGGTGCGGTTAAAAGGCGCCACGGCGGGAG
ACAGGTGTTGCGGCCCCGCAGCGCCCGCGCGCTCCTCTCCCCGACTCGGAGCCCCTCGGCGG
CGCCCGGCCCAGGACCCGCCTAGGAGCGCAGGAGCCCCAGCGCAGAGACCCCAACGCCGAGA
CCCCCGCCCCGGCCCCGCCGCGCTTCCTCCCGACGCAGAGCAAACCGCCCAGAGTAGAAGCG
GATCCGCCACCATGGATTGGGGCACGCTGCAGACGATCCTGGGGGGTGTGAACAAACACTCC
ACCAGCATTGGAAAGATCTGGCTCACCGTCCTCTTCATTTTTCGCATTATGATCCTCGTTGT
GGCTGCAAAGGAGGTGTGGGGAGATGAGCAGGCCGACTTTGTCTGCAACACCCTGCAGCCAG
GCTGCAAGAACGTGTGCTACGATCACTACTTCCCCATCTCCCACATCCGGCTATGGGCCCTG
CAGCTGATCTTCGTGTCCACGCCAGCGCTCCTAGTGGCCATGCACGTGGCCTACCGGAGACA
TGAGAAGAAGAGGAAGTTCATCAAGGGGGAGATAAAGAGTGAATTTAAGGACATCGAGGAGA
TCAAAACCCAGAAGGTCCGCATCGAAGGCTCCCTGTGGTGGACCTACACAAGCAGCATCTTC
TTCCGGGTCATCTTCGAAGCCGCCTTCATGTACGTCTTCTATGTCATGTACGACGGCTTCTC
CATGCAGCGGCTGGTGAAGTGCAACGCCTGGCCTTGTCCCAACACTGTGGACTGCTTTGTGT
CCCGGCCCACGGAGAAGACTGTCTTCACAGTGTTCATGATTGCAGTGTCTGGAATTTGCATC
CTGCTGAATGTCACTGAATTGTGTTATTTGCTAATTAGATATTGTTCTGGGAAGTCAAAAAA
GCCAGTTTACCCATACGATGTTCCAGATTACGCTTAAGGCGCGCCACCCCTGCAGGGAATTC
CGCATTGCCCAGTTGTTAGATTAAGAAATAGACAGCATGAGAGGGATGAGGCAACCCGTGCT
CAGCTGTCAAGGCTCAGTCGCTAGCATTTCCCAACACAAAGATTCTGACCTTAAATGCAACC
ATTTGAAACCCCTGTAGGCCTCAGGTGAAACTCCAGATGCCACAATGGAGCTCTGCTCCCCT
AAAGCCTCAAAACAAAGGCCTAATTCTATGCCTGTCTTAATTTTCTTTCACTTAAGTTAGTT
CCACTGAGACCCCAGGCTGTTAGGGGTTATTGGTGTAAGGTACTTTCATATTTTAAACAGAG
GATATCGGCATTTGTTTCTTTCTCTGAGGACAAGAGAAAAAAGCCAGGTTCCACAGAGGACA
CAGAGAAGGTTTGGGTGTCCTCCTGGGGTTCTTTTTGCCAACTTTCCCCACGTTAAAGGTGA
ACATTGGTTCTTTCATTTGCTTTGGAAGTTTTAATCTCTAACAGTGGACAAAGTTACCAGTG
CCTTAAACTCTGTTACACTTTTTGGAAGTGAAAACTTTGTAGTATGATAGGTTATTTTGATG
TAAAGATGTTCTGGATACCATTATATGTTCCCCCTGTTTCAGAGGCTCAGATTGTAATATGT
AAATGGTATGTCATTCGCTACTATGATTTAATTTGAAATATGGTCTTTTGGTTATGAATACT
TTGCAGCACAGCTGAGAGGCTGTCTGTTGTATTCATTGTGGTCATAGCACCTAACAACATTG
TAGCCTCAATCGAGTGAGACAGACTAGAAGTTCCTAGTGATGGCTTATGATAGCAAATGGCC
TCATGTCAAATATTTAGATGTAATTTTGTGTAAGAAATACAGACTGGATGTACCACCAACTA
CTACCTGTAATGACAGGCCTGTCCAACACATCTCCCTTTTCCATGACTGTGGTAGCCAGCAT
CGGAAAGAACGCTGATTTAAAGAGGTCGCTTGGGAATTTTATTGACACAGTACCATTTAATG
GGGAGGACAAAATGGGGCAGGGGAGGGAGAAGTTTCTGTCGTTAAAAACAGATTTGGAAAGA
CTGGACTCTAAAGTCTGTTGATTAAAGATGAGCTTTGTCTACTTCAAAAGTTTGTTTGCTTA
CCCCTTCAGCCTCCAATTTTTTAAGTGAAAATATAGCTAATAACATGTGAAAAGAATAGAAG
CTAAGGTTTAGATAAATATTGAGCAGATCTATAGGAAGATTGAACCTGAATATTGCCATTAT
GCTTGACATGGTTTCCAAAAAATGGTACTCCACATATTTCAGTGAGGGTAAGTATTTTCCTG
TTGTCAAGAATAGCATTGTAAAAGCATTTTGTAATAATAAAGAATAGCTTTAATGATATGCT
TGTAACTAAAATAATTTTGTAATGTATCAAATACATTTAAAACATTAAAATATAATCTCTAT
AATAATTTAAAATCTAATATGGTTTTAATAGAACAGCGATATCAAGCTTATCGATAATCAAC
CTCTGGATTACAAAATTTGTGAAAGATTGACTGGTATTCTTAACTATGTTGCTCCTTTTACG
CTATGTGGATACGCTGCTTTAATGCCTTTGTATCATGCTATTGCTTCCCGTATGGCTTTCAT
TTTCTCCTCCTTGTATAAATCCTGGTTGCTGTCTCTTTATGAGGAGTTGTGGCCCGTTGTCA
GGCAACGTGGCGTGGTGTGCACTGTGTTTGCTGACGCAACCCCCACTGGTTGGGGCATTGCC
ACCACCTGTCAGCTCCTTTCCGGGACTTTCGCTTTCCCCCTCCCTATTGCCACGGCGGAACT
CATCGCCGCCTGCCTTGCCCGCTGCTGGACAGGGGCTCGGCTGTTGGGCACTGACAATTCCG
TGGTGTTGTCGGGGAAATCATCGTCCTTTCCTTGGCTGCTCGCCTATGTTGCCACCTGGATT
CTGCGCGGGACGTCCTTCTGCTACGTCCCTTCGGCCCTCAATCCAGCGGACCTTCCTTCCCG
CGGCCTGCTGCCGGCTCTGCGGCCTCTTCCGCGTCTTCGCCTTCGCCCTCAGACGAGTCGGA
TCTCCCTTTGGGCCGCCTCCCCGCGAATTCATCGATACCGAGCGCTGCTCGAGAGATCTGTG
ATAGCGGCCATCAAGCTGGCTGTGCCTTCTAGTTGCCAGCCATCTGTTGTTTGCCCCTCCCC
CGTGCCTTCCTTGACCCTGGAAGGTGCCACTCCCACTGTCCTTTCCTAATAAAATGAGGAAA
TTGCATCGCATTGTCTGAGTAGGTGTCATTCTATTCTGGGGGGTGGGGTGGGGCAGGACAGC
AAGGGGGAGGATTGGGAAGACAATAGCAGGCATGCTGGGGACACGTGCGGACCGAGCGGCCG
CAGGAACCCCTAGTGATGGAGTTGGCCACTCCCTCTCTGCGCGCTCGCTCGCTCACTGAGGC
CGGGCGACCAAAGGTCGCCCGACGCCCGGGCTTTGCCCGGGCGGCCTCAGTGAGCGAGCGAG
CGCGCAGCTGCCTGCAGGGGCGCCTGATGCGGTATTTTCTCCTTACGCATCTGTGCGGTATT
TCACACCGCATACGTCAAAGCAACCATAGTACGCGCCCTGTAGCGGCGCATTAAGCGCGGCG
GGTGTGGTGGTTACGCGCAGCGTGACCGCTACACTTGCCAGCGCCCTAGCGCCCGCTCCTTT
CGCTTTCTTCCCTTCCTTTCTCGCCACGTTCGCCGGCTTTCCCCGTCAAGCTCTAAATCGGG
GGCTCCCTTTAGGGTTCCGATTTAGTGCTTTACGGCACCTCGACCCCAAAAAACTTGATTTG
GGTGATGGTTCACGTAGTGGGCCATCGCCCTGATAGACGGTTTTTCGCCCTTTGACGTTGGA
GTCCACGTTCTTTAATAGTGGACTCTTGTTCCAAACTGGAACAACACTCAACCCTATCTCGG
GCTATTCTTTTGATTTATAAGGGATTTTGCCGATTTCGGCCTATTGGTTAAAAAATGAGCTG
ATTTAACAAAAATTTAACGCGAATTTTAACAAAATATTAACGTTTACAATTTTATGGTGCAC
TCTCAGTACAATCTGCTCTGATGCCGCATAGTTAAGCCAGCCCCGACACCCGCCAACACCCG
CTGACGCGCCCTGACGGGCTTGTCTGCTCCCGGCATCCGCTTACAGACAAGCTGTGACCGTC
TCCGGGAGCTGCATGTGTCAGAGGTTTTCACCGTCATCACCGAAACGCGCGAGACGAAAGGG
CCTCGTGATACGCCTATTTTTATAGGTTAATGTCATGATAATAATGGTTTCTTAGACGTCAG
GTGGCACTTTTCGGGGAAATGTGCGCGGAACCCCTATTTGTTTATTTTTCTAAATACATTCA
AATATGTATCCGCTCATGAGACAATAACCCTGATAAATGCTTCAATAATATTGAAAAAGGAA
GAGTATGAGTATTCAACATTTCCGTGTCGCCCTTATTCCCTTTTTTGCGGCATTTTGCCTTC
CTGTTTTTGCTCACCCAGAAACGCTGGTGAAAGTAAAAGATGCTGAAGATCAGTTGGGTGCA
CGAGTGGGTTACATCGAACTGGATCTCAACAGCGGTAAGATCCTTGAGAGTTTTCGCCCCGA
AGAACGTTTTCCAATGATGAGCACTTTTAAAGTTCTGCTATGTGGCGCGGTATTATCCCGTA
TTGACGCCGGGCAAGAGCAACTCGGTCGCCGCATACACTATTCTCAGAATGACTTGGTTGAG
TACTCACCAGTCACAGAAAAGCATCTTACGGATGGCATGACAGTAAGAGAATTATGCAGTGC
TGCCATAACCATGAGTGATAACACTGCGGCCAACTTACTTCTGACAACGATCGGAGGACCGA
AGGAGCTAACCGCTTTTTTGCACAACATGGGGGATCATGTAACTCGCCTTGATCGTTGGGAA
CCGGAGCTGAATGAAGCCATACCAAACGACGAGCGTGACACCACGATGCCTGTAGCAATGGC
AACAACGTTGCGCAAACTATTAACTGGCGAACTACTTACTCTAGCTTCCCGGCAACAATTAA
TAGACTGGATGGAGGCGGATAAAGTTGCAGGACCACTTCTGCGCTCGGCCCTTCCGGCTGGC
TGGTTTATTGCTGATAAATCTGGAGCCGGTGAGCGTGGGTCTCGCGGTATCATTGCAGCACT
GGGGCCAGATGGTAAGCCCTCCCGTATCGTAGTTATCTACACGACGGGGAGTCAGGCAACTA
TGGATGAACGAAATAGACAGATCGCTGAGATAGGTGCCTCACTGATTAAGCATTGGTAACTG
TCAGACCAAGTTTACTCATATATACTTTAGATTGATTTAAAACTTCATTTTTAATTTAAAAG
GATCTAGGTGAAGATCCTTTTTGATAATCTCATGACCAAAATCCCTTAACGTGAGTTTTCGT
TCCACTGAGCGTCAGACCCCGTAGAAAAGATCAAAGGATCTTCTTGAGATCCTTTTTTTCTG
CGCGTAATCTGCTGCTTGCAAACAAAAAAACCACCGCTACCAGCGGTGGTTTGTTTGCCGGA
TCAAGAGCTACCAACTCTTTTTCCGAAGGTAACTGGCTTCAGCAGAGCGCAGATACCAAATA
CTGTCCTTCTAGTGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAAGTCGTGTCTTAC
CGGGTTGGACTCAAGACGATAGTTACCGGATAAGGCGCAGCGGTCGGGCTGAACGGGGGGTT
CGTGCACACAGCCCAGCTTGGAGCGAACGACCTACACCGAACTGAGATACCTACAGCGTGAG
CTATGAGAAAGCGCCACGCTTCCCGAAGGGAGAAAGGCGGACAGGTATCCGGTAAGCGGCAG
GGTCGGAACAGGAGAGCGCACGAGGGAGCTTCCAGGGGGAAACGCCTGGTATCTTTATAGTC
CTGTCGGGTTTCGCCACCTCTGACTTGAGCGTCGATTTTTGTGATGCTCGTCAGGGGGGCGG
AGCCTATGGAAAAACGCCAGCAACGCGGCCTTTTTACGGTTCCTGGCCTT

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 62. An exemplary nucleotide sequence for vector c70 encoding a mouse GJB2 protein with an HA tag is set forth in SEQ ID NO: 62 (mouse GJB2 coding sequence in boldface; no HA tag):

CAGCACTGGGGCCAGATGGTAAGCCCTCCCGTATCGTAGTTATCTACACGACGGGGAGTCAG
GCAACTATGGATGAACGAAATAGACAGATCGCTGAGATAGGTGCCTCACTGATTAAGCATTG
GTAACTGTCAGACCAAGTTTACTCATATATACTTTAGATTGATTTAAAACTTCATTTTTAAT
TTAAAAGGATCTAGGTGAAGATCCTTTTTGATAATCTCATGACCAAAATCCCTTAACGTGAG
TTTTCGTTCCACTGAGCGTCAGACCCCGTAGAAAAGATCAAAGGATCTTCTTGAGATCCTTT
TTTTCTGCGCGTAATCTGCTGCTTGCAAACAAAAAAACCACCGCTACCAGCGGTGGTTTGTT
TGCCGGATCAAGAGCTACCAACTCTTTTTCCGAAGGTAACTGGCTTCAGCAGAGCGCAGATA
CCAAATACTGTCCTTCTAGTGTAGCCGTAGTTAGGCCACCACTTCAAGAACTCTGTAGCACC
GCCTACATACCTCGCTCTGCTAATCCTGTTACCAGTGGCTGCTGCCAGTGGCGATAAGTCGT
GTCTTACCGGGTTGGACTCAAGACGATAGTTACCGGATAAGGCGCAGCGGTCGGGCTGAACG
GGGGGTTCGTGCACACAGCCCAGCTTGGAGCGAACGACCTACACCGAACTGAGATACCTACA
GCGTGAGCTATGAGAAAGCGCCACGCTTCCCGAAGGGAGAAAGGCGGACAGGTATCCGGTAA
GCGGCAGGGTCGGAACAGGAGAGCGCACGAGGGAGCTTCCAGGGGGAAACGCCTGGTATCTT
TATAGTCCTGTCGGGTTTCGCCACCTCTGACTTGAGCGTCGATTTTTGTGATGCTCGTCAGG
GGGGCGGAGCCTATGGAAAAACGCCAGCAACGCGGCCTTTTTACGGTTCCTGGCCTTTTGCT
GGCCTTTTGCTCACATGTCCTGCAGGCAGCTGCGCGCTCGCTCGCTCACTGAGGCCGCCCGG
GCAAAGCCCGGGCGTCGGGCGACCTTTGGTCGCCCGGCCTCAGTGAGCGAGCGAGCGCGCAG
AGAGGGAGTGGCCAACTCCATCACTAGGGGTTCCTGCGGCCGCACGCGTTTAATTAAGACCT
CGAAGGGGACTTGGGGGGTTCGGGGCTTTCGGGGGCGGTCGGGGGTTCGCGGACCCGGGAAG
CTCTGAGGACCCAGAGGCCGGGCGCGCTCCGCCCGCGGCGCCGCCCCCTCCGTAACTTTCCC
AGTCTCCGAGGGAAGAGGCGGGGTGTGGGGTGCGGTTAAAAGGCGCCACGGCGGGAGACAGG
TGTTGCGGCCCCGCAGCGCCCGCGCGCTCCTCTCCCCGACTCGGAGCCCCTCGGCGGCGCCC
GGCCCAGGACCCGCCTAGGAGCGCAGGAGCCCCAGCGCAGAGACCCCAACGCCGAGACCCCC
GCCCCGGCCCCGCCGCGCTTCCTCCCGACGCAGAGCAAACCGCCCAGAGTAGAAGCGGATCC
GCCACCATGGATTGGGGCACACTCCAGAGCATCCTCGGGGGTGTCAACAAACACTCCACCAG
CATTGGAAAGATCTGGCTCACGGTCCTCTTCATCTTCCGCATCATGATCCTCGTGGTGGCTG
CAAAGGAGGTGTGGGGAGATGAGCAAGCCGATTTTGTCTGCAACACGCTCCAGCCTGGCTGC
AAGAATGTATGCTACGACCACCACTTCCCCATCTCTCACATCCGGCTCTGGGCTCTGCAGCT
GATCATGGTGTCCACGCCAGCCCTCCTGGTAGCTATGCATGTGGCCTACCGGAGACATGAAA
AGAAACGGAAGTTCATGAAGGGAGAGATAAAGAACGAGTTTAAGGACATCGAAGAGATCAAA
ACCCAGAAGGTCCGTATCGAAGGGTCCCTGTGGTGGACCTACACCACCAGCATCTTCTTCCG
GGTCATCTTTGAAGCCGTCTTCATGTACGTCTTTTACATCATGTACAATGGCTTCTTCATGC
AACGTCTGGTGAAATGCAACGCTTGGCCCTGCCCCAATACAGTGGACTGCTTCATTTCCAGG
CCCACAGAAAAGACTGTCTTCACCGTGTTTATGATTTCTGTGTCTGGAATTTGCATTCTGCT
AAATATCACAGAGCTGTGCTATTTGTTCGTTAGGTATTGCTCAGGAAAGTCCAAAAGACCAG
TCTAAGGCGCGCCACCCCTGCAGGGAATTCCGCATTGCCCAGTTGTTAGATTAAGAAATAGA
CAGCATGAGAGGGATGAGGCAACCCGTGCTCAGCTGTCAAGGCTCAGTCGCTAGCATTTCCC
AACACAAAGATTCTGACCTTAAATGCAACCATTTGAAACCCCTGTAGGCCTCAGGTGAAACT
CCAGATGCCACAATGGAGCTCTGCTCCCCTAAAGCCTCAAAACAAAGGCCTAATTCTATGCC
TGTCTTAATTTTCTTTCACTTAAGTTAGTTCCACTGAGACCCCAGGCTGTTAGGGGTTATTG
GTGTAAGGTACTTTCATATTTTAAACAGAGGATATCGGCATTTGTTTCTTTCTCTGAGGACA
AGAGAAAAAAGCCAGGTTCCACAGAGGACACAGAGAAGGTTTGGGTGTCCTCCTGGGGTTCT
TTTTGCCAACTTTCCCCACGTTAAAGGTGAACATTGGTTCTTTCATTTGCTTTGGAAGTTTT
AATCTCTAACAGTGGACAAAGTTACCAGTGCCTTAAACTCTGTTACACTTTTTGGAAGTGAA
AACTTTGTAGTATGATAGGTTATTTTGATGTAAAGATGTTCTGGATACCATTATATGTTCCC
CCTGTTTCAGAGGCTCAGATTGTAATATGTAAATGGTATGTCATTCGCTACTATGATTTAAT
TTGAAATATGGTCTTTTGGTTATGAATACTTTGCAGCACAGCTGAGAGGCTGTCTGTTGTAT
TCATTGTGGTCATAGCACCTAACAACATTGTAGCCTCAATCGAGTGAGACAGACTAGAAGTT
CCTAGTGATGGCTTATGATAGCAAATGGCCTCATGTCAAATATTTAGATGTAATTTTGTGTA
AGAAATACAGACTGGATGTACCACCAACTACTACCTGTAATGACAGGCCTGTCCAACACATC
TCCCTTTTCCATGACTGTGGTAGCCAGCATCGGAAAGAACGCTGATTTAAAGAGGTCGCTTG
GGAATTTTATTGACACAGTACCATTTAATGGGGAGGACAAAATGGGGCAGGGGAGGGAGAAG
TTTCTGTCGTTAAAAACAGATTTGGAAAGACTGGACTCTAAAGTCTGTTGATTAAAGATGAG
CTTTGTCTACTTCAAAAGTTTGTTTGCTTACCCCTTCAGCCTCCAATTTTTTAAGTGAAAAT
ATAGCTAATAACATGTGAAAAGAATAGAAGCTAAGGTTTAGATAAATATTGAGCAGATCTAT
AGGAAGATTGAACCTGAATATTGCCATTATGCTTGACATGGTTTCCAAAAAATGGTACTCCA
CATATTTCAGTGAGGGTAAGTATTTTCCTGTTGTCAAGAATAGCATTGTAAAAGCATTTTGT
AATAATAAAGAATAGCTTTAATGATATGCTTGTAACTAAAATAATTTTGTAATGTATCAAAT
ACATTTAAAACATTAAAATATAATCTCTATAATAATTTAAAATCTAATATGGTTTTAATAGA
ACAGCGATATCAAGCTTATCGATAATCAACCTCTGGATTACAAAATTTGTGAAAGATTGACT
GGTATTCTTAACTATGTTGCTCCTTTTACGCTATGTGGATACGCTGCTTTAATGCCTTTGTA
TCATGCTATTGCTTCCCGTATGGCTTTCATTTTCTCCTCCTTGTATAAATCCTGGTTGCTGT
CTCTTTATGAGGAGTTGTGGCCCGTTGTCAGGCAACGTGGCGTGGTGTGCACTGTGTTTGCT
GACGCAACCCCCACTGGTTGGGGCATTGCCACCACCTGTCAGCTCCTTTCCGGGACTTTCGC
TTTCCCCCTCCCTATTGCCACGGCGGAACTCATCGCCGCCTGCCTTGCCCGCTGCTGGACAG
GGGCTCGGCTGTTGGGCACTGACAATTCCGTGGTGTTGTCGGGGAAATCATCGTCCTTTCCT
TGGCTGCTCGCCTATGTTGCCACCTGGATTCTGCGCGGGACGTCCTTCTGCTACGTCCCTTC
GGCCCTCAATCCAGCGGACCTTCCTTCCCGCGGCCTGCTGCCGGCTCTGCGGCCTCTTCCGC
GTCTTCGCCTTCGCCCTCAGACGAGTCGGATCTCCCTTTGGGCCGCCTCCCCGCGAATTCAT
CGATACCGAGCGCTGCTCGAGAGATCTGTGATAGCGGCCATCAAGCTGGCTGTGCCTTCTAG
TTGCCAGCCATCTGTTGTTTGCCCCTCCCCCGTGCCTTCCTTGACCCTGGAAGGTGCCACTC
CCACTGTCCTTTCCTAATAAAATGAGGAAATTGCATCGCATTGTCTGAGTAGGTGTCATTCT
ATTCTGGGGGGTGGGGTGGGGCAGGACAGCAAGGGGGAGGATTGGGAAGACAATAGCAGGCA
TGCTGGGGACACGTGCGGACCGAGCGGCCGCAGGAACCCCTAGTGATGGAGTTGGCCACTCC
CTCTCTGCGCGCTCGCTCGCTCACTGAGGCCGGGCGACCAAAGGTCGCCCGACGCCCGGGCT
TTGCCCGGGCGGCCTCAGTGAGCGAGCGAGCGCGCAGCTGCCTGCAGGGGCGCCTGATGCGG
TATTTTCTCCTTACGCATCTGTGCGGTATTTCACACCGCATACGTCAAAGCAACCATAGTAC
GCGCCCTGTAGCGGCGCATTAAGCGCGGCGGGTGTGGTGGTTACGCGCAGCGTGACCGCTAC
ACTTGCCAGCGCCCTAGCGCCCGCTCCTTTCGCTTTCTTCCCTTCCTTTCTCGCCACGTTCG
CCGGCTTTCCCCGTCAAGCTCTAAATCGGGGGCTCCCTTTAGGGTTCCGATTTAGTGCTTTA
CGGCACCTCGACCCCAAAAAACTTGATTTGGGTGATGGTTCACGTAGTGGGCCATCGCCCTG
ATAGACGGTTTTTCGCCCTTTGACGTTGGAGTCCACGTTCTTTAATAGTGGACTCTTGTTCC
AAACTGGAACAACACTCAACCCTATCTCGGGCTATTCTTTTGATTTATAAGGGATTTTGCCG
ATTTCGGCCTATTGGTTAAAAAATGAGCTGATTTAACAAAAATTTAACGCGAATTTTAACAA
AATATTAACGTTTACAATTTTATGGTGCACTCTCAGTACAATCTGCTCTGATGCCGCATAGT
TAAGCCAGCCCCGACACCCGCCAACACCCGCTGACGCGCCCTGACGGGCTTGTCTGCTCCCG
GCATCCGCTTACAGACAAGCTGTGACCGTCTCCGGGAGCTGCATGTGTCAGAGGTTTTCACC
GTCATCACCGAAACGCGCGAGACGAAAGGGCCTCGTGATACGCCTATTTTTATAGGTTAATG
TCATGATAATAATGGTTTCTTAGACGTCAGGTGGCACTTTTCGGGGAAATGTGCGCGGAACC
CCTATTTGTTTATTTTTCTAAATACATTCAAATATGTATCCGCTCATGAGACAATAACCCTG
ATAAATGCTTCAATAATATTGAAAAAGGAAGAGTATGAGTATTCAACATTTCCGTGTCGCCC
TTATTCCCTTTTTTGCGGCATTTTGCCTTCCTGTTTTTGCTCACCCAGAAACGCTGGTGAAA
GTAAAAGATGCTGAAGATCAGTTGGGTGCACGAGTGGGTTACATCGAACTGGATCTCAACAG
CGGTAAGATCCTTGAGAGTTTTCGCCCCGAAGAACGTTTTCCAATGATGAGCACTTTTAAAG
TTCTGCTATGTGGCGCGGTATTATCCCGTATTGACGCCGGGCAAGAGCAACTCGGTCGCCGC
ATACACTATTCTCAGAATGACTTGGTTGAGTACTCACCAGTCACAGAAAAGCATCTTACGGA
TGGCATGACAGTAAGAGAATTATGCAGTGCTGCCATAACCATGAGTGATAACACTGCGGCCA
ACTTACTTCTGACAACGATCGGAGGACCGAAGGAGCTAACCGCTTTTTTGCACAACATGGGG
GATCATGTAACTCGCCTTGATCGTTGGGAACCGGAGCTGAATGAAGCCATACCAAACGACGA
GCGTGACACCACGATGCCTGTAGCAATGGCAACAACGTTGCGCAAACTATTAACTGGCGAAC
TACTTACTCTAGCTTCCCGGCAACAATTAATAGACTGGATGGAGGCGGATAAAGTTGCAGGA
CCACTTCTGCGCTCGGCCCTTCCGGCTGGCTGGTTTATTGCTGATAAATCTGGAGCCGGTGA
GCGTGGGTCTCGCGGTATCATTG

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 63. An exemplary nucleotide sequence for vector c70 encoding a mouse GJB2 protein with a HA tag is set forth in SEQ ID NO: 63 (human GJB2 coding sequence in boldface; no HA tag):

CAGCACTGGGGCCAGATGGTAAGCCCTCCCGTATCGTAGTTATCTACACGACGGGGAGTCAG
GCAACTATGGATGAACGAAATAGACAGATCGCTGAGATAGGTGCCTCACTGATTAAGCATTG
GTAACTGTCAGACCAAGTTTACTCATATATACTTTAGATTGATTTAAAACTTCATTTTTAAT
TTAAAAGGATCTAGGTGAAGATCCTTTTTGATAATCTCATGACCAAAATCCCTTAACGTGAG
TTTTCGTTCCACTGAGCGTCAGACCCCGTAGAAAAGATCAAAGGATCTTCTTGAGATCCTTT
TTTTCTGCGCGTAATCTGCTGCTTGCAAACAAAAAAACCACCGCTACCAGCGGTGGTTTGTT
TGCCGGATCAAGAGCTACCAACTCTTTTTCCGAAGGTAACTGGCTTCAGCAGAGCGCAGATA
CCAAATACTGTCCTTCTAGTGTAGCCGTAGTTAGGCCACCACTTCAAGAACTCTGTAGCACC
GCCTACATACCTCGCTCTGCTAATCCTGTTACCAGTGGCTGCTGCCAGTGGCGATAAGTCGT
GTCTTACCGGGTTGGACTCAAGACGATAGTTACCGGATAAGGCGCAGCGGTCGGGCTGAACG
GGGGGTTCGTGCACACAGCCCAGCTTGGAGCGAACGACCTACACCGAACTGAGATACCTACA
GCGTGAGCTATGAGAAAGCGCCACGCTTCCCGAAGGGAGAAAGGCGGACAGGTATCCGGTAA
GCGGCAGGGTCGGAACAGGAGAGCGCACGAGGGAGCTTCCAGGGGGAAACGCCTGGTATCTT
TATAGTCCTGTCGGGTTTCGCCACCTCTGACTTGAGCGTCGATTTTTGTGATGCTCGTCAGG
GGGGCGGAGCCTATGGAAAAACGCCAGCAACGCGGCCTTTTTACGGTTCCTGGCCTTTTGCT
GGCCTTTTGCTCACATGTCCTGCAGGCAGCTGCGCGCTCGCTCGCTCACTGAGGCCGCCCGG
GCAAAGCCCGGGCGTCGGGCGACCTTTGGTCGCCCGGCCTCAGTGAGCGAGCGAGCGCGCAG
AGAGGGAGTGGCCAACTCCATCACTAGGGGTTCCTGCGGCCGCACGCGTTTAATTAAGACCT
CGAAGGGGACTTGGGGGGTTCGGGGCTTTCGGGGGCGGTCGGGGGTTCGCGGACCCGGGAAG
CTCTGAGGACCCAGAGGCCGGGCGCGCTCCGCCCGCGGCGCCGCCCCCTCCGTAACTTTCCC
AGTCTCCGAGGGAAGAGGCGGGGTGTGGGGTGCGGTTAAAAGGCGCCACGGCGGGAGACAGG
TGTTGCGGCCCCGCAGCGCCCGCGCGCTCCTCTCCCCGACTCGGAGCCCCTCGGCGGCGCCC
GGCCCAGGACCCGCCTAGGAGCGCAGGAGCCCCAGCGCAGAGACCCCAACGCCGAGACCCCC
GCCCCGGCCCCGCCGCGCTTCCTCCCGACGCAGAGCAAACCGCCCAGAGTAGAAGCGGATCC
GCCACCATGGATTGGGGCACGCTGCAGACGATCCTGGGGGGTGTGAACAAACACTCCACCAG
CATTGGAAAGATCTGGCTCACCGTCCTCTTCATTTTTCGCATTATGATCCTCGTTGTGGCTG
CAAAGGAGGTGTGGGGAGATGAGCAGGCCGACTTTGTCTGCAACACCCTGCAGCCAGGCTGC
AAGAACGTGTGCTACGATCACTACTTCCCCATCTCCCACATCCGGCTATGGGCCCTGCAGCT
GATCTTCGTGTCCACGCCAGCGCTCCTAGTGGCCATGCACGTGGCCTACCGGAGACATGAGA
AGAAGAGGAAGTTCATCAAGGGGGAGATAAAGAGTGAATTTAAGGACATCGAGGAGATCAAA
ACCCAGAAGGTCCGCATCGAAGGCTCCCTGTGGTGGACCTACACAAGCAGCATCTTCTTCCG
GGTCATCTTCGAAGCCGCCTTCATGTACGTCTTCTATGTCATGTACGACGGCTTCTCCATGC
AGCGGCTGGTGAAGTGCAACGCCTGGCCTTGTCCCAACACTGTGGACTGCTTTGTGTCCCGG
CCCACGGAGAAGACTGTCTTCACAGTGTTCATGATTGCAGTGTCTGGAATTTGCATCCTGCT
GAATGTCACTGAATTGTGTTATTTGCTAATTAGATATTGTTCTGGGAAGTCAAAAAAGCCAG
TTTAAGGCGCGCCACCCCTGCAGGGAATTCCGCATTGCCCAGTTGTTAGATTAAGAAATAGA
CAGCATGAGAGGGATGAGGCAACCCGTGCTCAGCTGTCAAGGCTCAGTCGCTAGCATTTCCC
AACACAAAGATTCTGACCTTAAATGCAACCATTTGAAACCCCTGTAGGCCTCAGGTGAAACT
CCAGATGCCACAATGGAGCTCTGCTCCCCTAAAGCCTCAAAACAAAGGCCTAATTCTATGCC
TGTCTTAATTTTCTTTCACTTAAGTTAGTTCCACTGAGACCCCAGGCTGTTAGGGGTTATTG
GTGTAAGGTACTTTCATATTTTAAACAGAGGATATCGGCATTTGTTTCTTTCTCTGAGGACA
AGAGAAAAAAGCCAGGTTCCACAGAGGACACAGAGAAGGTTTGGGTGTCCTCCTGGGGTTCT
TTTTGCCAACTTTCCCCACGTTAAAGGTGAACATTGGTTCTTTCATTTGCTTTGGAAGTTTT
AATCTCTAACAGTGGACAAAGTTACCAGTGCCTTAAACTCTGTTACACTTTTTGGAAGTGAA
AACTTTGTAGTATGATAGGTTATTTTGATGTAAAGATGTTCTGGATACCATTATATGTTCCC
CCTGTTTCAGAGGCTCAGATTGTAATATGTAAATGGTATGTCATTCGCTACTATGATTTAAT
TTGAAATATGGTCTTTTGGTTATGAATACTTTGCAGCACAGCTGAGAGGCTGTCTGTTGTAT
TCATTGTGGTCATAGCACCTAACAACATTGTAGCCTCAATCGAGTGAGACAGACTAGAAGTT
CCTAGTGATGGCTTATGATAGCAAATGGCCTCATGTCAAATATTTAGATGTAATTTTGTGTA
AGAAATACAGACTGGATGTACCACCAACTACTACCTGTAATGACAGGCCTGTCCAACACATC
TCCCTTTTCCATGACTGTGGTAGCCAGCATCGGAAAGAACGCTGATTTAAAGAGGTCGCTTG
GGAATTTTATTGACACAGTACCATTTAATGGGGAGGACAAAATGGGGCAGGGGAGGGAGAAG
TTTCTGTCGTTAAAAACAGATTTGGAAAGACTGGACTCTAAAGTCTGTTGATTAAAGATGAG
CTTTGTCTACTTCAAAAGTTTGTTTGCTTACCCCTTCAGCCTCCAATTTTTTAAGTGAAAAT
ATAGCTAATAACATGTGAAAAGAATAGAAGCTAAGGTTTAGATAAATATTGAGCAGATCTAT
AGGAAGATTGAACCTGAATATTGCCATTATGCTTGACATGGTTTCCAAAAAATGGTACTCCA
CATATTTCAGTGAGGGTAAGTATTTTCCTGTTGTCAAGAATAGCATTGTAAAAGCATTTTGT
AATAATAAAGAATAGCTTTAATGATATGCTTGTAACTAAAATAATTTTGTAATGTATCAAAT
ACATTTAAAACATTAAAATATAATCTCTATAATAATTTAAAATCTAATATGGTTTTAATAGA
ACAGCGATATCAAGCTTATCGATAATCAACCTCTGGATTACAAAATTTGTGAAAGATTGACT
GGTATTCTTAACTATGTTGCTCCTTTTACGCTATGTGGATACGCTGCTTTAATGCCTTTGTA
TCATGCTATTGCTTCCCGTATGGCTTTCATTTTCTCCTCCTTGTATAAATCCTGGTTGCTGT
CTCTTTATGAGGAGTTGTGGCCCGTTGTCAGGCAACGTGGCGTGGTGTGCACTGTGTTTGCT
GACGCAACCCCCACTGGTTGGGGCATTGCCACCACCTGTCAGCTCCTTTCCGGGACTTTCGC
TTTCCCCCTCCCTATTGCCACGGCGGAACTCATCGCCGCCTGCCTTGCCCGCTGCTGGACAG
GGGCTCGGCTGTTGGGCACTGACAATTCCGTGGTGTTGTCGGGGAAATCATCGTCCTTTCCT
TGGCTGCTCGCCTATGTTGCCACCTGGATTCTGCGCGGGACGTCCTTCTGCTACGTCCCTTC
GGCCCTCAATCCAGCGGACCTTCCTTCCCGCGGCCTGCTGCCGGCTCTGCGGCCTCTTCCGC
GTCTTCGCCTTCGCCCTCAGACGAGTCGGATCTCCCTTTGGGCCGCCTCCCCGCGAATTCAT
CGATACCGAGCGCTGCTCGAGAGATCTGTGATAGCGGCCATCAAGCTGGCTGTGCCTTCTAG
TTGCCAGCCATCTGTTGTTTGCCCCTCCCCCGTGCCTTCCTTGACCCTGGAAGGTGCCACTC
CCACTGTCCTTTCCTAATAAAATGAGGAAATTGCATCGCATTGTCTGAGTAGGTGTCATTCT
ATTCTGGGGGGTGGGGTGGGGCAGGACAGCAAGGGGGAGGATTGGGAAGACAATAGCAGGCA
TGCTGGGGACACGTGCGGACCGAGCGGCCGCAGGAACCCCTAGTGATGGAGTTGGCCACTCC
CTCTCTGCGCGCTCGCTCGCTCACTGAGGCCGGGCGACCAAAGGTCGCCCGACGCCCGGGCT
TTGCCCGGGCGGCCTCAGTGAGCGAGCGAGCGCGCAGCTGCCTGCAGGGGCGCCTGATGCGG
TATTTTCTCCTTACGCATCTGTGCGGTATTTCACACCGCATACGTCAAAGCAACCATAGTAC
GCGCCCTGTAGCGGCGCATTAAGCGCGGCGGGTGTGGTGGTTACGCGCAGCGTGACCGCTAC
ACTTGCCAGCGCCCTAGCGCCCGCTCCTTTCGCTTTCTTCCCTTCCTTTCTCGCCACGTTCG
CCGGCTTTCCCCGTCAAGCTCTAAATCGGGGGCTCCCTTTAGGGTTCCGATTTAGTGCTTTA
CGGCACCTCGACCCCAAAAAACTTGATTTGGGTGATGGTTCACGTAGTGGGCCATCGCCCTG
ATAGACGGTTTTTCGCCCTTTGACGTTGGAGTCCACGTTCTTTAATAGTGGACTCTTGTTCC
AAACTGGAACAACACTCAACCCTATCTCGGGCTATTCTTTTGATTTATAAGGGATTTTGCCG
ATTTCGGCCTATTGGTTAAAAAATGAGCTGATTTAACAAAAATTTAACGCGAATTTTAACAA
AATATTAACGTTTACAATTTTATGGTGCACTCTCAGTACAATCTGCTCTGATGCCGCATAGT
TAAGCCAGCCCCGACACCCGCCAACACCCGCTGACGCGCCCTGACGGGCTTGTCTGCTCCCG
GCATCCGCTTACAGACAAGCTGTGACCGTCTCCGGGAGCTGCATGTGTCAGAGGTTTTCACC
GTCATCACCGAAACGCGCGAGACGAAAGGGCCTCGTGATACGCCTATTTTTATAGGTTAATG
TCATGATAATAATGGTTTCTTAGACGTCAGGTGGCACTTTTCGGGGAAATGTGCGCGGAACC
CCTATTTGTTTATTTTTCTAAATACATTCAAATATGTATCCGCTCATGAGACAATAACCCTG
ATAAATGCTTCAATAATATTGAAAAAGGAAGAGTATGAGTATTCAACATTTCCGTGTCGCCC
TTATTCCCTTTTTTGCGGCATTTTGCCTTCCTGTTTTTGCTCACCCAGAAACGCTGGTGAAA
GTAAAAGATGCTGAAGATCAGTTGGGTGCACGAGTGGGTTACATCGAACTGGATCTCAACAG
CGGTAAGATCCTTGAGAGTTTTCGCCCCGAAGAACGTTTTCCAATGATGAGCACTTTTAAAG
TTCTGCTATGTGGCGCGGTATTATCCCGTATTGACGCCGGGCAAGAGCAACTCGGTCGCCGC
ATACACTATTCTCAGAATGACTTGGTTGAGTACTCACCAGTCACAGAAAAGCATCTTACGGA
TGGCATGACAGTAAGAGAATTATGCAGTGCTGCCATAACCATGAGTGATAACACTGCGGCCA
ACTTACTTCTGACAACGATCGGAGGACCGAAGGAGCTAACCGCTTTTTTGCACAACATGGGG
GATCATGTAACTCGCCTTGATCGTTGGGAACCGGAGCTGAATGAAGCCATACCAAACGACGA
GCGTGACACCACGATGCCTGTAGCAATGGCAACAACGTTGCGCAAACTATTAACTGGCGAAC
TACTTACTCTAGCTTCCCGGCAACAATTAATAGACTGGATGGAGGCGGATAAAGTTGCAGGA
CCACTTCTGCGCTCGGCCCTTCCGGCTGGCTGGTTTATTGCTGATAAATCTGGAGCCGGTGA
GCGTGGGTCTCGCGGTATCATTG

In some embodiments, an AAV vector described herein comprises an AAV 5′ ITR, a GJB2 GRE enhancer (hGJB2 GRE1), a GJB2 basal promoter, a GJB2 exon 1 5′ UTR, Kozak sequence, nucleotide sequence encoding a gene product (e.g., GJB2 or GFP), a GJB2 exon 2 3′ UTR, a WPRE, a bovine growth hormone poly A signal, and an AAV 3′ ITR (e.g., vector c81.1).

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 64. An exemplary nucleotide sequence for vector c81.1 encoding eGFP is set forth in SEQ ID NO: 64 (hGJB2 GRE1 underlined; eGFP coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTA
AGAGAGCACTTGGGAAGAGCCCCCGAGGGCAGCCGGGGCTTGCCGCCTCACCCTTTTGGTTT
CACATCCCAGAAATCAGTAAGGCAGGAATTGGAGGCTGCTTCTTGCCTTAGCAACTCGGTGA
CCTTAGGCAGAACAGTTCAGCCTTCTGAGTGTCCTTCCTCTTCTGTAAGGGGAGCGTAAACC
GTCCTCCATGCAGAACGTGTACTGTGCCTGGCACAGCACTGGGGCATTAGGATCTCCAAATT
AAAGGCTCACTCTGCGGGATGGAGGCAGCCACAGCTGGAAGAAGGAACATTTGGGGCCAGAA
GTCCCCCTACCTCCGTCCTAAGAGAGAAGATGGGAATAACGACCCTCGCTGAAATGATTGCT
CTCTGGCCAGCTCGCCTCGCATCCACATCCAAATCTGGGAGGCACAGAGCGCATCAGGACAT
CGGGTTCTGTCAGTGTAATGGGCGTGGCTCCTGACCTTCTGTCTGTATCAGAGAAGATAAGG
GAGAACATTTGAAAGAAAGGAGAAAGAAGATAGCCACTGGAGAACAGAGCAAAGGAGCCAGC
AGAAAAAGACGAGACGGCTGTAGCCCCACAGGAAGCAGAAACCGATAGGCTAAGTAGGATAC
ACACAAAGAAAAGTAGATCCCGAGAGGCATTTCCCCGAGGGCTTTCATGTGGTTTCTCGTGA
GGAGAAGCTGACTGCAGGGTGTTTGAAAGAACGACTTATGCAGCCATAAAAAATGATGAGTT
CATGTCCTTTGTAGGGACATGGATGATTAATTAAGACCTCGAAGGGGACTTGGGGGGTTCGG
GGCTTTCGGGGGCGGTCGGGGGTTCGCGGACCCGGGAAGCTCTGAGGACCCAGAGGCCGGGC
GCGCTCCGCCCGCGGCGCCGCCCCCTCCGTAACTTTCCCAGTCTCCGAGGGAAGAGGCGGGG
TGTGGGGTGCGGTTAAAAGGCGCCACGGCGGGAGACAGGTGTTGCGGCCCCGCAGCGCCCGC
GCGCTCCTCTCCCCGACTCGGAGCCCCTCGGCGGCGCCCGGCCCAGGACCCGCCTAGGAGCG
CAGGAGCCCCAGCGCAGAGACCCCAACGCCGAGACCCCCGCCCCGGCCCCGCCGCGCTTCCT
CCCGACGCAGAGCAAACCGCCCAGAGTAGAAGCCATGGTGAGCAAGGGCGAGGAGCTGTTCA
CCGGGGTGGTGCCCATCCTGGTCGAGCTGGACGGCGACGTAAACGGCCACAAGTTCAGCGTG
TCCGGCGAGGGCGAGGGCGATGCCACCTACGGCAAGCTGACCCTGAAGTTCATCTGCACCAC
CGGCAAGCTGCCCGTGCCCTGGCCCACCCTCGTGACCACCCTGACCTACGGCGTGCAGTGCT
TCAGCCGCTACCCCGACCACATGAAGCAGCACGACTTCTTCAAGTCCGCCATGCCCGAAGGC
TACGTCCAGGAGCGCACCATCTTCTTCAAGGACGACGGCAACTACAAGACCCGCGCCGAGGT
GAAGTTCGAGGGCGACACCCTGGTGAACCGCATCGAGCTGAAGGGCATCGACTTCAAGGAGG
ACGGCAACATCCTGGGGCACAAGCTGGAGTACAACTACAACAGCCACAACGTCTATATCATG
GCCGACAAGCAGAAGAACGGCATCAAGGTGAACTTCAAGATCCGCCACAACATCGAGGACGG
CAGCGTGCAGCTCGCCGACCACTACCAGCAGAACACCCCCATCGGCGACGGCCCCGTGCTGC
TGCCCGACAACCACTACCTGAGCACCCAGTCCGCCCTGAGCAAAGACCCCAACGAGAAGCGC
GATCACATGGTCCTGCTGGAGTTCGTGACCGCCGCCGGGATCACTCTCGGCATGGACGAGCT
GTACAAGTAATAAAGGCGCGCCACCCCTGCAGGGAATTCCGCATTGCCCAGTTGTTAGATTA
AGAAATAGACAGCATGAGAGGGATGAGGCAACCCGTGCTCAGCTGTCAAGGCTCAGTCGCTA
GCATTTCCCAACACAAAGATTCTGACCTTAAATGCAACCATTTGAAACCCCTGTAGGCCTCA
GGTGAAACTCCAGATGCCACAATGGAGCTCTGCTCCCCTAAAGCCTCAAAACAAAGGCCTAA
TTCTATGCCTGTCTTAATTTTCTTTCACTTAAGTTAGTTCCACTGAGACCCCAGGCTGTTAG
GGGTTATTGGTGTAAGGTACTTTCATATTTTAAACAGAGGATATCGGCATTTGTTTCTTTCT
CTGAGGACAAGAGAAAAAAGCCAGGTTCCACAGAGGACACAGAGAAGGTTTGGGTGTCCTCC
TGGGGTTCTTTTTGCCAACTTTCCCCACGTTAAAGGTGAACATTGGTTCTTTCATTTGCTTT
GGAAGTTTTAATCTCTAACAGTGGACAAAGTTACCAGTGCCTTAAACTCTGTTACACTTTTT
GGAAGTGAAAACTTTGTAGTATGATAGGTTATTTTGATGTAAAGATGTTCTGGATACCATTA
TATGTTCCCCCTGTTTCAGAGGCTCAGATTGTAATATGTAAATGGTATGTCATTCGCTACTA
TGATTTAATTTGAAATATGGTCTTTTGGTTATGAATACTTTGCAGCACAGCTGAGAGGCTGT
CTGTTGTATTCATTGTGGTCATAGCACCTAACAACATTGTAGCCTCAATCGAGTGAGACAGA
CTAGAAGTTCCTAGTGATGGCTTATGATAGCAAATGGCCTCATGTCAAATATTTAGATGTAA
TTTTGTGTAAGAAATACAGACTGGATGTACCACCAACTACTACCTGTAATGACAGGCCTGTC
CAACACATCTCCCTTTTCCATGACTGTGGTAGCCAGCATCGGAAAGAACGCTGATTTAAAGA
GGTCGCTTGGGAATTTTATTGACACAGTACCATTTAATGGGGAGGACAAAATGGGGCAGGGG
AGGGAGAAGTTTCTGTCGTTAAAAACAGATTTGGAAAGACTGGACTCTAAAGTCTGTTGATT
AAAGATGAGCTTTGTCTACTTCAAAAGTTTGTTTGCTTACCCCTTCAGCCTCCAATTTTTTA
AGTGAAAATATAGCTAATAACATGTGAAAAGAATAGAAGCTAAGGTTTAGATAAATATTGAG
CAGATCTATAGGAAGATTGAACCTGAATATTGCCATTATGCTTGACATGGTTTCCAAAAAAT
GGTACTCCACATATTTCAGTGAGGGTAAGTATTTTCCTGTTGTCAAGAATAGCATTGTAAAA
GCATTTTGTAATAATAAAGAATAGCTTTAATGATATGCTTGTAACTAAAATAATTTTGTAAT
GTATCAAATACATTTAAAACATTAAAATATAATCTCTATAATAATTTAAAATCTAATATGGT
TTTAATAGAACAGCGATATCAAGCTTATCGATAATCAACCTCTGGATTACAAAATTTGTGAA
AGATTGACTGGTATTCTTAACTATGTTGCTCCTTTTACGCTATGTGGATACGCTGCTTTAAT
GCCTTTGTATCATGCTATTGCTTCCCGTATGGCTTTCATTTTCTCCTCCTTGTATAAATCCT
GGTTGCTGTCTCTTTATGAGGAGTTGTGGCCCGTTGTCAGGCAACGTGGCGTGGTGTGCACT
GTGTTTGCTGACGCAACCCCCACTGGTTGGGGCATTGCCACCACCTGTCAGCTCCTTTCCGG
GACTTTCGCTTTCCCCCTCCCTATTGCCACGGCGGAACTCATCGCCGCCTGCCTTGCCCGCT
GCTGGACAGGGGCTCGGCTGTTGGGCACTGACAATTCCGTGGTGTTGTCGGGGAAATCATCG
TCCTTTCCTTGGCTGCTCGCCTATGTTGCCACCTGGATTCTGCGCGGGACGTCCTTCTGCTA
CGTCCCTTCGGCCCTCAATCCAGCGGACCTTCCTTCCCGCGGCCTGCTGCCGGCTCTGCGGC
CTCTTCCGCGTCTTCGCCTTCGCCCTCAGACGAGTCGGATCTCCCTTTGGGCCGCCTCCCCG
CGAATTCATCGATACCGAGCGCTGCTCGAGAGATCTGTGATAGCGGCCATCAAGCTGGCTGT
GCCTTCTAGTTGCCAGCCATCTGTTGTTTGCCCCTCCCCCGTGCCTTCCTTGACCCTGGAAG
GTGCCACTCCCACTGTCCTTTCCTAATAAAATGAGGAAATTGCATCGCATTGTCTGAGTAGG
TGTCATTCTATTCTGGGGGGTGGGGTGGGGCAGGACAGCAAGGGGGAGGATTGGGAAGACAA
TAGCAGGCATGCTGGGGACACGTGCGGACCGAGCGGCCGCAGGAACCCCTAGTGATGGAGTT
GGCCACTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGCAGCTGCCTGCAGGGGCGC
CTGATGCGGTATTTTCTCCTTACGCATCTGTGCGGTATTTCACACCGCATACGTCAAAGCAA
CCATAGTACGCGCCCTGTAGCGGCGCATTAAGCGCGGCGGGTGTGGTGGTTACGCGCAGCGT
GACCGCTACACTTGCCAGCGCCCTAGCGCCCGCTCCTTTCGCTTTCTTCCCTTCCTTTCTCG
CCACGTTCGCCGGCTTTCCCCGTCAAGCTCTAAATCGGGGGCTCCCTTTAGGGTTCCGATTT
AGTGCTTTACGGCACCTCGACCCCAAAAAACTTGATTTGGGTGATGGTTCACGTAGTGGGCC
ATCGCCCTGATAGACGGTTTTTCGCCCTTTGACGTTGGAGTCCACGTTCTTTAATAGTGGAC
TCTTGTTCCAAACTGGAACAACACTCAACCCTATCTCGGGCTATTCTTTTGATTTATAAGGG
ATTTTGCCGATTTCGGCCTATTGGTTAAAAAATGAGCTGATTTAACAAAAATTTAACGCGAA
TTTTAACAAAATATTAACGTTTACAATTTTATGGTGCACTCTCAGTACAATCTGCTCTGATG
CCGCATAGTTAAGCCAGCCCCGACACCCGCCAACACCCGCTGACGCGCCCTGACGGGCTTGT
CTGCTCCCGGCATCCGCTTACAGACAAGCTGTGACCGTCTCCGGGAGCTGCATGTGTCAGAG
GTTTTCACCGTCATCACCGAAACGCGCGAGACGAAAGGGCCTCGTGATACGCCTATTTTTAT
AGGTTAATGTCATGATAATAATGGTTTCTTAGACGTCAGGTGGCACTTTTCGGGGAAATGTG
CGCGGAACCCCTATTTGTTTATTTTTCTAAATACATTCAAATATGTATCCGCTCATGAGACA
ATAACCCTGATAAATGCTTCAATAATATTGAAAAAGGAAGAGTATGAGTATTCAACATTTCC
GTGTCGCCCTTATTCCCTTTTTTGCGGCATTTTGCCTTCCTGTTTTTGCTCACCCAGAAACG
CTGGTGAAAGTAAAAGATGCTGAAGATCAGTTGGGTGCACGAGTGGGTTACATCGAACTGGA
TCTCAACAGCGGTAAGATCCTTGAGAGTTTTCGCCCCGAAGAACGTTTTCCAATGATGAGCA
CTTTTAAAGTTCTGCTATGTGGCGCGGTATTATCCCGTATTGACGCCGGGCAAGAGCAACTC
GGTCGCCGCATACACTATTCTCAGAATGACTTGGTTGAGTACTCACCAGTCACAGAAAAGCA
TCTTACGGATGGCATGACAGTAAGAGAATTATGCAGTGCTGCCATAACCATGAGTGATAACA
CTGCGGCCAACTTACTTCTGACAACGATCGGAGGACCGAAGGAGCTAACCGCTTTTTTGCAC
AACATGGGGGATCATGTAACTCGCCTTGATCGTTGGGAACCGGAGCTGAATGAAGCCATACC
AAACGACGAGCGTGACACCA

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 65. An exemplary nucleotide sequence for vector c81.1 encoding human GJB2 is set forth in SEQ ID NO: 65 (hGJB2 GRE1 underlined; human GJB2 coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTA
AGAGAGCACTTGGGAAGAGCCCCCGAGGGCAGCCGGGGCTTGCCGCCTCACCCTTTTGGTTT
CACATCCCAGAAATCAGTAAGGCAGGAATTGGAGGCTGCTTCTTGCCTTAGCAACTCGGTGA
CCTTAGGCAGAACAGTTCAGCCTTCTGAGTGTCCTTCCTCTTCTGTAAGGGGAGCGTAAACC
GTCCTCCATGCAGAACGTGTACTGTGCCTGGCACAGCACTGGGGCATTAGGATCTCCAAATT
AAAGGCTCACTCTGCGGGATGGAGGCAGCCACAGCTGGAAGAAGGAACATTTGGGGCCAGAA
GTCCCCCTACCTCCGTCCTAAGAGAGAAGATGGGAATAACGACCCTCGCTGAAATGATTGCT
CTCTGGCCAGCTCGCCTCGCATCCACATCCAAATCTGGGAGGCACAGAGCGCATCAGGACAT
CGGGTTCTGTCAGTGTAATGGGCGTGGCTCCTGACCTTCTGTCTGTATCAGAGAAGATAAGG
GAGAACATTTGAAAGAAAGGAGAAAGAAGATAGCCACTGGAGAACAGAGCAAAGGAGCCAGC
AGAAAAAGACGAGACGGCTGTAGCCCCACAGGAAGCAGAAACCGATAGGCTAAGTAGGATAC
ACACAAAGAAAAGTAGATCCCGAGAGGCATTTCCCCGAGGGCTTTCATGTGGTTTCTCGTGA
GGAGAAGCTGACTGCAGGGTGTTTGAAAGAACGACTTATGCAGCCATAAAAAATGATGAGTT
CATGTCCTTTGTAGGGACATGGATGATTAATTAAGACCTCGAAGGGGACTTGGGGGGTTCGG
GGCTTTCGGGGGCGGTCGGGGGTTCGCGGACCCGGGAAGCTCTGAGGACCCAGAGGCCGGGC
GCGCTCCGCCCGCGGCGCCGCCCCCTCCGTAACTTTCCCAGTCTCCGAGGGAAGAGGCGGGG
TGTGGGGTGCGGTTAAAAGGCGCCACGGCGGGAGACAGGTGTTGCGGCCCCGCAGCGCCCGC
GCGCTCCTCTCCCCGACTCGGAGCCCCTCGGCGGCGCCCGGCCCAGGACCCGCCTAGGAGCG
CAGGAGCCCCAGCGCAGAGACCCCAACGCCGAGACCCCCGCCCCGGCCCCGCCGCGCTTCCT
CCCGACGCAGAGCAAACCGCCCAGAGTAGAAGCCATGGATTGGGGCACGCTGCAGACGATCC
TGGGGGGTGTGAACAAACACTCCACCAGCATTGGAAAGATCTGGCTCACCGTCCTCTTCATT
TTTCGCATTATGATCCTCGTTGTGGCTGCAAAGGAGGTGTGGGGAGATGAGCAGGCCGACTT
TGTCTGCAACACCCTGCAGCCAGGCTGCAAGAACGTGTGCTACGATCACTACTTCCCCATCT
CCCACATCCGGCTATGGGCCCTGCAGCTGATCTTCGTGTCCACGCCAGCGCTCCTAGTGGCC
ATGCACGTGGCCTACCGGAGACATGAGAAGAAGAGGAAGTTCATCAAGGGGGAGATAAAGAG
TGAATTTAAGGACATCGAGGAGATCAAAACCCAGAAGGTCCGCATCGAAGGCTCCCTGTGGT
GGACCTACACAAGCAGCATCTTCTTCCGGGTCATCTTCGAAGCCGCCTTCATGTACGTCTTC
TATGTCATGTACGACGGCTTCTCCATGCAGCGGCTGGTGAAGTGCAACGCCTGGCCTTGTCC
CAACACTGTGGACTGCTTTGTGTCCCGGCCCACGGAGAAGACTGTCTTCACAGTGTTCATGA
TTGCAGTGTCTGGAATTTGCATCCTGCTGAATGTCACTGAATTGTGTTATTTGCTAATTAGA
TATTGTTCTGGGAAGTCAAAAAAGCCAGTTTAAAGGCGCGCCACCCCTGCAGGGAATTCCGC
ATTGCCCAGTTGTTAGATTAAGAAATAGACAGCATGAGAGGGATGAGGCAACCCGTGCTCAG
CTGTCAAGGCTCAGTCGCTAGCATTTCCCAACACAAAGATTCTGACCTTAAATGCAACCATT
TGAAACCCCTGTAGGCCTCAGGTGAAACTCCAGATGCCACAATGGAGCTCTGCTCCCCTAAA
GCCTCAAAACAAAGGCCTAATTCTATGCCTGTCTTAATTTTCTTTCACTTAAGTTAGTTCCA
CTGAGACCCCAGGCTGTTAGGGGTTATTGGTGTAAGGTACTTTCATATTTTAAACAGAGGAT
ATCGGCATTTGTTTCTTTCTCTGAGGACAAGAGAAAAAAGCCAGGTTCCACAGAGGACACAG
AGAAGGTTTGGGTGTCCTCCTGGGGTTCTTTTTGCCAACTTTCCCCACGTTAAAGGTGAACA
TTGGTTCTTTCATTTGCTTTGGAAGTTTTAATCTCTAACAGTGGACAAAGTTACCAGTGCCT
TAAACTCTGTTACACTTTTTGGAAGTGAAAACTTTGTAGTATGATAGGTTATTTTGATGTAA
AGATGTTCTGGATACCATTATATGTTCCCCCTGTTTCAGAGGCTCAGATTGTAATATGTAAA
TGGTATGTCATTCGCTACTATGATTTAATTTGAAATATGGTCTTTTGGTTATGAATACTTTG
CAGCACAGCTGAGAGGCTGTCTGTTGTATTCATTGTGGTCATAGCACCTAACAACATTGTAG
CCTCAATCGAGTGAGACAGACTAGAAGTTCCTAGTGATGGCTTATGATAGCAAATGGCCTCA
TGTCAAATATTTAGATGTAATTTTGTGTAAGAAATACAGACTGGATGTACCACCAACTACTA
CCTGTAATGACAGGCCTGTCCAACACATCTCCCTTTTCCATGACTGTGGTAGCCAGCATCGG
AAAGAACGCTGATTTAAAGAGGTCGCTTGGGAATTTTATTGACACAGTACCATTTAATGGGG
AGGACAAAATGGGGCAGGGGAGGGAGAAGTTTCTGTCGTTAAAAACAGATTTGGAAAGACTG
CTTCAGCCTCCAATTTTTTAAGTGAAAATATAGCTAATAACATGTGAAAAGAATAGAAGCTA
AGGTTTAGATAAATATTGAGCAGATCTATAGGAAGATTGAACCTGAATATTGCCATTATGCT
TCAAGAATAGCATTGTAAAAGCATTTTGTAATAATAAAGAATAGCTTTAATGATATGCTTGT
AACTAAAATAATTTTGTAATGTATCAAATACATTTAAAACATTAAAATATAATCTCTATAAT
AATTTAAAATCTAATATGGTTTTAATAGAACAGCGATATCAAGCTTATCGATAATCAACCTC
TGGATTACAAAATTTGTGAAAGATTGACTGGTATTCTTAACTATGTTGCTCCTTTTACGCTA
TGTGGATACGCTGCTTTAATGCCTTTGTATCATGCTATTGCTTCCCGTATGGCTTTCATTTT
AACGTGGCGTGGTGTGCACTGTGTTTGCTGACGCAACCCCCACTGGTTGGGGCATTGCCACC
ACCTGTCAGCTCCTTTCCGGGACTTTCGCTTTCCCCCTCCCTATTGCCACGGCGGAACTCAT
CGCCGCCTGCCTTGCCCGCTGCTGGACAGGGGCTCGGCTGTTGGGCACTGACAATTCCGTGG
TGTTGTCGGGGAAATCATCGTCCTTTCCTTGGCTGCTCGCCTATGTTGCCACCTGGATTCTG
CGCGGGACGTCCTTCTGCTACGTCCCTTCGGCCCTCAATCCAGCGGACCTTCCTTCCCGCGG
CCTGCTGCCGGCTCTGCGGCCTCTTCCGCGTCTTCGCCTTCGCCCTCAGACGAGTCGGATCT
CCCTTTGGGCCGCCTCCCCGCGAATTCATCGATACCGAGCGCTGCTCGAGAGATCTGTGATA
GCGGCCATCAAGCTGGCTGTGCCTTCTAGTTGCCAGCCATCTGTTGTTTGCCCCTCCCCCGT
GCCTTCCTTGACCCTGGAAGGTGCCACTCCCACTGTCCTTTCCTAATAAAATGAGGAAATTG
CATCGCATTGTCTGAGTAGGTGTCATTCTATTCTGGGGGGTGGGGTGGGGCAGGACAGCAAG
GGGGAGGATTGGGAAGACAATAGCAGGCATGCTGGGGACACGTGCGGACCGAGCGGCCGCAG
GAACCCCTAGTGATGGAGTTGGCCACTCCCTCTCTGCGCGCTCGCTCGCTCACTGAGGCCGG
GCGACCAAAGGTCGCCCGACGCCCGGGCTTTGCCCGGGCGGCCTCAGTGAGCGAGCGAGCGC
GCAGCTGCCTGCAGGGGCGCCTGATGCGGTATTTTCTCCTTACGCATCTGTGCGGTATTTCA
CACCGCATACGTCAAAGCAACCATAGTACGCGCCCTGTAGCGGCGCATTAAGCGCGGCGGGT
GTGGTGGTTACGCGCAGCGTGACCGCTACACTTGCCAGCGCCCTAGCGCCCGCTCCTTTCGC
TTTCTTCCCTTCCTTTCTCGCCACGTTCGCCGGCTTTCCCCGTCAAGCTCTAAATCGGGGGC
TCCCTTTAGGGTTCCGATTTAGTGCTTTACGGCACCTCGACCCCAAAAAACTTGATTTGGGT
GATGGTTCACGTAGTGGGCCATCGCCCTGATAGACGGTTTTTCGCCCTTTGACGTTGGAGTC
CACGTTCTTTAATAGTGGACTCTTGTTCCAAACTGGAACAACACTCAACCCTATCTCGGGCT
ATTCTTTTGATTTATAAGGGATTTTGCCGATTTCGGCCTATTGGTTAAAAAATGAGCTGATT
TAACAAAAATTTAACGCGAATTTTAACAAAATATTAACGTTTACAATTTTATGGTGCACTCT
CAGTACAATCTGCTCTGATGCCGCATAGTTAAGCCAGCCCCGACACCCGCCAACACCCGCTG
ACGCGCCCTGACGGGCTTGTCTGCTCCCGGCATCCGCTTACAGACAAGCTGTGACCGTCTCC
GGGAGCTGCATGTGTCAGAGGTTTTCACCGTCATCACCGAAACGCGCGAGACGAAAGGGCCT
CGTGATACGCCTATTTTTATAGGTTAATGTCATGATAATAATGGTTTCTTAGACGTCAGGTG
GCACTTTTCGGGGAAATGTGCGCGGAACCCCTATTTGTTTATTTTTCTAAATACATTCAAAT
ATGTATCCGCTCATGAGACAATAACCCTGATAAATGCTTCAATAATATTGAAAAAGGAAGAG
TATGAGTATTCAACATTTCCGTGTCGCCCTTATTCCCTTTTTTGCGGCATTTTGCCTTCCTG
TTTTTGCTCACCCAGAAACGCTGGTGAAAGTAAAAGATGCTGAAGATCAGTTGGGTGCACGA
GTGGGTTACATCGAACTGGATCTCAACAGCGGTAAGATCCTTGAGAGTTTTCGCCCCGAAGA
ACGTTTTCCAATGATGAGCACTTTTAAAGTTCTGCTATGTGGCGCGGTATTATCCCGTATTG
ACGCCGGGCAAGAGCAACTCGGTCGCCGCATACACTATTCTCAGAATGACTTGGTTGAGTAC
TCACCAGTCACAGAAAAGCATCTTACGGATGGCATGACAGTAAGAGAATTATGCAGTGCTGC
CATAACCATGAGTGATAACACTGCGGCCAACTTACTTCTGACAACGATCGGAGGACCGAAGG
AGCTAACCGCTTTTTTGCACAACATGGGGGATCATGTAACTCGCCTTGATCGTTGGGAACCG
GAGCTGAATGAAGCCATACCAAACGACGAGCGTGACACCA

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 66. An exemplary nucleotide sequence for vector c81.1 encoding mouse GJB2 is set forth in SEQ ID NO: 66 (hGJB2 GRE1 underlined; mouse GJB2 coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTA
AGAGAGCACTTGGGAAGAGCCCCCGAGGGCAGCCGGGGCTTGCCGCCTCACCCTTTTGGTTT
CACATCCCAGAAATCAGTAAGGCAGGAATTGGAGGCTGCTTCTTGCCTTAGCAACTCGGTGA
CCTTAGGCAGAACAGTTCAGCCTTCTGAGTGTCCTTCCTCTTCTGTAAGGGGAGCGTAAACC
GTCCTCCATGCAGAACGTGTACTGTGCCTGGCACAGCACTGGGGCATTAGGATCTCCAAATT
AAAGGCTCACTCTGCGGGATGGAGGCAGCCACAGCTGGAAGAAGGAACATTTGGGGCCAGAA
GTCCCCCTACCTCCGTCCTAAGAGAGAAGATGGGAATAACGACCCTCGCTGAAATGATTGCT
CTCTGGCCAGCTCGCCTCGCATCCACATCCAAATCTGGGAGGCACAGAGCGCATCAGGACAT
CGGGTTCTGTCAGTGTAATGGGCGTGGCTCCTGACCTTCTGTCTGTATCAGAGAAGATAAGG
GAGAACATTTGAAAGAAAGGAGAAAGAAGATAGCCACTGGAGAACAGAGCAAAGGAGCCAGC
AGAAAAAGACGAGACGGCTGTAGCCCCACAGGAAGCAGAAACCGATAGGCTAAGTAGGATAC
ACACAAAGAAAAGTAGATCCCGAGAGGCATTTCCCCGAGGGCTTTCATGTGGTTTCTCGTGA
GGAGAAGCTGACTGCAGGGTGTTTGAAAGAACGACTTATGCAGCCATAAAAAATGATGAGTT
CATGTCCTTTGTAGGGACATGGATGATTAATTAAGACCTCGAAGGGGACTTGGGGGGTTCGG
GGCTTTCGGGGGCGGTCGGGGGTTCGCGGACCCGGGAAGCTCTGAGGACCCAGAGGCCGGGC
GCGCTCCGCCCGCGGCGCCGCCCCCTCCGTAACTTTCCCAGTCTCCGAGGGAAGAGGCGGGG
TGTGGGGTGCGGTTAAAAGGCGCCACGGCGGGAGACAGGTGTTGCGGCCCCGCAGCGCCCGC
GCGCTCCTCTCCCCGACTCGGAGCCCCTCGGCGGCGCCCGGCCCAGGACCCGCCTAGGAGCG
CAGGAGCCCCAGCGCAGAGACCCCAACGCCGAGACCCCCGCCCCGGCCCCGCCGCGCTTCCT
CCCGACGCAGAGCAAACCGCCCAGAGTAGAAGCCATGGATTGGGGCACACTCCAGAGCATCC
TCGGGGGTGTCAACAAACACTCCACCAGCATTGGAAAGATCTGGCTCACGGTCCTCTTCATC
TTCCGCATCATGATCCTCGTGGTGGCTGCAAAGGAGGTGTGGGGAGATGAGCAAGCCGATTT
TGTCTGCAACACGCTCCAGCCTGGCTGCAAGAATGTATGCTACGACCACCACTTCCCCATCT
CTCACATCCGGCTCTGGGCTCTGCAGCTGATCATGGTGTCCACGCCAGCCCTCCTGGTAGCT
ATGCATGTGGCCTACCGGAGACATGAAAAGAAACGGAAGTTCATGAAGGGAGAGATAAAGAA
CGAGTTTAAGGACATCGAAGAGATCAAAACCCAGAAGGTCCGTATCGAAGGGTCCCTGTGGT
GGACCTACACCACCAGCATCTTCTTCCGGGTCATCTTTGAAGCCGTCTTCATGTACGTCTTT
TACATCATGTACAATGGCTTCTTCATGCAACGTCTGGTGAAATGCAACGCTTGGCCCTGCCC
CAATACAGTGGACTGCTTCATTTCCAGGCCCACAGAAAAGACTGTCTTCACCGTGTTTATGA
TTTCTGTGTCTGGAATTTGCATTCTGCTAAATATCACAGAGCTGTGCTATTTGTTCGTTAGG
TATTGCTCAGGAAAGTCCAAAAGACCAGTCTACCCATACGATGTTCCAGATTACGCTTAAAG
GCGCGCCACCCCTGCAGGGAATTCCGCATTGCCCAGTTGTTAGATTAAGAAATAGACAGCAT
GAGAGGGATGAGGCAACCCGTGCTCAGCTGTCAAGGCTCAGTCGCTAGCATTTCCCAACACA
GCCACAATGGAGCTCTGCTCCCCTAAAGCCTCAAAACAAAGGCCTAATTCTATGCCTGTCTT
AATTTTCTTTCACTTAAGTTAGTTCCACTGAGACCCCAGGCTGTTAGGGGTTATTGGTGTAA
GGTACTTTCATATTTTAAACAGAGGATATCGGCATTTGTTTCTTTCTCTGAGGACAAGAGAA
AAAAGCCAGGTTCCACAGAGGACACAGAGAAGGTTTGGGTGTCCTCCTGGGGTTCTTTTTGC
CAACTTTCCCCACGTTAAAGGTGAACATTGGTTCTTTCATTTGCTTTGGAAGTTTTAATCTC
TAACAGTGGACAAAGTTACCAGTGCCTTAAACTCTGTTACACTTTTTGGAAGTGAAAACTTT
GTAGTATGATAGGTTATTTTGATGTAAAGATGTTCTGGATACCATTATATGTTCCCCCTGTT
TCAGAGGCTCAGATTGTAATATGTAAATGGTATGTCATTCGCTACTATGATTTAATTTGAAA
TATGGTCTTTTGGTTATGAATACTTTGCAGCACAGCTGAGAGGCTGTCTGTTGTATTCATTG
TGGTCATAGCACCTAACAACATTGTAGCCTCAATCGAGTGAGACAGACTAGAAGTTCCTAGT
GATGGCTTATGATAGCAAATGGCCTCATGTCAAATATTTAGATGTAATTTTGTGTAAGAAAT
TTCCATGACTGTGGTAGCCAGCATCGGAAAGAACGCTGATTTAAAGAGGTCGCTTGGGAATT
TTATTGACACAGTACCATTTAATGGGGAGGACAAAATGGGGCAGGGGAGGGAGAAGTTTCTG
TCGTTAAAAACAGATTTGGAAAGACTGGACTCTAAAGTCTGTTGATTAAAGATGAGCTTTGT
AATAACATGTGAAAAGAATAGAAGCTAAGGTTTAGATAAATATTGAGCAGATCTATAGGAAG
ATTGAACCTGAATATTGCCATTATGCTTGACATGGTTTCCAAAAAATGGTACTCCACATATT
TCAGTGAGGGTAAGTATTTTCCTGTTGTCAAGAATAGCATTGTAAAAGCATTTTGTAATAAT
AAAGAATAGCTTTAATGATATGCTTGTAACTAAAATAATTTTGTAATGTATCAAATACATTT
AAAACATTAAAATATAATCTCTATAATAATTTAAAATCTAATATGGTTTTAATAGAACAGCG
ATATCAAGCTTATCGATAATCAACCTCTGGATTACAAAATTTGTGAAAGATTGACTGGTATT
CTTAACTATGTTGCTCCTTTTACGCTATGTGGATACGCTGCTTTAATGCCTTTGTATCATGC
TATTGCTTCCCGTATGGCTTTCATTTTCTCCTCCTTGTATAAATCCTGGTTGCTGTCTCTTT
ACCCCCACTGGTTGGGGCATTGCCACCACCTGTCAGCTCCTTTCCGGGACTTTCGCTTTCCC
CCTCCCTATTGCCACGGCGGAACTCATCGCCGCCTGCCTTGCCCGCTGCTGGACAGGGGCTC
GGCTGTTGGGCACTGACAATTCCGTGGTGTTGTCGGGGAAATCATCGTCCTTTCCTTGGCTG
CTCGCCTATGTTGCCACCTGGATTCTGCGCGGGACGTCCTTCTGCTACGTCCCTTCGGCCCT
CAATCCAGCGGACCTTCCTTCCCGCGGCCTGCTGCCGGCTCTGCGGCCTCTTCCGCGTCTTC
GCCTTCGCCCTCAGACGAGTCGGATCTCCCTTTGGGCCGCCTCCCCGCGAATTCATCGATAC
CGAGCGCTGCTCGAGAGATCTGTGATAGCGGCCATCAAGCTGGCTGTGCCTTCTAGTTGCCA
GCCATCTGTTGTTTGCCCCTCCCCCGTGCCTTCCTTGACCCTGGAAGGTGCCACTCCCACTG
TCCTTTCCTAATAAAATGAGGAAATTGCATCGCATTGTCTGAGTAGGTGTCATTCTATTCTG
GGGGGTGGGGTGGGGCAGGACAGCAAGGGGGAGGATTGGGAAGACAATAGCAGGCATGCTGG
GGACACGTGCGGACCGAGCGGCCGCAGGAACCCCTAGTGATGGAGTTGGCCACTCCCTCTCT
GCGCGCTCGCTCGCTCACTGAGGCCGGGCGACCAAAGGTCGCCCGACGCCCGGGCTTTGCCC
GGGCGGCCTCAGTGAGCGAGCGAGCGCGCAGCTGCCTGCAGGGGCGCCTGATGCGGTATTTT
CTCCTTACGCATCTGTGCGGTATTTCACACCGCATACGTCAAAGCAACCATAGTACGCGCCC
TGTAGCGGCGCATTAAGCGCGGCGGGTGTGGTGGTTACGCGCAGCGTGACCGCTACACTTGC
CAGCGCCCTAGCGCCCGCTCCTTTCGCTTTCTTCCCTTCCTTTCTCGCCACGTTCGCCGGCT
TTCCCCGTCAAGCTCTAAATCGGGGGCTCCCTTTAGGGTTCCGATTTAGTGCTTTACGGCAC
CTCGACCCCAAAAAACTTGATTTGGGTGATGGTTCACGTAGTGGGCCATCGCCCTGATAGAC
GGTTTTTCGCCCTTTGACGTTGGAGTCCACGTTCTTTAATAGTGGACTCTTGTTCCAAACTG
GAACAACACTCAACCCTATCTCGGGCTATTCTTTTGATTTATAAGGGATTTTGCCGATTTCG
GCCTATTGGTTAAAAAATGAGCTGATTTAACAAAAATTTAACGCGAATTTTAACAAAATATT
AACGTTTACAATTTTATGGTGCACTCTCAGTACAATCTGCTCTGATGCCGCATAGTTAAGCC
AGCCCCGACACCCGCCAACACCCGCTGACGCGCCCTGACGGGCTTGTCTGCTCCCGGCATCC
GCTTACAGACAAGCTGTGACCGTCTCCGGGAGCTGCATGTGTCAGAGGTTTTCACCGTCATC
ACCGAAACGCGCGAGACGAAAGGGCCTCGTGATACGCCTATTTTTATAGGTTAATGTCATGA
TAATAATGGTTTCTTAGACGTCAGGTGGCACTTTTCGGGGAAATGTGCGCGGAACCCCTATT
TGTTTATTTTTCTAAATACATTCAAATATGTATCCGCTCATGAGACAATAACCCTGATAAAT
GCTTCAATAATATTGAAAAAGGAAGAGTATGAGTATTCAACATTTCCGTGTCGCCCTTATTC
CCTTTTTTGCGGCATTTTGCCTTCCTGTTTTTGCTCACCCAGAAACGCTGGTGAAAGTAAAA
GATGCTGAAGATCAGTTGGGTGCACGAGTGGGTTACATCGAACTGGATCTCAACAGCGGTAA
GATCCTTGAGAGTTTTCGCCCCGAAGAACGTTTTCCAATGATGAGCACTTTTAAAGTTCTGC
TATGTGGCGCGGTATTATCCCGTATTGACGCCGGGCAAGAGCAACTCGGTCGCCGCATACAC
TATTCTCAGAATGACTTGGTTGAGTACTCACCAGTCACAGAAAAGCATCTTACGGATGGCAT
GACAGTAAGAGAATTATGCAGTGCTGCCATAACCATGAGTGATAACACTGCGGCCAACTTAC
TTCTGACAACGATCGGAGGACCGAAGGAGCTAACCGCTTTTTTGCACAACATGGGGGATCAT
GTAACTCGCCTTGATCGTTGGGAACCGGAGCTGAATGAAGCCATACCAAACGACGAGCGTGA
CACCA

In some embodiments, an AAV vector described herein comprises an AAV 5′ ITR, a GJB2 GRE enhancer (hGJB2 GRE2), a GJB2 basal promoter, a GJB2 exon 1 5′ UTR, Kozak sequence, nucleotide sequence encoding a gene product (e.g., GJB2 or GFP), a GJB2 exon 2 3′ UTR, a WPRE, a bovine growth hormone poly A signal, and an AAV 3′ ITR (e.g., vector c81.2).

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 48. An exemplary nucleotide sequence for vector c81.2 encoding eGFP is set forth in SEQ ID NO: 48 (hGJB2 GRE2 underlined; eGFP coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTC
AGTGATGCCTGAAACCTCAGATGGTACTGAACCCTCTATATAATCTGTTTTTTCCTATACAT
ACAAACCTACCATAAGGCTTAATGGTAAGAGATTAACAATAAAGAATAATAAAACAACACTT
ATAACAATGTATAACAATATATTGTAATATAAGTTTTTGGATGCAGTCTCTCTCTCAAAATG
CTATCATATTTTCCAACTGTGGTTGACTACAGGTAACTGGAACCACAAAAATGAAACAGTGG
ATAAGAGGGCGACTCCTGTACCAAAGAAAAAAATAGAGTGTTGCAGCTGTAACATAGTTGAA
TGACTGAGTTAGACTGCATAACTGACACACAAAACCACATAAATATAAATGAAGGAATCTCT
GGGTGTAATCTGGTGCAAAGGTGACTGTGTTAATCATTAATCCACAAGTTGCTATCCTGAAG
TGTGCCAAATGCTTTATGTTTATTTCATCACATAGCTCTATAAAGAAAGGATTTGTAATTCC
TTTCTACAGAAGTGGAAAGTAAGTCTTAAGACTCAAAAAACTTTAAAAACTACAATGAAGTA
ACAACTTTTATTAATTTATTTTGTGTCTTTCCAGAATTTCTATATATATAGGAATGTGATAT
GAATCTATATGTGAATTGAATCTACATGAATATTGATGACTTTTATTTCCCCTTTTGCACAT
AAGATAGAATATTTTACCTACTATTCCACACTTTGCTTTTCTTAACATATCATGGGATCTTT
TTATATAAGTGAACAAAGAGTTTCTTCATTCTTTCACACAGTTTAATTAAGACCTCGAAGGG
GACTTGGGGGGTTCGGGGCTTTCGGGGGCGGTCGGGGGTTCGCGGACCCGGGAAGCTCTGAG
GACCCAGAGGCCGGGCGCGCTCCGCCCGCGGCGCCGCCCCCTCCGTAACTTTCCCAGTCTCC
GAGGGAAGAGGCGGGGTGTGGGGTGCGGTTAAAAGGCGCCACGGCGGGAGACAGGTGTTGCG
GCCCCGCAGCGCCCGCGCGCTCCTCTCCCCGACTCGGAGCCCCTCGGCGGCGCCCGGCCCAG
GACCCGCCTAGGAGCGCAGGAGCCCCAGCGCAGAGACCCCAACGCCGAGACCCCCGCCCCGG
CCCCGCCGCGCTTCCTCCCGACGCAGAGCAAACCGCCCAGAGTAGAAGCCATGGTGAGCAAG
GGCGAGGAGCTGTTCACCGGGGTGGTGCCCATCCTGGTCGAGCTGGACGGCGACGTAAACGG
CCACAAGTTCAGCGTGTCCGGCGAGGGCGAGGGCGATGCCACCTACGGCAAGCTGACCCTGA
AGTTCATCTGCACCACCGGCAAGCTGCCCGTGCCCTGGCCCACCCTCGTGACCACCCTGACC
TACGGCGTGCAGTGCTTCAGCCGCTACCCCGACCACATGAAGCAGCACGACTTCTTCAAGTC
CGCCATGCCCGAAGGCTACGTCCAGGAGCGCACCATCTTCTTCAAGGACGACGGCAACTACA
AGACCCGCGCCGAGGTGAAGTTCGAGGGCGACACCCTGGTGAACCGCATCGAGCTGAAGGGC
ATCGACTTCAAGGAGGACGGCAACATCCTGGGGCACAAGCTGGAGTACAACTACAACAGCCA
CAACGTCTATATCATGGCCGACAAGCAGAAGAACGGCATCAAGGTGAACTTCAAGATCCGCC
ACAACATCGAGGACGGCAGCGTGCAGCTCGCCGACCACTACCAGCAGAACACCCCCATCGGC
GACGGCCCCGTGCTGCTGCCCGACAACCACTACCTGAGCACCCAGTCCGCCCTGAGCAAAGA
CCCCAACGAGAAGCGCGATCACATGGTCCTGCTGGAGTTCGTGACCGCCGCCGGGATCACTC
TCGGCATGGACGAGCTGTACAAGTAAAGGCGCGCCACCCCTGCAGGGAATTCCGCATTGCCC
AGTTGTTAGATTAAGAAATAGACAGCATGAGAGGGATGAGGCAACCCGTGCTCAGCTGTCAA
GGCTCAGTCGCTAGCATTTCCCAACACAAAGATTCTGACCTTAAATGCAACCATTTGAAACC
CCTGTAGGCCTCAGGTGAAACTCCAGATGCCACAATGGAGCTCTGCTCCCCTAAAGCCTCAA
AACAAAGGCCTAATTCTATGCCTGTCTTAATTTTCTTTCACTTAAGTTAGTTCCACTGAGAC
CCCAGGCTGTTAGGGGTTATTGGTGTAAGGTACTTTCATATTTTAAACAGAGGATATCGGCA
TTTGTTTCTTTCTCTGAGGACAAGAGAAAAAAGCCAGGTTCCACAGAGGACACAGAGAAGGT
TTGGGTGTCCTCCTGGGGTTCTTTTTGCCAACTTTCCCCACGTTAAAGGTGAACATTGGTTC
TTTCATTTGCTTTGGAAGTTTTAATCTCTAACAGTGGACAAAGTTACCAGTGCCTTAAACTC
TGTTACACTTTTTGGAAGTGAAAACTTTGTAGTATGATAGGTTATTTTGATGTAAAGATGTT
CTGGATACCATTATATGTTCCCCCTGTTTCAGAGGCTCAGATTGTAATATGTAAATGGTATG
TCATTCGCTACTATGATTTAATTTGAAATATGGTCTTTTGGTTATGAATACTTTGCAGCACA
GCTGAGAGGCTGTCTGTTGTATTCATTGTGGTCATAGCACCTAACAACATTGTAGCCTCAAT
CGAGTGAGACAGACTAGAAGTTCCTAGTGATGGCTTATGATAGCAAATGGCCTCATGTCAAA
TATTTAGATGTAATTTTGTGTAAGAAATACAGACTGGATGTACCACCAACTACTACCTGTAA
TGACAGGCCTGTCCAACACATCTCCCTTTTCCATGACTGTGGTAGCCAGCATCGGAAAGAAC
GCTGATTTAAAGAGGTCGCTTGGGAATTTTATTGACACAGTACCATTTAATGGGGAGGACAA
AATGGGGCAGGGGAGGGAGAAGTTTCTGTCGTTAAAAACAGATTTGGAAAGACTGGACTCTA
AAGTCTGTTGATTAAAGATGAGCTTTGTCTACTTCAAAAGTTTGTTTGCTTACCCCTTCAGC
CTCCAATTTTTTAAGTGAAAATATAGCTAATAACATGTGAAAAGAATAGAAGCTAAGGTTTA
GATAAATATTGAGCAGATCTATAGGAAGATTGAACCTGAATATTGCCATTATGCTTGACATG
GTTTCCAAAAAATGGTACTCCACATATTTCAGTGAGGGTAAGTATTTTCCTGTTGTCAAGAA
TAGCATTGTAAAAGCATTTTGTAATAATAAAGAATAGCTTTAATGATATGCTTGTAACTAAA
ATAATTTTGTAATGTATCAAATACATTTAAAACATTAAAATATAATCTCTATAATAATTTAA
AATCTAATATGGTTTTAATAGAACAGCGATATCAAGCTTATCGATAATCAACCTCTGGATTA
CAAAATTTGTGAAAGATTGACTGGTATTCTTAACTATGTTGCTCCTTTTACGCTATGTGGAT
ACGCTGCTTTAATGCCTTTGTATCATGCTATTGCTTCCCGTATGGCTTTCATTTTCTCCTCC
CGTGGTGTGCACTGTGTTTGCTGACGCAACCCCCACTGGTTGGGGCATTGCCACCACCTGTC
AGCTCCTTTCCGGGACTTTCGCTTTCCCCCTCCCTATTGCCACGGCGGAACTCATCGCCGCC
TGCCTTGCCCGCTGCTGGACAGGGGCTCGGCTGTTGGGCACTGACAATTCCGTGGTGTTGTC
GGGGAAATCATCGTCCTTTCCTTGGCTGCTCGCCTATGTTGCCACCTGGATTCTGCGCGGGA
CGTCCTTCTGCTACGTCCCTTCGGCCCTCAATCCAGCGGACCTTCCTTCCCGCGGCCTGCTG
CCGGCTCTGCGGCCTCTTCCGCGTCTTCGCCTTCGCCCTCAGACGAGTCGGATCTCCCTTTG
GGCCGCCTCCCCGCGAATTCATCGATACCGAGCGCTGCTCGAGAGATCTGTGATAGCGGCCA
TCAAGCTGGCTGTGCCTTCTAGTTGCCAGCCATCTGTTGTTTGCCCCTCCCCCGTGCCTTCC
TTGACCCTGGAAGGTGCCACTCCCACTGTCCTTTCCTAATAAAATGAGGAAATTGCATCGCA
TTGTCTGAGTAGGTGTCATTCTATTCTGGGGGGTGGGGTGGGGCAGGACAGCAAGGGGGAGG
ATTGGGAAGACAATAGCAGGCATGCTGGGGACACGTGCGGACCGAGCGGCCGCAGGAACCCC
TAGTGATGGAGTTGGCCACTCCCTCTCTGCGCGCTCGCTCGCTCACTGAGGCCGGGCGACCA
AAGGTCGCCCGACGCCCGGGCTTTGCCCGGGCGGCCTCAGTGAGCGAGCGAGCGCGCAGCTG
CCTGCAGGGGCGCCTGATGCGGTATTTTCTCCTTACGCATCTGTGCGGTATTTCACACCGCA
TACGTCAAAGCAACCATAGTACGCGCCCTGTAGCGGCGCATTAAGCGCGGCGGGTGTGGTGG
TTACGCGCAGCGTGACCGCTACACTTGCCAGCGCCCTAGCGCCCGCTCCTTTCGCTTTCTTC
CCTTCCTTTCTCGCCACGTTCGCCGGCTTTCCCCGTCAAGCTCTAAATCGGGGGCTCCCTTT
AGGGTTCCGATTTAGTGCTTTACGGCACCTCGACCCCAAAAAACTTGATTTGGGTGATGGTT
CACGTAGTGGGCCATCGCCCTGATAGACGGTTTTTCGCCCTTTGACGTTGGAGTCCACGTTC
TTTAATAGTGGACTCTTGTTCCAAACTGGAACAACACTCAACCCTATCTCGGGCTATTCTTT
TGATTTATAAGGGATTTTGCCGATTTCGGCCTATTGGTTAAAAAATGAGCTGATTTAACAAA
AATTTAACGCGAATTTTAACAAAATATTAACGTTTACAATTTTATGGTGCACTCTCAGTACA
ATCTGCTCTGATGCCGCATAGTTAAGCCAGCCCCGACACCCGCCAACACCCGCTGACGCGCC
CTGACGGGCTTGTCTGCTCCCGGCATCCGCTTACAGACAAGCTGTGACCGTCTCCGGGAGCT
GCATGTGTCAGAGGTTTTCACCGTCATCACCGAAACGCGCGAGACGAAAGGGCCTCGTGATA
CGCCTATTTTTATAGGTTAATGTCATGATAATAATGGTTTCTTAGACGTCAGGTGGCACTTT
TCGGGGAAATGTGCGCGGAACCCCTATTTGTTTATTTTTCTAAATACATTCAAATATGTATC
CGCTCATGAGACAATAACCCTGATAAATGCTTCAATAATATTGAAAAAGGAAGAGTATGAGT
ATTCAACATTTCCGTGTCGCCCTTATTCCCTTTTTTGCGGCATTTTGCCTTCCTGTTTTTGC
TCACCCAGAAACGCTGGTGAAAGTAAAAGATGCTGAAGATCAGTTGGGTGCACGAGTGGGTT
ACATCGAACTGGATCTCAACAGCGGTAAGATCCTTGAGAGTTTTCGCCCCGAAGAACGTTTT
CCAATGATGAGCACTTTTAAAGTTCTGCTATGTGGCGCGGTATTATCCCGTATTGACGCCGG
GCAAGAGCAACTCGGTCGCCGCATACACTATTCTCAGAATGACTTGGTTGAGTACTCACCAG
TCACAGAAAAGCATCTTACGGATGGCATGACAGTAAGAGAATTATGCAGTGCTGCCATAACC
ATGAGTGATAACACTGCGGCCAACTTACTTCTGACAACGATCGGAGGACCGAAGGAGCTAAC
CGCTTTTTTGCACAACATGGGGGATCATGTAACTCGCCTTGATCGTTGGGAACCGGAGCTGA
ATGAAGCCATACCAAACGACGAGCGTGACACCA

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 67. An exemplary nucleotide sequence for vector c81.2 encoding human GJB2 is set forth in SEQ ID NO: 67 (hGJB2 GRE2 underlined; human GJB2 coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTC
AGTGATGCCTGAAACCTCAGATGGTACTGAACCCTCTATATAATCTGTTTTTTCCTATACAT
ACAAACCTACCATAAGGCTTAATGGTAAGAGATTAACAATAAAGAATAATAAAACAACACTT
ATAACAATGTATAACAATATATTGTAATATAAGTTTTTGGATGCAGTCTCTCTCTCAAAATG
CTATCATATTTTCCAACTGTGGTTGACTACAGGTAACTGGAACCACAAAAATGAAACAGTGG
ATAAGAGGGCGACTCCTGTACCAAAGAAAAAAATAGAGTGTTGCAGCTGTAACATAGTTGAA
TGACTGAGTTAGACTGCATAACTGACACACAAAACCACATAAATATAAATGAAGGAATCTCT
GGGTGTAATCTGGTGCAAAGGTGACTGTGTTAATCATTAATCCACAAGTTGCTATCCTGAAG
TGTGCCAAATGCTTTATGTTTATTTCATCACATAGCTCTATAAAGAAAGGATTTGTAATTCC
TTTCTACAGAAGTGGAAAGTAAGTCTTAAGACTCAAAAAACTTTAAAAACTACAATGAAGTA
ACAACTTTTATTAATTTATTTTGTGTCTTTCCAGAATTTCTATATATATAGGAATGTGATAT
GAATCTATATGTGAATTGAATCTACATGAATATTGATGACTTTTATTTCCCCTTTTGCACAT
AAGATAGAATATTTTACCTACTATTCCACACTTTGCTTTTCTTAACATATCATGGGATCTTT
TTATATAAGTGAACAAAGAGTTTCTTCATTCTTTCACACAGTTTAATTAAGACCTCGAAGGG
GACTTGGGGGGTTCGGGGCTTTCGGGGGCGGTCGGGGGTTCGCGGACCCGGGAAGCTCTGAG
GACCCAGAGGCCGGGCGCGCTCCGCCCGCGGCGCCGCCCCCTCCGTAACTTTCCCAGTCTCC
GAGGGAAGAGGCGGGGTGTGGGGTGCGGTTAAAAGGCGCCACGGCGGGAGACAGGTGTTGCG
GCCCCGCAGCGCCCGCGCGCTCCTCTCCCCGACTCGGAGCCCCTCGGCGGCGCCCGGCCCAG
GACCCGCCTAGGAGCGCAGGAGCCCCAGCGCAGAGACCCCAACGCCGAGACCCCCGCCCCGG
CCCCGCCGCGCTTCCTCCCGACGCAGAGCAAACCGCCCAGAGTAGAAGCCATGGATTGGGGC
ACGCTGCAGACGATCCTGGGGGGTGTGAACAAACACTCCACCAGCATTGGAAAGATCTGGCT
CACCGTCCTCTTCATTTTTCGCATTATGATCCTCGTTGTGGCTGCAAAGGAGGTGTGGGGAG
ATGAGCAGGCCGACTTTGTCTGCAACACCCTGCAGCCAGGCTGCAAGAACGTGTGCTACGAT
CACTACTTCCCCATCTCCCACATCCGGCTATGGGCCCTGCAGCTGATCTTCGTGTCCACGCC
AGCGCTCCTAGTGGCCATGCACGTGGCCTACCGGAGACATGAGAAGAAGAGGAAGTTCATCA
AGGGGGAGATAAAGAGTGAATTTAAGGACATCGAGGAGATCAAAACCCAGAAGGTCCGCATC
GAAGGCTCCCTGTGGTGGACCTACACAAGCAGCATCTTCTTCCGGGTCATCTTCGAAGCCGC
CTTCATGTACGTCTTCTATGTCATGTACGACGGCTTCTCCATGCAGCGGCTGGTGAAGTGCA
ACGCCTGGCCTTGTCCCAACACTGTGGACTGCTTTGTGTCCCGGCCCACGGAGAAGACTGTC
TTCACAGTGTTCATGATTGCAGTGTCTGGAATTTGCATCCTGCTGAATGTCACTGAATTGTG
TTATTTGCTAATTAGATATTGTTCTGGGAAGTCAAAAAAGCCAGTTTAAAGGCGCGCCACCC
CTGCAGGGAATTCCGCATTGCCCAGTTGTTAGATTAAGAAATAGACAGCATGAGAGGGATGA
GGCAACCCGTGCTCAGCTGTCAAGGCTCAGTCGCTAGCATTTCCCAACACAAAGATTCTGAC
CTTAAATGCAACCATTTGAAACCCCTGTAGGCCTCAGGTGAAACTCCAGATGCCACAATGGA
GCTCTGCTCCCCTAAAGCCTCAAAACAAAGGCCTAATTCTATGCCTGTCTTAATTTTCTTTC
ACTTAAGTTAGTTCCACTGAGACCCCAGGCTGTTAGGGGTTATTGGTGTAAGGTACTTTCAT
ATTTTAAACAGAGGATATCGGCATTTGTTTCTTTCTCTGAGGACAAGAGAAAAAAGCCAGGT
TCCACAGAGGACACAGAGAAGGTTTGGGTGTCCTCCTGGGGTTCTTTTTGCCAACTTTCCCC
ACGTTAAAGGTGAACATTGGTTCTTTCATTTGCTTTGGAAGTTTTAATCTCTAACAGTGGAC
AAAGTTACCAGTGCCTTAAACTCTGTTACACTTTTTGGAAGTGAAAACTTTGTAGTATGATA
GGTTATTTTGATGTAAAGATGTTCTGGATACCATTATATGTTCCCCCTGTTTCAGAGGCTCA
GATTGTAATATGTAAATGGTATGTCATTCGCTACTATGATTTAATTTGAAATATGGTCTTTT
GGTTATGAATACTTTGCAGCACAGCTGAGAGGCTGTCTGTTGTATTCATTGTGGTCATAGCA
CCTAACAACATTGTAGCCTCAATCGAGTGAGACAGACTAGAAGTTCCTAGTGATGGCTTATG
ATAGCAAATGGCCTCATGTCAAATATTTAGATGTAATTTTGTGTAAGAAATACAGACTGGAT
GTACCACCAACTACTACCTGTAATGACAGGCCTGTCCAACACATCTCCCTTTTCCATGACTG
TGGTAGCCAGCATCGGAAAGAACGCTGATTTAAAGAGGTCGCTTGGGAATTTTATTGACACA
GTACCATTTAATGGGGAGGACAAAATGGGGCAGGGGAGGGAGAAGTTTCTGTCGTTAAAAAC
AGATTTGGAAAGACTGGACTCTAAAGTCTGTTGATTAAAGATGAGCTTTGTCTACTTCAAAA
GTTTGTTTGCTTACCCCTTCAGCCTCCAATTTTTTAAGTGAAAATATAGCTAATAACATGTG
AAAAGAATAGAAGCTAAGGTTTAGATAAATATTGAGCAGATCTATAGGAAGATTGAACCTGA
ATATTGCCATTATGCTTGACATGGTTTCCAAAAAATGGTACTCCACATATTTCAGTGAGGGT
AAGTATTTTCCTGTTGTCAAGAATAGCATTGTAAAAGCATTTTGTAATAATAAAGAATAGCT
TTAATGATATGCTTGTAACTAAAATAATTTTGTAATGTATCAAATACATTTAAAACATTAAA
ATATAATCTCTATAATAATTTAAAATCTAATATGGTTTTAATAGAACAGCGATATCAAGCTT
ATCGATAATCAACCTCTGGATTACAAAATTTGTGAAAGATTGACTGGTATTCTTAACTATGT
TGCTCCTTTTACGCTATGTGGATACGCTGCTTTAATGCCTTTGTATCATGCTATTGCTTCCC
GTATGGCTTTCATTTTCTCCTCCTTGTATAAATCCTGGTTGCTGTCTCTTTATGAGGAGTTG
TGGCCCGTTGTCAGGCAACGTGGCGTGGTGTGCACTGTGTTTGCTGACGCAACCCCCACTGG
TTGGGGCATTGCCACCACCTGTCAGCTCCTTTCCGGGACTTTCGCTTTCCCCCTCCCTATTG
CCACGGCGGAACTCATCGCCGCCTGCCTTGCCCGCTGCTGGACAGGGGCTCGGCTGTTGGGC
ACTGACAATTCCGTGGTGTTGTCGGGGAAATCATCGTCCTTTCCTTGGCTGCTCGCCTATGT
TGCCACCTGGATTCTGCGCGGGACGTCCTTCTGCTACGTCCCTTCGGCCCTCAATCCAGCGG
ACCTTCCTTCCCGCGGCCTGCTGCCGGCTCTGCGGCCTCTTCCGCGTCTTCGCCTTCGCCCT
CAGACGAGTCGGATCTCCCTTTGGGCCGCCTCCCCGCGAATTCATCGATACCGAGCGCTGCT
CGAGAGATCTGTGATAGCGGCCATCAAGCTGGCTGTGCCTTCTAGTTGCCAGCCATCTGTTG
TTTGCCCCTCCCCCGTGCCTTCCTTGACCCTGGAAGGTGCCACTCCCACTGTCCTTTCCTAA
TAAAATGAGGAAATTGCATCGCATTGTCTGAGTAGGTGTCATTCTATTCTGGGGGGTGGGGT
GGGGCAGGACAGCAAGGGGGAGGATTGGGAAGACAATAGCAGGCATGCTGGGGACACGTGCG
GACCGAGCGGCCGCAGGAACCCCTAGTGATGGAGTTGGCCACTCCCTCTCTGCGCGCTCGCT
CGCTCACTGAGGCCGGGCGACCAAAGGTCGCCCGACGCCCGGGCTTTGCCCGGGCGGCCTCA
GTGAGCGAGCGAGCGCGCAGCTGCCTGCAGGGGCGCCTGATGCGGTATTTTCTCCTTACGCA
TCTGTGCGGTATTTCACACCGCATACGTCAAAGCAACCATAGTACGCGCCCTGTAGCGGCGC
ATTAAGCGCGGCGGGTGTGGTGGTTACGCGCAGCGTGACCGCTACACTTGCCAGCGCCCTAG
CGCCCGCTCCTTTCGCTTTCTTCCCTTCCTTTCTCGCCACGTTCGCCGGCTTTCCCCGTCAA
GCTCTAAATCGGGGGCTCCCTTTAGGGTTCCGATTTAGTGCTTTACGGCACCTCGACCCCAA
AAAACTTGATTTGGGTGATGGTTCACGTAGTGGGCCATCGCCCTGATAGACGGTTTTTCGCC
CTTTGACGTTGGAGTCCACGTTCTTTAATAGTGGACTCTTGTTCCAAACTGGAACAACACTC
AACCCTATCTCGGGCTATTCTTTTGATTTATAAGGGATTTTGCCGATTTCGGCCTATTGGTT
AAAAAATGAGCTGATTTAACAAAAATTTAACGCGAATTTTAACAAAATATTAACGTTTACAA
TTTTATGGTGCACTCTCAGTACAATCTGCTCTGATGCCGCATAGTTAAGCCAGCCCCGACAC
CCGCCAACACCCGCTGACGCGCCCTGACGGGCTTGTCTGCTCCCGGCATCCGCTTACAGACA
AGCTGTGACCGTCTCCGGGAGCTGCATGTGTCAGAGGTTTTCACCGTCATCACCGAAACGCG
CGAGACGAAAGGGCCTCGTGATACGCCTATTTTTATAGGTTAATGTCATGATAATAATGGTT
TCTTAGACGTCAGGTGGCACTTTTCGGGGAAATGTGCGCGGAACCCCTATTTGTTTATTTTT
CTAAATACATTCAAATATGTATCCGCTCATGAGACAATAACCCTGATAAATGCTTCAATAAT
ATTGAAAAAGGAAGAGTATGAGTATTCAACATTTCCGTGTCGCCCTTATTCCCTTTTTTGCG
GCATTTTGCCTTCCTGTTTTTGCTCACCCAGAAACGCTGGTGAAAGTAAAAGATGCTGAAGA
TCAGTTGGGTGCACGAGTGGGTTACATCGAACTGGATCTCAACAGCGGTAAGATCCTTGAGA
GTTTTCGCCCCGAAGAACGTTTTCCAATGATGAGCACTTTTAAAGTTCTGCTATGTGGCGCG
GTATTATCCCGTATTGACGCCGGGCAAGAGCAACTCGGTCGCCGCATACACTATTCTCAGAA
TGACTTGGTTGAGTACTCACCAGTCACAGAAAAGCATCTTACGGATGGCATGACAGTAAGAG
AATTATGCAGTGCTGCCATAACCATGAGTGATAACACTGCGGCCAACTTACTTCTGACAACG
ATCGGAGGACCGAAGGAGCTAACCGCTTTTTTGCACAACATGGGGGATCATGTAACTCGCCT
TGATCGTTGGGAACCGGAGCTGAATGAAGCCATACCAAACGACGAGCGTGACACCA

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 68. An exemplary nucleotide sequence for vector c81.2 encoding mouse GJB2 is set forth in SEQ ID NO: 68 (hGJB2 GRE2 underlined; mouse GJB2 coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTC
AGTGATGCCTGAAACCTCAGATGGTACTGAACCCTCTATATAATCTGTTTTTTCCTATACAT
ATAACAATGTATAACAATATATTGTAATATAAGTTTTTGGATGCAGTCTCTCTCTCAAAATG
CTATCATATTTTCCAACTGTGGTTGACTACAGGTAACTGGAACCACAAAAATGAAACAGTGG
ATAAGAGGGCGACTCCTGTACCAAAGAAAAAAATAGAGTGTTGCAGCTGTAACATAGTTGAA
TGACTGAGTTAGACTGCATAACTGACACACAAAACCACATAAATATAAATGAAGGAATCTCT
GGGTGTAATCTGGTGCAAAGGTGACTGTGTTAATCATTAATCCACAAGTTGCTATCCTGAAG
TGTGCCAAATGCTTTATGTTTATTTCATCACATAGCTCTATAAAGAAAGGATTTGTAATTCC
TTTCTACAGAAGTGGAAAGTAAGTCTTAAGACTCAAAAAACTTTAAAAACTACAATGAAGTA
ACAACTTTTATTAATTTATTTTGTGTCTTTCCAGAATTTCTATATATATAGGAATGTGATAT
GAATCTATATGTGAATTGAATCTACATGAATATTGATGACTTTTATTTCCCCTTTTGCACAT
AAGATAGAATATTTTACCTACTATTCCACACTTTGCTTTTCTTAACATATCATGGGATCTTT
TTATATAAGTGAACAAAGAGTTTCTTCATTCTTTCACACAGTTTAATTAAGACCTCGAAGGG
GACTTGGGGGGTTCGGGGCTTTCGGGGGCGGTCGGGGGTTCGCGGACCCGGGAAGCTCTGAG
GACCCAGAGGCCGGGCGCGCTCCGCCCGCGGCGCCGCCCCCTCCGTAACTTTCCCAGTCTCC
GAGGGAAGAGGCGGGGTGTGGGGTGCGGTTAAAAGGCGCCACGGCGGGAGACAGGTGTTGCG
GCCCCGCAGCGCCCGCGCGCTCCTCTCCCCGACTCGGAGCCCCTCGGCGGCGCCCGGCCCAG
GACCCGCCTAGGAGCGCAGGAGCCCCAGCGCAGAGACCCCAACGCCGAGACCCCCGCCCCGG
CCCCGCCGCGCTTCCTCCCGACGCAGAGCAAACCGCCCAGAGTAGAAGCCATGGATTGGGGC
ACACTCCAGAGCATCCTCGGGGGTGTCAACAAACACTCCACCAGCATTGGAAAGATCTGGCT
CACGGTCCTCTTCATCTTCCGCATCATGATCCTCGTGGTGGCTGCAAAGGAGGTGTGGGGAG
ATGAGCAAGCCGATTTTGTCTGCAACACGCTCCAGCCTGGCTGCAAGAATGTATGCTACGAC
CACCACTTCCCCATCTCTCACATCCGGCTCTGGGCTCTGCAGCTGATCATGGTGTCCACGCC
AGCCCTCCTGGTAGCTATGCATGTGGCCTACCGGAGACATGAAAAGAAACGGAAGTTCATGA
AGGGAGAGATAAAGAACGAGTTTAAGGACATCGAAGAGATCAAAACCCAGAAGGTCCGTATC
GAAGGGTCCCTGTGGTGGACCTACACCACCAGCATCTTCTTCCGGGTCATCTTTGAAGCCGT
CTTCATGTACGTCTTTTACATCATGTACAATGGCTTCTTCATGCAACGTCTGGTGAAATGCA
ACGCTTGGCCCTGCCCCAATACAGTGGACTGCTTCATTTCCAGGCCCACAGAAAAGACTGTC
TTCACCGTGTTTATGATTTCTGTGTCTGGAATTTGCATTCTGCTAAATATCACAGAGCTGTG
CTATTTGTTCGTTAGGTATTGCTCAGGAAAGTCCAAAAGACCAGTCTACCCATACGATGTTC
CAGATTACGCTTAAAGGCGCGCCACCCCTGCAGGGAATTCCGCATTGCCCAGTTGTTAGATT
AAGAAATAGACAGCATGAGAGGGATGAGGCAACCCGTGCTCAGCTGTCAAGGCTCAGTCGCT
AGCATTTCCCAACACAAAGATTCTGACCTTAAATGCAACCATTTGAAACCCCTGTAGGCCTC
AGGTGAAACTCCAGATGCCACAATGGAGCTCTGCTCCCCTAAAGCCTCAAAACAAAGGCCTA
ATTCTATGCCTGTCTTAATTTTCTTTCACTTAAGTTAGTTCCACTGAGACCCCAGGCTGTTA
GGGGTTATTGGTGTAAGGTACTTTCATATTTTAAACAGAGGATATCGGCATTTGTTTCTTTC
TCTGAGGACAAGAGAAAAAAGCCAGGTTCCACAGAGGACACAGAGAAGGTTTGGGTGTCCTC
CTGGGGTTCTTTTTGCCAACTTTCCCCACGTTAAAGGTGAACATTGGTTCTTTCATTTGCTT
TGGAAGTTTTAATCTCTAACAGTGGACAAAGTTACCAGTGCCTTAAACTCTGTTACACTTTT
TGGAAGTGAAAACTTTGTAGTATGATAGGTTATTTTGATGTAAAGATGTTCTGGATACCATT
ATATGTTCCCCCTGTTTCAGAGGCTCAGATTGTAATATGTAAATGGTATGTCATTCGCTACT
ATGATTTAATTTGAAATATGGTCTTTTGGTTATGAATACTTTGCAGCACAGCTGAGAGGCTG
TCTGTTGTATTCATTGTGGTCATAGCACCTAACAACATTGTAGCCTCAATCGAGTGAGACAG
ACTAGAAGTTCCTAGTGATGGCTTATGATAGCAAATGGCCTCATGTCAAATATTTAGATGTA
ATTTTGTGTAAGAAATACAGACTGGATGTACCACCAACTACTACCTGTAATGACAGGCCTGT
CCAACACATCTCCCTTTTCCATGACTGTGGTAGCCAGCATCGGAAAGAACGCTGATTTAAAG
AGGTCGCTTGGGAATTTTATTGACACAGTACCATTTAATGGGGAGGACAAAATGGGGCAGGG
GAGGGAGAAGTTTCTGTCGTTAAAAACAGATTTGGAAAGACTGGACTCTAAAGTCTGTTGAT
TAAAGATGAGCTTTGTCTACTTCAAAAGTTTGTTTGCTTACCCCTTCAGCCTCCAATTTTTT
AAGTGAAAATATAGCTAATAACATGTGAAAAGAATAGAAGCTAAGGTTTAGATAAATATTGA
GCAGATCTATAGGAAGATTGAACCTGAATATTGCCATTATGCTTGACATGGTTTCCAAAAAA
AGCATTTTGTAATAATAAAGAATAGCTTTAATGATATGCTTGTAACTAAAATAATTTTGTAA
TGTATCAAATACATTTAAAACATTAAAATATAATCTCTATAATAATTTAAAATCTAATATGG
TTTTAATAGAACAGCGATATCAAGCTTATCGATAATCAACCTCTGGATTACAAAATTTGTGA
AAGATTGACTGGTATTCTTAACTATGTTGCTCCTTTTACGCTATGTGGATACGCTGCTTTAA
TGCCTTTGTATCATGCTATTGCTTCCCGTATGGCTTTCATTTTCTCCTCCTTGTATAAATCC
TGTGTTTGCTGACGCAACCCCCACTGGTTGGGGCATTGCCACCACCTGTCAGCTCCTTTCCG
GGACTTTCGCTTTCCCCCTCCCTATTGCCACGGCGGAACTCATCGCCGCCTGCCTTGCCCGC
TGCTGGACAGGGGCTCGGCTGTTGGGCACTGACAATTCCGTGGTGTTGTCGGGGAAATCATC
GTCCTTTCCTTGGCTGCTCGCCTATGTTGCCACCTGGATTCTGCGCGGGACGTCCTTCTGCT
ACGTCCCTTCGGCCCTCAATCCAGCGGACCTTCCTTCCCGCGGCCTGCTGCCGGCTCTGCGG
CCTCTTCCGCGTCTTCGCCTTCGCCCTCAGACGAGTCGGATCTCCCTTTGGGCCGCCTCCCC
GCGAATTCATCGATACCGAGCGCTGCTCGAGAGATCTGTGATAGCGGCCATCAAGCTGGCTG
TGCCTTCTAGTTGCCAGCCATCTGTTGTTTGCCCCTCCCCCGTGCCTTCCTTGACCCTGGAA
GGTGCCACTCCCACTGTCCTTTCCTAATAAAATGAGGAAATTGCATCGCATTGTCTGAGTAG
GTGTCATTCTATTCTGGGGGGTGGGGTGGGGCAGGACAGCAAGGGGGAGGATTGGGAAGACA
ATAGCAGGCATGCTGGGGACACGTGCGGACCGAGCGGCCGCAGGAACCCCTAGTGATGGAGT
TGGCCACTCCCTCTCTGCGCGCTCGCTCGCTCACTGAGGCCGGGCGACCAAAGGTCGCCCGA
CGCCCGGGCTTTGCCCGGGCGGCCTCAGTGAGCGAGCGAGCGCGCAGCTGCCTGCAGGGGCG
CCTGATGCGGTATTTTCTCCTTACGCATCTGTGCGGTATTTCACACCGCATACGTCAAAGCA
ACCATAGTACGCGCCCTGTAGCGGCGCATTAAGCGCGGCGGGTGTGGTGGTTACGCGCAGCG
TGACCGCTACACTTGCCAGCGCCCTAGCGCCCGCTCCTTTCGCTTTCTTCCCTTCCTTTCTC
GCCACGTTCGCCGGCTTTCCCCGTCAAGCTCTAAATCGGGGGCTCCCTTTAGGGTTCCGATT
TAGTGCTTTACGGCACCTCGACCCCAAAAAACTTGATTTGGGTGATGGTTCACGTAGTGGGC
CATCGCCCTGATAGACGGTTTTTCGCCCTTTGACGTTGGAGTCCACGTTCTTTAATAGTGGA
CTCTTGTTCCAAACTGGAACAACACTCAACCCTATCTCGGGCTATTCTTTTGATTTATAAGG
GATTTTGCCGATTTCGGCCTATTGGTTAAAAAATGAGCTGATTTAACAAAAATTTAACGCGA
ATTTTAACAAAATATTAACGTTTACAATTTTATGGTGCACTCTCAGTACAATCTGCTCTGAT
GCCGCATAGTTAAGCCAGCCCCGACACCCGCCAACACCCGCTGACGCGCCCTGACGGGCTTG
TCTGCTCCCGGCATCCGCTTACAGACAAGCTGTGACCGTCTCCGGGAGCTGCATGTGTCAGA
GGTTTTCACCGTCATCACCGAAACGCGCGAGACGAAAGGGCCTCGTGATACGCCTATTTTTA
TAGGTTAATGTCATGATAATAATGGTTTCTTAGACGTCAGGTGGCACTTTTCGGGGAAATGT
GCGCGGAACCCCTATTTGTTTATTTTTCTAAATACATTCAAATATGTATCCGCTCATGAGAC
AATAACCCTGATAAATGCTTCAATAATATTGAAAAAGGAAGAGTATGAGTATTCAACATTTC
CGTGTCGCCCTTATTCCCTTTTTTGCGGCATTTTGCCTTCCTGTTTTTGCTCACCCAGAAAC
GCTGGTGAAAGTAAAAGATGCTGAAGATCAGTTGGGTGCACGAGTGGGTTACATCGAACTGG
ATCTCAACAGCGGTAAGATCCTTGAGAGTTTTCGCCCCGAAGAACGTTTTCCAATGATGAGC
ACTTTTAAAGTTCTGCTATGTGGCGCGGTATTATCCCGTATTGACGCCGGGCAAGAGCAACT
CGGTCGCCGCATACACTATTCTCAGAATGACTTGGTTGAGTACTCACCAGTCACAGAAAAGC
ATCTTACGGATGGCATGACAGTAAGAGAATTATGCAGTGCTGCCATAACCATGAGTGATAAC
ACTGCGGCCAACTTACTTCTGACAACGATCGGAGGACCGAAGGAGCTAACCGCTTTTTTGCA
CAACATGGGGGATCATGTAACTCGCCTTGATCGTTGGGAACCGGAGCTGAATGAAGCCATAC
CAAACGACGAGCGTGACACCA

In some embodiments, an AAV vector described herein comprises an AAV 5′ ITR, a GJB2 GRE enhancer (hGJB2 GRE3), a GJB2 basal promoter, a GJB2 exon 1 5′ UTR, Kozak sequence, nucleotide sequence encoding a gene product (e.g., GJB2 or GFP), a GJB2 exon 2 3′ UTR, a WPRE, a bovine growth hormone poly A signal, and an AAV 3′ ITR (e.g., vector c.81.3).

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 49. An exemplary nucleotide sequence for vector c.81.3 is set forth in SEQ ID NO: 49 (hGJB2 GRE3 underlined; eGFP coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTG
CTATCTATCATCTTGAAGGGCTTCTGGAACAAGTTAGAATAGAGTCAACACTCATGAACTGC
TGTAGCAAAAAAAACTATAGATGTAGGATTGACAAGGGCAATAGAGCGATGACTCCCTGGCT
GTGTTGTATTTGATGGACGGCAGTAGCTTTTCACAAAATGCTCATTTGGATGTTTCAAATTA
AAACGTTTCACTTTCTAGAACCAATTACGTGGTCAGTTTAGCTCCTGAGGTCCCAGTCAGAG
GGGTATTCTGTAGCTTGCAAAGCCTCTCTTTGGGGACTGGACATGGAGTCTGTGGTCTTAGA
ATTCAGAACCGGGAGAATGTGTTAGCCACTCATCTAAGCTATTCCTTAAACGCTTTCAGAGC
CATCTCCACTGTGGGGAAAGAAGTTCTTTGTGTTCTCTGACTTAGTCTCATTCTAAAAAAAA
AAAAAAAAAAAAAAAAAAAGCAATTGCAATACCCAGAGCGCACAGTAGATGGCACTGAGACT
TGTCGGAAAGCTGGACGCACTCAAGAGGTGGCAGAAAAATCTATAGGTAAGCTTTTCTTCTA
GTCTGGTGTTGCTGCTCCTGACCTTATTAATGGGCTGAGAAATAGATTTCTTTCCTTTCCTT
TTCTTTTTTATATGAAATTAAATGAAGTATAAAAGAATATGAGAATGTGTTGCTATTAGCAA
GGATAAGTAATGCTTTAGGAAACGTTTGGTTCATGTGTGTGTTTTCAGACTGATGTGTGTCC
TGGATCCAGTGTAAAATGTACTTCTGTCTGTAGGTCTCTGCCACAGAAAAGTTGGAAAGCCA
TTGTTGTATTCCATTTCCAGGGCAACAAAAGATACCACTGTCACTTCATGTGAAATGGTGTT
GTTTAATTAAGACCTCGAAGGGGACTTGGGGGGTTCGGGGCTTTCGGGGGCGGTCGGGGGTT
CGCGGACCCGGGAAGCTCTGAGGACCCAGAGGCCGGGCGCGCTCCGCCCGCGGCGCCGCCCC
CTCCGTAACTTTCCCAGTCTCCGAGGGAAGAGGCGGGGTGTGGGGTGCGGTTAAAAGGCGCC
ACGGCGGGAGACAGGTGTTGCGGCCCCGCAGCGCCCGCGCGCTCCTCTCCCCGACTCGGAGC
CCCTCGGCGGCGCCCGGCCCAGGACCCGCCTAGGAGCGCAGGAGCCCCAGCGCAGAGACCCC
AACGCCGAGACCCCCGCCCCGGCCCCGCCGCGCTTCCTCCCGACGCAGAGCAAACCGCCCAG
AGTAGAAGCCATGGTGAGCAAGGGCGAGGAGCTGTTCACCGGGGTGGTGCCCATCCTGGTCG
AGCTGGACGGCGACGTAAACGGCCACAAGTTCAGCGTGTCCGGCGAGGGCGAGGGCGATGCC
ACCTACGGCAAGCTGACCCTGAAGTTCATCTGCACCACCGGCAAGCTGCCCGTGCCCTGGCC
CACCCTCGTGACCACCCTGACCTACGGCGTGCAGTGCTTCAGCCGCTACCCCGACCACATGA
AGCAGCACGACTTCTTCAAGTCCGCCATGCCCGAAGGCTACGTCCAGGAGCGCACCATCTTC
TTCAAGGACGACGGCAACTACAAGACCCGCGCCGAGGTGAAGTTCGAGGGCGACACCCTGGT
GAACCGCATCGAGCTGAAGGGCATCGACTTCAAGGAGGACGGCAACATCCTGGGGCACAAGC
TGGAGTACAACTACAACAGCCACAACGTCTATATCATGGCCGACAAGCAGAAGAACGGCATC
AAGGTGAACTTCAAGATCCGCCACAACATCGAGGACGGCAGCGTGCAGCTCGCCGACCACTA
CCAGCAGAACACCCCCATCGGCGACGGCCCCGTGCTGCTGCCCGACAACCACTACCTGAGCA
CCCAGTCCGCCCTGAGCAAAGACCCCAACGAGAAGCGCGATCACATGGTCCTGCTGGAGTTC
GTGACCGCCGCCGGGATCACTCTCGGCATGGACGAGCTGTACAAGTAAAGGCGCGCCACCCC
TGCAGGGAATTCCGCATTGCCCAGTTGTTAGATTAAGAAATAGACAGCATGAGAGGGATGAG
GCAACCCGTGCTCAGCTGTCAAGGCTCAGTCGCTAGCATTTCCCAACACAAAGATTCTGACC
CTCTGCTCCCCTAAAGCCTCAAAACAAAGGCCTAATTCTATGCCTGTCTTAATTTTCTTTCA
CTTAAGTTAGTTCCACTGAGACCCCAGGCTGTTAGGGGTTATTGGTGTAAGGTACTTTCATA
TTTTAAACAGAGGATATCGGCATTTGTTTCTTTCTCTGAGGACAAGAGAAAAAAGCCAGGTT
CCACAGAGGACACAGAGAAGGTTTGGGTGTCCTCCTGGGGTTCTTTTTGCCAACTTTCCCCA
CGTTAAAGGTGAACATTGGTTCTTTCATTTGCTTTGGAAGTTTTAATCTCTAACAGTGGACA
AAGTTACCAGTGCCTTAAACTCTGTTACACTTTTTGGAAGTGAAAACTTTGTAGTATGATAG
GTTATTTTGATGTAAAGATGTTCTGGATACCATTATATGTTCCCCCTGTTTCAGAGGCTCAG
ATTGTAATATGTAAATGGTATGTCATTCGCTACTATGATTTAATTTGAAATATGGTCTTTTG
GTTATGAATACTTTGCAGCACAGCTGAGAGGCTGTCTGTTGTATTCATTGTGGTCATAGCAC
CTAACAACATTGTAGCCTCAATCGAGTGAGACAGACTAGAAGTTCCTAGTGATGGCTTATGA
TAGCAAATGGCCTCATGTCAAATATTTAGATGTAATTTTGTGTAAGAAATACAGACTGGATG
TACCACCAACTACTACCTGTAATGACAGGCCTGTCCAACACATCTCCCTTTTCCATGACTGT
GGTAGCCAGCATCGGAAAGAACGCTGATTTAAAGAGGTCGCTTGGGAATTTTATTGACACAG
TACCATTTAATGGGGAGGACAAAATGGGGCAGGGGAGGGAGAAGTTTCTGTCGTTAAAAACA
GATTTGGAAAGACTGGACTCTAAAGTCTGTTGATTAAAGATGAGCTTTGTCTACTTCAAAAG
TTTGTTTGCTTACCCCTTCAGCCTCCAATTTTTTAAGTGAAAATATAGCTAATAACATGTGA
AAAGAATAGAAGCTAAGGTTTAGATAAATATTGAGCAGATCTATAGGAAGATTGAACCTGAA
TATTGCCATTATGCTTGACATGGTTTCCAAAAAATGGTACTCCACATATTTCAGTGAGGGTA
AGTATTTTCCTGTTGTCAAGAATAGCATTGTAAAAGCATTTTGTAATAATAAAGAATAGCTT
TAATGATATGCTTGTAACTAAAATAATTTTGTAATGTATCAAATACATTTAAAACATTAAAA
TATAATCTCTATAATAATTTAAAATCTAATATGGTTTTAATAGAACAGCGATATCAAGCTTA
TCGATAATCAACCTCTGGATTACAAAATTTGTGAAAGATTGACTGGTATTCTTAACTATGTT
GCTCCTTTTACGCTATGTGGATACGCTGCTTTAATGCCTTTGTATCATGCTATTGCTTCCCG
GGCCCGTTGTCAGGCAACGTGGCGTGGTGTGCACTGTGTTTGCTGACGCAACCCCCACTGGT
TGGGGCATTGCCACCACCTGTCAGCTCCTTTCCGGGACTTTCGCTTTCCCCCTCCCTATTGC
CACGGCGGAACTCATCGCCGCCTGCCTTGCCCGCTGCTGGACAGGGGCTCGGCTGTTGGGCA
CTGACAATTCCGTGGTGTTGTCGGGGAAATCATCGTCCTTTCCTTGGCTGCTCGCCTATGTT
GCCACCTGGATTCTGCGCGGGACGTCCTTCTGCTACGTCCCTTCGGCCCTCAATCCAGCGGA
CCTTCCTTCCCGCGGCCTGCTGCCGGCTCTGCGGCCTCTTCCGCGTCTTCGCCTTCGCCCTC
AGACGAGTCGGATCTCCCTTTGGGCCGCCTCCCCGCGAATTCATCGATACCGAGCGCTGCTC
GAGAGATCTGTGATAGCGGCCATCAAGCTGGCTGTGCCTTCTAGTTGCCAGCCATCTGTTGT
TTGCCCCTCCCCCGTGCCTTCCTTGACCCTGGAAGGTGCCACTCCCACTGTCCTTTCCTAAT
AAAATGAGGAAATTGCATCGCATTGTCTGAGTAGGTGTCATTCTATTCTGGGGGGTGGGGTG
GGGCAGGACAGCAAGGGGGAGGATTGGGAAGACAATAGCAGGCATGCTGGGGACACGTGCGG
ACCGAGCGGCCGCAGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCCCGGGCGGCCTCAG
TGAGCGAGCGAGCGCGCAGCTGCCTGCAGGGGCGCCTGATGCGGTATTTTCTCCTTACGCAT
CTGTGCGGTATTTCACACCGCATACGTCAAAGCAACCATAGTACGCGCCCTGTAGCGGCGCA
TTAAGCGCGGCGGGTGTGGTGGTTACGCGCAGCGTGACCGCTACACTTGCCAGCGCCCTAGC
GCCCGCTCCTTTCGCTTTCTTCCCTTCCTTTCTCGCCACGTTCGCCGGCTTTCCCCGTCAAG
CTCTAAATCGGGGGCTCCCTTTAGGGTTCCGATTTAGTGCTTTACGGCACCTCGACCCCAAA
AAACTTGATTTGGGTGATGGTTCACGTAGTGGGCCATCGCCCTGATAGACGGTTTTTCGCCC
TTTGACGTTGGAGTCCACGTTCTTTAATAGTGGACTCTTGTTCCAAACTGGAACAACACTCA
ACCCTATCTCGGGCTATTCTTTTGATTTATAAGGGATTTTGCCGATTTCGGCCTATTGGTTA
AAAAATGAGCTGATTTAACAAAAATTTAACGCGAATTTTAACAAAATATTAACGTTTACAAT
TTTATGGTGCACTCTCAGTACAATCTGCTCTGATGCCGCATAGTTAAGCCAGCCCCGACACC
CGCCAACACCCGCTGACGCGCCCTGACGGGCTTGTCTGCTCCCGGCATCCGCTTACAGACAA
GCTGTGACCGTCTCCGGGAGCTGCATGTGTCAGAGGTTTTCACCGTCATCACCGAAACGCGC
GAGACGAAAGGGCCTCGTGATACGCCTATTTTTATAGGTTAATGTCATGATAATAATGGTTT
CTTAGACGTCAGGTGGCACTTTTCGGGGAAATGTGCGCGGAACCCCTATTTGTTTATTTTTC
TAAATACATTCAAATATGTATCCGCTCATGAGACAATAACCCTGATAAATGCTTCAATAATA
TTGAAAAAGGAAGAGTATGAGTATTCAACATTTCCGTGTCGCCCTTATTCCCTTTTTTGCGG
CATTTTGCCTTCCTGTTTTTGCTCACCCAGAAACGCTGGTGAAAGTAAAAGATGCTGAAGAT
CAGTTGGGTGCACGAGTGGGTTACATCGAACTGGATCTCAACAGCGGTAAGATCCTTGAGAG
TTTTCGCCCCGAAGAACGTTTTCCAATGATGAGCACTTTTAAAGTTCTGCTATGTGGCGCGG
TATTATCCCGTATTGACGCCGGGCAAGAGCAACTCGGTCGCCGCATACACTATTCTCAGAAT
GACTTGGTTGAGTACTCACCAGTCACAGAAAAGCATCTTACGGATGGCATGACAGTAAGAGA
ATTATGCAGTGCTGCCATAACCATGAGTGATAACACTGCGGCCAACTTACTTCTGACAACGA
TCGGAGGACCGAAGGAGCTAACCGCTTTTTTGCACAACATGGGGGATCATGTAACTCGCCTT
GATCGTTGGGAACCGGAGCTGAATGAAGCCATACCAAACGACGAGCGTGACACCA

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 70. An exemplary nucleotide sequence for vector c.81.3 is set forth in SEQ ID NO: 70 (hGJB2 GRE3 underlined; human GJB2 coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTG
CTATCTATCATCTTGAAGGGCTTCTGGAACAAGTTAGAATAGAGTCAACACTCATGAACTGC
TGTAGCAAAAAAAACTATAGATGTAGGATTGACAAGGGCAATAGAGCGATGACTCCCTGGCT
GTGTTGTATTTGATGGACGGCAGTAGCTTTTCACAAAATGCTCATTTGGATGTTTCAAATTA
AAACGTTTCACTTTCTAGAACCAATTACGTGGTCAGTTTAGCTCCTGAGGTCCCAGTCAGAG
GGGTATTCTGTAGCTTGCAAAGCCTCTCTTTGGGGACTGGACATGGAGTCTGTGGTCTTAGA
ATTCAGAACCGGGAGAATGTGTTAGCCACTCATCTAAGCTATTCCTTAAACGCTTTCAGAGC
CATCTCCACTGTGGGGAAAGAAGTTCTTTGTGTTCTCTGACTTAGTCTCATTCTAAAAAAAA
AAAAAAAAAAAAAAAAAAAGCAATTGCAATACCCAGAGCGCACAGTAGATGGCACTGAGACT
TGTCGGAAAGCTGGACGCACTCAAGAGGTGGCAGAAAAATCTATAGGTAAGCTTTTCTTCTA
GTCTGGTGTTGCTGCTCCTGACCTTATTAATGGGCTGAGAAATAGATTTCTTTCCTTTCCTT
TTCTTTTTTATATGAAATTAAATGAAGTATAAAAGAATATGAGAATGTGTTGCTATTAGCAA
GGATAAGTAATGCTTTAGGAAACGTTTGGTTCATGTGTGTGTTTTCAGACTGATGTGTGTCC
TGGATCCAGTGTAAAATGTACTTCTGTCTGTAGGTCTCTGCCACAGAAAAGTTGGAAAGCCA
TTGTTGTATTCCATTTCCAGGGCAACAAAAGATACCACTGTCACTTCATGTGAAATGGTGTT
GTTTAATTAAGACCTCGAAGGGGACTTGGGGGGTTCGGGGCTTTCGGGGGCGGTCGGGGGTT
CGCGGACCCGGGAAGCTCTGAGGACCCAGAGGCCGGGCGCGCTCCGCCCGCGGCGCCGCCCC
CTCCGTAACTTTCCCAGTCTCCGAGGGAAGAGGCGGGGTGTGGGGTGCGGTTAAAAGGCGCC
ACGGCGGGAGACAGGTGTTGCGGCCCCGCAGCGCCCGCGCGCTCCTCTCCCCGACTCGGAGC
CCCTCGGCGGCGCCCGGCCCAGGACCCGCCTAGGAGCGCAGGAGCCCCAGCGCAGAGACCCC
AACGCCGAGACCCCCGCCCCGGCCCCGCCGCGCTTCCTCCCGACGCAGAGCAAACCGCCCAG
AGTAGAAGCCATGGATTGGGGCACGCTGCAGACGATCCTGGGGGGTGTGAACAAACACTCCA
CCAGCATTGGAAAGATCTGGCTCACCGTCCTCTTCATTTTTCGCATTATGATCCTCGTTGTG
GCTGCAAAGGAGGTGTGGGGAGATGAGCAGGCCGACTTTGTCTGCAACACCCTGCAGCCAGG
CTGCAAGAACGTGTGCTACGATCACTACTTCCCCATCTCCCACATCCGGCTATGGGCCCTGC
AGCTGATCTTCGTGTCCACGCCAGCGCTCCTAGTGGCCATGCACGTGGCCTACCGGAGACAT
GAGAAGAAGAGGAAGTTCATCAAGGGGGAGATAAAGAGTGAATTTAAGGACATCGAGGAGAT
CAAAACCCAGAAGGTCCGCATCGAAGGCTCCCTGTGGTGGACCTACACAAGCAGCATCTTCT
TCCGGGTCATCTTCGAAGCCGCCTTCATGTACGTCTTCTATGTCATGTACGACGGCTTCTCC
ATGCAGCGGCTGGTGAAGTGCAACGCCTGGCCTTGTCCCAACACTGTGGACTGCTTTGTGTC
CCGGCCCACGGAGAAGACTGTCTTCACAGTGTTCATGATTGCAGTGTCTGGAATTTGCATCC
TGCTGAATGTCACTGAATTGTGTTATTTGCTAATTAGATATTGTTCTGGGAAGTCAAAAAAG
CCAGTTTAAAGGCGCGCCACCCCTGCAGGGAATTCCGCATTGCCCAGTTGTTAGATTAAGAA
ATAGACAGCATGAGAGGGATGAGGCAACCCGTGCTCAGCTGTCAAGGCTCAGTCGCTAGCAT
TTCCCAACACAAAGATTCTGACCTTAAATGCAACCATTTGAAACCCCTGTAGGCCTCAGGTG
AAACTCCAGATGCCACAATGGAGCTCTGCTCCCCTAAAGCCTCAAAACAAAGGCCTAATTCT
ATGCCTGTCTTAATTTTCTTTCACTTAAGTTAGTTCCACTGAGACCCCAGGCTGTTAGGGGT
TATTGGTGTAAGGTACTTTCATATTTTAAACAGAGGATATCGGCATTTGTTTCTTTCTCTGA
GGACAAGAGAAAAAAGCCAGGTTCCACAGAGGACACAGAGAAGGTTTGGGTGTCCTCCTGGG
GTTCTTTTTGCCAACTTTCCCCACGTTAAAGGTGAACATTGGTTCTTTCATTTGCTTTGGAA
GTTTTAATCTCTAACAGTGGACAAAGTTACCAGTGCCTTAAACTCTGTTACACTTTTTGGAA
GTGAAAACTTTGTAGTATGATAGGTTATTTTGATGTAAAGATGTTCTGGATACCATTATATG
TTCCCCCTGTTTCAGAGGCTCAGATTGTAATATGTAAATGGTATGTCATTCGCTACTATGAT
TTAATTTGAAATATGGTCTTTTGGTTATGAATACTTTGCAGCACAGCTGAGAGGCTGTCTGT
TGTATTCATTGTGGTCATAGCACCTAACAACATTGTAGCCTCAATCGAGTGAGACAGACTAG
AAGTTCCTAGTGATGGCTTATGATAGCAAATGGCCTCATGTCAAATATTTAGATGTAATTTT
GTGTAAGAAATACAGACTGGATGTACCACCAACTACTACCTGTAATGACAGGCCTGTCCAAC
ACATCTCCCTTTTCCATGACTGTGGTAGCCAGCATCGGAAAGAACGCTGATTTAAAGAGGTC
GCTTGGGAATTTTATTGACACAGTACCATTTAATGGGGAGGACAAAATGGGGCAGGGGAGGG
AGAAGTTTCTGTCGTTAAAAACAGATTTGGAAAGACTGGACTCTAAAGTCTGTTGATTAAAG
AAAATATAGCTAATAACATGTGAAAAGAATAGAAGCTAAGGTTTAGATAAATATTGAGCAGA
TCTATAGGAAGATTGAACCTGAATATTGCCATTATGCTTGACATGGTTTCCAAAAAATGGTA
TTTGTAATAATAAAGAATAGCTTTAATGATATGCTTGTAACTAAAATAATTTTGTAATGTAT
CAAATACATTTAAAACATTAAAATATAATCTCTATAATAATTTAAAATCTAATATGGTTTTA
ATAGAACAGCGATATCAAGCTTATCGATAATCAACCTCTGGATTACAAAATTTGTGAAAGAT
TGACTGGTATTCTTAACTATGTTGCTCCTTTTACGCTATGTGGATACGCTGCTTTAATGCCT
TTGTATCATGCTATTGCTTCCCGTATGGCTTTCATTTTCTCCTCCTTGTATAAATCCTGGTT
GCTGTCTCTTTATGAGGAGTTGTGGCCCGTTGTCAGGCAACGTGGCGTGGTGTGCACTGTGT
TTGCTGACGCAACCCCCACTGGTTGGGGCATTGCCACCACCTGTCAGCTCCTTTCCGGGACT
TTCGCTTTCCCCCTCCCTATTGCCACGGCGGAACTCATCGCCGCCTGCCTTGCCCGCTGCTG
GACAGGGGCTCGGCTGTTGGGCACTGACAATTCCGTGGTGTTGTCGGGGAAATCATCGTCCT
TTCCTTGGCTGCTCGCCTATGTTGCCACCTGGATTCTGCGCGGGACGTCCTTCTGCTACGTC
CCTTCGGCCCTCAATCCAGCGGACCTTCCTTCCCGCGGCCTGCTGCCGGCTCTGCGGCCTCT
TCCGCGTCTTCGCCTTCGCCCTCAGACGAGTCGGATCTCCCTTTGGGCCGCCTCCCCGCGAA
TTCATCGATACCGAGCGCTGCTCGAGAGATCTGTGATAGCGGCCATCAAGCTGGCTGTGCCT
TCTAGTTGCCAGCCATCTGTTGTTTGCCCCTCCCCCGTGCCTTCCTTGACCCTGGAAGGTGC
CACTCCCACTGTCCTTTCCTAATAAAATGAGGAAATTGCATCGCATTGTCTGAGTAGGTGTC
ATTCTATTCTGGGGGGTGGGGTGGGGCAGGACAGCAAGGGGGAGGATTGGGAAGACAATAGC
AGGCATGCTGGGGACACGTGCGGACCGAGCGGCCGCAGGAACCCCTAGTGATGGAGTTGGCC
ACTCCCTCTCTGCGCGCTCGCTCGCTCACTGAGGCCGGGCGACCAAAGGTCGCCCGACGCCC
GGGCTTTGCCCGGGCGGCCTCAGTGAGCGAGCGAGCGCGCAGCTGCCTGCAGGGGCGCCTGA
TGCGGTATTTTCTCCTTACGCATCTGTGCGGTATTTCACACCGCATACGTCAAAGCAACCAT
AGTACGCGCCCTGTAGCGGCGCATTAAGCGCGGCGGGTGTGGTGGTTACGCGCAGCGTGACC
GCTACACTTGCCAGCGCCCTAGCGCCCGCTCCTTTCGCTTTCTTCCCTTCCTTTCTCGCCAC
GTTCGCCGGCTTTCCCCGTCAAGCTCTAAATCGGGGGCTCCCTTTAGGGTTCCGATTTAGTG
CTTTACGGCACCTCGACCCCAAAAAACTTGATTTGGGTGATGGTTCACGTAGTGGGCCATCG
CCCTGATAGACGGTTTTTCGCCCTTTGACGTTGGAGTCCACGTTCTTTAATAGTGGACTCTT
GTTCCAAACTGGAACAACACTCAACCCTATCTCGGGCTATTCTTTTGATTTATAAGGGATTT
TGCCGATTTCGGCCTATTGGTTAAAAAATGAGCTGATTTAACAAAAATTTAACGCGAATTTT
AACAAAATATTAACGTTTACAATTTTATGGTGCACTCTCAGTACAATCTGCTCTGATGCCGC
ATAGTTAAGCCAGCCCCGACACCCGCCAACACCCGCTGACGCGCCCTGACGGGCTTGTCTGC
TCCCGGCATCCGCTTACAGACAAGCTGTGACCGTCTCCGGGAGCTGCATGTGTCAGAGGTTT
TCACCGTCATCACCGAAACGCGCGAGACGAAAGGGCCTCGTGATACGCCTATTTTTATAGGT
TAATGTCATGATAATAATGGTTTCTTAGACGTCAGGTGGCACTTTTCGGGGAAATGTGCGCG
GAACCCCTATTTGTTTATTTTTCTAAATACATTCAAATATGTATCCGCTCATGAGACAATAA
CCCTGATAAATGCTTCAATAATATTGAAAAAGGAAGAGTATGAGTATTCAACATTTCCGTGT
CGCCCTTATTCCCTTTTTTGCGGCATTTTGCCTTCCTGTTTTTGCTCACCCAGAAACGCTGG
TGAAAGTAAAAGATGCTGAAGATCAGTTGGGTGCACGAGTGGGTTACATCGAACTGGATCTC
AACAGCGGTAAGATCCTTGAGAGTTTTCGCCCCGAAGAACGTTTTCCAATGATGAGCACTTT
TAAAGTTCTGCTATGTGGCGCGGTATTATCCCGTATTGACGCCGGGCAAGAGCAACTCGGTC
GCCGCATACACTATTCTCAGAATGACTTGGTTGAGTACTCACCAGTCACAGAAAAGCATCTT
ACGGATGGCATGACAGTAAGAGAATTATGCAGTGCTGCCATAACCATGAGTGATAACACTGC
GGCCAACTTACTTCTGACAACGATCGGAGGACCGAAGGAGCTAACCGCTTTTTTGCACAACA
TGGGGGATCATGTAACTCGCCTTGATCGTTGGGAACCGGAGCTGAATGAAGCCATACCAAAC
GACGAGCGTGACACCA

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 71. An exemplary nucleotide sequence for vector c.81.3 is set forth in SEQ ID NO: 71 (hGJB2 GRE3 underlined; mouse GJB2 coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTG
CTATCTATCATCTTGAAGGGCTTCTGGAACAAGTTAGAATAGAGTCAACACTCATGAACTGC
TGTAGCAAAAAAAACTATAGATGTAGGATTGACAAGGGCAATAGAGCGATGACTCCCTGGCT
GTGTTGTATTTGATGGACGGCAGTAGCTTTTCACAAAATGCTCATTTGGATGTTTCAAATTA
AAACGTTTCACTTTCTAGAACCAATTACGTGGTCAGTTTAGCTCCTGAGGTCCCAGTCAGAG
GGGTATTCTGTAGCTTGCAAAGCCTCTCTTTGGGGACTGGACATGGAGTCTGTGGTCTTAGA
ATTCAGAACCGGGAGAATGTGTTAGCCACTCATCTAAGCTATTCCTTAAACGCTTTCAGAGC
CATCTCCACTGTGGGGAAAGAAGTTCTTTGTGTTCTCTGACTTAGTCTCATTCTAAAAAAAA
AAAAAAAAAAAAAAAAAAAGCAATTGCAATACCCAGAGCGCACAGTAGATGGCACTGAGACT
TGTCGGAAAGCTGGACGCACTCAAGAGGTGGCAGAAAAATCTATAGGTAAGCTTTTCTTCTA
GTCTGGTGTTGCTGCTCCTGACCTTATTAATGGGCTGAGAAATAGATTTCTTTCCTTTCCTT
TTCTTTTTTATATGAAATTAAATGAAGTATAAAAGAATATGAGAATGTGTTGCTATTAGCAA
GGATAAGTAATGCTTTAGGAAACGTTTGGTTCATGTGTGTGTTTTCAGACTGATGTGTGTCC
TGGATCCAGTGTAAAATGTACTTCTGTCTGTAGGTCTCTGCCACAGAAAAGTTGGAAAGCCA
TTGTTGTATTCCATTTCCAGGGCAACAAAAGATACCACTGTCACTTCATGTGAAATGGTGTT
GTTTAATTAAGACCTCGAAGGGGACTTGGGGGGTTCGGGGCTTTCGGGGGCGGTCGGGGGTT
CGCGGACCCGGGAAGCTCTGAGGACCCAGAGGCCGGGCGCGCTCCGCCCGCGGCGCCGCCCC
CTCCGTAACTTTCCCAGTCTCCGAGGGAAGAGGCGGGGTGTGGGGTGCGGTTAAAAGGCGCC
ACGGCGGGAGACAGGTGTTGCGGCCCCGCAGCGCCCGCGCGCTCCTCTCCCCGACTCGGAGC
CCCTCGGCGGCGCCCGGCCCAGGACCCGCCTAGGAGCGCAGGAGCCCCAGCGCAGAGACCCC
AACGCCGAGACCCCCGCCCCGGCCCCGCCGCGCTTCCTCCCGACGCAGAGCAAACCGCCCAG
AGTAGAAGCCATGGATTGGGGCACACTCCAGAGCATCCTCGGGGGTGTCAACAAACACTCCA
CCAGCATTGGAAAGATCTGGCTCACGGTCCTCTTCATCTTCCGCATCATGATCCTCGTGGTG
GCTGCAAAGGAGGTGTGGGGAGATGAGCAAGCCGATTTTGTCTGCAACACGCTCCAGCCTGG
CTGCAAGAATGTATGCTACGACCACCACTTCCCCATCTCTCACATCCGGCTCTGGGCTCTGC
AGCTGATCATGGTGTCCACGCCAGCCCTCCTGGTAGCTATGCATGTGGCCTACCGGAGACAT
GAAAAGAAACGGAAGTTCATGAAGGGAGAGATAAAGAACGAGTTTAAGGACATCGAAGAGAT
CAAAACCCAGAAGGTCCGTATCGAAGGGTCCCTGTGGTGGACCTACACCACCAGCATCTTCT
TCCGGGTCATCTTTGAAGCCGTCTTCATGTACGTCTTTTACATCATGTACAATGGCTTCTTC
ATGCAACGTCTGGTGAAATGCAACGCTTGGCCCTGCCCCAATACAGTGGACTGCTTCATTTC
CAGGCCCACAGAAAAGACTGTCTTCACCGTGTTTATGATTTCTGTGTCTGGAATTTGCATTC
TGCTAAATATCACAGAGCTGTGCTATTTGTTCGTTAGGTATTGCTCAGGAAAGTCCAAAAGA
CCAGTCTACCCATACGATGTTCCAGATTACGCTTAAAGGCGCGCCACCCCTGCAGGGAATTC
CGCATTGCCCAGTTGTTAGATTAAGAAATAGACAGCATGAGAGGGATGAGGCAACCCGTGCT
CAGCTGTCAAGGCTCAGTCGCTAGCATTTCCCAACACAAAGATTCTGACCTTAAATGCAACC
ATTTGAAACCCCTGTAGGCCTCAGGTGAAACTCCAGATGCCACAATGGAGCTCTGCTCCCCT
AAAGCCTCAAAACAAAGGCCTAATTCTATGCCTGTCTTAATTTTCTTTCACTTAAGTTAGTT
CCACTGAGACCCCAGGCTGTTAGGGGTTATTGGTGTAAGGTACTTTCATATTTTAAACAGAG
GATATCGGCATTTGTTTCTTTCTCTGAGGACAAGAGAAAAAAGCCAGGTTCCACAGAGGACA
CAGAGAAGGTTTGGGTGTCCTCCTGGGGTTCTTTTTGCCAACTTTCCCCACGTTAAAGGTGA
ACATTGGTTCTTTCATTTGCTTTGGAAGTTTTAATCTCTAACAGTGGACAAAGTTACCAGTG
CCTTAAACTCTGTTACACTTTTTGGAAGTGAAAACTTTGTAGTATGATAGGTTATTTTGATG
TAAAGATGTTCTGGATACCATTATATGTTCCCCCTGTTTCAGAGGCTCAGATTGTAATATGT
AAATGGTATGTCATTCGCTACTATGATTTAATTTGAAATATGGTCTTTTGGTTATGAATACT
TTGCAGCACAGCTGAGAGGCTGTCTGTTGTATTCATTGTGGTCATAGCACCTAACAACATTG
TAGCCTCAATCGAGTGAGACAGACTAGAAGTTCCTAGTGATGGCTTATGATAGCAAATGGCC
TCATGTCAAATATTTAGATGTAATTTTGTGTAAGAAATACAGACTGGATGTACCACCAACTA
CTACCTGTAATGACAGGCCTGTCCAACACATCTCCCTTTTCCATGACTGTGGTAGCCAGCAT
CGGAAAGAACGCTGATTTAAAGAGGTCGCTTGGGAATTTTATTGACACAGTACCATTTAATG
GGGAGGACAAAATGGGGCAGGGGAGGGAGAAGTTTCTGTCGTTAAAAACAGATTTGGAAAGA
CTGGACTCTAAAGTCTGTTGATTAAAGATGAGCTTTGTCTACTTCAAAAGTTTGTTTGCTTA
CCCCTTCAGCCTCCAATTTTTTAAGTGAAAATATAGCTAATAACATGTGAAAAGAATAGAAG
CTAAGGTTTAGATAAATATTGAGCAGATCTATAGGAAGATTGAACCTGAATATTGCCATTAT
GCTTGACATGGTTTCCAAAAAATGGTACTCCACATATTTCAGTGAGGGTAAGTATTTTCCTG
TTGTCAAGAATAGCATTGTAAAAGCATTTTGTAATAATAAAGAATAGCTTTAATGATATGCT
TGTAACTAAAATAATTTTGTAATGTATCAAATACATTTAAAACATTAAAATATAATCTCTAT
AATAATTTAAAATCTAATATGGTTTTAATAGAACAGCGATATCAAGCTTATCGATAATCAAC
CTCTGGATTACAAAATTTGTGAAAGATTGACTGGTATTCTTAACTATGTTGCTCCTTTTACG
CTATGTGGATACGCTGCTTTAATGCCTTTGTATCATGCTATTGCTTCCCGTATGGCTTTCAT
GGCAACGTGGCGTGGTGTGCACTGTGTTTGCTGACGCAACCCCCACTGGTTGGGGCATTGCC
ACCACCTGTCAGCTCCTTTCCGGGACTTTCGCTTTCCCCCTCCCTATTGCCACGGCGGAACT
CATCGCCGCCTGCCTTGCCCGCTGCTGGACAGGGGCTCGGCTGTTGGGCACTGACAATTCCG
TGGTGTTGTCGGGGAAATCATCGTCCTTTCCTTGGCTGCTCGCCTATGTTGCCACCTGGATT
CTGCGCGGGACGTCCTTCTGCTACGTCCCTTCGGCCCTCAATCCAGCGGACCTTCCTTCCCG
CGGCCTGCTGCCGGCTCTGCGGCCTCTTCCGCGTCTTCGCCTTCGCCCTCAGACGAGTCGGA
TCTCCCTTTGGGCCGCCTCCCCGCGAATTCATCGATACCGAGCGCTGCTCGAGAGATCTGTG
ATAGCGGCCATCAAGCTGGCTGTGCCTTCTAGTTGCCAGCCATCTGTTGTTTGCCCCTCCCC
CGTGCCTTCCTTGACCCTGGAAGGTGCCACTCCCACTGTCCTTTCCTAATAAAATGAGGAAA
TTGCATCGCATTGTCTGAGTAGGTGTCATTCTATTCTGGGGGGTGGGGTGGGGCAGGACAGC
AAGGGGGAGGATTGGGAAGACAATAGCAGGCATGCTGGGGACACGTGCGGACCGAGCGGCCG
CAGGAACCCCTAGTGATGGAGTTGGCCACTCCCTCTCTGCGCGCTCGCTCGCTCACTGAGGC
CGGGCGACCAAAGGTCGCCCGACGCCCGGGCTTTGCCCGGGCGGCCTCAGTGAGCGAGCGAG
CGCGCAGCTGCCTGCAGGGGCGCCTGATGCGGTATTTTCTCCTTACGCATCTGTGCGGTATT
TCACACCGCATACGTCAAAGCAACCATAGTACGCGCCCTGTAGCGGCGCATTAAGCGCGGCG
GGTGTGGTGGTTACGCGCAGCGTGACCGCTACACTTGCCAGCGCCCTAGCGCCCGCTCCTTT
CGCTTTCTTCCCTTCCTTTCTCGCCACGTTCGCCGGCTTTCCCCGTCAAGCTCTAAATCGGG
GGCTCCCTTTAGGGTTCCGATTTAGTGCTTTACGGCACCTCGACCCCAAAAAACTTGATTTG
GGTGATGGTTCACGTAGTGGGCCATCGCCCTGATAGACGGTTTTTCGCCCTTTGACGTTGGA
GTCCACGTTCTTTAATAGTGGACTCTTGTTCCAAACTGGAACAACACTCAACCCTATCTCGG
GCTATTCTTTTGATTTATAAGGGATTTTGCCGATTTCGGCCTATTGGTTAAAAAATGAGCTG
ATTTAACAAAAATTTAACGCGAATTTTAACAAAATATTAACGTTTACAATTTTATGGTGCAC
TCTCAGTACAATCTGCTCTGATGCCGCATAGTTAAGCCAGCCCCGACACCCGCCAACACCCG
CTGACGCGCCCTGACGGGCTTGTCTGCTCCCGGCATCCGCTTACAGACAAGCTGTGACCGTC
TCCGGGAGCTGCATGTGTCAGAGGTTTTCACCGTCATCACCGAAACGCGCGAGACGAAAGGG
CCTCGTGATACGCCTATTTTTATAGGTTAATGTCATGATAATAATGGTTTCTTAGACGTCAG
GTGGCACTTTTCGGGGAAATGTGCGCGGAACCCCTATTTGTTTATTTTTCTAAATACATTCA
AATATGTATCCGCTCATGAGACAATAACCCTGATAAATGCTTCAATAATATTGAAAAAGGAA
GAGTATGAGTATTCAACATTTCCGTGTCGCCCTTATTCCCTTTTTTGCGGCATTTTGCCTTC
CTGTTTTTGCTCACCCAGAAACGCTGGTGAAAGTAAAAGATGCTGAAGATCAGTTGGGTGCA
CGAGTGGGTTACATCGAACTGGATCTCAACAGCGGTAAGATCCTTGAGAGTTTTCGCCCCGA
AGAACGTTTTCCAATGATGAGCACTTTTAAAGTTCTGCTATGTGGCGCGGTATTATCCCGTA
TTGACGCCGGGCAAGAGCAACTCGGTCGCCGCATACACTATTCTCAGAATGACTTGGTTGAG
TACTCACCAGTCACAGAAAAGCATCTTACGGATGGCATGACAGTAAGAGAATTATGCAGTGC
TGCCATAACCATGAGTGATAACACTGCGGCCAACTTACTTCTGACAACGATCGGAGGACCGA
AGGAGCTAACCGCTTTTTTGCACAACATGGGGGATCATGTAACTCGCCTTGATCGTTGGGAA
CCGGAGCTGAATGAAGCCATACCAAACGACGAGCGTGACACCA

In some embodiments, an AAV vector described herein comprises an AAV 5′ ITR, a GJB2 GRE enhancer (hGJB2 GRE4), a GJB2 basal promoter, a GJB2 exon 1 5′ UTR, Kozak sequence, nucleotide sequence encoding a gene product (e.g., GJB2 or GFP), a GJB2 exon 2 3′ UTR, a WPRE, a bovine growth hormone poly A signal, and an AAV 3′ ITR (e.g., vector c.81.4).

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 72. An exemplary nucleotide sequence for vector c.81.4 is set forth in SEQ ID NO: 72 (hGJB2 GRE4 underlined; eGFP coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTA
ACTGGGCAATGCGTTAAACTGGCTTTTTTGACTTCCCAGAACAATATCTAATTAGCAAATAA
CACAATTCAGTGACATTCAGCAGGATGCAAATTCCAGACACTGCAATCATGAACACTGTGAA
GACAGTCTTCTCCGTGGGCCGGGACACAAAGCAGTCCACAGTGTTGGGACAAGGCCAGGCGT
TGCACTTCACCAGCCGCTGCATGGAGAAGCCGTCGTACATGACATAGAAGACGTACATGAAG
GCGGCTTCGAAGATGACCCGGAAGAAGATGCTGCTTGTGTAGGTCCACCACAGGGAGCCTTC
GATGCGGACCTTCTGGGTTTTGATCTCCTCGATGTCCTTAAATTCACTCTTTATCTCCCCCT
TGATGAACTTCCTCTTCTTCTCATGTCTCCGGTAGGCCACGTGCATGGCCACTAGGAGCGCT
GGCGTGGACACGAAGATCAGCTGCAGGGCCCATAGCCGGATGTGGGAGATGGGGAAGTAGTG
ATCGTAGCACACGTTCTTGCAGCCTGGCTGCAGGGTGTTGCAGACAAAGTCGGCCTGCTCAT
CTCCCCACACCTCCTTTGCAGCCACAACGAGGATCATAATGCGAAAAATGAAGAGGACGGTG
AGCCAGATCTTTCCAATGCTGGTGGAGTGTTTGTTCACACCCCCCAGGATCGTCTGCAGCGT
GCCCCAATCCATCTTCTACTCTGGGCGGTTTGCTCTGGAAAAGACGAATGCACACAACACAG
GAATCACTAGCTAGGACAGAACAGGGAGACTTCTCTGAGTCTGGGTAAGCAAGCATGCTTAA
ATCTCTTCCTGAGCAAACACCAACTCTTACACAACCTCACCAAAACAGGTGAAGACAGAACC
AACTTAGTTTGTCATTAATTAAGACCTCGAAGGGGACTTGGGGGGTTCGGGGCTTTCGGGGG
CGGTCGGGGGTTCGCGGACCCGGGAAGCTCTGAGGACCCAGAGGCCGGGCGCGCTCCGCCCG
CGGCGCCGCCCCCTCCGTAACTTTCCCAGTCTCCGAGGGAAGAGGCGGGGTGTGGGGTGCGG
TTAAAAGGCGCCACGGCGGGAGACAGGTGTTGCGGCCCCGCAGCGCCCGCGCGCTCCTCTCC
CCGACTCGGAGCCCCTCGGCGGCGCCCGGCCCAGGACCCGCCTAGGAGCGCAGGAGCCCCAG
CGCAGAGACCCCAACGCCGAGACCCCCGCCCCGGCCCCGCCGCGCTTCCTCCCGACGCAGAG
CAAACCGCCCAGAGTAGAAGCCATGGTGAGCAAGGGCGAGGAGCTGTTCACCGGGGTGGTGC
CCATCCTGGTCGAGCTGGACGGCGACGTAAACGGCCACAAGTTCAGCGTGTCCGGCGAGGGC
GAGGGCGATGCCACCTACGGCAAGCTGACCCTGAAGTTCATCTGCACCACCGGCAAGCTGCC
CGTGCCCTGGCCCACCCTCGTGACCACCCTGACCTACGGCGTGCAGTGCTTCAGCCGCTACC
CCGACCACATGAAGCAGCACGACTTCTTCAAGTCCGCCATGCCCGAAGGCTACGTCCAGGAG
CGCACCATCTTCTTCAAGGACGACGGCAACTACAAGACCCGCGCCGAGGTGAAGTTCGAGGG
CGACACCCTGGTGAACCGCATCGAGCTGAAGGGCATCGACTTCAAGGAGGACGGCAACATCC
TGGGGCACAAGCTGGAGTACAACTACAACAGCCACAACGTCTATATCATGGCCGACAAGCAG
AAGAACGGCATCAAGGTGAACTTCAAGATCCGCCACAACATCGAGGACGGCAGCGTGCAGCT
CGCCGACCACTACCAGCAGAACACCCCCATCGGCGACGGCCCCGTGCTGCTGCCCGACAACC
ACTACCTGAGCACCCAGTCCGCCCTGAGCAAAGACCCCAACGAGAAGCGCGATCACATGGTC
CTGCTGGAGTTCGTGACCGCCGCCGGGATCACTCTCGGCATGGACGAGCTGTACAAGTAATA
AAGGCGCGCCACCCCTGCAGGGAATTCCGCATTGCCCAGTTGTTAGATTAAGAAATAGACAG
CATGAGAGGGATGAGGCAACCCGTGCTCAGCTGTCAAGGCTCAGTCGCTAGCATTTCCCAAC
ACAAAGATTCTGACCTTAAATGCAACCATTTGAAACCCCTGTAGGCCTCAGGTGAAACTCCA
GATGCCACAATGGAGCTCTGCTCCCCTAAAGCCTCAAAACAAAGGCCTAATTCTATGCCTGT
CTTAATTTTCTTTCACTTAAGTTAGTTCCACTGAGACCCCAGGCTGTTAGGGGTTATTGGTG
TAAGGTACTTTCATATTTTAAACAGAGGATATCGGCATTTGTTTCTTTCTCTGAGGACAAGA
GAAAAAAGCCAGGTTCCACAGAGGACACAGAGAAGGTTTGGGTGTCCTCCTGGGGTTCTTTT
TGCCAACTTTCCCCACGTTAAAGGTGAACATTGGTTCTTTCATTTGCTTTGGAAGTTTTAAT
CTCTAACAGTGGACAAAGTTACCAGTGCCTTAAACTCTGTTACACTTTTTGGAAGTGAAAAC
TTTGTAGTATGATAGGTTATTTTGATGTAAAGATGTTCTGGATACCATTATATGTTCCCCCT
GTTTCAGAGGCTCAGATTGTAATATGTAAATGGTATGTCATTCGCTACTATGATTTAATTTG
AAATATGGTCTTTTGGTTATGAATACTTTGCAGCACAGCTGAGAGGCTGTCTGTTGTATTCA
TTGTGGTCATAGCACCTAACAACATTGTAGCCTCAATCGAGTGAGACAGACTAGAAGTTCCT
AGTGATGGCTTATGATAGCAAATGGCCTCATGTCAAATATTTAGATGTAATTTTGTGTAAGA
AATACAGACTGGATGTACCACCAACTACTACCTGTAATGACAGGCCTGTCCAACACATCTCC
CTTTTCCATGACTGTGGTAGCCAGCATCGGAAAGAACGCTGATTTAAAGAGGTCGCTTGGGA
ATTTTATTGACACAGTACCATTTAATGGGGAGGACAAAATGGGGCAGGGGAGGGAGAAGTTT
CTGTCGTTAAAAACAGATTTGGAAAGACTGGACTCTAAAGTCTGTTGATTAAAGATGAGCTT
TGTCTACTTCAAAAGTTTGTTTGCTTACCCCTTCAGCCTCCAATTTTTTAAGTGAAAATATA
GCTAATAACATGTGAAAAGAATAGAAGCTAAGGTTTAGATAAATATTGAGCAGATCTATAGG
AAGATTGAACCTGAATATTGCCATTATGCTTGACATGGTTTCCAAAAAATGGTACTCCACAT
ATTTCAGTGAGGGTAAGTATTTTCCTGTTGTCAAGAATAGCATTGTAAAAGCATTTTGTAAT
AATAAAGAATAGCTTTAATGATATGCTTGTAACTAAAATAATTTTGTAATGTATCAAATACA
TTTAAAACATTAAAATATAATCTCTATAATAATTTAAAATCTAATATGGTTTTAATAGAACA
GCGATATCAAGCTTATCGATAATCAACCTCTGGATTACAAAATTTGTGAAAGATTGACTGGT
ATTCTTAACTATGTTGCTCCTTTTACGCTATGTGGATACGCTGCTTTAATGCCTTTGTATCA
TGCTATTGCTTCCCGTATGGCTTTCATTTTCTCCTCCTTGTATAAATCCTGGTTGCTGTCTC
TTTATGAGGAGTTGTGGCCCGTTGTCAGGCAACGTGGCGTGGTGTGCACTGTGTTTGCTGAC
GCAACCCCCACTGGTTGGGGCATTGCCACCACCTGTCAGCTCCTTTCCGGGACTTTCGCTTT
CCCCCTCCCTATTGCCACGGCGGAACTCATCGCCGCCTGCCTTGCCCGCTGCTGGACAGGGG
CTCGGCTGTTGGGCACTGACAATTCCGTGGTGTTGTCGGGGAAATCATCGTCCTTTCCTTGG
CTGCTCGCCTATGTTGCCACCTGGATTCTGCGCGGGACGTCCTTCTGCTACGTCCCTTCGGC
CCTCAATCCAGCGGACCTTCCTTCCCGCGGCCTGCTGCCGGCTCTGCGGCCTCTTCCGCGTC
TTCGCCTTCGCCCTCAGACGAGTCGGATCTCCCTTTGGGCCGCCTCCCCGCGAATTCATCGA
TACCGAGCGCTGCTCGAGAGATCTGTGATAGCGGCCATCAAGCTGGCTGTGCCTTCTAGTTG
CCAGCCATCTGTTGTTTGCCCCTCCCCCGTGCCTTCCTTGACCCTGGAAGGTGCCACTCCCA
CTGTCCTTTCCTAATAAAATGAGGAAATTGCATCGCATTGTCTGAGTAGGTGTCATTCTATT
CTGGGGGGTGGGGTGGGGCAGGACAGCAAGGGGGAGGATTGGGAAGACAATAGCAGGCATGC
TGGGGACACGTGCGGACCGAGCGGCCGCAGGAACCCCTAGTGATGGAGTTGGCCACTCCCTC
TCTGCGCGCTCGCTCGCTCACTGAGGCCGGGCGACCAAAGGTCGCCCGACGCCCGGGCTTTG
CCCGGGCGGCCTCAGTGAGCGAGCGAGCGCGCAGCTGCCTGCAGGGGCGCCTGATGCGGTAT
TTTCTCCTTACGCATCTGTGCGGTATTTCACACCGCATACGTCAAAGCAACCATAGTACGCG
CCCTGTAGCGGCGCATTAAGCGCGGCGGGTGTGGTGGTTACGCGCAGCGTGACCGCTACACT
TGCCAGCGCCCTAGCGCCCGCTCCTTTCGCTTTCTTCCCTTCCTTTCTCGCCACGTTCGCCG
GCTTTCCCCGTCAAGCTCTAAATCGGGGGCTCCCTTTAGGGTTCCGATTTAGTGCTTTACGG
CACCTCGACCCCAAAAAACTTGATTTGGGTGATGGTTCACTTAGTGGGCCATCGCCCTGATA
GACGGTTTTTCGCCCTTTGACGTTGGAGTCCACGTTCTTTAATAGTGGACTCTTGTTCCAAA
CTGGAACAACACTCAACCCTATCTCGGGCTATTCTTTTGATTTATAAGGGATTTTGCCGATT
TCGGCCTATTGGTTAAAAAATGAGCTGATTTAACAAAAATTTAACGCGAATTTTAACAAAAT
ATTAACGTTTACAATTTTATGGTGCACTCTCAGTACAATCTGCTCTGATGCCGCATAGTTAA
GCCAGCCCCGACACCCGCCAACACCCGCTGACGCGCCCTGACGGGCTTGTCTGCTCCCGGCA
TCCGCTTACAGACAAGCTGTGACCGTCTCCGGGAGCTGCATGTGTCAGAGGTTTTCACCGTC
ATCACCGAAACGCGCGAGACGAAAGGGCCTCGTGATACGCCTATTTTTATAGGTTAATGTCA
TGATAATAATGGTTTCTTAGACGTCAGGTGGCACTTTTCGGGGAAATGTGCGCGGAACCCCT
ATTTGTTTATTTTTCTAAATACATTCAAATATGTATCCGCTCATGAGACAATAACCCTGATA
AATGCTTCAATAATATTGAAAAAGGAAGAGTATGAGTATTCAACATTTCCGTGTCGCCCTTA
TTCCCTTTTTTGCGGCATTTTGCCTTCCTGTTTTTGCTCACCCAGAAACGCTGGTGAAAGTA
AAAGATGCTGAAGATCAGTTGGGTGCACGAGTGGGTTACATCGAACTGGATCTCAACAGCGG
TAAGATCCTTGAGAGTTTTCGCCCCGAAGAACGTTTTCCAATGATGAGCACTTTTAAAGTTC
TGCTATGTGGCGCGGTATTATCCCGTATTGACGCCGGGCAAGAGCAACTCGGTCGCCGCATA
CACTATTCTCAGAATGACTTGGTTGAGTACTCACCAGTCACAGAAAAGCATCTTACGGATGG
CATGACAGTAAGAGAATTATGCAGTGCTGCCATAACCATGAGTGATAACACTGCGGCCAACT
TACTTCTGACAACGATCGGAGGACCGAAGGAGCTAACCGCTTTTTTGCACAACATGGGGGAT
CATGTAACTCGCCTTGATCGTTGGGAACCGGAGCTGAATGAAGCCATACCAAACGACGAGCG
TGACACCA

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 73. An exemplary nucleotide sequence for vector c.81.4 is set forth in SEQ ID NO: 73 (hGJB2 GRE4 underlined; human GJB2 coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTA
ACTGGGCAATGCGTTAAACTGGCTTTTTTGACTTCCCAGAACAATATCTAATTAGCAAATAA
CACAATTCAGTGACATTCAGCAGGATGCAAATTCCAGACACTGCAATCATGAACACTGTGAA
GACAGTCTTCTCCGTGGGCCGGGACACAAAGCAGTCCACAGTGTTGGGACAAGGCCAGGCGT
TGCACTTCACCAGCCGCTGCATGGAGAAGCCGTCGTACATGACATAGAAGACGTACATGAAG
GCGGCTTCGAAGATGACCCGGAAGAAGATGCTGCTTGTGTAGGTCCACCACAGGGAGCCTTC
GATGCGGACCTTCTGGGTTTTGATCTCCTCGATGTCCTTAAATTCACTCTTTATCTCCCCCT
TGATGAACTTCCTCTTCTTCTCATGTCTCCGGTAGGCCACGTGCATGGCCACTAGGAGCGCT
GGCGTGGACACGAAGATCAGCTGCAGGGCCCATAGCCGGATGTGGGAGATGGGGAAGTAGTG
ATCGTAGCACACGTTCTTGCAGCCTGGCTGCAGGGTGTTGCAGACAAAGTCGGCCTGCTCAT
CTCCCCACACCTCCTTTGCAGCCACAACGAGGATCATAATGCGAAAAATGAAGAGGACGGTG
AGCCAGATCTTTCCAATGCTGGTGGAGTGTTTGTTCACACCCCCCAGGATCGTCTGCAGCGT
GCCCCAATCCATCTTCTACTCTGGGCGGTTTGCTCTGGAAAAGACGAATGCACACAACACAG
GAATCACTAGCTAGGACAGAACAGGGAGACTTCTCTGAGTCTGGGTAAGCAAGCATGCTTAA
ATCTCTTCCTGAGCAAACACCAACTCTTACACAACCTCACCAAAACAGGTGAAGACAGAACC
AACTTAGTTTGTCATTAATTAAGACCTCGAAGGGGACTTGGGGGGTTCGGGGCTTTCGGGGG
CGGTCGGGGGTTCGCGGACCCGGGAAGCTCTGAGGACCCAGAGGCCGGGCGCGCTCCGCCCG
CGGCGCCGCCCCCTCCGTAACTTTCCCAGTCTCCGAGGGAAGAGGCGGGGTGTGGGGTGCGG
TTAAAAGGCGCCACGGCGGGAGACAGGTGTTGCGGCCCCGCAGCGCCCGCGCGCTCCTCTCC
CCGACTCGGAGCCCCTCGGCGGCGCCCGGCCCAGGACCCGCCTAGGAGCGCAGGAGCCCCAG
CGCAGAGACCCCAACGCCGAGACCCCCGCCCCGGCCCCGCCGCGCTTCCTCCCGACGCAGAG
CAAACCGCCCAGAGTAGAAGCCATGGATTGGGGCACGCTGCAGACGATCCTGGGGGGTGTGA
ACAAACACTCCACCAGCATTGGAAAGATCTGGCTCACCGTCCTCTTCATTTTTCGCATTATG
ATCCTCGTTGTGGCTGCAAAGGAGGTGTGGGGAGATGAGCAGGCCGACTTTGTCTGCAACAC
CCTGCAGCCAGGCTGCAAGAACGTGTGCTACGATCACTACTTCCCCATCTCCCACATCCGGC
TATGGGCCCTGCAGCTGATCTTCGTGTCCACGCCAGCGCTCCTAGTGGCCATGCACGTGGCC
TACCGGAGACATGAGAAGAAGAGGAAGTTCATCAAGGGGGAGATAAAGAGTGAATTTAAGGA
CATCGAGGAGATCAAAACCCAGAAGGTCCGCATCGAAGGCTCCCTGTGGTGGACCTACACAA
GCAGCATCTTCTTCCGGGTCATCTTCGAAGCCGCCTTCATGTACGTCTTCTATGTCATGTAC
GACGGCTTCTCCATGCAGCGGCTGGTGAAGTGCAACGCCTGGCCTTGTCCCAACACTGTGGA
CTGCTTTGTGTCCCGGCCCACGGAGAAGACTGTCTTCACAGTGTTCATGATTGCAGTGTCTG
GAATTTGCATCCTGCTGAATGTCACTGAATTGTGTTATTTGCTAATTAGATATTGTTCTGGG
AAGTCAAAAAAGCCAGTTTAAAGGCGCGCCACCCCTGCAGGGAATTCCGCATTGCCCAGTTG
TTAGATTAAGAAATAGACAGCATGAGAGGGATGAGGCAACCCGTGCTCAGCTGTCAAGGCTC
AGTCGCTAGCATTTCCCAACACAAAGATTCTGACCTTAAATGCAACCATTTGAAACCCCTGT
AGGCCTCAGGTGAAACTCCAGATGCCACAATGGAGCTCTGCTCCCCTAAAGCCTCAAAACAA
AGGCCTAATTCTATGCCTGTCTTAATTTTCTTTCACTTAAGTTAGTTCCACTGAGACCCCAG
GCTGTTAGGGGTTATTGGTGTAAGGTACTTTCATATTTTAAACAGAGGATATCGGCATTTGT
TTCTTTCTCTGAGGACAAGAGAAAAAAGCCAGGTTCCACAGAGGACACAGAGAAGGTTTGGG
TGTCCTCCTGGGGTTCTTTTTGCCAACTTTCCCCACGTTAAAGGTGAACATTGGTTCTTTCA
TTTGCTTTGGAAGTTTTAATCTCTAACAGTGGACAAAGTTACCAGTGCCTTAAACTCTGTTA
CACTTTTTGGAAGTGAAAACTTTGTAGTATGATAGGTTATTTTGATGTAAAGATGTTCTGGA
TACCATTATATGTTCCCCCTGTTTCAGAGGCTCAGATTGTAATATGTAAATGGTATGTCATT
CGCTACTATGATTTAATTTGAAATATGGTCTTTTGGTTATGAATACTTTGCAGCACAGCTGA
GAGGCTGTCTGTTGTATTCATTGTGGTCATAGCACCTAACAACATTGTAGCCTCAATCGAGT
GAGACAGACTAGAAGTTCCTAGTGATGGCTTATGATAGCAAATGGCCTCATGTCAAATATTT
AGATGTAATTTTGTGTAAGAAATACAGACTGGATGTACCACCAACTACTACCTGTAATGACA
GGCCTGTCCAACACATCTCCCTTTTCCATGACTGTGGTAGCCAGCATCGGAAAGAACGCTGA
TTTAAAGAGGTCGCTTGGGAATTTTATTGACACAGTACCATTTAATGGGGAGGACAAAATGG
GGCAGGGGAGGGAGAAGTTTCTGTCGTTAAAAACAGATTTGGAAAGACTGGACTCTAAAGTC
TGTTGATTAAAGATGAGCTTTGTCTACTTCAAAAGTTTGTTTGCTTACCCCTTCAGCCTCCA
ATTTTTTAAGTGAAAATATAGCTAATAACATGTGAAAAGAATAGAAGCTAAGGTTTAGATAA
ATATTGAGCAGATCTATAGGAAGATTGAACCTGAATATTGCCATTATGCTTGACATGGTTTC
CAAAAAATGGTACTCCACATATTTCAGTGAGGGTAAGTATTTTCCTGTTGTCAAGAATAGCA
TTGTAAAAGCATTTTGTAATAATAAAGAATAGCTTTAATGATATGCTTGTAACTAAAATAAT
TTTGTAATGTATCAAATACATTTAAAACATTAAAATATAATCTCTATAATAATTTAAAATCT
AATATGGTTTTAATAGAACAGCGATATCAAGCTTATCGATAATCAACCTCTGGATTACAAAA
TTTGTGAAAGATTGACTGGTATTCTTAACTATGTTGCTCCTTTTACGCTATGTGGATACGCT
GCTTTAATGCCTTTGTATCATGCTATTGCTTCCCGTATGGCTTTCATTTTCTCCTCCTTGTA
TAAATCCTGGTTGCTGTCTCTTTATGAGGAGTTGTGGCCCGTTGTCAGGCAACGTGGCGTGG
TGTGCACTGTGTTTGCTGACGCAACCCCCACTGGTTGGGGCATTGCCACCACCTGTCAGCTC
CTTTCCGGGACTTTCGCTTTCCCCCTCCCTATTGCCACGGCGGAACTCATCGCCGCCTGCCT
TGCCCGCTGCTGGACAGGGGCTCGGCTGTTGGGCACTGACAATTCCGTGGTGTTGTCGGGGA
AATCATCGTCCTTTCCTTGGCTGCTCGCCTATGTTGCCACCTGGATTCTGCGCGGGACGTCC
TTCTGCTACGTCCCTTCGGCCCTCAATCCAGCGGACCTTCCTTCCCGCGGCCTGCTGCCGGC
TCTGCGGCCTCTTCCGCGTCTTCGCCTTCGCCCTCAGACGAGTCGGATCTCCCTTTGGGCCG
CCTCCCCGCGAATTCATCGATACCGAGCGCTGCTCGAGAGATCTGTGATAGCGGCCATCAAG
CTGGCTGTGCCTTCTAGTTGCCAGCCATCTGTTGTTTGCCCCTCCCCCGTGCCTTCCTTGAC
CCTGGAAGGTGCCACTCCCACTGTCCTTTCCTAATAAAATGAGGAAATTGCATCGCATTGTC
TGAGTAGGTGTCATTCTATTCTGGGGGGTGGGGTGGGGCAGGACAGCAAGGGGGAGGATTGG
GAAGACAATAGCAGGCATGCTGGGGACACGTGCGGACCGAGCGGCCGCAGGAACCCCTAGTG
ATGGAGTTGGCCACTCCCTCTCTGCGCGCTCGCTCGCTCACTGAGGCCGGGCGACCAAAGGT
CGCCCGACGCCCGGGCTTTGCCCGGGCGGCCTCAGTGAGCGAGCGAGCGCGCAGCTGCCTGC
AGGGGCGCCTGATGCGGTATTTTCTCCTTACGCATCTGTGCGGTATTTCACACCGCATACGT
CAAAGCAACCATAGTACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTTCCCTTC
CTTTCTCGCCACGTTCGCCGGCTTTCCCCGTCAAGCTCTAAATCGGGGGCTCCCTTTAGGGT
TCCGATTTAGTGCTTTACGGCACCTCGACCCCAAAAAACTTGATTTGGGTGATGGTTCACGT
AGTGGGCCATCGCCCTGATAGACGGTTTTTCGCCCTTTGACGTTGGAGTCCACGTTCTTTAA
TAGTGGACTCTTGTTCCAAACTGGAACAACACTCAACCCTATCTCGGGCTATTCTTTTGATT
TATAAGGGATTTTGCCGATTTCGGCCTATTGGTTAAAAAATGAGCTGATTTAACAAAAATTT
AACGCGAATTTTAACAAAATATTAACGTTTACAATTTTATGGTGCACTCTCAGTACAATCTG
CTCTGATGCCGCATAGTTAAGCCAGCCCCGACACCCGCCAACACCCGCTGACGCGCCCTGAC
GGGCTTGTCTGCTCCCGGCATCCGCTTACAGACAAGCTGTGACCGTCTCCGGGAGCTGCATG
TGTCAGAGGTTTTCACCGTCATCACCGAAACGCGCGAGACGAAAGGGCCTCGTGATACGCCT
ATTTTTATAGGTTAATGTCATGATAATAATGGTTTCTTAGACGTCAGGTGGCACTTTTCGGG
GAAATGTGCGCGGAACCCCTATTTGTTTATTTTTCTAAATACATTCAAATATGTATCCGCTC
ATGAGACAATAACCCTGATAAATGCTTCAATAATATTGAAAAAGGAAGAGTATGAGTATTCA
ACATTTCCGTGTCGCCCTTATTCCCTTTTTTGCGGCATTTTGCCTTCCTGTTTTTGCTCACC
CAGAAACGCTGGTGAAAGTAAAAGATGCTGAAGATCAGTTGGGTGCACGAGTGGGTTACATC
GAACTGGATCTCAACAGCGGTAAGATCCTTGAGAGTTTTCGCCCCGAAGAACGTTTTCCAAT
GATGAGCACTTTTAAAGTTCTGCTATGTGGCGCGGTATTATCCCGTATTGACGCCGGGCAAG
AGCAACTCGGTCGCCGCATACACTATTCTCAGAATGACTTGGTTGAGTACTCACCAGTCACA
GAAAAGCATCTTACGGATGGCATGACAGTAAGAGAATTATGCAGTGCTGCCATAACCATGAG
TGATAACACTGCGGCCAACTTACTTCTGACAACGATCGGAGGACCGAAGGAGCTAACCGCTT
TTTTGCACAACATGGGGGATCATGTAACTCGCCTTGATCGTTGGGAACCGGAGCTGAATGAA
GCCATACCAAACGACGAGCGTGACACCA

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 74. An exemplary nucleotide sequence for vector c.81.4 is set forth in SEQ ID NO: 74 (hGJB2 GRE4 underlined; mouse GJB2 coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTA
ACTGGGCAATGCGTTAAACTGGCTTTTTTGACTTCCCAGAACAATATCTAATTAGCAAATAA
CACAATTCAGTGACATTCAGCAGGATGCAAATTCCAGACACTGCAATCATGAACACTGTGAA
GACAGTCTTCTCCGTGGGCCGGGACACAAAGCAGTCCACAGTGTTGGGACAAGGCCAGGCGT
TGCACTTCACCAGCCGCTGCATGGAGAAGCCGTCGTACATGACATAGAAGACGTACATGAAG
GCGGCTTCGAAGATGACCCGGAAGAAGATGCTGCTTGTGTAGGTCCACCACAGGGAGCCTTC
GATGCGGACCTTCTGGGTTTTGATCTCCTCGATGTCCTTAAATTCACTCTTTATCTCCCCCT
TGATGAACTTCCTCTTCTTCTCATGTCTCCGGTAGGCCACGTGCATGGCCACTAGGAGCGCT
GGCGTGGACACGAAGATCAGCTGCAGGGCCCATAGCCGGATGTGGGAGATGGGGAAGTAGTG
ATCGTAGCACACGTTCTTGCAGCCTGGCTGCAGGGTGTTGCAGACAAAGTCGGCCTGCTCAT
CTCCCCACACCTCCTTTGCAGCCACAACGAGGATCATAATGCGAAAAATGAAGAGGACGGTG
AGCCAGATCTTTCCAATGCTGGTGGAGTGTTTGTTCACACCCCCCAGGATCGTCTGCAGCGT
GCCCCAATCCATCTTCTACTCTGGGCGGTTTGCTCTGGAAAAGACGAATGCACACAACACAG
GAATCACTAGCTAGGACAGAACAGGGAGACTTCTCTGAGTCTGGGTAAGCAAGCATGCTTAA
ATCTCTTCCTGAGCAAACACCAACTCTTACACAACCTCACCAAAACAGGTGAAGACAGAACC
AACTTAGTTTGTCATTAATTAAGACCTCGAAGGGGACTTGGGGGGTTCGGGGCTTTCGGGGG
CGGTCGGGGGTTCGCGGACCCGGGAAGCTCTGAGGACCCAGAGGCCGGGCGCGCTCCGCCCG
CGGCGCCGCCCCCTCCGTAACTTTCCCAGTCTCCGAGGGAAGAGGCGGGGTGTGGGGTGCGG
TTAAAAGGCGCCACGGCGGGAGACAGGTGTTGCGGCCCCGCAGCGCCCGCGCGCTCCTCTCC
CCGACTCGGAGCCCCTCGGCGGCGCCCGGCCCAGGACCCGCCTAGGAGCGCAGGAGCCCCAG
CGCAGAGACCCCAACGCCGAGACCCCCGCCCCGGCCCCGCCGCGCTTCCTCCCGACGCAGAG
CAAACCGCCCAGAGTAGAAGCCATGGATTGGGGCACACTCCAGAGCATCCTCGGGGGTGTCA
ACAAACACTCCACCAGCATTGGAAAGATCTGGCTCACGGTCCTCTTCATCTTCCGCATCATG
ATCCTCGTGGTGGCTGCAAAGGAGGTGTGGGGAGATGAGCAAGCCGATTTTGTCTGCAACAC
GCTCCAGCCTGGCTGCAAGAATGTATGCTACGACCACCACTTCCCCATCTCTCACATCCGGC
TCTGGGCTCTGCAGCTGATCATGGTGTCCACGCCAGCCCTCCTGGTAGCTATGCATGTGGCC
TACCGGAGACATGAAAAGAAACGGAAGTTCATGAAGGGAGAGATAAAGAACGAGTTTAAGGA
CATCGAAGAGATCAAAACCCAGAAGGTCCGTATCGAAGGGTCCCTGTGGTGGACCTACACCA
CCAGCATCTTCTTCCGGGTCATCTTTGAAGCCGTCTTCATGTACGTCTTTTACATCATGTAC
AATGGCTTCTTCATGCAACGTCTGGTGAAATGCAACGCTTGGCCCTGCCCCAATACAGTGGA
CTGCTTCATTTCCAGGCCCACAGAAAAGACTGTCTTCACCGTGTTTATGATTTCTGTGTCTG
GAATTTGCATTCTGCTAAATATCACAGAGCTGTGCTATTTGTTCGTTAGGTATTGCTCAGGA
AAGTCCAAAAGACCAGTCTACCCATACGATGTTCCAGATTACGCTTAAAGGCGCGCCACCCC
TGCAGGGAATTCCGCATTGCCCAGTTGTTAGATTAAGAAATAGACAGCATGAGAGGGATGAG
GCAACCCGTGCTCAGCTGTCAAGGCTCAGTCGCTAGCATTTCCCAACACAAAGATTCTGACC
TTAAATGCAACCATTTGAAACCCCTGTAGGCCTCAGGTGAAACTCCAGATGCCACAATGGAG
CTCTGCTCCCCTAAAGCCTCAAAACAAAGGCCTAATTCTATGCCTGTCTTAATTTTCTTTCA
CTTAAGTTAGTTCCACTGAGACCCCAGGCTGTTAGGGGTTATTGGTGTAAGGTACTTTCATA
TTTTAAACAGAGGATATCGGCATTTGTTTCTTTCTCTGAGGACAAGAGAAAAAAGCCAGGTT
CCACAGAGGACACAGAGAAGGTTTGGGTGTCCTCCTGGGGTTCTTTTTGCCAACTTTCCCCA
CGTTAAAGGTGAACATTGGTTCTTTCATTTGCTTTGGAAGTTTTAATCTCTAACAGTGGACA
AAGTTACCAGTGCCTTAAACTCTGTTACACTTTTTGGAAGTGAAAACTTTGTAGTATGATAG
GTTATTTTGATGTAAAGATGTTCTGGATACCATTATATGTTCCCCCTGTTTCAGAGGCTCAG
ATTGTAATATGTAAATGGTATGTCATTCGCTACTATGATTTAATTTGAAATATGGTCTTTTG
GTTATGAATACTTTGCAGCACAGCTGAGAGGCTGTCTGTTGTATTCATTGTGGTCATAGCAC
CTAACAACATTGTAGCCTCAATCGAGTGAGACAGACTAGAAGTTCCTAGTGATGGCTTATGA
TAGCAAATGGCCTCATGTCAAATATTTAGATGTAATTTTGTGTAAGAAATACAGACTGGATG
TACCACCAACTACTACCTGTAATGACAGGCCTGTCCAACACATCTCCCTTTTCCATGACTGT
GGTAGCCAGCATCGGAAAGAACGCTGATTTAAAGAGGTCGCTTGGGAATTTTATTGACACAG
TACCATTTAATGGGGAGGACAAAATGGGGCAGGGGAGGGAGAAGTTTCTGTCGTTAAAAACA
GATTTGGAAAGACTGGACTCTAAAGTCTGTTGATTAAAGATGAGCTTTGTCTACTTCAAAAG
TTTGTTTGCTTACCCCTTCAGCCTCCAATTTTTTAAGTGAAAATATAGCTAATAACATGTGA
AAAGAATAGAAGCTAAGGTTTAGATAAATATTGAGCAGATCTATAGGAAGATTGAACCTGAA
TATTGCCATTATGCTTGACATGGTTTCCAAAAAATGGTACTCCACATATTTCAGTGAGGGTA
AGTATTTTCCTGTTGTCAAGAATAGCATTGTAAAAGCATTTTGTAATAATAAAGAATAGCTT
TAATGATATGCTTGTAACTAAAATAATTTTGTAATGTATCAAATACATTTAAAACATTAAAA
TATAATCTCTATAATAATTTAAAATCTAATATGGTTTTAATAGAACAGCGATATCAAGCTTA
TCGATAATCAACCTCTGGATTACAAAATTTGTGAAAGATTGACTGGTATTCTTAACTATGTT
GCTCCTTTTACGCTATGTGGATACGCTGCTTTAATGCCTTTGTATCATGCTATTGCTTCCCG
TATGGCTTTCATTTTCTCCTCCTTGTATAAATCCTGGTTGCTGTCTCTTTATGAGGAGTTGT
GGCCCGTTGTCAGGCAACGTGGCGTGGTGTGCACTGTGTTTGCTGACGCAACCCCCACTGGT
TGGGGCATTGCCACCACCTGTCAGCTCCTTTCCGGGACTTTCGCTTTCCCCCTCCCTATTGC
CACGGCGGAACTCATCGCCGCCTGCCTTGCCCGCTGCTGGACAGGGGCTCGGCTGTTGGGCA
CTGACAATTCCGTGGTGTTGTCGGGGAAATCATCGTCCTTTCCTTGGCTGCTCGCCTATGTT
GCCACCTGGATTCTGCGCGGGACGTCCTTCTGCTACGTCCCTTCGGCCCTCAATCCAGCGGA
CCTTCCTTCCCGCGGCCTGCTGCCGGCTCTGCGGCCTCTTCCGCGTCTTCGCCTTCGCCCTC
AGACGAGTCGGATCTCCCTTTGGGCCGCCTCCCCGCGAATTCATCGATACCGAGCGCTGCTC
GAGAGATCTGTGATAGCGGCCATCAAGCTGGCTGTGCCTTCTAGTTGCCAGCCATCTGTTGT
TTGCCCCTCCCCCGTGCCTTCCTTGACCCTGGAAGGTGCCACTCCCACTGTCCTTTCCTAAT
AAAATGAGGAAATTGCATCGCATTGTCTGAGTAGGTGTCATTCTATTCTGGGGGGTGGGGTG
GGGCAGGACAGCAAGGGGGAGGATTGGGAAGACAATAGCAGGCATGCTGGGGACACGTGCGG
ACCGAGCGGCCGCAGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCCCGGGCGGCCTCAG
TGAGCGAGCGAGCGCGCAGCTGCCTGCAGGGGCGCCTGATGCGGTATTTTCTCCTTACGCAT
CTGTGCGGTATTTCACACCGCATACGTCAAAGCAACCATAGTACGCGCCCTGTAGCGGCGCA
TTAAGCGCGGCGGGTGTGGTGGTTACGCGCAGCGTGACCGCTACACTTGCCAGCGCCCTAGC
GCCCGCTCCTTTCGCTTTCTTCCCTTCCTTTCTCGCCACGTTCGCCGGCTTTCCCCGTCAAG
CTCTAAATCGGGGGCTCCCTTTAGGGTTCCGATTTAGTGCTTTACGGCACCTCGACCCCAAA
AAACTTGATTTGGGTGATGGTTCACGTAGTGGGCCATCGCCCTGATAGACGGTTTTTCGCCC
TTTGACGTTGGAGTCCACGTTCTTTAATAGTGGACTCTTGTTCCAAACTGGAACAACACTCA
ACCCTATCTCGGGCTATTCTTTTGATTTATAAGGGATTTTGCCGATTTCGGCCTATTGGTTA
AAAAATGAGCTGATTTAACAAAAATTTAACGCGAATTTTAACAAAATATTAACGTTTACAAT
TTTATGGTGCACTCTCAGTACAATCTGCTCTGATGCCGCATAGTTAAGCCAGCCCCGACACC
CGCCAACACCCGCTGACGCGCCCTGACGGGCTTGTCTGCTCCCGGCATCCGCTTACAGACAA
GCTGTGACCGTCTCCGGGAGCTGCATGTGTCAGAGGTTTTCACCGTCATCACCGAAACGCGC
GAGACGAAAGGGCCTCGTGATACGCCTATTTTTATAGGTTAATGTCATGATAATAATGGTTT
CTTAGACGTCAGGTGGCACTTTTCGGGGAAATGTGCGCGGAACCCCTATTTGTTTATTTTTC
TAAATACATTCAAATATGTATCCGCTCATGAGACAATAACCCTGATAAATGCTTCAATAATA
TTGAAAAAGGAAGAGTATGAGTATTCAACATTTCCGTGTCGCCCTTATTCCCTTTTTTGCGG
CATTTTGCCTTCCTGTTTTTGCTCACCCAGAAACGCTGGTGAAAGTAAAAGATGCTGAAGAT
CAGTTGGGTGCACGAGTGGGTTACATCGAACTGGATCTCAACAGCGGTAAGATCCTTGAGAG
TTTTCGCCCCGAAGAACGTTTTCCAATGATGAGCACTTTTAAAGTTCTGCTATGTGGCGCGG
TATTATCCCGTATTGACGCCGGGCAAGAGCAACTCGGTCGCCGCATACACTATTCTCAGAAT
GACTTGGTTGAGTACTCACCAGTCACAGAAAAGCATCTTACGGATGGCATGACAGTAAGAGA
ATTATGCAGTGCTGCCATAACCATGAGTGATAACACTGCGGCCAACTTACTTCTGACAACGA
TCGGAGGACCGAAGGAGCTAACCGCTTTTTTGCACAACATGGGGGATCATGTAACTCGCCTT
GATCGTTGGGAACCGGAGCTGAATGAAGCCATACCAAACGACGAGCGTGACACCA

In some embodiments, an AAV vector described herein comprises an AAV 5′ ITR, a GJB2 GRE enhancer (hGJB2 GRE5), a GJB2 basal promoter, a GJB2 exon 1 5′ UTR, Kozak sequence, nucleotide sequence encoding a gene product (e.g., GJB2 or GFP), a GJB2 exon 2 3′ UTR, a WPRE, a bovine growth hormone poly A signal, and an AAV 3′ ITR (e.g., vector c.81.5).

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 50. An exemplary nucleotide sequence for vector c.81.5 is set forth in SEQ ID NO: 50 (hGJB2 GRE5 underlined; eGFP coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTA
AGCTACTAACTACAACCACGAGATTATAGATGTTTGCTGATATTGTTCTCAGTTTGGTTATT
GTGTTGTTTATGAATGAAAGTAGTGTATGTTTGTGTGAATTTTTGTTTTTAATTTTTTATGA
GTGCCCTAACAAAGATTACAAATTGGGAATACAAACTCCAGAGCAATGGAGACAGTGACACT
TTTGTGGAGGGGTACATGTGGCTGTTCGGGTGGTTATTAACACAGGCTGCTGCCCCTGCCCT
GCAATGGGAATCCCCAGGGCATTGGAGGATTCAACCTCTTGCAGTTACCTCTTGTAAGACAG
CAGATGGCAGCAGAGAGAGGCTTTGCACATCCCTGCAGGTTCTAGTTTGCACAAAGGGCTTC
TGAGAGACCTATCAACCAATTATAACATCAAGTGGCAAAAAGAGTCCTTGATAAGTTATTTC
GCTTCTCAAAGAAACCGAAAACGCCAAACTAATCACTAGTCTTGTTTTTTTTTTTCCTGGCA
AAAGCCTGCTATCTTTCATGATTTAGCTTTCATGAAATTGTTCCTGAAGACCCCCAAAAGAA
ACAATTTCATGCCCCGAACTCTGTTCAGAGACTTTGCTGTGCCTGTCATGTCCAGCTTGCCA
TATCCTGTTTTGTAAAGTAGCCACCTTATATACACACCTGCTGTCTGCACTGTGACCTCCTT
TCAAAATCATCTTTGGTTCTTCAGAGGCCTGGAATAATGCTCTGCCCAGATGAAGATCTCCG
TAAATGTGTTTTTGAAATGGCTAATCAAATAATGGATACCCTTAGGTATTTTTGCAGAAACA
CTTGGCAGCCTTCCATAATATCCCTACTATGAAATGGAAACTTGTGAATGAGATGTGGCTTT
AATTAAGACCTCGAAGGGGACTTGGGGGGTTCGGGGCTTTCGGGGGCGGTCGGGGGTTCGCG
GACCCGGGAAGCTCTGAGGACCCAGAGGCCGGGCGCGCTCCGCCCGCGGCGCCGCCCCCTCC
GTAACTTTCCCAGTCTCCGAGGGAAGAGGCGGGGTGTGGGGTGCGGTTAAAAGGCGCCACGG
CGGGAGACAGGTGTTGCGGCCCCGCAGCGCCCGCGCGCTCCTCTCCCCGACTCGGAGCCCCT
CGGCGGCGCCCGGCCCAGGACCCGCCTAGGAGCGCAGGAGCCCCAGCGCAGAGACCCCAACG
CCGAGACCCCCGCCCCGGCCCCGCCGCGCTTCCTCCCGACGCAGAGCAAACCGCCCAGAGTA
GAAGCCATGGTGAGCAAGGGCGAGGAGCTGTTCACCGGGGTGGTGCCCATCCTGGTCGAGCT
GGACGGCGACGTAAACGGCCACAAGTTCAGCGTGTCCGGCGAGGGCGAGGGCGATGCCACCT
ACGGCAAGCTGACCCTGAAGTTCATCTGCACCACCGGCAAGCTGCCCGTGCCCTGGCCCACC
CTCGTGACCACCCTGACCTACGGCGTGCAGTGCTTCAGCCGCTACCCCGACCACATGAAGCA
GCACGACTTCTTCAAGTCCGCCATGCCCGAAGGCTACGTCCAGGAGCGCACCATCTTCTTCA
AGGACGACGGCAACTACAAGACCCGCGCCGAGGTGAAGTTCGAGGGCGACACCCTGGTGAAC
CGCATCGAGCTGAAGGGCATCGACTTCAAGGAGGACGGCAACATCCTGGGGCACAAGCTGGA
GTACAACTACAACAGCCACAACGTCTATATCATGGCCGACAAGCAGAAGAACGGCATCAAGG
TGAACTTCAAGATCCGCCACAACATCGAGGACGGCAGCGTGCAGCTCGCCGACCACTACCAG
CAGAACACCCCCATCGGCGACGGCCCCGTGCTGCTGCCCGACAACCACTACCTGAGCACCCA
GTCCGCCCTGAGCAAAGACCCCAACGAGAAGCGCGATCACATGGTCCTGCTGGAGTTCGTGA
CCGCCGCCGGGATCACTCTCGGCATGGACGAGCTGTACAAGTAAAGGCGCGCCACCCCTGCA
GGGAATTCCGCATTGCCCAGTTGTTAGATTAAGAAATAGACAGCATGAGAGGGATGAGGCAA
CCCGTGCTCAGCTGTCAAGGCTCAGTCGCTAGCATTTCCCAACACAAAGATTCTGACCTTAA
ATGCAACCATTTGAAACCCCTGTAGGCCTCAGGTGAAACTCCAGATGCCACAATGGAGCTCT
GCTCCCCTAAAGCCTCAAAACAAAGGCCTAATTCTATGCCTGTCTTAATTTTCTTTCACTTA
AGTTAGTTCCACTGAGACCCCAGGCTGTTAGGGGTTATTGGTGTAAGGTACTTTCATATTTT
AAACAGAGGATATCGGCATTTGTTTCTTTCTCTGAGGACAAGAGAAAAAAGCCAGGTTCCAC
AGAGGACACAGAGAAGGTTTGGGTGTCCTCCTGGGGTTCTTTTTGCCAACTTTCCCCACGTT
AAAGGTGAACATTGGTTCTTTCATTTGCTTTGGAAGTTTTAATCTCTAACAGTGGACAAAGT
TACCAGTGCCTTAAACTCTGTTACACTTTTTGGAAGTGAAAACTTTGTAGTATGATAGGTTA
TTTTGATGTAAAGATGTTCTGGATACCATTATATGTTCCCCCTGTTTCAGAGGCTCAGATTG
TAATATGTAAATGGTATGTCATTCGCTACTATGATTTAATTTGAAATATGGTCTTTTGGTTA
TGAATACTTTGCAGCACAGCTGAGAGGCTGTCTGTTGTATTCATTGTGGTCATAGCACCTAA
CAACATTGTAGCCTCAATCGAGTGAGACAGACTAGAAGTTCCTAGTGATGGCTTATGATAGC
AAATGGCCTCATGTCAAATATTTAGATGTAATTTTGTGTAAGAAATACAGACTGGATGTACC
ACCAACTACTACCTGTAATGACAGGCCTGTCCAACACATCTCCCTTTTCCATGACTGTGGTA
GCCAGCATCGGAAAGAACGCTGATTTAAAGAGGTCGCTTGGGAATTTTATTGACACAGTACC
ATTTAATGGGGAGGACAAAATGGGGCAGGGGAGGGAGAAGTTTCTGTCGTTAAAAACAGATT
TGGAAAGACTGGACTCTAAAGTCTGTTGATTAAAGATGAGCTTTGTCTACTTCAAAAGTTTG
TTTGCTTACCCCTTCAGCCTCCAATTTTTTAAGTGAAAATATAGCTAATAACATGTGAAAAG
AATAGAAGCTAAGGTTTAGATAAATATTGAGCAGATCTATAGGAAGATTGAACCTGAATATT
GCCATTATGCTTGACATGGTTTCCAAAAAATGGTACTCCACATATTTCAGTGAGGGTAAGTA
TTTTCCTGTTGTCAAGAATAGCATTGTAAAAGCATTTTGTAATAATAAAGAATAGCTTTAAT
GATATGCTTGTAACTAAAATAATTTTGTAATGTATCAAATACATTTAAAACATTAAAATATA
ATCTCTATAATAATTTAAAATCTAATATGGTTTTAATAGAACAGCGATATCAAGCTTATCGA
TAATCAACCTCTGGATTACAAAATTTGTGAAAGATTGACTGGTATTCTTAACTATGTTGCTC
CTTTTACGCTATGTGGATACGCTGCTTTAATGCCTTTGTATCATGCTATTGCTTCCCGTATG
GCTTTCATTTTCTCCTCCTTGTATAAATCCTGGTTGCTGTCTCTTTATGAGGAGTTGTGGCC
CGTTGTCAGGCAACGTGGCGTGGTGTGCACTGTGTTTGCTGACGCAACCCCCACTGGTTGGG
GCATTGCCACCACCTGTCAGCTCCTTTCCGGGACTTTCGCTTTCCCCCTCCCTATTGCCACG
GCGGAACTCATCGCCGCCTGCCTTGCCCGCTGCTGGACAGGGGCTCGGCTGTTGGGCACTGA
CAATTCCGTGGTGTTGTCGGGGAAATCATCGTCCTTTCCTTGGCTGCTCGCCTATGTTGCCA
CCTGGATTCTGCGCGGGACGTCCTTCTGCTACGTCCCTTCGGCCCTCAATCCAGCGGACCTT
CCTTCCCGCGGCCTGCTGCCGGCTCTGCGGCCTCTTCCGCGTCTTCGCCTTCGCCCTCAGAC
GAGTCGGATCTCCCTTTGGGCCGCCTCCCCGCGAATTCATCGATACCGAGCGCTGCTCGAGA
GATCTGTGATAGCGGCCATCAAGCTGGCTGTGCCTTCTAGTTGCCAGCCATCTGTTGTTTGC
CCCTCCCCCGTGCCTTCCTTGACCCTGGAAGGTGCCACTCCCACTGTCCTTTCCTAATAAAA
TGAGGAAATTGCATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGACACGTGCGGACCG
AGCGGCCGCAGGAACCCCTAGTGATGGAGTTGGCCACTCCCTCTCTGCGCGCTCGCTCGCTC
ACTGAGGCCGGGCGACCAAAGGTCGCCCGACGCCCGGGCTTTGCCCGGGCGGCCTCAGTGAG
CGAGCGAGCGCGCAGCTGCCTGCAGGGGCGCCTGATGCGGTATTTTCTCCTTACGCATCTGT
GCGGTATTTCACACCGCATACGTCAAAGCAACCATAGTACGCGCCCTGTAGCGGCGCATTAA
GCGCGGCGGGTGTGGTGGTTACGCGCAGCGTGACCGCTACACTTGCCAGCGCCCTAGCGCCC
GCTCCTTTCGCTTTCTTCCCTTCCTTTCTCGCCACGTTCGCCGGCTTTCCCCGTCAAGCTCT
AAATCGGGGGCTCCCTTTAGGGTTCCGATTTAGTGCTTTACGGCACCTCGACCCCAAAAAAC
TTGATTTGGGTGATGGTTCACGTAGTGGGCCATCGCCCTGATAGACGGTTTTTCGCCCTTTG
ACGTTGGAGTCCACGTTCTTTAATAGTGGACTCTTGTTCCAAACTGGAACAACACTCAACCC
TATCTCGGGCTATTCTTTTGATTTATAAGGGATTTTGCCGATTTCGGCCTATTGGTTAAAAA
ATGAGCTGATTTAACAAAAATTTAACGCGAATTTTAACAAAATATTAACGTTTACAATTTTA
TGGTGCACTCTCAGTACAATCTGCTCTGATGCCGCATAGTTAAGCCAGCCCCGACACCCGCC
AACACCCGCTGACGCGCCCTGACGGGCTTGTCTGCTCCCGGCATCCGCTTACAGACAAGCTG
TGACCGTCTCCGGGAGCTGCATGTGTCAGAGGTTTTCACCGTCATCACCGAAACGCGCGAGA
CGAAAGGGCCTCGTGATACGCCTATTTTTATAGGTTAATGTCATGATAATAATGGTTTCTTA
GACGTCAGGTGGCACTTTTCGGGGAAATGTGCGCGGAACCCCTATTTGTTTATTTTTCTAAA
TACATTCAAATATGTATCCGCTCATGAGACAATAACCCTGATAAATGCTTCAATAATATTGA
AAAAGGAAGAGTATGAGTATTCAACATTTCCGTGTCGCCCTTATTCCCTTTTTTGCGGCATT
TTGCCTTCCTGTTTTTGCTCACCCAGAAACGCTGGTGAAAGTAAAAGATGCTGAAGATCAGT
TGGGTGCACGAGTGGGTTACATCGAACTGGATCTCAACAGCGGTAAGATCCTTGAGAGTTTT
CGCCCCGAAGAACGTTTTCCAATGATGAGCACTTTTAAAGTTCTGCTATGTGGCGCGGTATT
ATCCCGTATTGACGCCGGGCAAGAGCAACTCGGTCGCCGCATACACTATTCTCAGAATGACT
TGGTTGAGTACTCACCAGTCACAGAAAAGCATCTTACGGATGGCATGACAGTAAGAGAATTA
TGCAGTGCTGCCATAACCATGAGTGATAACACTGCGGCCAACTTACTTCTGACAACGATCGG
AGGACCGAAGGAGCTAACCGCTTTTTTGCACAACATGGGGGATCATGTAACTCGCCTTGATC
GTTGGGAACCGGAGCTGAATGAAGCCATACCAAACGACGAGCGTGACACCA

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 75. An exemplary nucleotide sequence for vector c.81.5 is set forth in SEQ ID NO: 75 (hGJB2 GRE5 underlined; human GJB2 coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTA
AGCTACTAACTACAACCACGAGATTATAGATGTTTGCTGATATTGTTCTCAGTTTGGTTATT
GTGTTGTTTATGAATGAAAGTAGTGTATGTTTGTGTGAATTTTTGTTTTTAATTTTTTATGA
GTGCCCTAACAAAGATTACAAATTGGGAATACAAACTCCAGAGCAATGGAGACAGTGACACT
TTTGTGGAGGGGTACATGTGGCTGTTCGGGTGGTTATTAACACAGGCTGCTGCCCCTGCCCT
GCAATGGGAATCCCCAGGGCATTGGAGGATTCAACCTCTTGCAGTTACCTCTTGTAAGACAG
CAGATGGCAGCAGAGAGAGGCTTTGCACATCCCTGCAGGTTCTAGTTTGCACAAAGGGCTTC
TGAGAGACCTATCAACCAATTATAACATCAAGTGGCAAAAAGAGTCCTTGATAAGTTATTTC
GCTTCTCAAAGAAACCGAAAACGCCAAACTAATCACTAGTCTTGTTTTTTTTTTTCCTGGCA
AAAGCCTGCTATCTTTCATGATTTAGCTTTCATGAAATTGTTCCTGAAGACCCCCAAAAGAA
ACAATTTCATGCCCCGAACTCTGTTCAGAGACTTTGCTGTGCCTGTCATGTCCAGCTTGCCA
TATCCTGTTTTGTAAAGTAGCCACCTTATATACACACCTGCTGTCTGCACTGTGACCTCCTT
TCAAAATCATCTTTGGTTCTTCAGAGGCCTGGAATAATGCTCTGCCCAGATGAAGATCTCCG
TAAATGTGTTTTTGAAATGGCTAATCAAATAATGGATACCCTTAGGTATTTTTGCAGAAACA
CTTGGCAGCCTTCCATAATATCCCTACTATGAAATGGAAACTTGTGAATGAGATGTGGCTTT
AATTAAGACCTCGAAGGGGACTTGGGGGGTTCGGGGCTTTCGGGGGCGGTCGGGGGTTCGCG
GACCCGGGAAGCTCTGAGGACCCAGAGGCCGGGCGCGCTCCGCCCGCGGCGCCGCCCCCTCC
GTAACTTTCCCAGTCTCCGAGGGAAGAGGCGGGGTGTGGGGTGCGGTTAAAAGGCGCCACGG
CGGGAGACAGGTGTTGCGGCCCCGCAGCGCCCGCGCGCTCCTCTCCCCGACTCGGAGCCCCT
CGGCGGCGCCCGGCCCAGGACCCGCCTAGGAGCGCAGGAGCCCCAGCGCAGAGACCCCAACG
CCGAGACCCCCGCCCCGGCCCCGCCGCGCTTCCTCCCGACGCAGAGCAAACCGCCCAGAGTA
GAAGCCATGGATTGGGGCACGCTGCAGACGATCCTGGGGGGTGTGAACAAACACTCCACCAG
CATTGGAAAGATCTGGCTCACCGTCCTCTTCATTTTTCGCATTATGATCCTCGTTGTGGCTG
CAAAGGAGGTGTGGGGAGATGAGCAGGCCGACTTTGTCTGCAACACCCTGCAGCCAGGCTGC
AAGAACGTGTGCTACGATCACTACTTCCCCATCTCCCACATCCGGCTATGGGCCCTGCAGCT
GATCTTCGTGTCCACGCCAGCGCTCCTAGTGGCCATGCACGTGGCCTACCGGAGACATGAGA
AGAAGAGGAAGTTCATCAAGGGGGAGATAAAGAGTGAATTTAAGGACATCGAGGAGATCAAA
ACCCAGAAGGTCCGCATCGAAGGCTCCCTGTGGTGGACCTACACAAGCAGCATCTTCTTCCG
GGTCATCTTCGAAGCCGCCTTCATGTACGTCTTCTATGTCATGTACGACGGCTTCTCCATGC
AGCGGCTGGTGAAGTGCAACGCCTGGCCTTGTCCCAACACTGTGGACTGCTTTGTGTCCCGG
CCCACGGAGAAGACTGTCTTCACAGTGTTCATGATTGCAGTGTCTGGAATTTGCATCCTGCT
GAATGTCACTGAATTGTGTTATTTGCTAATTAGATATTGTTCTGGGAAGTCAAAAAAGCCAG
TTTAAAGGCGCGCCACCCCTGCAGGGAATTCCGCATTGCCCAGTTGTTAGATTAAGAAATAG
ACAGCATGAGAGGGATGAGGCAACCCGTGCTCAGCTGTCAAGGCTCAGTCGCTAGCATTTCC
CAACACAAAGATTCTGACCTTAAATGCAACCATTTGAAACCCCTGTAGGCCTCAGGTGAAAC
TCCAGATGCCACAATGGAGCTCTGCTCCCCTAAAGCCTCAAAACAAAGGCCTAATTCTATGC
CTGTCTTAATTTTCTTTCACTTAAGTTAGTTCCACTGAGACCCCAGGCTGTTAGGGGTTATT
GGTGTAAGGTACTTTCATATTTTAAACAGAGGATATCGGCATTTGTTTCTTTCTCTGAGGAC
AAGAGAAAAAAGCCAGGTTCCACAGAGGACACAGAGAAGGTTTGGGTGTCCTCCTGGGGTTC
TTTTTGCCAACTTTCCCCACGTTAAAGGTGAACATTGGTTCTTTCATTTGCTTTGGAAGTTT
TAATCTCTAACAGTGGACAAAGTTACCAGTGCCTTAAACTCTGTTACACTTTTTGGAAGTGA
AAACTTTGTAGTATGATAGGTTATTTTGATGTAAAGATGTTCTGGATACCATTATATGTTCC
CCCTGTTTCAGAGGCTCAGATTGTAATATGTAAATGGTATGTCATTCGCTACTATGATTTAA
TTTGAAATATGGTCTTTTGGTTATGAATACTTTGCAGCACAGCTGAGAGGCTGTCTGTTGTA
TTCATTGTGGTCATAGCACCTAACAACATTGTAGCCTCAATCGAGTGAGACAGACTAGAAGT
TCCTAGTGATGGCTTATGATAGCAAATGGCCTCATGTCAAATATTTAGATGTAATTTTGTGT
AAGAAATACAGACTGGATGTACCACCAACTACTACCTGTAATGACAGGCCTGTCCAACACAT
CTCCCTTTTCCATGACTGTGGTAGCCAGCATCGGAAAGAACGCTGATTTAAAGAGGTCGCTT
GGGAATTTTATTGACACAGTACCATTTAATGGGGAGGACAAAATGGGGCAGGGGAGGGAGAA
GTTTCTGTCGTTAAAAACAGATTTGGAAAGACTGGACTCTAAAGTCTGTTGATTAAAGATGA
GCTTTGTCTACTTCAAAAGTTTGTTTGCTTACCCCTTCAGCCTCCAATTTTTTAAGTGAAAA
TATAGCTAATAACATGTGAAAAGAATAGAAGCTAAGGTTTAGATAAATATTGAGCAGATCTA
TAGGAAGATTGAACCTGAATATTGCCATTATGCTTGACATGGTTTCCAAAAAATGGTACTCC
ACATATTTCAGTGAGGGTAAGTATTTTCCTGTTGTCAAGAATAGCATTGTAAAAGCATTTTG
TAATAATAAAGAATAGCTTTAATGATATGCTTGTAACTAAAATAATTTTGTAATGTATCAAA
TACATTTAAAACATTAAAATATAATCTCTATAATAATTTAAAATCTAATATGGTTTTAATAG
AACAGCGATATCAAGCTTATCGATAATCAACCTCTGGATTACAAAATTTGTGAAAGATTGAC
TGGTATTCTTAACTATGTTGCTCCTTTTACGCTATGTGGATACGCTGCTTTAATGCCTTTGT
ATCATGCTATTGCTTCCCGTATGGCTTTCATTTTCTCCTCCTTGTATAAATCCTGGTTGCTG
TCTCTTTATGAGGAGTTGTGGCCCGTTGTCAGGCAACGTGGCGTGGTGTGCACTGTGTTTGC
TGACGCAACCCCCACTGGTTGGGGCATTGCCACCACCTGTCAGCTCCTTTCCGGGACTTTCG
CTTTCCCCCTCCCTATTGCCACGGCGGAACTCATCGCCGCCTGCCTTGCCCGCTGCTGGACA
GGGGCTCGGCTGTTGGGCACTGACAATTCCGTGGTGTTGTCGGGGAAATCATCGTCCTTTCC
TTGGCTGCTCGCCTATGTTGCCACCTGGATTCTGCGCGGGACGTCCTTCTGCTACGTCCCTT
CGGCCCTCAATCCAGCGGACCTTCCTTCCCGCGGCCTGCTGCCGGCTCTGCGGCCTCTTCCG
CGTCTTCGCCTTCGCCCTCAGACGAGTCGGATCTCCCTTTGGGCCGCCTCCCCGCGAATTCA
TCGATACCGAGCGCTGCTCGAGAGATCTGTGATAGCGGCCATCAAGCTGGCTGTGCCTTCTA
GTTGCCAGCCATCTGTTGTTTGCCCCTCCCCCGTGCCTTCCTTGACCCTGGAAGGTGCCACT
CCCACTGTCCTTTCCTAATAAAATGAGGAAATTGCATCGCATTGTCTGAGTAGGTGTCATTC
TATTCTGGGGGGTGGGGTGGGGCAGGACAGCAAGGGGGAGGATTGGGAAGACAATAGCAGGC
ATGCTGGGGACACGTGCGGACCGAGCGGCCGCAGGAACCCCTAGTGATGGAGTTGGCCACTC
CCTCTCTGCGCGCTCGCTCGCTCACTGAGGCCGGGCGACCAAAGGTCGCCCGACGCCCGGGC
TTTGCCCGGGCGGCCTCAGTGAGCGAGCGAGCGCGCAGCTGCCTGCAGGGGCGCCTGATGCG
GTATTTTCTCCTTACGCATCTGTGCGGTATTTCACACCGCATACGTCAAAGCAACCATAGTA
CGCGCCCTGTAGCGGCGCATTAAGCGCGGCGGGTGTGGTGGTTACGCGCAGCGTGACCGCTA
CACTTGCCAGCGCCCTAGCGCCCGCTCCTTTCGCTTTCTTCCCTTCCTTTCTCGCCACGTTC
GCCGGCTTTCCCCGTCAAGCTCTAAATCGGGGGCTCCCTTTAGGGTTCCGATTTAGTGCTTT
ACGGCACCTCGACCCCAAAAAACTTGATTTGGGTGATGGTTCACGTAGTGGGCCATCGCCCT
GATAGACGGTTTTTCGCCCTTTGACGTTGGAGTCCACGTTCTTTAATAGTGGACTCTTGTTC
CAAACTGGAACAACACTCAACCCTATCTCGGGCTATTCTTTTGATTTATAAGGGATTTTGCC
GATTTCGGCCTATTGGTTAAAAAATGAGCTGATTTAACAAAAATTTAACGCGAATTTTAACA
AAATATTAACGTTTACAATTTTATGGTGCACTCTCAGTACAATCTGCTCTGATGCCGCATAG
TTAAGCCAGCCCCGACACCCGCCAACACCCGCTGACGCGCCCTGACGGGCTTGTCTGCTCCC
GGCATCCGCTTACAGACAAGCTGTGACCGTCTCCGGGAGCTGCATGTGTCAGAGGTTTTCAC
CGTCATCACCGAAACGCGCGAGACGAAAGGGCCTCGTGATACGCCTATTTTTATAGGTTAAT
GTCATGATAATAATGGTTTCTTAGACGTCAGGTGGCACTTTTCGGGGAAATGTGCGCGGAAC
CCCTATTTGTTTATTTTTCTAAATACATTCAAATATGTATCCGCTCATGAGACAATAACCCT
GATAAATGCTTCAATAATATTGAAAAAGGAAGAGTATGAGTATTCAACATTTCCGTGTCGCC
CTTATTCCCTTTTTTGCGGCATTTTGCCTTCCTGTTTTTGCTCACCCAGAAACGCTGGTGAA
AGTAAAAGATGCTGAAGATCAGTTGGGTGCACGAGTGGGTTACATCGAACTGGATCTCAACA
GCGGTAAGATCCTTGAGAGTTTTCGCCCCGAAGAACGTTTTCCAATGATGAGCACTTTTAAA
GTTCTGCTATGTGGCGCGGTATTATCCCGTATTGACGCCGGGCAAGAGCAACTCGGTCGCCG
CATACACTATTCTCAGAATGACTTGGTTGAGTACTCACCAGTCACAGAAAAGCATCTTACGG
ATGGCATGACAGTAAGAGAATTATGCAGTGCTGCCATAACCATGAGTGATAACACTGCGGCC
AACTTACTTCTGACAACGATCGGAGGACCGAAGGAGCTAACCGCTTTTTTGCACAACATGGG
GGATCATGTAACTCGCCTTGATCGTTGGGAACCGGAGCTGAATGAAGCCATACCAAACGACG
AGCGTGACACCA

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 76. An exemplary nucleotide sequence for vector c.81.5 is set forth in SEQ ID NO: 76 (hGJB2 GRE5 underlined; mouse GJB2 coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTA
AGCTACTAACTACAACCACGAGATTATAGATGTTTGCTGATATTGTTCTCAGTTTGGTTATT
GTGTTGTTTATGAATGAAAGTAGTGTATGTTTGTGTGAATTTTTGTTTTTAATTTTTTATGA
GTGCCCTAACAAAGATTACAAATTGGGAATACAAACTCCAGAGCAATGGAGACAGTGACACT
TTTGTGGAGGGGTACATGTGGCTGTTCGGGTGGTTATTAACACAGGCTGCTGCCCCTGCCCT
GCAATGGGAATCCCCAGGGCATTGGAGGATTCAACCTCTTGCAGTTACCTCTTGTAAGACAG
CAGATGGCAGCAGAGAGAGGCTTTGCACATCCCTGCAGGTTCTAGTTTGCACAAAGGGCTTC
TGAGAGACCTATCAACCAATTATAACATCAAGTGGCAAAAAGAGTCCTTGATAAGTTATTTC
GCTTCTCAAAGAAACCGAAAACGCCAAACTAATCACTAGTCTTGTTTTTTTTTTTCCTGGCA
AAAGCCTGCTATCTTTCATGATTTAGCTTTCATGAAATTGTTCCTGAAGACCCCCAAAAGAA
ACAATTTCATGCCCCGAACTCTGTTCAGAGACTTTGCTGTGCCTGTCATGTCCAGCTTGCCA
TATCCTGTTTTGTAAAGTAGCCACCTTATATACACACCTGCTGTCTGCACTGTGACCTCCTT
TCAAAATCATCTTTGGTTCTTCAGAGGCCTGGAATAATGCTCTGCCCAGATGAAGATCTCCG
TAAATGTGTTTTTGAAATGGCTAATCAAATAATGGATACCCTTAGGTATTTTTGCAGAAACA
CTTGGCAGCCTTCCATAATATCCCTACTATGAAATGGAAACTTGTGAATGAGATGTGGCTTT
AATTAAGACCTCGAAGGGGACTTGGGGGGTTCGGGGCTTTCGGGGGCGGTCGGGGGTTCGCG
GACCCGGGAAGCTCTGAGGACCCAGAGGCCGGGCGCGCTCCGCCCGCGGCGCCGCCCCCTCC
GTAACTTTCCCAGTCTCCGAGGGAAGAGGCGGGGTGTGGGGTGCGGTTAAAAGGCGCCACGG
CGGGAGACAGGTGTTGCGGCCCCGCAGCGCCCGCGCGCTCCTCTCCCCGACTCGGAGCCCCT
CGGCGGCGCCCGGCCCAGGACCCGCCTAGGAGCGCAGGAGCCCCAGCGCAGAGACCCCAACG
CCGAGACCCCCGCCCCGGCCCCGCCGCGCTTCCTCCCGACGCAGAGCAAACCGCCCAGAGTA
GAAGCCATGGATTGGGGCACACTCCAGAGCATCCTCGGGGGTGTCAACAAACACTCCACCAG
CATTGGAAAGATCTGGCTCACGGTCCTCTTCATCTTCCGCATCATGATCCTCGTGGTGGCTG
CAAAGGAGGTGTGGGGAGATGAGCAAGCCGATTTTGTCTGCAACACGCTCCAGCCTGGCTGC
AAGAATGTATGCTACGACCACCACTTCCCCATCTCTCACATCCGGCTCTGGGCTCTGCAGCT
GATCATGGTGTCCACGCCAGCCCTCCTGGTAGCTATGCATGTGGCCTACCGGAGACATGAAA
AGAAACGGAAGTTCATGAAGGGAGAGATAAAGAACGAGTTTAAGGACATCGAAGAGATCAAA
ACCCAGAAGGTCCGTATCGAAGGGTCCCTGTGGTGGACCTACACCACCAGCATCTTCTTCCG
GGTCATCTTTGAAGCCGTCTTCATGTACGTCTTTTACATCATGTACAATGGCTTCTTCATGC
AACGTCTGGTGAAATGCAACGCTTGGCCCTGCCCCAATACAGTGGACTGCTTCATTTCCAGG
CCCACAGAAAAGACTGTCTTCACCGTGTTTATGATTTCTGTGTCTGGAATTTGCATTCTGCT
AAATATCACAGAGCTGTGCTATTTGTTCGTTAGGTATTGCTCAGGAAAGTCCAAAAGACCAG
TCTACCCATACGATGTTCCAGATTACGCTTAAAGGCGCGCCACCCCTGCAGGGAATTCCGCA
TTGCCCAGTTGTTAGATTAAGAAATAGACAGCATGAGAGGGATGAGGCAACCCGTGCTCAGC
TGTCAAGGCTCAGTCGCTAGCATTTCCCAACACAAAGATTCTGACCTTAAATGCAACCATTT
GAAACCCCTGTAGGCCTCAGGTGAAACTCCAGATGCCACAATGGAGCTCTGCTCCCCTAAAG
CCTCAAAACAAAGGCCTAATTCTATGCCTGTCTTAATTTTCTTTCACTTAAGTTAGTTCCAC
TGAGACCCCAGGCTGTTAGGGGTTATTGGTGTAAGGTACTTTCATATTTTAAACAGAGGATA
TCGGCATTTGTTTCTTTCTCTGAGGACAAGAGAAAAAAGCCAGGTTCCACAGAGGACACAGA
GAAGGTTTGGGTGTCCTCCTGGGGTTCTTTTTGCCAACTTTCCCCACGTTAAAGGTGAACAT
TGGTTCTTTCATTTGCTTTGGAAGTTTTAATCTCTAACAGTGGACAAAGTTACCAGTGCCTT
AAACTCTGTTACACTTTTTGGAAGTGAAAACTTTGTAGTATGATAGGTTATTTTGATGTAAA
GATGTTCTGGATACCATTATATGTTCCCCCTGTTTCAGAGGCTCAGATTGTAATATGTAAAT
GGTATGTCATTCGCTACTATGATTTAATTTGAAATATGGTCTTTTGGTTATGAATACTTTGC
AGCACAGCTGAGAGGCTGTCTGTTGTATTCATTGTGGTCATAGCACCTAACAACATTGTAGC
CTCAATCGAGTGAGACAGACTAGAAGTTCCTAGTGATGGCTTATGATAGCAAATGGCCTCAT
GTCAAATATTTAGATGTAATTTTGTGTAAGAAATACAGACTGGATGTACCACCAACTACTAC
CTGTAATGACAGGCCTGTCCAACACATCTCCCTTTTCCATGACTGTGGTAGCCAGCATCGGA
AAGAACGCTGATTTAAAGAGGTCGCTTGGGAATTTTATTGACACAGTACCATTTAATGGGGA
GGACAAAATGGGGCAGGGGAGGGAGAAGTTTCTGTCGTTAAAAACAGATTTGGAAAGACTGG
ACTCTAAAGTCTGTTGATTAAAGATGAGCTTTGTCTACTTCAAAAGTTTGTTTGCTTACCCC
TTCAGCCTCCAATTTTTTAAGTGAAAATATAGCTAATAACATGTGAAAAGAATAGAAGCTAA
GGTTTAGATAAATATTGAGCAGATCTATAGGAAGATTGAACCTGAATATTGCCATTATGCTT
GACATGGTTTCCAAAAAATGGTACTCCACATATTTCAGTGAGGGTAAGTATTTTCCTGTTGT
CAAGAATAGCATTGTAAAAGCATTTTGTAATAATAAAGAATAGCTTTAATGATATGCTTGTA
ACTAAAATAATTTTGTAATGTATCAAATACATTTAAAACATTAAAATATAATCTCTATAATA
ATTTAAAATCTAATATGGTTTTAATAGAACAGCGATATCAAGCTTATCGATAATCAACCTCT
GGATTACAAAATTTGTGAAAGATTGACTGGTATTCTTAACTATGTTGCTCCTTTTACGCTAT
GTGGATACGCTGCTTTAATGCCTTTGTATCATGCTATTGCTTCCCGTATGGCTTTCATTTTC
TCCTCCTTGTATAAATCCTGGTTGCTGTCTCTTTATGAGGAGTTGTGGCCCGTTGTCAGGCA
ACGTGGCGTGGTGTGCACTGTGTTTGCTGACGCAACCCCCACTGGTTGGGGCATTGCCACCA
CCTGTCAGCTCCTTTCCGGGACTTTCGCTTTCCCCCTCCCTATTGCCACGGCGGAACTCATC
GCCGCCTGCCTTGCCCGCTGCTGGACAGGGGCTCGGCTGTTGGGCACTGACAATTCCGTGGT
GTTGTCGGGGAAATCATCGTCCTTTCCTTGGCTGCTCGCCTATGTTGCCACCTGGATTCTGC
GCGGGACGTCCTTCTGCTACGTCCCTTCGGCCCTCAATCCAGCGGACCTTCCTTCCCGCGGC
CTGCTGCCGGCTCTGCGGCCTCTTCCGCGTCTTCGCCTTCGCCCTCAGACGAGTCGGATCTC
CCTTTGGGCCGCCTCCCCGCGAATTCATCGATACCGAGCGCTGCTCGAGAGATCTGTGATAG
CGGCCATCAAGCTGGCTGTGCCTTCTAGTTGCCAGCCATCTGTTGTTTGCCCCTCCCCCGTG
CCTTCCTTGACCCTGGAAGGTGCCACTCCCACTGTCCTTTCCTAATAAAATGAGGAAATTGC
ATCGCATTGTCTGAGTAGGTGTCATTCTATTCTGGGGGGTGGGGTGGGGCAGGACAGCAAGG
GGGAGGATTGGGAAGACAATAGCAGGCATGCTGGGGACACGTGCGGACCGAGCGGCCGCAGG
AACCCCTAGTGATGGAGTTGGCCACTCCCTCTCTGCGCGCTCGCTCGCTCACTGAGGCCGGG
CGACCAAAGGTCGCCCGACGCCCGGGCTTTGCCCGGGCGGCCTCAGTGAGCGAGCGAGCGCG
CAGCTGCCTGCAGGGGCGCCTGATGCGGTATTTTCTCCTTACGCATCTGTGCGGTATTTCAC
ACCGCATACGTCAAAGCAACCATAGTACGCGCCCTGTAGCGGCGCATTAAGCGCGGCGGGTG
TGGTGGTTACGCGCAGCGTGACCGCTACACTTGCCAGCGCCCTAGCGCCCGCTCCTTTCGCT
TTCTTCCCTTCCTTTCTCGCCACGTTCGCCGGCTTTCCCCGTCAAGCTCTAAATCGGGGGCT
CCCTTTAGGGTTCCGATTTAGTGCTTTACGGCACCTCGACCCCAAAAAACTTGATTTGGGTG
ATGGTTCACGTAGTGGGCCATCGCCCTGATAGACGGTTTTTCGCCCTTTGACGTTGGAGTCC
ACGTTCTTTAATAGTGGACTCTTGTTCCAAACTGGAACAACACTCAACCCTATCTCGGGCTA
TTCTTTTGATTTATAAGGGATTTTGCCGATTTCGGCCTATTGGTTAAAAAATGAGCTGATTT
AACAAAAATTTAACGCGAATTTTAACAAAATATTAACGTTTACAATTTTATGGTGCACTCTC
AGTACAATCTGCTCTGATGCCGCATAGTTAAGCCAGCCCCGACACCCGCCAACACCCGCTGA
CGCGCCCTGACGGGCTTGTCTGCTCCCGGCATCCGCTTACAGACAAGCTGTGACCGTCTCCG
GGAGCTGCATGTGTCAGAGGTTTTCACCGTCATCACCGAAACGCGCGAGACGAAAGGGCCTC
GTGATACGCCTATTTTTATAGGTTAATGTCATGATAATAATGGTTTCTTAGACGTCAGGTGG
CACTTTTCGGGGAAATGTGCGCGGAACCCCTATTTGTTTATTTTTCTAAATACATTCAAATA
TGTATCCGCTCATGAGACAATAACCCTGATAAATGCTTCAATAATATTGAAAAAGGAAGAGT
ATGAGTATTCAACATTTCCGTGTCGCCCTTATTCCCTTTTTTGCGGCATTTTGCCTTCCTGT
TTTTGCTCACCCAGAAACGCTGGTGAAAGTAAAAGATGCTGAAGATCAGTTGGGTGCACGAG
TGGGTTACATCGAACTGGATCTCAACAGCGGTAAGATCCTTGAGAGTTTTCGCCCCGAAGAA
CGTTTTCCAATGATGAGCACTTTTAAAGTTCTGCTATGTGGCGCGGTATTATCCCGTATTGA
CGCCGGGCAAGAGCAACTCGGTCGCCGCATACACTATTCTCAGAATGACTTGGTTGAGTACT
CACCAGTCACAGAAAAGCATCTTACGGATGGCATGACAGTAAGAGAATTATGCAGTGCTGCC
ATAACCATGAGTGATAACACTGCGGCCAACTTACTTCTGACAACGATCGGAGGACCGAAGGA
GCTAACCGCTTTTTTGCACAACATGGGGGATCATGTAACTCGCCTTGATCGTTGGGAACCGG
AGCTGAATGAAGCCATACCAAACGACGAGCGTGACACCA

In some embodiments, an AAV vector described herein comprises an AAV 5′ ITR, a GJB2 GRE enhancer (hGJB2 GRE7), a GJB2 basal promoter, a GJB2 exon 1 5′ UTR, Kozak sequence, nucleotide sequence encoding a gene product (e.g., GJB2 or GFP), a GJB2 exon 2 3′ UTR, a WPRE, a bovine growth hormone poly A signal, and an AAV 3′ ITR (e.g., vector c.81.7).

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 51. An exemplary nucleotide sequence for vector c.81.7 is set forth in SEQ ID NO: 51 (hGJB2 GRE7 underlined; eGFP coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTC
TCAGCTGGAGTGACGCACCTCATCCATGCGGGCCTGGCGTCTGGAAGGTGGCTGGGTCTCTC
GGGCTTGAGCACCATCATCTTAGCTCCAACATGTCATTATTCCTTCCTCACTGAGGACTTTT
CTGCTTCCTAATTGGTTGTTGAAGATGAGGCCCCCATGCTCTTTTAAGAAAACCTGTTGTGC
CCCAGGCTTGGCTGTGATGGGCACTGACTCATACAGAAGTAGAAAGGCCTGCTGAGTCATCA
ACACTCGTGCGACGCCCTCGCATTTTCATTAATGATGGCCTCCCTGCCACACGTGAATCACT
CCAGCCCGAGATCTGAAACCAGGACACACCCCAGGGGCGAGGTGACGCTGAGTGAGCCCAGC
TGTGTCCCTTTCATGAGAACTCAGAGCACAGGGCTCTGTGTGCATGGCCGTCCCCTCCAGAG
AGGAGGAAGTAAATGCCGGGATTAGTGGAAGATCATTTCCTTCTATTTGCCTTGGCTTACGT
CTTTCAGAATTCAAACACGTGCACTGTTGACCCTGCAATGGTGGAGTTTTTGGATTTTCCTT
CAGTCCGATTGCTAAAATACTTCCCTCTCATGTGAGCTGTTGTGAAAGTCATCAGCCAGATA
CCATTCTAAAAACAAAGAATGTGCTTCTCGTATGTTGCATGCTGGTTACTGAAATATTAGGG
AATTACATAAAGGTTTTCTGGGGCACATATTCAAGCTGAATGATAAAATTGAAGGTCACACA
AAGCTAAGGTCTTTCAAATCCTGACCCAATTAGCTCTCTGTTAGCTCTCTGACTTTGGACAA
GCTGTCTGGTCCTCTGAAGCATACTTTGTTCGCCCTGGGTAGGGGCCCTCTGTTTTAACAGC
GTTTGGCATTAATTAAGACCTCGAAGGGGACTTGGGGGGTTCGGGGCTTTCGGGGGCGGTCG
GGGGTTCGCGGACCCGGGAAGCTCTGAGGACCCAGAGGCCGGGCGCGCTCCGCCCGCGGCGC
CGCCCCCTCCGTAACTTTCCCAGTCTCCGAGGGAAGAGGCGGGGTGTGGGGTGCGGTTAAAA
GGCGCCACGGCGGGAGACAGGTGTTGCGGCCCCGCAGCGCCCGCGCGCTCCTCTCCCCGACT
CGGAGCCCCTCGGCGGCGCCCGGCCCAGGACCCGCCTAGGAGCGCAGGAGCCCCAGCGCAGA
GACCCCAACGCCGAGACCCCCGCCCCGGCCCCGCCGCGCTTCCTCCCGACGCAGAGCAAACC
GCCCAGAGTAGAAGCCATGGTGAGCAAGGGCGAGGAGCTGTTCACCGGGGTGGTGCCCATCC
TGGTCGAGCTGGACGGCGACGTAAACGGCCACAAGTTCAGCGTGTCCGGCGAGGGCGAGGGC
GATGCCACCTACGGCAAGCTGACCCTGAAGTTCATCTGCACCACCGGCAAGCTGCCCGTGCC
CTGGCCCACCCTCGTGACCACCCTGACCTACGGCGTGCAGTGCTTCAGCCGCTACCCCGACC
ACATGAAGCAGCACGACTTCTTCAAGTCCGCCATGCCCGAAGGCTACGTCCAGGAGCGCACC
ATCTTCTTCAAGGACGACGGCAACTACAAGACCCGCGCCGAGGTGAAGTTCGAGGGCGACAC
CCTGGTGAACCGCATCGAGCTGAAGGGCATCGACTTCAAGGAGGACGGCAACATCCTGGGGC
ACAAGCTGGAGTACAACTACAACAGCCACAACGTCTATATCATGGCCGACAAGCAGAAGAAC
GGCATCAAGGTGAACTTCAAGATCCGCCACAACATCGAGGACGGCAGCGTGCAGCTCGCCGA
CCACTACCAGCAGAACACCCCCATCGGCGACGGCCCCGTGCTGCTGCCCGACAACCACTACC
TGAGCACCCAGTCCGCCCTGAGCAAAGACCCCAACGAGAAGCGCGATCACATGGTCCTGCTG
GAGTTCGTGACCGCCGCCGGGATCACTCTCGGCATGGACGAGCTGTACAAGTAAAGGCGCGC
CACCCCTGCAGGGAATTCCGCATTGCCCAGTTGTTAGATTAAGAAATAGACAGCATGAGAGG
GATGAGGCAACCCGTGCTCAGCTGTCAAGGCTCAGTCGCTAGCATTTCCCAACACAAAGATT
CTGACCTTAAATGCAACCATTTGAAACCCCTGTAGGCCTCAGGTGAAACTCCAGATGCCACA
ATGGAGCTCTGCTCCCCTAAAGCCTCAAAACAAAGGCCTAATTCTATGCCTGTCTTAATTTT
CTTTCACTTAAGTTAGTTCCACTGAGACCCCAGGCTGTTAGGGGTTATTGGTGTAAGGTACT
TTCATATTTTAAACAGAGGATATCGGCATTTGTTTCTTTCTCTGAGGACAAGAGAAAAAAGC
CAGGTTCCACAGAGGACACAGAGAAGGTTTGGGTGTCCTCCTGGGGTTCTTTTTGCCAACTT
TCCCCACGTTAAAGGTGAACATTGGTTCTTTCATTTGCTTTGGAAGTTTTAATCTCTAACAG
TGGACAAAGTTACCAGTGCCTTAAACTCTGTTACACTTTTTGGAAGTGAAAACTTTGTAGTA
TGATAGGTTATTTTGATGTAAAGATGTTCTGGATACCATTATATGTTCCCCCTGTTTCAGAG
GCTCAGATTGTAATATGTAAATGGTATGTCATTCGCTACTATGATTTAATTTGAAATATGGT
CTTTTGGTTATGAATACTTTGCAGCACAGCTGAGAGGCTGTCTGTTGTATTCATTGTGGTCA
TAGCACCTAACAACATTGTAGCCTCAATCGAGTGAGACAGACTAGAAGTTCCTAGTGATGGC
TTATGATAGCAAATGGCCTCATGTCAAATATTTAGATGTAATTTTGTGTAAGAAATACAGAC
TGGATGTACCACCAACTACTACCTGTAATGACAGGCCTGTCCAACACATCTCCCTTTTCCAT
GACTGTGGTAGCCAGCATCGGAAAGAACGCTGATTTAAAGAGGTCGCTTGGGAATTTTATTG
ACACAGTACCATTTAATGGGGAGGACAAAATGGGGCAGGGGAGGGAGAAGTTTCTGTCGTTA
AAAACAGATTTGGAAAGACTGGACTCTAAAGTCTGTTGATTAAAGATGAGCTTTGTCTACTT
CAAAAGTTTGTTTGCTTACCCCTTCAGCCTCCAATTTTTTAAGTGAAAATATAGCTAATAAC
ATGTGAAAAGAATAGAAGCTAAGGTTTAGATAAATATTGAGCAGATCTATAGGAAGATTGAA
CCTGAATATTGCCATTATGCTTGACATGGTTTCCAAAAAATGGTACTCCACATATTTCAGTG
AGGGTAAGTATTTTCCTGTTGTCAAGAATAGCATTGTAAAAGCATTTTGTAATAATAAAGAA
TAGCTTTAATGATATGCTTGTAACTAAAATAATTTTGTAATGTATCAAATACATTTAAAACA
TTAAAATATAATCTCTATAATAATTTAAAATCTAATATGGTTTTAATAGAACAGCGATATCA
AGCTTATCGATAATCAACCTCTGGATTACAAAATTTGTGAAAGATTGACTGGTATTCTTAAC
TATGTTGCTCCTTTTACGCTATGTGGATACGCTGCTTTAATGCCTTTGTATCATGCTATTGC
TTCCCGTATGGCTTTCATTTTCTCCTCCTTGTATAAATCCTGGTTGCTGTCTCTTTATGAGG
AGTTGTGGCCCGTTGTCAGGCAACGTGGCGTGGTGTGCACTGTGTTTGCTGACGCAACCCCC
ACTGGTTGGGGCATTGCCACCACCTGTCAGCTCCTTTCCGGGACTTTCGCTTTCCCCCTCCC
TATTGCCACGGCGGAACTCATCGCCGCCTGCCTTGCCCGCTGCTGGACAGGGGCTCGGCTGT
TGGGCACTGACAATTCCGTGGTGTTGTCGGGGAAATCATCGTCCTTTCCTTGGCTGCTCGCC
TATGTTGCCACCTGGATTCTGCGCGGGACGTCCTTCTGCTACGTCCCTTCGGCCCTCAATCC
AGCGGACCTTCCTTCCCGCGGCCTGCTGCCGGCTCTGCGGCCTCTTCCGCGTCTTCGCCTTC
GCCCTCAGACGAGTCGGATCTCCCTTTGGGCCGCCTCCCCGCGAATTCATCGATACCGAGCG
CTGCTCGAGAGATCTGTGATAGCGGCCATCAAGCTGGCTGTGCCTTCTAGTTGCCAGCCATC
TGTTGTTTGCCCCTCCCCCGTGCCTTCCTTGACCCTGGAAGGTGCCACTCCCACTGTCCTTT
CCTAATAAAATGAGGAAATTGCATCGCATTGTCTGAGTAGGTGTCATTCTATTCTGGGGGGT
GGGGTGGGGCAGGACAGCAAGGGGGAGGATTGGGAAGACAATAGCAGGCATGCTGGGGACAC
GTGCGGACCGAGCGGCCGCAGGAACCCCTAGTGATGGAGTTGGCCACTCCCTCTCTGCGCGC
TCGCTCGCTCACTGAGGCCGGGCGACCAAAGGTCGCCCGACGCCCGGGCTTTGCCCGGGCGG
CCTCAGTGAGCGAGCGAGCGCGCAGCTGCCTGCAGGGGCGCCTGATGCGGTATTTTCTCCTT
ACGCATCTGTGCGGTATTTCACACCGCATACGTCAAAGCAACCATAGTACGCGCCCTGTAGC
GGCGCATTAAGCGCGGCGGGTGTGGTGGTTACGCGCAGCGTGACCGCTACACTTGCCAGCGC
CCTAGCGCCCGCTCCTTTCGCTTTCTTCCCTTCCTTTCTCGCCACGTTCGCCGGCTTTCCCC
GTCAAGCTCTAAATCGGGGGCTCCCTTTAGGGTTCCGATTTAGTGCTTTACGGCACCTCGAC
CCCAAAAAACTTGATTTGGGTGATGGTTCACGTAGTGGGCCATCGCCCTGATAGACGGTTTT
TCGCCCTTTGACGTTGGAGTCCACGTTCTTTAATAGTGGACTCTTGTTCCAAACTGGAACAA
CACTCAACCCTATCTCGGGCTATTCTTTTGATTTATAAGGGATTTTGCCGATTTCGGCCTAT
TGGTTAAAAAATGAGCTGATTTAACAAAAATTTAACGCGAATTTTAACAAAATATTAACGTT
TACAATTTTATGGTGCACTCTCAGTACAATCTGCTCTGATGCCGCATAGTTAAGCCAGCCCC
GACACCCGCCAACACCCGCTGACGCGCCCTGACGGGCTTGTCTGCTCCCGGCATCCGCTTAC
AGACAAGCTGTGACCGTCTCCGGGAGCTGCATGTGTCAGAGGTTTTCACCGTCATCACCGAA
ACGCGCGAGACGAAAGGGCCTCGTGATACGCCTATTTTTATAGGTTAATGTCATGATAATAA
TGGTTTCTTAGACGTCAGGTGGCACTTTTCGGGGAAATGTGCGCGGAACCCCTATTTGTTTA
TTTTTCTAAATACATTCAAATATGTATCCGCTCATGAGACAATAACCCTGATAAATGCTTCA
ATAATATTGAAAAAGGAAGAGTATGAGTATTCAACATTTCCGTGTCGCCCTTATTCCCTTTT
TTGCGGCATTTTGCCTTCCTGTTTTTGCTCACCCAGAAACGCTGGTGAAAGTAAAAGATGCT
GAAGATCAGTTGGGTGCACGAGTGGGTTACATCGAACTGGATCTCAACAGCGGTAAGATCCT
TGAGAGTTTTCGCCCCGAAGAACGTTTTCCAATGATGAGCACTTTTAAAGTTCTGCTATGTG
GCGCGGTATTATCCCGTATTGACGCCGGGCAAGAGCAACTCGGTCGCCGCATACACTATTCT
CAGAATGACTTGGTTGAGTACTCACCAGTCACAGAAAAGCATCTTACGGATGGCATGACAGT
AAGAGAATTATGCAGTGCTGCCATAACCATGAGTGATAACACTGCGGCCAACTTACTTCTGA
CAACGATCGGAGGACCGAAGGAGCTAACCGCTTTTTTGCACAACATGGGGGATCATGTAACT
CGCCTTGATCGTTGGGAACCGGAGCTGAATGAAGCCATACCAAACGACGAGCGTGACACCA

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 77. An exemplary nucleotide sequence for vector c.81.7 is set forth in SEQ ID NO: 77 (hGJB2 GRE7 underlined; human GJB2 coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTC
TCAGCTGGAGTGACGCACCTCATCCATGCGGGCCTGGCGTCTGGAAGGTGGCTGGGTCTCTC
GGGCTTGAGCACCATCATCTTAGCTCCAACATGTCATTATTCCTTCCTCACTGAGGACTTTT
CTGCTTCCTAATTGGTTGTTGAAGATGAGGCCCCCATGCTCTTTTAAGAAAACCTGTTGTGC
CCCAGGCTTGGCTGTGATGGGCACTGACTCATACAGAAGTAGAAAGGCCTGCTGAGTCATCA
ACACTCGTGCGACGCCCTCGCATTTTCATTAATGATGGCCTCCCTGCCACACGTGAATCACT
CCAGCCCGAGATCTGAAACCAGGACACACCCCAGGGGCGAGGTGACGCTGAGTGAGCCCAGC
TGTGTCCCTTTCATGAGAACTCAGAGCACAGGGCTCTGTGTGCATGGCCGTCCCCTCCAGAG
AGGAGGAAGTAAATGCCGGGATTAGTGGAAGATCATTTCCTTCTATTTGCCTTGGCTTACGT
CTTTCAGAATTCAAACACGTGCACTGTTGACCCTGCAATGGTGGAGTTTTTGGATTTTCCTT
CAGTCCGATTGCTAAAATACTTCCCTCTCATGTGAGCTGTTGTGAAAGTCATCAGCCAGATA
CCATTCTAAAAACAAAGAATGTGCTTCTCGTATGTTGCATGCTGGTTACTGAAATATTAGGG
AATTACATAAAGGTTTTCTGGGGCACATATTCAAGCTGAATGATAAAATTGAAGGTCACACA
AAGCTAAGGTCTTTCAAATCCTGACCCAATTAGCTCTCTGTTAGCTCTCTGACTTTGGACAA
GCTGTCTGGTCCTCTGAAGCATACTTTGTTCGCCCTGGGTAGGGGCCCTCTGTTTTAACAGC
GTTTGGCATTAATTAAGACCTCGAAGGGGACTTGGGGGGTTCGGGGCTTTCGGGGGCGGTCG
GGGGTTCGCGGACCCGGGAAGCTCTGAGGACCCAGAGGCCGGGCGCGCTCCGCCCGCGGCGC
CGCCCCCTCCGTAACTTTCCCAGTCTCCGAGGGAAGAGGCGGGGTGTGGGGTGCGGTTAAAA
GGCGCCACGGCGGGAGACAGGTGTTGCGGCCCCGCAGCGCCCGCGCGCTCCTCTCCCCGACT
CGGAGCCCCTCGGCGGCGCCCGGCCCAGGACCCGCCTAGGAGCGCAGGAGCCCCAGCGCAGA
GACCCCAACGCCGAGACCCCCGCCCCGGCCCCGCCGCGCTTCCTCCCGACGCAGAGCAAACC
GCCCAGAGTAGAAGCCATGGATTGGGGCACGCTGCAGACGATCCTGGGGGGTGTGAACAAAC
ACTCCACCAGCATTGGAAAGATCTGGCTCACCGTCCTCTTCATTTTTCGCATTATGATCCTC
GTTGTGGCTGCAAAGGAGGTGTGGGGAGATGAGCAGGCCGACTTTGTCTGCAACACCCTGCA
GCCAGGCTGCAAGAACGTGTGCTACGATCACTACTTCCCCATCTCCCACATCCGGCTATGGG
CCCTGCAGCTGATCTTCGTGTCCACGCCAGCGCTCCTAGTGGCCATGCACGTGGCCTACCGG
AGACATGAGAAGAAGAGGAAGTTCATCAAGGGGGAGATAAAGAGTGAATTTAAGGACATCGA
GGAGATCAAAACCCAGAAGGTCCGCATCGAAGGCTCCCTGTGGTGGACCTACACAAGCAGCA
TCTTCTTCCGGGTCATCTTCGAAGCCGCCTTCATGTACGTCTTCTATGTCATGTACGACGGC
TTCTCCATGCAGCGGCTGGTGAAGTGCAACGCCTGGCCTTGTCCCAACACTGTGGACTGCTT
TGTGTCCCGGCCCACGGAGAAGACTGTCTTCACAGTGTTCATGATTGCAGTGTCTGGAATTT
GCATCCTGCTGAATGTCACTGAATTGTGTTATTTGCTAATTAGATATTGTTCTGGGAAGTCA
AAAAAGCCAGTTTAAAGGCGCGCCACCCCTGCAGGGAATTCCGCATTGCCCAGTTGTTAGAT
TAAGAAATAGACAGCATGAGAGGGATGAGGCAACCCGTGCTCAGCTGTCAAGGCTCAGTCGC
TAGCATTTCCCAACACAAAGATTCTGACCTTAAATGCAACCATTTGAAACCCCTGTAGGCCT
CAGGTGAAACTCCAGATGCCACAATGGAGCTCTGCTCCCCTAAAGCCTCAAAACAAAGGCCT
AATTCTATGCCTGTCTTAATTTTCTTTCACTTAAGTTAGTTCCACTGAGACCCCAGGCTGTT
AGGGGTTATTGGTGTAAGGTACTTTCATATTTTAAACAGAGGATATCGGCATTTGTTTCTTT
CTCTGAGGACAAGAGAAAAAAGCCAGGTTCCACAGAGGACACAGAGAAGGTTTGGGTGTCCT
CCTGGGGTTCTTTTTGCCAACTTTCCCCACGTTAAAGGTGAACATTGGTTCTTTCATTTGCT
TTGGAAGTTTTAATCTCTAACAGTGGACAAAGTTACCAGTGCCTTAAACTCTGTTACACTTT
TTGGAAGTGAAAACTTTGTAGTATGATAGGTTATTTTGATGTAAAGATGTTCTGGATACCAT
TATATGTTCCCCCTGTTTCAGAGGCTCAGATTGTAATATGTAAATGGTATGTCATTCGCTAC
TATGATTTAATTTGAAATATGGTCTTTTGGTTATGAATACTTTGCAGCACAGCTGAGAGGCT
GTCTGTTGTATTCATTGTGGTCATAGCACCTAACAACATTGTAGCCTCAATCGAGTGAGACA
GACTAGAAGTTCCTAGTGATGGCTTATGATAGCAAATGGCCTCATGTCAAATATTTAGATGT
AATTTTGTGTAAGAAATACAGACTGGATGTACCACCAACTACTACCTGTAATGACAGGCCTG
TCCAACACATCTCCCTTTTCCATGACTGTGGTAGCCAGCATCGGAAAGAACGCTGATTTAAA
GAGGTCGCTTGGGAATTTTATTGACACAGTACCATTTAATGGGGAGGACAAAATGGGGCAGG
GGAGGGAGAAGTTTCTGTCGTTAAAAACAGATTTGGAAAGACTGGACTCTAAAGTCTGTTGA
TTAAAGATGAGCTTTGTCTACTTCAAAAGTTTGTTTGCTTACCCCTTCAGCCTCCAATTTTT
TAAGTGAAAATATAGCTAATAACATGTGAAAAGAATAGAAGCTAAGGTTTAGATAAATATTG
AGCAGATCTATAGGAAGATTGAACCTGAATATTGCCATTATGCTTGACATGGTTTCCAAAAA
ATGGTACTCCACATATTTCAGTGAGGGTAAGTATTTTCCTGTTGTCAAGAATAGCATTGTAA
AAGCATTTTGTAATAATAAAGAATAGCTTTAATGATATGCTTGTAACTAAAATAATTTTGTA
ATGTATCAAATACATTTAAAACATTAAAATATAATCTCTATAATAATTTAAAATCTAATATG
GTTTTAATAGAACAGCGATATCAAGCTTATCGATAATCAACCTCTGGATTACAAAATTTGTG
AAAGATTGACTGGTATTCTTAACTATGTTGCTCCTTTTACGCTATGTGGATACGCTGCTTTA
ATGCCTTTGTATCATGCTATTGCTTCCCGTATGGCTTTCATTTTCTCCTCCTTGTATAAATC
CTGGTTGCTGTCTCTTTATGAGGAGTTGTGGCCCGTTGTCAGGCAACGTGGCGTGGTGTGCA
CTGTGTTTGCTGACGCAACCCCCACTGGTTGGGGCATTGCCACCACCTGTCAGCTCCTTTCC
GGGACTTTCGCTTTCCCCCTCCCTATTGCCACGGCGGAACTCATCGCCGCCTGCCTTGCCCG
CTGCTGGACAGGGGCTCGGCTGTTGGGCACTGACAATTCCGTGGTGTTGTCGGGGAAATCAT
CGTCCTTTCCTTGGCTGCTCGCCTATGTTGCCACCTGGATTCTGCGCGGGACGTCCTTCTGC
TACGTCCCTTCGGCCCTCAATCCAGCGGACCTTCCTTCCCGCGGCCTGCTGCCGGCTCTGCG
GCCTCTTCCGCGTCTTCGCCTTCGCCCTCAGACGAGTCGGATCTCCCTTTGGGCCGCCTCCC
CGCGAATTCATCGATACCGAGCGCTGCTCGAGAGATCTGTGATAGCGGCCATCAAGCTGGCT
GTGCCTTCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGTCTGAGTA
GGTGTCATTCTATTCTGGGGGGTGGGGTGGGGCAGGACAGCAAGGGGGAGGATTGGGAAGAC
AATAGCAGGCATGCTGGGGACACGTGCGGACCGAGCGGCCGCAGGAACCCCTAGTGATGGAG
TTGGCCACTCCCTCTCTGCGCGCTCGCTCGCTCACTGAGGCCGGGCGACCAAAGGTCGCCCG
ACGCCCGGGCTTTGCCCGGGCGGCCTCAGTGAGCGAGCGAGCGCGCAGCTGCCTGCAGGGGC
GCCTGATGCGGTATTTTCTCCTTACGCATCTGTGCGGTATTTCACACCGCATACGTCAAAGC
AACCATAGTACGCGCCCTGTAGCGGCGCATTAAGCGCGGCGGGTGTGGTGGTTACGCGCAGC
GTGACCGCTACACTTGCCAGCGCCCTAGCGCCCGCTCCTTTCGCTTTCTTCCCTTCCTTTCT
CGCCACGTTCGCCGGCTTTCCCCGTCAAGCTCTAAATCGGGGGCTCCCTTTAGGGTTCCGAT
TTAGTGCTTTACGGCACCTCGACCCCAAAAAACTTGATTTGGGTGATGGTTCACGTAGTGGG
CCATCGCCCTGATAGACGGTTTTTCGCCCTTTGACGTTGGAGTCCACGTTCTTTAATAGTGG
ACTCTTGTTCCAAACTGGAACAACACTCAACCCTATCTCGGGCTATTCTTTTGATTTATAAG
GGATTTTGCCGATTTCGGCCTATTGGTTAAAAAATGAGCTGATTTAACAAAAATTTAACGCG
AATTTTAACAAAATATTAACGTTTACAATTTTATGGTGCACTCTCAGTACAATCTGCTCTGA
TGCCGCATAGTTAAGCCAGCCCCGACACCCGCCAACACCCGCTGACGCGCCCTGACGGGCTT
GTCTGCTCCCGGCATCCGCTTACAGACAAGCTGTGACCGTCTCCGGGAGCTGCATGTGTCAG
AGGTTTTCACCGTCATCACCGAAACGCGCGAGACGAAAGGGCCTCGTGATACGCCTATTTTT
ATAGGTTAATGTCATGATAATAATGGTTTCTTAGACGTCAGGTGGCACTTTTCGGGGAAATG
TGCGCGGAACCCCTATTTGTTTATTTTTCTAAATACATTCAAATATGTATCCGCTCATGAGA
CAATAACCCTGATAAATGCTTCAATAATATTGAAAAAGGAAGAGTATGAGTATTCAACATTT
CCGTGTCGCCCTTATTCCCTTTTTTGCGGCATTTTGCCTTCCTGTTTTTGCTCACCCAGAAA
CGCTGGTGAAAGTAAAAGATGCTGAAGATCAGTTGGGTGCACGAGTGGGTTACATCGAACTG
GATCTCAACAGCGGTAAGATCCTTGAGAGTTTTCGCCCCGAAGAACGTTTTCCAATGATGAG
CACTTTTAAAGTTCTGCTATGTGGCGCGGTATTATCCCGTATTGACGCCGGGCAAGAGCAAC
TCGGTCGCCGCATACACTATTCTCAGAATGACTTGGTTGAGTACTCACCAGTCACAGAAAAG
CATCTTACGGATGGCATGACAGTAAGAGAATTATGCAGTGCTGCCATAACCATGAGTGATAA
CACTGCGGCCAACTTACTTCTGACAACGATCGGAGGACCGAAGGAGCTAACCGCTTTTTTGC
ACAACATGGGGGATCATGTAACTCGCCTTGATCGTTGGGAACCGGAGCTGAATGAAGCCATA
CCAAACGACGAGCGTGACACCA

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 78. An exemplary nucleotide sequence for vector c.81.7 is set forth in SEQ ID NO: 78 (hGJB2 GRE7 underlined; mouse GJB2 coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTC
TCAGCTGGAGTGACGCACCTCATCCATGCGGGCCTGGCGTCTGGAAGGTGGCTGGGTCTCTC
GGGCTTGAGCACCATCATCTTAGCTCCAACATGTCATTATTCCTTCCTCACTGAGGACTTTT
CTGCTTCCTAATTGGTTGTTGAAGATGAGGCCCCCATGCTCTTTTAAGAAAACCTGTTGTGC
CCCAGGCTTGGCTGTGATGGGCACTGACTCATACAGAAGTAGAAAGGCCTGCTGAGTCATCA
ACACTCGTGCGACGCCCTCGCATTTTCATTAATGATGGCCTCCCTGCCACACGTGAATCACT
CCAGCCCGAGATCTGAAACCAGGACACACCCCAGGGGCGAGGTGACGCTGAGTGAGCCCAGC
TGTGTCCCTTTCATGAGAACTCAGAGCACAGGGCTCTGTGTGCATGGCCGTCCCCTCCAGAG
AGGAGGAAGTAAATGCCGGGATTAGTGGAAGATCATTTCCTTCTATTTGCCTTGGCTTACGT
CTTTCAGAATTCAAACACGTGCACTGTTGACCCTGCAATGGTGGAGTTTTTGGATTTTCCTT
CAGTCCGATTGCTAAAATACTTCCCTCTCATGTGAGCTGTTGTGAAAGTCATCAGCCAGATA
CCATTCTAAAAACAAAGAATGTGCTTCTCGTATGTTGCATGCTGGTTACTGAAATATTAGGG
AATTACATAAAGGTTTTCTGGGGCACATATTCAAGCTGAATGATAAAATTGAAGGTCACACA
AAGCTAAGGTCTTTCAAATCCTGACCCAATTAGCTCTCTGTTAGCTCTCTGACTTTGGACAA
GCTGTCTGGTCCTCTGAAGCATACTTTGTTCGCCCTGGGTAGGGGCCCTCTGTTTTAACAGC
GTTTGGCATTAATTAAGACCTCGAAGGGGACTTGGGGGGTTCGGGGCTTTCGGGGGCGGTCG
GGGGTTCGCGGACCCGGGAAGCTCTGAGGACCCAGAGGCCGGGCGCGCTCCGCCCGCGGCGC
CGCCCCCTCCGTAACTTTCCCAGTCTCCGAGGGAAGAGGCGGGGTGTGGGGTGCGGTTAAAA
GGCGCCACGGCGGGAGACAGGTGTTGCGGCCCCGCAGCGCCCGCGCGCTCCTCTCCCCGACT
CGGAGCCCCTCGGCGGCGCCCGGCCCAGGACCCGCCTAGGAGCGCAGGAGCCCCAGCGCAGA
GACCCCAACGCCGAGACCCCCGCCCCGGCCCCGCCGCGCTTCCTCCCGACGCAGAGCAAACC
GCCCAGAGTAGAAGCCATGGATTGGGGCACACTCCAGAGCATCCTCGGGGGTGTCAACAAAC
ACTCCACCAGCATTGGAAAGATCTGGCTCACGGTCCTCTTCATCTTCCGCATCATGATCCTC
GTGGTGGCTGCAAAGGAGGTGTGGGGAGATGAGCAAGCCGATTTTGTCTGCAACACGCTCCA
GCCTGGCTGCAAGAATGTATGCTACGACCACCACTTCCCCATCTCTCACATCCGGCTCTGGG
CTCTGCAGCTGATCATGGTGTCCACGCCAGCCCTCCTGGTAGCTATGCATGTGGCCTACCGG
AGACATGAAAAGAAACGGAAGTTCATGAAGGGAGAGATAAAGAACGAGTTTAAGGACATCGA
AGAGATCAAAACCCAGAAGGTCCGTATCGAAGGGTCCCTGTGGTGGACCTACACCACCAGCA
TCTTCTTCCGGGTCATCTTTGAAGCCGTCTTCATGTACGTCTTTTACATCATGTACAATGGC
TTCTTCATGCAACGTCTGGTGAAATGCAACGCTTGGCCCTGCCCCAATACAGTGGACTGCTT
CATTTCCAGGCCCACAGAAAAGACTGTCTTCACCGTGTTTATGATTTCTGTGTCTGGAATTT
GCATTCTGCTAAATATCACAGAGCTGTGCTATTTGTTCGTTAGGTATTGCTCAGGAAAGTCC
AAAAGACCAGTCTACCCATACGATGTTCCAGATTACGCTTAAAGGCGCGCCACCCCTGCAGG
GAATTCCGCATTGCCCAGTTGTTAGATTAAGAAATAGACAGCATGAGAGGGATGAGGCAACC
CGTGCTCAGCTGTCAAGGCTCAGTCGCTAGCATTTCCCAACACAAAGATTCTGACCTTAAAT
GCAACCATTTGAAACCCCTGTAGGCCTCAGGTGAAACTCCAGATGCCACAATGGAGCTCTGC
TCCCCTAAAGCCTCAAAACAAAGGCCTAATTCTATGCCTGTCTTAATTTTCTTTCACTTAAG
TTAGTTCCACTGAGACCCCAGGCTGTTAGGGGTTATTGGTGTAAGGTACTTTCATATTTTAA
ACAGAGGATATCGGCATTTGTTTCTTTCTCTGAGGACAAGAGAAAAAAGCCAGGTTCCACAG
AGGACACAGAGAAGGTTTGGGTGTCCTCCTGGGGTTCTTTTTGCCAACTTTCCCCACGTTAA
AGGTGAACATTGGTTCTTTCATTTGCTTTGGAAGTTTTAATCTCTAACAGTGGACAAAGTTA
CCAGTGCCTTAAACTCTGTTACACTTTTTGGAAGTGAAAACTTTGTAGTATGATAGGTTATT
TTGATGTAAAGATGTTCTGGATACCATTATATGTTCCCCCTGTTTCAGAGGCTCAGATTGTA
ATATGTAAATGGTATGTCATTCGCTACTATGATTTAATTTGAAATATGGTCTTTTGGTTATG
AATACTTTGCAGCACAGCTGAGAGGCTGTCTGTTGTATTCATTGTGGTCATAGCACCTAACA
ACATTGTAGCCTCAATCGAGTGAGACAGACTAGAAGTTCCTAGTGATGGCTTATGATAGCAA
ATGGCCTCATGTCAAATATTTAGATGTAATTTTGTGTAAGAAATACAGACTGGATGTACCAC
CAACTACTACCTGTAATGACAGGCCTGTCCAACACATCTCCCTTTTCCATGACTGTGGTAGC
CAGCATCGGAAAGAACGCTGATTTAAAGAGGTCGCTTGGGAATTTTATTGACACAGTACCAT
TTAATGGGGAGGACAAAATGGGGCAGGGGAGGGAGAAGTTTCTGTCGTTAAAAACAGATTTG
GAAAGACTGGACTCTAAAGTCTGTTGATTAAAGATGAGCTTTGTCTACTTCAAAAGTTTGTT
TGCTTACCCCTTCAGCCTCCAATTTTTTAAGTGAAAATATAGCTAATAACATGTGAAAAGAA
TAGAAGCTAAGGTTTAGATAAATATTGAGCAGATCTATAGGAAGATTGAACCTGAATATTGC
CATTATGCTTGACATGGTTTCCAAAAAATGGTACTCCACATATTTCAGTGAGGGTAAGTATT
TTCCTGTTGTCAAGAATAGCATTGTAAAAGCATTTTGTAATAATAAAGAATAGCTTTAATGA
TATGCTTGTAACTAAAATAATTTTGTAATGTATCAAATACATTTAAAACATTAAAATATAAT
CTCTATAATAATTTAAAATCTAATATGGTTTTAATAGAACAGCGATATCAAGCTTATCGATA
ATCAACCTCTGGATTACAAAATTTGTGAAAGATTGACTGGTATTCTTAACTATGTTGCTCCT
TTTACGCTATGTGGATACGCTGCTTTAATGCCTTTGTATCATGCTATTGCTTCCCGTATGGC
TTTCATTTTCTCCTCCTTGTATAAATCCTGGTTGCTGTCTCTTTATGAGGAGTTGTGGCCCG
TTGTCAGGCAACGTGGCGTGGTGTGCACTGTGTTTGCTGACGCAACCCCCACTGGTTGGGGC
ATTGCCACCACCTGTCAGCTCCTTTCCGGGACTTTCGCTTTCCCCCTCCCTATTGCCACGGC
GGAACTCATCGCCGCCTGCCTTGCCCGCTGCTGGACAGGGGCTCGGCTGTTGGGCACTGACA
ATTCCGTGGTGTTGTCGGGGAAATCATCGTCCTTTCCTTGGCTGCTCGCCTATGTTGCCACC
TGGATTCTGCGCGGGACGTCCTTCTGCTACGTCCCTTCGGCCCTCAATCCAGCGGACCTTCC
TTCCCGCGGCCTGCTGCCGGCTCTGCGGCCTCTTCCGCGTCTTCGCCTTCGCCCTCAGACGA
GTCGGATCTCCCTTTGGGCCGCCTCCCCGCGAATTCATCGATACCGAGCGCTGCTCGAGAGA
TCTGTGATAGCGGCCATCAAGCTGGCTGTGCCTTCTAGTTGCCAGCCATCTGTTGTTTGCCC
CTCCCCCGTGCCTTCCTTGACCCTGGAAGGTGCCACTCCCACTGTCCTTTCCTAATAAAATG
AGGAAATTGCATCGCATTGTCTGAGTAGGTGTCATTCTATTCTGGGGGGTGGGGTGGGGCAG
GACAGCAAGGGGGAGGATTGGGAAGACAATAGCAGGCATGCTGGGGACACGTGCGGACCGAG
CGGCCGCAGGAACCCCTAGTGATGGAGTTGGCCACTCCCTCTCTGCGCGCTCGCTCGCTCAC
TGAGGCCGGGCGACCAAAGGTCGCCCGACGCCCGGGCTTTGCCCGGGCGGCCTCAGTGAGCG
AGCGAGCGCGCAGCTGCCTGCAGGGGCGCCTGATGCGGTATTTTCTCCTTACGCATCTGTGC
GGTATTTCACACCGCATACGTCAAAGCAACCATAGTACGCGCCCTGTAGCGGCGCATTAAGC
GCGGCGGGTGTGGTGGTTACGCGCAGCGTGACCGCTACACTTGCCAGCGCCCTAGCGCCCGC
TCCTTTCGCTTTCTTCCCTTCCTTTCTCGCCACGTTCGCCGGCTTTCCCCGTCAAGCTCTAA
ATCGGGGGCTCCCTTTAGGGTTCCGATTTAGTGCTTTACGGCACCTCGACCCCAAAAAACTT
GATTTGGGTGATGGTTCACGTAGTGGGCCATCGCCCTGATAGACGGTTTTTCGCCCTTTGAC
GTTGGAGTCCACGTTCTTTAATAGTGGACTCTTGTTCCAAACTGGAACAACACTCAACCCTA
TCTCGGGCTATTCTTTTGATTTATAAGGGATTTTGCCGATTTCGGCCTATTGGTTAAAAAAT
GAGCTGATTTAACAAAAATTTAACGCGAATTTTAACAAAATATTAACGTTTACAATTTTATG
GTGCACTCTCAGTACAATCTGCTCTGATGCCGCATAGTTAAGCCAGCCCCGACACCCGCCAA
CACCCGCTGACGCGCCCTGACGGGCTTGTCTGCTCCCGGCATCCGCTTACAGACAAGCTGTG
ACCGTCTCCGGGAGCTGCATGTGTCAGAGGTTTTCACCGTCATCACCGAAACGCGCGAGACG
AAAGGGCCTCGTGATACGCCTATTTTTATAGGTTAATGTCATGATAATAATGGTTTCTTAGA
CGTCAGGTGGCACTTTTCGGGGAAATGTGCGCGGAACCCCTATTTGTTTATTTTTCTAAATA
CATTCAAATATGTATCCGCTCATGAGACAATAACCCTGATAAATGCTTCAATAATATTGAAA
AAGGAAGAGTATGAGTATTCAACATTTCCGTGTCGCCCTTATTCCCTTTTTTGCGGCATTTT
GCCTTCCTGTTTTTGCTCACCCAGAAACGCTGGTGAAAGTAAAAGATGCTGAAGATCAGTTG
GGTGCACGAGTGGGTTACATCGAACTGGATCTCAACAGCGGTAAGATCCTTGAGAGTTTTCG
CCCCGAAGAACGTTTTCCAATGATGAGCACTTTTAAAGTTCTGCTATGTGGCGCGGTATTAT
CCCGTATTGACGCCGGGCAAGAGCAACTCGGTCGCCGCATACACTATTCTCAGAATGACTTG
GTTGAGTACTCACCAGTCACAGAAAAGCATCTTACGGATGGCATGACAGTAAGAGAATTATG
CAGTGCTGCCATAACCATGAGTGATAACACTGCGGCCAACTTACTTCTGACAACGATCGGAG
GACCGAAGGAGCTAACCGCTTTTTTGCACAACATGGGGGATCATGTAACTCGCCTTGATCGT
TGGGAACCGGAGCTGAATGAAGCCATACCAAACGACGAGCGTGACACCA

In some embodiments, an AAV vector described herein comprises an AAV 5′ ITR, a GJB2 GRE enhancer (hGJB2 GRE8), a GJB2 basal promoter, a GJB2 exon 1 5′ UTR, Kozak sequence, nucleotide sequence encoding a gene product (e.g., GJB2 or GFP), a GJB2 exon 2 3′ UTR, a WPRE, a bovine growth hormone poly A signal, and an AAV 3′ ITR (e.g., vector c.81.8).

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 79. An exemplary nucleotide sequence for vector c.81.8 is set forth in SEQ ID NO: 79 (hGJB2 GRE8 underlined; eGFP coding sequence in bold face):

CGATGCCTGTAGCAATGGCAACAACGTTGCGCAAACTATTAACTGGCGAACTACTT
ACTCTAGCTTCCCGGCAACAATTAATAGACTGGATGGAGGCGGATAAAGTTGCAGGACCACT
TCTGCGCTCGGCCCTTCCGGCTGGCTGGTTTATTGCTGATAAATCTGGAGCCGGTGAGCGTG
GGTCTCGCGGTATCATTGCAGCACTGGGGCCAGATGGTAAGCCCTCCCGTATCGTAGTTATC
TACACGACGGGGAGTCAGGCAACTATGGATGAACGAAATAGACAGATCGCTGAGATAGGTGC
CTCACTGATTAAGCATTGGTAACTGTCAGACCAAGTTTACTCATATATACTTTAGATTGATT
TAAAACTTCATTTTTAATTTAAAAGGATCTAGGTGAAGATCCTTTTTGATAATCTCATGACC
AAAATCCCTTAACGTGAGTTTTCGTTCCACTGAGCGTCAGACCCCGTAGAAAAGATCAAAGG
ATCTTCTTGAGATCCTTTTTTTCTGCGCGTAATCTGCTGCTTGCAAACAAAAAAACCACCGC
TACCAGCGGTGGTTTGTTTGCCGGATCAAGAGCTACCAACTCTTTTTCCGAAGGTAACTGGC
TTCAGCAGAGCGCAGATACCAAATACTGTCCTTCTAGTGTAGCCGTAGTTAGGCCACCACTT
CAAGAACTCTGTAGCACCGCCTACATACCTCGCTCTGCTAATCCTGTTACCAGTGGCTGCTG
CCAGTGGCGATAAGTCGTGTCTTACCGGGTTGGACTCAAGACGATAGTTACCGGATAAGGCG
CAGCGGTCGGGCTGAACGGGGGGTTCGTGCACACAGCCCAGCTTGGAGCGAACGACCTACAC
CGAACTGAGATACCTACAGCGTGAGCTATGAGAAAGCGCCACGCTTCCCGAAGGGAGAAAGG
CGGACAGGTATCCGGTAAGCGGCAGGGTCGGAACAGGAGAGCGCACGAGGGAGCTTCCAGGG
GGAAACGCCTGGTATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACGCGGCCTTTTTAC
GGTTCCTGGCCTTTTGCTGGCCTTTTGCTCACATGTCCTGCAGGCAGCTGCGCGCTCGCTCG
CTCACTGAGGCCGCCCGGGCAAAGCCCGGGCGTCGGGCGACCTTTGGTCGCCCGGCCTCAGT
GAGCGAGCGAGCGCGCAGAGAGGGAGTGGCCAACTCCATCACTAGGGGTTCCTGCGGCCGCA
CGCGTCGACCTGAACGATTAAGGCAAAACTTCGAAATGTGCCCCAGCAGAGATTTATTTTTC
AGGGGGTGTTTTGCATTCCAGCCCCTCTGCCTTCCTGGCGTTTAGTGCGATTTGTTTAGCCA
TGTGCTCCCTGGTGTGTGTTTTTGAATGTGTGTGAGATGGGTTGTCTCTCGGGACCTGGCAG
GTGCGGCCACCAGGTCAGGGCTGCCCCCCAACCCTGTGCCTCCTTCCTCCTAGACTCTGGCC
CCCTCAGTGCTGAGGGTGATACAGAGCACTTTTCAAGCTGGATTTGGAATGTGGCCTCTCCC
CTCCAAACTCCTGGAGATCATGCAAAGGCCTTTGGAGCCAGCCAGTCACCTGGAAGGTGACA
TTCCCACCAGCTGAGGCCTCACCTTCAGCGGGGGCTGGGCAGCTTTGGAGCCTGGGGCCAGC
CAAGCTCACTCTGCCCATATCCCTGCCACGTGTGGCCCAGCGGATGATCACCTGTCTTCATC
TGCGTACTGGGCCACATCCCTCCTGCCGTCCCCCACTTCCCTGATGACACCTACAGCAAGCC
CCTACCCAAGTGTTCTGTGATCCCCTGTAAATGTGGCCTCCCTAGCTACTTGCTTTTATGAA
ACCAACAATCCTGGGGACACAGTTTTCGGCTGTCTCAAGACGGGGCAACCACTCTTTTCCCC
AGGCCTGTGGGTCCCAGGCCTGGAGCTAGGGTTGGCATTCTTGCCTGAATTCTCCACTCTAT
CCCAACCCCTGAGGCCGCCTGAGGAGGCTCAGACTGTGTCAGGCTAGGAGGACAGTCAAACC
ACAAAAACATGCCTTTTAAGAAGTATAAGCACAAATCCCTCTTTGATGTTATATAAAAGCTC
AGTGTCACTTTAATTAAGACCTCGAAGGGGACTTGGGGGGTTCGGGGCTTTCGGGGGCGGTC
GGGGGTTCGCGGACCCGGGAAGCTCTGAGGACCCAGAGGCCGGGCGCGCTCCGCCCGCGGCG
CCGCCCCCTCCGTAACTTTCCCAGTCTCCGAGGGAAGAGGCGGGGTGTGGGGTGCGGTTAAA
AGGCGCCACGGCGGGAGACAGGTGTTGCGGCCCCGCAGCGCCCGCGCGCTCCTCTCCCCGAC
TCGGAGCCCCTCGGCGGCGCCCGGCCCAGGACCCGCCTAGGAGCGCAGGAGCCCCAGCGCAG
AGACCCCAACGCCGAGACCCCCGCCCCGGCCCCGCCGCGCTTCCTCCCGACGCAGAGCAAAC
CGCCCAGAGTAGAAGCCATGGTGAGCAAGGGCGAGGAGCTGTTCACCGGGGTGGTGCCCATC
CTGGTCGAGCTGGACGGCGACGTAAACGGCCACAAGTTCAGCGTGTCCGGCGAGGGCGAGGG
CGATGCCACCTACGGCAAGCTGACCCTGAAGTTCATCTGCACCACCGGCAAGCTGCCCGTGC
CCTGGCCCACCCTCGTGACCACCCTGACCTACGGCGTGCAGTGCTTCAGCCGCTACCCCGAC
CACATGAAGCAGCACGACTTCTTCAAGTCCGCCATGCCCGAAGGCTACGTCCAGGAGCGCAC
CATCTTCTTCAAGGACGACGGCAACTACAAGACCCGCGCCGAGGTGAAGTTCGAGGGCGACA
CCCTGGTGAACCGCATCGAGCTGAAGGGCATCGACTTCAAGGAGGACGGCAACATCCTGGGG
CACAAGCTGGAGTACAACTACAACAGCCACAACGTCTATATCATGGCCGACAAGCAGAAGAA
CGGCATCAAGGTGAACTTCAAGATCCGCCACAACATCGAGGACGGCAGCGTGCAGCTCGCCG
ACCACTACCAGCAGAACACCCCCATCGGCGACGGCCCCGTGCTGCTGCCCGACAACCACTAC
CTGAGCACCCAGTCCGCCCTGAGCAAAGACCCCAACGAGAAGCGCGATCACATGGTCCTGCT
GGAGTTCGTGACCGCCGCCGGGATCACTCTCGGCATGGACGAGCTGTACAAGTAATAAAGGC
GCGCCACCCCTGCAGGGAATTCCGCATTGCCCAGTTGTTAGATTAAGAAATAGACAGCATGA
GAGGGATGAGGCAACCCGTGCTCAGCTGTCAAGGCTCAGTCGCTAGCATTTCCCAACACAAA
GATTCTGACCTTAAATGCAACCATTTGAAACCCCTGTAGGCCTCAGGTGAAACTCCAGATGC
CACAATGGAGCTCTGCTCCCCTAAAGCCTCAAAACAAAGGCCTAATTCTATGCCTGTCTTAA
TTTTCTTTCACTTAAGTTAGTTCCACTGAGACCCCAGGCTGTTAGGGGTTATTGGTGTAAGG
TACTTTCATATTTTAAACAGAGGATATCGGCATTTGTTTCTTTCTCTGAGGACAAGAGAAAA
AAGCCAGGTTCCACAGAGGACACAGAGAAGGTTTGGGTGTCCTCCTGGGGTTCTTTTTGCCA
ACTTTCCCCACGTTAAAGGTGAACATTGGTTCTTTCATTTGCTTTGGAAGTTTTAATCTCTA
ACAGTGGACAAAGTTACCAGTGCCTTAAACTCTGTTACACTTTTTGGAAGTGAAAACTTTGT
AGTATGATAGGTTATTTTGATGTAAAGATGTTCTGGATACCATTATATGTTCCCCCTGTTTC
AGAGGCTCAGATTGTAATATGTAAATGGTATGTCATTCGCTACTATGATTTAATTTGAAATA
TGGTCTTTTGGTTATGAATACTTTGCAGCACAGCTGAGAGGCTGTCTGTTGTATTCATTGTG
GTCATAGCACCTAACAACATTGTAGCCTCAATCGAGTGAGACAGACTAGAAGTTCCTAGTGA
TGGCTTATGATAGCAAATGGCCTCATGTCAAATATTTAGATGTAATTTTGTGTAAGAAATAC
AGACTGGATGTACCACCAACTACTACCTGTAATGACAGGCCTGTCCAACACATCTCCCTTTT
CCATGACTGTGGTAGCCAGCATCGGAAAGAACGCTGATTTAAAGAGGTCGCTTGGGAATTTT
ATTGACACAGTACCATTTAATGGGGAGGACAAAATGGGGCAGGGGAGGGAGAAGTTTCTGTC
GTTAAAAACAGATTTGGAAAGACTGGACTCTAAAGTCTGTTGATTAAAGATGAGCTTTGTCT
ACTTCAAAAGTTTGTTTGCTTACCCCTTCAGCCTCCAATTTTTTAAGTGAAAATATAGCTAA
TAACATGTGAAAAGAATAGAAGCTAAGGTTTAGATAAATATTGAGCAGATCTATAGGAAGAT
TGAACCTGAATATTGCCATTATGCTTGACATGGTTTCCAAAAAATGGTACTCCACATATTTC
AGTGAGGGTAAGTATTTTCCTGTTGTCAAGAATAGCATTGTAAAAGCATTTTGTAATAATAA
AGAATAGCTTTAATGATATGCTTGTAACTAAAATAATTTTGTAATGTATCAAATACATTTAA
AACATTAAAATATAATCTCTATAATAATTTAAAATCTAATATGGTTTTAATAGAACAGCGAT
ATCAAGCTTATCGATAATCAACCTCTGGATTACAAAATTTGTGAAAGATTGACTGGTATTCT
TAACTATGTTGCTCCTTTTACGCTATGTGGATACGCTGCTTTAATGCCTTTGTATCATGCTA
TTGCTTCCCGTATGGCTTTCATTTTCTCCTCCTTGTATAAATCCTGGTTGCTGTCTCTTTAT
GAGGAGTTGTGGCCCGTTGTCAGGCAACGTGGCGTGGTGTGCACTGTGTTTGCTGACGCAAC
CCCCACTGGTTGGGGCATTGCCACCACCTGTCAGCTCCTTTCCGGGACTTTCGCTTTCCCCC
TCCCTATTGCCACGGCGGAACTCATCGCCGCCTGCCTTGCCCGCTGCTGGACAGGGGCTCGG
CTGTTGGGCACTGACAATTCCGTGGTGTTGTCGGGGAAATCATCGTCCTTTCCTTGGCTGCT
CGCCTATGTTGCCACCTGGATTCTGCGCGGGACGTCCTTCTGCTACGTCCCTTCGGCCCTCA
ATCCAGCGGACCTTCCTTCCCGCGGCCTGCTGCCGGCTCTGCGGCCTCTTCCGCGTCTTCGC
CTTCGCCCTCAGACGAGTCGGATCTCCCTTTGGGCCGCCTCCCCGCGAATTCATCGATACCG
AGCGCTGCTCGAGAGATCTGTGATAGCGGCCATCAAGCTGGCTGTGCCTTCTAGTTGCCAGC
CATCTGTTGTTTGCCCCTCCCCCGTGCCTTCCTTGACCCTGGAAGGTGCCACTCCCACTGTC
CTTTCCTAATAAAATGAGGAAATTGCATCGCATTGTCTGAGTAGGTGTCATTCTATTCTGGG
GGGTGGGGTGGGGCAGGACAGCAAGGGGGAGGATTGGGAAGACAATAGCAGGCATGCTGGGG
ACACGTGCGGACCGAGCGGCCGCAGGAACCCCTAGTGATGGAGTTGGCCACTCCCTCTCTGC
GCGCTCGCTCGCTCACTGAGGCCGGGCGACCAAAGGTCGCCCGACGCCCGGGCTTTGCCCGG
GCGGCCTCAGTGAGCGAGCGAGCGCGCAGCTGCCTGCAGGGGCGCCTGATGCGGTATTTTCT
CCTTACGCATCTGTGCGGTATTTCACACCGCATACGTCAAAGCAACCATAGTACGCGCCCTG
TAGCGGCGCATTAAGCGCGGCGGGTGTGGTGGTTACGCGCAGCGTGACCGCTACACTTGCCA
GCGCCCTAGCGCCCGCTCCTTTCGCTTTCTTCCCTTCCTTTCTCGCCACGTTCGCCGGCTTT
CCCCGTCAAGCTCTAAATCGGGGGCTCCCTTTAGGGTTCCGATTTAGTGCTTTACGGCACCT
CGACCCCAAAAAACTTGATTTGGGTGATGGTTCACGTAGTGGGCCATCGCCCTGATAGACGG
TTTTTCGCCCTTTGACGTTGGAGTCCACGTTCTTTAATAGTGGACTCTTGTTCCAAACTGGA
ACAACACTCAACCCTATCTCGGGCTATTCTTTTGATTTATAAGGGATTTTGCCGATTTCGGC
CTATTGGTTAAAAAATGAGCTGATTTAACAAAAATTTAACGCGAATTTTAACAAAATATTAA
CGTTTACAATTTTATGGTGCACTCTCAGTACAATCTGCTCTGATGCCGCATAGTTAAGCCAG
CCCCGACACCCGCCAACACCCGCTGACGCGCCCTGACGGGCTTGTCTGCTCCCGGCATCCGC
TTACAGACAAGCTGTGACCGTCTCCGGGAGCTGCATGTGTCAGAGGTTTTCACCGTCATCAC
CGAAACGCGCGAGACGAAAGGGCCTCGTGATACGCCTATTTTTATAGGTTAATGTCATGATA
ATAATGGTTTCTTAGACGTCAGGTGGCACTTTTCGGGGAAATGTGCGCGGAACCCCTATTTG
TTTATTTTTCTAAATACATTCAAATATGTATCCGCTCATGAGACAATAACCCTGATAAATGC
TTCAATAATATTGAAAAAGGAAGAGTATGAGTATTCAACATTTCCGTGTCGCCCTTATTCCC
TTTTTTGCGGCATTTTGCCTTCCTGTTTTTGCTCACCCAGAAACGCTGGTGAAAGTAAAAGA
TGCTGAAGATCAGTTGGGTGCACGAGTGGGTTACATCGAACTGGATCTCAACAGCGGTAAGA
TCCTTGAGAGTTTTCGCCCCGAAGAACGTTTTCCAATGATGAGCACTTTTAAAGTTCTGCTA
TGTGGCGCGGTATTATCCCGTATTGACGCCGGGCAAGAGCAACTCGGTCGCCGCATACACTA
TTCTCAGAATGACTTGGTTGAGTACTCACCAGTCACAGAAAAGCATCTTACGGATGGCATGA
CAGTAAGAGAATTATGCAGTGCTGCCATAACCATGAGTGATAACACTGCGGCCAACTTACTT
CTGACAACGATCGGAGGACCGAAGGAGCTAACCGCTTTTTTGCACAACATGGGGGATCATGT
AACTCGCCTTGATCGTTGGGAACCGGAGCTGAATGAAGCCATACCAAACGACGAGCGTGACA
CCA

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 80. An exemplary nucleotide sequence for vector c.81.8 is set forth in SEQ ID NO: 80 (hGJB2 GRE8 underlined; human GJB2 coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTC
GACCTGAACGATTAAGGCAAAACTTCGAAATGTGCCCCAGCAGAGATTTATTTTTCAGGGGG
TGTTTTGCATTCCAGCCCCTCTGCCTTCCTGGCGTTTAGTGCGATTTGTTTAGCCATGTGCT
CCCTGGTGTGTGTTTTTGAATGTGTGTGAGATGGGTTGTCTCTCGGGACCTGGCAGGTGCGG
CCACCAGGTCAGGGCTGCCCCCCAACCCTGTGCCTCCTTCCTCCTAGACTCTGGCCCCCTCA
GTGCTGAGGGTGATACAGAGCACTTTTCAAGCTGGATTTGGAATGTGGCCTCTCCCCTCCAA
ACTCCTGGAGATCATGCAAAGGCCTTTGGAGCCAGCCAGTCACCTGGAAGGTGACATTCCCA
CCAGCTGAGGCCTCACCTTCAGCGGGGGCTGGGCAGCTTTGGAGCCTGGGGCCAGCCAAGCT
CACTCTGCCCATATCCCTGCCACGTGTGGCCCAGCGGATGATCACCTGTCTTCATCTGCGTA
CTGGGCCACATCCCTCCTGCCGTCCCCCACTTCCCTGATGACACCTACAGCAAGCCCCTACC
CAAGTGTTCTGTGATCCCCTGTAAATGTGGCCTCCCTAGCTACTTGCTTTTATGAAACCAAC
AATCCTGGGGACACAGTTTTCGGCTGTCTCAAGACGGGGCAACCACTCTTTTCCCCAGGCCT
GTGGGTCCCAGGCCTGGAGCTAGGGTTGGCATTCTTGCCTGAATTCTCCACTCTATCCCAAC
CCCTGAGGCCGCCTGAGGAGGCTCAGACTGTGTCAGGCTAGGAGGACAGTCAAACCACAAAA
ACATGCCTTTTAAGAAGTATAAGCACAAATCCCTCTTTGATGTTATATAAAAGCTCAGTGTC
ACTTTAATTAAGACCTCGAAGGGGACTTGGGGGGTTCGGGGCTTTCGGGGGCGGTCGGGGGT
TCGCGGACCCGGGAAGCTCTGAGGACCCAGAGGCCGGGCGCGCTCCGCCCGCGGCGCCGCCC
CCTCCGTAACTTTCCCAGTCTCCGAGGGAAGAGGCGGGGTGTGGGGTGCGGTTAAAAGGCGC
CACGGCGGGAGACAGGTGTTGCGGCCCCGCAGCGCCCGCGCGCTCCTCTCCCCGACTCGGAG
CCCCTCGGCGGCGCCCGGCCCAGGACCCGCCTAGGAGCGCAGGAGCCCCAGCGCAGAGACCC
CAACGCCGAGACCCCCGCCCCGGCCCCGCCGCGCTTCCTCCCGACGCAGAGCAAACCGCCCA
GAGTAGAAGCCATGGATTGGGGCACGCTGCAGACGATCCTGGGGGGTGTGAACAAACACTCC
ACCAGCATTGGAAAGATCTGGCTCACCGTCCTCTTCATTTTTCGCATTATGATCCTCGTTGT
GGCTGCAAAGGAGGTGTGGGGAGATGAGCAGGCCGACTTTGTCTGCAACACCCTGCAGCCAG
GCTGCAAGAACGTGTGCTACGATCACTACTTCCCCATCTCCCACATCCGGCTATGGGCCCTG
CAGCTGATCTTCGTGTCCACGCCAGCGCTCCTAGTGGCCATGCACGTGGCCTACCGGAGACA
TGAGAAGAAGAGGAAGTTCATCAAGGGGGAGATAAAGAGTGAATTTAAGGACATCGAGGAGA
TCAAAACCCAGAAGGTCCGCATCGAAGGCTCCCTGTGGTGGACCTACACAAGCAGCATCTTC
TTCCGGGTCATCTTCGAAGCCGCCTTCATGTACGTCTTCTATGTCATGTACGACGGCTTCTC
CATGCAGCGGCTGGTGAAGTGCAACGCCTGGCCTTGTCCCAACACTGTGGACTGCTTTGTGT
CCCGGCCCACGGAGAAGACTGTCTTCACAGTGTTCATGATTGCAGTGTCTGGAATTTGCATC
CTGCTGAATGTCACTGAATTGTGTTATTTGCTAATTAGATATTGTTCTGGGAAGTCAAAAAA
GCCAGTTTAAAGGCGCGCCACCCCTGCAGGGAATTCCGCATTGCCCAGTTGTTAGATTAAGA
AATAGACAGCATGAGAGGGATGAGGCAACCCGTGCTCAGCTGTCAAGGCTCAGTCGCTAGCA
TTTCCCAACACAAAGATTCTGACCTTAAATGCAACCATTTGAAACCCCTGTAGGCCTCAGGT
GAAACTCCAGATGCCACAATGGAGCTCTGCTCCCCTAAAGCCTCAAAACAAAGGCCTAATTC
TATGCCTGTCTTAATTTTCTTTCACTTAAGTTAGTTCCACTGAGACCCCAGGCTGTTAGGGG
TTATTGGTGTAAGGTACTTTCATATTTTAAACAGAGGATATCGGCATTTGTTTCTTTCTCTG
AGGACAAGAGAAAAAAGCCAGGTTCCACAGAGGACACAGAGAAGGTTTGGGTGTCCTCCTGG
GGTTCTTTTTGCCAACTTTCCCCACGTTAAAGGTGAACATTGGTTCTTTCATTTGCTTTGGA
AGTTTTAATCTCTAACAGTGGACAAAGTTACCAGTGCCTTAAACTCTGTTACACTTTTTGGA
AGTGAAAACTTTGTAGTATGATAGGTTATTTTGATGTAAAGATGTTCTGGATACCATTATAT
GTTCCCCCTGTTTCAGAGGCTCAGATTGTAATATGTAAATGGTATGTCATTCGCTACTATGA
TTTAATTTGAAATATGGTCTTTTGGTTATGAATACTTTGCAGCACAGCTGAGAGGCTGTCTG
TTGTATTCATTGTGGTCATAGCACCTAACAACATTGTAGCCTCAATCGAGTGAGACAGACTA
GAAGTTCCTAGTGATGGCTTATGATAGCAAATGGCCTCATGTCAAATATTTAGATGTAATTT
TGTGTAAGAAATACAGACTGGATGTACCACCAACTACTACCTGTAATGACAGGCCTGTCCAA
CACATCTCCCTTTTCCATGACTGTGGTAGCCAGCATCGGAAAGAACGCTGATTTAAAGAGGT
CGCTTGGGAATTTTATTGACACAGTACCATTTAATGGGGAGGACAAAATGGGGCAGGGGAGG
GAGAAGTTTCTGTCGTTAAAAACAGATTTGGAAAGACTGGACTCTAAAGTCTGTTGATTAAA
GATGAGCTTTGTCTACTTCAAAAGTTTGTTTGCTTACCCCTTCAGCCTCCAATTTTTTAAGT
GAAAATATAGCTAATAACATGTGAAAAGAATAGAAGCTAAGGTTTAGATAAATATTGAGCAG
ATCTATAGGAAGATTGAACCTGAATATTGCCATTATGCTTGACATGGTTTCCAAAAAATGGT
ACTCCACATATTTCAGTGAGGGTAAGTATTTTCCTGTTGTCAAGAATAGCATTGTAAAAGCA
TTTTGTAATAATAAAGAATAGCTTTAATGATATGCTTGTAACTAAAATAATTTTGTAATGTA
TCAAATACATTTAAAACATTAAAATATAATCTCTATAATAATTTAAAATCTAATATGGTTTT
AATAGAACAGCGATATCAAGCTTATCGATAATCAACCTCTGGATTACAAAATTTGTGAAAGA
TTGACTGGTATTCTTAACTATGTTGCTCCTTTTACGCTATGTGGATACGCTGCTTTAATGCC
TTTGTATCATGCTATTGCTTCCCGTATGGCTTTCATTTTCTCCTCCTTGTATAAATCCTGGT
TGCTGTCTCTTTATGAGGAGTTGTGGCCCGTTGTCAGGCAACGTGGCGTGGTGTGCACTGTG
TTTGCTGACGCAACCCCCACTGGTTGGGGCATTGCCACCACCTGTCAGCTCCTTTCCGGGAC
TTTCGCTTTCCCCCTCCCTATTGCCACGGCGGAACTCATCGCCGCCTGCCTTGCCCGCTGCT
GGACAGGGGCTCGGCTGTTGGGCACTGACAATTCCGTGGTGTTGTCGGGGAAATCATCGTCC
TTTCCTTGGCTGCTCGCCTATGTTGCCACCTGGATTCTGCGCGGGACGTCCTTCTGCTACGT
CCCTTCGGCCCTCAATCCAGCGGACCTTCCTTCCCGCGGCCTGCTGCCGGCTCTGCGGCCTC
TTCCGCGTCTTCGCCTTCGCCCTCAGACGAGTCGGATCTCCCTTTGGGCCGCCTCCCCGCGA
ATTCATCGATACCGAGCGCTGCTCGAGAGATCTGTGATAGCGGCCATCAAGCTGGCTGTGCC
TTCTAGTTGCCAGCCATCTGTTGTTTGCCCCTCCCCCGTGCCTTCCTTGACCCTGGAAGGTG
CCACTCCCACTGTCCTTTCCTAATAAAATGAGGAAATTGCATCGCATTGTCTGAGTAGGTGT
CATTCTATTCTGGGGGGTGGGGTGGGGCAGGACAGCAAGGGGGAGGATTGGGAAGACAATAG
CAGGCATGCTGGGGACACGTGCGGACCGAGCGGCCGCAGGAACCCCTAGTGATGGAGTTGGC
CACTCCCTCTCTGCGCGCTCGCTCGCTCACTGAGGCCGGGCGACCAAAGGTCGCCCGACGCC
CGGGCTTTGCCCGGGCGGCCTCAGTGAGCGAGCGAGCGCGCAGCTGCCTGCAGGGGCGCCTG
ATGCGGTATTTTCTCCTTACGCATCTGTGCGGTATTTCACACCGCATACGTCAAAGCAACCA
TAGTACGCGCCCTGTAGCGGCGCATTAAGCGCGGCGGGTGTGGTGGTTACGCGCAGCGTGAC
CGCTACACTTGCCAGCGCCCTAGCGCCCGCTCCTTTCGCTTTCTTCCCTTCCTTTCTCGCCA
CGTTCGCCGGCTTTCCCCGTCAAGCTCTAAATCGGGGGCTCCCTTTAGGGTTCCGATTTAGT
GCTTTACGGCACCTCGACCCCAAAAAACTTGATTTGGGTGATGGTTCACGTAGTGGGCCATC
GCCCTGATAGACGGTTTTTCGCCCTTTGACGTTGGAGTCCACGTTCTTTAATAGTGGACTCT
TGTTCCAAACTGGAACAACACTCAACCCTATCTCGGGCTATTCTTTTGATTTATAAGGGATT
TTGCCGATTTCGGCCTATTGGTTAAAAAATGAGCTGATTTAACAAAAATTTAACGCGAATTT
TAACAAAATATTAACGTTTACAATTTTATGGTGCACTCTCAGTACAATCTGCTCTGATGCCG
CATAGTTAAGCCAGCCCCGACACCCGCCAACACCCGCTGACGCGCCCTGACGGGCTTGTCTG
CTCCCGGCATCCGCTTACAGACAAGCTGTGACCGTCTCCGGGAGCTGCATGTGTCAGAGGTT
TTCACCGTCATCACCGAAACGCGCGAGACGAAAGGGCCTCGTGATACGCCTATTTTTATAGG
TTAATGTCATGATAATAATGGTTTCTTAGACGTCAGGTGGCACTTTTCGGGGAAATGTGCGC
GGAACCCCTATTTGTTTATTTTTCTAAATACATTCAAATATGTATCCGCTCATGAGACAATA
ACCCTGATAAATGCTTCAATAATATTGAAAAAGGAAGAGTATGAGTATTCAACATTTCCGTG
TCGCCCTTATTCCCTTTTTTGCGGCATTTTGCCTTCCTGTTTTTGCTCACCCAGAAACGCTG
GTGAAAGTAAAAGATGCTGAAGATCAGTTGGGTGCACGAGTGGGTTACATCGAACTGGATCT
CAACAGCGGTAAGATCCTTGAGAGTTTTCGCCCCGAAGAACGTTTTCCAATGATGAGCACTT
TTAAAGTTCTGCTATGTGGCGCGGTATTATCCCGTATTGACGCCGGGCAAGAGCAACTCGGT
CGCCGCATACACTATTCTCAGAATGACTTGGTTGAGTACTCACCAGTCACAGAAAAGCATCT
TACGGATGGCATGACAGTAAGAGAATTATGCAGTGCTGCCATAACCATGAGTGATAACACTG
CGGCCAACTTACTTCTGACAACGATCGGAGGACCGAAGGAGCTAACCGCTTTTTTGCACAAC
ATGGGGGATCATGTAACTCGCCTTGATCGTTGGGAACCGGAGCTGAATGAAGCCATACCAAA
CGACGAGCGTGACACCA

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 81. An exemplary nucleotide sequence for vector c.81.8 is set forth in SEQ ID NO: 81 (hGJB2 GRE8 underlined; mouse GJB2 coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTC
GACCTGAACGATTAAGGCAAAACTTCGAAATGTGCCCCAGCAGAGATTTATTTTTCAGGGGG
TGTTTTGCATTCCAGCCCCTCTGCCTTCCTGGCGTTTAGTGCGATTTGTTTAGCCATGTGCT
CCCTGGTGTGTGTTTTTGAATGTGTGTGAGATGGGTTGTCTCTCGGGACCTGGCAGGTGCGG
CCACCAGGTCAGGGCTGCCCCCCAACCCTGTGCCTCCTTCCTCCTAGACTCTGGCCCCCTCA
GTGCTGAGGGTGATACAGAGCACTTTTCAAGCTGGATTTGGAATGTGGCCTCTCCCCTCCAA
ACTCCTGGAGATCATGCAAAGGCCTTTGGAGCCAGCCAGTCACCTGGAAGGTGACATTCCCA
CCAGCTGAGGCCTCACCTTCAGCGGGGGCTGGGCAGCTTTGGAGCCTGGGGCCAGCCAAGCT
CACTCTGCCCATATCCCTGCCACGTGTGGCCCAGCGGATGATCACCTGTCTTCATCTGCGTA
CTGGGCCACATCCCTCCTGCCGTCCCCCACTTCCCTGATGACACCTACAGCAAGCCCCTACC
CAAGTGTTCTGTGATCCCCTGTAAATGTGGCCTCCCTAGCTACTTGCTTTTATGAAACCAAC
AATCCTGGGGACACAGTTTTCGGCTGTCTCAAGACGGGGCAACCACTCTTTTCCCCAGGCCT
GTGGGTCCCAGGCCTGGAGCTAGGGTTGGCATTCTTGCCTGAATTCTCCACTCTATCCCAAC
CCCTGAGGCCGCCTGAGGAGGCTCAGACTGTGTCAGGCTAGGAGGACAGTCAAACCACAAAA
ACATGCCTTTTAAGAAGTATAAGCACAAATCCCTCTTTGATGTTATATAAAAGCTCAGTGTC
ACTTTAATTAAGACCTCGAAGGGGACTTGGGGGGTTCGGGGCTTTCGGGGGCGGTCGGGGGT
TCGCGGACCCGGGAAGCTCTGAGGACCCAGAGGCCGGGCGCGCTCCGCCCGCGGCGCCGCCC
CCTCCGTAACTTTCCCAGTCTCCGAGGGAAGAGGCGGGGTGTGGGGTGCGGTTAAAAGGCGC
CACGGCGGGAGACAGGTGTTGCGGCCCCGCAGCGCCCGCGCGCTCCTCTCCCCGACTCGGAG
CCCCTCGGCGGCGCCCGGCCCAGGACCCGCCTAGGAGCGCAGGAGCCCCAGCGCAGAGACCC
CAACGCCGAGACCCCCGCCCCGGCCCCGCCGCGCTTCCTCCCGACGCAGAGCAAACCGCCCA
GAGTAGAAGCCATGGATTGGGGCACACTCCAGAGCATCCTCGGGGGTGTCAACAAACACTCC
ACCAGCATTGGAAAGATCTGGCTCACGGTCCTCTTCATCTTCCGCATCATGATCCTCGTGGT
GGCTGCAAAGGAGGTGTGGGGAGATGAGCAAGCCGATTTTGTCTGCAACACGCTCCAGCCTG
GCTGCAAGAATGTATGCTACGACCACCACTTCCCCATCTCTCACATCCGGCTCTGGGCTCTG
CAGCTGATCATGGTGTCCACGCCAGCCCTCCTGGTAGCTATGCATGTGGCCTACCGGAGACA
TGAAAAGAAACGGAAGTTCATGAAGGGAGAGATAAAGAACGAGTTTAAGGACATCGAAGAGA
TCAAAACCCAGAAGGTCCGTATCGAAGGGTCCCTGTGGTGGACCTACACCACCAGCATCTTC
TTCCGGGTCATCTTTGAAGCCGTCTTCATGTACGTCTTTTACATCATGTACAATGGCTTCTT
CATGCAACGTCTGGTGAAATGCAACGCTTGGCCCTGCCCCAATACAGTGGACTGCTTCATTT
CCAGGCCCACAGAAAAGACTGTCTTCACCGTGTTTATGATTTCTGTGTCTGGAATTTGCATT
CTGCTAAATATCACAGAGCTGTGCTATTTGTTCGTTAGGTATTGCTCAGGAAAGTCCAAAAG
ACCAGTCTACCCATACGATGTTCCAGATTACGCTTAAAGGCGCGCCACCCCTGCAGGGAATT
CCGCATTGCCCAGTTGTTAGATTAAGAAATAGACAGCATGAGAGGGATGAGGCAACCCGTGC
TCAGCTGTCAAGGCTCAGTCGCTAGCATTTCCCAACACAAAGATTCTGACCTTAAATGCAAC
CATTTGAAACCCCTGTAGGCCTCAGGTGAAACTCCAGATGCCACAATGGAGCTCTGCTCCCC
TAAAGCCTCAAAACAAAGGCCTAATTCTATGCCTGTCTTAATTTTCTTTCACTTAAGTTAGT
TCCACTGAGACCCCAGGCTGTTAGGGGTTATTGGTGTAAGGTACTTTCATATTTTAAACAGA
GGATATCGGCATTTGTTTCTTTCTCTGAGGACAAGAGAAAAAAGCCAGGTTCCACAGAGGAC
ACAGAGAAGGTTTGGGTGTCCTCCTGGGGTTCTTTTTGCCAACTTTCCCCACGTTAAAGGTG
AACATTGGTTCTTTCATTTGCTTTGGAAGTTTTAATCTCTAACAGTGGACAAAGTTACCAGT
GCCTTAAACTCTGTTACACTTTTTGGAAGTGAAAACTTTGTAGTATGATAGGTTATTTTGAT
GTAAAGATGTTCTGGATACCATTATATGTTCCCCCTGTTTCAGAGGCTCAGATTGTAATATG
TAAATGGTATGTCATTCGCTACTATGATTTAATTTGAAATATGGTCTTTTGGTTATGAATAC
TTTGCAGCACAGCTGAGAGGCTGTCTGTTGTATTCATTGTGGTCATAGCACCTAACAACATT
GTAGCCTCAATCGAGTGAGACAGACTAGAAGTTCCTAGTGATGGCTTATGATAGCAAATGGC
CTCATGTCAAATATTTAGATGTAATTTTGTGTAAGAAATACAGACTGGATGTACCACCAACT
ACTACCTGTAATGACAGGCCTGTCCAACACATCTCCCTTTTCCATGACTGTGGTAGCCAGCA
TCGGAAAGAACGCTGATTTAAAGAGGTCGCTTGGGAATTTTATTGACACAGTACCATTTAAT
GGGGAGGACAAAATGGGGCAGGGGAGGGAGAAGTTTCTGTCGTTAAAAACAGATTTGGAAAG
ACTGGACTCTAAAGTCTGTTGATTAAAGATGAGCTTTGTCTACTTCAAAAGTTTGTTTGCTT
ACCCCTTCAGCCTCCAATTTTTTAAGTGAAAATATAGCTAATAACATGTGAAAAGAATAGAA
GCTAAGGTTTAGATAAATATTGAGCAGATCTATAGGAAGATTGAACCTGAATATTGCCATTA
TGCTTGACATGGTTTCCAAAAAATGGTACTCCACATATTTCAGTGAGGGTAAGTATTTTCCT
GTTGTCAAGAATAGCATTGTAAAAGCATTTTGTAATAATAAAGAATAGCTTTAATGATATGC
TTGTAACTAAAATAATTTTGTAATGTATCAAATACATTTAAAACATTAAAATATAATCTCTA
TAATAATTTAAAATCTAATATGGTTTTAATAGAACAGCGATATCAAGCTTATCGATAATCAA
CCTCTGGATTACAAAATTTGTGAAAGATTGACTGGTATTCTTAACTATGTTGCTCCTTTTAC
GCTATGTGGATACGCTGCTTTAATGCCTTTGTATCATGCTATTGCTTCCCGTATGGCTTTCA
TTTTCTCCTCCTTGTATAAATCCTGGTTGCTGTCTCTTTATGAGGAGTTGTGGCCCGTTGTC
AGGCAACGTGGCGTGGTGTGCACTGTGTTTGCTGACGCAACCCCCACTGGTTGGGGCATTGC
CACCACCTGTCAGCTCCTTTCCGGGACTTTCGCTTTCCCCCTCCCTATTGCCACGGCGGAAC
TCATCGCCGCCTGCCTTGCCCGCTGCTGGACAGGGGCTCGGCTGTTGGGCACTGACAATTCC
GTGGTGTTGTCGGGGAAATCATCGTCCTTTCCTTGGCTGCTCGCCTATGTTGCCACCTGGAT
TCTGCGCGGGACGTCCTTCTGCTACGTCCCTTCGGCCCTCAATCCAGCGGACCTTCCTTCCC
GCGGCCTGCTGCCGGCTCTGCGGCCTCTTCCGCGTCTTCGCCTTCGCCCTCAGACGAGTCGG
ATCTCCCTTTGGGCCGCCTCCCCGCGAATTCATCGATACCGAGCGCTGCTCGAGAGATCTGT
GATAGCGGCCATCAAGCTGGCTGTGCCTTCTAGTTGCCAGCCATCTGTTGTTTGCCCCTCCC
CCGTGCCTTCCTTGACCCTGGAAGGTGCCACTCCCACTGTCCTTTCCTAATAAAATGAGGAA
ATTGCATCGCATTGTCTGAGTAGGTGTCATTCTATTCTGGGGGGTGGGGTGGGGCAGGACAG
CAAGGGGGAGGATTGGGAAGACAATAGCAGGCATGCTGGGGACACGTGCGGACCGAGCGGCC
GCAGGAACCCCTAGTGATGGAGTTGGCCACTCCCTCTCTGCGCGCTCGCTCGCTCACTGAGG
CCGGGCGACCAAAGGTCGCCCGACGCCCGGGCTTTGCCCGGGCGGCCTCAGTGAGCGAGCGA
GCGCGCAGCTGCCTGCAGGGGCGCCTGATGCGGTATTTTCTCCTTACGCATCTGTGCGGTAT
TTCACACCGCATACGTCAAAGCAACCATAGTACGCGCCCTGTAGCGGCGCATTAAGCGCGGC
GGGTGTGGTGGTTACGCGCAGCGTGACCGCTACACTTGCCAGCGCCCTAGCGCCCGCTCCTT
TCGCTTTCTTCCCTTCCTTTCTCGCCACGTTCGCCGGCTTTCCCCGTCAAGCTCTAAATCGG
GGGCTCCCTTTAGGGTTCCGATTTAGTGCTTTACGGCACCTCGACCCCAAAAAACTTGATTT
GGGTGATGGTTCACGTAGTGGGCCATCGCCCTGATAGACGGTTTTTCGCCCTTTGACGTTGG
AGTCCACGTTCTTTAATAGTGGACTCTTGTTCCAAACTGGAACAACACTCAACCCTATCTCG
GGCTATTCTTTTGATTTATAAGGGATTTTGCCGATTTCGGCCTATTGGTTAAAAAATGAGCT
GATTTAACAAAAATTTAACGCGAATTTTAACAAAATATTAACGTTTACAATTTTATGGTGCA
CTCTCAGTACAATCTGCTCTGATGCCGCATAGTTAAGCCAGCCCCGACACCCGCCAACACCC
GCTGACGCGCCCTGACGGGCTTGTCTGCTCCCGGCATCCGCTTACAGACAAGCTGTGACCGT
CTCCGGGAGCTGCATGTGTCAGAGGTTTTCACCGTCATCACCGAAACGCGCGAGACGAAAGG
GCCTCGTGATACGCCTATTTTTATAGGTTAATGTCATGATAATAATGGTTTCTTAGACGTCA
GGTGGCACTTTTCGGGGAAATGTGCGCGGAACCCCTATTTGTTTATTTTTCTAAATACATTC
AAATATGTATCCGCTCATGAGACAATAACCCTGATAAATGCTTCAATAATATTGAAAAAGGA
AGAGTATGAGTATTCAACATTTCCGTGTCGCCCTTATTCCCTTTTTTGCGGCATTTTGCCTT
CCTGTTTTTGCTCACCCAGAAACGCTGGTGAAAGTAAAAGATGCTGAAGATCAGTTGGGTGC
ACGAGTGGGTTACATCGAACTGGATCTCAACAGCGGTAAGATCCTTGAGAGTTTTCGCCCCG
AAGAACGTTTTCCAATGATGAGCACTTTTAAAGTTCTGCTATGTGGCGCGGTATTATCCCGT
ATTGACGCCGGGCAAGAGCAACTCGGTCGCCGCATACACTATTCTCAGAATGACTTGGTTGA
GTACTCACCAGTCACAGAAAAGCATCTTACGGATGGCATGACAGTAAGAGAATTATGCAGTG
CTGCCATAACCATGAGTGATAACACTGCGGCCAACTTACTTCTGACAACGATCGGAGGACCG
AAGGAGCTAACCGCTTTTTTGCACAACATGGGGGATCATGTAACTCGCCTTGATCGTTGGGA
ACCGGAGCTGAATGAAGCCATACCAAACGACGAGCGTGACACCA

In some embodiments, an AAV vector described herein comprises an AAV 5′ ITR, a GJB2 GRE enhancer (hGJB2 GRE9), a GJB2 basal promoter, a GJB2 exon 1 5′ UTR, Kozak sequence, nucleotide sequence encoding a gene product (e.g., GJB2 or GFP), a GJB2 exon 2 3′ UTR, a WPRE, a bovine growth hormone poly A signal, and an AAV 3′ ITR (e.g., vector c.81.9).

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 52. An exemplary nucleotide sequence for vector c.81.9 is set forth in SEQ ID NO: 52 (hGJB2 GRE9 underlined; eGFP coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTT
CTAGGTAGACAACTAAGATGTTCATCTTATGGTTTAATGTTTAGTTGTAAAGGTTGTTTGCT
TCTCATTTGGTTCCAAGAAAGAGTATTTAGGCCAATTTCAGGGAGAAATATGTGTATAGATA
TATTCATATGTCAAACTGATTAGTGCTGAATGTCACATTTCCATATTCTAATAACATTTCTA
GCAAAGAAGAGGACACAGTGAAGAGAGAATTGCCCGCATTGTCATTGTCTCTTTCTGAGCCT
AGAACGCCTAACACTTGGGTGTGGAGAGACTCAGCCTCAATTCACTTTCTAGCAGCCACTGA
GATGTGCTTGCCTGGGGTGCCCCCTGGCAGGCAGGGCTGGAACTGCTTTCCAGTACCCACAC
GGACTGTGAACGAATCTTTCTTTGTGCTTTGTGTACAGAATGGAAGTTCAACAAATATTTGT
TGAATGTGTATGTCCTTCCAATACGCAGCAGCCCAGAGCAAACGTGGTAATCTTGTGTGTGT
TCATGTGAAAGCAGAATTTAATGGTGCTTTTAAGCACCAAAGTTTAAGATGCACGAGAAAAC
TGTATCTCCATTTTTTCCTTTTCGTTTACAATTACTTGTATAAGCCAGGCACGGTGGTGGCT
CACGCCTGTAATCCCAGCACTTTGGGAGGCCGAGGCGGGCGGATCACATGAGGTCGGGAGTT
AATTAAGACCTCGAAGGGGACTTGGGGGGTTCGGGGCTTTCGGGGGCGGTCGGGGGTTCGCG
GACCCGGGAAGCTCTGAGGACCCAGAGGCCGGGCGCGCTCCGCCCGCGGCGCCGCCCCCTCC
GTAACTTTCCCAGTCTCCGAGGGAAGAGGCGGGGTGTGGGGTGCGGTTAAAAGGCGCCACGG
CGGGAGACAGGTGTTGCGGCCCCGCAGCGCCCGCGCGCTCCTCTCCCCGACTCGGAGCCCCT
CGGCGGCGCCCGGCCCAGGACCCGCCTAGGAGCGCAGGAGCCCCAGCGCAGAGACCCCAACG
CCGAGACCCCCGCCCCGGCCCCGCCGCGCTTCCTCCCGACGCAGAGCAAACCGCCCAGAGTA
GAAGCCATGGTGAGCAAGGGCGAGGAGCTGTTCACCGGGGTGGTGCCCATCCTGGTCGAGCT
GGACGGCGACGTAAACGGCCACAAGTTCAGCGTGTCCGGCGAGGGCGAGGGCGATGCCACCT
ACGGCAAGCTGACCCTGAAGTTCATCTGCACCACCGGCAAGCTGCCCGTGCCCTGGCCCACC
CTCGTGACCACCCTGACCTACGGCGTGCAGTGCTTCAGCCGCTACCCCGACCACATGAAGCA
GCACGACTTCTTCAAGTCCGCCATGCCCGAAGGCTACGTCCAGGAGCGCACCATCTTCTTCA
AGGACGACGGCAACTACAAGACCCGCGCCGAGGTGAAGTTCGAGGGCGACACCCTGGTGAAC
CGCATCGAGCTGAAGGGCATCGACTTCAAGGAGGACGGCAACATCCTGGGGCACAAGCTGGA
GTACAACTACAACAGCCACAACGTCTATATCATGGCCGACAAGCAGAAGAACGGCATCAAGG
TGAACTTCAAGATCCGCCACAACATCGAGGACGGCAGCGTGCAGCTCGCCGACCACTACCAG
CAGAACACCCCCATCGGCGACGGCCCCGTGCTGCTGCCCGACAACCACTACCTGAGCACCCA
GTCCGCCCTGAGCAAAGACCCCAACGAGAAGCGCGATCACATGGTCCTGCTGGAGTTCGTGA
CCGCCGCCGGGATCACTCTCGGCATGGACGAGCTGTACAAGTAAAGGCGCGCCACCCCTGCA
GGGAATTCCGCATTGCCCAGTTGTTAGATTAAGAAATAGACAGCATGAGAGGGATGAGGCAA
CCCGTGCTCAGCTGTCAAGGCTCAGTCGCTAGCATTTCCCAACACAAAGATTCTGACCTTAA
ATGCAACCATTTGAAACCCCTGTAGGCCTCAGGTGAAACTCCAGATGCCACAATGGAGCTCT
GCTCCCCTAAAGCCTCAAAACAAAGGCCTAATTCTATGCCTGTCTTAATTTTCTTTCACTTA
AGTTAGTTCCACTGAGACCCCAGGCTGTTAGGGGTTATTGGTGTAAGGTACTTTCATATTTT
AAACAGAGGATATCGGCATTTGTTTCTTTCTCTGAGGACAAGAGAAAAAAGCCAGGTTCCAC
AGAGGACACAGAGAAGGTTTGGGTGTCCTCCTGGGGTTCTTTTTGCCAACTTTCCCCACGTT
AAAGGTGAACATTGGTTCTTTCATTTGCTTTGGAAGTTTTAATCTCTAACAGTGGACAAAGT
TACCAGTGCCTTAAACTCTGTTACACTTTTTGGAAGTGAAAACTTTGTAGTATGATAGGTTA
TTTTGATGTAAAGATGTTCTGGATACCATTATATGTTCCCCCTGTTTCAGAGGCTCAGATTG
TAATATGTAAATGGTATGTCATTCGCTACTATGATTTAATTTGAAATATGGTCTTTTGGTTA
TGAATACTTTGCAGCACAGCTGAGAGGCTGTCTGTTGTATTCATTGTGGTCATAGCACCTAA
CAACATTGTAGCCTCAATCGAGTGAGACAGACTAGAAGTTCCTAGTGATGGCTTATGATAGC
AAATGGCCTCATGTCAAATATTTAGATGTAATTTTGTGTAAGAAATACAGACTGGATGTACC
ACCAACTACTACCTGTAATGACAGGCCTGTCCAACACATCTCCCTTTTCCATGACTGTGGTA
GCCAGCATCGGAAAGAACGCTGATTTAAAGAGGTCGCTTGGGAATTTTATTGACACAGTACC
ATTTAATGGGGAGGACAAAATGGGGCAGGGGAGGGAGAAGTTTCTGTCGTTAAAAACAGATT
TGGAAAGACTGGACTCTAAAGTCTGTTGATTAAAGATGAGCTTTGTCTACTTCAAAAGTTTG
TTTGCTTACCCCTTCAGCCTCCAATTTTTTAAGTGAAAATATAGCTAATAACATGTGAAAAG
AATAGAAGCTAAGGTTTAGATAAATATTGAGCAGATCTATAGGAAGATTGAACCTGAATATT
GCCATTATGCTTGACATGGTTTCCAAAAAATGGTACTCCACATATTTCAGTGAGGGTAAGTA
TTTTCCTGTTGTCAAGAATAGCATTGTAAAAGCATTTTGTAATAATAAAGAATAGCTTTAAT
GATATGCTTGTAACTAAAATAATTTTGTAATGTATCAAATACATTTAAAACATTAAAATATA
ATCTCTATAATAATTTAAAATCTAATATGGTTTTAATAGAACAGCGATATCAAGCTTATCGA
TAATCAACCTCTGGATTACAAAATTTGTGAAAGATTGACTGGTATTCTTAACTATGTTGCTC
CTTTTACGCTATGTGGATACGCTGCTTTAATGCCTTTGTATCATGCTATTGCTTCCCGTATG
GCTTTCATTTTCTCCTCCTTGTATAAATCCTGGTTGCTGTCTCTTTATGAGGAGTTGTGGCC
CGTTGTCAGGCAACGTGGCGTGGTGTGCACTGTGTTTGCTGACGCAACCCCCACTGGTTGGG
GCATTGCCACCACCTGTCAGCTCCTTTCCGGGACTTTCGCTTTCCCCCTCCCTATTGCCACG
GCGGAACTCATCGCCGCCTGCCTTGCCCGCTGCTGGACAGGGGCTCGGCTGTTGGGCACTGA
CAATTCCGTGGTGTTGTCGGGGAAATCATCGTCCTTTCCTTGGCTGCTCGCCTATGTTGCCA
CCTGGATTCTGCGCGGGACGTCCTTCTGCTACGTCCCTTCGGCCCTCAATCCAGCGGACCTT
CCTTCCCGCGGCCTGCTGCCGGCTCTGCGGCCTCTTCCGCGTCTTCGCCTTCGCCCTCAGAC
GAGTCGGATCTCCCTTTGGGCCGCCTCCCCGCGAATTCATCGATACCGAGCGCTGCTCGAGA
GATCTGTGATAGCGGCCATCAAGCTGGCTGTGCCTTCTAGTTGCCAGCCATCTGTTGTTTGC
CCCTCCCCCGTGCCTTCCTTGACCCTGGAAGGTGCCACTCCCACTGTCCTTTCCTAATAAAA
TGAGGAAATTGCATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGACACGTGCGGACCG
AGCGGCCGCAGGAACCCCTAGTGATGGAGTTGGCCACTCCCTCTCTGCGCGCTCGCTCGCTC
ACTGAGGCCGGGCGACCAAAGGTCGCCCGACGCCCGGGCTTTGCCCGGGCGGCCTCAGTGAG
CGAGCGAGCGCGCAGCTGCCTGCAGGGGCGCCTGATGCGGTATTTTCTCCTTACGCATCTGT
GCGGTATTTCACACCGCATACGTCAAAGCAACCATAGTACGCGCCCTGTAGCGGCGCATTAA
GCGCGGCGGGTGTGGTGGTTACGCGCAGCGTGACCGCTACACTTGCCAGCGCCCTAGCGCCC
GCTCCTTTCGCTTTCTTCCCTTCCTTTCTCGCCACGTTCGCCGGCTTTCCCCGTCAAGCTCT
AAATCGGGGGCTCCCTTTAGGGTTCCGATTTAGTGCTTTACGGCACCTCGACCCCAAAAAAC
TTGATTTGGGTGATGGTTCACGTAGTGGGCCATCGCCCTGATAGACGGTTTTTCGCCCTTTG
ACGTTGGAGTCCACGTTCTTTAATAGTGGACTCTTGTTCCAAACTGGAACAACACTCAACCC
TATCTCGGGCTATTCTTTTGATTTATAAGGGATTTTGCCGATTTCGGCCTATTGGTTAAAAA
ATGAGCTGATTTAACAAAAATTTAACGCGAATTTTAACAAAATATTAACGTTTACAATTTTA
TGGTGCACTCTCAGTACAATCTGCTCTGATGCCGCATAGTTAAGCCAGCCCCGACACCCGCC
AACACCCGCTGACGCGCCCTGACGGGCTTGTCTGCTCCCGGCATCCGCTTACAGACAAGCTG
TGACCGTCTCCGGGAGCTGCATGTGTCAGAGGTTTTCACCGTCATCACCGAAACGCGCGAGA
CGAAAGGGCCTCGTGATACGCCTATTTTTATAGGTTAATGTCATGATAATAATGGTTTCTTA
GACGTCAGGTGGCACTTTTCGGGGAAATGTGCGCGGAACCCCTATTTGTTTATTTTTCTAAA
TACATTCAAATATGTATCCGCTCATGAGACAATAACCCTGATAAATGCTTCAATAATATTGA
AAAAGGAAGAGTATGAGTATTCAACATTTCCGTGTCGCCCTTATTCCCTTTTTTGCGGCATT
TTGCCTTCCTGTTTTTGCTCACCCAGAAACGCTGGTGAAAGTAAAAGATGCTGAAGATCAGT
TGGGTGCACGAGTGGGTTACATCGAACTGGATCTCAACAGCGGTAAGATCCTTGAGAGTTTT
CGCCCCGAAGAACGTTTTCCAATGATGAGCACTTTTAAAGTTCTGCTATGTGGCGCGGTATT
ATCCCGTATTGACGCCGGGCAAGAGCAACTCGGTCGCCGCATACACTATTCTCAGAATGACT
TGGTTGAGTACTCACCAGTCACAGAAAAGCATCTTACGGATGGCATGACAGTAAGAGAATTA
TGCAGTGCTGCCATAACCATGAGTGATAACACTGCGGCCAACTTACTTCTGACAACGATCGG
AGGACCGAAGGAGCTAACCGCTTTTTTGCACAACATGGGGGATCATGTAACTCGCCTTGATC
GTTGGGAACCGGAGCTGAATGAAGCCATACCAAACGACGAGCGTGACACCA

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 82. An exemplary nucleotide sequence for vector c.81.9 is set forth in SEQ ID NO: 82 (hGJB2 GRE9 underlined; human GJB2 coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTT
CTAGGTAGACAACTAAGATGTTCATCTTATGGTTTAATGTTTAGTTGTAAAGGTTGTTTGCT
TCTCATTTGGTTCCAAGAAAGAGTATTTAGGCCAATTTCAGGGAGAAATATGTGTATAGATA
TATTCATATGTCAAACTGATTAGTGCTGAATGTCACATTTCCATATTCTAATAACATTTCTA
GCAAAGAAGAGGACACAGTGAAGAGAGAATTGCCCGCATTGTCATTGTCTCTTTCTGAGCCT
AGAACGCCTAACACTTGGGTGTGGAGAGACTCAGCCTCAATTCACTTTCTAGCAGCCACTGA
GATGTGCTTGCCTGGGGTGCCCCCTGGCAGGCAGGGCTGGAACTGCTTTCCAGTACCCACAC
GGACTGTGAACGAATCTTTCTTTGTGCTTTGTGTACAGAATGGAAGTTCAACAAATATTTGT
TGAATGTGTATGTCCTTCCAATACGCAGCAGCCCAGAGCAAACGTGGTAATCTTGTGTGTGT
TCATGTGAAAGCAGAATTTAATGGTGCTTTTAAGCACCAAAGTTTAAGATGCACGAGAAAAC
TGTATCTCCATTTTTTCCTTTTCGTTTACAATTACTTGTATAAGCCAGGCACGGTGGTGGCT
CACGCCTGTAATCCCAGCACTTTGGGAGGCCGAGGCGGGCGGATCACATGAGGTCGGGAGTT
AATTAAGACCTCGAAGGGGACTTGGGGGGTTCGGGGCTTTCGGGGGCGGTCGGGGGTTCGCG
GACCCGGGAAGCTCTGAGGACCCAGAGGCCGGGCGCGCTCCGCCCGCGGCGCCGCCCCCTCC
GTAACTTTCCCAGTCTCCGAGGGAAGAGGCGGGGTGTGGGGTGCGGTTAAAAGGCGCCACGG
CGGGAGACAGGTGTTGCGGCCCCGCAGCGCCCGCGCGCTCCTCTCCCCGACTCGGAGCCCCT
CGGCGGCGCCCGGCCCAGGACCCGCCTAGGAGCGCAGGAGCCCCAGCGCAGAGACCCCAACG
CCGAGACCCCCGCCCCGGCCCCGCCGCGCTTCCTCCCGACGCAGAGCAAACCGCCCAGAGTA
GAAGCCATGGATTGGGGCACGCTGCAGACGATCCTGGGGGGTGTGAACAAACACTCCACCAG
CATTGGAAAGATCTGGCTCACCGTCCTCTTCATTTTTCGCATTATGATCCTCGTTGTGGCTG
CAAAGGAGGTGTGGGGAGATGAGCAGGCCGACTTTGTCTGCAACACCCTGCAGCCAGGCTGC
AAGAACGTGTGCTACGATCACTACTTCCCCATCTCCCACATCCGGCTATGGGCCCTGCAGCT
GATCTTCGTGTCCACGCCAGCGCTCCTAGTGGCCATGCACGTGGCCTACCGGAGACATGAGA
AGAAGAGGAAGTTCATCAAGGGGGAGATAAAGAGTGAATTTAAGGACATCGAGGAGATCAAA
ACCCAGAAGGTCCGCATCGAAGGCTCCCTGTGGTGGACCTACACAAGCAGCATCTTCTTCCG
GGTCATCTTCGAAGCCGCCTTCATGTACGTCTTCTATGTCATGTACGACGGCTTCTCCATGC
AGCGGCTGGTGAAGTGCAACGCCTGGCCTTGTCCCAACACTGTGGACTGCTTTGTGTCCCGG
CCCACGGAGAAGACTGTCTTCACAGTGTTCATGATTGCAGTGTCTGGAATTTGCATCCTGCT
GAATGTCACTGAATTGTGTTATTTGCTAATTAGATATTGTTCTGGGAAGTCAAAAAAGCCAG
TTTAAAGGCGCGCCACCCCTGCAGGGAATTCCGCATTGCCCAGTTGTTAGATTAAGAAATAG
ACAGCATGAGAGGGATGAGGCAACCCGTGCTCAGCTGTCAAGGCTCAGTCGCTAGCATTTCC
CAACACAAAGATTCTGACCTTAAATGCAACCATTTGAAACCCCTGTAGGCCTCAGGTGAAAC
TCCAGATGCCACAATGGAGCTCTGCTCCCCTAAAGCCTCAAAACAAAGGCCTAATTCTATGC
CTGTCTTAATTTTCTTTCACTTAAGTTAGTTCCACTGAGACCCCAGGCTGTTAGGGGTTATT
GGTGTAAGGTACTTTCATATTTTAAACAGAGGATATCGGCATTTGTTTCTTTCTCTGAGGAC
AAGAGAAAAAAGCCAGGTTCCACAGAGGACACAGAGAAGGTTTGGGTGTCCTCCTGGGGTTC
TTTTTGCCAACTTTCCCCACGTTAAAGGTGAACATTGGTTCTTTCATTTGCTTTGGAAGTTT
TAATCTCTAACAGTGGACAAAGTTACCAGTGCCTTAAACTCTGTTACACTTTTTGGAAGTGA
AAACTTTGTAGTATGATAGGTTATTTTGATGTAAAGATGTTCTGGATACCATTATATGTTCC
CCCTGTTTCAGAGGCTCAGATTGTAATATGTAAATGGTATGTCATTCGCTACTATGATTTAA
TTTGAAATATGGTCTTTTGGTTATGAATACTTTGCAGCACAGCTGAGAGGCTGTCTGTTGTA
TTCATTGTGGTCATAGCACCTAACAACATTGTAGCCTCAATCGAGTGAGACAGACTAGAAGT
TCCTAGTGATGGCTTATGATAGCAAATGGCCTCATGTCAAATATTTAGATGTAATTTTGTGT
AAGAAATACAGACTGGATGTACCACCAACTACTACCTGTAATGACAGGCCTGTCCAACACAT
CTCCCTTTTCCATGACTGTGGTAGCCAGCATCGGAAAGAACGCTGATTTAAAGAGGTCGCTT
GGGAATTTTATTGACACAGTACCATTTAATGGGGAGGACAAAATGGGGCAGGGGAGGGAGAA
GTTTCTGTCGTTAAAAACAGATTTGGAAAGACTGGACTCTAAAGTCTGTTGATTAAAGATGA
GCTTTGTCTACTTCAAAAGTTTGTTTGCTTACCCCTTCAGCCTCCAATTTTTTAAGTGAAAA
TATAGCTAATAACATGTGAAAAGAATAGAAGCTAAGGTTTAGATAAATATTGAGCAGATCTA
TAGGAAGATTGAACCTGAATATTGCCATTATGCTTGACATGGTTTCCAAAAAATGGTACTCC
ACATATTTCAGTGAGGGTAAGTATTTTCCTGTTGTCAAGAATAGCATTGTAAAAGCATTTTG
TAATAATAAAGAATAGCTTTAATGATATGCTTGTAACTAAAATAATTTTGTAATGTATCAAA
TACATTTAAAACATTAAAATATAATCTCTATAATAATTTAAAATCTAATATGGTTTTAATAG
AACAGCGATATCAAGCTTATCGATAATCAACCTCTGGATTACAAAATTTGTGAAAGATTGAC
TGGTATTCTTAACTATGTTGCTCCTTTTACGCTATGTGGATACGCTGCTTTAATGCCTTTGT
ATCATGCTATTGCTTCCCGTATGGCTTTCATTTTCTCCTCCTTGTATAAATCCTGGTTGCTG
TCTCTTTATGAGGAGTTGTGGCCCGTTGTCAGGCAACGTGGCGTGGTGTGCACTGTGTTTGC
TGACGCAACCCCCACTGGTTGGGGCATTGCCACCACCTGTCAGCTCCTTTCCGGGACTTTCG
CTTTCCCCCTCCCTATTGCCACGGCGGAACTCATCGCCGCCTGCCTTGCCCGCTGCTGGACA
GGGGCTCGGCTGTTGGGCACTGACAATTCCGTGGTGTTGTCGGGGAAATCATCGTCCTTTCC
TTGGCTGCTCGCCTATGTTGCCACCTGGATTCTGCGCGGGACGTCCTTCTGCTACGTCCCTT
CGGCCCTCAATCCAGCGGACCTTCCTTCCCGCGGCCTGCTGCCGGCTCTGCGGCCTCTTCCG
CGTCTTCGCCTTCGCCCTCAGACGAGTCGGATCTCCCTTTGGGCCGCCTCCCCGCGAATTCA
TCGATACCGAGCGCTGCTCGAGAGATCTGTGATAGCGGCCATCAAGCTGGCTGTGCCTTCTA
GTTGCCAGCCATCTGTTGTTTGCCCCTCCCCCGTGCCTTCCTTGACCCTGGAAGGTGCCACT
CCCACTGTCCTTTCCTAATAAAATGAGGAAATTGCATCGCATTGTCTGAGTAGGTGTCATTC
TATTCTGGGGGGTGGGGTGGGGCAGGACAGCAAGGGGGAGGATTGGGAAGACAATAGCAGGC
ATGCTGGGGACACGTGCGGACCGAGCGGCCGCAGGAACCCCTAGTGATGGAGTTGGCCACTC
CCTCTCTGCGCGCTCGCTCGCTCACTGAGGCCGGGCGACCAAAGGTCGCCCGACGCCCGGGC
TTTGCCCGGGCGGCCTCAGTGAGCGAGCGAGCGCGCAGCTGCCTGCAGGGGCGCCTGATGCG
GTATTTTCTCCTTACGCATCTGTGCGGTATTTCACACCGCATACGTCAAAGCAACCATAGTA
CGCGCCCTGTAGCGGCGCATTAAGCGCGGCGGGTGTGGTGGTTACGCGCAGCGTGACCGCTA
CACTTGCCAGCGCCCTAGCGCCCGCTCCTTTCGCTTTCTTCCCTTCCTTTCTCGCCACGTTC
GCCGGCTTTCCCCGTCAAGCTCTAAATCGGGGGCTCCCTTTAGGGTTCCGATTTAGTGCTTT
ACGGCACCTCGACCCCAAAAAACTTGATTTGGGTGATGGTTCACGTAGTGGGCCATCGCCCT
GATAGACGGTTTTTCGCCCTTTGACGTTGGAGTCCACGTTCTTTAATAGTGGACTCTTGTTC
CAAACTGGAACAACACTCAACCCTATCTCGGGCTATTCTTTTGATTTATAAGGGATTTTGCC
GATTTCGGCCTATTGGTTAAAAAATGAGCTGATTTAACAAAAATTTAACGCGAATTTTAACA
AAATATTAACGTTTACAATTTTATGGTGCACTCTCAGTACAATCTGCTCTGATGCCGCATAG
TTAAGCCAGCCCCGACACCCGCCAACACCCGCTGACGCGCCCTGACGGGCTTGTCTGCTCCC
GGCATCCGCTTACAGACAAGCTGTGACCGTCTCCGGGAGCTGCATGTGTCAGAGGTTTTCAC
CGTCATCACCGAAACGCGCGAGACGAAAGGGCCTCGTGATACGCCTATTTTTATAGGTTAAT
GTCATGATAATAATGGTTTCTTAGACGTCAGGTGGCACTTTTCGGGGAAATGTGCGCGGAAC
CCCTATTTGTTTATTTTTCTAAATACATTCAAATATGTATCCGCTCATGAGACAATAACCCT
GATAAATGCTTCAATAATATTGAAAAAGGAAGAGTATGAGTATTCAACATTTCCGTGTCGCC
CTTATTCCCTTTTTTGCGGCATTTTGCCTTCCTGTTTTTGCTCACCCAGAAACGCTGGTGAA
AGTAAAAGATGCTGAAGATCAGTTGGGTGCACGAGTGGGTTACATCGAACTGGATCTCAACA
GCGGTAAGATCCTTGAGAGTTTTCGCCCCGAAGAACGTTTTCCAATGATGAGCACTTTTAAA
GTTCTGCTATGTGGCGCGGTATTATCCCGTATTGACGCCGGGCAAGAGCAACTCGGTCGCCG
CATACACTATTCTCAGAATGACTTGGTTGAGTACTCACCAGTCACAGAAAAGCATCTTACGG
ATGGCATGACAGTAAGAGAATTATGCAGTGCTGCCATAACCATGAGTGATAACACTGCGGCC
AACTTACTTCTGACAACGATCGGAGGACCGAAGGAGCTAACCGCTTTTTTGCACAACATGGG
GGATCATGTAACTCGCCTTGATCGTTGGGAACCGGAGCTGAATGAAGCCATACCAAACGACG
AGCGTGACACCA

In some embodiments, an AVV vector described herein comprises a nucleotide sequence at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 83. An exemplary nucleotide sequence for vector c.81.9 is set forth in SEQ ID NO: 83 (hGJB2 GRE9 underlined; mouse GJB2 coding sequence in bold face):

CGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCC
TGGCCTTTTGCTGGCCTTTTGCTCACATGTCCTGCAGGCAGCTGCGCGCTCGCTCGCTCACT
GAGGCCGCCCGGGCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCGGCCGCACGCGTT
CTAGGTAGACAACTAAGATGTTCATCTTATGGTTTAATGTTTAGTTGTAAAGGTTGTTTGCT
TCTCATTTGGTTCCAAGAAAGAGTATTTAGGCCAATTTCAGGGAGAAATATGTGTATAGATA
TATTCATATGTCAAACTGATTAGTGCTGAATGTCACATTTCCATATTCTAATAACATTTCTA
GCAAAGAAGAGGACACAGTGAAGAGAGAATTGCCCGCATTGTCATTGTCTCTTTCTGAGCCT
AGAACGCCTAACACTTGGGTGTGGAGAGACTCAGCCTCAATTCACTTTCTAGCAGCCACTGA
GATGTGCTTGCCTGGGGTGCCCCCTGGCAGGCAGGGCTGGAACTGCTTTCCAGTACCCACAC
GGACTGTGAACGAATCTTTCTTTGTGCTTTGTGTACAGAATGGAAGTTCAACAAATATTTGT
TGAATGTGTATGTCCTTCCAATACGCAGCAGCCCAGAGCAAACGTGGTAATCTTGTGTGTGT
TCATGTGAAAGCAGAATTTAATGGTGCTTTTAAGCACCAAAGTTTAAGATGCACGAGAAAAC
TGTATCTCCATTTTTTCCTTTTCGTTTACAATTACTTGTATAAGCCAGGCACGGTGGTGGCT
CACGCCTGTAATCCCAGCACTTTGGGAGGCCGAGGCGGGCGGATCACATGAGGTCGGGAGTT
AATTAAGACCTCGAAGGGGACTTGGGGGGTTCGGGGCTTTCGGGGGCGGTCGGGGGTTCGCG
GACCCGGGAAGCTCTGAGGACCCAGAGGCCGGGCGCGCTCCGCCCGCGGCGCCGCCCCCTCC
GTAACTTTCCCAGTCTCCGAGGGAAGAGGCGGGGTGTGGGGTGCGGTTAAAAGGCGCCACGG
CGGGAGACAGGTGTTGCGGCCCCGCAGCGCCCGCGCGCTCCTCTCCCCGACTCGGAGCCCCT
CGGCGGCGCCCGGCCCAGGACCCGCCTAGGAGCGCAGGAGCCCCAGCGCAGAGACCCCAACG
CCGAGACCCCCGCCCCGGCCCCGCCGCGCTTCCTCCCGACGCAGAGCAAACCGCCCAGAGTA
GAAGCCATGGATTGGGGCACACTCCAGAGCATCCTCGGGGGTGTCAACAAACACTCCACCAG
CATTGGAAAGATCTGGCTCACGGTCCTCTTCATCTTCCGCATCATGATCCTCGTGGTGGCTG
CAAAGGAGGTGTGGGGAGATGAGCAAGCCGATTTTGTCTGCAACACGCTCCAGCCTGGCTGC
AAGAATGTATGCTACGACCACCACTTCCCCATCTCTCACATCCGGCTCTGGGCTCTGCAGCT
GATCATGGTGTCCACGCCAGCCCTCCTGGTAGCTATGCATGTGGCCTACCGGAGACATGAAA
AGAAACGGAAGTTCATGAAGGGAGAGATAAAGAACGAGTTTAAGGACATCGAAGAGATCAAA
ACCCAGAAGGTCCGTATCGAAGGGTCCCTGTGGTGGACCTACACCACCAGCATCTTCTTCCG
GGTCATCTTTGAAGCCGTCTTCATGTACGTCTTTTACATCATGTACAATGGCTTCTTCATGC
AACGTCTGGTGAAATGCAACGCTTGGCCCTGCCCCAATACAGTGGACTGCTTCATTTCCAGG
CCCACAGAAAAGACTGTCTTCACCGTGTTTATGATTTCTGTGTCTGGAATTTGCATTCTGCT
AAATATCACAGAGCTGTGCTATTTGTTCGTTAGGTATTGCTCAGGAAAGTCCAAAAGACCAG
TCTACCCATACGATGTTCCAGATTACGCTTAAAGGCGCGCCACCCCTGCAGGGAATTCCGCA
TTGCCCAGTTGTTAGATTAAGAAATAGACAGCATGAGAGGGATGAGGCAACCCGTGCTCAGC
TGTCAAGGCTCAGTCGCTAGCATTTCCCAACACAAAGATTCTGACCTTAAATGCAACCATTT
GAAACCCCTGTAGGCCTCAGGTGAAACTCCAGATGCCACAATGGAGCTCTGCTCCCCTAAAG
CCTCAAAACAAAGGCCTAATTCTATGCCTGTCTTAATTTTCTTTCACTTAAGTTAGTTCCAC
TGAGACCCCAGGCTGTTAGGGGTTATTGGTGTAAGGTACTTTCATATTTTAAACAGAGGATA
TCGGCATTTGTTTCTTTCTCTGAGGACAAGAGAAAAAAGCCAGGTTCCACAGAGGACACAGA
GAAGGTTTGGGTGTCCTCCTGGGGTTCTTTTTGCCAACTTTCCCCACGTTAAAGGTGAACAT
TGGTTCTTTCATTTGCTTTGGAAGTTTTAATCTCTAACAGTGGACAAAGTTACCAGTGCCTT
AAACTCTGTTACACTTTTTGGAAGTGAAAACTTTGTAGTATGATAGGTTATTTTGATGTAAA
GATGTTCTGGATACCATTATATGTTCCCCCTGTTTCAGAGGCTCAGATTGTAATATGTAAAT
GGTATGTCATTCGCTACTATGATTTAATTTGAAATATGGTCTTTTGGTTATGAATACTTTGC
AGCACAGCTGAGAGGCTGTCTGTTGTATTCATTGTGGTCATAGCACCTAACAACATTGTAGC
CTCAATCGAGTGAGACAGACTAGAAGTTCCTAGTGATGGCTTATGATAGCAAATGGCCTCAT
GTCAAATATTTAGATGTAATTTTGTGTAAGAAATACAGACTGGATGTACCACCAACTACTAC
CTGTAATGACAGGCCTGTCCAACACATCTCCCTTTTCCATGACTGTGGTAGCCAGCATCGGA
AAGAACGCTGATTTAAAGAGGTCGCTTGGGAATTTTATTGACACAGTACCATTTAATGGGGA
GGACAAAATGGGGCAGGGGAGGGAGAAGTTTCTGTCGTTAAAAACAGATTTGGAAAGACTGG
ACTCTAAAGTCTGTTGATTAAAGATGAGCTTTGTCTACTTCAAAAGTTTGTTTGCTTACCCC
TTCAGCCTCCAATTTTTTAAGTGAAAATATAGCTAATAACATGTGAAAAGAATAGAAGCTAA
GGTTTAGATAAATATTGAGCAGATCTATAGGAAGATTGAACCTGAATATTGCCATTATGCTT
GACATGGTTTCCAAAAAATGGTACTCCACATATTTCAGTGAGGGTAAGTATTTTCCTGTTGT
CAAGAATAGCATTGTAAAAGCATTTTGTAATAATAAAGAATAGCTTTAATGATATGCTTGTA
ACTAAAATAATTTTGTAATGTATCAAATACATTTAAAACATTAAAATATAATCTCTATAATA
ATTTAAAATCTAATATGGTTTTAATAGAACAGCGATATCAAGCTTATCGATAATCAACCTCT
GGATTACAAAATTTGTGAAAGATTGACTGGTATTCTTAACTATGTTGCTCCTTTTACGCTAT
GTGGATACGCTGCTTTAATGCCTTTGTATCATGCTATTGCTTCCCGTATGGCTTTCATTTTC
TCCTCCTTGTATAAATCCTGGTTGCTGTCTCTTTATGAGGAGTTGTGGCCCGTTGTCAGGCA
ACGTGGCGTGGTGTGCACTGTGTTTGCTGACGCAACCCCCACTGGTTGGGGCATTGCCACCA
CCTGTCAGCTCCTTTCCGGGACTTTCGCTTTCCCCCTCCCTATTGCCACGGCGGAACTCATC
GCCGCCTGCCTTGCCCGCTGCTGGACAGGGGCTCGGCTGTTGGGCACTGACAATTCCGTGGT
GTTGTCGGGGAAATCATCGTCCTTTCCTTGGCTGCTCGCCTATGTTGCCACCTGGATTCTGC
GCGGGACGTCCTTCTGCTACGTCCCTTCGGCCCTCAATCCAGCGGACCTTCCTTCCCGCGGC
CTGCTGCCGGCTCTGCGGCCTCTTCCGCGTCTTCGCCTTCGCCCTCAGACGAGTCGGATCTC
CCTTTGGGCCGCCTCCCCGCGAATTCATCGATACCGAGCGCTGCTCGAGAGATCTGTGATAG
CGGCCATCAAGCTGGCTGTGCCTTCTAGTTGCCAGCCATCTGTTGTTTGCCCCTCCCCCGTG
CCTTCCTTGACCCTGGAAGGTGCCACTCCCACTGTCCTTTCCTAATAAAATGAGGAAATTGC
ATCGCATTGTCTGAGTAGGTGTCATTCTATTCTGGGGGGTGGGGTGGGGCAGGACAGCAAGG
GGGAGGATTGGGAAGACAATAGCAGGCATGCTGGGGACACGTGCGGACCGAGCGGCCGCAGG
AACCCCTAGTGATGGAGTTGGCCACTCCCTCTCTGCGCGCTCGCTCGCTCACTGAGGCCGGG
CGACCAAAGGTCGCCCGACGCCCGGGCTTTGCCCGGGCGGCCTCAGTGAGCGAGCGAGCGCG
CAGCTGCCTGCAGGGGCGCCTGATGCGGTATTTTCTCCTTACGCATCTGTGCGGTATTTCAC
ACCGCATACGTCAAAGCAACCATAGTACGCGCCCTGTAGCGGCGCATTAAGCGCGGCGGGTG
TGGTGGTTACGCGCAGCGTGACCGCTACACTTGCCAGCGCCCTAGCGCCCGCTCCTTTCGCT
TTCTTCCCTTCCTTTCTCGCCACGTTCGCCGGCTTTCCCCGTCAAGCTCTAAATCGGGGGCT
CCCTTTAGGGTTCCGATTTAGTGCTTTACGGCACCTCGACCCCAAAAAACTTGATTTGGGTG
ATGGTTCACGTAGTGGGCCATCGCCCTGATAGACGGTTTTTCGCCCTTTGACGTTGGAGTCC
ACGTTCTTTAATAGTGGACTCTTGTTCCAAACTGGAACAACACTCAACCCTATCTCGGGCTA
TTCTTTTGATTTATAAGGGATTTTGCCGATTTCGGCCTATTGGTTAAAAAATGAGCTGATTT
AACAAAAATTTAACGCGAATTTTAACAAAATATTAACGTTTACAATTTTATGGTGCACTCTC
AGTACAATCTGCTCTGATGCCGCATAGTTAAGCCAGCCCCGACACCCGCCAACACCCGCTGA
CGCGCCCTGACGGGCTTGTCTGCTCCCGGCATCCGCTTACAGACAAGCTGTGACCGTCTCCG
GGAGCTGCATGTGTCAGAGGTTTTCACCGTCATCACCGAAACGCGCGAGACGAAAGGGCCTC
GTGATACGCCTATTTTTATAGGTTAATGTCATGATAATAATGGTTTCTTAGACGTCAGGTGG
CACTTTTCGGGGAAATGTGCGCGGAACCCCTATTTGTTTATTTTTCTAAATACATTCAAATA
TGTATCCGCTCATGAGACAATAACCCTGATAAATGCTTCAATAATATTGAAAAAGGAAGAGT
ATGAGTATTCAACATTTCCGTGTCGCCCTTATTCCCTTTTTTGCGGCATTTTGCCTTCCTGT
TTTTGCTCACCCAGAAACGCTGGTGAAAGTAAAAGATGCTGAAGATCAGTTGGGTGCACGAG
TGGGTTACATCGAACTGGATCTCAACAGCGGTAAGATCCTTGAGAGTTTTCGCCCCGAAGAA
CGTTTTCCAATGATGAGCACTTTTAAAGTTCTGCTATGTGGCGCGGTATTATCCCGTATTGA
CGCCGGGCAAGAGCAACTCGGTCGCCGCATACACTATTCTCAGAATGACTTGGTTGAGTACT
CACCAGTCACAGAAAAGCATCTTACGGATGGCATGACAGTAAGAGAATTATGCAGTGCTGCC
ATAACCATGAGTGATAACACTGCGGCCAACTTACTTCTGACAACGATCGGAGGACCGAAGGA
GCTAACCGCTTTTTTGCACAACATGGGGGATCATGTAACTCGCCTTGATCGTTGGGAACCGG
AGCTGAATGAAGCCATACCAAACGACGAGCGTGACACCA

II. Recombinant Adeno-Associated Viruses (rAAVs)

In some aspects, the disclosure provides isolated AAVs. As used herein with respect to AAVs, the term “isolated” refers to an AAV that has been artificially produced, engineered, or obtained. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s). The AAV capsid is an important element in determining these tissue-specific targeting capabilities. Thus, a rAAV having a capsid appropriate for the tissue being targeted can be selected.

Methods for obtaining recombinant AAVs having a desired capsid protein are known in the art. (See, for example, US 2003/0138772, which is incorporated herein by reference). Typically the methods involve culturing a host cell which contains a nucleic acid sequence encoding an AAV capsid protein; a functional rep gene; a recombinant AAV vector composed of AAV inverted terminal repeats (ITRs) and an expression cassette (e.g., GJB2 expression cassette); and a helper plasmid expressing the E2b and E4 transcripts from adenovirus to permit packaging of the recombinant AAV vector into the AAV capsid. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa, and about 62 kDa. In some embodiments, upon translation, capsid proteins form a spherical 60-mer protein shell around the viral genome. In some embodiments, the functions of the capsid proteins are to protect the viral genome, deliver the genome, and/or interact with the host. In some aspects, capsid proteins deliver the viral genome to a host in a tissue specific manner (e.g., to cells in the inner ear).

The present disclosure is based in part on the finding that certain AAV serotype capsids are capable of delivering a transgene (e.g., GJB2 gene) to the ear (e.g., cells in the inner ear). In some embodiments, an AAV capsid protein is of an AAV serotype selected from the group consisting of AAV9.PHP.B, AAV9.PHP.eB, exoAAV, Anc80, AAV1, AAV2, AAV3, AAV4, AAV5, AAV6, AAV7, AAV8, AAVrh8, AAV9, AAV10, AAVrh10, and AAV-S. AAV2.7m8 is capable of delivering a transgene targeting cochlear hair cells and supporting cells and the retina. AAV2.7m8 shows good transduction to the inner ear (Isgrig et al., “AAV2.7m8 is a powerful viral vector for inner ear gene therapy,” Nature Communications volume 10, Article number: 427 (2019)). In some embodiments, the capsid protein is of AAV serotype 9 (AAV9). In some embodiments, an AAV capsid protein is of a serotype derived from AAV9 (e.g., an AAV9 capsid variant), for example, AAV9.PHP.B. In some embodiments, the AAV9 capsid variant is AAV9.PHP.B. In some embodiments, the AAV9 capsid variant is AAV-S. AAV-S is an AAV9 capsid protein variant originally developed for targeting central nervous system (CNS) (Hanlon et al, Selection of an Efficient AAV Vector for Robust CNS Transgene Expression, Molecular Therapy Method & Clinical Development, vol. 15, pp. 320-332, Dec. 13, 2019, and PCT/US2020/025720, which are incorporated herein by reference). Surprisingly, AAV-S showed good transducing efficiency for inner ear cells, (see., e,g., Hanlon et al., AAV-S: A novel AAV vector selected in brain transduces the inner ear with high efficiency, Molecular Therapy Vol 18 No 4S1, Apr. 28, 2020, Abstract 151, which is incorporated herein by reference), including, but not limited to: outer hair cells (OHCs), inner hair cells (IHCs), supporting cells (e.g., border cell, inner phalangeal cell, inner pillar cell, outer pillar cell, Deiters' cell, Hensen's, or Claudius' cell), spiral ganglion neuron, spiral limbus cells (e.g., glial cell or interdental cell), outer sulcus cells, lateral wall, stria vascularis (e.g., basal cell and intermediate cell), inner sulcus, spiral ligament (e.g., fibrocytes), or cells of the vestibular system. In some embodiments, the AAV capsid is AAV-S. An exemplary amino acid sequence for AAV-S is set forth in SEQ ID NO: 33. In some embodiments, the AAV capsid is an exoAAV. An exoAAV refers to an exosome-associated AAV. An exoAAV capsid protein may be selected from the group consisting of AAV1, AAV2, AAV3, AAV4, AAV5, AAV6, AAV7, AAV8, AAVrh8, AAV9, AAV10, AAVrh10, and AAV.PHP.B. In some examples, the exoAAV is exoAAV1 or exoAAV9.

Exemplary amino acid sequence for AAV-S is set forth in SEQ ID NO: 33:

MAADGYLPDWLEDNLSEGIREWWALKPGAPQPKANQQHQDNARGLVLPG
YKYLGPGNGLDKGEPVNAADAAALEHDKAYDQQLKAGDNPYLKYNHADA
EFQERLKEDTSFGGNLGRAVFQAKKRLLEPLGLVEEAAKTAPGKKRPVE
QSPQEPDSSAGIGKSGAQPAKKRLNFGQTGDTESVPDPQPIGEPPAAPS
GVGSLTMASGGGAPVADNNEGADGVGSSSGNWHCDSQWLGDRVITTSTR
TWALPTYNNHLYKQISNSTSGGSSNDNAYFGYSTPWGYFDFNRFHCHFS
PRDWQRLINNNWGFRPKRLNFKLFNIQVKEVTDNNGVKTIANNLTSTVQ
VFTDSDYQLPYVLGSAHEGCLPPFPADVFMIPQYGYLTLNDGSQAVGRS
SFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVS
TTVTQNNNSEFAWPGASSWALNGRNSLMNPGPAMASHKEGEDRFFPLSG
SLIFGKQGTGRDNVDADKVMITNEEEIKTTNPVATESYGQVATNHQSAQ
STTLYSPAQAQTGWVQNQGILPGMVWQDRDVYLQGPIWAKIPHTDGNFH
PSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIG
TRYLTRNL

The skilled artisan will also realize that conservative amino acid substitutions may be made to provide functionally equivalent variants or homologs of the capsid proteins. In some aspects, the disclosure embraces sequence alterations that result in conservative amino acid substitutions. As used herein, a conservative amino acid substitution refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made among amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the proteins and polypeptides described herein (e.g., GJB2 protein sequence).

In some embodiments, the rAAV is a single stranded AAV (ssAAV). A ssAAV, as used herein, refers to a rAAV with the coding sequence and complementary sequence of the transgene expression cassette on separate strands and packaged in separate viral capsids. In some embodiments, the rAAV is a self-complementary AAV (scAAV). A scAAV, as used herein, refers to a rAAV with both the coding and complementary sequence of the transgene expression cassette present on the single strand of an AAV genome. The coding region of a scAAV was designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription.

In some embodiments, the rAAV as provided herein, is capable of delivering the transgene (e.g., GJB2) to a mammal. In some examples, the mammal can be a human or a non-human mammal, such as a mouse, a rat, or a non-human primate (e.g., cynomolgus monkey), a cat, a dog, a pig, a horse, a donkey, a camel, a sheep, or a goat. In certain embodiments, the mammal is a human.

In some embodiments, the rAAV, as provided herein, is capable of delivering the transgene (e.g., GJB2) to the ear. In some instances, the rAAV. as provided herein, is capable of delivering the transgene (e.g., GJB2) to the cells in the inner ear (e.g., cochlea, saccule, utricle and semicircular canals). Non-limiting examples of the target cells are outer hair cells (OHC), inner hair cells (IHC), spiral ganglion neurons, cells of stria vascularis, cells of inner sulcus, cells of spiral ligament, cells of vestibular system, organ of Corti supporting cells (e,g., epithelial cells of the inner and outer sulcus, and interdental cells), interdental cells in the spiral limbus, root cells within the spiral ligament, pillar cells, Deiters' cells, Hensen's cells, Claudius cells, inner phalangeal cells; and border cells, strial intermediate cells, fibrocytes of the lateral wall and suprastrial zone, basal cells of the stria vascularis, fibrocytes in the spiral ligament, fibrocytes in the spiral limbus, mesenchymal cells lining the bony otic capsule facing the scala vestibuli, and supralimbal dark cells. In some embodiments, the combination of an AAV capsid having tropism to the inner ear (e.g., AAV-S or AAV-PHP.B) and the isolated nucleic acid described herein (e.g., an isolated nucleic acid driving GJB2 expression under the control of GJB2 gene regulatory elements) is superior in GJB2 gene replacement therapy to that it limits GJB2 expression to cells that normally express it, and reduces toxicity associated with promiscuous GJB2 expression (e.g., toxicity associated with GJB2 being expressed in hair cells and/or the central nervous system (CNS)).

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the instant disclosure relates to a host cell containing a nucleic acid that comprises a coding sequence encoding a protein (e.g., GJB2 protein). In some embodiments, the host cell is a mammalian cell (e.g., a human cell), a yeast cell, a bacterial cell, an insect cell, a plant cell, or a fungal cell.

The recombinant AAV vector, rep sequences, cap sequences, and helper functions required for producing the rAAV of the disclosure may be delivered to the packaging host cell using any appropriate genetic element (e.g., vector). The selected genetic element may be delivered by any suitable method, including those described herein and known in the art. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are known in the art, and the selection of a suitable method is not a limitation on the present disclosure. See, e.g., K. Fisher et al., J. Virol., 70:520-532 (1993) and U.S. Pat. No. 5,478,745, each of which is incorporated herein by reference.

In some embodiments, recombinant AAVs may be produced using the triple transfection method (described in detail in U.S. Pat. No. 6,001,650, which is incorporated herein by reference). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, both of which are incorporated herein by reference. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses, such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a Laboratory Manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids into suitable host cells.

A “host cell” refers to any cell that harbors, or is capable of harboring, a substance of interest. Often a host cell is a mammalian cell. A host cell may be used as a recipient of an AAV helper construct, an AAV plasmid, an accessory function vector, or other transfer DNA associated with the production of recombinant AAVs. The term includes the progeny of the original cell which has been transfected. Thus, a “host cell,” as used herein, may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation or engineering.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the term “recombinant cell” refers to a cell into which an exogenous DNA segment, such as DNA segment that leads to the transcription of a biologically-active polypeptide (e.g., GJB2 protein), has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. The term “expression vector or construct” means any type of genetic construct containing a nucleic acid in which part or all of the nucleic acid encoding sequence is capable of being transcribed.

The foregoing methods for packaging recombinant vectors in desired AAV capsids to produce the rAAVs of the disclosure are not meant to be limiting and other suitable methods will be apparent to the skilled artisan.

The present disclosure, provides a rAAV comprising a vector (e.g., AAV vectors) for expressing a transgene (e.g., GJB2), such vectors include AAV LTRs (e.g., AAV2 LTRs) and an expression cassette comprising a promoter operably linked to a promoter (e.g., human GJB2 promoter or fragment thereof). In addition, the vector can further comprise certain regulatory elements (e.g., GJB2 enhancers, 5′ and 3′ UTRs of the GJB2 gene, WPRE, and poly adenylation sites). In addition, the rAAV can comprise a capsid protein (e.g., AAV9.PHP.B capsid or AAV-S capsid). Such rAAV can deliver transgenes (e.g., GJB2) to target tissues (e.g., cells that normally express GJB2 in the inner ear). In some embodiments, such a rAAV is capable of delivering transgenes (e.g., GJB2) into specific cells in the target tissue, for example, connective tissue cells of the cochlea and supporting cells of the organ of Corti and nearby regions.

### III. Pharmaceutical Composition

The rAAVs may be delivered to a subject in compositions according to any appropriate method known in the art. The rAAV, preferably suspended in a physiologically compatible carrier (i.e., in a composition), may be administered to a subject, e.g., host animal, patient, experimental animal. In some embodiments, the subject is a mammal. In some examples, the mammal is a human. In other embodiments, the mammal can be a non-human mammal, such as a human, mouse, rat, cat, dog, sheep, rabbit, horse, cow, goat, pig, guinea pig, hamster, chicken, turkey, or a non-human primate (e.g., cynomolgus monkey). The subject may be at any stage of development and of any gender.

The rAAV can be delivered to any organ or tissue of interest. In some embodiments, the rAAV is delivered to the inner ear. Delivery of the rAAVs to a mammalian subject may be by, for example, injection to the ear. In some embodiments, the injection is to the ear through the round window membrane of the inner ear, into the scala media of the cochlea, into the scala vestibuli of the cochlea, into a semicircular canal of the inner ear, or into the saccule or the utricle of the inner ear. In some embodiments, the rAAV is delivered to the ear by topical administration (e.g., ear drops). In some embodiments, the injection is not topical administration. Combinations of administration methods (e.g., topical administration and injection through round window membrane of the inner ear) can also be used.

The compositions of the disclosure may comprise a rAAV described herein alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

In some embodiments, a composition further comprises a pharmaceutically acceptable carrier. Suitable carriers may be readily selected by one of skill in the art in view of the indication for which the rAAV is directed. “Acceptable” means that the carrier must be compatible with the rAAV or the isolated nucleic acid of the composition (and preferably, capable of stabilizing the active ingredient) and not deleterious to the subject to be treated. In some embodiments, the pharmaceutically acceptable carrier/excipient is compatible with the mode of administration. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover. For example, one acceptable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

The rAAV containing pharmaceutical composition disclosed herein may further comprise a suitable buffer agent. A buffer agent is a weak acid or base used to maintain the pH of a solution near a chosen value after the addition of another acid or base. In some examples, the buffer agent disclosed herein can be a buffer agent capable of maintaining physiological pH despite changes in carbon dioxide concentration (e.g., produced by cellular respiration). Exemplary buffer agents include, but are not limited to, HEPES (4-(2-hydroxyethyl)-1-piperazineethanesulfonic acid) buffer, Dulbecco's phosphate-buffered saline (DPBS) buffer, or phosphate-buffered saline (PBS) buffer. Such buffers may comprise disodium hydrogen phosphate and sodium chloride, or potassium dihydrogen phosphate and potassium chloride.

Optionally, the compositions of the disclosure may contain, in addition to the rAAV and carrier(s), other pharmaceutical ingredients, such as preservatives or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The rAAV containing pharmaceutical composition described herein comprises one or more suitable surface-active agents, such as a surfactant. Surfactants are compounds that lower the surface tension (or interfacial tension) between two liquids, between a gas and a liquid, or between a liquid and a solid. Surfactants may act as detergents, wetting agents, emulsifiers, foaming agents, and dispersants. Suitable surfactants include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface active agent will conveniently comprise between 0.05 and 5% surface-active agent, and can be between 0.1 and 2.5%. It will be appreciated that other ingredients may be added, for example, mannitol or other pharmaceutically acceptable vehicles, if necessary.

The rAAVs are administered in sufficient amounts to transfect the cells of a desired tissue (e.g., connective tissue cells of the cochlea and supporting cells of the organ of Corti and nearby regions) and to provide sufficient levels of gene transfer and expression without undue adverse effects. Examples of pharmaceutically acceptable routes of administration include, but are not limited to, direct delivery to the selected organ (e.g., the ear) or tissue, intravenous, intramuscular, subcutaneous, intradermal, intratumoral, and other parental routes of administration. Routes of administration may be combined, if desired.

The dose of rAAV virions required to achieve a particular “therapeutic effect,” e.g., the units of dose in viral genome copies per kilogram of body weight (GC/kg or VG/kg), will vary based on several factors including, but not limited to: the route of rAAV virion administration, the level of gene or RNA expression required to achieve a therapeutic effect, the specific disease or disorder being treated, and the stability of the gene or rAAV product. One of skill in the art can readily determine a rAAV virion dose range to treat a patient having a particular disease or disorder (e.g., nonsyndromic hearing loss and deafness, or any GJB2-associated disorders) based on the aforementioned factors, as well as other factors.

An effective amount of a rAAV is an amount sufficient to infect an animal (e.g., mouse, rat, non-human primate or human) or target a desired tissue or cell (e.g., connective tissue cells of the cochlea and supporting cells of the organ of Corti and nearby regions). The effective amount will depend primarily on factors, such as the species, age, weight, health of the subject, and the tissue to be targeted, and may thus vary among animals and tissue. For example, an effective amount of the rAAV is generally in the range of from about 1 ml to about 100 ml of solution containing from about 10⁹ to 10¹⁶ genome copies. In some cases, a dosage between about 10¹¹ to 10¹³ rAAV genome copies is appropriate. In certain embodiments, 10⁹ rAAV genome copies are effective to target inner ear tissue (e.g., connective tissue cells of the cochlea and supporting cells of the organ of Corti and nearby regions). In some embodiments, a dose more concentrated than 10⁹ rAAV genome copies is toxic when administered to the ear of a subject. In some embodiments, an effective amount is produced by multiple doses of a rAAV.

In some embodiments, a dose of rAAV is administered to a subject no more than once per day (e.g., a 24-hour period). In some embodiments, a dose of rAAV is administered to a subject no more than once per 2, 3, 4, 5, 6, or 7 days. In some embodiments, a dose of rAAV is administered to a subject no more than once per week (e.g., 7 calendar days). In some embodiments, a dose of rAAV is administered to a subject no more than bi-weekly (e.g., once in a two-week period). In some embodiments, a dose of rAAV is administered to a subject no more than once per month (e.g., once in 30 calendar days). In some embodiments, a dose of rAAV is administered to a subject no more than once per six months. In some embodiments, a dose of rAAV is administered to a subject no more than once per year (e.g., 365 days or 366 days in a leap year). In some embodiments, a dose of rAAV is administered to a subject once in a lifetime.

In some embodiments, rAAV compositions are formulated to reduce aggregation of AAV particles in the composition, particularly where high rAAV concentrations are present (e.g., ˜10¹³ GC/ml or more). Appropriate methods for reducing aggregation may be used, including, for example, addition of surfactants, pH adjustment, salt concentration adjustment, etc. (See, e.g., Wright et al., Molecular Therapy (2005) 12, 171-178, the contents of which are incorporated herein by reference.)

Formulation of pharmaceutically acceptable excipients and carrier solutions is well known to those of skill in the art, as is the development of suitable dosing and treatment regimens for using the particular compositions described herein in a variety of treatment regimens. Factors, such as solubility, bioavailability, biological half-life, route of administration, product shelf life, as well as other pharmacological considerations, will be contemplated by one skilled in the art of preparing such pharmaceutical formulations, and as such, a variety of dosages and treatment regimens may be desirable.

In some embodiments, rAAVs in suitably formulated pharmaceutical compositions disclosed herein are delivered directly to target tissue, e.g., direct to inner ear tissue (e.g., connective tissue cells of the cochlea and supporting cells of the organ of Corti and nearby regions). However, in certain circumstances it may be desirable to separately or in addition deliver the rAAV-based therapeutic constructs via another route, e.g., subcutaneously, parenterally, intravenously, intramuscularly, intrathecally, orally, or intraperitoneally. In some embodiments, the administration modalities as described in U.S. Pat. Nos. 5,543,158; 5,641,515 and 5,399,363 (each specifically incorporated herein by reference in its entirety) may be used to deliver rAAVs.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases, the form is sterile. It must be stable under the conditions of manufacture and storage and must be preserved to prevent contamination with microorganisms, such as bacteria, fungi, and other viruses. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of contamination by microorganisms can be achieved by various antibacterial and antifungal agents, for example, parabens, chlorobutanol, phenol, sorbic acid, thimerosal, and the like. In many cases, it will be preferable to include isotonic agents, for example, sugars or salts (e.g., sodium chloride). Prolonged absorption of the injectable composition can be achieved by the use in the composition of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous administration, intramuscular administration, subcutaneous administration, intraperitoneal administration, and injection through the round window membrane of the inner ear. In this respect, a suitable sterile aqueous medium may be employed. For example, one dosage may be dissolved in 1 ml of isotonic NaCl solution and either added to 1000 ml of hypodermoclysis fluid or injected at the proposed site of infusion (see for example, Remington's Pharmaceutical Sciences 15th Edition, pages 1035-1038 and 1570-1580). Some variation in dosage will necessarily occur depending on the condition of the host. The person responsible for administration will, in any event, determine the appropriate dose for the individual subject/host.

Sterile injectable solutions are prepared by incorporating the active rAAV in the required amount in the appropriate solvent with various of the other ingredients described herein, as required, followed by filter sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts include but are not limited to hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, solvents, coatings, diluents, antibacterial and antifungal agents, isotonic and absorption delaying agents, buffers, carrier solutions, suspensions, colloids, and the like. The use of such media and agents for pharmaceutically active substances is well known in the art. Supplemental active ingredients can also be incorporated into the compositions. The phrase “pharmaceutically acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, a nanoparticle, or the like.

Such formulations may be preferred for the introduction of pharmaceutically acceptable formulations of the nucleic acids or the rAAV constructs disclosed herein. The formation and use of liposomes are generally known to those of skill in the art. Recently, liposomes were developed with improved serum stability and circulation half-times (U.S. Pat. No. 5,741,516, which is incorporated herein by reference). Further, various methods of liposome and liposome-like preparations as potential drug carriers have been described (U.S. Pat. Nos. 5,567,434; 5,552,157; 5,565,213; 5,738,868 and 5,795,587, each of which is incorporated herein by reference).

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

### IV. Therapeutic Applications

The present disclosure also provides methods for delivering (e.g., by an isolated nucleic acid, a vector, a rAAV, a host cell, or a pharmaceutical composition described herein) a transgene (e.g., GJB2) to cells that normally express the transgene (e.g., GJB2) in the ear (e.g., connective tissue cells of the cochlea and supporting cells of the organ of Corti and nearby regions) of a subject for treating hearing loss. In some aspects, the present disclosure provides a method for treating GJB2 associated diseases (e.g., non-syndromic Hearing Loss and Deafness (DFNB1)) in a subject by delivering (e.g., by an isolated nucleic acid, a vector, a rAAV, a host cell, or a pharmaceutical composition described herein) a transgene (e.g., GJB2) to cells that normally express the transgene (e.g., GJB2) in the ear (e.g., connective tissue cells of the cochlea and supporting cells of the organ of Corti and nearby regions) of a subject. In some aspects, the present disclosure provides a method for targeted GJB2 expression in inner ear supporting cells and/or detargeting GJB2 in neuron and/or cochlear hair cells by delivering (e.g., by an isolated nucleic acid, a vector, a rAAV, a host cell, or a pharmaceutical composition described herein) a transgene (e.g., GJB2) to cells that normally express the transgene (e.g., GJB2) in the ear (e.g., connective tissue cells of the cochlea and supporting cells of the organ of Corti and nearby regions) of a subject. In some embodiments, the targeted GJB2 expression in inner ear supporting cells and/or detargeting GJB2 in neuron and/or cochlear hair cells is designed to treat GJB2 associated diseases described herein. In some embodiments, the subject is a mammal. In some examples, the subject is a human. In other embodiments, the subject is a non-human mammal, such as a mouse, rat, cow, goat, pig, camel, or non-human primate (e.g., cynomolgus monkey).

In some embodiments, the subject is having or suspected of having hearing loss. In certain embodiments, the subject is diagnosed with having non-syndromic Hearing Loss and Deafness (DFNB1). In certain embodiments, the hearing loss is associated with a mutation in the GJB2 gene. In some embodiments, the mutation of GJB2 gene is a point mutation, a missense mutation, a nonsense mutation, a deletion, an insertion, or a combination thereof. Non-limiting examples of mutations in the GJB2 gene are shown in Table 2. A mutation, as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue.

TABLE 2
Exemplary mutations in GJB2 gene (Nucleotide number starting at the ATG of NM_004004.6).

| Mutation | Amino Acid Change | Mutation | Amino Acid Change |
|---|---|---|---|
| c.677T > G | p.Val226Gly | c.509_522del | p.Asn170ThrfsTer35 |
| c.677T > A | p.Val226Asp | c.521G > C | p.Cys174Ser |
| c.674C > T | p.Pro225Leu | c.520T > C | p.Cys174Arg |
| c.653G > A | p.Cys218Tyr | c.518C > G | p.Pro173Arg |
| c.647_650del | p.Arg216IlefsTer17 | c.517C > T | p.Pro173Ser |
| c.650A > G | p.Tyr217Cys | c.516G > C | p.Trp172Cys |
| c.645del | p.Arg216AspfsTer18 | c.516G > A | p.Trp172Ter |
| c.641T > C | p.Leu214Pro | c.514T > A | p.Trp172Arg |
| c.638T > A | p.Leu213Ter | c.512_513insAACG | p.Trp172ThrfsTer39 |
| c.632_633del | p.Cys211LeufsTer5 | c.508_511dup | p.Ala171GlufsTer40 |
| c.633T > A | p.Cys211Ter | c.511G > T | p.Ala171Ser |
| c.632G > A | p.Cys211Tyr | c.509dup | p.Asn170LysfsTer40 |
| c.622A > C | p.Thr208Pro | c.509A > C | p.Asn170Thr |
| c.617A > G | p.Asn206Ser | c.506G > A | p.Cys169Tyr |
| c.617A > C | p.Asn206Thr | c.504_505insAAGG | p.Cys169LysfsTer42 |
| c.614T > C | p.Leu205Pro | c.505T > C | p.Cys169Arg |
| c.613C > G | p.Leu205Val | c.488T > C | p.Met163Thr |
| c.608_609delinsAA | p.Ile203Lys | c.487A > G | p.Met163Val |
| c.605G > T | p.Cys202Phe | c.487A > C | p.Met163Leu |
| c.592_600delinsCAGTGTTCATGACATTC | p.Val198GlnfsTer4 | c.486_487insT | p.Met163TyrfsTer47 |
| c.599G > C | p.Gly200Ala | c.482T > C; | p.Phe161Ser |
| c.598G > T | p.Gly200Ter | c.476A > T | p.Asp159Val |
| c.598G > A | p.Gly200Arg | c.458_475dup | p.Val153_Tyr158dup |
| c.596C > T | p.Ser199Phe | c.475G > A | p.Asp159Asn |
| c.592G > A | p.Val198Met | c.473A > G | p.Tyr158Cys |
| c.589G > T | p.Ala197Ser | c.464_465del | p.Tyr155CysfsTer54 |
| c.585G > C | p.Met195Ile | c.465T > A | p.Tyr155Ter |
| c.584T > C | p.Met195Thr | c.458T > C | p.Val153Ala |
| c.583A > G | p.Met195Val | c.456C > A | p.Tyr152Ter |
| c.576del | p.Val193CysfsTer3 | c.452T > G | p.Met151Arg |
| c.575_576del | p.Thr192SerfsTer17 | c.431_450del | p.Val144AspfsTer59 |
| c.585G > C | p.Met195Ile | c.439G > T | p.Glu147Ter |
| c.584T > C | p.Met195Thr | c.439G > A | p.Glu147Lys |
| c.583A > G | p.Met195Val | c.435_436del | p.Phe146ArgfsTer63 |
| c.576del | p.Val193CysfsTer3 | c.428G > T | p.Arg143Leu |
| c.575_576del | p.Thr192SerfsTer17 | c.428G > A | p.Arg143Gln |
| c.572del | p.Phe191SerfsTer5 | c.427C > T | p.Arg143Trp |
| c.569T > A | p.Val190Asp | c.424_426del | p.Phe142del |
| c.564_565del | p.Lys188AsnfsTer21 | c.426C > G | p.Phe142Leu |
| c.563A > G | p.Lys188Arg | c.426C > A | p.Phe142Leu |
| c.559_561del | p.Glu187del | c.424T > C | p.Phe142Leu |
| c.557C > T | p.Thr186Met | c.424T > A | p.Phe142Ile |
| c.557C > A | p.Thr186Lys | c.419T > G | p.Ile140Ser |
| c.551G > C | p.Arg184Pro | c.416G > A | p.Ser139Asn |
| c.551G > A | p.Arg184Gln | c.415A > T | p.Ser139Cys |
| c.550C > T | p.Arg184Trp | c.413G > A | p.Ser138Asn |
| c.550C > G | p.Arg184Gly | c.409dup | p.Thr137AsnfsTer73 |
| c.548C > T | p.Ser183Phe | c.408C > A | p.Tyr136Ter |
| c.535G > C | p.Asp179His | c.407A > G | p.Tyr136Cys |
| c.523C > A | p.Pro175Thr | c.405del | p.Tyr136ThrfsTer32 |
| c.535G > A | p.Asp179Asn | c.402del | p.Trp134Ter |
| c.533T > C | p.Val178Ala | c.401G > A | p.Trp134Ter |
| c.516_532del | p.Trp172CysfsTer32 | c.400T > C | p.Trp134Arg |
| c.390_399del | p.Ser131GlyfsTer34 | c.389G > A | p.Gly130Asp |
| c.398G > A | p.Trp133Ter | c.384C > G | p.Ile128Met |
| c.397T > G | p.Trp133Gly | c.377_383dup | p.Glu129ProfsTer83 |
| c.394C > G | p.Leu132Val | c.382A > G | p.Ile128Val |
| c.389G > T | p.Gly130Val | c.380G > T | p.Arg127Leu |
| c.389G > C | p.Gly130Ala | c.379C > T | p.Arg127Cys |
| c.377_378insATGCGGA | p.Arg127CysfsTer85 | c.344T > G | p.Phe115Cys |
| c.370C > T | p.Gln124Ter | c.340G > T | p.Glu114Ter |
| c.367del | p.Thr123ProfsTer45 | c.339T > G | p.Ser113Arg |
| c.365A > T | p.Lys122Ile | c.336G > T; | p.Lys112Asn |
| c.363del | p.Thr123ProfsTer45 | c.334_335del | p.Lys112GlufsTer2 |
| c.355_363del | p.Glu119_Ile121del | c.335A > T | p.Lys112Met |
| c.358_360del | p.Glu120del | c.331A > G | p.Ile111Val |
| c.358G > A | p.Glu120Lys | c.329del | p.Glu110GlyfsTer2 |
| c.355G > A | p.Glu119Lys | c.328del | p.Glu110ArgfsTer2 |
| c.345dup | p.Lys116Ter | c.327_328del | p.Glu110AspfsTer4 |
| c.328G > A | p.Glu110Lys | c.299_300del | p.His100ArgfsTer14 |
| c.327_328delinsA | p.Glu110ArgfsTer2 | c.300T > A | p.His100Gln |
| c.314_327del | p.Lys105ArgfsTer5 | c.299A > T | p.His100Leu |
| c.313_326del | p.Lys105GlyfsTer5 | c.299A > C | p.His100Pro |
| c.326G > T | p.Gly109Val | c.292_298dup | p.His100ProfsTer4 |
| c.326G > A | p.Gly109Glu | c.298del | p.His100MetfsTer12 |
| c.310_323del | p.Arg104GlyfsTer6 | c.298C > T | p.His100Tyr |
| c.317T > A | p.Phe106Tyr | c.296_297del | p.Arg99ThrfsTer2 |
| c.307A > T | p.Lys103Ter | c.290_295delinsCCCG | p.Tyr97SerfsTer4 |
| c.301_303del | p.Glu101del | c.293G > A | p.Arg98Gln |
| c.302A > G | p.Glu101Gly | c.292C > T | p.Arg98Trp |
| c.314A > G | p.Lys105Arg | c.290dup | p.Tyr97Ter |
| c.280_284dup | p.Ala96ThrfsTer18 | c.262G > T | p.Ala88Ser |
| c.283G > A | p.Val95Met | c.262G > C | p.Ala88Pro |
| c.279G > A | p.Met93Ile | c.258_260del | p.Pro87del |
| c.278T > C | p.Met93Thr | c.257C > T | p.Thr86Met |
| c.270_271insT | p.Val91CysfsTer11 | c.257C > G | p.Thr86Arg |
| c.269dup | p.Val91SerfsTer11 | c.253T > C | p.Ser85Pro |
| c.269del | p.Leu90GlnfsTer22 | c.251T > C | p.Val84Ala |
| c.269T > G | p.Leu90Arg | c.250G > T | p.Val84Leu |
| c.269T > C | p.Leu90Pro | c.250G > C | p.Val84Leu |
| c.268C > G | p.Leu90Val | c.250G > A | p.Val84Met |
| c.263C > T | p.Ala88Val | c.247_249del | p.Phe83del |
| c.263C > G | p.Ala88Gly | c.247T > A | p.Phe83Ile |
| c.263C > A | p.Ala88Glu | c.246C > G | p.Ile82Met |
| c.241C > G | p.Leu81Val | c.232dup | p.Ala78GlyfsTer24 |
| c.239A > T | p.Gln80Leu | c.232G > T | p.Ala78Ser |
| c.239A > G | p.Gln80Arg | c.232G > A | p.Ala78Thr |
| c.239A > C | p.Gln80Pro | c.231G > A | p.Trp77Ter |
| c.236_239delinsAGATCCG | p.Leu79_Gln80delinsGlnIleArg | c.230G > A | p.Trp77Ter |
| c.238C > T | p.Gln80Ter | c.229T > C | p.Trp77Arg |
| c.238C > A | p.Gln80Lys | c.227T > C | p.Leu76Pro |
| c.236T > C | p.Leu79Pro | c.224G > A | p.Arg75Gln |
| c.235del | p.Leu79CysfsTer3 | c.223C > T | p.Arg75Trp |
| c.235C > G | p.Leu79Val | c.2 18A > G | p.His73Arg, |
| c.217C > T | p.His73Tyr | c.195C > A | p.Tyr65Ter |
| c.212T > C | p.Ile71Thr | c.194A > G | p.Tyr65Cys |
| c.212T > A | p.Ile71Asn | c.193T > C | p.Tyr65His |
| c.209C > T | p.Pro70Leu | c.192C > A | p.Cys64Ter |
| c.208C > T | p.Pro70Ser | c.176_191del | p.Gly59AlafsTer18 |
| c.208C > G | p.Pro70Ala | c.191G > A | p.Cys64Tyr |
| c.200A > G | p.His67Arg | c.188T > C | p.Val63Ala |
| c.196G > C | p.Asp66His | c.187del | p.Val63CysfsTer19 |
| c.196G > A | p.Asp66Asn | c.187G > T | p.Val63Leu |
| c.195C > G | p.Tyr65Ter | c.187G > A | p.Val63Met |
| c.184_185insT | p.Asn62IlefsTer40 | c.169C > T | p.Gln57Ter |
| c.181A > C | p.Lys61Gln | c.167del | p.Leu56ArgfsTer26 |
| c.176del | p.Gly59AlafsTer23 | c.167T > C | p.Leu56Pro |
| c.176G > T | p.Gly59Val | c.164C > A | p.Thr55Asn |
| c.176G > C | p.Gly59Ala | c.163A > C | p.Thr55Pro |
| c.176G > A | p.Gly59Asp | c.162C > A | p.Asn54Lys |
| c.175G > C | p.Gly59Arg | c.161A > T | p.Asn54Ile |
| c.175G > A | p.Gly59Ser | c.161A > G | p.Asn54Ser |
| c.173C > G | p.Pro58Arg | c.160A > C | p.Asn54His |
| c.172C > G | p.Pro58Ala | c.155_158del | p.Val52AlafsTer29 |
| c.158G > A | p.Cys53Tyr | c.139G > T | p.Glu47Ter |
| c.157T > C | p.Cys53Arg | c.139G > C | p.Glu47Gln |
| c.154G > C | p.Val52Leu | c.139G > A | p.Glu47Lys |
| c.153del | p.Phe51LeufsTer31 | c.138T > G | p.Asp46Glu |
| c.149A > C | p.Asp50Ala | c.136G > A | p.Asp46Asn |
| c.148G > T; | p.Asp50Tyr | c.134G > A | p.Gly45Glu |
| c.148G > A | p.Asp50Asn | c.132G > C | p.Trp44Cys |
| c.147del | p.Asp50ThrfsTer32 | c.132G > A | p.Trp44Ter |
| c.146C > T | p.Ala49Val | c.131G > T | p.Trp44Leu |
| c.138_143del | p.Asp46_Gln48delinsGlu | c.131G > C | p.Trp44Ser |
| c.131G > A | p.Trp44Ter | c.109G > C | p.Val37Leu |
| c.125_127del | p.Glu42del | c.109G > A | p.Val37Ile |
| c.127G > A | p.Val43Met | c.107T > C | p.Leu36Pro |
| c.124G > A | p.Glu42Lys | c.104T > G | p.Ile35Ser |
| c.119C > T | p.Ala40Val | c.102G > A | p.Met34Ile |
| c.119C > G | p.Ala40Gly | c.101T > C | p.Met34Thr |
| c.119C > A | p.Ala40Glu | c.100A > T | p.Met34Leu |
| c.118G > T | p.Ala40Ser | c.100A > G | p.Met34Val |
| c.110T > C | p.Val37Ala | c.99del | p.Met34Ter |
| c.109G > T | p.Val37Phe | c.101 T > G | p.Met34Arg |
| c.98T > C | p.Ile33Thr | c.85_87del | p.Phe29del |
| c.98T > A | p.Ile33Asn | c.82C > A | p.Leu28Ile |
| c.95G > T | p.Arg32Leu | c.71G > A | p.Trp24Ter |
| c.95G > A | p.Arg32His | c.31_68del | p.Gly11LeufsTer24 |
| c.94C > T | p.Arg32Cys | c.62G > A | p.Gly21Glu |
| c.94C > A | p.Arg32Ser | c.51_62delinsA | p.Thr18LysfsTer26 |
| c.93del | p.Arg32AlafsTer3 | c.61G > A | p.Gly21Arg |
| c.91T > A | p.Phe31Ile | c.60T > G | p.Ile20Met |
| c.89T > A | p.Ile30Asn | c.59T > C | p.Ile20Thr |
| c.88del | p.Ile30PhefsTer5 | c.56G > C | p.Ser19Thr |
| c.88A > G | p.Ile30Val | c.53C > T | p.Thr18Ile |
| c.50C > T | p.Ser17Phe | c.35dup | p.Val13CysfsTer35 |
| c.50C > A | p.Ser17Tyr | c.35del | p.Gly12ValfsTer2 |
| c.47A > G | p.His16Arg | c.35G > T | p.Gly12Val |
| c.31_44del | p.Gly11ThrfsTer32 | c.35G > A | p.Gly12Asp |
| c.44A > C | p.Lys15Thr | c.34G > T | p.Gly12Cys |
| c.42C > G | p.Asn14Lys | c.34G > C | p.Gly12Arg |
| c.40A > T | p.Asn14Tyr | c.32G > A | p.Gly11Glu |
| c.40A > G | p.Asn14Asp | c.29T > C | p.Leu10Pro |
| c.37G > A | p.Val13Met | c.28del | p.Leu10TrpfsTer4 |
| c.24G > A | p.Thr8= | c.28_29delinsTG | p.Leu10Trp |
| c.23C > T | p.Thr8Met | c.7T > C | p.Trp3Arg |
| c.20A > C | p.Gln7Pro | c.1A > G | p.Met1? |
| c.19C > T | p.Gln7Ter | c.−1G > A | N/A |
| c.17T > C | p.Leu6Pro | c.−22−2A > C | N/A |
| c.11del | p.Gly4AlafsTer10 | c.−22−6T > C | N/A |
| c.9G > A | p.Trp3Ter | c.−23+1G > A | N/A |
| c.7dup | p.Trp3LeufsTer45 | c.−23G > T | N/A |
| c.37dup | p.Val13GlyfsTer35 | c.475G > T | p.Asp159Tyr |

Aspects of the present disclosure relate to methods of treating hearing loss (e.g., DFNB1) by delivering a functional gene product (e.g., GJB2 protein) using gene therapy (e.g., rAAV encoding GJB2 protein) to a target cell (e.g., cells that normally express GJB2, such as fibrocytes and supporting cells of the organ or Corti and nearby regions), which comprise one or more mutations in at least one alleles in a relevant gene (e.g., GJB2) that results in the absence or malfunction of the gene product.

Aspects of the invention relate to certain protein-encoding transgenes (e.g., GJB2) that when delivered to a subject are effective for treating hearing loss (e.g., DFNB1). In some embodiments, the subject has or is suspected of having hearing loss. In some embodiments, the hearing loss is associated with a mutation in the GJB2 gene. In some embodiments, the hearing loss is associated with a mutation in the GJB2 gene listed in Table 2 (above). In some embodiments, the subject is diagnosed with DFNB1.

Accordingly, methods and compositions described by the disclosure are useful, in some embodiments, for the treatment of DFNB1associated with one or more mutations or deletions in the GJB2 gene.

Methods for delivering a transgene (e.g., GJB2) to a subject are provided by the disclosure. The methods typically involve administering to a subject an effective amount of an isolated nucleic acid encoding a GJB2 protein, or a rAAV comprising a nucleic acid for expressing GJB2.

In some embodiments, the GJB2 mutations are, but are not limited to, point mutations, missense mutations, nonsense mutations, insertions, and deletions. In some embodiments, the GJB2 gene mutations associated with DFNB1 include, but are not limited to, mutations in Table 2. In some embodiments, the mutation in GJB2 gene is c.101T>C. In some embodiments, the mutation in GJB2 gene is 35DelG. The GJB2 mutation in a subject (e.g., a subject having or suspected of having DFNB1 associated with a deletion or mutation of GJB2 gene) may be identified from a sample obtained from the subject (e.g., a DNA sample, RNA sample, blood sample, or other biological sample) by any method known in the art. For example, in some embodiments, a nucleic acid (e.g., DNA, RNA, or a combination thereof) is extracted from a biological sample obtained from a subject and nucleic acid sequencing is performed in order to identify a mutation in the GJB2 gene. In some embodiments, a mutation in the GJB2 gene is detected indirectly, for example, by quantifying GJB2 protein expression (e.g., by Western blot) or function (e.g., by analyzing structure, function, etc.), or by direct sequencing of the DNA and comparing the sequence obtained to a control DNA sequence (e.g., a wild-type GJB2 DNA sequence).

In some aspects, the disclosure provides a method for treating DFNB1 in a subject in need thereof, the method comprising administering to a subject having or suspected of having DFNB1 a therapeutically effective amount of an isolated nucleic acid, or a rAAV encoding a transgene (e.g., GJB2). In some embodiments, the rAAV encoding a transgene (e.g., GJB2) is injected through injections to the round window membrane of the inner ear, as described by the disclosure. In some aspects, the present disclosure provides an isolated nucleic acid or an rAAV encoding a transgene (e.g., GJB2), or pharmaceutical compositions thereof, for use in the manufacturing of a medicament in a therapy. In some aspects, the present disclosure provides an isolated nucleic acid or an rAAV encoding a transgene (e.g., GJB2), or pharmaceutical compositions thereof, for use in the manufacturing of a medicament for treating hearing loss and/or deafness associated with the GJB2 gene. In some aspects, the present disclosure provides an isolated nucleic acid or an rAAV encoding a transgene (e.g., GJB2), or pharmaceutical compositions thereof, for use in the manufacturing of a medicament for treating non-syndromic deafness and/or hearing loss (DFNB1).

An “effective amount” of a substance is an amount sufficient to produce a desired effect. In some embodiments, an effective amount of an isolated nucleic acid (e.g., an isolated nucleic acid comprising a transgene encoding GJB2 protein) is an amount sufficient to transfect (or infect in the context of rAAV mediated delivery) a sufficient number of target cells of a target tissue of a subject. In some embodiments, the target tissue is cochlear (e.g., connective tissue cells of the cochlea and supporting cells of the organ of Corti and nearby regions as described herein). In some embodiments, an effective amount of an isolated nucleic acid (e.g., which may be delivered via an rAAV) may be an amount sufficient to have a therapeutic benefit in a subject, e.g., to increase or supplement the expression of a gene or protein of interest (e.g., GJB2 protein), to improve in the subject one or more symptoms of the disease (e.g., a symptom or sign of DFNB1), etc. The effective amount will depend on a variety of factors, such as, for example, the species, age, weight, health of the subject, and the tissue to be targeted, and may thus vary among subjects and tissue as described elsewhere in the disclosure. In some embodiments, an effective amount of a rAAV may be an amount sufficient to produce a stable somatic transgenic animal model.

An effective amount may also depend on the rAAV used. The invention is based in part on the recognition that a rAAV comprising capsid proteins having a particular serotype (e.g., AAV9.PHP.B or AAV-S) mediates more efficient transduction of cochlear (e.g., inner hair cells, out hair cells) tissue than a rAAV comprising capsid proteins having a different serotype.

In certain embodiments, the effective amount of rAAV is 10¹⁰, 10¹¹, 10¹², 10¹³, or 10¹⁴ genome copies per kg. In certain embodiments, the effective amount of rAAV is 10¹⁰, 10¹¹, 10¹², 10¹³, 10¹⁴, or 10¹⁵ genome copies per subject.

An effective amount may also depend on the mode of administration. For example, targeting a cochlear (e.g., connective tissue cells of the cochlea and supporting cells of the organ of Corti and nearby regions) tissue by injection through the round window membrane of the inner ear may require different (e.g., higher or lower) doses, in some cases, than targeting a cochlear (e.g., connective tissue cells of the cochlea and supporting cells of the organ of Corti and nearby regions) tissue by another method (e.g., systemic administration, topical administration). Thus, in some embodiments, the injection is injection through round window membrane of the inner ear. In some embodiments, administration is topical administration (e.g., topical administration to an ear). In some embodiments, the injection is posterior semicircular canal injection. In some cases, multiple doses of a rAAV are administered.

Without wishing to be bound by any particular theory, efficient transduction of cochlear cells (e.g., connective tissue cells of the cochlea and supporting cells of the organ of Corti and nearby regions as described herein) by rAAV described herein may be useful for the treatment of a subject having a hereditary hearing loss (e.g., DFNB1). In some embodiments, the composition and method described herein may be useful to treat other GJB2-associated diseases. GJB2-associated diseases, as used herein, refer to conditions and/or disorders caused by GJB2 mutations (e.g., loss of function mutations). Non-limiting GJB2-associated disease include Deafness, autosomal recessive 1A, Deafness, autosomal dominant 3A, DFNB1, Keratitis-ichthyosis-deafness (KID), Ichthyosis, hystrix-like-deafness (HID), Palmoplantar keratoderma-deafness (PPK), Porokeratotic eccrine ostial and dermal duct nevus, Vohwinkel, Burt-Pumphrey, Unususal mucocutaneous-deafness (see, e.g., Srinivas et al., Human diseases associated with connexin mutations, Biochimica et Biophysica Acta (BBA)—Biomembranes, Volume 1860, Issue 1, January 2018, Pages 192-201; Lossa et al., GJB2 Gene Mutations in Syndromic Skin Diseases with Sensorineural Hearing Loss, Curr Genomics. 2011 November; 12(7): 475-785)

Accordingly, methods and compositions for treating hereditary hearing loss are also provided herein. In some aspects, the disclosure provides a method for treating hereditary hearing loss (e.g., DFNB1) or any other GJB2-associated diseases described herein, the method comprising administering to a subject having or suspected of having hereditary hearing loss an effective amount of rAAV, wherein the rAAV comprises (i) a capsid protein having a serotype of AAV9.PHP.B, or AAV-S, and (ii) an isolated nucleic acid comprising two adeno-associated virus (AAV) inverted terminal repeats (ITRs) flanking an expression cassette, wherein the expression cassette comprises a promoter operably linked to a nucleotide sequence encoding a GJB2 gene regulatory element (GRE), and a nucleotide sequence encoding a gap junction beta 2 (GJB2) protein

In some embodiments, the rAAV (e.g., rAAV encoding GJB2) can be administered to a patient (e.g., a patient with DFNB1) at the age of 1 day, 10 days, 1 month, 3 months, 6 months, 1 year, 2 years, 3 years, 5 years, 6 years, 7 years, 8 years, 9, years, 10 years, 11 years, 12 years, 13 years, 14 years, 15 years, 16 years, 17 years, 18 years, or older. In some embodiments, the patient is an infant, a child, or an adult. In some embodiments, the window of treating GJB2-associated diseases (e.g., DFNB1) is normally from birth to pre-school age (e.g., from birth to 1 year old, from 1 to 2 years old, from 2-3 years old, from 3-4 years old, from 4-5 years old, or from 5-6 years old). In some embodiments, the rAAV (e.g., rAAV encoding GJB2) is administered to the patient (e.g., patients with DFNB1) once in a life-time, every 10 years, every 5 years, every 2 years, every year, every 6 months, every 3 months, every month, every two weeks, or every week. In other embodiments, the administration of the rAAV (e.g., rAAV encoding GJB2) is administered to the patient (e.g., patients with DFNB1) in combination with other known treatment methods for GJB2-associated diseases (e.g., DFNB1).

### V. Kits and Related Composition

The agents described herein may, in some embodiments, be assembled into pharmaceutical or research kits to facilitate their use in therapeutic, or research applications. A kit may include one or more containers housing the components (e.g., nucleic acids, rAAV) of the disclosure and instructions for use. Specifically, such kits may include one or more agents described herein, along with instructions describing the intended application and the proper use of these agents. In certain embodiments, agents in a kit may be in a pharmaceutical formulation and dosage suitable for a particular application and for a method of administration of the agents. Kits for research purposes may contain the components in appropriate concentrations or quantities for performing various experiments.

In some embodiments, the instant disclosure relates to a kit for administering a rAAV as described herein. In some embodiments, the kit comprises a container housing the rAAV, and devices (e.g., syringe) for extracting the rAAV from the housing. In some embodiments, the device for extracting the rAAV from the housing is also used for administration (e.g., injection).

In some embodiments, the instant disclosure relates to a kit for producing a rAAV, the kit comprising a container housing an isolated nucleic acid comprising a transgene encoding a protein (e.g., GJB2). In some embodiments, the kit further comprises a container housing an isolated nucleic acid encoding an AAV capsid protein, for example, an AAV.PHP.B capsid protein or an AAV-S capsid protein. In some embodiments, the kit further comprises vectors encoding the rep/cap genes, and the host for producing the rAAV.

In some embodiments, the instant disclosure relates to a kit for treating hearing loss (e.g., DFNB1). In some embodiments, the kit is for delivering a functional (e.g., DFNB1) to a target cell (e.g., connective tissue cells of the cochlea and supporting cells of the organ of Corti and nearby regions as described herein) using gene therapy (e.g., rAAV described herein).

The kit may be designed to facilitate use of the methods described herein by researchers and can take many different forms. Each of the compositions of the kit, where applicable, may be provided in liquid form (e.g., in solution), or in solid form (e.g., a dry powder). In certain cases, some of the compositions may be constitutable or otherwise processable (e.g., to an active form), for example, by the addition of a suitable solvent or other medium (for example, water or a cell culture medium), which may or may not be provided in the kit. As used herein, “instructions” can include a component of instruction and/or promotion, and typically involve written instructions on or associated with the packaging. Instructions also can include any oral or electronic instructions provided in any manner such that a user will clearly recognize that the instructions are to be associated with the kit, for example, audiovisual (e.g., videotape, DVD, CD-ROM, website links for downloadable file, etc.), Internet, and/or web-based communications, etc. The written instructions may be in a form prescribed by a governmental agency regulating the manufacture, use, or sale of pharmaceuticals or biological products, which instructions can also reflect approval by the agency of manufacture, use, or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing the rAAV described herein. The rAAV may be in the form of a liquid, gel, or solid (powder). The rAAV may be prepared sterilely, packaged in a syringe, and shipped refrigerated. Alternatively, the rAAV may be housed in a vial or other container for storage. A second container may have other agents prepared sterilely. Alternatively, the kit may include the rAAV premixed and shipped in a syringe, vial, tube, or other container.

### VI. General Techniques

The practice of the present invention will employ, unless otherwise indicated, conventional techniques of molecular biology (including recombinant techniques), microbiology, cell biology, biochemistry, and immunology, which are within the skill of the art. Molecular Cloning: A Laboratory Manual, second edition (Sambrook, et al., 1989) Cold Spring Harbor Press; Oligonu□leotide Synthesis (M. J. Gait, ed., 1984); Methods in Mole□ular Biology, Humana Press; Cell Biology: A Laboratory Notebook (J. E. Cellis, ed., 1998) A□ademi□Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Pro□edures (A. Doyle, J. B. Griffiths, and D. G. Newell, eds., 1993-8) J. Wiley and Sons; Methods in Enzymology (A□ademi□Press, In□); Handbook of Experimental Immunology (D. M. Weir and C. C. Bla□kwell, eds.); Gene Transfer Ve□ors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Proto□ols in Immunology (J. E. Coligan et al., eds., 1991); Short Proto□ols in Mole□ular Biology (Wiley and Sons, 1999); Immunobiology (C. A. Janeway and P. Travers, 1997); Antibodies (P. Fin□h, 1997); Antibodies: a pra□ti□al approa□h (D. Catty., ed., IRL Press, 1988-1989); Mono□lonal antibodies: a pra□ti□al approa□h (P. Shepherd and C. Dean, eds., Oxford University Press, 2000); Using antibodies: a laboratory manual (E. Harlow and D. Lane (Cold Spring Harbor Laboratory Press, 1999)); The Antibodies (M. Zanetti and J. D. Capra, eds., Harwood Academic Publishers, 1995).

Without further elaboration, it is believed that one skilled in the art can, based on the present disclosure, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

Exemplary embodiments of the invention will be described in more detail by the following examples. These embodiments are exemplary of the invention, which one skilled in the art will recognize is not limited to the exemplary embodiments.

### EXAMPLES

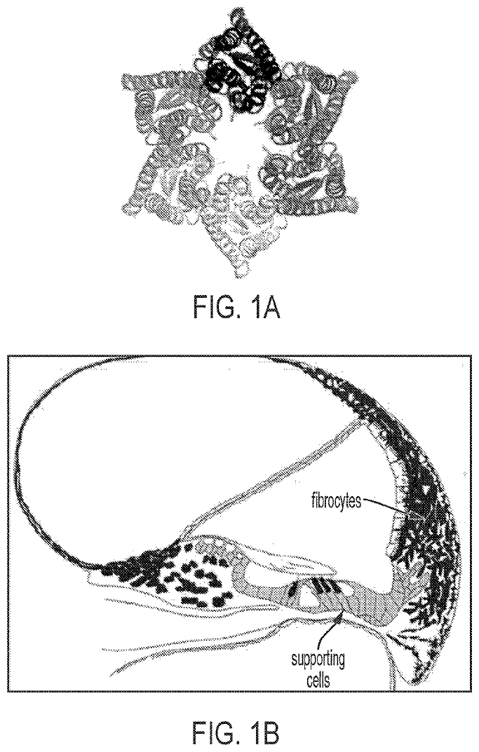

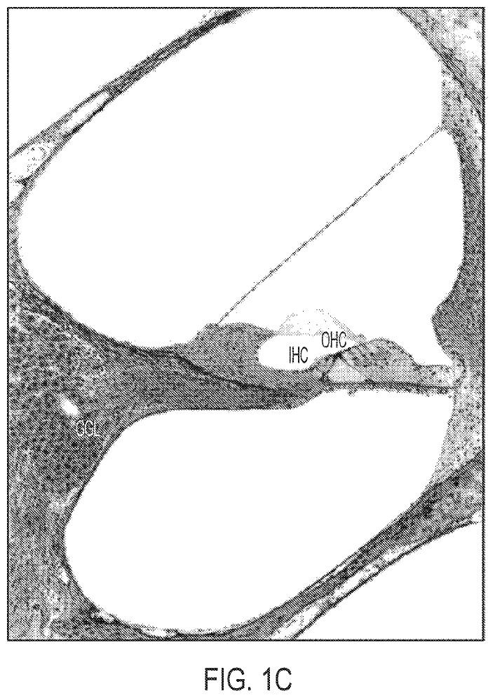

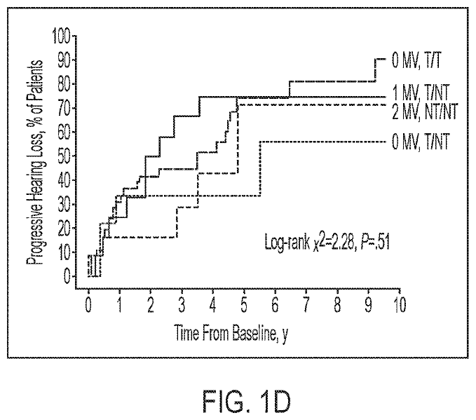

Hearing impairment of genetic origin occurs in about 1 in 1,000 births; most are autosomal recessive and nonsyndromic. Although over 70 different deafness genes have been identified, nearly half of all cases of severe to profound autosomal recessive nonsyndromic hearing loss result from mutations in just one gene: GJB2, encoding the gap-junction protein connexin26, which contains six subunits to form a hemichannel. Each subunit has four transmembrane helices, which assemble in the plane of the membrane to form a large central pore (FIG. 1A). GJB2 hemichannels from adjacent cells join to create a channel from the cytoplasm of one cell to the cytoplasm of the other. Gap junctions are formed by hundreds or thousands of channels packed in a junctional plaque.

In the cochlea, GJB2 is expressed in two cell groups: an epithelial system comprising supporting cells of the organ of Corti, epithelial cells of the inner and outer sulcus, and interdental cells; and a cytoplasmic system comprising fibrocytes of the lateral wall and suprastrial zone, basal cells of the stria vascularis, and supralimbal dark cells (See, e.g., Kikuchi et al., (1995) Gap junctions in the rat cochlea: immunohistochemical and ultrastructural analysis. Anat Embryol (Berl) 191:101-118). It is not expressed in hair cells. In the cochlea, the epithelial system is largely post-mitotic. In contrast, fibroblasts of the cytoplasmic system turn over slowly, but there is some cell division observed with BrdU labeling (Lang et al., 2002; Li et al., 2017). Structure of the cochlea and the fibrocytes/Corti supporting cell network are shown in FIGS. 1A-1B.

GJB2 expression is critical for cochlear function. For example, the K⁺ that enters hair cells through transduction channels and leaves through basal K⁺ channels is shuttled away from the organ of Corti by the epithelial system and conveyed by the cytoplasmic system to the stria, where it is pumped back into the endolymph. Further, GJB2 plays a role in development of the cochlea, as mice lacking GJB2 in the inner ear have reduced endocochlear potential and profound apoptotic loss of hair cells and supporting cells by P30, even though hair cells do not express Gjb2 (Cohen-Salmon et al., 2002; Wang et al., 2009; Sun et al., 2009; Crispino et al., 2011; Johnson et al., 2017). If Gjb2 is deleted after P6, the phenotype is much milder (Chang et al., 2015). However there remains a long-term requirement for GJB2: hair cell loss occurs after months even with deletion as late as P14 (Ma et al., 2020). Not wishing to be bound by the theories described herein, GJB2's function in shuttling K⁺ may be related in its role in development of the cochlea: If K⁺ is not carried away from hair cells by a gap junction network, K⁺ accumulation could depolarize hair cells, leading to Ca²⁺ influx and eventual cell death. The gap junction network may also be required to transport glucose and nutrients from blood vessels to the sensory epithelium and its absence could lead to cell death (Chang et al., 2008; Mammano, 2019).

Loss of GJB2 expression underlies a disorder termed Nonsyndromic Hearing Loss and Deafness, (DFNB1), characterized by recessive, mild-to-profound sensorineural hearing impairment (Kelsell et al., 1997; Kenna et al., 2010). Over 100 mutations have since been described in patients, but nearly 60% of patients have a single base deletion (35delG) leading to a frameshift and stop (Kenna et al., 2010). In the United States alone, about 3,500 children are born each year with two mutations in the causative gene, GJB2 (Kelsell et al., 1997; Zelante et al., 1997; Azaiez et al., 2018). Many are born with profound hearing loss, which is probably irreversible even at birth. Two-thirds have some residual hearing at birth and the majority of those lose hearing over the next few years, suggesting that a window exists for therapeutic intervention (Kenna et al., 2010). There are thus 5-10,000 preschool-age children who are potential candidates for treatment of DFNB1 (FIG. 1D).

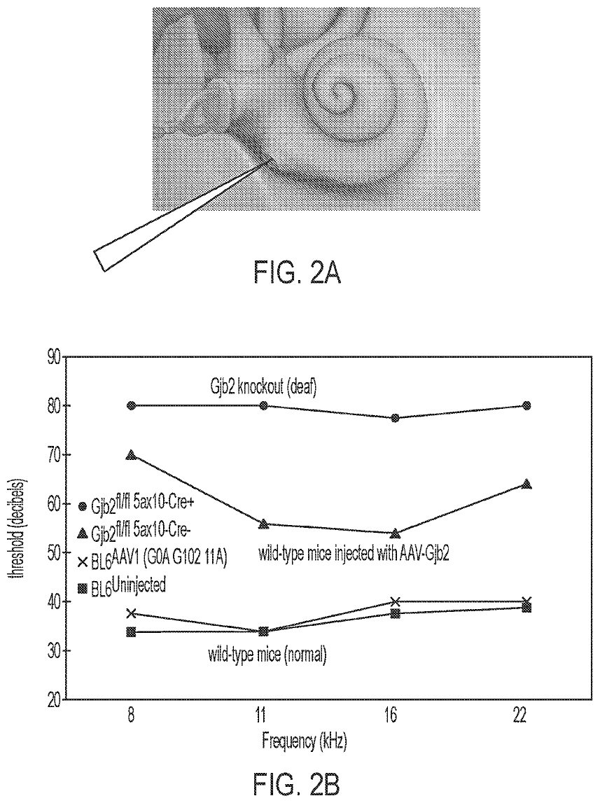

Because the cochlea is a surgically accessible and relatively immunoprotected environment, gene therapy using viral vectors is an attractive approach. The GJB2 coding sequence is small (˜680 bp) and will easily fit in an AAV vector. Although AAV does not insert into the genome and is diluted in dividing cells, most cochlear cells do not divide and AAV can drive expression for decades or more. The injection of rAAV carrying the coding sequence of GJB2 is normally injected through the round window membrane (RWM) (FIG. 2A). However, previous trials of gene therapy failed to rescue hearing even though gene addition of GJB2 rescued cell survival and the gap junction network.

Surprisingly, it was found that indiscriminate expression of GJB2 in the cochlea compromises the function of hair cells and neurons even as it rescues function in the fibrocytes and supporting cells. Further, promiscuous expression of GJB2 in the inner ear damaged hearing of the wild-type mice (FIG. 2B).

Gap junctions create a low-resistance path between adjacent cells. Hair cells and neurons of the cochlea, however, rely on high-resistance membranes to generate depolarization with small transduction or synaptic currents. If either is electrically coupled to adjacent cells, the depolarization would be shunted and the signal to the brain lost. The surprising phenomenon of hearing loss caused by promiscuous GJB2 expression could be explained by indiscriminate gap-junction coupling of hair cells, which do not normally express GJB2. Therefore, effective gene therapy treatment should lead to cell-specific expression of exogenous GJB2 in cells that normally express the gene (e.g., fibrocytes and supporting cells) in order to rescue hearing in subjects with GJB2 mutations.

To achieve cell specific GJB2 expression, cis-regulatory elements of the GJB2 gene were evaluated. Large genomic deletions upstream of GJB2, from 130 to >300 kb, have been found to cause congenital profound deafness. Overlap analysis of these deletions reveals a shared region of ˜95 kb (FIG. 3A), suspected to house the critical enhancer(s) for GJB2 expression in the inner ear.

To identify the cis-regulatory enhancer of GJB2 in human patients, a combination of patient genomic data, ATAC-Seq and in vitro assays was used. Patients with suspected GJB2-related hearing loss were screened with either targeted genomic enrichment coupled with massively parallel sequencing or genome sequencing to search for non-coding disease-causing variants within the ˜95.4 kb window (FIG. 3B). The genotype and phenotype of patients who were screened with the OtoSCOPE panel were reviewed. The initial round of selection included all patients that were heterozygous for a known or predicted pathogenic variant in the GJB2 coding sequence and had a negative genetic diagnosis for their hearing loss. Next, the cohort of patients were refined based on phenotype. Patients carrying a loss-of-function mutation in trans with a mutation in the cis-regulatory element should have congenital severe to profound deafness. Families with recessive deafness that have linkage/allele segregation to the GJB2 locus and absence of coding variants in GJB2 were also studied.

After sequencing, the data was analyzed by a custom bioinformatics pipeline following The Broad Institute's GATK best practices. Briefly, raw sequences were mapped to the genome using Burrows-Wheeler Aligner, followed by Picard to remove duplicates, Genome Analysis Tool Kit (GATK) for variant calling, and Ensembl Variant Effect Predictor and dbNSFP to annotate for variant annotation. After annotation, variants were filtered based on quality, minor allele frequency and location (within the ˜95 kb window). Variants were prioritized based on variants that fall within regulatory elements, as defined by the Encyclopedia of DNA Elements (ENCODE) and the Genotype-Tissue Expression. Over 100 patients were sequenced, and more than 200 candidate variants were identified. Roughly 5-10% of DFNB1 patients have a second disease-causing allele in a non-coding region.

In mice and non-human primates, ATAC-Seq (Assay for Transposase-Accessible Chromatin using Sequencing; Buenrostro et al., 2013) was used to identify enhancers for genes active in the cochlea. ATAC-Seq employs a hyperactive mutant Tn5 transposase that inserts sequencing adapters into open regions of the genome. The genomic DNA was then sequenced from the adapters to identify open chromatin.

Cochleae were dissected from neonatal mice at ages P2, P5 and P8, the time that the cochlea acquires normal function. One cochlea was dissected from an adult macaque monkey. This data set is an important contribution to studies of gene regulation in the cochlea. It can be used, for instance, to drive gene expression in specific cell types that are frequently impaired in both hereditary and acquired hearing loss, such as hair cells, the adjacent stem cells, and spiral ganglion neurons.

Eighteen candidate enhancers associated with the mouse Gjb2 gene were identified. FIG. 3C shows ˜200 kb of mouse genomic sequence in the region of the mouse Gjb2 gene; highlighted are regions with many ATAC-Seq reads. The subsequent studies focused on those enhancers that are near the mouse Gjb2 gene, which are conserved among mammalian species. FIG. 3C (top) shows the identification of mouse Gjb2 gene regulatory elements (GREs), in UCSC Genome Browser views of ATAC-Seq from mouse cochlea at developmental stages P2, P5 and P8, over ˜300 kb in the region of the mouse Gjb2 gene. Shaded regions mark regions containing putative GREs (Human and mouse reginal sequences containing GREs are listed in Table 1). X-axis is the genomic region on chr14 in the mouse genome. Y-axis is the number of reads from the ATAC-Seq that align to a specific region in the genome. Light blue highlight denotes regions of open chromatin, which are the hallmarks of transcriptionally active regions that are enriched for read pile up, suggesting higher activity in these regions. Regions A and B mark the transcriptionally active sequences within mouse Gjb2 itself. Regions C-M are regions that are transcriptionally active around Gjb2 that might be part of a cis-regulatory network. GJB2 GRE sequences were identified with the regional sequences listed in Table 1. FIG. 3C (bottom) shows transcriptionally active regions in and around the light-blue shaded regions that have been identified as specific mouse Gjb2 GREs (GREs 2, 3, 5, 7, and 9). Human GJB2 GRE sequences were identified in silico by modeling the mouse Gjb2 GREs. The nucleotide sequences of human GREs 1, 2, 3, 4, 5, 7 and 9 are set forth in Table 3, and were tested in subsequent experiments.

Further, the promoter, 5′ UTR and/or 3′ UTR of the GJB2 gene also contains native regulatory sequences. Constructs including the promoter, 5′ UTR and/or 3′ UTR were designed and tested for their capability in cell specific GJB2 expression. The constructs were packaged into rAAVs and injected into the inner ear of mice. The cell types expressing the marker gene were compared against cell types that express GJB2. For instance, a C15 vector was constructed to include 500 bp of the human GJB2 promoter, and 300 bp of the 5′ UTR, followed by a coding sequence for GFP and human GJB2 3′ UTR, (Vector C15 in FIG. 3D). The C15 vector packaged into rAAV using AAV9-PHP.B capsid, which is previously found to be effective in transducing many cochlear cell types (Gyorgy et al., 2018). The AAV9-PHP.B-C15 virus was injected into inner ears of P0 mouse pups. GJB2 expression was detected by immunofluorescent using an antibody targeting GJB2 (FIG. 3F, middle panel). Cells transduced with the AAV9-PHP.B-c15 vector and expressing the GFP marker gene under GJB2 enhancers are shown in the left panel. The expression pattern of GJB2 in the inner ear was consistent with what was reported by Kikuchi. In the right panel, IHCs and OHCs (indicated) are also identified by labeling actin with fluorescent phalloidin. In the right panel, IHCs and OHCs (indicated) are also identified by labeling actin with fluorescent phalloidin. Notably, AAV9-PHP.B-C15 is capable of efficiently transducing hair cells, but no GFP expression was observed in hair cells. This is likely because the Gjb2 enhancers are not active in hair cells. FIG. 3F shows a segment of the mouse cochlea, from the lateral wall (top) to the interdental cells (bottom). Cells transduced with the AAV9-PHP.B-C15 vector and expressing the GFP marker gene under Gjb2 enhancers are shown in the left panel. Cells normally expressing Gjb2 are shown in the middle panel. In the right panel, IHCs and OHCs (indicated) are also identified by labeling actin with fluorescent phalloidin. The expression pattern of GFP, which was driven by the c15 construct, is consistent with native Gjb2 expression reported in Kikuchi et al., 1995 using the same antibody against GJB2. Notably, c15 does not drive GFP expression in hair cells.

Further, other constructs (C20-C23) were designed to test exogenous GJB2 expression under a promiscuous chicken beta Actin (CBA) promoter. In C20 vector, the human GJB2 coding sequence was driven by the CBA promoter (FIG. 3E, vector C20). C20 vector was packaged into rAAVs and injected it into P0 cochleae in mice. GJB2 expression was confirmed in hair cells with immunofluorescence using the GJB2 antibody (FIG. 3G). Expression of GJB2 by hair cells would produce electrical coupling to adjacent supporting cells and short-circuit the normal sensory receptor potential. To test this theory, several other vectors were designed. C21 vector includes a CBA promoter operably linked to the human GJB2 coding sequence harboring a 35delG mutation. No active GJB2 protein can be produced by C21 vector. C22 vector includes a CBA promoter with no GJB2 coding sequence. C23 vector includes a CBA promoter driving the expression of human Clarin 1, which is a protein normally expressed by hair cells. The vectors were packaged into rAAVs using AAV1 or AAV9-PHP.B capsid. The rAAVs were injected into the inner ear of mice through the round window membrane at P1, and Auditory Brainstem Response (ABR) was measured at P30 (threshold at 8, 11 and 16 kHz averaged). As shown in FIG. 3H, uninfected wild-type mice had ABR thresholds near 30 dB, and saline mock injection did not change the ABR threshold in wild-type mice. GJB2 expression with a CBA promoter in either AAV1 or AAV9-PHP.B capsids elevated thresholds by 30-40 dB. For comparison, the conditional knockout Cre+, Gjb2^{fl/fl} mice had no response at the highest level tested (90 dB). Further, it was observed that mice injected with AAV9-PHP.B-C20 often showed neurological symptoms including seizures and often death. No lethality was observed in vector AAV9-PHP.B-C21 (expressed GJB2 with an inactivating mutation), AAV9-PHP.B-C22 (no GJB2 coding sequence), or AAV9-PHP.B-C23 (expressed Clarin 1, a normal hair-cell protein). Further, if the rAAV was diluted 10-or 100-fold prior to injection, no toxicity or lethality was observed with any of the vectors. It is possible that a small amount of rAAV encoding GJB2 was reaching the brain due to the brain tropism of AAV9-PHP.B, where electrical coupling of neurons is impairing neural regulation of homeostatic systems. This unexpectedly but dramatically illustrated the need to restrict GJB2 expression to the appropriate cells to reduce toxicity.

The Sox10-Cre+,Gjb2^{fl/fl} knockout mice have no response at the highest level tested (90 dB) (FIG. 3H). In the knockout, AAV1-CBA-GJB2 or AAV9-PHP.B-CBA-GJB2 rAAVs produced no rescue. A C70 construct was produced to test the enhancers in rescuing hearing. The C70 construct includes an AAV 5′ ITR, a GJB2 basal promoter, a GJB2 exon 1 5′ UTR, Kozak sequence, mouse or human GJB2 coding sequence, an optional HA tag, a GJB2 exon 2 3′ UTR, a WPRE, a bovine growth hormone poly A signal, and an AAV 3′ ITR. The C70 construct was packaged into rAAVs using AAV9-PHP.B capsid protein and injected into the inner ear of both wild-type mice and the Sox10-Cre+,Gjb2^{fl/fl} knockout mice. Gjb2 expression rescued hearing by 15-20 dB in Sox10-Cre+,Gjb2^{fl/fl} knockout mice. The same vector did not damage hearing in wild-type mice (FIG. 3H). FIGS. 3I-3L shows the map of the c70 vector plasmid encoding mouse GJB2 or human GJB2 with or without an HA tag. FIG. 3M shows schematics of vector c.70 encoding mouse GJB2 or human GJB2 with or without the HA tag. FIG. 3N shows additional vectors that were created and tested.

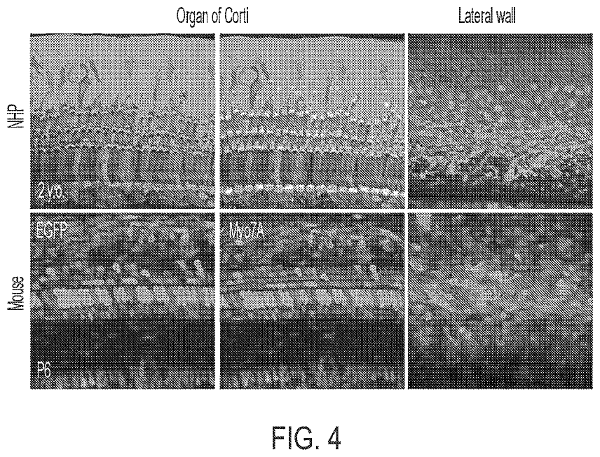

Moreover, other AAV capsid proteins having tropisms to inner ear cells were tested for their capability in delivering a transgene (e.g., GJB2 or GFP) to appropriate inner ear cells in both mouse and primates and rescuing hearing. AAV-S capsid protein, originally developed for brain tropism, showed good transduction of GJB2-expressing cells in both mouse and primate cochlea (FIG. 4). An rAAV comprising the AAV-S capsid protein and the c70 vector, which drives expression of GJB2 under the GJB2 basal promoter and 5′ UTR, was packaged. The AAV-S-C70 rAAV is injected into Gjb2 conditional knockout mice. The hearing of these mice is tested. The AAV-S-C70 rAAV is capable of rescuing hearing similarly to AAV9-PHP.B-C70 rAAV, or even better.

The AAV-S-C70 rAAV is injected into wild-type mice. The C70 vector includes an HA tag, which allows easy detection of GJB2 expression in the inner ear with an anti-HA antibody. It is expected that GJB2 expression is only detected in supporting cells of the organ of Corti and fibrocytes, which normally express GJB2. The hearing of the injected wild-type mice is also tested to assess GJB2-associated toxicity.

Further, the ability of AAV-S to transduce inner ear cells of non-human primates (NHP) was tested. An rAAV comprising an AAV-S capsid protein and a vector encoding GFP was injected into both ears of non-human primates. Animals were euthanized three weeks later and the cochleas prepared for histology. GFP expression is evaluated in the cochleas in these animals. Similar experiments in mice were carried out in parallel.

An AAV-S vector encoding GFP was injected into the inner ear of an adult mouse, using the posterior canal route (which robustly delivers vector throughout the inner ear in mouse). The animal was euthanized 20 days after the injection and the cochlea harvested.

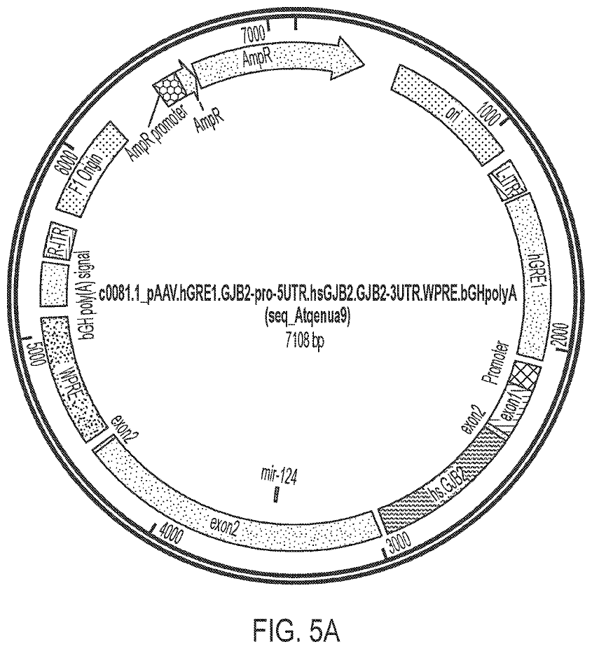

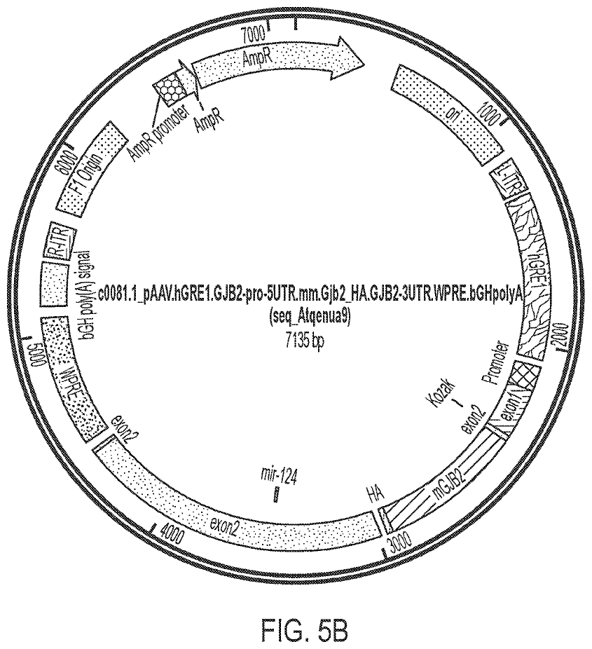

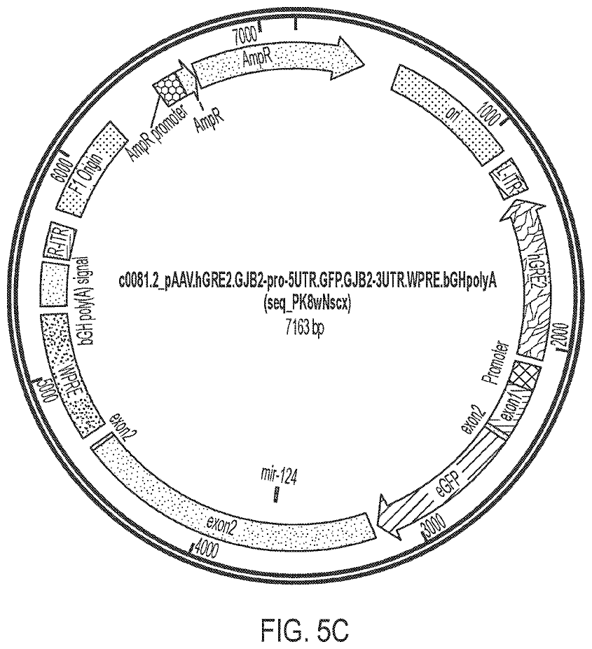

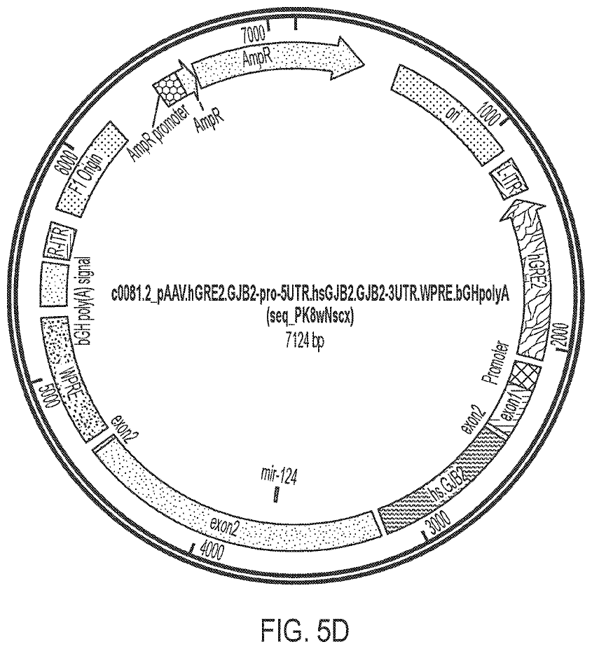

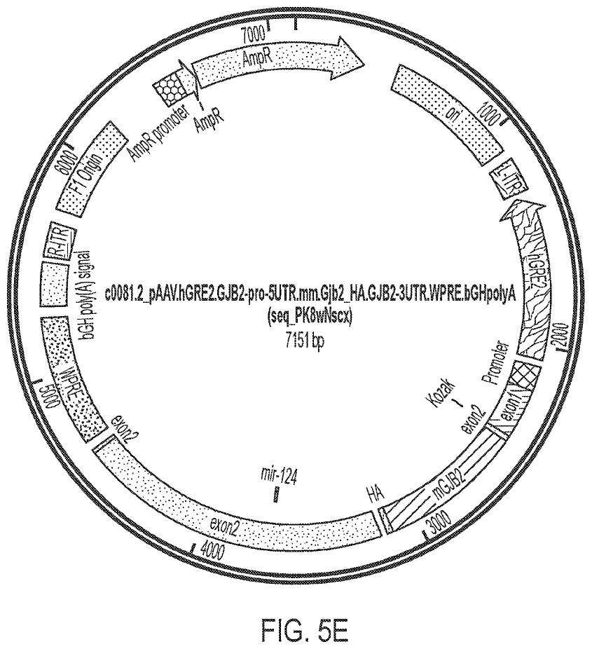

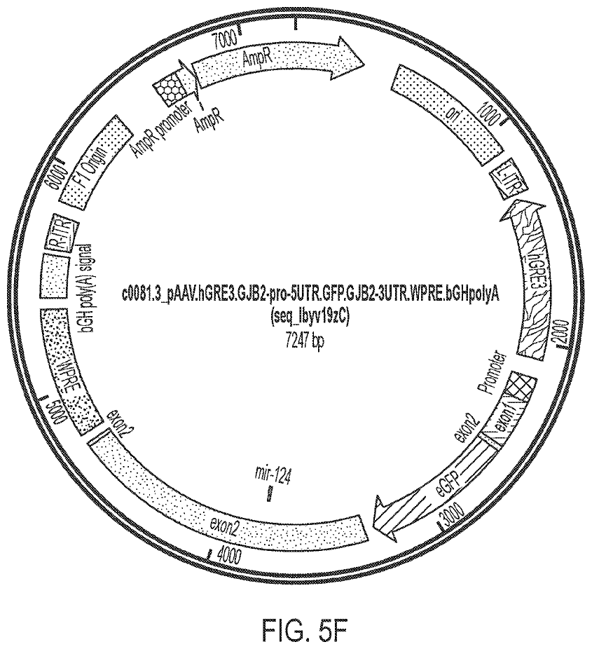

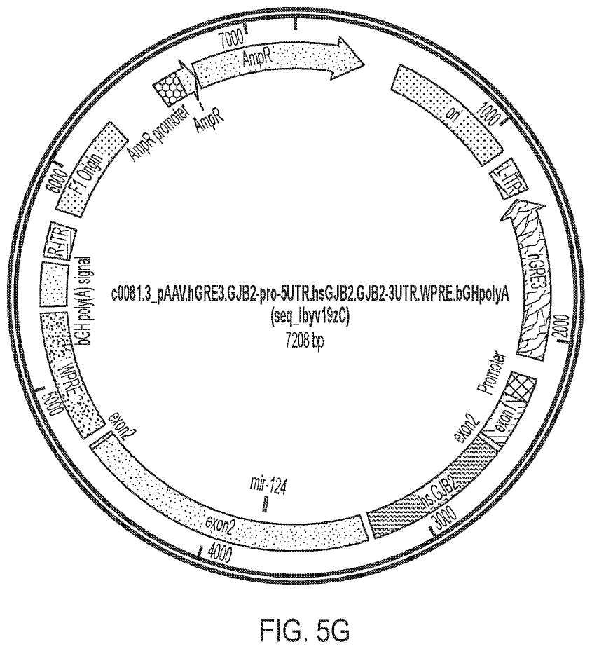

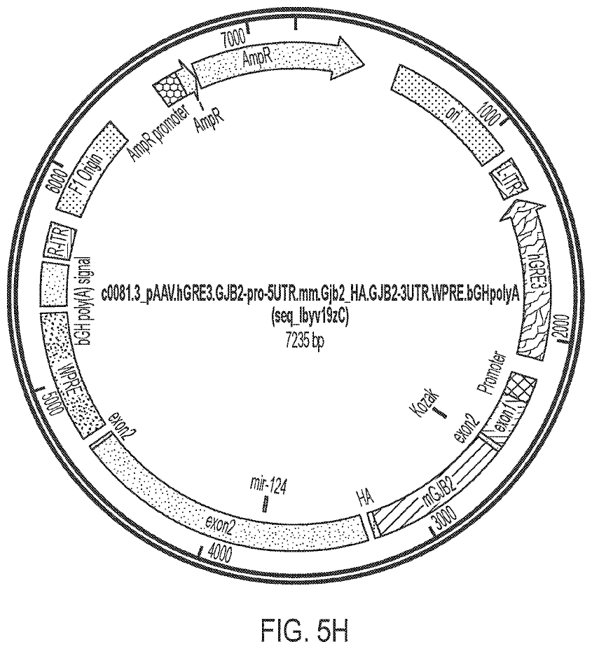

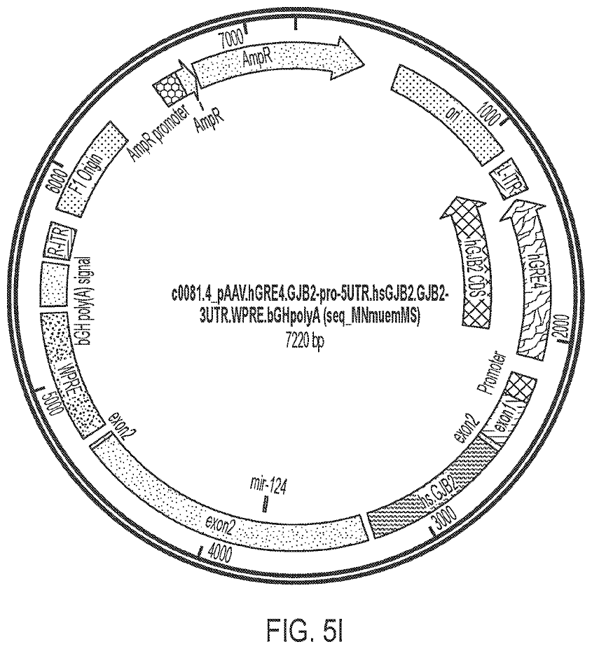

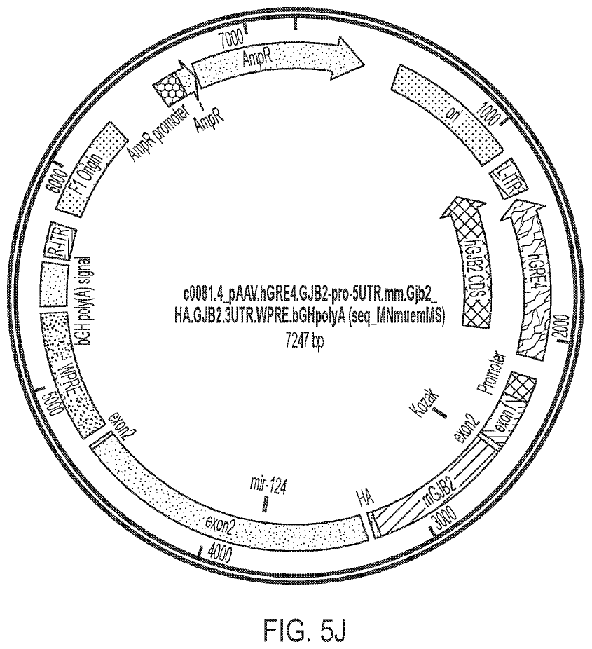

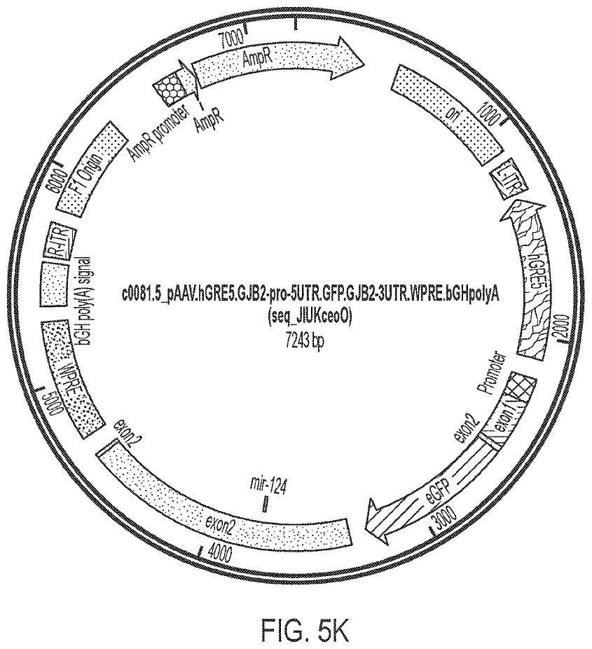

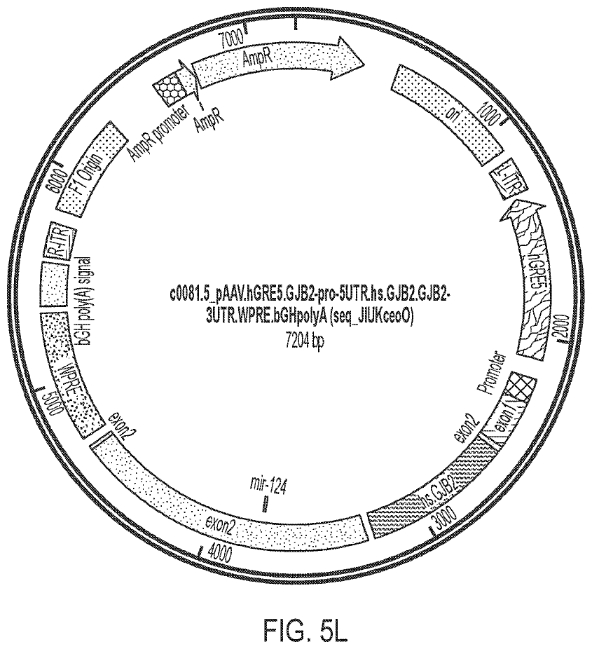

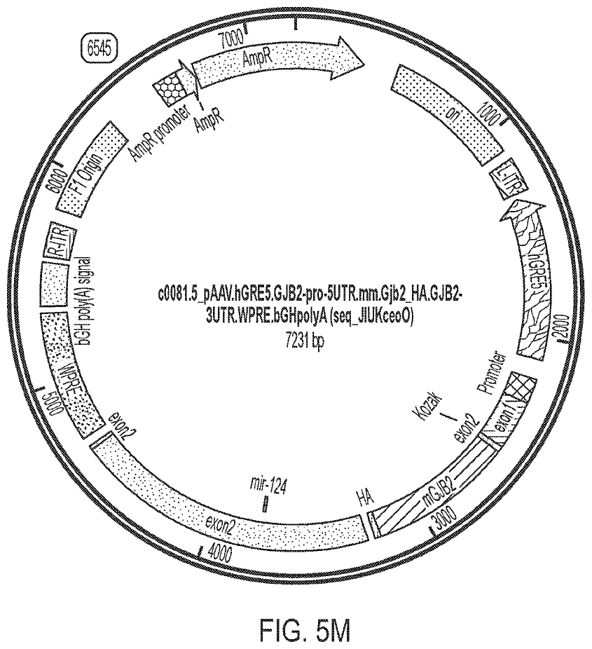

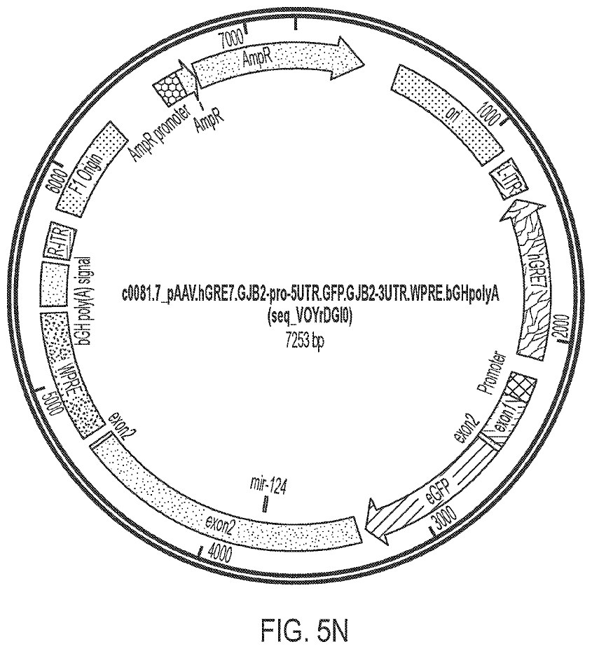

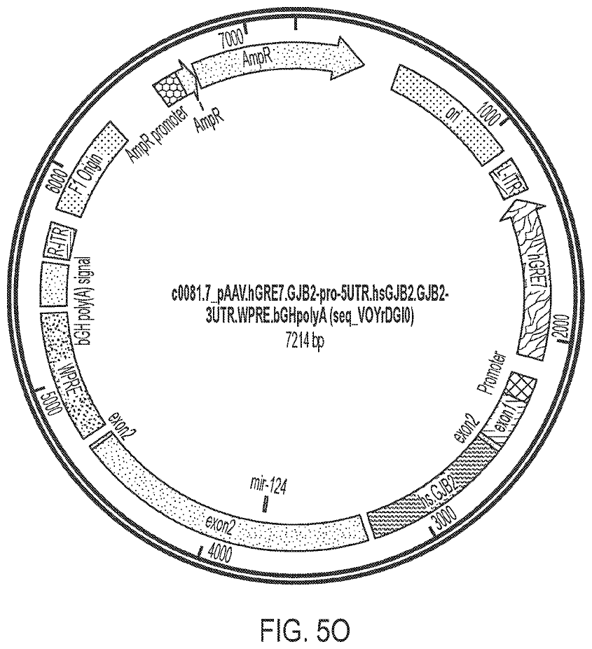

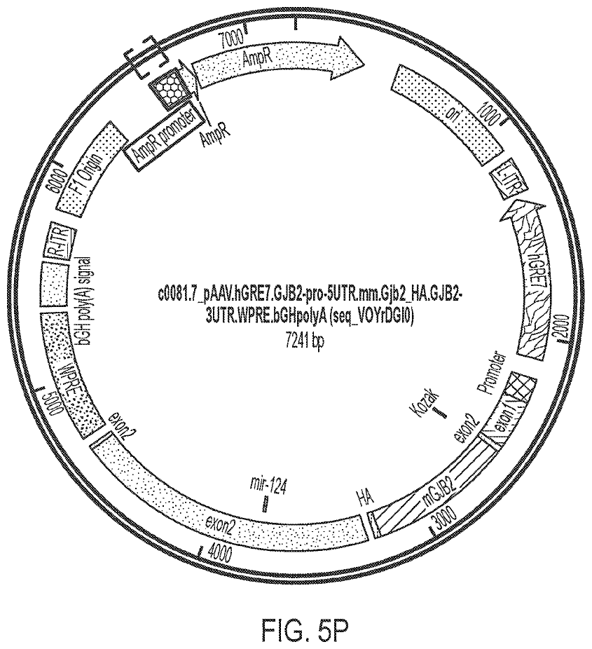

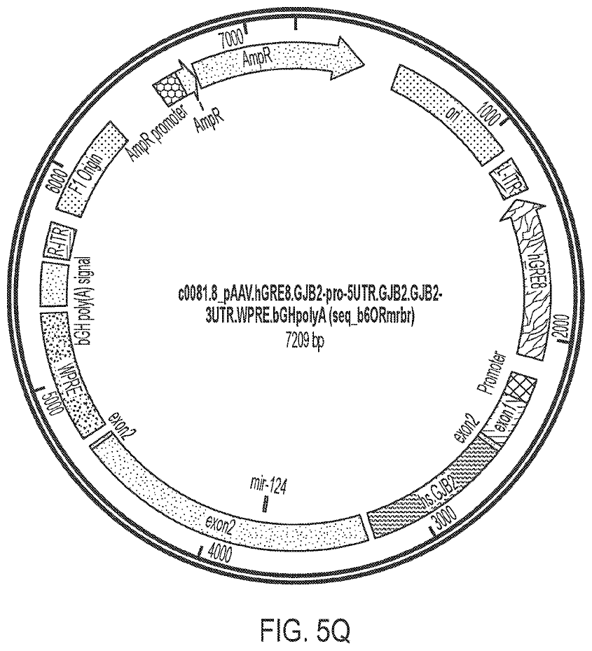

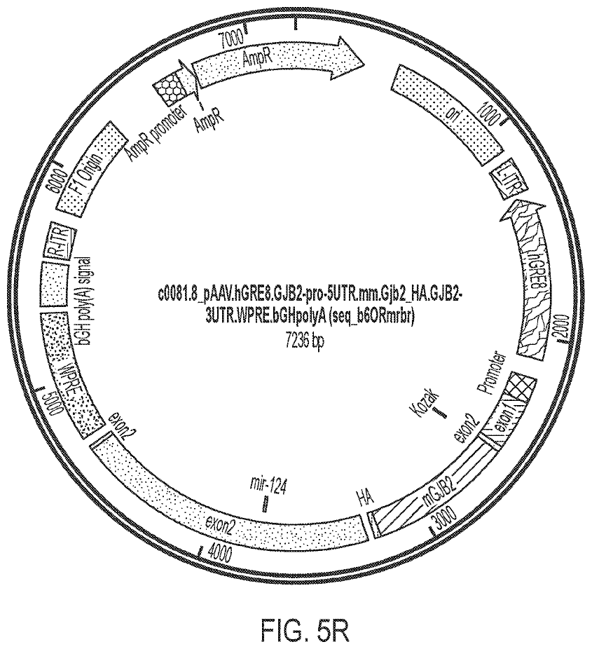

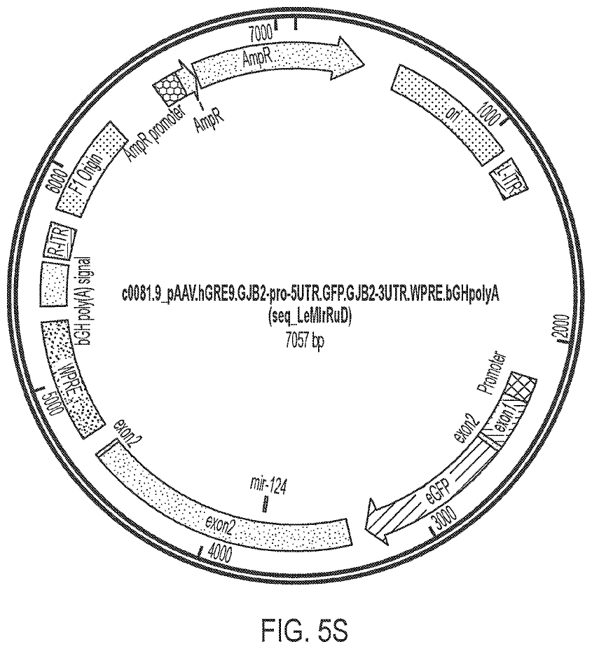

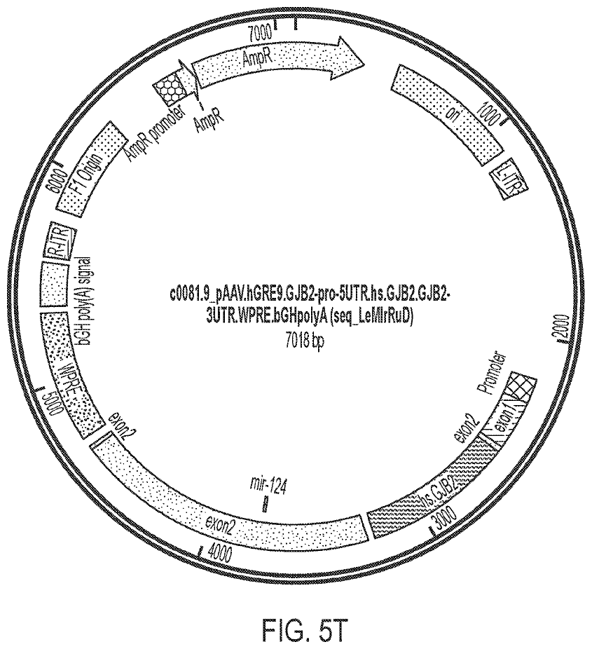

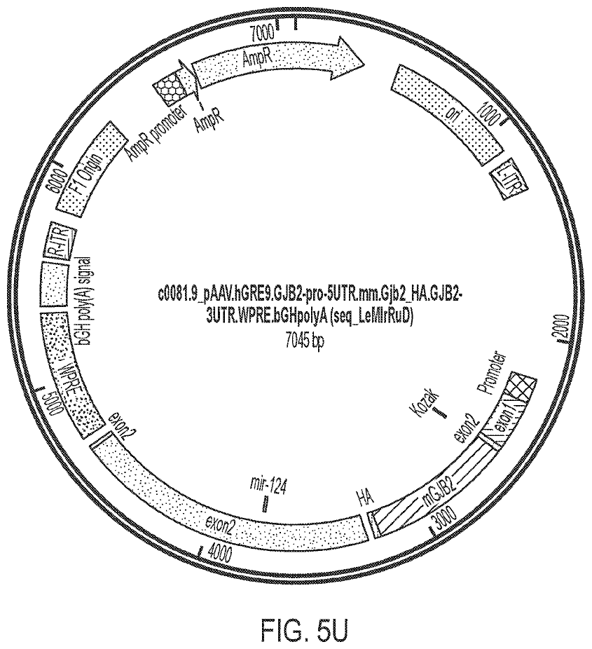

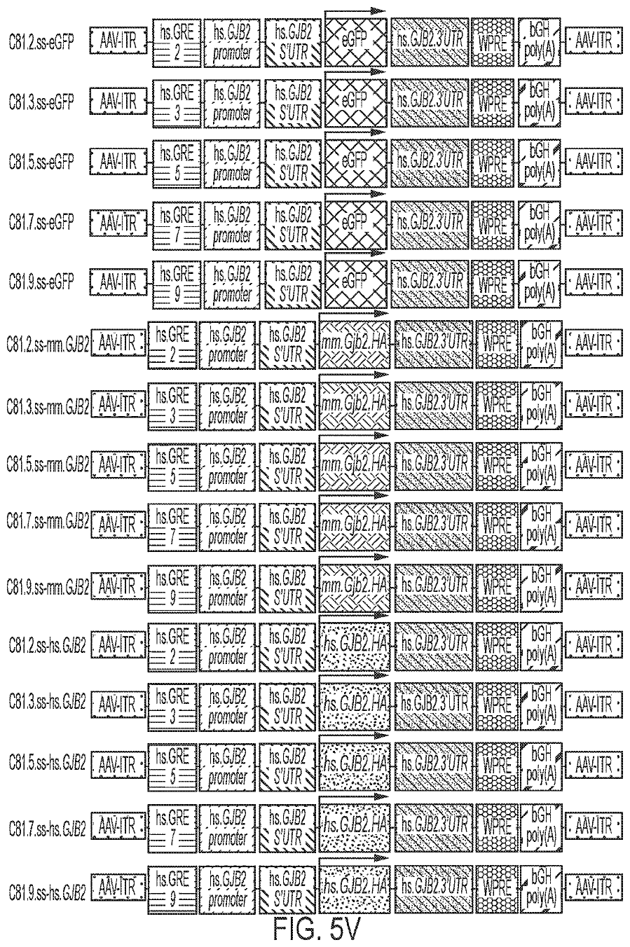

In order to test whether GJB2 GREs listed in Table 3 permit GJB2 expression in cells that normally express it, and prevent GJB2 expression in cells that do not normal express GJB2, the GREs were each incorporated into AAV vectors that drive GFP, human GJB2, or mouse Gjb2 expression under the control of the basal GJB2 promoter, and the GJB2 exon 1 5′ UTR. The vector maps are shown in FIGS. 5A-5U. The vectors include, from 5′ to 3′, an AAV 5′ ITR, a human GJB2 GRE, a GJB2 basal promoter, a human GJB2 exon 1 5′ UTR, a nucleotide sequence encoding an eGFR, a human GJB2 or a mouse Gjb2, and a GJB2 exon 2 3′ UTR. Vector c.81.1 includes human GJB2 GRE1; Vector c.81.2 includes human GJB2 GRE2; Vector c.81.3 includes human GJB2 GRE3; Vector c.81.4 includes human GJB2 GRE4; Vector c.81.5 includes human GJB2 GRE 5; Vector c.81.7 includes human GJB2 GRE7; Vector c.81.8 includes human GJB2 GRE8; Vector c.81.9 includes human GJB2 GRE9 (FIGS. 5A-5U). FIG. 5V shows schematics of c81.2, c81.3, c81.5, c81.7 and c81.9 encoding eGFP, mouse GJB2 and human GJB2 as described above.

The c.81.2, c81.3, c81.5, c81.7, and c81.9 vectors encoding GFP were respectively packaged into rAAVs using AAV9.PHP.B capsid protein and injected through the round window membrane at postnatal day 1 of wild-type mice. The cochlea was fixed for histology at P6, and GFP expression was evaluated in the cochlea tissues.

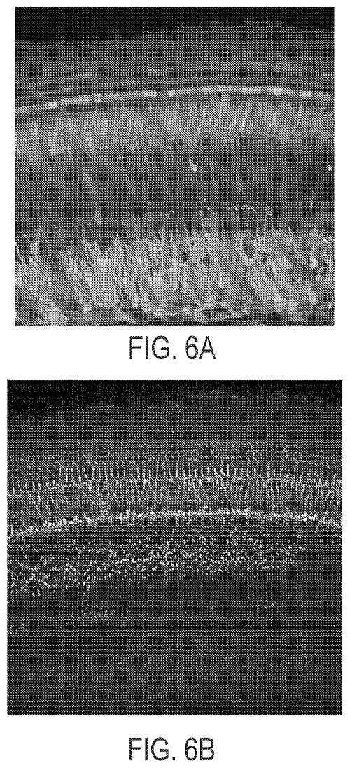

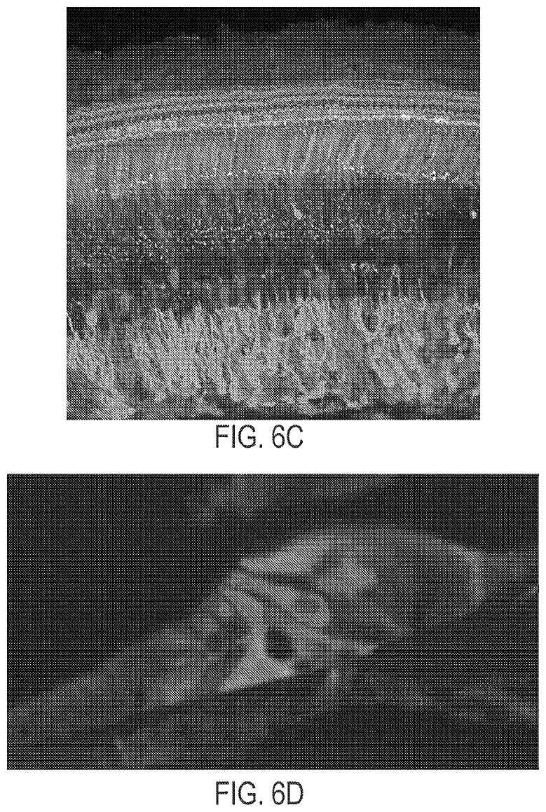

It was found that GJB2 gene regulatory element 5 (GJB2 GRE5, in vector c81.5 encoding eGFP as a reporter) helped target expression of eGFP to GJB2-expressing cells. FIG. 6A shows a fluorescent image of eGFP expressing cells, including a variety of supporting cells in, and medial to, the organ of Corti. FIG. 6B shows antibody label of endogenous GJB2 in the region of the organ of Corti. GJB2 expression largely overlapped that of exogenous eGFP. FIG. 6C is an overlay of FIGS. 6A and 6B, with a third staining of actin, which revealed stereocilia of hair cells. No eGFP was expressed in the hair cells. FIG. 6D shows a frozen section immunofluorescence image of eGFP and a protein marker for hair cells, MYO7A. eGFP was expressed in a variety of supporting cells in the organ of Corti, but did not overlap with MYO7A expression, which was expressed in hair cells. The vectors encoding human GJB2 or mouse GJB2 will be tested for GJB2 expression in the intended cells.

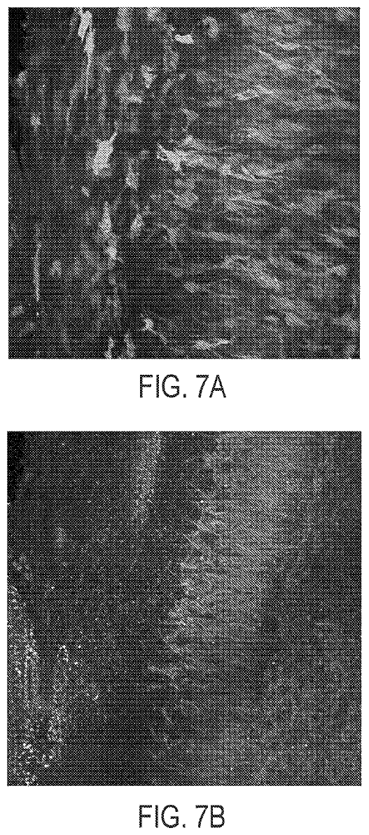

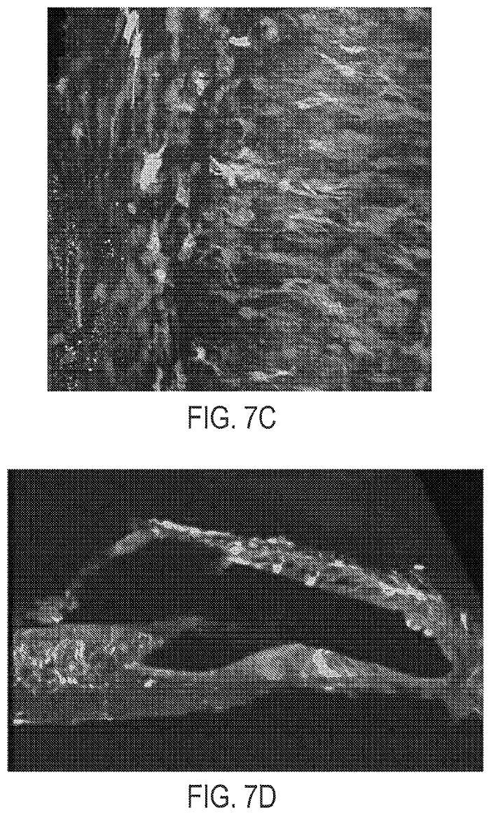

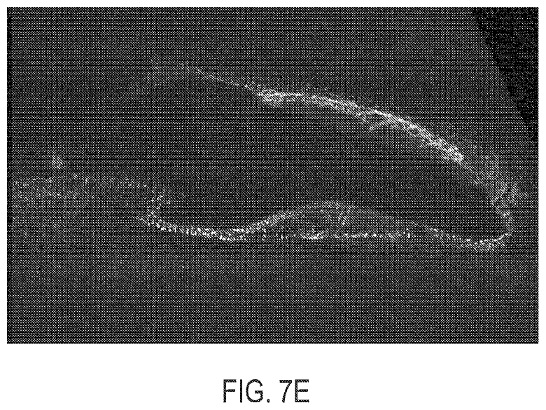

FIGS. 7A-7D show eGFP expression pattern by vector c.81.5 in the lateral wall of the cochlea. FIG. 7A shows eGFP expression in cells including fibrocytes of the lateral wall. FIG. 7B shows an antibody labeling of endogenous GJB2 in the region of the lateral wall. GJB2 expression largely overlaps that of exogenous GFP. FIG. 7C is an overlay image of FIGS. 7A and 7B. Note that eGFP was expressed in the cells expressing Gjb2. FIGS. 7D-7E show frozen section immunofluorescences of GFP (FIG. 7D) and GJB2 in supporting cells of the organ of Corti and fibrocytes of the lateral wall (FIG. 7E).

Human GJB2 enhancers identified based on human deletions are capable of rescue hearing, and similarly does not lead to GJB2 associated toxicity.

### REFERENCES

Buenrostro J D, Giresi P G, Zaba L C, Chang H Y, Greenleaf W J (2013) Transposition of native chromatin for fast and sensitive epigenomic profiling of open chromatin, DNA-binding proteins and nucleosome position. Nature methods 10:1213-1218.
Cohen-Salmon M, Ott T, Michel V, Hardelin J P, Perfettini I, Eybalin M, Wu T, Marcus D C, Wangemann P, Willecke K, Petit C (2002) Targeted ablation of connexin26 in the inner ear epithelial gap junction network causes hearing impairment and cell death. Curr Biol 12:1106-1111.
Crispino G, Di Pasquale G, Scimemi P, Rodriguez L, Galindo Ramirez F, De Siati R D, Santarelli R M, Arslan E, Bortolozzi M, Chiorini J A, Mammano F (2011) BAAV mediated GJB2 gene transfer restores gap junction coupling in cochlear organotypic cultures from deaf Cx26Sox10Cre mice. PloS one 6:e23279.
Deverman B E, Pravdo P L, Simpson B P, Kumar S R, Chan K Y, Banerjee A, Wu W L, Yang B, Huber N, Pasca S P, Gradinaru V (2016) Cre-dependent selection yields AAV variants for widespread gene transfer to the adult brain. Nat Biotechnol 34:204-209.
Feigenspan A, Janssen-Bienhold U, Hormuzdi S, Monyer H, Degen J, Sohl G, Willecke K, Ammermuller J, Weiler R (2004) Expression of connexin36 in cone pedicles and OFF-cone bipolar cells of the mouse retina. J Neurosci 24:3325-3334.
Forge A, Becker D, Casalotti S, Edwards J, Marziano N, Nevill G (2003) Gap junctions in the inner ear: comparison of distribution patterns in different vertebrates and assessment of connexin composition in mammals. J Comp Neurol 467:207-231.
Gyorgy B, Sage C, Indzhykulian A A, Scheffer D I, Brisson A R, Tan S, Wu X, Volak A, Mu D, Tamvakologos P I, Li Y, Fitzpatrick Z, Ericsson M, Breakefield X O, Corey D P, Maguire C A (2017) Rescue of hearing by gene delivery to inner-ear hair cells using exosome-associated AAV. Mol Ther 25:379-391.
Gyorgy B, Meijer E J, Ivanchenko M V, Tenneson K, Emond F, Hanlon K S, Indzhykulian A A, Volak A, Karavitaki K D, Tamvakologos P I, Vezina M, Berezovskii V K, Born R T, O'Brien M, Lafond J F, Arsenijevic Y, Kenna M A, Maguire C A, Corey D P (2018) Gene Transfer with AAV9-PHP.B Rescues Hearing in a Mouse Model of Usher Syndrome 3A and Transduces Hair Cells in a Non-human Primate. Mol Ther Methods Clin Dev 13:1-13.
Iizuka T, Kamiya K, Gotoh S, Sugitani Y, Suzuki M, Noda T, Minowa O, Ikeda K (2015) Perinatal Gjb2 gene transfer rescues hearing in a mouse model of hereditary deafness. Hum Mol Genet 24:3651-3661.
Kelsell D P, Dunlop J, Stevens H P, Lench N J, Liang J N, Parry G, Mueller R F, Leigh I M (1997) Connexin 26 mutations in hereditary non-syndromic sensorineural deafness. Nature 387:80-83.
Kenna M A, Feldman H A, Neault M W, Frangulov A, Wu B L, Fligor B, Rehm H L (2010) Audiologic phenotype and progression in GJB2 (Connexin 26) hearing loss. Arch Otolaryngol Head Neck Surg 136:81-87.
Kikuchi T, Kimura R S, Paul D L, Adams J C (1995) Gap junctions in the rat cochlea: immunohistochemical and ultrastructural analysis. Anat Embryol (Berl) 191:101-118.
Li W, Wu J, Yang J, Sun S, Chai R, Chen Z Y, Li H (2015) Notch inhibition induces mitotically generated hair cells in mammalian cochleae via activating the Wnt pathway. Proceedings of the National Academy of Sciences of the United States of America 112:166-171.
Lin F R, Niparko J K, Ferrucci L (2011) Hearing loss prevalence in the United States. Arch Intern Med 171:1851-1852.
Mason J A, Herrmann K R (1998) Universal infant hearing screening by automated auditory brainstem response measurement. Pediatrics 101:221-228.
Shu Y, Tao Y, Wang Z, Tang Y, Li H, Dai P, Gao G, Chen Z Y (2016) Identification of Adeno-Associated Viral Vectors That Target Neonatal and Adult Mammalian Inner Ear Cell Subtypes. Hum Gene Ther 27:687-699.
Sun Y, Tang W, Chang Q, Wang Y, Kong W, Lin X (2009) Connexin30 null and conditional connexin26 null mice display distinct pattern and time course of cellular degeneration in the cochlea. J Comp Neurol 516:569-579.
Takada Y, Beyer L A, Swiderski D L, O'Neal A L, Prieskorn D M, Shivatzki S, Avraham K B, Raphael Y (2014) Connexin 26 null mice exhibit spiral ganglion degeneration that can be blocked by BDNF gene therapy. Hearing research 309:124-135.
Wang Y, Chang Q, Tang W, Sun Y, Zhou B, Li H, Lin X (2009) Targeted connexin26 ablation arrests postnatal development of the organ of Corti. Biochem Biophys Res Commun 385:33-37.
Watanabe K, Takeda K, Katori Y, Ikeda K, Oshima T, Yasumoto K, Saito H, Takasaka T, Shibahara S (2000) Expression of the Sox10 gene during mouse inner ear development. Brain Res Mol Brain Res 84:141-145.
Wise A K, Tu T, Atkinson P J, Flynn B O, Sgro B E, Hume C, O'Leary S J, Shepherd R K, Richardson R T (2011) The effect of deafness duration on neurotrophin gene therapy for spiral ganglion neuron protection. Hearing research 278:69-76.
Yu Q, Wang Y, Chang Q, Wang J, Gong S, Li H, Lin X (2014) Virally expressed connexin26 restores gap junction function in the cochlea of conditional Gjb2 knockout mice. Gene Ther 21:71-80.
Zelante L, Gasparini P, Estivill X, Melchionda S, D'Agruma L, Govea N, Mila M, Monica M D, Lutfi J, Shohat M, Mansfield E, Delgrosso K, Rappaport E, Surrey S, Fortina P (1997) Connexin26 mutations associated with the most common form of non-syndromic neurosensory autosomal recessive deafness (DFNB1) in Mediterraneans. Hum Mol Genet 6:1605-1609.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. An isolated nucleic acid comprising an expression cassette, wherein the expression cassette comprises a Gap Junction beta 2 (GJB2) gene regulatory element (GRE), and a nucleotide sequence encoding a GJB2 protein.

2. The isolated nucleic acid of claim 1, wherein the GJB2 protein is a human GJB2 protein.

3. The isolated nucleic acid of claim 2, wherein the GJB2 protein comprises an amino acid sequence at least 80% identical to SEQ ID NO: 1.

4. The isolated nucleic acid of any one of claims 1-3, wherein the nucleotide sequence encoding a GJB2 protein comprises a nucleotide sequence at least 80% identical to SEQ ID NO: 2.

5. The isolated nucleic acid of any one of claims 1-4, wherein the expression cassette further comprises a promoter operably linked to the nucleotide sequence encoding a GJB2 protein.

6. The isolated nucleic acid of claim 5, wherein the promoter is a human GJB2 promoter.

7. The isolated nucleic acid of claim 6, wherein the promoter comprises 500 nucleotides of a human GJB2 promoter.

8. The isolated nucleic acid of claim 7, wherein the promoter comprises a nucleic acid sequence at least 80% identical to SEQ ID NO: 5.

9. The isolated nucleic acid of claim 6, wherein the promoter comprises a nucleic acid sequence at least 80% identical to SEQ ID NO: 102, optionally 100% identical to SEQ ID NO: 102.

10. The isolated nucleic acid of any one of claims 1-9, wherein the Gap Junction beta 2 (GJB2) gene regulatory element (GRE) comprises a nucleotide sequence encoding a 5′ UTR.

11. The isolated nucleic acid of claim 9, wherein the 5′ UTR is positioned between the promoter and the nucleotide sequence encoding a GJB2 protein.

12. The isolated nucleic acid of claim 10 or 11, wherein the 5′ UTR comprises about 300 nucleotides of a human GJB2 gene 5′ UTR.

13. The isolated nucleic acid of claim 12, wherein the promoter and the 5′ UTR comprises a nucleotide sequence at least 80% identical to SEQ ID NO: 30.

14. The isolated nucleic acid of any one of claims 1-13, wherein the GJB2 gene regulatory element further comprises an enhancer.

15. The isolated nucleic acid of claim 14, wherein the enhancer is positioned 5′ to the promoter.

16. The isolated nucleic acid of claim 14 or 15, wherein the enhancer is normally present within approximately 200 kb upstream or downstream of a GJB2 gene.

17. The isolated nucleic acid of any one of claims 14-16, wherein the enhancer is normally present within approximately 95 kb of a GJB2 gene.

18. The isolated nucleic acid of any one of claims 14-17, wherein the GJB2 GRE comprises one or more enhancers.

19. The isolated nucleic acid of claim 18, wherein the one or more enhancers are the same enhancers or different enhancers.

20. The isolated nucleic acid of any one of claims 14-19, wherein the enhancer comprises a nucleotide sequence at least 80% identical to a nucleotide sequence or a fragment thereof as set forth in any one of SEQ ID NOs: 6 to 29.

21. The isolated nucleic acid of any one of claims 14-20, wherein the enhancer comprises a nucleotide sequence at least 80% identical to a GJB2 enhancer as set forth in any one of SEQ ID NOs: 37-46.

22. The isolated nucleic acid of claim 21, wherein the enhancer comprises a nucleotide sequence at least 80% identical to SEQ ID NO: 42.

23. An isolated nucleic acid comprising an expression cassette, wherein the expression cassette comprises a Gap Junction beta 2 (GJB2) promoter, and a nucleotide sequence encoding a GJB2 protein.

24. The isolated nucleic acid of claim 23, wherein the GJB2 promoter comprises a nucleic acid sequence at least 80% identical to SEQ ID NO: 102, optionally 100% identical to SEQ ID NO: 102.

25. The isolated nucleic acid of claim 23 or 24, wherein the expression cassette further comprises a 5′ UTR.

26. The isolated nucleic acid of claim 25, wherein the 5′ UTR comprises:
a first nucleic acid sequence at least 80% identical to SEQ ID NO: 103, optionally 100% identical to SEQ ID NO: 103; and/or
a second nucleic acid sequence at least 80% identical to SEQ ID NO: 104, optionally 100% identical to SEQ ID NO: 104.

27. The isolated nucleic acid of any one of claims 23-27, wherein the isolated nucleic acid comprises a nucleic acid sequence at least 80% identical to SEQ ID NO: 105, optionally 100% identical to SEQ ID NO: 105.

28. The isolated nucleic acid of any one of claim 1-27, wherein the isolated nucleic acid is capable of expressing GJB2 in cells that normally express the GJB2 gene.

29. The isolated nucleic acid of claim 28, wherein the isolated nucleic acid is capable of expressing GJB2 in cochlear connective tissue cells and supporting cells of the organ of Corti.

30. The isolated nucleic acid of claim 29, wherein the supporting cell of the organ of Corti are pillar cells, Deiters' cells, Hensen's cells, Claudius cells, inner phalangeal cells, and border cells.

31. The isolated nucleic acid of claim 29, wherein the cochlear connective tissue cells are strial intermediate cells, fibrocytes of the lateral wall and suprastrial zone, basal cells of the stria vascularis, fibrocytes in the spiral ligament, fibrocytes in the spiral limbus, mesenchymal cells lining the bony otic capsule facing the scala vestibuli, and supralimbal dark cells.

32. The isolated nucleic acid of any one of claims 1-31, wherein the expression cassette is flanked by two adeno-associated virus inverted terminal repeats (ITRs).

33. The isolated nucleic acid of claim 32, wherein the AAV ITR is from a serotype selected from the group consisting of AAV1 ITR, AAV2 ITR, AAV3 ITR, AAV4 ITR, AAV5 ITR, and AAV6 ITR.

34. The isolated nucleic acid of claim 32 or 33, wherein the AAV ITR is AAV2 ITR.

35. The isolated nucleic acid of claim 32 or 33, wherein the expression cassette comprises:
a 5′ ITR having a nucleotide sequence at least 80% identical to SEQ ID NO: 106, optionally 100% identical to SEQ ID NO: 106; and/or
a 3′ ITR having a nucleotide sequence at least 80% identical to SEQ ID NO: 107, optionally 100% identical to SEQ ID NO: 107.

36. The isolated nucleic acid of any one of claims 1-35, wherein the expression cassette further comprises a Woodchuck Hepatitis Virus (WHP) Posttranscriptional Regulatory Element (WPRE) 3′ to the nucleotide sequence encoding the GJB2 protein.

37. The isolated nucleic acid of claim 36, wherein the WPRE comprises a nucleotide sequence at least 80% identical to SEQ ID NO: 108, optionally 100% identical to SEQ ID NO: 108.

38. The isolated nucleic acid of any one of claims 1-37, wherein the expression cassette further comprises a nucleotide sequence encoding a 3′ UTR located 3′ of the WPRE.

39. The isolated nucleic acid of claim 38, wherein the 3′ UTR is a GJB2 3′ UTR.

40. The isolated nucleic acid of claim 39, wherein the GJB2 3′ UTR comprises a nucleotide sequence at least 80% identical to SEQ ID NO: 32.

41. The isolated nucleic acid of any one of claims 1-40, wherein the expression cassette further comprises a poly A signal.

42. The isolated nucleic acid of claim 41, wherein the poly A signal is a bovine growth hormone poly A signal.

43. The isolated nucleic acid of claim 41, wherein the poly A signal comprises a nucleotide sequence at least 80% identical to SEQ ID NO: 109, optionally 100% identical to SEQ ID NO: 109.

44. An isolated nucleic acid comprising a nucleotide sequence at least 80% identical to SEQ ID NO: 110 or 111, optionally 100% identical to SEQ ID NO: 110 or 111.

45. A vector comprising the isolated nucleic acid of any one of claims 1-44.

46. The vector of claim 45, wherein the vector is a plasmid or a viral vector.

47. The vector of claim 46, wherein the viral vector is an AAV vector.

48. A vector comprising from 5′ to 3′:
(a) a 5′ ITR;
(b) a GJB2 promoter, or a basal GJB2 promoter sequence thereof;
(c) a GJB2 5′ UTR;
(d) a nucleotide sequence encoding a GJB2 protein;
(e) a GJB2 3′ UTR;
(f) a bovine growth hormone poly A signal; and
(g) a 3′ ITR.

49. A vector comprising from 5′ to 3′:
(a) a 5′ ITR;
(b) a GJB2 enhancer;
(c) a GJB2 promoter, or a basal GJB2 promoter sequence thereof;
(d) a GJB2 5′ UTR;
(e) a nucleotide sequence encoding a GJB2 protein;
(f) a GJB2 3′ UTR;
(g) a bovine growth hormone poly A signal; and
(h) a 3′ ITR.

50. A recombinant adeno-associated virus (rAAV) comprising:
(i) a capsid protein; and
(ii) the isolated nucleic acid of any one of claims 1-44.

51. A recombinant adeno-associated virus (rAAV) comprising:
(i) a capsid protein; and
(ii) an isolated nucleic acid comprising:
(a) a 5′ ITR;
(b) a GJB2 promoter, or a basal GJB2 promoter sequence thereof;
(c) a GJB2 5′ UTR;
(d) a nucleotide sequence encoding a GJB2 protein;
(e) a GJB2 3′ UTR;
(f) a bovine growth hormone poly A signal; and
(g) a 3′ ITR.

52. A recombinant adeno-associated virus (rAAV) comprising:
(i) a capsid protein; and
(ii) an isolated nucleic acid comprising:
(a) a 5′ ITR;
(b) a GJB2 enhancer;
(c) a GJB2 promoter, or a basal GJB2 promoter sequence thereof;
(d) a GJB2 5′ UTR;
(e) a nucleotide sequence encoding a GJB2 protein;
(f) a GJB2 3′ UTR;
(g) a bovine growth hormone poly A signal; and
(h) a 3′ ITR.

53. The rAAV of any one of claims claim 50-52, wherein the rAAV has tropism for a subset of cochlear cells that normally express the GJB2 gene.

54. The rAAV of any one of claims 50-53, wherein the rAAV has tropism for cells of the inner ear.

55. The rAAV of any one of claims 50-54, wherein the capsid protein is an AAV1 capsid protein, an AAV2 capsid protein, an AAV5 capsid protein, an AAV7 capsid protein, an AAV8 capsid protein, an AAV9 capsid protein, an AAV-S capsid protein, or a variant thereof.

56. The rAAV of any one of claims 50-55, wherein the AAV capsid is AAV9.PHP.B, AAV9.PHP.eB, or AAV-S.

57. The rAAV of claim 56, wherein the AAV capsid protein is AAV-S.

58. A cell comprising the isolated nucleic acid of any one of claims 1-44, the vector of any one of claims 45-49, or the rAAV of any one of claims 50-57.

59. A pharmaceutical composition comprising the isolated nucleic acid of any one of claims 1-44, the vector of any one of claims 45-49, the rAAV of any one of claims 50-57, or the cell of claim 58.

60. The pharmaceutical composition of claim 59 further comprising a pharmaceutically acceptable carrier.

61. A method for specifically expressing GJB2 in cells that normally expresses the GJB2 gene in a subject, the method comprising administering to the subject an effective amount of the isolated nucleic acid of any one of claims 1-44, the vector of any one of claims 45-49, the rAAV of any one of claims 50-57, the cell of claim 58, or the pharmaceutical composition of claim 59 or 60.

62. A method for treating Non-syndromic Hearing Loss and Deafness (DFNB1) in a subject in need thereof, the method comprising administering to the subject an effective amount of the isolated nucleic acid of any one of claims 1-44, the vector of any one of claims 45-49, the rAAV of any one of claims 50-57, the cell of claim 58, or the pharmaceutical composition of claim 59 or 60.

63. A method for treating a GJB2-associated disease in a subject in need thereof, the method comprising administering to the subject an effective amount of the isolated nucleic acid of any one of claims 1-44, the vector of any one of claims 45-49, the rAAV of any one of claims 50-57, the cell of claim 58, or the pharmaceutical composition of claim 59 or 60.

64. The method of any one of claims 61-63, wherein the subject is a mammal.

65. The method of claim 64, wherein the mammal is a human.

66. The method of claim 64, wherein the mammal is a non-human mammal.

67. The method of claim 66, wherein the non-human mammal is mouse, rat, or non-human primate.

68. The method of any one of claims 61-67, wherein the hearing loss is associated with a mutation in the GJB2 gene.

69. The method of claim 68, wherein the mutation in the GJB2 gene is a point mutation, a missense mutation, a nonsense mutation, a deletion, an insertion, or a combination thereof.

70. The method of claim 69, wherein the subject is human; and the mutation is a mutation listed in Table 2 or a combination thereof.

71. The method of claim 69 or 70, wherein the mutation is c 101.T>C or Del35G.

72. The method of any one of claims 61-71, wherein the step of administrating results in expression of GJB2 protein in cochlear connective tissue cells and supporting cells of the organ of Corti.

73. The method of claim 72, wherein the supporting cells of the organ of Corti are pillar cells, Deiters' cells, Hensen's cells, Claudius cells, inner phalangeal cells, and border cells.

74. The method of claim 72, wherein the cochlear connective tissue cells are strial intermediate cells, fibrocytes of the lateral wall and suprastrial zone, basal cells of the stria vascularis, fibrocytes in the spiral ligament, fibrocytes in the spiral limbus, mesenchymal cells lining the bony otic capsule facing the scala vestibuli, and supralimbal dark cells.

75. The method of any one of claims 61-74, wherein the administration is via injection.

76. The method of claim 75, wherein the injection is through the round window membrane of the cochlea, into the scala media of the cochlea, into the scala vestibuli of the cochlea, into a semicircular canal of the inner ear, or into the saccule or the utricle of the inner ear.
